(19) United States
(12) Patent Application Publication
(10) Pub. No.: US 2024/0315192 A1
(43) Pub. Date: Sep. 26, 2024

(54) CELLULOSE-SYNTHASE-LIKE ENZYMES AND USES THEREOF

(71) Applicant: YEDA RESEARCH AND DEVELOPMENT CO., LTD., Rehovot (IL)

(72) Inventors: Asaph AHARONI, Tel-Aviv (IL); Prashant SONAWANE, Rehovot (IL); Maxim ITKIN, Rehovot (IL); Adam JOZWIAK, Rehovot (IL)

(73) Assignee: YEDA RESEARCH AND DEVELOPMENT CO., LTD., Rehovot (IL)

(21) Appl. No.: 18/735,258

(22) Filed: Jun. 6, 2024

Related U.S. Application Data
(63) Continuation-in-part of application No. 17/272,685, filed on Mar. 2, 2021, now Pat. No. 12,041,907.
Application No. 17/272,685 is a continuation of application No. 16/123,248, filed on Sep. 6, 2018, now Pat. No. 10,806,119.

(30) Foreign Application Priority Data
Jul. 25, 2019 (IL) 268269

(51) Int. Cl. A01H 6/82; C12N 9/10; C12N 15/82

(52) U.S. Cl. CPC A01H 6/82; C12N 9/1059; C12N 15/8218; C12N 15/8243; C12N 15/8247

(57) ABSTRACT

Provided herein are genetically modified cells and genetically modified plants having increased or decreased expression of a cellulose synthase like G (CSLG) enzyme. These cells and plants may have an increased or decreased content a steroidal alkaloid, a steroidal saponin, or a triterpenoid saponin, compared to a corresponding unmodified cell or plant. Also provided herein are methods of producing a steroidal alkaloid, a steroidal saponin, or a triterpenoid saponin in a genetically modified cell, as well as methods of reducing the content of a steroidal alkaloid, a steroidal saponin, or a triterpenoid saponin in a cell of a plant or a plant part, and methods of increasing the content of a steroidal alkaloid, a steroidal saponin, or a triterpenoid saponin in a cell of a plant or a plant part.

### CROSS REFERENCE TO RELATED APPLICATIONS

This application is a Continuation in Part application of U.S. application Ser. No. 17/272,685 filed Mar. 2, 2021, which filed as a National Phase application of PCT International Application No. PCT/IL2019/051000, International Filing Date Sep. 5, 2019, which claims the benefit of U.S. patent application Ser. No. 16/123,248, filed Sep. 6, 2018 and issued as U.S. Pat. No. 10,806,119 on Oct. 20, 2020 and claims the benefit of Israel Patent Application Serial Number 268269, filed Jul. 25, 2019, which are incorporated in their entirety herein by reference.

### SEQUENCE LISTING STATEMENT

The instant application contains a Sequence Listing which has been submitted electronically in ST.26.XML format and is hereby incorporated by reference in its entirety. The .XML formatted sequence listing was created on May 30, 2024, is named P-603031-US1_SL.xml and is 256,487 bytes in size.

### FIELD OF THE INVENTION

The disclosure relates in general to the field of altering the content of at least one steroidal alkaloid; at least one steroidal saponin; or at least one triterpenoid saponin in at least one cell of a plant or a plant part. In one embodiment, the present disclosure describes the cells or plants comprising altered content of at least one steroidal alkaloid; at least one steroidal saponin; or at least one triterpenoid saponin, and methods of producing same.

### BACKGROUND

The plant kingdom produces hundreds of thousands of different small compounds, often genus or family specific. The massive structural diversity of more than 300,000 plant specialized metabolites (SMs) is generated through assorted modifications of their core structure. Plant SMs are most frequently glycosylated, and this glycosylation significantly impacts their compartmentalization, activity, solubility, stability, and toxicity. They are low molecular weight, nitrogen-containing, organic compounds, typically with a heterocyclic structure.

The broad group of saponin-alkaloid compounds is widespread in plants and derived from the cytosolic mevalonic acid isoprenoid biosynthetic pathway. Saponins and steroidal alkaloids (SAs) are two large classes of SMs produced by plants.

Saponins are a large group of SMs found in countless plant species and more than 100 families, representing a lipophilic triterpenoid or steroidal backbone (aglycone) decorated with one or more glycoside moieties.

As suggested by their name (Latin sāpō means soap), saponins display soap like properties in aqueous solutions due to their amphipathic structure. The unique physicochemical properties of these compounds provide a broad spectrum of functions in plants including antifungal, antibacterial and insecticidal. The activity of saponins depends on the structure of the aglycone but in many cases even more on the attached sugars. For example, oleanolic and medicagenic acid (MA) derived saponins display hemolytic activity due to the presence of a carboxyl at position C-28 while Avenacin A-1 requires L-arabinose attached at the C-3 position to be fungicidal. Moreover, cholesterol-derived steroidal saponins are widespread in the plant kingdom, are highly diverse in structures, and can be either saturated (e.g., sarasapogenin) or unsaturated (e.g., diosgenin) in the C-5,6 position.

Plentiful reports underline saponins' benefits to human health and medical applications starting from anti-inflammatory and immune-boosting (adjuvant) to anti-cancer properties. UDP-glycosyltransferases (UGTs) members of the carbohydrate-active enzyme (CAZY) glycosyltransferase 1 (GT1) superfamily carry out sugar transfer reactions on saponins and all other SMs classes. The most common saccharides decorating saponins include D-glucose, D-galactose, L-arabinose, D-glucuronic acid, D-xylose, L-rhamnose and D-fucose.

The presence of glucuronic acid attached at position C-3 of the sapogenin is particularly common for species of the Caryophyllales order, yet, the enzyme involved remains unknown.

Thus, there remains an unmet need for knowledge regarding the enzymes involved in the metabolic pathway to produce saponins, including triterpenoid saponins, including knowledge of Cellulose Synthase Like Gs that attach glucuronic acid to, for example, quillaic acid. Once known, key enzymes could be regulated in a plant in order to alter the content of naturally produced products. For example, an enzyme could be over-expressed or have increased stability, thereby producing a natural sweetener at increased quantities, or an enzyme could be down regulated at the gene level to reduce production of compounds adding a bitter taste to plant products, for example quinoa. Further, there remains an unmet need for methods to produce these saponins in heterologous systems in order to provide commercial quantities of high value saponins, for example but not limited to triterpenoid saponins. These high value saponins may be used for example as, but not limited to, sweeteners, foaming agents, emulsifiers, preservatives, anti-carcinogens, hypocholesterolemic agents, anti-inflammatory agents, anti-oxidants, biological adjuvants, anti-microbial agents, insecticidal agents, antifeedants, or anti-fungal agents, or any combination thereof.

In addition, some SMs, such as alkaloids, are often referred to as secondary metabolites, because they are not vital to cells that produce them, but contribute to the overall fitness of the organism. Alkaloids are low molecular weight nitrogen-containing organic compounds, typically with a heterocyclic structure, and their biosynthesis in plants is tightly controlled during development and in response to stress and pathogens.

For example, steroidal alkaloids (SAs), occasionally known as “Solanum alkaloids” due to their high prevalence in numerous members of the Solanales order, have been found to be common to numerous plants in a wide range of families. SAs have diverse structural composition and biological activity. They are low molecular weight, nitrogen-containing, cholesterol-derived organic compounds, typically with a heterocyclic structure consisting of a C-27 cholestane skeleton and a heterocyclic nitrogen component.

Estimated in the order of 1350 species, Solanum is one of the largest genera of flowering plants, representing about half of the species in the Solanaceae family (which is in the Solanales order). Solanum species include food plants, such as tomato (Solanum lycopersicum), potato (Solanum tuberosum), bittersweet (Solanum dulcamara), and eggplant (Solanum melogena), and the Solanaceae also includes the Capsicum genus (e.g., peppers), as well as many other genera. Steroidal alkaloids are also produced by a large number of species in the Liliaceae family. As a result, SAs have been the subject of extensive investigations (Eich E. 2008. Solanaceae and Convolvulaceae—secondary metabolites: biosynthesis, chemotaxonomy, biological and economic significance: a handbook. Berlin: Springer).

Consisting of a C-27 cholestane skeleton and a heterocyclic nitrogen component, SAs were suggested to be synthesized in the cytosol from cholesterol. Conversion of cholesterol to the alkamine SA should require several hydroxylation, oxidation, and transamination reactions (Eich 2008, supra), and in most cases, further glycosylation to form steroidal glycoalkaloids (SGAs) (Arnqvist L. et al. 2003. Plant Physiol. 131:1792-1799). Glycosylation of SAs produces steroidal glycoalkaloids (SGAs), in which the added oligosaccharide moiety components directly conjugate to the hydroxyl group at C-3-beta (C-3β) of the alkamine steroidal skeleton (aglycone). SGA biosynthesis depends on genes encoding UDP-glycosyltransferases (UGTs) that decorate the aglycone with various oligosaccharide moieties, including D-glucose, D-galactose, L-rhamnose, D-xylose, and L-arabinose, the first two monosaccharides being the predominant units.

SGAs are produced by numerous members of the Solanaceae family, as well as many other families of plants. Examples of these compounds include alpha-tomatine and dehydrotomatine in tomato (Solanum lycopersicum), alpha-chaconine and alpha-solanine in potato (Solanum tuberosum), and alpha-solamargine and alpha-solasonine in eggplant (Solanum melongena). SGAs are also found in various types of pepper in the genus Capsicum. More than 100 SGAs have been identified in tomatoes (Itkin et al., 2011, Plant Cell 23:4507-4525), and more than 50 have been identified in potatoes (Shakya and Navarre, 2008, J. Agric. Food Chem. 56:6949-6958). Eggplant also contains at least one variety of SGA (Friedmann, 2006, J. Agric. Food Chem. 54:8655-8681).

SAs and SGAs play a role in protecting plants against a broad range of pathogens and are known as phytoanticipins (antimicrobial compounds). SGAs contribute to plant resistance to a wide range of pathogens and predators, including bacteria, fungi, oomycetes, viruses, insects, and larger animals. Some SGAs in edible parts of plants are considered to be anti-nutritional compounds to humans and other mammals due to their toxic effects. For example, the SGAs alpha-chaconine and alpha-solanine are the principle toxic substances in potato, may cause gastrointestinal and neurological disorders and, at high concentrations, may be lethal to humans. Mechanisms of toxicity include disruption of membranes an dinhibition of acetylcholine esterase activity (Roddick J. G. 1989. Phytochemistry 28:2631-2634). For this reason, total SGA levels exceeding 200 mg/kg fresh weight of edible tuber are deemed unsafe for human consumption.

There is an ongoing attempt to elucidate the biosynthesis pathway of steroidal alkaloids and to control their production. U.S. Pat. No. 5,959,180 discloses DNA sequences from potato which encode the enzyme solanidine UDP-glucose glucosyltransferase (SGT). Further disclosed are means and methods for inhibiting the production of SGT and thereby reduce glycoalkaloid levels in Solanaceous plants, for example potato.

Similarly, U.S. Pat. Nos. 7,375,259 and 7,439,419 disclose nucleic acid sequences from potato that encode the enzymes UDP-glucose:solanidine glucosyltransferase (SGT2) and β-solanine/β-chaconine rhamnosyltransferase (SGT3), respectively. Recombinant DNA molecules containing the sequences, and use thereof, in particular, use of the sequences and antisense constructs to inhibit the production of SGT2/SGT3 and thereby reduce levels of the predominant steroidal glycoalkaloids α.-chaconine and α-solanine in Solanaceous plants such as potato are also described.

Recently, three glycosyltransferases were identified that are putatively involved in the metabolism of tomato steroidal alkaloids (GLYCOALKALOID METABOLISM 1-3 (GAME1-3). More specifically, alterations in GAME1 expression modified the SA profile in tomato plants in both reproductive and vegetative parts. It is suggested that these genes are involved in the metabolism of tomatidine (the α-tomatine precursor) partially by generating the lycotetraose moiety (Itkin et al., 2011, supra).

International Patent Application Publication No. WO 00/66716 discloses a method for producing transgenic organisms or cells comprising DNA sequences which code for sterol glycosyltransferases. The transgenic organisms include bacteria, fungi, plants and animals, which exhibit an increased production of steroid glycoside, steroid alkaloid and/or sterol glycoside compared to that of wild-type organisms or cells. The synthesized compounds are useful in the pharmaceutical and foodstuff industries as well as for protecting plants.

U.S. Patent Application Publication No. 2012/0159676 discloses a gene encoding a glycoalkaloid biosynthesis enzyme derived from a plant belonging to the family Solanaceae for example potato (Solanum tuberosum). A method for producing/detecting a novel organism using a gene encoding the protein is also disclosed.

U.S. Patent Application Publication No. 2013/0167271 and International Application Publication No. WO 2012/095843 relate to a key gene in the biosynthesis of steroidal saponins and steroidal alkaloids and to means and methods for altering the gene expression and the production of steroidal saponins and steroidal alkaloids.

A paper of inventors of the present invention, published after the priority date of the present invention, describes an array of 10 genes that partake in SGA biosynthesis. 5-7 of the genes were found to exist as a cluster on chromosome 7 while additional two reside adjacent in a duplicated genomic region on chromosome twelve. Following systematic functional analysis, a novel SGA biosynthetic pathway starting from cholesterol up to the tetrasaccharide moiety linked to the tomato SGA aglycone has been proposed (Itkin M. et al., 2013 Science 341(6142):175-179).

It has also been found that the plant cholesterol biosynthetic pathway overlaps with phytosterol metabolism (Sonawane et al. 2016. Nat. Plants 3: 16205). For example, cholesterol ((3-beta)-cholest-5-en-3-ol) is a sterol (or modified steroid), a type of lipid molecule biosynthesized by all animal cells as an essential structural component of all animal cell membranes and essential to membrane structural integrity and fluidity, enabling animal cells to function without a cell wall. It is also a precursor for the biosynthesis of steroid hormones, bile acid, and vitamin D, as well as saponins, SAs, and SGAs. Cholestanol (5-beta-cholestan-3-beta-ol; coprostanol; 5-beta-coprostanol) is a cholesterol derivative found in feces and other biological matter. Cholestanol is a 27-carbon stanol formed from the biohydrogenation of cholesterol in the gut of most higher animals (birds and mammals) by the conversion of cholesterol.

Plants make cholesterol in very small amounts, but also manufacture phytosterols (which include plant sterols and stanols, similar to cholesterol and cholestanol), which can compete with cholesterol for reabsorption in the intestinal tract, thus potentially reducing cholesterol reabsorption. Cholesterol is often used in the manufacture of medicines, cosmetics, and other applications. There is an increased interest in producing higher levels of both plant phytosterols and plant-based cholesterol.

For example, in tomato (e.g., Solanum lycopersicum, Solanum pennelii), α-tomatine and dehydrotomatine represent the major SGAs accumulating predominantly in green tissues; young and mature leaves, flower buds, skin and seeds of immature and mature green fruit. Dehydrotomatidine (i.e. tomatidenol) is the first SA aglycone formed in SGA biosynthesis which could further be hydrogenated at the C-5 position to form tomatidine. Both aglycones are further glycosylated (tetrasaccharide moiety, i.e., lycotetrose) to produce dehydrotomatine and α-tomatine, respectively. Thus, the SGA pathway branches at dehydrotomatidine for either formation of tomatidine derived SGAs or glycosylated dehydrotomatine derivatives. Notably, dehydrotomatidine and tomatidine are only different in their structures by the presence or absence of the double bond at the C-5 position. The conversion of dehydrotomatidine to tomatidine was hypothesized in the past as a single reaction catalyzed by a hypothetical hydrogenase. In most tomato plant tissues, the relative portion of dehydrotomatine as compared to α-tomatine ranges from ˜2.5-˜10%. As tomato fruit matures and reaches to the red stage, the entire pool of α-tomatine and dehydrotomatine is largely being converted to esculeosides (major SGAs) and dehydroesculeosides (minor SGAs), respectively.

In cultivated potato (e.g., Solanum tuberosum), α-chaconine and α-solanine are the major SGAs sharing the same aglycone, solanidine (in which a C-5,6 double bond is present) and possess chacotriose and solatriose moieties, respectively. As there is no demissidine or demissine detected in cultivated potatoes, it was suggested that a hydrogenase enzyme able to convert solanidine to demissidine is lacking in these species. Several wild potato species (e.g. S. demissum, S. chacoense, S. commersonii) and their somatic hybrids (S. brevidens X S. tuberosum), predicted to contain an active hydrogenase, do produce demissidine or its glycosylated form, demissine being one of their major SGAs.

In eggplant (e.g., Solanum melongena), α-solamargine and α-solasonine are the most abundant SGAs derived from the solasodine aglycone (in which a C-5,6 double bond is present); while some wild Solanum species, e.g. S. dulcamara (bittersweet) produce soladulcidine or its glycosylated forms, soladulcine A and β-soladulcine (C-5,6 double bond is absent), as major SGAs from the solasodine aglycone.

In addition to SGAs, many Solanum species (e.g., eggplant) also produce cholesterol-derived unsaturated or saturated steroidal saponins. Unsaturated and saturated steroidal saponins are widespread in the plant kingdom, especially among monocots, e.g. the Agavaceae (e.g., agave and yucca), Asparagaceae (e.g., asparagus), Dioscoreaceae and Liliaceae families. Similar to SGAs, steroidal saponins are highly diverse in structures and could be either saturated (e.g. sarasapogenin) or unsaturated (e.g. diosgenin) in the C-5,6 position.

Cholesterol, the main sterol produced by all animals, serves as a key building block in the biosynthesis of SGAs. An array of tomato and potato GLYCOALKALOIDMETABOLISM (GAME) genes participating in core SGA biosynthesis starting from cholesterol were reported in recent years. The tomato SGAs biosynthetic pathway can be divided into two main parts. In the first, the SA aglycone is formed from cholesterol by the likely action of the GAME6, GAME8, GAME11, GAME4 and GAME12 enzymes. The second part results in the generation of SGA through the action of UDP-glycosyltransferases (UGTs): GAME1, GAME2, GAME17 and GAME18 in tomato, and STEROL ALKALOID GLYCOSYL TRANSFERASE1 (SGT1), SGT2 and SGT3 in potato.

Thus, there remains an unmet need for knowledge regarding the enzymes involved in the metabolic pathway to produce saponins, including triterpenoid saponins, including knowledge of Cellulose Synthase Like Gs that attach glucuronic acid to, for example, quillaic acid. Once known, key enzymes could be regulated in a plant in order to alter the content of naturally produced products. For example, an enzyme could be over-expressed or have increased stability, thereby producing a natural sweetener at increased quantities, or an enzyme could be down regulated at the gene level to reduce production of compounds adding a bitter taste to plant products, for example quinoa. Further, there remains an unmet need for methods to produce these saponins in heterologous systems in order to provide commercial quantities of high value saponins, for example but not limited to triterpenoid saponins. These high value saponins may be used for example as, but not limited to, sweeteners, foaming agents, emulsifiers, preservatives, anti-carcinogens, hypocholesterolemic agents, anti-inflammatory agents, anti-oxidants, biological adjuvants, anti-microbial agents, insecticidal agents, antifeedants, or anti-fungal agents, or any combination thereof.

In addition, the demand for higher food quantities and food with improved quality continues to increase. Improved nutritional qualities as well as removal of antinutritional traits are both of high demand. In the course of crop domestication, levels of anti-nutrients were reduced by breeding, However, many crop plants still contain significant amount of antinutritional substances, particularly steroidal glycoalkaloids.

Alternatively, the ability to manipulate the synthesis of these SGAs would provide the means to develop, through classical breeding or genetic engineering, crops with modified levels and composition of SGAs, conferring on the plant an endogenous chemical barrier against a broad range of insects and other pathogens.

In addition, there is a demand both for plant-based cholesterols and, conversely, for plants with increased levels of phytocholesterols or other phytosterols.

Thus, there is also a demand for, and it would be highly advantageous to have, means and methods for controlling the production of saponins, steroidal alkaloids, and steroidal glycoalkaloids with beneficial, particularly therapeutic, effects.

The disclosure provided herein below, meets these unmet needs by describing in some embodiments, at least the knowledge that plant SMs, more specifically saponins, are not merely glycosylated by UGT 1 family enzymes. It appears that proteins related to Cellulose Synthases (CESA), renowned for their role in primary cell wall biosynthesis, enable the attachment of glucuronic acid at the C-3 position of saponins. Metabolic evolution through neofunctionalization to a Cellulose Synthase Like (CSL) enzyme possessing a new form of triterpenoid glycosyl transferase activity was accompanied by altering its subcellular localization, directing it to the endoplasmic reticulum (ER). Modulation of this protein also resulted in strict control of the production of steroidal saponins and steroidal glycoalkaloids, while gene silencing resulted in accumulation of cholesterol pools.

The disclosure provided herein below, also meets these unmet needs by describing in some embodiments, at least the knowledge of key genes and enzymes in the biosynthesis pathway converting cholesterol to steroidal saponins, triterpenoid saponins, steroidal alkaloids, and steroidal glycoalkaloids.

### SUMMARY

According to one aspect, provided herein is a genetically modified cell having increased expression of at least one heterologous gene compared to a corresponding unmodified cell, said at least one heterologous gene encoding a cellulose synthase like G (CSLG) enzyme, wherein said genetically modified cell comprises an increased content of at least one steroidal alkaloid, a derivative thereof, a metabolite thereof, or a biosynthetic intermediate thereof, of at least one steroidal saponin, a derivative thereof, a metabolite thereof, or a biosynthetic intermediate thereof, or of at least one triterpenoid saponin, a derivative thereof, a metabolite thereof, or a biosynthetic intermediate thereof, compared to a corresponding unmodified cell.

According to another aspect, provided herein is a genetically modified plant comprising at least one cell having altered expression of at least a cellulose synthase like G (CSLG) gene compared to the expression of CSLG in a corresponding unmodified plant, and wherein the genetically modified plant has an altered content of at least one steroidal alkaloid, a derivative thereof a metabolite thereof, of at least one steroidal saponin, a derivative thereof, a metabolite thereof, or a biosynthetic intermediate thereof, or of at least one triterpenoid saponin, a derivative thereof, a metabolite thereof, or a biosynthetic intermediate thereof compared to the corresponding unmodified plant.

According to another aspect, provided herein is a genetically modified plant comprising at least one cell having decreased expression of at least a cellulose synthase like G (CSLG) gene compared to the expression of CSLG in a corresponding unmodified plant, and wherein the genetically modified plant has a decreased content of at least one steroidal alkaloid, a derivative thereof a metabolite thereof, of at least one steroidal saponin, a derivative thereof, a metabolite thereof, or a biosynthetic intermediate thereof, or of at least one triterpenoid saponin, a derivative thereof, a metabolite thereof, or a biosynthetic intermediate thereof as compared to the corresponding unmodified plant.

According to still another aspect, provided herein is a method of producing a steroidal alkaloid, a steroidal saponin, or a triterpenoid saponin in a genetically modified cell, the method comprising: (a) introducing an at least one heterologous gene into said cell, said at least one heterologous gene encoding a cellulose synthase like G (CSLG) enzyme, wherein said heterologous gene is optionally comprised in a vector; and (b) expressing said at least one heterologous gene in said cell; wherein said cell comprises an increased content of at least one steroidal alkaloid, at least one steroidal saponin, or at least one triterpenoid saponin compared to a corresponding unmodified cell.

According to yet another aspect, provided herein is a method of reducing the content of at least one steroidal alkaloid, a derivative thereof, a metabolite thereof, or a biosynthetic intermediate thereof, of at least one steroidal saponin, a derivative thereof, a metabolite thereof, or a biosynthetic intermediate thereof, or of at least one triterpenoid saponin, a derivative thereof, a metabolite thereof, or a biosynthetic intermediate thereof, in at least one cell of a plant or a plant part, the method comprising genetically modifying said at least one plant cell, said genetic modification comprising: (a) transforming said at least one plant cell with at least one silencing molecule targeted to a nucleic acid gene sequence encoding a Cellulose Synthase Like G (CSLG) enzyme; or (b) mutagenizing at least one nucleic acid sequence encoding a Cellulose Synthase Like G (CSLG) enzyme, wherein the mutagenesis comprises introducing (1) one or more point mutations into the nucleic acid sequence, (2) deletions within the nucleic acid sequence, or (3) insertions within the nucleic acid, or (4) any combination thereof, wherein said introducing comprising mutagenizing coding or non-coding sequence; wherein expression of the gene encoding the CSLG enzyme is reduced in the genetically modified plant cell compared to its expression in a corresponding unmodified plant cell, wherein the plant comprising said genetically modified cell comprises reduced content of at least one steroidal alkaloid, a derivative thereof, a metabolite thereof, or a biosynthetic intermediate thereof, of at least one steroidal saponin, a derivative thereof, a metabolite thereof, or a biosynthetic intermediate thereof, or of at least one triterpenoid saponin, a derivative thereof, a metabolite thereof, or a biosynthetic intermediate thereof, compared to the corresponding unmodified plant.

According to yet another aspect, provided herein is a method of increasing the content of at least one steroidal alkaloid, a derivative thereof, a metabolite thereof, or a biosynthetic intermediate thereof, of at least one steroidal saponin, a derivative thereof, a metabolite thereof, or a biosynthetic intermediate thereof, or of at least one triterpenoid saponin, a derivative thereof, a metabolite thereof, or a biosynthetic intermediate thereof, in at least one cell of a plant or plant part, the method comprising genetically modifying said at least one plant cell, said genetic modification comprising: (a) mutagenizing at least one nucleic acid sequence encoding a Cellulose Synthase Like G (CSLG) enzyme, wherein the mutagenesis comprises introducing (1) one or more point mutations into the nucleic acid sequence, (2) deletions within the nucleic acid sequence, or (3) insertions within the nucleic acid, or (4) any combination thereof, wherein said introducing comprising mutagenizing coding or non-coding sequence; and (b) expressing said nucleic acid encoding said CSLG; wherein the plant comprising said genetically modified cell comprises increased content of at least one steroidal alkaloid, a derivative thereof, a metabolite thereof, or a biosynthetic intermediate thereof, of at least one steroidal saponin, a derivative thereof, a metabolite thereof, or a biosynthetic intermediate thereof, or of at least one triterpenoid saponin, a derivative thereof, a metabolite thereof, or a biosynthetic intermediate thereof, compared to the corresponding unmodified plant.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The subject matter related to methods of production of steroidal glycoalkaloids, steroidal saponins, and triterpenoid saponins and uses thereof is particularly pointed out and distinctly claimed in the concluding portion of the specification. The methods of production of these steroidal glycoalkaloids, steroidal saponins, and triterpenoid saponins and uses thereof, however, both as to organization and method of operation, together with objects, features, and advantages thereof, may best be understood by reference to the following detailed description when read with the accompanying drawings in which:

FIG. 1 shows the proposed biosynthetic pathway of steroidal glycoalkaloids in the triterpenoid biosynthetic pathway in Solanaceous plant from cholesterol toward α-tomatine. Dashed and solid arrows represent multiple or single enzymatic reactions in the pathway, respectively.

FIG. 2 summarizes the coexpression analysis of steroidal alkaloid-associated genes in Solanaceous plants. Shared homologs of coexpressed genes for ‘baits’ from tomato (SlGAME1 and SlGAME4) and potato (StSGT1 and StGAME4). Continuous (r-value >0.8) and dashed (r-value >0.63) lines connect coexpressed genes. *, located in the tomato or potato chromosome 7 cluster. St, Solanum tuberosum; Sl, S. lycopersicum. Background of gene names corresponds to bait they were found to be coexpressed with (legend above). SP, serine proteinase; PI, proteinase inhibitor; UPL, ubiquitin protein ligase; ELP, extensin-like protein; PK, protein kinase; SR, sterol reductase; RL, receptor-like.

FIG. 3 presents schematic map of genes identified in the duplicated genomic regions in in tomato and potato and their coexpression. Coexpression with GAME1 SGT1 (chromosome 7) and GAME4 (chromosome 12) as baits in either potato or tomato are presented in a form of a heatmap (Tables 5-8). Specific gene families are indicated by dark arrows while members of other gene families are in white arrows.

FIGS. 4A-4H shows functional analysis of tomato GAME genes. (FIG. 4A) GAME8-silenced transgenic (RNAi) leaves accumulated 22-(R)-hydroxycholesterol compared to wild type. (FIG. 4B) An array of cholestanol-type steroidal saponins (STSs) accumulates in GAME11 VIGS-silenced leaves. (FIG. 4C) An STS (m/z=753.4) accumulates in GAME12 VIGS-leaves. (FIG. 4D) Tomatidine, the steroidal alkaloid aglycone, accumulates in GAME1-silenced transgenic leaves. (FIGS. 4E to 411) Enzyme activity assays of the 4 recombinant tomato GAME glycosyltransferases.

FIGS. 5A-5D show solanine/chaconine levels in peels of tuber of potato plant lines with altered expression of GAME9 compared to wild type plants. Solanine (FIG. 5A) and chaconine (FIG. 5B) level in tubers of GAME9 silenced plant; Solanine (FIG. 5C) and chaconine (FIG. 5D) levels in tubers of GAME9 overexpressing plants.

FIG. 6 shows solanine/chaconine levels in leaves of potato plant lines with either silenced (RNAi) or overexpressed (OX) GAME9 compared to wild type plants.

FIG. 7 shows tomatine levels in leaves of tomato plant lines with either silenced (RNAi, line 5871) or overexpressed (OX, line 5879) GAME9 compared to wild type plants.

FIGS. 8A-8D show the effect of silencing of GAME11 dioxygenase in tomato. (FIG. 8A) α-tomatine levels in leaves (m/z=1034.5) (FIG. 8B) cholestanol-type steroidal saponins (STS) in leaves (m/z=1331.6, 1333.6, 1199.6, 1201.6 (major saponins)). (FIG. 8C) MS/MS spectrum of m/z=1331.6 (at 19.28 min.). (FIG. 8D) The fragmentation patterns of the saponin eluted at 19.28 min. and accumulating in GAME11-silenced leaves. Corresponding mass signals are marked with an asterisk on the MS/MS chromatogram in FIG. 8C.

FIGS. 9A-9E show metabolites extracted from GAME18-silenced mature green tomato fruit. Peaks of newly accumulating compounds corresponding to the γ-tomatine standard (m/z=740.5) (FIGS. 9A-C), and γ-tomatine pentoside (m/z=872.5) (FIGS. 9D-E) are shown.

FIGS. 10A-10D show the effect of silencing of GAME12 transaminase in tomato. (FIG. 10A) accumulation of a furastanol-type STS. (FIGS. 10B-10C) GAME12-silenced leaves accumulate an STS (m/z=753.4), while it exists in only minor quantities in WT leaf. (FIG. 10D) MS/MS spectrum of m/z=753.4 at 19.71 min. with interpretation of the fragments.

FIGS. 11A-11D show the effect silencing of GAME8 in tomato plants. GAME8-silenced leaves accumulated 22-(S) and -(R)-cholesterol (FIG. 11A). Chromatograms (mass range 172.5-173.5) acquired via EI-GC/MS, MS spectra and structures (tri-methyl-silyl derivatives) of the compounds are shown. Commercial standards of 22-(R)- (FIG. 11B) and 22-(S)-cholesterol (FIG. 11C) were used to verify the putative identification. (FIG. 11D) GAME8-silenced line accumulates both isomers in comparison to WT (Q).

FIG. 12 shows the phylogenetic tree of GAME genes in the plant CYP450 protein family. The numbers on the branches indicate the fraction of bootstrap iterations supporting each node.

FIG. 13 shows a proposed expanded biosynthetic pathway in Solanaceous plants from Cycloartenol (Part I), through Cholesterol (Part II), through Tomatidine (Part III), through Steroidal Glycoalkaloids including α-tomatine to Lycoperosides/Esculeoside (Part IV). Dashed arrows represent multiple enzymatic reactions in the pathway.

FIGS. 14A-14D show an overview of SGA biosynthesis in (A) tomato, (B) potato, and (C) eggplant. FIG. 14D shows a proposed portion of an SGA biosynthetic pathway in Solanaceous plants, from tomatidine to the production of tomatidine 3-O-glucuronide, wherein a GAME15 cellulose synthase like G enzyme catalyzes the addition of a glucuronic acid at the position of the hydroxyl group of the tomatidine.

FIGS. 15A-15C show major SGA levels in (A) leaves and (B) green fruit and (C) red fruit of wild type (non-transformed) and GAME15-RNAi tomato lines determined by LC-MS. #21, #22 and #23 are three independent GAME15-RNAi transgenic tomato lines. Values indicate means of three biological replicates ±standard error. Asterisks indicate significant changes from wild-type samples as calculated by a Student's t-test (*P-value <0.05; **P-value <0.01; ***P-value <0.001).

FIG. 16 shows levels of α-solanine and α-chaconine in leaves of GAME15-RNAi lines as determined by LC-MS. #1, #2 and #3 are three independent GAME15i transgenic potato lines. Values represent mean±standard error (n=3). Student's t-test was used to assess whether the transgenic lines significantly differ from wild-type plants: (*P-value <0.05; **P-value <0.01; ***P-value <0.001).

FIG. 17 shows the cholesterol content of tomato leaves derived from GAME15 silenced plants. Values represent mean of three biological replicates ±standard error. Asterisks indicate significant changes in leaves of the three independent transgenes (#21, #22 and #23) as compared to wild-type leaves (i.e. non-transformed) calculated by a Student's t-test (*P-value <0.05; **P-value <0.01; ***P-value <0.001). Epicholesterol was used as an internal standard in sample preparations and relative cholesterol level is expressed as ratios of cholesterol peak areas in sample compared to internal standard. The analysis was performed using GC-MS.

FIG. 18 are liquid chromatography-electrospray ionization-quadrupole-time of flight-mass spectrometry (LC-ESI-QTOF-MS) scans of spinach saponins. The upper scan shows the total ion chromatogram (TIC) of spinach leaf extract acquired in negative ion mode. Chromatogram fragment displayed and expanded below, shows saponins present in the sample. The lower scan presents an expanded region of the chromatogram with main saponins being numbered. The numbers represent different Yossosides as listed in Table 13 presented in Example 15.

FIGS. 19A-19P provide Tandem mass spectrometry (MS/MS) results and structures of Yossoside IV (FIGS. 19A-19D); of Yossoside Va (FIGS. 19E-19G); of Yossoside V (FIGS. 19I-19J); of Yossoside IX (FIGS. 19K-19M); and of Yossoside X (FIGS. 19N-19P). Details of the MS/MS figures showing results and structures are as follows: (FIG. 19A) Mass fragments originating from [M−H]11963.56=−m/z range for 300-1300; (FIG. 19B) Mass fragments originating from the cleavage of glucuronic acid residue—m/z range for 50-1900; (FIG. 19C) Structure of Yossoside IV, arrows indicate fragmentation patterns; (FIG. 19D) Table with assignments of MS/MS peaks originating from the fragmentation of glucuronic acid bound to MA; (FIG. 19E) Mass fragments originating from [M−H]1305.57190=−m/z range for 300-1400; (FIG. 19F) Mass fragments originating from the cleavage of glucuronic acid residue—m/z range for 50-1900; (FIG. 19G) Structure of Yossoside Va, arrows indicate fragmentation patterns. Position of acetyl group is putative.; (FIG. 19H) Mass fragments originating from [M−H]1305.5737=−m/z range for 300-1400; (FIG. 19I) Mass fragments originating from the cleavage of glucuronic acid residue—m/z range for 50-1900; (FIG. 19J) Structure of Yossoside V, arrows indicate fragmentation patterns; (FIG. 19K) Mass fragments originating from [M−H]1407.6060=−m/z range for 300-1500; (FIG. 19L) Mass fragments originating from the cleavage of glucuronic acid residue—m/z range for 50-1900; (FIG. 19M) Structure of Yossoside IX, arrows indicate fragmentation patterns. Structures and places of attachment of pentoses are putative.; (FIG. 19N) Mass fragments originating from [M−H]1437.6150=−m/z range for 300-1500; (FIG. 19O) Mass fragments originating from the cleavage of glucuronic acid residue—m/z range for 50-1900; and (FIG. 19P) Structure of Yossoside X, arrows indicate fragmentation patterns. Structure and attachment of pentose is putative.

FIGS. 20A-20C show the biosynthetic pathway of spinach saponins comprising co-expressed saponin β-amyrin synthase (SOAP) genes. (FIG. 20A) The complete biosynthetic pathway leading to the production of Yossoside V; an acetylated triterpenoid saponin containing glucuronic acid attached at C-20 position (highlighted in red). Each product of SOAP enzymes activity is highlighted in green. SOAP enzymes are those enzymes involved in saponin production in spinach. (FIG. 20B) Gene co-expression network. Large solid circles at SOAP1, SOAP2 and SoCYP716A268v2, represent baits for these genes, the central circle comprising solid dots depicts genes co-expressed with all the baits. Enlarged and annotated dots represent genes silenced by Virus Induced Gene Silencing (VIGS) in spinach. (FIG. 20C) Extracted ion chromatograms (EIC) of oleanolic acid, augustic acid and medicagenic acid [m/z=455.205 (OA); m/z=471.205 (AA); m/z=501.202 (MA); in negative ion mode] from plants transiently expressing SOAP1-4, compared to plants expressing SOAP1-20, SOAP1-2 alone and to control (plants infiltrated with Agrobacterium rhizogenes harboring empty vector—EV).

FIG. 21 presents important Heteronuclear Multiple Bond (HMBC) correlations of the hydrogens of Yossoside V. Black arrows represent HMBC correlations of the six methyl groups of Yossoside V. Red arrows represent HMBC correlations of other important hydrogens. Numbers correspond to carbon atoms in medicagenic acid and sugar moieties. For more details see Table 1. Table 1 presents spectral data summarized in tables that are split into two parts. The first part corresponds to atoms of medicagenic acid (aglycone part) while the second part corresponds to atoms of sugar moieties. The structures of Yossoside V and medicagenic acid 3-O-glucuronide with numbered atoms can be found in FIG. 21 and FIG. 35D, respectively. In Table 1: ax (axial) and eq (equatorial) depict protons in ring structures of aglycone and sugars.

TABLE 1
1H and 13C NMR spectral data for Yossoside V (3-O-[β-D-xylopyranosyl-(1−>3)-β-D-
glucuronopyranosyl]-28-O-[β-D-glucopyranosyl-(1−>4)-α-L-rhamnopyranosyl-(1−>2)-
4-acetyl-β-D-fucopyranosyl]-medicagenic acid) and for Medicagenic acid-3-O-glucuronide.
Yossoside V
aglycone part

| Atoms | ¹³C shift | ¹H shift |
|---|---|---|
| No. | (ppm) | (ppm) |
| 1 | 44.77 | 2.08 (eq) |
| | | 1.24 (ax) |
| 2 | 70.68 | 4.30 |
| 3 | 86.47 | 4.05 (ax) |
| 4 | 53.01 | |
| 5 | 53.01 | 1.57 (ax) |
| 6 | 21.53 | 1.61 |
| | | 1.16 |
| 7 | 33.65 | 1.35 (eq) |
| | | 1.49 (ax) |
| 8 | 40.96 | |
| 9 | 49.44 | 1.57 (ax) |
| 10 | 37.24 | |
| 11 | 24.68 | 1.93 (eq) |
| | | 1.99 (ax) |
| 12 | 123.51 | 5.26 |
| 13 | 144.57 | |
| 14 | 43.09 | |
| 15 | 28.96 | 1.18 (eq) |
| | | 1.58 (ax) |
| 16 | 23.92 | 1.60 (eq) |
| | | 2.04 (ax) |
| 17 | 48 | |
| 18 | 42.8 | 2.81 |
| 19 | 47.22 | 1.13 |
| | | 1.71 |
| 20 | 31.4 | |
| 21 | 34.77 | 1.22 (eq) |
| | | 1.38 (ax) |
| 22 | 33.01 | 1.56 (eq) |
| | | 1.75 (ax) |
| 23 | 182.07 | |
| 24 | 13.57 | 1.37 (ax) |
| 25 | 17.08 | 1.24 (ax) |
| 26 | 17.66 | 0.78 (ax) |
| 27 | 26.24 | 1.14 (ax) |
| 28 | 177.9 | |
| 29 | 33.45 | 0.89 (eq) |
| 30 | 24.1 | 0.92 (ax) |

sugars

| | | |
|---|---|---|
| β-D-Fuc | | |
| 1 | 94.94 | 5.38 (ax) |
| 2 | 74.58 | 3.74 (ax) |
| 3 | 74.57 | 3.88 (ax) |
| 4 | 75.13 | 5.06 (eq) |
| 5 | 71.06 | 3.83 (ax) |
| 6 | 16.37 | 1.06 |
| acetyl | 172.69 | |
| CO | | |
| Methyl | 20.78 | 2.14 |
| α-L-Rha | | |
| 1 | 101.48 | 5.33 (eq) |
| 2 | 71.66 | 3.90 (eq) |
| 3 | 72.04 | 3.83 (ax) |
| 4 | 84.52 | 3.53 (ax) |
| 5 | 68.74 | 3.80 (ax) |
| 6 | 18.28 | 1.32 |
| β-D-Glc | | |
| 1 | 106.4 | 4.47 (ax) |
| 2 | 76.14 | 3.18 (ax) |
| 3 | 78.28 | 3.34 (ax) |
| 4 | 71.46 | 3.27 (ax) |
| 5 | 78.09 | 3.25 (ax) |
| 6a | 62.85 | 3.66 |
| 6b | 62.85 | 3.84 |
| β-D-GICA | | |
| 1 | 104.79 | 4.39 |
| 2 | 68.93 | 3.81 |
| 3 | 70.94 | 3.81 |
| 4 | 74.31 | 3.43 |
| 5 | nd | nd |
| 6 | nd | |
| β-D-Xyl | | |
| 1 | 105.38 | 4.55 (ax) |
| 2 | 75.17 | 3.25 (ax) |
| 3 | 77.31 | 3.31 |
| 4 | 70.89 | 3.48 |
| 5a | 66.85 | 3.87 |
| 5b | 66.85 | 3.2 |

Medicagenic acid 3-O-glucuronide
aglycone part

| | | |
|---|---|---|
| 1 | 43.72 | 1.24 (ax) |
| | | 2.09 (eq) |
| 2 | 69 | 4.32 |
| 3 | 87.14 | 4.07 (ax) |
| 4 | 52.97 | |
| 5 | 51.73 | 1.62 (ax) |
| 6 | 20.49 | 1.29 |
| | | 1.62 |
| 7 | 32.44 | 1.24 (eq) |
| | | 1.56 (ax) |
| 8 | 39.44 | |
| 9 | 48.18 | 1.60 (ax) |
| 10 | 36.12 | |
| 11 | 23.31 | 1.89 (eq) |
| | | 2.02 (ax) |
| 12 | 121.05 | 5.22 |
| 13 | 145.36 | |
| 14 | 41.9 | |
| 15 | 27.84 | 0.95 (eq) |
| | | 1.87 (ax) |
| 16 | 23.2 | 1.57 (eq) |
| | | 1.88 (ax) |
| 17 | nd | |
| 18 | 42.07 | 2.90 |
| 19 | 46.73 | 1.08 |
| | | 1.65 (eq) |
| 20 | 30.43 | |
| 21 | 34.12 | 1.12 (eq) |
| | | 1.34 (ax) |
| 22 | 32.94 | 1.49 (eq) |
| | | 1.72 (ax) |
| 23 | 184.8 | |
| 24 | 13.46 | 1.36 (ax) |
| 25 | 15.8 | 1.26 (ax) |
| 26 | 16.97 | 0.88 (ax) |
| 27 | 25.15 | 1.14 (ax) |
| 28 | nd | |
| 29 | 32.56 | 0.87 (eq) |
| 30 | 22.81 | 0.95 (ax) |

glucuronic acid

| | | |
|---|---|---|
| β-D-GICA | | |
| 1 | 103.14 | 4.41 (ax) |
| 2 | 74.02 | 3.27 (ax) |
| 3 | 75.95 | 3.40 (ax) |
| 4 | 74.52 | 3.60 (ax) |
| 5 | 72.09 | 3.39 (ax) |
| 6 | 175.6 | |

FIGS. 22A and 22B show that the genes involved in first steps of aglycone biosynthesis are located in close vicinity on the chromosome. (FIG. 22A) Schematic representation of the chromosomal region with genes involved in oleanolic acid biosynthesis. Semitransparent gray boxes contain SOAP1 (bAS) (SEQ ID NO: 422), CYP716A268 (SOAP2; SEQ ID NO: 46) and its truncated duplication CYP716A268v2 (SEQ ID NO: 47). Genes marked in dark grey are not directly involved in the biosynthesis of triterpenoid saponins. (FIG. 22B) Expression pattern of SOAP1, SOAP2 and CYP716A268v2 among five spinach transcriptomes; S04WOLLD—mature leaf from four week old plant grown in long day; SO8WOLSL—mature leaf from eight week old plant grown in short day; SO4WYLLD—young leaf from four week old plant grown in long day; SO8WOLSL—young leaf from eight week old plant grown in short day; SO4WFBLD—flower bud from four week old plant grown in long day. For details see Materials and Methods.

FIGS. 23A-23D present data providing functional characterization of SOAP1 (bAS) and SOAP2 (CYP7123A2238) by silencing (VIGS) in spinach. (FIG. 23A) Chlorotic phenotype observed in plant with silenced magnesium-chelatase subunit H (CHLH). CHLH was used as a control and a marker of VIGS gene silencing. In general, this gene is not directly related to SOAP genes and saponin biosynthesis. EV stands for empty vector and represents a control leaf. (FIG. 23B) Real-time qPCR analysis of SOAP1 and SOAP2 expression in silenced plants. Values (±SD) represent mean of three independent biological experiments. Statistically significant differences compared with control plants (CHLH silenced) are indicated; ***P<0.001. (FIG. 23C) Aligned gas chromatography-mass spectrometry (GC-MS) extracted ion chromatograms (EICs) of β-amyrin [m/z=218, main fragment] from the plants with silenced SOAP1 or SOAP2 compared to control (CHLH). β-Amyrin can be detected only in plants with silenced SOAP2 where its further conversion is blocked. (FIG. 23D) Aligned liquid chromatography-mass spectrometry (LC-MS) EICs of Yossoside IV [m/z=12233.23, (−)-mode] from plants with silenced SOAP1 or SOAP2 compared to control (CHLH). Content of Yossoside IV was reduced to 40 and 30% in SOAP1 and SOAP2 silenced lines, respectively.

FIGS. 24A and 24B present data providing functional characterization of SOAP1 (bAS; SEQ ID NO: 45) and SOAP2 (CYP2416A268; SEQ ID NO: 46) by heterologous expression in N. benthamiana. (FIG. 24A) Aligned GC-MS extracted ion chromatograms (EICs) of β-amyrin [m/z=218, main fragment] from the transient expression of SOAP1 compared to empty vector (EV) alone and an authentic standard. Only chromatograms from in planta experiments are to scale. (FIG. 24B) Aligned GC-MS EICs of oleanolic acid [m/z=203, main fragment] from the transient expression of SOAP1 and SOAP2 compared to empty vector (EV) alone and an authentic standard. Only chromatograms from in planta experiments are to scale. For details of GC-MS analysis see Materials and Methods.

FIG. 25 presents data showing liquid chromatography-mass spectroscopy (LC-MS) analysis of selected saponins in spinach with (VIGS) silenced cytochrome P450 candidate genes. Relative content (normalized peak area) of selected saponin compounds in plants with silenced SOAP3 (CYP72A655; SEQ ID NO: 51), SOAP4 (CYP72A654; SEQ ID NO: 53), and additional three other CYPs (p450 cytochrome genes); for accession numbers see Table 11. Values (±SD) represent mean of three independent biological experiments. Statistically significant differences compared with control plants (CHLH silenced) are indicated; *P<0.05, **P<0.01.

FIGS. 26A-26L present characterization of saponins accumulating in spinach following silencing of cytochrome P450 genes (VIGS). (FIGS. 26A, 26D, 26G, and 26J) Relative content (normalized peak area) of selected triterpenoid saponin compounds in spinach leaves with silenced SOAP3 (CYP72A655; SEQ ID NO: 51), SOAP4 (CYP72A654; SEQ ID NO: 53) and additional three cytochrome p450 genes; for accession numbers see Table11. Values (±SD) represent mean of three independent biological experiments. Statistically significant differences compared with control plants (CHLH silenced) are indicated; *P<0.05, **P<0.01. AA—augustic acid (FIGS. 26B, 26E, 26H, and 26K) Tandem mass spectroscopy (MS/MS) spectra with marked main fragments. (FIGS. 26C, 26F, 26I, and 26L) Putative structures of saponin compounds based on accurate mass measurement, fragmentation patterns, and literature. For more details see Table 16.

FIG. 27 presents GC-MS analysis of saponins' aglycones in spinach plants with silenced CYPs P450. EICs of triterpenoid aglycones [m/z=203 and 262; common fragments for all amyrin-type aglycones] released from the saponins accumulated in plants with silenced SOAP3 alone, SOAP4 alone, and CYP2 compared to control (CHLH silenced) and authentic standards of oleanolic and medicagenic acid. Scale of each EIC was normalized to the most abundant signal. Numbers from 1-7 represent names of detected compounds, 1—oleanolic acid; 2—hederagenin; 3—augustic acid; 4—gypsogenin; 5—bayogenin; 6—unknown; 7—medicagenic acid, respectively.

FIGS. 28A-28C presents the analysis and putative characterization of aglycones in spinach with silenced CYPs P450. (FIG. 28A) Relative content of detected sapogenins in plants with silenced SOAP3, SOAP4, CYP2 and CHLH as control. (FIG. 28B) Structures of characterized aglycones. Numbers from 1-7 represent names of detected compounds, 1—oleanolic acid; 2—hederagenin; 3—augustic acid; 4—gypsogenin; 5—bayogenin; 6—unknown; 7—medicagenic acid, respectively. (FIG. 28C) Fragmentation patterns of described compound and comparison with fragmentation patterns of authentic standards of oleanolic acid and medicagenic acid.

FIG. 29 presents LC-MS analysis of selected saponins in spinach with silenced (VIGS) for glycosyltransferase genes. Relative content (normalized peak area) of selected saponin compounds in plants with silenced SOAP6-9 (SEQ ID NOs: 55, 57, 59, and 61, respectively), SoGT1 (SEQ ID NO: 117 [gene] and SEQ ID NO: 118 [polypeptide]) and SoGT2 (SEQ ID NO: 119 [gene] and SEQ ID NO: 120 [polypeptide]); for accession numbers see Table 11. Sequences used for the glycosyl transferases are provided below.

Spinach GT1 nucleic acid gene sequence
is set forth as follows:
(SEQ ID NO: 117)
atggccgcctcaaacaaagagcaaagcaagctacatattgctatg
tttccttggtttgcatatggtcatataaacccattcatccagctc
tctaacaagctttcctcccatggcatccaaatctctttcttctca
ataccaggaaacattgatcgtatcaaatcctcccttaatctctca
cctcccaaccagctcatccccctcactattcccccaactgaagga
ctctctcccaactttgacagcagctctgaagtaacacctcaaact
gctcagcttctcacactagcacttgatcaaatgcagccccaagtc
aaagctctattccctcacccccagccacaagttatcctctttgat
tttgcatatcactggcttccctcagtagcttctgaactaggcatc
aaagctgttcatttcaatacattcccagctgttatcaattcatat
ctcactgtcccttcaagaatgactgatccaaataaaccaccgaca
tttgaggacttgaagaaccctcctcaaggctatcccaaaacctca
accgcctcagtgaaaaccttcgaagctcaagattacctattcctt
ttcaagagtttcgatggcggaccgtgccatttcgaaaagatattg
gcattcacaaacagctgtgatgctatactttacaggacctgcaat
gaaatagaaggtccattcatagattacttcaagacccaaataaat
aaaccactgcttttagctggcccaagtgttcctctaccaccctct
ggtgaactggatgaaaaatgggagatgtggttaggtaaatttcct
gaaaagtcagtcatatactgcagcttcggaagcgagacatacttg
aatgatgctcagattcaggagcttacacttgggttggagctcact
ggtctgccctttatcttggttttgaattttggaacaagtaacagc
accgatgcccacaataagctagaagcatcattaccagaaggattt
agagagagaatcaaagacaggggcgttctgcatacaggatgggtg
caacagcaaaacattttagcacacagaagcataggatgctttctt
actcacgcagggttcagctctgtaatagagggtattgtgaatgac
tgtcaattagcatttctacctctaaaggctgaccagtttatgatc
gctaagctatttagtggggatctgaaagcaggggtggaggtaaat
cgaagagatgaagatgggtcttttgccaaagaagatattttcgaa
gcaataaagacgatcatggtggatactgataaagaaccaagtaga
tccatcagggagaatcatagcaattggagaaagtttttgatgaat
aaggagattgaagctagttatattgcaaatttagctcatgaactc
aaggcattggttcaaaaagcttag.

Spinach GT1 nucleic acid gene sequence is set forth as follows:

(SEQ ID NO: 118)
MAASNKEQSKLHIAMFPWFAYGHINPFIQLSNKLSSHGIQISFFS
IPGNIDRIKSSLNLSPPNQLIPLTIPPTEGLSPNFDSSSEVTPQT
AQLLTLALDQMQPQVKALFPHPQPQVILFDFAYHWLPSVASELGI
KAVHFNTFPAVINSYLTVPSRMTDPNKPPTFEDLKNPPQGYPKTS
TASVKTFEAQDYLFLFKSFDGGPCHFEKILAFTNSCDAILYRTCN
EIEGPFIDYFKTQINKPLLLAGPSVPLPPSGELDEKWEMWLGKFP
EKSVIYCSFGSETYLNDAQIQELTLGLELTGLPFILVLNFGTSNS
TDAHNKLEASLPEGFRERIKDRGVLHTGWVQQQNILAHRSIGCFL
THAGFSSVIEGIVNDCQLAFLPLKADQFMIAKLFSGDLKAGVEVN
RRDEDGSFAKEDIFEAIKTIMVDTDKEPSRSIRENHSNWRKFLMN
KEIEASYIANLAHELKALVQKA.

Spinach GT2 nucleic acid gene sequence is set forth as follows:

(SEQ ID NO: 119)
atgtgtgacgacaaaaaatcatctgttttgagcatagcattttatccgtggtttgctcttggtcacct
tacttcatttctccgattagccaacaaacttgcacaaaatggtcacaatgtgtcctattttatcccaa
ctaatacattacctagattacttcctcacaaccattaccctggccaccttactttcatccccgtcacc
gtcccacccgttgacggcctccctctcagagccgagaccaccaacgatgtcccctcctcggctataca
ccttcttatgactgccatggatttgacccgtgacactatcgaggcccatttggttagtatcaaacccg
atgttgttttctacgactttgcttattggattcccgatctagcccgaaaacacgggttcaagtcagta
ctctacattacatcctatatagcaagatgtgcttattttgcccccgatttgaagtcgggtcatcagtc
cactggggccgaaattattgcgccaccaccgggttttccgtctcagcatttccggatgcaagcacacg
aggctgagactgtggcagacgtaggtaaagagcaagatggattacaaggtataactatttctgaaagg
atgcgcattgcttttggaaaatgcgacgcaattggagtaaagagttgtaaggagatggaaaaggtgta
tattgactactgtgagaagatatttggtaagtctgtactactagcaggtcctatggtccctaaaaccc
catcttccaaacttgatgaatattttgatggttggcttacgggttttggtgctgctactgtgatttat
tgtgcatttgggagtgaatgtgttctcgaaattaaccaatttcaacaacttcttcttggactagagct
cacaggaaggccatttttggtggccatgaagccgcctaagaagtatgaaacaatagagtcggccttac
cagaagggtttgagaagagaacaaaaggaaggggaatcgtacatgagggttgggtgcagcaacaactg
atattgcaacatccatcagtaggatgtttcataactcattgtggagttgggtctctttcggaagctat
ggtcagcaaatgtcaagtagtgttgatgcctcaagctgtagaccaattcatcaatgcgaggatgatga
gtttagagttgaagattggggttgaggttgagaagagagaagatgatggtttgttcacaaaggaggct
gtgcataaggcggtctctttggtgatggaggaagaaagtgaagtcgcaaaagagatgagggtaagtca
tgataaatggagagaattcttattacaggaaggtcttgaggattcttatatcagtagcttcattcaga
gtctacgacagttaacgattggatga.

Spinach GT2 amino acid gene polypeptide is set forth as follows:

(SEQ ID NO: 120)
MCDDKKSSVLSIAFYPWFALGHLTSFLRLANKLAQNGHNVSYFIPTNTLPRLLPHNHYPG
HLTFIPVTVPPVDGLPLRAETTNDVPSSAIHLLMTAMDLTRDTIEAHLVSIKPDVVFYDFA
YWIPDLARKHGFKSVLYITSYIARCAYFAPDLKSGHQSTGAEIIAPPPGFPSQHFRMQAHE
AETVADVGKEQDGLQGITISERMRIAFGKCDAIGVKSCKEMEKVYIDYCEKIFGKSVLLA
GPMVPKTPSSKLDEYFDGWLTGFGAATVIYCAFGSECVLEINQFQQLLLGLELTGRPFLV
AMKPPKKYETIESALPEGFEKRTKGRGIVHEGWVQQQLILQHPSVGCFITHCGVGSLSEA
MVSKCQVVLMPQAVDQFINARMMSLELKIGVEVEKREDDGLFTKEAVHKAVSLVMEEE
SEVAKEMRVSHDKWREFLLQEGLEDSYISSFIQSLRQLTIG

Values (±SD) represent mean of three independent biological experiments. Statistically significant differences compared with control plants (CHLH silenced) are indicated; *P<0.05, **P <0.01, ***P<0.001.

FIGS. 30A-30C present data showing putative characterization of saponins accumulated in spinach with silenced (VIGS) glycosyltransferases. (FIG. 30A) Relative content of selected compounds in plants with silenced SOAP6-SOAP9 (SEQ ID NO: 55, 57, 59, and 61, respectfully) and other two GTs. Values (±SD) represent mean of three independent biological experiments. Statistically significant differences compared with control plants (CHLH silenced) are indicated; **P <0.01, ***P<0.001. (FIG. 30B) MS/MS spectra with the main fragments marked. (FIG. 30C) Putative structures of accumulated saponins are based on accurate mass measurement and fragmentation patterns.

FIGS. 31A-31F present characterization of SOAP10 acetyltransferase in planta (spinach) and in vitro. (FIG. 31A) LC-MS analysis of samples from spinach plants with VIGS silenced SOAP10 (red trace) or CHLH (black trace) as control. Blue and yellow regions of the 19n chromatogram contain desacetyl and acetylated saponins, respectively. Silencing of SOAP10 in spinach leaves resulted in decreased production of acetylated saponins and increased accumulation of desacetyl counterparts. (FIG. 31B) Relative content of selected desacetyl saponins (Yossoside IV and XII) and their acetylated counterparts (Yossoside V and VII) in spinach plants with VIGS silenced acyltransferases and CHLH as control. The ordinate axis is in log 10 scale. (FIG. 31C) Ratio of desacetyl to acetylated saponins in VIGS silenced spinach plants. Silencing of SOAP10 resulted in increased accumulation of desacetyl saponins. (FIGS. 31D, 31E, and 31F) SOAP10 recombinant protein activity in vitro. EICs of desacetyl saponins (FIG. 31D—m/z 1101.51; FIG. 31E—m/z 1263.57; FIG. 31F—m/z 1395.61) and acetylated counterparts (FIG. 31D—m/z 1313.52; FIG. 31E—m/z 1305.58; FIG. 31F—m/z 3137.62) in: substrate (bottom trace in each of FIGS. 31D, 31E, and 31F)—purified fraction of desacetyl saponins from spinach; in vitro enzymatic assay with recombinant SOAP10 incubated with substrate in presence of acetyl-CoA (middle trace in each of FIGS. 31D, 31E, and 31F); and in extract of spinach leaves (upper trace in each of FIGS. 31D, 31E, and 31F) for comparison. Dotted line separates regions on chromatogram with desacetyl and acetylated saponins on the left- and right-hand side, respectively. “Δm/z=42.01” depicts difference in molecular weight of the compounds resulting from acetylation. (SoAT1 (SEQ ID NO: 109 [gene] and SEQ ID NO: 113 [polypeptide]), SoAT2 (SEQ ID NO: 110 [gene] and SEQ ID NO: 131 [polypeptide]), SoAT3 (SEQ ID NO: 111 [gene] and SEQ ID NO: 115 [polypeptide]), and SoAT4 (SEQ ID NO: 112 [gene] and SEQ ID NO: 116 [polypeptide]), for functional activities of SoAT1-SoAT4 see Table 15).

FIGS. 32A-32D present data from the expression of SOAPs 1-4 and 6-10 in N. benthamiana. (FIG. 32A) EIC of medicagenic acid and its (poly)glycosylated derivatives [m/z=501.32 (MA); m/z=663.38 (MA+hex); m/z=825.43 (MA+2hex); m/z=987.48 (MA+3hex); m/z=1149.53 (MA+4hex) in negative ion mode] from plants transiently expressing either SOAP1-4 alone or all nine SOAPs without SOAP5 (SEQ ID NOs: 65 or 93 [gene], SEQ ID NO: 66 [polypeptide]), compared to control. Inset shows region of the chromatogram magnified 20 times. (FIGS. 32B, 32C, and 32D) show MSE spectra of mono-, bis- and trishexosylmedicagenic acid, respectively. Arrows represent losses of hexosyl moieties.

FIGS. 33A-33F present technical analysis of Cellulose Synthase Like G gene (SOAP5; SEQ ID NOs: 65 or 93 [gene], SEQ ID NO: 66 [polypeptide]). The SOAP5 encoded enzyme attaches glucuronic acid to the triterpenoid aglycone and plays a key role in spinach saponin biosynthetic pathway. (FIG. 33A) Analysis of SOAP5 relative expression in spinach leaves after Virus Induced Gene Silencing (VIGs). Presented values were obtained from 4 independent biological replicates. Expression of SOAP5 was compared to the control (plants with silenced magnesium chelatase subunit H alone—CHLH). (FIG. 33B) Medicagenic acid accumulation in spinach leaves with silenced SOAP5 and other glycosyltransferases compared to control (CHLH). Significant differences are indicated, ***P<0.001. (FIG. 33C) EIC of medicagenic acid 3-O-glucuronide [m/z=677.35 (MA-3-GlcA), in negative ion mode] from plants transiently expressing SOAP1-5, compared to plants expressing SOAP1-4 alone and to control (empty vector—EV). (FIG. 33D) EIC of MA-3-GlcA [m/z=677.35 in negative ion mode] from yeast expressing SOAP1-5+SoUGD1 compared to yeast expressing SOAP1-5, yeast controls (SOAP1-4+SoUGD1; SOAP5 alone and yeast cells transformed with EV). (FIG. 33E) Schematic representation of SOAP5 activity in spinach. SOAP5 attaches glucuronic acid (highlighted in red) to medicagenic acid at position C-3. (FIG. 33F) Spinach saponin biosynthetic pathway reconstitution in N. benthamiana. EIC of Yossoside V [m/z=1305.57 in negative ion mode] in samples from N. benthamiana leaves transiently expressing all the ten SOAP genes compared to control plants (EV) and to spinach leaf extract.

FIGS. 34A-34B present the data showing the results of silencing SOAP5 in spinach (VIGS). (FIG. 34A) EIC of medicagenic acid [m/z=501.3, in negative ion mode] from plants with silenced SOAP5 (SEQ ID NOs: 65 or 93), compared to control (CHLH silenced) and to wild type (WT). Only chromatograms from inplanta experiments are to scale. (FIG. 34B) Tandem mass spectroscopy (MS/MS) of medicagenic acid [25V, positive ion mode] accumulated in the plant compared to the authentic standard. MA—medicagenic acid.

FIGS. 35A-35C present data showing transient expression of SOAP5 (SEQ ID NOS: 65 OR 93) in N. benthamiana. (FIG. 35A) EIC of medicagenic acid and medicagenic acid 3-glucuronide [m/z=501.32 (MA); m/z=677.35 (MA-3-GlcA), in negative ion mode] from plants transiently expressing SOAPs1-5 (SEQ ID NOs: 45, 46, 51, 53, and 65, respectively), compared to plants expressing SOAPs1-4 alone (SEQ ID NOs: 45, 46, 51, and 53, respectively), SOAP5 alone (SEQ ID NOS: 65 OR 93) and to control (empty vector-EV). Chromatograms are to scale. (FIG. 35B) Structure of MA-3-GlcA with marked fragmentation routes (FIG. 35C) MS/MS of MA-3-GlcA [45V, negative ion mode], arrow indicates loss of glucuronic acid moiety. (FIG. 35D) shows important HMBC correlations of the hydrogens of medicagenic acid 3-O-glucuronide (MA-3-GlcA). Black arrows represent HMBC correlations of the six methyl groups of MA-3-GlcA. Red arrows represent HMBC correlations of other (selected) hydrogens of MA-3-GlcA. Numbers correspond to carbon atoms in medicagenic acid and glucuronic acid moiety. For more details see Table 1.

FIGS. 36A-36F present the substrate specificity of SOAP5 (SEQ ID NO: 66) (See Table 16). EIC of oleanolic acid 3-GlcA (FIG. 36A), augustic acid 3-GlcA (FIG. 36B), and gypsogenic acid 3-GlcA (FIG. 36C) [m/z=631.38 (OA-3-GlcA); m/z=647.38 (AA-3-GlcA); m/z=661.35 (GA-3-GlcA), in negative ion mode] from plants transiently expressing SOAP1/2/5 (FIG. 36A), SOAP1/2/3/5 (FIG. 36B), SOAP1/2/4/5 (FIG. 36C) compared to plants expressing same set of SOAPs without SOAP5 and to control (EV). Chromatograms are to scale. (FIGS. 36D, 36E, and 36F show tandem mass spectroscopy (MS/MS) of glucuronate derivatives from FIGS. 36A, 36B, and 36C, respectively, [40V, negative ion mode], arrows indicate loss of glucuronic acid (Δm/z=176.03; GlcA-H2O).

FIGS. 37A-37E show the products of SOAPs activity in N. benthamiana. EICs of Yossoside I (m/z=823.42) (FIG. 37A), Yossoside II (m/z=969.27) (FIG. 37B), Yossoside III (m/z=1131.52) (FIG. 37C), Yossoside IV (m/z=1263.57) (FIG. 37D) and Yossoside V (m/z=1305.58) (FIG. 37E) from plants expressing transiently combinations of SOAP genes indicated on each chromatogram. For MS/MS spectra see FIG. 40B.

FIGS. 38A-38E shows mass spectroscopy (MS) based identification of the main products of SOAPs activity in N. benthamiana. (FIG. 38A) MS/MS of Yossoside I (Compound 7)—product of SOAP6 activity on MA-3-GlcA. (FIG. 38B) MS/MS of Yossoside II (Compound 8)—product of SOAP7 activity on Yossoside I. (FIG. 38C) MS/MS of Yossoside III (Compound 9)—product of SOAP8 activity on Yossoside II. (FIG. 38D) MS/MS of Yossoside IV (Compound 10)—product of SOAP9 activity on Yossoside III. (FIG. 38E) MS/MS of Yossoside V (Compound 11)—product of SOAP10 activity on Yossoside IV.

FIGS. 39A-39C present the results of SOAP9 activity in N. benthamiana. (FIG. 39A) EIC of medicagenic acid 3-GlcA-Xyl [m/z=809.40 in negative ion mode] from plants transiently expressing SOAP1-5+SOAP9 compared to plant expressing SOAP1-5 alone and to control (EV). (See Table 16) (FIG. 39B) MS/MS of 809.4 (45V), arrow indicates loss of glcA-xyl moiety (Δm/z=308.07). (FIG. 39C) Structure of the SOAP9 product (Compound 10) with fragmentation patterns indicated by arrows.

FIGS. 40A-40B show the results of expression of SOAP5 in yeast. (FIG. 40A) EIC of medicagenic acid and medicagenic acid 3-GlcA [m/z=501.32 (MA); m/z=677.35 (MA-3-GlcA), in negative ion mode] from yeast expressing SOAP1-5+UGD1 (SEQ ID NO: 74 [gene] and SEQ ID NO: 75 [protein]) compared to plants expressing SOAP1-5, yeast controls and to medicagenic acid authentic standard. Chromatograms are to scale. (FIG. 40B) MS/MS of medicagenic acid 3-GlcA from yeast cells [45 V, negative ion mode], arrows indicate loss of glucuronic acid.

FIGS. 41A-41F show SOAP5 substrate specificity in yeast cells. EIC of oleanolic acid 3-GlcA (FIG. 41A), gypsogenic acid 3-GlcA (FIG. 41B) and Bayogenin 3-GlcA (FIG. 41C) [m/z=631.38 (OA-3-GlcA); m/z=661.35 (GA-3-GlcA); m/z=663.37 (Bayogenin-3-GlcA), in negative ion mode] from yeast expressing SOAP1-5+UGD1 compared to cells expressing SOAP1-4+UGD alone, SOAP1-5 alone, SOAP1-4, SOAP1-2, SOAP5 alone, and to control (EV). Chromatograms are to scale. (FIGS. 41D, 41E, and 41F) MS/MS of glucuronate derivarives from A, B and C [45V, negative ion mode], arrows indicate loss of glucuronic acid (Δm/z=176.03; GlcA-H2O).

FIGS. 42A-42F show Glucuronosyltransferase activity of Cellulose Synthase Like G (CSLG) enzymes is conserved among species belonging to phylogenetically distant orders. (FIG. 42A) The phylogenetic tree depicts close evolutionary relationship of SOAP5 from spinach with some CSLG proteins from Caryophyllales, Malvales, Apiales, Fabales, and Solanales. The tree consists of proteins belonging to Cellulose Synthase (CESA) and several families of Cellulose Synthase Like enzymes (CSLA, CSLB, CSLE and CSLG). Dotted line encircles clades with CSLG proteins, while blown up region corresponds to CSLG closely related to spinach SOAP5 including GAME15 and CSLG in the Fabales ordera. (FIG. 42B) Medicagenic acid is glucuronidated by CSLG from many species. EIC of MA-3-GlcA [m/z=677.35 in negative ion mode] of samples from N. benthamiana leaves transiently expressing SOAP1-4 combined with CSLG proteins from spinach (SOAP5; SEQ ID NO: 66), Chenopodium quinoa (CqCSL; SEQ ID NO: 96), Beta vulgaris (BvCSL; SEQ ID NO: 94), Medicago sativa (MsCSL; SEQ ID NO: 98), Glycine max (GmCSL; SEQ ID NO: 100), Glycyrrhiza uralensis (GuCSL; SEQ ID NO: 81 or SEQ ID NO: 102) and Lotus japonicus (LjCSL; SEQ ID NO: 104), compared to plants expressing SOAP1-4 alone and to control (EV). (FIG. 42C) Cellulose synthase like G proteins are key enzymes in the biosynthesis of triterpenoid saponins in multiple plant species. β-Amyrin (in the center) is oxidized by cytochromes P450 (doubled circle around β-Amyrin) giving rise to multiple aglycons (1—bayogenin; 2—serjanic acid; 3—oleanolic acid; 4—medicagenic acid; 5—glycyrrhetinic acid; 6—soyasapogenol A; 7—soyasapogenol B) that are decorated by CSLGs (double circle around aglycons 1-7) and other glycosyltransferases giving rise to triterpenoid saponins (8—bayogenin-hexA-hex-hex (M. sativa); 9—serjanic acid-hexA-hex (Chenopodium quinoa (C. quinoa)); 10—betavulgaroside IV (B. vulgaris); 11—yossoside V (S. oleracea); 12—glycyrrhizin (G. uralensis); 13—soyasapogenol A—hexA-hex-pent (L. japonicus); 14—soyasaponin VI (G. max)). (FIG. 42D) Glycyrrhizin biosynthetic pathway in G. uralensis. Highlighted functional groups are the result of the activity of specified enzymes. (FIG. 42E) Extracted ion chromatogram (EIC) measurements of glycyrrhetinic acid 3-O-glucuronide and glycyrrhizin [m/z 645.36 and m/z 821.40 in negative ion mode] from N. benthamiana leaf samples transiently expressing five genes from glycyrrhizin biosynthetic pathway (bAS (SEQ ID NO: 45)+CYP88D6 (SEQ ID NO: 76)+CYP72A154 (SEQ ID NO: 78)+GuCSL (SEQ ID NO: 80 or SEQ ID NO: 103), and UGT73P12 (SEQ ID NO: 84)) compared to samples expressing four genes (-UGT73P12; (SEQ ID NO: 84)) and to control (bAS (SEQ ID NO: 45)+CYP88D6 (SEQ ID NO: 76)+CYP72A154 (SEQ ID NO: 78)). (FIG. 42F) Presents a phylogenetic tree of Cellulose Synthase Like (CSL) polypeptides with special emphasis on Cellulose Synthaes Like G (CSLG). The lower shaded background region consists of CSLG enzymes from many plant orders (i.e., Carylophyllales, Fabales, Apiales, Malvales, and Solanales). Within the CSLG enzymes (shaded background), related subclades include the subclade containing CSLG enzymes (in bold) with proven glucuronosyltransferase activity towards triterpenoid aglycones—CSLG from soybean, licorice, Lotus japonicus, alfalfa, red beet, quinoa, and spinach, and the subclade consisting of CSLGs from Solanales (Solanum tuberosum, potato; Solanum lycopersicum, tomato; Solanum dulcamara, bittersweet) and other orders; enzymes in bold are involved in steroidal alkaloid and steroidal saponin biosynthesis.

FIGS. 43A-43C shows LC-ESI-QTOF-MS analysis of saponins in quinoa, beetroot and alfalfa. Total Ion Chromatogram (TIC) of 80% methanolic extract of (FIG. 43A) one-month old quinoa plants (Chenopodium quinoa var. Read Head), (FIG. 43B) one-month old beetroot of (Beta vulgaris var. Bohan) and (FIG. 43C) 2-week-old seedlings of alfalfa (Medicago sativa). Semitransparent rectangles mark chromatogram region with saponins. For putative characterization see Table 12.

FIGS. 44A-44C show virus induced gene silencing (VIGS) of CSLG in red beet (FIG. 44A) Virus induced gene silencing in red beet; EV—empty vector, CHLH—magnesium chelatase subunit H, BvCSL—beetroot cellulose synthase like G (SEQ ID NO: 95 [gene]; VIGs silencing sequence SEQ ID NO: 107). (FIG. 44B) Analysis of relative content (peak area) of triterpenoid saponins and oleanolic acid in red beet plants with silenced BvCSL. The ordinate axis is in log₁₀ scale. Values represent mean of three independent biological experiments. Statistically significant differences compared with control plants (CHLH silenced alone) are indicated; *P<0.05, ***P<0.001. (FIG. 44C) EIC of oleanolic acid [m/z=455.35, negative ion mode] from plants with silenced BvCSL compared to control (CHLH and WT) and authentic standard. (For metabolite identification see Table 12 M. sativa.)

FIGS. 45A-45B show silencing of CSLG in alfalfa hairyroots. (FIG. 45A) EIC of representative saponins containing glucuronic acid attached to the aglycone from hairyroot cultures with silenced Medicago sativa Cellulose Synthase Like (MsCSL) compared to control (EV). (FIG. 45B) Analysis of triterpenoid saponins in alfalfa hairyroots with silenced MsCSL. The ordinate axis is in log₁₀ scale. Values represent mean of five independent biological experiments. Statistically significant differences compared with control (roots transformed with EV alone) are indicated; *P<0.05, ***P<0.01, ***P<0.001. (asRNA—antisense RNA) The asRNA sequence used is set forth here:

(SEQ ID NO: 108)
CTACCCACTCTTCCGCTTCATAGATATAATCCCCTCCACAATAGGATGACTTAGAGCA
AGTATATAGATAATGAGGAAAAGTTGACCAAACATTTCATCAAAATCCCTCACATTG
AGTAGTCTCCATAAACCACCAAAGAAGCAAACAATATTTATTGTGAGTAACACAATC
ACTGGTGCCATGAGCAATGCTGCACCATCAAAATTAAACTTACCCTGCTCATATTTCT
TTAGCTTATCTTTGTCAATTGCTTTGTCCGATAAAGTGAATTTTGCCTTGTTCAACCCA
AACTTTTTTTTCGCTGTTTCTATAATTGCGAAAACGCACCCAATTGATTTTACAATCCC
ACTTCTTTGTTCATTCCACCAAATCATCGAGGAGCCACCAGTTGAAATTACCTCAATA
TAATGATGAATTTGACTGGATAGATACAATACTGTAAACACTATAAACCAAGGATCT
GTAACCTTTGGATATACAGGTATTCCCTTCAAGAAGCAAATCTGAGGAACGAATCCA
TATAAGATGTAGGCTGTTGCAAATTGGGTTGTGCTTACCAAGTAACAAAAAGTTAAA
CAATGAATAGTGGGCAATCTTGAAAGGCCATAAGTGAATGGGCTATATTTAGAGATT
GCAAGCAACAAAAGTTCAGATGACCACTTTATCGGTTGAATCAATCCCTCTTTCATGT
CAGTTGGAGCACATCCTAAGAAACATGGTCTTTTAGGGTAAAGATAAGTTGATCTCC
ATCCTCTACAATGGAGGAGATAGCCAGTAACGGTACTCTCAAGTTTTATAGCATACG
AGAATCCCACCTCATTACCCCAATTTGTGTTTCTCTCATAGGAACAAGAAGCCACTTC
ACATGCTTCTTGTAAAATTACATCTCTTGAAATATTCTGCTTCTTAGTTTGTTGACCAC
GTAACGCTTTTAGTGATTCTACATACATGTTAGACTTGCCAAAGTTGTATAGAGCATC
AAGTAGATAGTCCCCTTTTTGGTTTGGACTTCCAAAGAGTAATGCACTTCTACTTATA
TAATTTCCACTGCCAGTTAGACCTGGACCTCTTAATCCATCCATTCCCTTCCACTTTGT
CGTAAAAGCAGTCCTAGTCTGACTATCATATATGTCTTTCTTGCTAAGGTTGTGAAAC
ATTTGAGGGAATTGAACAAAAGCAACATCTTTAGAGGTTTCAGGATCAAGAAAAAAG
CACATGGATTGTTTGGCTGATGATGCATCATTACAATTCATATCACAATCTACGACAA
GTACATAAGGTCCATTGCTGATTAGCCCTGACACCCTAAGCAATGTATTGAGAGCTCC
TCCTTTGAATCTGTGAGGAACAGATGGTCTTTTTTCACGAGATACATAAACAACTAGT
GGCATTTCTTTTTGGTCATTAATGATCTCGATCCGAGAAGGTCTATCGGTCACCATAC
AAAGATTCTTTAGGTTGCTTCCGAATTTCTCAATATTTTTCTGCATTTTCTCGTATTTGG
CTTTGGTGGGCATATGTTCCTTGGAGTTTTCAACTTGTGATGAGTTAGGATCCACTCTA
TTTAATCTCTTCTTAATCTGGTCCCTCACTTCTTCAAATTCACGAGGTCGATGAAGTCG
TTCATTCTCACCCAAAGCAGTGAAGAAAAACTTAGGACACCTTGACTTAACATCATAT
TTTTTACAAAAAGGAACCCAAACTTTAGCAAATTCAAAAGCCTCTTTGATCCCAAAAA
GAGTAATAGGAGAACCTCCATCATCAGAAAGATAAATAGAAAGTTTATTAGAAGGGT
AATCCATTGCAATAGCAGAAATAACAGTGTTCATAACATCAACCGTTGGTTCTTTTTC
AGGATCAATGGTACACACAAATATGTCGAGTCCCGGCAACTTCTCCTCCGGCGGTAA
TTTCTCGGTCATAACTGAACGATTCACCAGCCTCCAACGGAATGCTTGGTTGAAAAAC
CATAGAAATGATAGAATAATCTCAGCTATTGTCATTAGAAACCATGGATATGAAATA
AACAAATTGCTGATACGGTAGTAAAAGAGAAACAAGACACATGTGAAGTGGAAGAT
TATGTAAGCTCTTCTTAGAGGTAACAATGGTTGAACTGTTTCTTTGTGAAATGTGAAG
GTTGCCAT

FIGS. 46A-46E show that the formation of glycyrrhetinic acid 3-O-monoglucuronide is catalyzed by GuCSLG. (FIG. 46A) EIC of glycyrrhetinic acid 3-O-monoglucuronide [m/z=645.36, in negative ion mode] of samples from N. benthamiana expressing proteins from Glycyrrhiza uralensis: bAS—β-amyrin synthase (SEQ ID NO: 48); CYP88D6—β-amyrin 11-oxidase (SEQ ID NO: 77); CYP72A154—11-oxo-b-amyrin 30-oxidase (SEQ ID NO: 79); GuUGAT (SEQ ID NO: 83)-glycosyltransferase previously reported to perform glucuronation of glycyrrhetinic acid; UGT73P12 (SEQ ID NO: 85)—glycosyltransferase transferring glcA onto glycyrrhetinic acid 3-O-monoglucuronide; GuCSL—cellulose synthase like G. (FIG. 46B) MS/MS of 654.36 (glycyrrhetinic acid 3-O-monoglucuronide isomer 1; 55 V). (FIG. 46C) MS/MS of 654.36 (isomer 2; 55 V). (FIG. 46D) Structure of the glycyrrhetinic acid 3-O-monoglucuronide with fragmentation patterns indicated by arrows. (FIG. 46E) Nomenclature of the cross-ring fragmentation of glucuronic acid with detected ions.

FIGS. 47A-47F show formation of glycyrrhizin (glycyrrhetinic acid 3-O-diglucuronide) in N. benthamiana requires GuCSLG. (FIG. 47A) EIC of glycyrrhizin [m/z=821.40, in negative ion mode] of samples from N. benthamiana expressing proteins from G. uralensis. bAS—b-amyrin synthase [β-amyrin synthase] (SEQ ID NO: 48); CYP88D6—b-amyrin 11-oxidase (SEQ ID NO: 76 [gene] and SEQ ID NO: 77 [polypeptide]); CYP72A154—11-oxo-b-amyrin 47-oxidase (SEQ ID NO: 78 [gene] and SEQ ID NO: 79 [polypeptide]); GuUGAT (SEQ ID NO: 82 [gene] and SEQ ID NO: 83 [polypeptide])—glycosyltransferase previously reported to perform glucuronation of glycyrrhetinic acid; UGT73P12—glycosyltransferase transferring glcA onto glycyrrhetinic acid 3-O-monoglucuronide (SEQ ID NO: 84 [gene] and SEQ ID NO: 85 [polypeptide]); GuCSL—cellulose synthase like G (SEQ ID NO: 80 or SEQ ID NO: 103 [gene], and SEQ ID NO: 81 or SEQ ID NO: 102 [polypeptide]). (FIG. 47B) MS/MS (65 V) of 821.40 (isomer 1)—product of bAS (SEQ ID NO: 45)+CYP88D6 (SEQ ID NO: 77)+CYP72A154 (SEQ ID NO: 79)+GuCSL (SEQ ID NO: 81 or SEQ ID NO: 102)+UGT73P12 (SEQ ID NO: 85). (FIG. 47C) MS/MS (65 V) of 821.40 (isomer 2)—product of bAS (SEQ ID NO: 45)+CYP88D6 (SEQ ID NO: 77)+CYP72A154 (SEQ ID NO: 79)+GuCSL (SEQ ID NO: 81 or SEQ ID NO: 102)+UGT73P12 (SEQ ID NO: 85). (FIG. 47D) and (FIG. 47E) MS/MS of glycyrrhizin authentic standard, isomer 1 and 2 respectively. (FIG. 47F) Structure of glycyrrhizin with fragmentation patterns indicated by arrows.

FIG. 48 presents the structure of SOAP5. (FIG. 48) Alignment of protein sequences of Arabidopsis AtCESA1 (SEQ ID NO: 67), AtCESA3 (SEQ ID NO: 68), and their orthologs in spinach (SoCESA1 (SEQ ID NO: 70) and SoCESA3 (SEQ ID NO: 71)) together with SOAP5 (SEQ ID NO: 66) and its counterpart in Arabidopsis—AtCSLG1 (SEQ ID NO: 69).

FIGS. 49A-49C present the predicted 3D structure of SOAP5. (FIGS. 49A and 49B) 3D model of SOAP5 with predicted tunnels, allowing medicagenic acid to reach the active site of the enzyme (blue—1,2,3) and to leave (yellow—4) the hydrophobic environment of the lipid bilayer. (FIG. 49C) Profiles and properties of the tunnels shown in (FIG. 49A).

FIGS. 50A and 50B present site directed mutagenesis data of SOAP5. (FIG. 50A) EICs of medicagenic acid [m/z=501.32] and medicagenic acid 3-GlcA [m/z=677.35] from plants transiently expressing SOAP1-4 with mutated versions of SOAP5 compared to native SOAP5, SOAP1-4 alone and control (EV). (FIG. 50B) Accumulation of MA and MA-3-GlcA in plants expressing mutated variants of SOAP5 represented as a ratio of MA-3-GlcA to free MA. The ordinate axis is in log₁₀ scale. Values represent mean of three independent biological experiments. Statistically significant differences compared with plants expressing native SOAP5 are indicated; ***P<0.001.

FIGS. 51A-51H present micrographs showing that SOAP proteins including SOAP5 are co-localized in the endoplasmic reticulum (ER) membrane. (FIGS. 51A-51F) Confocal images of SOAP5:RFP transiently co-expressed in N. benthamiana leaves with (FIG. 51A) ER:GFP marker; (FIG. 51B) Golgi:GFP marker; (FIG. 51C) SOAP1:GFP; (FIG. 51D) SOAP2:GFP; (FIG. 51E) SOAP3:GFP and SOAP4:GFP (FIG. 51F). Each panel contains images of GFP, RFP and both channels merged. Scale bar—50 μm. (FIG. 51G) Topology model of SOAP5 with conserved motifs (red), substituted amino acids (blue, pointed with arrows) and six transmembrane domains indicated. (FIG. 51H) FRET index of cells that expressed the indicated constructs. The values are means±SE. Significant differences comparing to negative control expressing free GFP and RFP (NC) are indicated; *P<0.05; **P<0.01.

FIGS. 52A-52F present micrographs showing SOAPs involved in triterpenoid aglycon formation are colocalized and reside in endoplasmic reticulum (ER). (FIGS. 52A-52F) Confocal images of SOAPs in fusion with fluorescent proteins (GFP and RFP) transiently co-expressed in N. benthamiana leaves. (FIG. 52A) SOAP1:GFP+SOAP2:RFP; (FIG. 52B) SOAP1:GFP+SOAP3:RFP; (FIG. 52C) SOAP1:GFP+SOAP4:RFP; (FIG. 52D) SOAP2:GFP+SOAP3:RFP; (FIG. 52E) SOAP2-GFP+SOAP4:RFP; (FIG. 35F) SOAP3:GFP+SOAP4:RFP (F). Each panel contains images of GFP, RFP and both channels merged together. Scale bar—50 picometers.

FIG. 53 present data showing that SOAPs in fusion with fluorescent proteins (FP) are still active. EICs of medicagenic acid [m/z=501.32] and medicagenic acid 3-GlcA [m/z=677.35] from N. benthamiana plants transiently expressing SOAP1:eGFP, SOAP2:eGFP, SOAP3:mRFP, SOAP4:eGFP and SOAP5:mRFP compared to native SOAP5, SOAP1-4 alone and control (EV). SOAP1:GFP, SOAP2:GFP, SOAP3:RFP, SOAP4:GFP and SOAP5:RFP compared to native SOAP1-5, SOAP1-4 alone and control (EV).

FIGS. 54A-54B present SOAPs in fusion with fluorescent proteins (FP) are still active. (FIG. 54A) Fluorescence resonance energy transfer (FRET) index of cells that expressed the indicated constructs. The values are means±SE. Significant differences comparing to negative control expressing free GFP and RFP (NC) are indicated with asterisks; *P<0.05; **P<0.01. (FIG. 54B) Schematic representation of possible protein localization within ER.

FIG. 55 depicts a gene editing design of Yellow Pea GAME 15 (CSLG). The schematic shows the Yellow Pea GAME15 gene and the Exon locations and the locations of the three guide RNAs used. For ease of viewing, the figure is cut in half 3′ of Exon 4 and Exon 4 is again included in the lower portion to aid in alignment.

FIG. 56 presents a T-DNA vector including CAS9 and guide RNAs against Yellow Pea GAME 15 (CSLG). For ease of viewing, the figure is cut in the middle of the CAS9 gene, wherein an overlapping portion of the CAS9 gene is included in the lower portion to aid in alignment.

FIG. 57 presents the results of DNA amplification of target gene-edited sequence.

FIG. 58 presents sequencing result of editing target sequence (1^{st} generation). The presentation is cut in half 3′ of the TaqII site, which portion is duplicated in the lower half as well to aid in alignment.

FIG. 59 presents sequencing result of editing target sequence (2^{nd} generation). The presentation is cut in half 3′ of the TaqII site, which is shown in the lower half as well to aid in alignment.

FIGS. 60A-60B present chemical structures of two major pea saponins, saponin Bb also known as soyasaponin I (Compound 36 in Table 2; FIG. 60A) and saponin βg also known as soyasaponin VI (Compound 35 in Table 2; FIG. 60B). DDMP—2,3-dihydro-2,5-dihydroxy-6-methyl-4H-pyran-4-one.

FIGS. 61A-61D shows identification of the two major pea saponins in non-edited wild type plant. FIGS. 61A-61B present the identification of saponin βg (soyasaponin VI) and FIGS. 61C-61D present the identification of saponin Bb (soyasaponin I).

FIG. 62 shows reduction of saponin Bb and saponin βg in homozygous pea mutant vs non-edited wild type plant.

### DETAILED DESCRIPTION

In the following detailed description, numerous specific details are set forth in order to provide a thorough understanding of the invention. However, it will be understood by those skilled in the art that the present invention may be practiced without these specific details. In other instances, well-known methods, procedures, and components have not been described in detail so as not to obscure the present invention.

Specifically disclosed herein are genetically modified cells having decreased expression of at least one heterologous gene compared to a corresponding unmodified cell, said at least one heterologous gene encoding a cellulose synthase like G (CSLG) enzyme, wherein said genetically modified cell comprises a decreased content of at least one steroidal alkaloid, a derivative thereof, a metabolite thereof, or a biosynthetic intermediate thereof, of at least one steroidal saponin, a derivative thereof, a metabolite thereof, or a biosynthetic intermediate thereof, or of at least one triterpenoid saponin, a derivative thereof, a metabolite thereof, or a biosynthetic intermediate thereof, compared to the corresponding unmodified cell. Specifically disclosed herein are genetically modified cells having decreased expression of at least one heterologous gene compared to a corresponding unmodified cell, said at least one heterologous gene encoding a cellulose synthase like G (CSLG) enzyme, wherein said genetically modified cell comprises an increased content of at least one steroidal alkaloid, a derivative thereof, a metabolite thereof, or a biosynthetic intermediate thereof, of at least one steroidal saponin, a derivative thereof, a metabolite thereof, or a biosynthetic intermediate thereof, or of at least one triterpenoid saponin, a derivative thereof, a metabolite thereof, or a biosynthetic intermediate thereof, compared to the corresponding unmodified cell. Specifically disclosed herein are genetically modified cells having decreased expression of at least one heterologous gene compared to a corresponding unmodified cell, said at least one heterologous gene encoding a cellulose synthase like G (CSLG) enzyme, wherein said genetically modified cell comprises an increased, decreased or a combination thereof, content of at least one steroidal alkaloid, a derivative thereof, a metabolite thereof, or a biosynthetic intermediate thereof, of at least one steroidal saponin, a derivative thereof, a metabolite thereof, or a biosynthetic intermediate thereof, or of at least one triterpenoid saponin, a derivative thereof, a metabolite thereof, or a biosynthetic intermediate thereof, compared to the corresponding unmodified cell. In some embodiments, the genetically modified cell expressing a heterologous CSLG further expresses at least one additional heterologous gene encoding an enzyme, said enzyme selected from the group consisting of a saponin beta-amyrin synthase, a cytochrome P450, a glycosyltransferase, an acyltransferase, and an UDP-glucose 6-dehydrogenase 1, or any combination thereof.

The term “CSLG” encompasses Cellulose Synthase Like enzymes from the G family. There are various cellulose synthase Like (CSL) families, each with a different suffix, for example, A, B, E, and G. The suffix for each family is assigned according to enzyme's similarity to previously characterized proteins from Arabidopsis thaliana. CSL enzymes from different families have different technical characteristics. As used throughout, the terms “CSL” without a suffix of A, B, or E, in certain embodiments, may be used interchangeably with the term “CSLG”, having all the same meaning and qualities.

As used herein in some embodiments, the terms “CSLG”, “GAME15”, “SOAP5”, and “CSyL”, may be used interchangeably having all the same meanings and qualities.

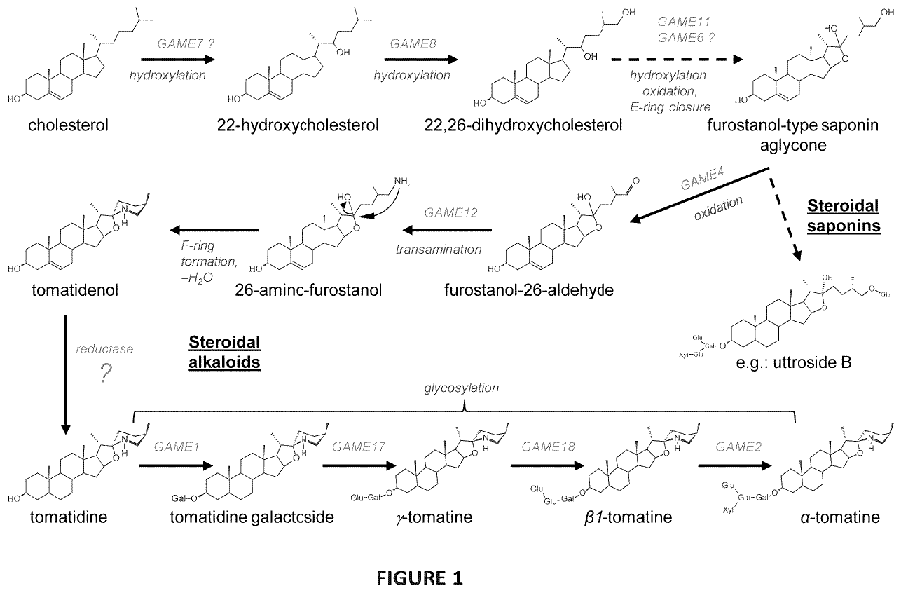

Described herein are at least seven tested CSLG genes, which in certain embodiments include the GAME15 genes and their encoded enzymes (6 enzymes from 6 species—tomato, wild tomato, potato, wild potato, eggplant, and pepper), which transfer Glucoronic Acid or other sugar moieties to generate steroidal alkaloids and which transfer Glucoronic Acid or other sugar moieties on furostanol-type saponin (aglycone) to generate steroidal saponins or precursors thereof or to generate steroidal alkaloids or precursors thereof (see, e.g., FIG. 1).

Described herein are seven tested CSLG genes (also known as SOAP5 genes) and their encoded enzymes (7 enzymes from 7 species—spinach, Chinese licorice, red beet, quinoa, alfalfa, soybean, and Lotus japonicum), which transfer glucuronic acid on triterpenoid aglycone. The term “triterpenoid aglycone” may in some embodiments, encompass the group of compounds derived from beta-amyrin. Thus, CSLG enzymes disclosed herein provide a unique activity.

A skilled artisan would appreciate that altering the content of at least one steroidal alkaloid, steroidal saponin, or triterpenoid saponin in a cell, for example a plant cell, a yeast cell, an algal cell, a bacterial cell, may comprise mutating an endogenous CSLG gene or regulating the CSLG gene expression, or may comprise adding a heterologous CSLG to a cell that does not express a CSLG enzyme or in order to supplement the activity of a CSLG enzyme expressed in the heterologous cell. In some embodiments, the plant cell may be comprised within a plant or plant part.

According to certain embodiments, expression of the at least one gene or any combination thereof is altered, the altering comprising mutagenizing the at least one gene, wherein the mutagenesis comprises introduction of one or more point mutations, or genome editing, or use or a bacterial CRISPR/CAS system, or a combination thereof. In certain embodiments, expression of the CSLG gene or polynucleotide is silenced, repressed, or reduced, e.g., by deletion, insertion or modification, or by introduction of at least one silencing molecule targeted to a CSLG gene.

It is to be understood that increasing the expression of the at least one gene or combination thereof may be achieved by various means, all of which are explicitly encompassed within the scope of present invention. According to certain embodiments, enhancing the expression of CSLG can be affected at the genomic and/or the transcript level using a variety of molecules that enhance transcription and/or translation including, but not limited to, transcription factors. Inserting a mutation to the at least one gene, including addition of promoters, enhancers, and the like can be also used, as long as the mutation results in up-regulation of the gene expression. According to other embodiments, expression is increased at the protein level using agonists and the like.

In some embodiments, these genetically modified cells produce steroidal alkaloids. In some embodiments, these genetically modified cells produce steroidal saponins. In some embodiments, these genetically modified cells produce triterpenoid saponins. In some embodiments, the genetically modified cells comprise an increased content of at least a steroidal alkaloid, a derivative thereof, a metabolite thereof, or a biosynthetic intermediate thereof, compared to a corresponding unmodified cell. In some embodiments, the genetically modified cells comprise an increased content of at least a steroidal saponin, a derivative thereof, a metabolite thereof, or a biosynthetic intermediate thereof, compared to a corresponding unmodified cell. In some embodiments, the genetically modified cells comprise an increased content of at least a triterpenoid saponin, a derivative thereof, a metabolite thereof, or a biosynthetic intermediate thereof, compared to a corresponding unmodified cell.

In some embodiments, derivatives of steroidal alkaloids, steroidal saponins, or triterpenoid saponins comprise glycosylated derivatives of, respectively, steroidal alkaloids, steroidal saponins, or triterpenoid saponins.

In some embodiments, the nucleic acid sequence encoding said at least one heterologous CSLG gene is set forth in any one of SEQ ID NOS: 125, 127, 30, 32, 34, 36, 38, 40, 65, 80, 93, 95, 97, 99, 101, 103, or 105; or a homolog thereof having at least 55% identity to and at least 55% coverage of, the nucleic acid sequence set forth in any one of SEQ ID NOS: 125, 127, 30, 32, 34, 36, 38, 40, 65, 80, 93, 95, 97, 99, 101, 103, or 105. In some embodiments, the nucleic acid sequence encoding said at least one heterologous CSLG gene is set forth in any one of SEQ ID NOS: 125, 127, 30, 32, 34, 36, 38, 40, 65, 80, 93, 95, 97, 99, 101, 103, or 105; or a homolog thereof having at least 55% identity to and at least 80% coverage of, the nucleic acid sequence set forth in any one of SEQ ID NOS: 125, 127, 30, 32, 34, 36, 38, 40, 65, 80, 93, 95, 97, 99, 101, 103, or 105.

In some embodiments, a CSLG enzyme homolog has at least 55%, 60%, 65%, 70%, 75%, 80%, 85%, 90%, 95% 98%, or 99% identity of the amino acid sequence. In some embodiments, a gene encoding a CSLG enzyme homolog has at least 55%, 60%, 65%, 70%, 75%, 80%, 85%, 90%, 95% 98%, or 99% identity of the nucleic acid sequence.

In some embodiments, a CSLG enzyme homolog has at least 55%, 60%, 65%, 70%, 75%, 80%, 85%, 90%, 95% 98%, 99%, or 100% coverage of the amino acid sequence. In some embodiments, a gene encoding a CSLG enzyme homolog has at least 55%, 60%, 65%, 70%, 75%, 80%, 85%, 90%, 95% 98%, 99%, or 100% coverage of the nucleic acid sequence.

In some embodiments, the nucleic acid sequence encoding said at least one heterologous CSLG gene is set forth in any one of SEQ ID NOS: 125, 127, 30, 32, 34, 36, 38, 40, 65, 80, 93, 95, 97, 99, 101, 103, or 105; or a homolog thereof having at least 80% identity to and at least 80% coverage of, the nucleic acid sequence set forth in any one of SEQ ID NOS: 125, 127, 30, 32, 34, 36, 38, 40, 65, 80, 93, 95, 97, 99, 101, 103, or 105.

In some embodiments, the amino acid sequence of said encoded CSLG enzyme is set forth in any one of SEQ ID NOS: 126, 31, 33, 35, 37, 39, 41, 66, 81, 94, 96, 98, 100, 102, or 104; or a homolog thereof having at least 55% identity to and at least 55% coverage of, the amino acid sequence set forth in any one of SEQ ID NOS: 126, 31, 33, 35, 37, 39, 41, 66, 81, 94, 96, 98, 100, 102, or 104. In some embodiments, the amino acid sequence of said encoded CSLG enzyme is set forth in any one of SEQ ID NOS: 126, 31, 33, 35, 37, 39, 41, 66, 81, 94, 96, 98, 100, 102, or 104; or a homolog thereof having at least 55% identity to and at least 80% coverage of, the amino acid sequence set forth in any one of SEQ ID NOS: 126, 31, 33, 35, 37, 39, 41, 66, 81, 94, 96, 98, 100, 102, or 104.

In some embodiments, the amino acid sequence of said encoded CSLG enzyme is set forth in any one of SEQ ID NOS: 126, 31, 33, 35, 37, 39, 41, 66, 81, 94, 96, 98, 100, 102, or 104; or a homolog thereof having at least 80% identity to and at least 80% coverage of, the amino acid sequence set forth in any one of SEQ ID NOS: 126, 31, 33, 35, 37, 39, 41, 66, 81, 94, 96, 98, 100, 102, or 104.

In some embodiments, the nucleic acid sequence encoding said at least one additional heterologous gene encodes: (a) a β-amyrin synthase, said nucleic acid sequence set forth in SEQ ID NO: 45; or a homolog thereof having at least 80% identity to and at least 80% coverage of, the nucleic acid sequence set forth in SEQ ID NO: 45; or (b) a cytochrome P450, said nucleic acid sequence set forth in any one of SEQ ID NO: 46, 51, or 53; or a homolog thereof having at least 80% identity to and at least 80% coverage of, the nucleic acid sequence set forth in any one of SEQ ID NO: 46, 51, or 53; or a (c) glycosyl transferase, said nucleic acid sequence set forth in any one of SEQ ID NO: 55, 57, 59, or 61; or a homolog thereof having at least 80% identity to and at least 80% coverage of, the nucleic acid sequence set forth in any one of SEQ ID NO: 55, 57, 59, or 61; or (d) an acetyltransferase, said nucleic acid sequence set forth in SEQ ID NO: 63; or a homolog thereof having at least 80% identity to and at least 80% coverage of, the nucleic acid sequence set forth in SEQ ID NO: 63; or (e) a UDP-glucose 6-dehydrogenase 1, said nucleic acid sequence set forth in SEQ ID NO: 74; or a homolog thereof having at least 80% identity to and at least 80% coverage of, the nucleic acid sequence set forth in SEQ ID NO: 74; or (f) any combination thereof of (a), (b), (c), (d), and (e).

In some embodiments, the amino acid sequence of said encoded at least one additional heterologous gene encodes: (a) a β-amyrin synthase, said amino acid sequence set forth in SEQ ID NO: 48; or a homolog thereof having at least 80% identity to and at least 80% coverage of, the amino acid sequence set forth in SEQ ID NO: 48; or (b) a cytochrome P450, said amino acid sequence set forth in any one of SEQ ID NO: 49, 52, or 54; or a homolog thereof having at least 80% identity to and at least 80% coverage of, the amino acid sequence set forth in any one of SEQ ID NO: 49, 52, or 54; or a (c) glycosyl transferase, said amino acid sequence set forth in any one of SEQ ID NO: 56, 58, 60, or 62; or a homolog thereof having at least 80% identity to and at least 80% coverage of, the amino acid sequence set forth in any one of SEQ ID NO: 56, 58, 60, or 62; or (d) an acetyltransferase, said amino acid sequence set forth in SEQ ID NO: 64; or a homolog thereof having at least 80% identity to and at least 80% coverage of, the amino acid sequence set forth in SEQ ID NO: 64; or (e) a UDP-glucose 6-dehydrogenase 1, said amino acid sequence set forth in SEQ ID NO: 75; or a homolog thereof having at least 80% identity to and at least 80% coverage of, the amino acid sequence set forth in SEQ ID NO: 75; or (f) any combination thereof of (a), (b), (c), (d), and (e).

Heterologous expression encompasses the expression of a gene or part of a gene in a host cell or host organism, for example but not limited to a plant cell, a yeast cell, an alga, or a plant, which does not naturally have this gene or gene fragment. Insertion of the gene in the heterologous host may be performed by any recombinant DNA technology known in the art, or for example in the case of yeast, may be performed by mating.

The triterpenoid saponin or said derivative, metabolite, or biosynthetic intermediate thereof, may have a broad range of commercial uses and may in some embodiments comprise a sweetener, foaming agent, emulsifier, preservative, anti-carcinogen, hypocholesterolemic agent, anti-inflammatory agent, anti-oxidant, biological adjuvant, anti-microbial agent, insecticidal agent, antifeedant, anti-fungal agent, or any combination thereof.

In some embodiments, (a) said steroidal glycoalkaloid comprises alpha-tomatine, tomatine, dehydrotomatine, hydroxytomatine, acetoxytomatine, dihydroxytomatine, alpha-chaconine, alpha-solanine, alpha-solasonine, alpha-solmargine, and their derivatives, related compounds or any combination thereof; (b) said steroidal saponin comprises uttroside B, tomatosides and their derivatives, related compounds, or any combination thereof, (c) said triterpenoid saponin comprises medicagenic acid 3-O-glucuronide (MA-3-GlcA) (Compound 6), Compound 7, Compound 8, Compound 9, Compound 10, Compound 11, glycyrrhizin (Compound 14), glycyrrhetinic acid 3-O-monoglucuronide (Compound 15), bayogenin (Compound 25), bayogenin-hexA-hex-hex (Compound 31), serjanic acid (Compound 26), serjanic acid-hexA-hex (Compound 32), soyasapogenol A (Compound 29), soyasapogenol B (Compound 30), soyasapogenol A-hexA-hex-pent (Compound 34), soyasaponin VI (Compound 35), soyasaponin I, betavulgaroside IV (Compound 33), hederagenin-3GlcA, gypsogenin-3GlcA, gypsogenic acid-3GlcA, or a QS-21 adjuvant, or any combination thereof; (d) the biosynthetic intermediate of said triterpenoid saponin comprises Compound 1, Compound 2, medicagenic acid (Compound 5), oleanolic acid (Compound 3), oleanolic acid-3GlcA, augustic acid (Compound 4), augustic acid-3GlcA, or glycyrrhetinic acid (Compound 13), or any combination thereof, or (e) any combination of these.

According to certain exemplary embodiments, the downstream steroidal glycoalkaloid is selected from the group consisting of esculeosides or dehydroesculeosides.

In some embodiments, the genetically modified cell further comprises an altered content of a phytosterol, or a derivative thereof, a metabolite thereof, or a biosynthetic intermediate thereof, a cholesterol, or a derivative thereof, a metabolite thereof, or a biosynthetic intermediate thereof, a phytocholesterol, or a derivative thereof, a metabolite thereof, or a biosynthetic intermediate thereof; a cholestenol, or a derivative thereof, a metabolite thereof, or a biosynthetic intermediate thereof, or a phytocholestenol, or a derivative thereof, a metabolite thereof, or a biosynthetic intermediate thereof.

In some embodiments, an “altered content” encompasses an increased content of a phytosterol/a phytocholesterol/phytocholestenol, derivative thereof, metabolike thereof, or biosynthetic intermediate thereof. In some embodiments, an “altered content” encompasses an increased content of a phytosterol, derivative thereof, metabolike thereof, or biosynthetic intermediate thereof.

In some embodiments, the genetically modified cell further comprises a reduced content of a phytosterol, or a derivative thereof, a metabolite thereof, or a biosynthetic intermediate thereof, a cholesterol, or a derivative thereof, a metabolite thereof, or a biosynthetic intermediate thereof, a phytocholesterol, or a derivative thereof, a metabolite thereof, or a biosynthetic intermediate thereof, a cholestenol, or a derivative thereof, a metabolite thereof, or a biosynthetic intermediate thereof, or a phytocholesterol, or a derivative thereof, a metabolite thereof, or a biosynthetic intermediate thereof.

In some embodiments, the genetically modified cells are plant cells, yeast cells, or alga cells. In some embodiments, the genetically modified plant cells are comprised within a plant or a plant part. In some embodiments, the genetically modified cells are yeast cells. In some embodiments, the genetically modified cells are alga cells.

In some embodiments, said plant cell comprises a cell from a plant in the Poales order, the Caryophyllales order, the Solanales order, the Fabales order, the Malvales order, the Apiales order, the Brassicales order, the Asparagales order, the Dioscoreales order, or the Liliales order.

In some embodiments, (a) when said plant cell comprises a cell from a plant in the Poales order, said plant is selected from the group of genera consisting of the Oryza genus, the Hordeum genus, the Avena genus, and the Triticum genus; (b) when said plant cell comprises a cell from a plant in the Caryophyllales order, said plant is selected from the group of genera consisting of the Spinacia genus, the Chenopodium genus, the Beta genus, the Rheum genus, the Vaccaria genus, the Saponaria genus, and the Gypsophila genus; (c) when said plant cell comprises a cell from a plant in the Solanales order, said plant is selected from the group of genera consisting of the Solanum genus, the Capsicum genus, the Nicotiana genus, the Hyoscyamus genus, the Datura genus, and the Atropa genus; (d) when said plant cell comprises a cell from a plant in the Fabales order, said plant is selected from the group of genera consisting of the Glycyrrhiza genus, the Medicago genus, the Glycine genus, the Lotus genus, the Cicer genus, the Phaseolus genus, the Pisum genus, the Arachis genus, the Lupinus genus, and the Acacia genus; (e) when said plant cell comprises a cell from a plant in the Malvales order, said plant is selected from the Theobroma genus; (f) when said plant cell comprises a cell from a plant in the Apiales order, said plant is selected from the group of genera consisting of the Daucus genus, the Apium genus, the Petroselinum genus, the Panax genus, the Bupleurum genus, the Hedera genus, and the Centella genus; or (g) when said plant cell comprises a cell from a plant in the Brassicales order, said plant is selected from the group of genera consisting of the Arabidopsis genus, the Brassica genus, the Capparis genus, and the Carica genus.

In some embodiments, (a) when said plant cell comprises a cell from a plant in the Caryophyllales order, said plant is selected from the group consisting of spinach, beetroot, and quinoa; (b) when said plant cell comprises a cell from a plant in the Solanales order, said plant is selected from the group consisting of tomato, wild tomato, potato, wild potato, eggplant, pepper, bell pepper, cayenne pepper, chili pepper, pimiento, tabasco pepper, ground cherry, tobacco, and bittersweet; or (c) when said plant cell comprises a cell from a plant in the Fabales order, said plant is selected from the group consisting of pea, alfalfa, soy, Lotus japonicus, and licorice.

In some embodiments, the plant cell is from a tomato plant having: (i) an increased content of alpha-tomatine or a derivative thereof, a metabolite thereof, or a biosynthetic intermediate thereof, or tomatidine or a derivative thereof, a metabolite thereof, or a biosynthetic intermediate thereof, or a steroidal saponin or a derivative thereof, a metabolite thereof, or a biosynthetic intermediate thereof, or (ii) an altered content of a phytosterol or a derivative thereof, a metabolite thereof, or a biosynthetic intermediate thereof; a cholesterol or a derivative thereof, a metabolite thereof, or a biosynthetic intermediate thereof, a phytocholesterol or a derivative thereof, a metabolite thereof, or a biosynthetic intermediate thereof, a cholestenol or a derivative thereof, a metabolite thereof, or a biosynthetic intermediate thereof, or a phytocholestenol or a derivative thereof, a metabolite thereof, or a biosynthetic intermediate thereof.

In some embodiments, the plant cell is from a tomato plant having: (i) an increased content of alpha-tomatine or a derivative thereof, a metabolite thereof, or a biosynthetic intermediate thereof, or tomatidine or a derivative thereof, a metabolite thereof, or a biosynthetic intermediate thereof, or a steroidal saponin or a derivative thereof, a metabolite thereof, or a biosynthetic intermediate thereof; or (ii) a reduced content of a phytosterol or a derivative thereof, a metabolite thereof, or a biosynthetic intermediate thereof; a cholesterol or a derivative thereof, a metabolite thereof, or a biosynthetic intermediate thereof, a phytocholesterol or a derivative thereof, a metabolite thereof, or a biosynthetic intermediate thereof, a cholestenol or a derivative thereof, a metabolite thereof, or a biosynthetic intermediate thereof, or a phytocholesterol or a derivative thereof, a metabolite thereof, or a biosynthetic intermediate thereof.

In some embodiments, the plant cell is from a potato plant having: (i) an increased content of alpha-chaconine or a derivative thereof, a metabolite thereof, or a biosynthetic intermediate thereof, or alpha-solanine or a derivative thereof, a metabolite thereof, or a biosynthetic intermediate thereof, or a steroidal saponin or a derivative thereof, a metabolite thereof, or a biosynthetic intermediate thereof, or (ii) an altered content of a phytosterol or a derivative thereof, a metabolite thereof, or a biosynthetic intermediate thereof, a cholesterol or a derivative thereof, a metabolite thereof, or a biosynthetic intermediate thereof, a phytocholesterol or a derivative thereof, a metabolite thereof, or a biosynthetic intermediate thereof, a cholestenol or a derivative thereof, a metabolite thereof, or a biosynthetic intermediate thereof, or a phytocholestenol or a derivative thereof, a metabolite thereof, or a biosynthetic intermediate thereof.

In some embodiments, the plant cell is from a potato plant having: (i) an increased content of alpha-chaconine or a derivative thereof, a metabolite thereof, or a biosynthetic intermediate thereof, or alpha-solanine or a derivative thereof, a metabolite thereof, or a biosynthetic intermediate thereof, or a steroidal saponin or a derivative thereof, a metabolite thereof, or a biosynthetic intermediate thereof, or (ii) a reduced content of a phytosterol or a derivative thereof, a metabolite thereof, or a biosynthetic intermediate thereof, a cholesterol or a derivative thereof, a metabolite thereof, or a biosynthetic intermediate thereof, a phytocholesterol or a derivative thereof, a metabolite thereof, or a biosynthetic intermediate thereof, a cholestenol or a derivative thereof, a metabolite thereof, or a biosynthetic intermediate thereof, or a phytocholestenol or a derivative thereof, a metabolite thereof, or a biosynthetic intermediate thereof.

In some embodiments, the plant cell is from an eggplant plant having: (i) an increased content of alpha-solasonine or a derivative thereof, a metabolite thereof, or a biosynthetic intermediate thereof, or alpha-solamargine or a derivative thereof, a metabolite thereof, or a biosynthetic intermediate thereof, or a steroidal saponin or a derivative thereof, a metabolite thereof, or a biosynthetic intermediate thereof, or (ii) an altered content of a phytosterol or a derivative thereof, a metabolite thereof, or a biosynthetic intermediate thereof, a cholesterol or a derivative thereof, a metabolite thereof, or a biosynthetic intermediate thereof, a phytocholesterol or a derivative thereof, a metabolite thereof, or a biosynthetic intermediate thereof, a cholestenol or a derivative thereof, a metabolite thereof, or a biosynthetic intermediate thereof, or a phytocholestenol or a derivative thereof, a metabolite thereof, or a biosynthetic intermediate thereof.

In some embodiments, the plant cell is from an eggplant plant having: (i) an increased content of alpha-solasonine or a derivative thereof, a metabolite thereof, or a biosynthetic intermediate thereof, or alpha-solamargine or a derivative thereof, a metabolite thereof, or a biosynthetic intermediate thereof, or a steroidal saponin or a derivative thereof, a metabolite thereof, or a biosynthetic intermediate thereof, or (ii) a reduced content of a phytosterol or a derivative thereof, a metabolite thereof, or a biosynthetic intermediate thereof, a cholesterol or a derivative thereof, a metabolite thereof, or a biosynthetic intermediate thereof, a phytocholesterol or a derivative thereof, a metabolite thereof, or a biosynthetic intermediate thereof, a cholestenol or a derivative thereof, a metabolite thereof, or a biosynthetic intermediate thereof, or a phytocholestenol or a derivative thereof, a metabolite thereof, or a biosynthetic intermediate thereof.

In some embodiments, the plant cell is from a pea plant having a reduced content of of soyasaponin I or soyasaponin VI or a combination thereof.

In some embodiments, said plant cell comprises a leaf cell, a petiole cell, a plant stem or stalk cell, a root cell, a bud cell, a tuber cell, a bean cell, a grain or kernel cell, a fruit cell, a nut cell, a legume cell, a seed or seed cell, a callus cell, a bract cell, or a flower cell. In some embodiments, said plant cell is comprised in a plant or a portion thereof, said portion thereof comprising a plant leaf, a plant petiole, a plant stem or stalk, a plant root, a plant bud, a plant tuber, a plant bean, a plant grain or kernel, a plant fruit, a plant nut, a plant legume, a plant seed, a plant bract, or a plant flower.

In some embodiments, said yeast is selected from a Saccharomyces genus, a Schizosaccharomyces genus, a Pichia genus, a Yarrowia genus, a Kluyveromyces genus, or a Candida genus.

In some embodiments, said alga is selected from a microalga, a multi-cellular alga, a cyanobacterium, a diatom, chlorophytes (green algae), rhodophytes (red algae), or phaeo-phytes (brown algae), a Dunaliella, a Chlamydamonas, or a Hematococus.

Specifically disclosed herein are genetically modified plants comprising at least one cell having altered expression of at least a cellulose synthase like G (CSLG) gene compared to the expression of CSLG in a corresponding unmodified plant, and wherein the genetically modified plant has an altered content of at least one steroidal alkaloid, a derivative thereof a metabolite thereof, of at least one steroidal saponin, a derivative thereof, a metabolite thereof, or a biosynthetic intermediate thereof, or of at least one triterpenoid saponin, a derivative thereof, a metabolite thereof, or a biosynthetic intermediate thereof compared to the corresponding unmodified plant.

According to certain embodiments, expression of the at least one gene or any combination thereof is altered, the altering comprising mutagenizing the at least one gene, wherein the mutagenesis comprises introduction of one or more point mutations, or genome editing, or use of a bacterial CRISPR/CAS system, or a combination thereof.

According to certain embodiments, expression of the gene encoding at least one CSLG is elevated compared to its expression in the corresponding unmodified cell or plant, respectively. According to certain embodiments, the genetically modified cell or genetically modified plant comprises a polynucleotide encoding a CSLG, wherein expression of the polynucleotide is selectively increased. According to certain embodiments, the genetically modified plant comprises at least one cell comprising at least one transcribable polynucleotide encoding at least one protein selected from the group consisting of at least one CSLG.

According to certain embodiments, the genetically modified plant is a transgenic plant comprising at least one cell comprising at least one silencing molecule targeted to a polynucleotide encoding at least one CSLG. According to certain embodiments, the transgenic plant comprises a polynucleotide encoding a CSLG, wherein expression of the polynucleotide is selectively silenced, repressed, or reduced. According to certain embodiments, the transgenic plant comprises a polynucleotide encoding a CSLG, wherein the polynucleotide has been selectively edited by deletion, insertion, or modification to silence, repress, or reduce expression thereof, or wherein the genetically modified plant is a progeny of the gene edited plant. According to certain embodiments, the transgenic plant comprises at least one cell comprising at least one silencing molecule targeted to a CSLG gene. According to certain embodiments, the silencing molecule is selected from the group consisting RNA interference molecule and an antisense molecule, or wherein the silencing molecule is a component of a viral induced gene silencing system. According to certain embodiments, the silencing molecule comprises a polynucleotide having a nucleic acid sequence substantially complementary to a region of a CSLG gene.

According to some embodiments, the transgenic plant comprises a plurality of cells comprising the silencing molecule targeted to the at least one CSLG gene. According to additional embodiments, the majority of the plant cells comprise the silencing molecule.

It is to be understood that inhibiting the expression of the at least one gene or combination thereof may be achieved by various means, all of which are explicitly encompassed within the scope of present invention. According to certain embodiments, inhibiting the expression of CSLG can be effected at the genomic and/or the transcript level using a variety of molecules that interfere with transcription and/or translation including, but not limited to, antisense, siRNA, Ribozyme, or DNAzyme molecules. Inserting a mutation to the at least one gene, including deletions, insertions, site specific mutations, zinc-finger nucleases and the like can be also used, as long as the mutation results in down-regulation of the gene expression. According to other embodiments, expression is inhibited at the protein level using antagonists, enzymes that cleave the polypeptide and the like.

In some embodiments, said CSLG gene comprises the endogenous CSLG gene.

In some embodiments, said endogenous CSLG gene comprises a mutation, said mutation comprising at least one or more point mutations, or an insertion, or a deletion, or any combination thereof, and: (a) wherein said expressed CSLG enzyme has increased stability or increased activity or both and the altered content comprises an increased amount of the at least one steroidal alkaloid, a derivative thereof, a metabolite thereof, or a biosynthetic intermediate thereof, of the at least one steroidal saponin, a derivative thereof, a metabolite thereof, or a biosynthetic intermediate thereof, or of the at least one triterpenoid saponin, a derivative thereof, a metabolite thereof, or a biosynthetic intermediate thereof compared to the corresponding unmodified plant; or (b) wherein said expressed CSLG enzyme has decreased stability or decreased activity or both and the altered content comprises a decreased amount of the at least one steroidal alkaloid, a derivative thereof, a metabolite thereof, or a biosynthetic intermediate thereof; of the at least one steroidal saponin, a derivative thereof, a metabolite thereof, or a biosynthetic intermediate thereof, or of the at least one triterpenoid saponin, a derivative thereof, a metabolite thereof, or a biosynthetic intermediate thereof compared to the corresponding unmodified plant.

In some embodiments, (a) said endogenous CSLG gene is selectively silenced, repressed, or has reduced expression and said altered content comprises a reduced amount of the at least one steroidal alkaloid, a derivative thereof, a metabolite thereof, or a biosynthetic intermediate thereof, of the at least one steroidal saponin, a derivative thereof, a metabolite thereof, or a biosynthetic intermediate thereof, or of the at least one triterpenoid saponin, a derivative thereof, a metabolite thereof, or a biosynthetic intermediate thereof compared to the corresponding unmodified plant; or (b) said endogenous CSLG gene is overexpressed and said altered content comprises an increased amount of the at least one steroidal alkaloid, a derivative thereof, a metabolite thereof, or a biosynthetic intermediate thereof, of the at least one steroidal saponin, a derivative thereof, a metabolite thereof, or a biosynthetic intermediate thereof, or of the at least one triterpenoid saponin, a derivative thereof, a metabolite thereof, or a biosynthetic intermediate thereof compared to the corresponding unmodified plant.

In some embodiments, when said endogenous CSLG gene is selectively silenced, repressed, or has reduced expression, said cell further comprises at least one silencing molecule targeted to the polynucleotide encoding said CSLG gene, wherein the silencing molecule is selected from an RNA interference molecule or an antisense molecule, or wherein the silencing molecule is a component of a viral induced gene silencing system.

In some embodiments, said CSLG gene comprises a heterologous CSLG gene, and said altered expression comprises a de novo expression of said gene.

In some embodiments, said expression comprises increased expression compared to a corresponding unmodified cell, and said altered content comprising increased content of at least one steroidal alkaloid, a derivative thereof, a metabolite thereof, or a biosynthetic intermediate thereof, of at least one steroidal saponin, a derivative thereof, a metabolite thereof, or a biosynthetic intermediate thereof, or of at least one triterpenoid saponin, a derivative thereof, a metabolite thereof, or a biosynthetic intermediate thereof, compared to the corresponding unmodified plant.

In some embodiments, the nucleic acid sequence encoding said CSLG gene is set forth in any one of SEQ ID NOS: 125, 127, 30, 32, 34, 36, 38, 40, 65, 80, 93, 95, 97, 99, 101, 103, or 105; or a homolog thereof having at least 55% identity to and at least 55% coverage of, the nucleic acid sequence set forth in any one of SEQ ID NOS: 125, 127, 30, 32, 34, 36, 38, 40, 65, 80, 93, 95, 97, 99, 101, 103, or 105. In some embodiments, the nucleic acid sequence encoding said CSLG gene is set forth in any one of SEQ ID NOS: 125, 127, 30, 32, 34, 36, 38, 40, 65, 80, 93, 95, 97, 99, 101, 103, or 105; or a homolog thereof having at least 55% identity to and at least 80% coverage of, the nucleic acid sequence set forth in any one of SEQ ID NOS: 125, 127, 30, 32, 34, 36, 38, 40, 65, 80, 93, 95, 97, 99, 101, 103, or 105.

In some embodiments, the nucleic acid sequence encoding said CSLG gene is set forth in any one of SEQ ID NOS: 125, 127, 30, 32, 34, 36, 38, 40, 65, 80, 93, 95, 97, 99, 101, 103, or 105; or a homolog thereof having at least 80% identity to and at least 80% coverage of, the nucleic acid sequence set forth in any one of SEQ ID NOS: 125, 127, 30, 32, 34, 36, 38, 40, 65, 80, 93, 95, 97, 99, 101, 103, or 105.

In some embodiments, the amino acid sequence of said CSLG enzyme is set forth in any one of SEQ ID NOS: 126, 31, 33, 35, 37, 39, 41, 66, 81, 94, 96, 98, 100, 102, or 104; or a homolog thereof having at least 55% identity to and at least 55% coverage of, the amino acid sequence set forth in any one of SEQ ID NOS: 126, 31, 33, 35, 37, 39, 41, 66, 81, 94, 96, 98, 100, 102, or 104. In some embodiments, the amino acid sequence of said CSLG enzyme is set forth in any one of SEQ ID NOS: 126, 31, 33, 35, 37, 39, 41, 66, 81, 94, 96, 98, 100, 102, or 104; or a homolog thereof having at least 55% identity to and at least 80% coverage of, the amino acid sequence set forth in any one of SEQ ID NOS: 126, 31, 33, 35, 37, 39, 41, 66, 81, 94, 96, 98, 100, 102, or 104.

In some embodiments, the amino acid sequence of said CSLG enzyme is set forth in any one of SEQ ID NOS: 126, 31, 33, 35, 37, 39, 41, 66, 81, 94, 96, 98, 100, 102, or 104; or a homolog thereof having at least 80% identity to and at least 80% coverage of, the amino acid sequence set forth in any one of SEQ ID NOS: 126, 31, 33, 35, 37, 39, 41, 66, 81, 94, 96, 98, 100, 102, or 104.

In some embodiments, the altered expression of the at least one cellulose synthase like G (CSLG) gene is altered by introducing (1) one or more point mutations into the nucleic acid sequence, (2) deletions within the nucleic acid sequence, or (3) insertions within the nucleic acid, any combination thereof, or (4) any combination thereof, wherein said introducing comprising mutagenizing coding or non-coding sequence.

In some embodiments, the altered expression of the at least one CSLG gene is altered by introducing a silencing molecule targeted to the at least one CSLG gene and wherein: (a) when the nucleic acid of the CSLG is set forth in SEQ ID NO: 30, the silencing molecule is set forth in SEQ ID NO: 42 or a complementary sequence thereof, or a homolog thereof having at least 80% identity to and at least 80% coverage of, the nucleic acid sequence set forth in SEQ ID NO: 42 or a complementary sequence thereof, (b) when the nucleic acid of the CSLG is set forth in SEQ ID NO: 34, the silencing molecule is set forth in SEQ ID NO: 43 or a complementary sequence thereof, or a homolog thereof having at least 80% identity to and at least 80% coverage of, the nucleic acid sequence set forth in SEQ ID NO: 43 or a complementary sequence thereof; (c) when the nucleic acid of the CSLG is set forth in SEQ ID NO: 38, the silencing molecule is set forth in SEQ ID NO: 44 or a complementary sequence thereof, or a homolog thereof having at least 80% identity to and at least 80% coverage of, the nucleic acid sequence set forth in SEQ ID NO: 44 or a complementary sequence thereof, (d) when the nucleic acid of the CSLG is set forth in SEQ ID NO: 65 or SEQ ID NO: 93, the silencing molecule is set forth in SEQ ID NO: 106 or a complementary sequence thereof, or a homolog thereof having at least 80% identity to and at least 80% coverage of, the nucleic acid sequence set forth in SEQ ID NO: 106 or a complementary sequence thereof; or (e) when the nucleic acid of the CSLG is set forth in SEQ ID NO: 95, the silencing molecule is set forth in SEQ ID NO: 107 or a complementary sequence thereof, or a homolog thereof having at least 80% identity to and at least 80% coverage of, the nucleic acid sequence set forth in SEQ ID NO: 107 or a complementary sequence thereof.

The triterpenoid saponin or said derivative, metabolite, or biosynthetic intermediate thereof, may have a broad range of commercial uses and may in some embodiments comprise a sweetener, foaming agent, emulsifier, preservative, anti-carcinogen, hypocholesterolemic agent, anti-inflammatory agent, anti-oxidant, biological adjuvant, anti-microbial agent, insecticidal agent, antifeedant, anti-fungal agent, or any combination thereof.

In some embodiments, (a) said steroidal glycoalkaloid comprises alpha-tomatine, tomatine, dehydrotomatine, hydroxytomatine, acetoxytomatine, di-hydroxytomatine, alpha-chaconine, alpha-solanine, alpha-solasonine, alpha-solmargine, or their derivatives and pathway intermediates or any combination thereof, (b) said steroidal saponin comprises _uttroside B, tomatosides, and all related compounds, or their derivatives and pathway intermediates, or any combination thereof, (c) said triterpenoid saponin comprises medicagenic acid 3-O-glucuronide (MA-3-GlcA) (Compound 6), Compound 7, Compound 8, Compound 9, Compound 10, Compound 11, glycyrrhizin (Compound 14), glycyrrhetinic acid 3-O-monoglucuronide (Compound 15), bayogenin (Compound 25), bayogenin-hexA-hex-hex (Compound 31), serjanic acid (Compound 26), serjanic acid-hexA-hex (Compound 32), soyasapogenol A (Compound 29), soyasapogenol B (Compound 30), soyasapogenol A-hexA-hex-pent (Compound 34), soyasaponin VI (Compound 35), soyasaponin I, betavulgaroside IV (Compound 33), hederagenin-3GlcA, gypsogenin-3GlcA, gypsogenic acid-3GlcA, or a QS-21 adjuvant, or any combination thereof, (d) the biosynthetic intermediate of said triterpenoid saponin comprises Compound 1, Compound 2, medicagenic acid (Compound 5), oleanolic acid (Compound 3), oleanolic acid-3GlcA, augustic acid (Compound 4), augustic acid-3GlcA, or glycyrrhetinic acid (Compound 13), or any combination thereof, or (e) any combination of these.

In some embodiments, the genetically modified plant has an altered content of a phytosterol, or a derivative thereof, a metabolite thereof, or a biosynthetic intermediate thereof, a cholesterol, or a derivative thereof, a metabolite thereof, or a biosynthetic intermediate thereof, a phytocholesterol, or a derivative thereof, a metabolite thereof, or a biosynthetic intermediate thereof, a cholestenol, or a derivative thereof, a metabolite thereof, or a biosynthetic intermediate thereof, or a phytocholestenol, or a derivative thereof, a metabolite thereof, or a biosynthetic intermediate thereof.

In some embodiments, the genetically modified plant has an altered content of a phytosterol, or a derivative thereof, a metabolite thereof, or a biosynthetic intermediate thereof, a cholesterol, or a derivative thereof, a metabolite thereof, or a biosynthetic intermediate thereof; a phytocholesterol, or a derivative thereof, a metabolite thereof, or a biosynthetic intermediate thereof, a cholestenol, or a derivative thereof, a metabolite thereof, or a biosynthetic intermediate thereof, or a phytocholestenol, or a derivative thereof, a metabolite thereof, or a biosynthetic intermediate thereof, wherein: (a) when the altered content of the at least one steroidal alkaloid, a derivative thereof, a metabolite thereof, or a biosynthetic intermediate thereof, of the at least one steroidal saponin, a derivative thereof, a metabolite thereof, or a biosynthetic intermediate thereof, or of the at least one triterpenoid saponin, a derivative thereof, a metabolite thereof, or a biosynthetic intermediate thereof comprises an increased amount compared to the corresponding unmodified plant, the altered content of the phytosterol, or a derivative thereof, a metabolite thereof, or a biosynthetic intermediate thereof, the cholesterol, or a derivative thereof, a metabolite thereof, or a biosynthetic intermediate thereof, the phytocholesterol, or a derivative thereof, a metabolite thereof, or a biosynthetic intermediate thereof; the cholestenol, or a derivative thereof, a metabolite thereof, or a biosynthetic intermediate thereof, or the phytocholestenol, or comprises a reduced amount compared to the corresponding unmodified plant a derivative thereof, a metabolite thereof, or a biosynthetic intermediate thereof, or (b) when the altered content of the at least one steroidal alkaloid, a derivative thereof, a metabolite thereof, or a biosynthetic intermediate thereof, of the at least one steroidal saponin, a derivative thereof, a metabolite thereof, or a biosynthetic intermediate thereof, or of the at least one triterpenoid saponin, a derivative thereof, a metabolite thereof, or a biosynthetic intermediate thereof comprises a reduced amount compared to the corresponding unmodified plant, the altered content of the phytosterol, or a derivative thereof, a metabolite thereof, or a biosynthetic intermediate thereof, the cholesterol, or a derivative thereof, a metabolite thereof, or a biosynthetic intermediate thereof, the phytocholesterol, or a derivative thereof, a metabolite thereof, or a biosynthetic intermediate thereof; the cholestenol, or a derivative thereof, a metabolite thereof, or a biosynthetic intermediate thereof, or the phytocholestenol, or a derivative thereof, a metabolite thereof, or a biosynthetic intermediate thereof comprises an increased amount compared to the corresponding unmodified plant.

In certain embodiments, an increase in each or sterodial saponins, steroidal alakoids, and or triterpenoid saponins may cause a change in the content level of the other(s). In some embodiments, the change comprises an increased content. In some embodiments, the change comprises a decrease content. In some embodiments, the change comprises an increased content of one and a decrease content of another.

In some embodiments, said plant comprises a plant in the Poales order, the Caryophyllales order, the Solanales order, the Fabales order, the Malvales order, the Apiales order, or the Brassicales order, the Asparagales order, the Dioscoreales order, or the Liliales order.

In some embodiments, (a) when said plant comprises a plant in the Poales order, said plant is selected from the group of genera consisting of the Oryza genus, the Hordeum genus, the Avena genus, and the Triticum genus; (b) when said plant comprises a plant in the Caryophyllales order, said plant is selected from the group of genera consisting of the Spinacia genus, the Chenopodium genus, the Beta genus, the Rheum genus, the Vaccaria genus, the Saponaria genus, and the Gypsophila genus; (c) when said plant comprises a plant in the Solanales order, said plant is selected from the group of genera consisting of the Solanum genus, the Capsicum genus, the Nicotiana genus, the Hyoscyamus genus, the Datura genus, and the Atropa genus; (d) when said plant comprises a plant in the Fabales order, said plant is selected from the group of genera consisting of the Glycyrrhiza genus, the Medicago genus, the Glycine genus, the Lotus genus, the Cicer genus, the Phaseolus genus, the Pisum genus, the Arachis genus, the Lupinus genus, and the Acacia genus; (e) when said plant comprises a plant in the Malvales order, said plant is selected from the Theobroma genus; (f) when said plant comprises a plant in the Apiales order, said plant is selected from the group of genera consisting of the Daucus genus, the Apium genus, the Petroselinum genus, the Panax genus, the Bupleurum genus, the Hedera genus, and the Centella genus; or (g) when said plant comprises a plant in the Brassicales order, said plant is selected from the group of genera consisting of the Arabidopsis genus, the Brassica genus, the Capparis genus, and the Carica genus.

In some embodiments, (a) when said plant comprises a plant in the Caryophyllales order, said plant is selected from the group consisting of spinach, beetroot, and quinoa; (b) when said plant comprises a plant in the Solanales order, said plant is selected from the group consisting of tomato, wild tomato, potato, wild potato, eggplant, pepper, bell pepper, cayenne pepper, chili pepper, pimiento, tabasco pepper, ground cherry, tobacco, and bittersweet; or (c) when said plant comprises a plant in the Fabales order, said plant is selected from the group consisting of peas, alfalfa, soy, Lotus japonicus, and licorice.

In some embodiments, the plant is a tomato plant having: (i) an increased content of alpha-tomatine or a derivative thereof, a metabolite thereof, or a biosynthetic intermediate thereof, or tomatidine or a derivative thereof, a metabolite thereof, or a biosynthetic intermediate thereof, or a steroidal saponin or a derivative thereof, a metabolite thereof, or a biosynthetic intermediate thereof, or (ii) an altered content of a phytosterol or a derivative thereof, a metabolite thereof, or a biosynthetic intermediate thereof, a cholesterol or a derivative thereof, a metabolite thereof, or a biosynthetic intermediate thereof, a phytocholesterol or a derivative thereof, a metabolite thereof, or a biosynthetic intermediate thereof, a cholestenol or a derivative thereof, a metabolite thereof, or a biosynthetic intermediate thereof, or a phytocholestenol or a derivative thereof, a metabolite thereof, or a biosynthetic intermediate thereof.

In some embodiments, the plant is a tomato plant having: (i) an increased content of alpha-tomatine or a derivative thereof, a metabolite thereof, or a biosynthetic intermediate thereof, or tomatidine or a derivative thereof, a metabolite thereof, or a biosynthetic intermediate thereof, or a steroidal saponin or a derivative thereof, a metabolite thereof, or a biosynthetic intermediate thereof, or (ii) a reduced content of a phytosterol or a derivative thereof, a metabolite thereof, or a biosynthetic intermediate thereof, a cholesterol or a derivative thereof, a metabolite thereof, or a biosynthetic intermediate thereof, a phytocholesterol or a derivative thereof, a metabolite thereof, or a biosynthetic intermediate thereof, a cholestenol or a derivative thereof, a metabolite thereof, or a biosynthetic intermediate thereof, or a phytocholestenol or a derivative thereof, a metabolite thereof, or a biosynthetic intermediate thereof.

In some embodiments, the plant is a potato plant having: (i) an increased content of alpha-chaconine or a derivative thereof, a metabolite thereof, or a biosynthetic intermediate thereof, or alpha-solanine or a derivative thereof, a metabolite thereof, or a biosynthetic intermediate thereof, or a steroidal saponin or a derivative thereof, a metabolite thereof, or a biosynthetic intermediate thereof, or (ii) an altered content of a phytosterol or a derivative thereof, a metabolite thereof, or a biosynthetic intermediate thereof; a cholesterol or a derivative thereof, a metabolite thereof, or a biosynthetic intermediate thereof, a phytocholesterol or a derivative thereof, a metabolite thereof, or a biosynthetic intermediate thereof, a cholestenol or a derivative thereof, a metabolite thereof, or a biosynthetic intermediate thereof, or a phytocholestenol or a derivative thereof, a metabolite thereof, or a biosynthetic intermediate thereof.

In some embodiments, the plant is a potato plant having: (i) an increased content of alpha-chaconine or a derivative thereof, a metabolite thereof, or a biosynthetic intermediate thereof, or alpha-solanine or a derivative thereof, a metabolite thereof, or a biosynthetic intermediate thereof, or a steroidal saponin or a derivative thereof, a metabolite thereof, or a biosynthetic intermediate thereof, or (ii) a reduced content of a phytosterol or a derivative thereof, a metabolite thereof, or a biosynthetic intermediate thereof; a cholesterol or a derivative thereof, a metabolite thereof, or a biosynthetic intermediate thereof, a phytocholesterol or a derivative thereof, a metabolite thereof, or a biosynthetic intermediate thereof, a cholestenol or a derivative thereof, a metabolite thereof, or a biosynthetic intermediate thereof, or a phytocholestenol or a derivative thereof, a metabolite thereof, or a biosynthetic intermediate thereof.

In some embodiments, the plant is an eggplant plant having: (i) an increased content of alpha-solasonine or a derivative thereof, a metabolite thereof, or a biosynthetic intermediate thereof, or alpha-solamargine or a derivative thereof, a metabolite thereof, or a biosynthetic intermediate thereof, or a steroidal saponin or a derivative thereof, a metabolite thereof, or a biosynthetic intermediate thereof, or (ii) an altered content of a phytosterol or a derivative thereof, a metabolite thereof, or a biosynthetic intermediate thereof, a cholesterol or a derivative thereof, a metabolite thereof, or a biosynthetic intermediate thereof, a phytocholesterol or a derivative thereof, a metabolite thereof, or a biosynthetic intermediate thereof, a cholestenol or a derivative thereof, a metabolite thereof, or a biosynthetic intermediate thereof, or a phytocholestenol or a derivative thereof, a metabolite thereof, or a biosynthetic intermediate thereof.

In some embodiments, the plant is an eggplant plant having: (i) an increased content of alpha-solasonine or a derivative thereof, a metabolite thereof, or a biosynthetic intermediate thereof, or alpha-solamargine or a derivative thereof, a metabolite thereof, or a biosynthetic intermediate thereof, or a steroidal saponin or a derivative thereof, a metabolite thereof, or a biosynthetic intermediate thereof, or (ii) a reduced content of a phytosterol or a derivative thereof, a metabolite thereof, or a biosynthetic intermediate thereof, a cholesterol or a derivative thereof, a metabolite thereof, or a biosynthetic intermediate thereof, a phytocholesterol or a derivative thereof, a metabolite thereof, or a biosynthetic intermediate thereof, a cholestenol or a derivative thereof, a metabolite thereof, or a biosynthetic intermediate thereof, or a phytocholestenol or a derivative thereof, a metabolite thereof, or a biosynthetic intermediate thereof.

In some embodiments, said plant cell comprises a leaf cell, a petiole cell, a plant stem or stalk cell, a root cell, a bud cell, a tuber cell, a bean cell, a grain or kernel cell, a fruit cell, a nut cell, a legume cell, a seed or seed cell, a callus cells, a bract cell, a callus cell, and a flower cell.

Specifically disclosed herein are methods of producing steroidal alkaloids, steroidal saponins, or triterpenoid saponins in a genetically modified cell, the methods comprising: (a) introducing an at least one heterologous gene into said cell, said at least one heterologous gene encoding a cellulose synthase like G (CSLG) enzyme, wherein said heterologous gene is optionally comprised in a vector; and (b) expressing said at least one heterologous gene in said cell; wherein said cell comprises an increased content of at least one steroidal alkaloid, at least one steroidal saponin, or at least one triterpenoid saponin compared to a corresponding unmodified cell.

In some embodiments, said introducing further comprises introducing an at least one additional heterologous gene into said cell, said heterologous gene selected from the group consisting of the group encoding a saponin beta-amyrin synthase, a cytochrome P450, a glycosyltransferase, an acyltransferase, and a UDP-glucose 6-dehydrogenase I, or any combination thereof, wherein said at least one additional heterologous gene is optionally comprised in a vector; and further comprising expressing said at least one additional heterologous gene in said cell.

In some embodiments, (a) said at least one heterologous gene is operably linked to a promoter, a transcription termination sequence, or a combination thereof; or (b) said at least one additional heterologous gene is operably linked to a promoter, a transcription termination sequence, or a combination thereof, or (c) a combination thereof of (a) and (b).

In some embodiments, said introducing comprises transforming said at least one cell with (a)
said at least one heterologous gene or a polynucleotide sequence encoding said at least one heterologous gene, or the vector comprising said at least one heterologous gene; or (b) said at least one additional heterologous gene or a polynucleotide sequences encoding said at least one additional heterologous gene, or the vector comprising said at least one additional heterologous gene; or (c)
a combination thereof of (a) and (b); wherein said expressing comprises transient expression or constitutive expression.

Specifically disclosed herein are methods of reducing the content of at least one steroidal alkaloid, a derivative thereof, a metabolite thereof, or a biosynthetic intermediate thereof, of at least one steroidal saponin, a derivative thereof, a metabolite thereof, or a biosynthetic intermediate thereof, or of at least one triterpenoid saponin, a derivative thereof, a metabolite thereof, or a biosynthetic intermediate thereof, in at least one cell of a plant or a plant part, the method comprising genetically modifying said at least one plant cell, said genetic modification comprising: (a) transforming said at least one plant cell with at least one silencing molecule targeted to a nucleic acid gene sequence encoding a Cellulose Synthase Like G (CSLG) enzyme; or (b) mutagenizing at least one nucleic acid sequence encoding a Cellulose Synthase Like G (CSLG) enzyme, wherein the mutagenesis comprises introducing (1) one or more point mutations into the nucleic acid sequence, (2) deletions within the nucleic acid sequence, or (3) insertions within the nucleic acid, or (4) any combination thereof, wherein said introducing comprising mutagenizing coding or non-coding sequence; wherein expression of the gene encoding the CSLG enzyme is reduced in the genetically modified plant cell compared to its expression in a corresponding unmodified plant cell, wherein the plant comprising said genetically modified cell comprises reduced content of at least one steroidal alkaloid, a derivative thereof, a metabolite thereof, or a biosynthetic intermediate thereof, of at least one steroidal saponin, a derivative thereof, a metabolite thereof, or a biosynthetic intermediate thereof, or of at least one triterpenoid saponin, a derivative thereof, a metabolite thereof, or a biosynthetic intermediate thereof, compared to the corresponding unmodified plant.

In some embodiments: (a) when the nucleic acid of the CSLG is set forth in SEQ ID NO: 30, the silencing molecule is set forth in SEQ ID NO: 42 or a complementary sequence thereof, or a homolog thereof having at least 80% identity to and at least 80% coverage of, the nucleic acid sequence set forth in SEQ ID NO: 42 or a complementary sequence thereof, (b) when the nucleic acid of the CSLG is set forth in SEQ ID NO: 34, the silencing molecule is set forth in SEQ ID NO: 43 or a complementary sequence thereof, or a homolog thereof having at least 80% identity to and at least 80% coverage of, the nucleic acid sequence set forth in SEQ ID NO: 43 or a complementary sequence thereof, (c) when the nucleic acid of the CSLG is set forth in SEQ ID NO: 38, the silencing molecule is set forth in SEQ ID NO: 44 or a complementary sequence thereof, or a homolog thereof having at least 80% identity to and at least 80% coverage of, the nucleic acid sequence set forth in SEQ ID NO: 44 or a complementary sequence thereof, (d) when the nucleic acid of the CSLG is set forth in SEQ ID NO: 65 or SEQ ID NO: 93, the silencing molecule is set forth in SEQ ID NO: 106 or a complementary sequence thereof, or a homolog thereof having at least 80% identity to and at least 80% coverage of, the nucleic acid sequence set forth in SEQ ID NO: 106 or a complementary sequence thereof, or (e) when the nucleic acid of the CSLG is set forth in SEQ ID NO: 95, the silencing molecule is set forth in SEQ ID NO: 107 or a complementary sequence thereof, or a homolog thereof having at least 80% identity to and at least 80% coverage of, the nucleic acid sequence set forth in SEQ ID NO: 107 or a complementary sequence thereof.

In some embodiments: (a) said steroidal alkaloid, said steroidal saponin, or said triterpenoid saponin comprises a toxin or a bitter tasting compound or having hormone mimicking properties, or a combination thereof, or (b) said steroidal glycoalkaloid comprises alpha-tomatine, tomatine, alpha-chaconine, alpha-solanine, alpha-solasonine, alpha-solmargine, or any combination thereof, or (c) said steroidal saponin comprises utteroside B and tonatosides and related compounds (all steroidal saponins family), pathway intermediates and derivatives, or any combination thereof, or (d) said triterpenoid saponin comprises medicagenic acid 3-O-glucuronide (MA-3-GlcA) (Compound 6), Compound 7, Compound 8, Compound 9, Compound 10, Compound 11, glycyrrhizin (Compound 14), Glycyrrhetinic acid 3-O-monoglucuronide (compound 15), bayogenin (Compound 25), bayogenin-hexA-hex-hex (Compound 31), serjanic acid (Compound 26), serjanic acid-hexA-hex (Compound 32), soyasapogenol A (Compound 29), soyasapogenol B (Compound 30), soyasapogenol A-hexA-hex-pent (Compound 34), soyasaponin VI (Compound 35), soyasaponin I, betavulgaroside IV (Compound 33), hederagenin-3GlcA, gypsogenin-3GlcA, gypsogenic acid-3GlcA, or a QS-21 adjuvant, or any combination thereof, or (e) said intermediate comprises Compound 1, Compound 2, medicagenic acid (Compound 5), oleanolic acid (Compound 3), oleanolic acid-3GlcA, augustic acid (Compound 4), augustic acid-3GlcA, or glycyrrhetinic acid (Compound 13), or any combination thereof, or (f) a combination thereof.

In certain embodiments, reduction of a triterpenoid saponin is desired, for example, but not limited to triterpenoid saponins having a bitter taste, as in quinoa, or being toxic. Other examples of triterpenoid saponins wherein reduction may be desired include triterpenoid saponins that have toxic properties or mimic hormones. Thus, in certain embodiments, increased production of triterpenoid saponins is beneficial and in other embodiments, decreased production of triterpenoid saponins is beneficial.

In some embodiments, the method further comprises altering the content of a phytosterol, or a derivative thereof, a metabolite thereof, or a biosynthetic intermediate thereof, a cholesterol, or a derivative thereof, a metabolite thereof, or a biosynthetic intermediate thereof, a phytocholesterol, or a derivative thereof, a metabolite thereof, or a biosynthetic intermediate thereof, a cholestenol, or a derivative thereof, a metabolite thereof, or a biosynthetic intermediate thereof, or a phytocholestenol, or a derivative thereof, a metabolite thereof, or a biosynthetic intermediate thereof.

In some embodiments, the method further comprises increasing the content of a phytosterol, or a derivative thereof, a metabolite thereof, or a biosynthetic intermediate thereof, a cholesterol, or a derivative thereof, a metabolite thereof, or a biosynthetic intermediate thereof, a phytocholesterol, or a derivative thereof, a metabolite thereof, or a biosynthetic intermediate thereof, a cholestenol, or a derivative thereof, a metabolite thereof, or a biosynthetic intermediate thereof, or a phytocholestenol, or a derivative thereof, a metabolite thereof, or a biosynthetic intermediate thereof.

According to some embodiments, the method further comprises purifying the phytosterol, or derivative, metabolite, or biosynthetic intermediate thereof, the cholesterol, or derivative, metabolite, or biosynthetic intermediate thereof; the phytocholesterol, or derivative, metabolite, or biosynthetic intermediate thereof, the cholestenol, or derivative, metabolite, or biosynthetic intermediate; or the phytocholestenol, or derivative, metabolite, or biosynthetic intermediate thereof, extracted from the transformed plant. According to certain embodiments, the phytosterol comprises phytocholesterol. According to some embodiments, the phytosterol, or derivative, metabolite, or biosynthetic intermediate thereof, the cholesterol, or derivative, metabolite, or biosynthetic intermediate thereof; the phytocholesterol, or derivative, metabolite, or biosynthetic intermediate thereof, the cholestenol, or derivative, metabolite, or biosynthetic intermediate; or the phytocholestenol, or derivative, metabolite, or biosynthetic intermediate thereof, extracted from the transformed plant, comprises a nutrient or food additive, a high-value steroidal compound (e.g., pro-vitamin D and/or diosgenin), an anti-cholesterol agent (e.g., a plant phytosterol competing with dietary cholesterol in a mammalian or avian gut), or a cosmetic agent.

Specifically disclosed herein are methods of increasing the content of at least one steroidal alkaloid, a derivative thereof, a metabolite thereof, or a biosynthetic intermediate thereof, of at least one steroidal saponin, a derivative thereof, a metabolite thereof, or a biosynthetic intermediate thereof, or of at least one triterpenoid saponin, a derivative thereof, a metabolite thereof, or a biosynthetic intermediate thereof, in at least one cell of a plant or plant part, the method comprising genetically modifying said at least one plant cell, said genetic modification comprising: (a) mutagenizing at least one nucleic acid sequence encoding a Cellulose Synthase Like G (CSLG) enzyme, wherein the mutagenesis comprises introducing (1) one or more point mutations into the nucleic acid sequence, (2) deletions within the nucleic acid sequence, or (3) insertions within the nucleic acid, or (4) any combination thereof, wherein said introducing comprising mutagenizing coding or non-coding sequence; and (b) expressing said nucleic acid encoding said CSLG; wherein the plant comprising said genetically modified cell comprises increased content of at least one steroidal alkaloid, a derivative thereof, a metabolite thereof, or a biosynthetic intermediate thereof, of at least one steroidal saponin, a derivative thereof, a metabolite thereof, or a biosynthetic intermediate thereof, or of at least one triterpenoid saponin, a derivative thereof, a metabolite thereof, or a biosynthetic intermediate thereof, compared to the corresponding unmodified plant.

In some embodiments: (a) said steroidal alkaloid, said steroidal saponin, or said triterpenoid saponin comprises a toxin or a bitter tasting compound or having hormone mimicking properties, or a combination thereof, or (b) said steroidal glycoalkaloid comprises alpha-tomatine, tomatine, alpha-chaconine, alpha-solanine, alpha-solasonine, alpha-solmargine, or any combination thereof, or (c) said steroidal saponin comprises uttroside B, a tomatoside, or any combination thereof; or (d) said triterpenoid saponin comprises medicagenic acid 3-O-glucuronide (MA-3-GlcA) (Compound 6), Compound 7, Compound 8, Compound 9, Compound 10, Compound 11, glycyrrhizin (Compound 14), Glycyrrhetinic acid 3-O-monoglucuronide (compound 15), bayogenin (Compound 25), bayogenin-hexA-hex-hex (Compound 31), serjanic acid (Compound 26), serjanic acid-hexA-hex (Compound 32), soyasapogenol A (Compound 29), soyasapogenol B (Compound 30), soyasapogenol A-hexA-hex-pent (Compound 34), soyasaponin VI (Compound 35), soyasaponin I, betavulgaroside IV (Compound 33), hederagenin-3GlcA, gypsogenin-3GlcA, gypsogenic acid-3GlcA, or a QS-21 adjuvant, or any combination thereof, or (e) said intermediate comprises Compound 1, Compound 2, medicagenic acid (Compound 5), oleanolic acid (Compound 3), oleanolic acid-3GlcA, augustic acid (Compound 4), augustic acid-3GlcA, or glycyrrhetinic acid (Compound 13), or any combination thereof, or (f) a combination thereof.

In some embodiments: (a) expression of the gene encoding the CSLG enzyme is increased in the genetically modified plant cell compared to its expression in a corresponding unmodified plant cell; or (b) said encoded CSLG enzyme has increased activity in the genetically modified plant cell compared to its activity in a corresponding unmodified plant cell; or (c) said encoded CSLG enzyme has increased stability in the genetically modified plant cell compared to its stability in a corresponding unmodified plant cell; or (d) any combination thereof of (a), (b), and (c).

In some embodiments, the method further comprises altering the content of a phytosterol, or a derivative thereof, a metabolite thereof, or a biosynthetic intermediate thereof, a cholesterol, or a derivative thereof, a metabolite thereof, or a biosynthetic intermediate thereof, a phytocholesterol, or a derivative thereof, a metabolite thereof, or a biosynthetic intermediate thereof, a cholestenol, or a derivative thereof, a metabolite thereof, or a biosynthetic intermediate thereof, or a phytocholestenol, or a derivative thereof, a metabolite thereof, or a biosynthetic intermediate thereof.

In some embodiments, the method further comprises reducing the content of a phytosterol, or a derivative thereof, a metabolite thereof, or a biosynthetic intermediate thereof, a cholesterol, or a derivative thereof, a metabolite thereof, or a biosynthetic intermediate thereof; a phytocholesterol, or a derivative thereof, a metabolite thereof, or a biosynthetic intermediate thereof, a cholestenol, or a derivative thereof, a metabolite thereof, or a biosynthetic intermediate thereof, or a phytocholestenol, or a derivative thereof, a metabolite thereof, or a biosynthetic intermediate thereof.

Each possibility described herein represents a separate embodiment of the invention.

### Genetically Modified Cells & Genetically Modified Plants

Disclosed herein are genetically modified cells and genetically modified plants, wherein expression of key genes in the steroidal glycoalkaloid/steroidal saponin/triterpenoid saponin metabolic pathway (biosynthesis pathway of steroidal alkaloids, steroidal saponins, and triterpenoid saponins) has been altered. Altering the expression of these genes results in concomitant alteration in the steroidal alkaloid profile, in the steroidal saponin profile, or in the triterpenoid profile. In an non-limiting example, changing the production level of steroidal alkaloid can result in improved plants comprising elevated content of steroidal alkaloids having increased resistance to pathogens, or plants having a reduced content of these secondary compounds in the plant edible parts and thus producing improved crops, wherein the improved crop has reduced or eliminated anti-nutritional content. Alternatively, or additionally, in another non-limiting example, controlling the expression of genes disclosed herein may be used for the production of desired steroidal alkaloids or plant-based cholesterol for further use, e.g., in the nutritional, cosmetic, or pharmaceutical industry. In another non-limiting example, disclosed herein are the means and methods for producing crop plants of the Solanales order and other orders that are devoid of toxic amounts of deleterious steroidal alkaloids typically present in edible parts of these plants. Thus, the cells and plants disclosed herein are of significant nutritional and commercial value.

Disclosed herein are an array of co-expressed genes that participate in the biosynthesis pathway of steroidal alkaloids, steroidal saponins, or triterpenoid saponins. The present invention further discloses key genes in this pathway, altering the expression of which result in concomitant alteration in the steroidal alkaloid profile, in the steroidal saponin profile, or in the triterpenoid profile. In one non-limiting example, changing the production level of steroidal alkaloids can result in an improved plant comprising elevated content of steroidal alkaloids having increased resistance to pathogens, or plants having a reduced content of these secondary compounds in the plant edible parts and thus producing improved crops. Alternatively, or additionally, in one non-limiting example, controlling the expression of genes revealed in the present invention can be used, e.g., for the production of desired steroidal alkaloids or plant-based cholesterol or other phytosterols for further use, for example, in the nutrition, cosmetic, or pharmaceutical industries. Thus, the cells and plants of the present invention are thus of significant nutritional, cosmetic, pharmaceutical, and commercial value.

Disclosed herein are genetically modified cells (e.g., plant cells, yeast cells, algal cells, bacterial cells, insect cells) and plants comprising at least one genetically modified cell, wherein the genetically modified cells are expressing at least one heterologous gene encoding an enzyme within the steroidal alkaloid/steroidal saponin/triterpenoid saponin biosynthetic pathway. These enzymes include, in some embodiments, cellulose synthase like G (CSLG) enzymes, as well as saponin beta-amyrin synthases, cytochrome P450s, glycosyltransferases, and acyltransferases.

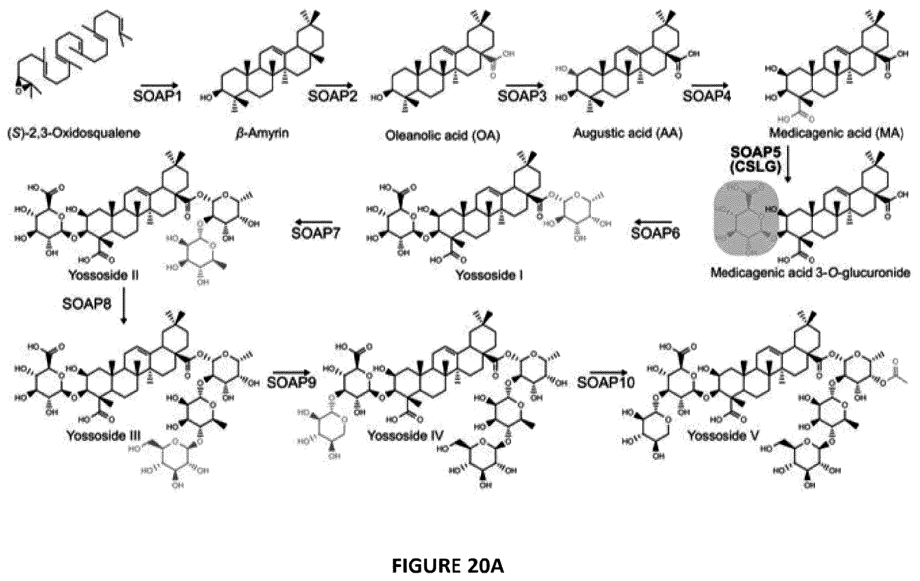

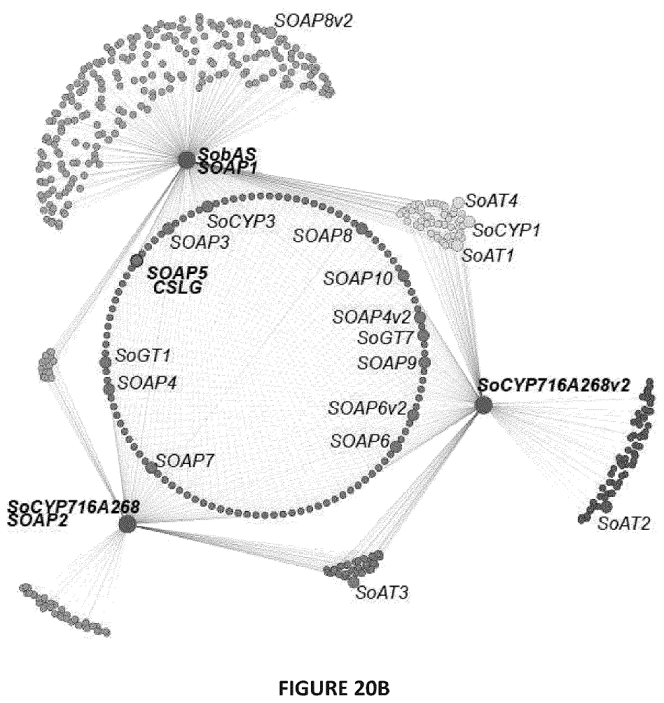

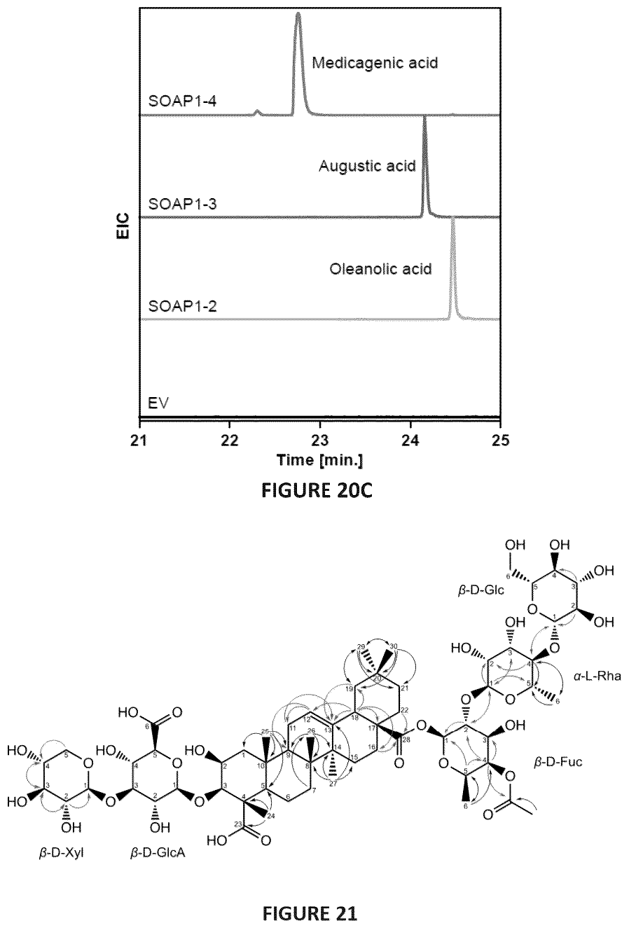

In some embodiments, a genetically modified cell disclosed herein, expresses at least one heterologous gene encoding an enzyme, said enzyme selected from the group consisting of a saponin beta-amyrin synthase, a cytochrome P450, a glycosyltransferase, an acyltransferase, a UDP-glucose 6-dehydrogenase 1, and a cellulose synthase like G (CSLG). FIG. 1 provides a non-limiting example of steroidal alkaloids and steroidal saponins in the triterpenoid biosynthetic pathway in Solanaceous plants (here, tomato) beginning with cholesterol, wherein a skilled artisan would recognize that the pathway may encompass production of saponin aglycones and glycosylated steroidal alkaloids biosynthetic intermediates and glycosylated saponins biosynthetic intermediates, and results in the production of a steroidal alkaloid (e.g., alpha-tomatine) or a steroidal saponin (e.g., uttroside B). FIG. 20A provides a non-limiting example of a triterpenoid saponin pathway in spinach, wherein a skilled artisan would recognize that the pathway may encompass production of saponin aglycones and glycosylated saponins biosynthetic intermediates, and results in the production of a triterpenoid saponin (Yossoside V; Compound 11).

Altering the expression of at least one of the genes in the steroidal alkaloid/steroidal saponin/triterpenoid saponin biosynthetic pathway, in some embodiments, results in concomitant alteration in the steroidal alkaloid profile or intermediates of that pathway, in the steroidal saponin profile or intermediates of that pathway, or in the triterpenoid saponin profile or intermediates of that pathway. In some embodiments, the altered expression in a genetically modified cells comprises increased expression compared with a corresponding unmodified cell. In some embodiments, increased expression comprises de novo expression from a gene encoding an enzyme not previously present in the cell. In some embodiments, increased expression comprises an increase of expression of a gene already present within the cells. In some embodiments, increased expression results in an increased amount of the encoded enzyme.

In some embodiments, the alteration in the steroidal alkaloid profile comprising an increase in at least one steroidal alkaloid or an intermediate thereof. In some embodiments, the alteration in the steroidal alkaloid profile comprising a decrease in at least one steroidal alkaloid or an intermediate thereof.

In some embodiments, the alteration in the steroidal saponin profile comprising an increase in at least one steroidal saponin or an intermediate thereof. In some embodiments, the alteration in the steroidal saponin profile comprising a decrease in at least one steroidal saponin or an intermediate thereof.

In some embodiments, the alteration in the triterpenoid saponin profile comprising an increase in at least one triterpenoid saponin or an intermediate thereof. In some embodiments, the alteration in the triterpenoid saponin profile comprising a decrease in at least one triterpenoid saponin or an intermediate thereof.

In some embodiments, introducing a nucleotide sequence encoding at least one of the enzymes of the steroidal alkaloid/steroidal saponin/triterpenoid saponin biosynthetic pathway into a heterologous cell, (for example but not limited to a plant cell, an algal cell, a yeast cell, an insect cell, or a bacterial cell) or an organism (for example but not limited to a plant), results in increased expression of the at least one enzyme and increased production of a steroidal alkaloid, a steroidal saponin, or a triterpenoid saponin. In some embodiments, introducing a nucleotide sequence encoding at least one of the enzymes of the steroidal alkaloid/steroidal saponin/triterpenoid saponin biosynthetic pathway into a heterologous cell or organism, results in production of a steroidal alkaloid, a steroidal saponin, or a triterpenoid saponin not naturally produced by the heterologous cell (for example but not limited to a plant cell, an algal cell, a yeast cell, an insect cell, or a bacterial cell) or organism (for example but not limited to a plant).

Throughout described herein are genetically modified cells and uses thereof. In some embodiments, a genetically modified cell is selected from any of an algal cell, a yeast cell, an insect cell, or a bacterial cell. One skilled in the art would know how to use the genes and nucleic acid sequences disclosed herein to genetically modified a eukaryotic cell, for example mammalian cells, or a prokaryotic cell, for example bacterium, to produce at least one steroidal alkaloid, steroidal saponin, or triterpenoid saponin. Further, one skilled in the art could use the genes and nucleic acid sequences disclosed herein to produce at least one steroidal alkaloid, steroidal saponin, or triterpenoid saponin in a cell-free in vitro system. In some embodiments, the genetically modified cell is a plant cell, and said plant cell is used to produce at least one steroidal alkaloid, steroidal saponin, or triterpenoid saponin. In some embodiments, the genetically modified cell is a plant cell or a yeast cell and said plant cell or yeast is used to produce at least one steroidal alkaloid, steroidal saponin, or triterpenoid saponin. In some embodiments, the genetically modified cell is a yeast cell, and said yeast cell is used to produce at least one steroidal alkaloid, steroidal saponin, or triterpenoid saponin. In some embodiments, the genetically modified cell is a algal cell, and said algal cell is used to produce at least one steroidal alkaloid, steroidal saponin, or triterpenoid saponin. In some embodiments, the genetically modified cell is an insect cell, and said insect cell is used to produce at least one steroidal alkaloid, steroidal saponin, or triterpenoid saponin. In some embodiments, the genetically modified cell is a bacterium, and said bacterium is used to produce at least one steroidal alkaloid, steroidal saponin, or triterpenoid saponin. In some embodiments, the genetically modified cell is a plant cell comprised in a plant or plant part and said plant cell is used to produce at least one steroidal alkaloid, steroidal saponin, or triterpenoid saponin in said plant or plant part. In some embodiments, the genetically modified cell is a plant cell comprised in a plant or plant part and said plant cell is used to reduce the production of at least one steroidal alkaloid, steroidal saponin, or triterpenoid saponin in said plant or plant part. In some embodiments, the genetically modified cell is a plant cell comprised in a plant or plant part and said plant cell is used to increase the production of at least one steroidal alkaloid, steroidal saponin, or triterpenoid saponin in said plant or plant part.

In some embodiments, heterologous expression comprises expressing an at least one heterologous plant gene from one plant species in a second plant species (see for example, Example 23 below). In some embodiments, heterologous expression comprises expressing an at least one heterologous plant gene from a plant species in a yeast cell (see for example, Example 21 below). In some embodiments, heterologous expression comprises expressing an at least one heterologous plant gene from a plant species in an algal cell. In some embodiments, heterologous expression comprises expressing an at least one heterologous plant gene from a plant species in bacterial cell. In some embodiments, heterologous expression comprises expressing an at least one heterologous plant gene from a plant species in an insect cell. In some embodiments, heterologous expression comprises expressing an at least one heterologous plant gene from a plant species in a cell of a plant, wherein the plant is of a different species.

In some embodiments, expressing at least one heterologous gene encoding an enzyme selected from a steroidal alkaloid, steroidal saponin, or triterpenoid saponin biosynthetic pathway alters the content of the steroidal alkaloid, steroidal saponin, or triterpenoid saponin produced by that pathway or alters the content of at least one biosynthetic intermediate thereof, or a combination thereof. In some embodiments, expressing more than one heterologous gene encoding at least two enzymes selected from a steroidal alkaloid, steroidal saponin, or triterpenoid saponin biosynthetic pathway alters the content of the steroidal alkaloid, steroidal saponin, or triterpenoid saponin produced by that pathway or alters the content of at least one biosynthetic intermediate thereof, or a combination thereof. In some embodiments, expressing more than one heterologous gene encoding at least three enzymes selected from a steroidal alkaloid, steroidal saponin, or triterpenoid saponin biosynthetic pathway alters the content of the steroidal alkaloid, steroidal saponin, or triterpenoid saponin produced by that pathway or alters the content of at least one biosynthetic intermediate thereof, or a combination thereof. In some embodiments, expressing more than one heterologous gene encoding at least four enzymes selected from a steroidal alkaloid, steroidal saponin, or triterpenoid saponin biosynthetic pathway alters the content of the steroidal alkaloid, steroidal saponin, or triterpenoid saponin produced by that pathway or alters the content of at least one biosynthetic intermediate thereof, or a combination thereof. In some embodiments, expressing more than one heterologous gene encoding at least five enzymes selected from a steroidal alkaloid, steroidal saponin, or triterpenoid saponin biosynthetic pathway alters the content of the steroidal alkaloid, steroidal saponin, or triterpenoid saponin produced by that pathway or alters the content of at least one biosynthetic intermediate thereof, or a combination thereof. In some embodiments, expressing more than one heterologous gene encoding at least six enzymes selected from a steroidal alkaloid, steroidal saponin, or triterpenoid saponin biosynthetic pathway alters the content of the steroidal alkaloid, steroidal saponin, or triterpenoid saponin produced by that pathway or alters the content of at least one biosynthetic intermediate thereof, or a combination thereof. In some embodiments, expressing more than one heterologous gene encoding at least seven enzymes selected from a steroidal alkaloid, steroidal saponin, or triterpenoid saponin biosynthetic pathway alters the content of the steroidal alkaloid, steroidal saponin, or triterpenoid saponin produced by that pathway or alters the content of at least one biosynthetic intermediate thereof, or a combination thereof. In some embodiments, expressing more than one heterologous gene encoding at least eight enzymes selected from a steroidal alkaloid, steroidal saponin, or triterpenoid saponin biosynthetic pathway alters the content of the steroidal alkaloid, steroidal saponin, or triterpenoid saponin produced by that pathway or alters the content of at least one biosynthetic intermediate thereof, or a combination thereof. In some embodiments, expressing more than one heterologous gene encoding at least nine enzymes selected from a steroidal alkaloid, steroidal saponin, or triterpenoid saponin biosynthetic pathway alters the content of the steroidal alkaloid, steroidal saponin, or triterpenoid saponin produced by that pathway or alters the content of at least one biosynthetic intermediate thereof, or a combination thereof. In some embodiments, expressing more than one heterologous gene encoding at least ten enzymes selected from a steroidal alkaloid, steroidal saponin, or triterpenoid saponin biosynthetic pathway alters the content of the steroidal alkaloid, steroidal saponin, or triterpenoid saponin produced by that pathway or alters the content of at least one biosynthetic intermediate thereof, or a combination thereof.

In some embodiments, disclosed herein is a genetically modified cell having increased expression of at least one heterologous gene compared to a corresponding unmodified cell, said at least one heterologous gene encoding a cellulose synthase like G (CSLG) enzyme, wherein said genetically modified cell comprises an increased content of at least one steroidal alkaloid, steroidal saponin, or triterpenoid saponin, a derivative thereof, a metabolite thereof, or a biosynthetic intermediate thereof, compared to a corresponding unmodified cell. In some embodiments, the genetically modified cell further expresses at least one additional heterologous gene encoding an enzyme, said enzyme selected from the group consisting of a saponin beta-amyrin synthase, a cytochrome P450, a glycosyltransferase, an acyltransferase, and an UDP-glucose 6-dehydrogenase 1, or any combination thereof.

In some embodiments, derivatives of steroidal alkaloids, steroidal saponins, or triterpenoid saponins comprise glycosylated derivatives of, respectively, steroidal alkaloids, steroidal saponins, or triterpenoid saponins.

In some embodiments, a genetically modified cell expresses a heterologous CSLG and a heterologous saponin beta-amyrin synthase. In some embodiments, a genetically modified cell expresses a heterologous CSLG and at least one heterologous cytochrome P450. In some embodiments, a genetically modified cell expresses a heterologous CSLG and 1, 2, or 3 heterologous cytochrome P450s. In some embodiments, a genetically modified cell expresses a heterologous CSLG, a heterologous saponin beta-amyrin synthase, and 1, 2, or 3 heterologous cytochrome P450s. In some embodiments, a genetically modified cell expresses a heterologous CSLG and at least one heterologous glycosyl transferase. In some embodiments, a genetically modified cell expresses a heterologous CSLG and at least 1, 2, 3, or 4 heterologous glycosyl transferases. In some embodiments, a genetically modified cell expresses a heterologous CSLG, a heterologous saponin beta-amyrin synthase, and at least 1, 2, 3, or 4 heterologous glycosyl transferases. In some embodiments, a genetically modified cell expresses a heterologous CSLG, a heterologous saponin beta-amyrin synthase, at least 1, 2, or 3 heterologous cytochrome P450s, and at least 1, 2, 3, or 4 heterologous glycosyl transferases.

In certain instances, it may be necessary to express additional genes not specifically part of the steroidal alkaloid, steroidal saponin, or triterpenoid saponin pathway, homologous or heterologous, in order that necessary substrates are available. In some embodiments, a genetically modified cell expresses a heterologous gene encoding an enzyme necessary for production of substrates of a steroidal alkaloid, steroidal saponin, or triterpenoid saponin biosynthetic pathway.

In some embodiments, a heterologous gene encoding an enzyme selected from a saponin beta-amyrin synthase, a cytochrome P450, a C-2hydroxylase, a C-23 oxidase, a glycosyltransferase, a UDP-glucose 6-dehydrogenase 1, an acyltransferase, or a cellulose like synthase G (CSLG) is expressed in a genetically modified cell. In some embodiments, a heterologous gene encoding an enzyme selected from a saponin beta-amyrin synthase, a cytochrome P450, C-2hydroxylase, a C-23 oxidase, a glycosyltransferase, an acyltransferase, or a cellulose like synthase G (CSLG) is expressed in a genetically modified cell. In some embodiments, a heterologous gene encoding an enzyme selected from a saponin beta-amyrin synthase, a cytochrome P450, a glycosyltransferase, a UDP-glycosyltransferase, a fucosyltransferase, a xylosyltransferase, an acyltransferase, a BAHD-acyltransferase, or a cellulose like synthase G (CSLG) in expressed in a genetically modified cell.

In some embodiments, a genetically modified cell expresses at least one heterologous gene encoding an enzyme comprising a CSLG activity. In some embodiments, a genetically modified cell expresses at least one heterologous gene encoding an enzyme comprising a saponin beta-amyrin synthase activity. In some embodiments, a genetically modified cell expresses at least one heterologous gene encoding an enzyme comprising a cytochrome P450 activity. In some embodiments, a genetically modified cell expresses at least two heterologous gene encoding two enzymes comprising cytochrome P450 activity. In some embodiments, a genetically modified cell expresses at least three heterologous gene encoding three enzymes comprising cytochrome P450 activity. In some embodiments, an enzyme comprising cytochrome p450 activity comprises a C-2 hydroxylase activity. In some embodiments, an enzyme comprising cytochrome p450 activity comprises a C-23 oxidase activity.

In some embodiments, a genetically modified cell expresses heterologous genes encoding enzymes comprising a saponin beta-amyrin synthase activity, cytochrome P450 activities, and CSLG activity, wherein the cytochrome P450 enzymes may comprise an enzyme providing a C-2 hydroxylase activity, an enzyme providing a C-23 oxidase activity, a combination of enzymes providing cytochrome P450 activity, C-2 hydroxylase activity, and C-23 oxidase activity.

In some embodiments, a genetically modified cell expresses at least one heterologous gene encoding a glycosyltransferase enzyme activity. In some embodiments, the glycosyltransferase enzyme comprises a UDP-glycosyltransferase, a fucosyltransferase, or a xylosyltransferase. In some embodiments, a genetically modified cell expresses multiple heterologous genes encoding glycosyltransferase enzymes. In some embodiments, a genetically modified cell expresses multiple heterologous genes encoding glycosyltransferases, selected from a UDP-glycosyltransferase, a fucosyltransferase, or a xylosyltransferase, or a combination thereof. In some embodiments, a genetically modified cell expresses multiple heterologous genes encoding glycosyltransferases, selected from a combination of known glycosyltransferases known in the art. In some embodiments, a genetically modified cell expresses a heterologous gene encoding a CSLG activity and a heterologous gene encoding at least one glycosyltransferase enzyme activity. In some embodiments, a genetically modified cell expresses a heterologous gene encoding a CSLG activity and a heterologous gene encoding at least one glycosyltransferase enzyme activity selected from a UDP-glycosyltransferase, a fucosyltransferase, or a xylosyltransferase, or a combination thereof. In some embodiments, a genetically modified cell expresses a heterologous gene encoding a CSLG activity and a heterologous gene encoding at least one glycosyltransferase enzyme activity selected from a UDP-glycosyltransferase, a fucosyltransferase, or a xylosyltransferase, or any glycosyltransferase known in the art, or a combination thereof.

In some embodiments, a genetically modified cell expresses a heterologous gene encoding an acyltransferase. In some embodiments, a genetically modified cell expresses a heterologous gene encoding a benzylalcohol acetyl-, anthocyanin-O-hydroxy-cinnamoyl-, anthranilate-N-hydroxy-cinnamoyl/benzoyl-, deacetylvindoline acetyltransferase (BAHD) acyltransferase. In some embodiments, a genetically modified cell expresses a heterologous gene encoding an enzyme providing a CSLG activity and a heterologous gene encoding an enzyme encoding an acetyltransferase activity. In some embodiments, a genetically modified cell expresses a heterologous gene encoding an enzyme providing a CSLG activity and a heterologous gene encoding an enzyme encoding a BAHD acetyltransferase activity.

In some embodiments, a genetically modified cell expresses a heterologous gene encoding the encodes an enzyme activity for a triterpenoid saponin substrate, wherein the enzyme comprises a glycosyltransferase. In some embodiments, a genetically modified cell expresses a heterologous gene encoding the encodes an enzyme activity for a triterpenoid saponin substrate, wherein the enzyme comprises a UDP-glucose 6-dehydrogenase 1. In some embodiments, for example when the cell is a bacterial cell, a heterologous gene encodes a Squalene epoxidase, or a Cytochrome P450 reductase, or a UDP-glucose 6-dehydrogenase 1, or any combination thereof.

In some embodiments, disclosed herein is a genetically modified plant comprising an at least one cell having altered expression of at least a CSLG gene compared to the expression of CSLG in a corresponding unmodified cell, wherein the at least one cell comprises a heterologous CSLG gene, and said altered expression comprises a de novo expression of said heterologous gene. In some embodiments, disclosed herein is a genetically modified plant comprising an at least one cell having altered expression of at least a CSLG gene compared to the expression of CSLG in a corresponding unmodified cell, wherein the at least one cell comprises a heterologous CSLG gene, and said altered expression comprises expression of a heterologous gene in the presence of an endogenous gene, which may be functional or non-functional.

In some embodiments, the expression of a heterologous CSLG gene is a genetically modified plant comprises increased expression compared to a corresponding unmodified cell, and wherein said cell in said plant comprises altered content of at least a triterpenoid saponin, metabolite thereof, derivative thereof, or biosynthetic intermediate thereof, compared to the corresponding unmodified plant.

In some embodiments, disclosed herein is an array of enzymes that participate in the biosynthetic pathway of triterpenoid saponins, wherein the biosynthesis pathway includes production of saponin aglycones and glycosylated saponins, and glycosylated triterpenoid saponins. In some embodiments, disclosed herein is an array of genes encoding that enzymes that participate in the biosynthetic pathway of triterpenoid saponins, wherein the biosynthesis pathway includes production of saponin aglycones and glycosylated saponins, and glycosylated triterpenoid saponins, including but not limited to 3-O-[β-D-xylopyranosyl-(1->3)-β-D-glucuronopyranosyl]-28-O-[β-D-glucopyranosyl-(1->4)-a-L-rhamnopyranosyl-(1->2)-4-acetyl-β-D-fucopyranosyl]-medicagenic acid (Yossoside V; Compound 11); 3-O-[β-D-xylopyranosyl-(1->3)-β-D-glucuronopyranosyl]-28-O-[β-D-glucopyranosyl-(1->4)-α-L-rhamnopyranosyl-(1->2)-β-D-fucopyranosyl]-medicagenic acid (Yossoside IV; Compound 10); 3-O-[β-D-glucuronopyranosyl]-28-O-[β-D-glucopyranosyl-(1->4)-a-L-rhamnopyranosyl-(1->2)-β-D-fucopyranosyl]-medicagenic acid (Yossoside III; Compound 9); 3-O-[β-D-glucuronopyranosyl]-28-O-[α-L-rhamnopyranosyl-(1->2)-β-D-fucopyranosyl]-medicagenic acid (Yossoside II; Compound 8); and 3-O-β-D-glucuronopyranosyl-28-O-β-D-fucopyranosyl-medicagenic acid (Yossoside I; Compound 7) (see FIG. 20A).

For example, but not limited to the enzymes active in the spinach triterpenoid biosynthetic pathway of Compound 11, which are described in detail in the Examples. Table 16 provided in Example 20, provides the amino acid and nucleic acid sequences of the enzymes in the spinach biosynthetic pathway, wherein CSLG enzyme adds a glucuronic acid to medicagenic acid to make the triterpenoid saponin medicagenic acid 3-glucuronide. Table 19 disclosed in Example 24 provides the amino acid sequence of a CSLG isolated from Arabidopsis. Table 17 disclosed in the Examples provides the nucleic acid and amino acid sequences of CSLG homologs from other plant species.

In some embodiments, a genetically modified cell disclosed herein expresses at least one heterologous gene encoding an enzyme comprised in a steroidal alkaloid, steroidal saponin, or triterpenoid biosynthetic pathway. In some embodiments, a genetically modified cell disclosed herein expresses an array of heterologous gene encoding enzymes comprised in a steroidal alkaloid, steroidal saponin, or triterpenoid biosynthetic pathway. In some embodiments, a genetically modified cell disclosed herein expresses at least two heterologous gene encoding two enzymes comprised in a steroidal alkaloid, steroidal saponin, or triterpenoid biosynthetic pathway. In some embodiments, a genetically modified cell disclosed herein expresses at least three heterologous gene encoding three enzymes comprised in a steroidal alkaloid, steroidal saponin, or triterpenoid biosynthetic pathway. In some embodiments, a genetically modified cell disclosed herein expresses at least four heterologous gene encoding four enzymes comprised in a steroidal alkaloid, steroidal saponin, or triterpenoid biosynthetic pathway. In some embodiments, a genetically modified cell disclosed herein expresses at least 5 heterologous gene encoding five enzymes comprised in a steroidal alkaloid, steroidal saponin, or triterpenoid biosynthetic pathway. In some embodiments, a genetically modified cell disclosed herein expresses at least six heterologous gene encoding six enzymes comprised in a steroidal alkaloid, steroidal saponin, or triterpenoid biosynthetic pathway. In some embodiments, a genetically modified cell disclosed herein expresses at least seven heterologous gene encoding seven enzymes comprised in a steroidal alkaloid, steroidal saponin, or triterpenoid biosynthetic pathway. In some embodiments, a genetically modified cell disclosed herein expresses at least eight heterologous gene encoding eight enzymes comprised in a steroidal alkaloid, steroidal saponin, or triterpenoid biosynthetic pathway. In some embodiments, a genetically modified cell disclosed herein expresses at least nine heterologous gene encoding nine enzymes comprised in a steroidal alkaloid, steroidal saponin, or triterpenoid biosynthetic pathway. In some embodiments, a genetically modified cell disclosed herein expresses at least ten heterologous gene encoding ten enzymes comprised in a steroidal alkaloid, steroidal saponin, or triterpenoid biosynthetic pathway.

In some embodiments, disclosed herein is a genetically modified plant comprising at least one cell having altered expression of at least a cellulose synthase like G (CSLG) gene compared to the expression of CSLG in a corresponding unmodified plant, and wherein the genetically modified plant has an altered content of at least one steroidal alkaloid, a derivative thereof, a metabolite thereof, or a biosynthetic intermediate thereof, of at least one steroidal saponin, a derivative thereof, a metabolite thereof, or a biosynthetic intermediate thereof, or of at least one triterpenoid saponin, a derivative thereof, a metabolite thereof, or a biosynthetic intermediate thereof, compared to the corresponding unmodified plant. In some embodiments, the CSLG gene comprises the endogenous CSLG gene. In some embodiments, the CSLG gene comprises a heterologous CSLG gene.

In some embodiments, derivatives of steroidal alkaloids, steroidal saponins, or triterpenoid saponins comprise glycosylated derivatives of, respectively, steroidal alkaloids, steroidal saponins, or triterpenoid saponins.

In some embodiments, a genetically modified plant or plant part disclosed herein expresses at least one heterologous gene encoding an enzyme comprised in a steroidal alkaloid, steroidal saponin, or triterpenoid biosynthetic pathway. In some embodiments, a genetically modified plant or plant part disclosed herein expresses an array of heterologous gene encoding enzymes comprised in a steroidal alkaloid, steroidal saponin, or triterpenoid biosynthetic pathway. In some embodiments, a genetically modified plant or plant part disclosed herein expresses at least two heterologous gene encoding two enzymes comprised in a steroidal alkaloid, steroidal saponin, or triterpenoid biosynthetic pathway. In some embodiments, a genetically modified plant or plant part disclosed herein expresses at least three heterologous gene encoding three enzymes comprised in a steroidal alkaloid, steroidal saponin, or triterpenoid biosynthetic pathway. In some embodiments, a genetically modified plant or plant part disclosed herein expresses at least four heterologous gene encoding four enzymes comprised in a steroidal alkaloid, steroidal saponin, or triterpenoid biosynthetic pathway. In some embodiments, a genetically modified plant or plant part disclosed herein expresses at least 5 heterologous gene encoding five enzymes comprised in a steroidal alkaloid, steroidal saponin, or triterpenoid biosynthetic pathway. In some embodiments, a genetically modified plant or plant part disclosed herein expresses at least six heterologous gene encoding six enzymes comprised in a steroidal alkaloid, steroidal saponin, or triterpenoid biosynthetic pathway. In some embodiments, a genetically modified plant or plant part disclosed herein expresses at least seven heterologous gene encoding seven enzymes comprised in a steroidal alkaloid, steroidal saponin, or triterpenoid biosynthetic pathway. In some embodiments, a genetically modified plant or plant part disclosed herein expresses at least eight heterologous gene encoding eight enzymes comprised in a steroidal alkaloid, steroidal saponin, or triterpenoid biosynthetic pathway. In some embodiments, a genetically modified plant or plant part disclosed herein expresses at least nine heterologous gene encoding nine enzymes comprised in a steroidal alkaloid, steroidal saponin, or triterpenoid biosynthetic pathway. In some embodiments, a genetically modified plant or plant part disclosed herein expresses at least ten heterologous gene encoding ten enzymes comprised in a steroidal alkaloid, steroidal saponin, or triterpenoid biosynthetic pathway.

In some embodiments, heterologous genes encoding enzymes of the steroidal alkaloid, steroidal saponin, or triterpenoid biosynthetic pathway comprising enzymes selected from a beta-amyrin synthase, a cytochrome P450, multiple cytochrome P450s, a glycosyltransferase, multiple glycosyltransferases, an acyltransferase, or a CSLG, or any combination thereof. In some embodiments, heterologous genes encoding enzymes of the steroidal alkaloid, steroidal saponin, or triterpenoid biosynthetic pathway comprising enzyme selected from a beta-amyrin synthase, a cytochrome P450, multiple cytochrome P450s, a glycosyltransferase, multiple glycosyltransferases, an acyltransferase, or a CSLG, or any combination thereof, and in addition express at least one heterologous gene necessary to provide a biosynthetic pathway substrate or substrates. In some embodiments, heterologous genes encode enzymes of the steroidal alkaloid, steroidal saponin, or triterpenoid biosynthetic pathway comprising a beta-amyrin synthase, a cytochrome P450, multiple cytochrome P450s, a glycosyltransferase, multiple glycosyltransferases, an acyltransferase, or a CSLG, or any combination thereof, and in addition express a heterologous gene necessary to provide biosynthetic pathway substrates, for example but not limited to a UDP-glucose 6-dehydrogenase 1 enzyme.

In some embodiments, a genetically modified cell expresses at least one heterologous gene encoding an enzyme of the steroidal alkaloid/steroidal saponin/triterpenoid saponin biosynthetic pathway, said at least one heterologous gene encoding a CSLG enzyme.

In some embodiments, the at least one heterologous gene encodes a cytochrome P450 wherein said cytochrome P450 comprises a C2-hydroxylase or a C-23 oxidase, or a glycosyltransferase wherein said glycosyltransferase comprises a fructosyltransferase, a xylosyltransferase, or a UDP-glycosyltransferase, or a acyltransferase wherein said acyltransferase comprises a benzylalcohol acetyl-, anthocyanin-O-hydroxy-cinnamoyl-, anthranilate-N-hydroxy-cinnamyol/benzoyl-, deacetylvindoline (BAHD) acetyletransferase, or any combination thereof.

In some embodiments, expression of a heterologous gene or genes comprises increased expression compared to a corresponding unmodified cell. In some embodiments, increased expression of a gene results in increased content of an enzyme encoded by the gene, compared to the content of the enzyme in a corresponding unmodified cell. One skilled in the art would appreciate that increased expression may, in some embodiments, comprise overexpression of the gene or genes, with resultant increased presence of an enzyme of enzymes encoded by the gene or genes, compared with a corresponding unmodified cell. In some embodiments, increased expression may comprise expression of a gene or genes, and the production of a resultant enzyme activity or activities, not naturally found in the cell or organism, for example but not limited to a plant cell, a plant, a yeast cell, an algal cell, an insect cell, or a bacterial cell.

A skilled artisan would appreciate that a corresponding unmodified cell may in certain embodiments provide a control cell, wherein the heterologous gene is not expressed or is not overexpressed.

In some embodiments, expression of a gene or genes in a heterologous cell comprises transient expression of the gene or genes. In some embodiments, expression of a gene or genes in a heterologous cell comprises constitutive expression of the gene or genes. In some embodiments, expression of a gene or genes in a heterologous cell comprises transient expression, or constitutive expression, or a combination thereof, of the gene or genes.

A skilled artisan would appreciate that transient expression encompasses the temporary expression of a gene or genes that are expressed for a short time after a nucleic acid has been introduced into eukaryotic cells, for example a plant cell or a yeast cell. Conversely, constitutive expression, also known as stable expression, encompasses continuous expression of a gene or genes.

One of ordinary skill in the art would appreciate that the term “gene” may encompass a nucleic acid (e.g., DNA or RNA) sequence that comprises coding sequences necessary for the production of RNA or a polypeptide. A polypeptide can be encoded by a full-length coding sequence or by any part thereof. The term “parts thereof” when used in reference to a gene refers to fragments of that gene. The fragments may range in size from a few nucleotides to the entire gene sequence minus one nucleotide. Thus, “a nucleic acid sequence comprising at least a part of a gene” may comprise fragments of the gene or the entire gene.

The skilled artisan would appreciate that the term “gene” optionally also encompasses the coding regions of a structural gene and includes sequences located adjacent to the coding region on both the 5′ and 3′ ends for a distance of about 1 kb on either end such that the gene corresponds to the length of the full-length mRNA. The sequences which are located 5′ of the coding regions and which are present on the mRNA are referred to as 5′ non-translated sequences. The sequences which are located 3′ or downstream of the coding region and which are present on the mRNA are referred to as 3′ non-translated sequences.

In some embodiments, a gene comprises DNA sequence comprising the coding region. In some embodiments, a gene comprises DNA sequence comprising upstream and downstream regions, as well as the coding region, which comprises exons and any intervening introns of the gene. In some embodiments, upstream and downstream regions comprise non-coding regulatory regions. In some embodiments, upstream and downstream regions comprise regulatory sequences, for example but not limited to promoters, enhancers, and silencers. Non-limiting examples of regulatory sequences include, but are not limited to, AGGA box, TATA box, Inr, DPE, ZmUbi1, PvUbi1, PvUbi2, CaMV, 35S, OsAct1, zE19, E8, TA29, A9, pDJ3S, B33, PAT1, alcA, G-box, ABRE, DRE, and PCNA.

Regulatory regions, may in some embodiments, increase or decrease the expression of specific genes within a cell, for example but not limited to a yeast, an algal, an insect, a bacterium, or a plant cell or a plant described herein. Regulatory regions, may in some embodiments, increase or decrease the expression of specific genes within a plant cell. Regulatory regions, may in some embodiments, increase or decrease the expression of specific genes within an algal cell. Regulatory regions, may in some embodiments, increase or decrease the expression of specific genes within a yeast cell. Regulatory regions, may in some embodiments, increase or decrease the expression of specific genes within an insect cell. Regulatory regions, may in some embodiments, increase or decrease the expression of specific genes within a bacterium.

In some embodiments, a gene comprises the coding regions of the gene, which comprises exons and any intervening introns of the gene. In some embodiments, a gene comprises its regulatory sequences. In some embodiments, a gene comprises the gene promoter. In some embodiments, a gene comprises its enhancer regions. In some embodiments, a gene comprises 5′ non-coding sequences. In some embodiments, a gene comprises 3′ non-coding sequences.

In some embodiments, the skilled artisan would appreciate that DNA comprises a gene, which may include upstream and downstream sequences, as well as the coding region of the gene. In other embodiments, DNA comprises a cDNA (complementary DNA). One of ordinary skill in the art would appreciate that cDNA may encompass synthetic DNA reverse transcribed from RNA through the action of a reverse transcriptase. The cDNA may be single stranded or double stranded and can include strands that have either or both of a sequence that is substantially identical to a part of the RNA sequence or a complement to a part of the RNA sequence. Further, cDNA may include upstream and downstream regulatory sequences. In still other embodiments, DNA comprises a CDS (complete coding sequence). One of ordinary skill in the art would appreciate that CDS may encompass a DNA sequence, which encodes a full-length protein or polypeptide. A CDS typically begins with a start codon (“ATG”) and ends at (or one before) the first in-frame stop codon (“TAA”, “TAG”, or “TGA”). The skilled artisan would recognize that a cDNA, in one embodiment, comprises a CDS.

The terms “polynucleotide”, “polynucleotide sequence”, “nucleic acid sequence”, and “isolated polynucleotide” are used interchangeably herein. These terms encompass nucleotide sequences and the like. A polynucleotide may be a polymer of RNA or DNA or hybrid thereof, that is single- or double-stranded, linear or branched, and that optionally contains synthetic, non-natural or altered nucleotide bases. The terms also encompass RNA/DNA hybrids.

The terms “complementary” or “complement thereof” are used herein to refer to the sequences of polynucleotides which is capable of forming Watson & Crick base pairing with another specified polynucleotide throughout the entirety of the complementary region. This term is applied to pairs of polynucleotides based solely upon their sequences and not any particular set of conditions under which the two polynucleotides would actually bind.

The term “construct” as used herein refers to an artificially assembled or isolated nucleic acid molecule which includes the polynucleotide of interest. In general, a construct may include the polynucleotide or polynucleotides of interest, a marker gene, which in some cases can also be a gene of interest and appropriate regulatory sequences. It should be appreciated that the inclusion of regulatory sequences in a construct is optional, for example, such sequences may not be required in situations where the regulatory sequences of a host cell are to be used. Regulatory elements include, but are not limited to, a promoter, an enhance, an origin of replication, a transcription termination sequence, a polyadenylation signal, and the like. The term “construct” includes vectors but should not be seen as being limited thereto.

The term “operably linked” refers to the association of nucleic acid sequences on a single nucleic acid fragment so that the function of one is regulated by the other. For example, a promoter is operably linked with a coding sequence when it is capable of regulating the expression of that coding sequence (i.e., that the coding sequence is under the transcriptional control of the promoter). Coding sequences can be operably linked to regulatory sequences in a sense or antisense orientation. In some embodiments, the terms “operably linked” and “functionally linked” may be used interchangeably having all the same meanings and qualities.

The terms “promoter element,” “promoter,” or “promoter sequence” as used herein, refer to a DNA sequence that is generally located at the 5′ end (i.e. precedes) the coding region of a DNA polymer. The location of most promoters known in nature precedes the transcribed region. The promoter functions as a switch, activating the expression of a gene. If the gene is activated, it is said to be transcribed, or participating in transcription. Transcription involves the synthesis of mRNA from the gene. The promoter, therefore, serves as a transcriptional regulatory element and also provides a site for initiation of transcription of the gene into mRNA.

A polynucleotide sequence comprising a heterologous gene may be expressed in, for example, a plant cell, an algal cell, a yeast cell, an insect cell, or a bacterium under the control of a promoter that directs constitutive expression or regulated (transient) expression. In some embodiments, the plant cell is part of a whole plant or a portion thereof. Regulated expression comprises temporally or spatially regulated expression and any other form of inducible or repressible expression. Temporally means that the expression is induced at a certain time point, for instance, when a certain growth rate of the plant cell culture is obtained (e.g., the promoter is induced only in the stationary phase or at a certain stage of development). Spatially means that the promoter is only active in specific organs, tissues, or cells (e.g., for plants only in roots, leaves, epidermis, guard cells or the like). Other examples of regulated expression comprise promoters whose activity is induced or repressed by adding chemical or physical stimuli to the plant cell or yeast cell or an algal cell or an insect cell or a bacterium. In some embodiments, a promoter is an inducible promoter. In some embodiments, a promoter is a constitutive promoter. In some embodiments regulated expression of a heterologous gene of the triterpenoid biosynthetic pathway, for example but not limited to CSLG, comprises inducing or repressing gene expression by adding chemical or physical stimuli to a plant cell. In some embodiments regulated expression of a heterologous gene of the triterpenoid biosynthetic pathway, for example but not limited to CSLG, comprises inducing or repressing gene expression by adding chemical or physical stimuli to a yeast cell. In some embodiments regulated expression of a heterologous gene of the triterpenoid biosynthetic pathway, for example but not limited to CSLG, comprises inducing or repressing gene expression by adding chemical or physical stimuli to an algal cell. In some embodiments regulated expression of a heterologous gene of the triterpenoid biosynthetic pathway, for example but not limited to CSLG, comprises inducing or repressing gene expression by adding chemical or physical stimuli to an insect cell. In some embodiments regulated expression of a heterologous gene of the triterpenoid biosynthetic pathway, for example but not limited to CSLG, comprises inducing or repressing gene expression by adding chemical or physical stimuli to a bacterium.

In some embodiments, the expression is under control of environmental, hormonal, chemical, and/or developmental signals. Such promoters for plant cells include promoters that are regulated by (1) heat, (2) light, (3) hormones, such as abscisic acid, and methyl jasmonate (4) wounding or (5) chemicals such as salicylic acid, chitosans or metals. A constitutive promoter directs expression in a wide range of cells under a wide range of conditions. Examples of constitutive plant promoters useful for expressing heterologous polypeptides in plant cells include, but are not limited to, the cauliflower mosaic virus (CaMV) 35S promoter, which confers constitutive, high-level expression in most plant tissues including monocots, the nopaline synthase promoter and the octopine synthase promoter. In some embodiments, a promoter is an inducible promoter. In some embodiments provided herein, inducible plant promoters, include but are not limited to GAL1, GAL10, PHO5 and CUP1. In some embodiments, a yeast promoter is constitutive promoter. In some embodiments provided herein, constitutive yeast promoters, include but are not limited to PGK1, TDHS, ADH1, CYC1, ACT1, TEF1. In some embodiments, an algal promoter is an inducible promoter. In some embodiments, provided herein algal promoters may be regulated by changes of light, pH, salinity, temperature, or nutrients. In some embodiments, an inducible algal promoter responds to nitrogen levels, nitrogen stress, or nitrogen starvation. In some embodiments, an algal promoter is a constitutive promoter. In some embodiments, an inducible promoter for use in insect cells responds to environmental or nutritional signals.

An expression cassette is usually provided in a DNA or RNA construct comprising the at least one heterologous gene that is typically called an “expression vector,” which is any genetic element, e.g., a plasmid, a chromosome, a virus, behaving either as an autonomous unit of polynucleotide replication within a cell (i.e., capable of replication under its own control) or being rendered capable of replication by insertion into a host cell chromosome, having attached to it another polynucleotide segment, so as to bring about the replication and/or expression of the attached segment. Suitable vectors include, but are not limited to, plasmids, bacteriophages, cosmids, plant viruses, and artificial chromosomes. The expression cassette may be provided in a DNA construct, which also has at least one replication system. In addition to the replication system, there will frequently be at least one marker present, which may be useful in one or more hosts, or different markers for individual hosts.

In another embodiment, selection genes provide selective markers that confer resistance to a selective agent, such as an antibiotic or herbicide. Potentially transformed cells are exposed to the selective agent. In the population of surviving cells will be those cells where, generally, the resistance-conferring gene has been integrated and expressed at sufficient levels to permit cell survival. Cells may be tested further to confirm stable integration of the exogenous DNA.

Useful selective marker genes include those conferring resistance to antibiotics such as kanamycin (nptII), hygromycin B (aph IV), nptII, hpt, aadA and gentamycin (aac3 and aacC4) or resistance to herbicides such as glufosinate (bar or pat) and glyphosate (EPSPS). In another embodiment, the selection gene is an antimetabolite. In one embodiment, the antimetabolite is dhf.

Screenable markers which provide an ability to visually identify transformants can also be employed, e.g., a gene expressing a colored or fluorescent protein such as a cat, lacZ, uidA, luciferase or green fluorescent protein (GFP) or a gene expressing a beta-glucuronidase or uidA gene (GUS) for which various chromogenic substrates are known. It is also contemplated that combinations of screenable and selectable markers will be useful for identification of transformed cells.

In one embodiment, the selection gene is a positive selectable marker gene that is conditional on non-toxic agents that may be substrates for growth or that induce growth and differentiation of the transformed tissues.

In one embodiment, plant cells that survive exposure to the selective agent, or cells that have been scored positive in a screening assay, may be cultured in regeneration media and allowed to mature into plants. Developing plantlets can be transferred to soil less plant growth mix, and hardened off, e.g., in an environmentally controlled chamber at about 85% relative humidity, 600 ppm CO2, and 25-250 microeinsteins m−2 s−1 of light, prior to transfer to a greenhouse or growth chamber for maturation. Plants are preferably matured either in a growth chamber or greenhouse. Plants are regenerated from about 6 weeks to 10 months after a transformant is identified, depending on the initial tissue. During regeneration, cells are grown to plants on solid media at about 19 to 28° C. After regenerating plants have reached the stage of shoot and root development, they may be transferred to a greenhouse for further growth and testing. Plants may be pollinated using conventional plant breeding methods known to those of skill in the art and seed produced.

Progeny may be recovered from transformed plants and tested for expression of the exogenous recombinant polynucleotide. Useful assays include, for example, “molecular biological” assays, such as Southern and Northern blotting and PCR; “biochemical” assays, such as detecting the presence of RNA, e.g. double stranded RNA, or a protein product, e.g., by immunological means (ELISAs and Western blots) or by enzymatic function; plant part assays, such as leaf or root assays; and also, by analyzing the phenotype of the whole regenerated plant.

One of skill in the art will be able to select an appropriate vector for introducing the encoding nucleic acid sequence in a relatively intact state. Thus, any vector which will produce a host cell, e.g., plant protoplast, carrying the introduced encoding nucleic acid should be sufficient. The selection of the vector, or whether to use a vector, is typically guided by the method of transformation selected.

For plant cells, markers may a) code for protection against a biocide, such as antibiotics, toxins, heavy metals, certain sugars or the like; b) provide complementation, by imparting prototrophy to an auxotrophic host; or c) provide a visible phenotype through the production of a novel compound in the plant. In some embodiments, genes that may be employed include neomycin phosphotransferase (NPTII), hygromycin phosphotransferase (HPT), chloramphenicol acetyltransferase (CAT), nitrilase, and the gentamicin resistance gene. For plant host selection, non-limiting examples of suitable markers are β-glucuronidase, providing indigo production, luciferase, providing visible light production, Green Fluorescent Protein and variants thereof, NPTII, providing kanamycin resistance or G418 resistance, HPT, providing hygromycin resistance, and the mutated aroA gene, providing glyphosate resistance.

For yeast cells, markers may comprise marker providing complementation, by imparting prototrophy to an auxotrophic host, for example but not limited to IS, LEU, TRP, and URA marker genes encoding the necessary complementary activity. In some embodiments, yeast markers provide resistance to a compound toxic to the yeast, for example but not limited to kanMX4—resistance to G418; natMX4—resistance to nourseothricin; hphMX4—resistance to hygromycin B; patMX3—resistance to glufosinate; and ZEO—resistance to zeocin.

Promoter activity encompasses the extent of transcription of a polynucleotide sequence, homologue, variant or fragment thereof that is operably linked to the promoter whose promoter activity is being measured. The promoter activity may be measured directly by measuring the amount of RNA transcript produced, for example, by Northern blot or indirectly by measuring the product coded for by the RNA transcript, such as when a reporter gene is linked to the promoter.

In some embodiments, an at least one heterologous gene encoding an enzyme, disclosed herein, is operably linked to a promoter. In some embodiments, operable linkage to the promoter results in transient expression. In some embodiments, operable linkage to the promoter results in constitutive expression. In some embodiments, one heterologous gene may be operably linked to a constitutive promoter while another heterologous gene is operably linked to a promoter providing transient expression. In some embodiments, a polynucleotide sequence comprises the at least one heterologous gene encoding an enzyme, wherein said polynucleotide is optionally comprised within a vector.

As used herein, the term an “enhancer” refers to a DNA sequence which can stimulate promoter activity and may be an innate element of the promoter or a heterologous element inserted to enhance the level or tissue-specificity of a promoter.

The term “expression”, as used herein, refers to the production of a functional end-product e.g., an mRNA or a protein. In some embodiments, a genetically modified cell expresses at least one heterologous gene, wherein the functional end product comprises an mRNA, which in turn is transcribed to produce a functional end product comprising a steroidal alkaloid, a steroidal saponin, or a triterpenoid saponin.

The term “genetically modified cell” refers to a cell genetically modified by man. In some embodiments, the genetic modification includes transforming a plant cell or a yeast cell or an algal cell or an insect cell or a bacterium with heterologous polynucleotide comprising a gene encoding an enzyme. In some embodiments, the genetic modification includes transforming a plant cell or a yeast cell with heterologous polynucleotide comprising a gene encoding an enzyme active in a triterpenoid saponin biosynthetic pathway. A “genetically modified cell” and a “corresponding unmodified cell” as used herein refer to a genetically modified cell and to a cell of the same type lacking said modification, respectively.

In some embodiments, the “genetically modified cell” and the “corresponding unmodified cell” are, respectively, a genetically modified plant cell and a corresponding unmodified plant cell of the same type. In other embodiments, the “genetically modified cell” and the “corresponding unmodified cell” are, respectively, a genetically modified yeast cell and a corresponding unmodified yeast cell of the same type.

The term “genetically modified plant” refers to a plant comprising at least one cell genetically modified by man. The genetic modification includes modification of an endogenous gene(s), for example by introducing mutation(s) deletions, insertions, transposable element(s) and the like into an endogenous polynucleotide or gene of interest. Additionally, or alternatively, the genetic modification includes transforming the plant cell with heterologous polynucleotide. A “genetically modified plant” and a “corresponding unmodified plant” as used herein refer to a plant comprising at least one genetically modified cell and to a plant of the same type lacking said modification, respectively.

In some embodiments, suspensions of genetically modified cells and tissue cultures derived from the genetically modified cells are also encompassed. The cell suspension or tissue culture can be used for the production of desired steroidal alkaloids metabolites thereof, derivatives thereof, or biosynthetic intermediates thereof, or a combination thereof, steroidal saponins, metabolites thereof, derivatives thereof, or biosynthetic intermediates thereof, or a combination thereof, or triterpenoid saponins, metabolites thereof, derivatives thereof, or biosynthetic intermediates thereof, or a combination thereof. In some embodiments, the genetically modified cell or tissue culture is used for regenerating a genetically modified plant having at least one cell having altered expression of a CSLG enzyme.

One of ordinary skill in the art would appreciate that some embodiments further encompass seeds of the genetically modified plant, wherein plants grown from said seeds have altered expression of at least one CSLG in at least one cell compared to plants grown from corresponding unmodified seeds or seeds from a corresponding unmodified plant, thereby having at least one cell having altered content of at least one steroidal alkaloid, steroidal saponin, or triterpenoid saponin.

If transformation techniques require use of tissue culture, transformed cells may be regenerated into plants in accordance with techniques well known to those of skill in the art. The regenerated plants may then be grown and crossed with the same or different plant varieties using traditional breeding techniques to produce seeds, which are then selected under the appropriate conditions.

One of ordinary skill in the art would appreciate that a genetically modified plant may encompass a plant comprising at least one cell genetically modified by man. In some embodiment, the genetic modification includes transforming at least one plant cell or yeast cell or algal cell or insect cell or bacterium with a heterologous polynucleotide comprising a heterologous gene described herein. In some embodiment, the genetic modification includes transforming at least one plant cell or yeast cell or algal cell or insect cell or bacterium with a heterologous polynucleotide comprising more than one heterologous gene, each encoding an enzyme. In some embodiment, the genetic modification includes transforming at least one plant cell or yeast cell or algal cell or insect cell or bacterium with multiple heterologous polynucleotides encoding multiple heterologous genes encoding enzymes.

The skilled artisan would appreciate that a genetically modified plant comprising transforming at least one plant cell with a heterologous polynucleotide or multiple heterologous polynucleotides may in certain embodiments be termed a “transgenic plant”.

A skilled artisan would appreciate that a comparison of a “genetically modified plant” to a “corresponding unmodified plant” as used herein encompasses comparing a plant comprising at least one genetically modified cell and to a plant of the same type lacking the modification.

The skilled artisan would appreciate that the term “transgenic” when used in reference to a plant as disclosed herein encompasses a plant that contains at least one heterologous transcribable polynucleotide in one or more of its cells. The skilled artisan would appreciate that the term “transgenic” when used in reference to a yeast as disclosed herein encompasses a yeast that contains at least one heterologous transcribable polynucleotide. The term “transgenic material” encompasses broadly a plant or a part thereof, including at least one cell, multiple cells or tissues that contain at least one heterologous polynucleotide in at least one of cell. Thus, comparison of a “transgenic plant” and a “corresponding non transgenic plant”, or of a “genetically modified plant comprising at least one cell having altered expression, wherein said plant comprising at least one cell comprising a heterologous transcribable polynucleotide” and a “corresponding un modified plant” encompasses comparison of the “transgenic plant” or “genetically modified plant” to a plant of the same type lacking said heterologous transcribable polynucleotide. A skilled artisan would appreciate that, in some embodiments, a “transcribable polynucleotide” comprises a polynucleotide that can be transcribed into an RNA molecule by an RNA polymerase.

The terms “transformants” or “transformed cells” include the primary transformed cell and cultures derived from that cell without regard to the number of transfers. All progeny may not be precisely identical in DNA content, due to deliberate or inadvertent mutations. Mutant progeny that have the same functionality as screened for in the originally transformed cell are included in the definition of transformants.

Transformation of a cell may be stable or transient. The term “transient transformation” or “transiently transformed” refers to the introduction of one or more exogenous polynucleotides into a cell in the absence of integration of the exogenous polynucleotide into the host cell's genome. In contrast, the term “stable transformation” or “stably transformed” refers to the introduction and integration of one or more exogenous polynucleotides into the genome of a cell. The term “stable transformant” refers to a cell which has stably integrated one or more exogenous polynucleotides into the genomic or organellar DNA. It is to be understood that an organism or its cell transformed with the nucleic acids, constructs and/or vectors disclosed herein can be transiently as well as stably transformed.

The skilled artisan would appreciate that the term “construct” may encompass an artificially assembled or isolated nucleic acid molecule which includes the polynucleotide of interest. In general, a construct may include the polynucleotide or polynucleotides of interest, a marker gene which in some cases can also be a gene of interest and appropriate regulatory sequences. It should be appreciated that the inclusion of regulatory sequences in a construct is optional, for example, such sequences may not be required in situations where the regulatory sequences of a host cell are to be used. The term construct includes vectors but should not be seen as being limited thereto.

Additionally, or alternatively, in some embodiments, the genetic modification includes transforming at least one plant cell with a heterologous polynucleotide or multiple heterologous polynucleotides. The skilled artisan would appreciate that a genetically modified plant comprising transforming at least one plant cell with a heterologous polynucleotide or multiple heterologous polynucleotides may in certain embodiments be termed a “transgenic plant”.

Viral vectors are useful for transformation of more transformation-resistant plants (e.g., soybean or common bean). In some embodiments, viral vectors, such as bean pod mottle virus (BPMV; genus Comovirus) vectors, are used for foreign gene expression and virus-induced gene silencing (VIGS) (Zhang et al. (May 2010) Plant Physiol. 153: 52-65 [“Zhang 2010” ])). Cells are transformed, e.g., via biolistics or via direct DNA-rubbing inoculation (Zhang 2010).

In one embodiment, a gene gun or a biolistic particle delivery system (biolistics) is used for plant transformation to deliver exogenous DNA (transgenes) to cells (Rech et al. (2008) Nature Protocols 3(3): 410-418 [“Rech 2008” ]). In some embodiments, the plasmid is designed and apical meristems of plants (e.g., soybean, bean, cotton) are bombarded with microparticle-coated DNA, followed by in vitro culture and selection of transgenic plants (Rech 2008). In other embodiments, a callus of undifferentiated plant cells or a group of immature embryos growing on gel medium in vitro. In some embodiments, the cells are then treated with a series of plant hormones, such as auxins or gibberellins to obtain plants. Steroidal alkaloids metabolites thereof, derivatives thereof, or biosynthetic intermediates thereof, or a combination thereof, steroidal saponins, metabolites thereof, derivatives thereof, or biosynthetic intermediates thereof, or a combination thereof, or triterpenoid saponins, metabolites thereof, derivatives thereof, or biosynthetic intermediates thereof, or a combination thereof, can be produced through such transgenic plants or plant cells.

“Transient expression” of the proteins may be achieved by various means known in the art. In one embodiment, transient expression of the proteins is achieved by the use of genetically modified viruses. In some embodiments, agroinfiltration is used to induce transient expression of genes in a plant or an isolated leaf or another portion of a plant. A suspension of Agrobacterium (e.g., Agrobacterium tumefaciens) is introduced into the plant by, e.g., direct injection or vacuum filtration, or is brought into association with plant cells immobilized on a porous support (plant cell packs). The bacteria transfer the desired gene into the plant cells via transfer of Ti plasmid-derived T-DNA.

In some embodiments, the method of transformation of algae comprises any of the methods as described hereinabove. In one embodiment, transformation of algae is accomplished using glass bead-assisted transformation, particle gun-mediated (biolistic) transformation, treatment with cellulolytic enzymes to weaken their cell walls, or homologous recombination.

In another aspect, the nucleic acids disclosed herein can be transformed into algae. In one embodiment, the alga is a single cell alga. In another embodiment, the alga is a multi-cellular alga. In one embodiment, the alga is a cyanobacterium, diatom, Chlamydomonas, Dunaliella, or Hematococus. The genes can be over expressed in algae. Steroidal alkaloids metabolites thereof, derivatives thereof, or biosynthetic intermediates thereof, or a combination thereof; steroidal saponins, metabolites thereof, derivatives thereof, or biosynthetic intermediates thereof, or a combination thereof, or triterpenoid saponins, metabolites thereof, derivatives thereof, or biosynthetic intermediates thereof, or a combination thereof, can be produced through such transgenic algae. Method for algae transformation are well known in the art and fully described in U.S. Patent Application Publications US 20150011008; US 20150004704; US 20130130389; US 20120094385; US 20120034698; US 20110300633; and US 20040133937, which are incorporated by reference herein in their entirety. Further, cultivation of microalgae is well known in the art and has been described in Vuppaladadiyam et al., (2018) “Microalgae cultivation and metabolites production: a comprehensive review.” Biofuls, Bioprod. Bioref 12:304-324, which is incorporated by reference herein in its entirety.

In another aspect, the nucleic acids disclosed herein can be transformed into yeast. The genes can be over expressed in yeast. Methods for yeast transformation are described herein below and are well known in the art and fully described in U.S. Patent Application Publications US 20090264320; US 20010031724; US 20030049785; US 20050158861; US 20070264716; US 20090325247; and US 20100190223, which are incorporated by reference herein in their entirety. Steroidal alkaloids metabolites thereof, derivatives thereof, or biosynthetic intermediates thereof, or a combination thereof, steroidal saponins, metabolites thereof, derivatives thereof, or biosynthetic intermediates thereof, or a combination thereof, or triterpenoid saponins, metabolites thereof, derivatives thereof, or biosynthetic intermediates thereof, or a combination thereof, can be produced through such transgenic yeast.

In another embodiment, the nucleic acids disclosed herein can be transformed into a virus. In another embodiment, the nucleic acids may be over-expressed in a virus. Steroidal alkaloids metabolites thereof, derivatives thereof, or biosynthetic intermediates thereof, or a combination thereof, steroidal saponins, metabolites thereof, derivatives thereof, or biosynthetic intermediates thereof, or a combination thereof, or triterpenoid saponins, metabolites thereof, derivatives thereof, or biosynthetic intermediates thereof, or a combination thereof, can be produced through such viral over expression.

In some embodiments, in bacterial systems, a number of expression vectors can be advantageously selected depending upon the use intended for the polypeptide expressed. In one embodiment, large quantities of polypeptide are desired. In one embodiment, vectors that direct the expression of high levels of the protein product, possibly as a fusion with a hydrophobic signal sequence, which directs the expressed product into the periplasm of the bacteria or the culture medium where the protein product is readily purified are desired. In one embodiment, certain fusion protein engineered with a specific cleavage site to aid in recovery of the polypeptide. In one embodiment, vectors adaptable to such manipulation include, but are not limited to, the pET series of Escherichia coli expression vectors (Studier et al., Methods in Enzymol. 185:60-89 1990).

In some embodiments, a polynucleotide sequence comprises the at least one heterologous gene. In some embodiments, the polynucleotide sequence encodes an enzyme. In some embodiments, the polynucleotide sequence encodes an enzyme of a steroidal alkaloid, steroidal saponin, or triterpenoid saponin biosynthetic pathway. In some embodiments, the polynucleotide sequence encodes an enzyme necessary for the production of a substrate or substrates of a steroidal alkaloid, steroidal saponin, or triterpenoid saponin biosynthetic pathway.

In some embodiments, a polynucleotide sequence comprises the at least 2, 3, 4, 5, 6, 7, 8, 9, or 10, or more heterologous genes. In some embodiments, the polynucleotide sequence encodes 2, 3, 4, 5, 6, 7, 8, 9, or 10, or more enzyme, wherein each gene encodes a single enzyme. In some embodiments, the polynucleotide sequence encodes 2, 3, 4, 5, 6, 7, 8, 9, or 10 enzymes of a steroidal alkaloid, steroidal saponin, or triterpenoid saponin biosynthetic pathway. In some embodiments, the 7a polynucleotide sequence encodes 2, 3, 4, 5, 6, 7, 8, 9, or 10, or more enzymes of a steroidal alkaloid, steroidal saponin, or triterpenoid saponin pathway and an additional enzyme necessary for the production of a substrate of a steroidal alkaloid, steroidal saponin, or triterpenoid saponin biosynthetic pathway. In some embodiments, the polynucleotide sequence encodes 2, 3, 4, 5, 6, 7, 8, 9, or 10, or more enzymes of a steroidal alkaloid, steroidal saponin, or triterpenoid saponin pathway and additional enzymes necessary for the production of substrates of a steroidal alkaloid, steroidal saponin, or triterpenoid saponin biosynthetic pathway.

In some embodiments, the term “a nucleotide sequence” or “a polynucleotide sequence” encompasses a single contiguous polynucleotide sequence, and in other embodiments, the term encompasses multiple contiguous nucleotide sequences.

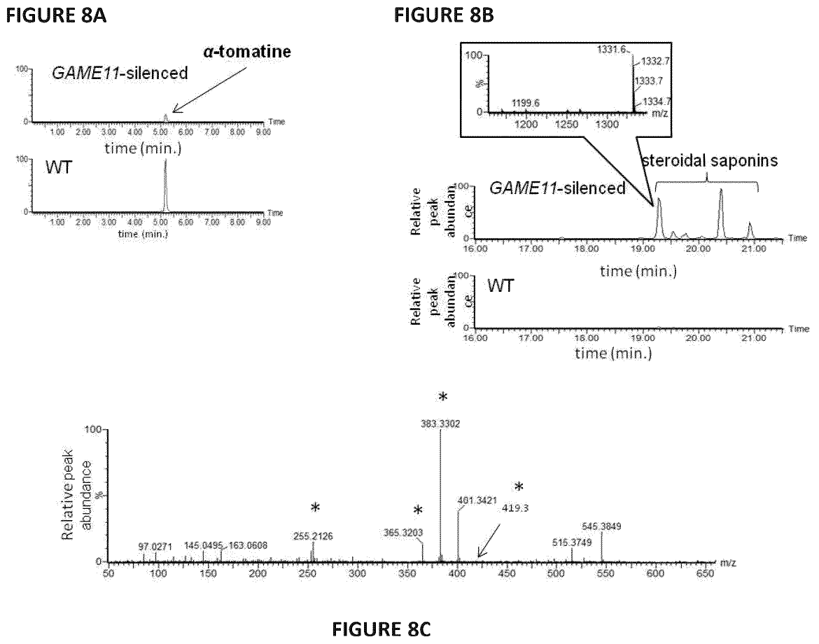

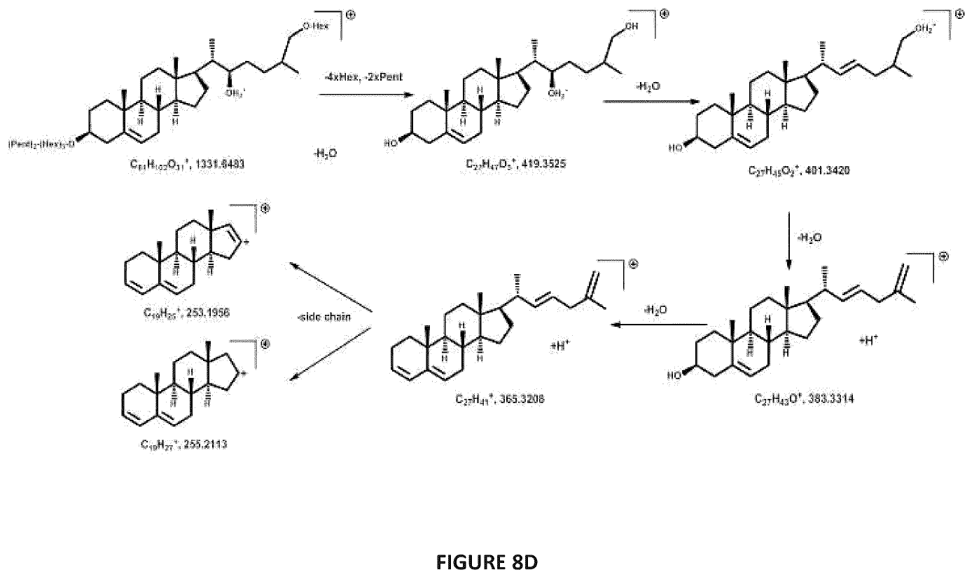

Based on the co-expressed gene array disclosed herein, a pathway from cholesterol to, e.g., α-tomatine is proposed (FIG. 1). It has been previously described that cholesterol is hydroxylated at C22 by GAME7 (US 2012/0159676) followed by GAME8 hydroxylation at the C26 position. The 22,26-dihydroxycholesterol is than hydroxylated at C16 and oxidized at C22 followed by closure of the E-ring by GAME11 and GAME6 to form the furostanol-type aglycone. This order of reactions is supported by the finding herein showing the accumulation of cholestanol-type saponins, lacking hydroxylation at C16 and the hemi-acetal E-ring when silencing GAME11 (FIGS. 8A-D). The furostanol-intermediate is oxidized by GAME4 to its 26-aldehyde which is the substrate for transamination catalyzed by GAME12. Nucleophilic attack of the amino-nitrogen at C22 leads to the formation of tomatidenol which is dehydrogenated to tomatidine. Tomatidine is subsequently converted by GAME1 to T-Gal (Itkin et al., 2011 supra). T-Gal in its turn is glucosylated by GAME17 into 7-tomatine, which is further glucosylated by GAME18 to P31-tomatine that is finally converted to a-tomatine by GAME2 (FIG. 1).

As described herein, by modifying expression of an enzyme and/or other protein involved in the biosynthetic pathway, the level of steroidal alkaloids, steroidal glycoalkaloids and steroidal saponin can be altered.

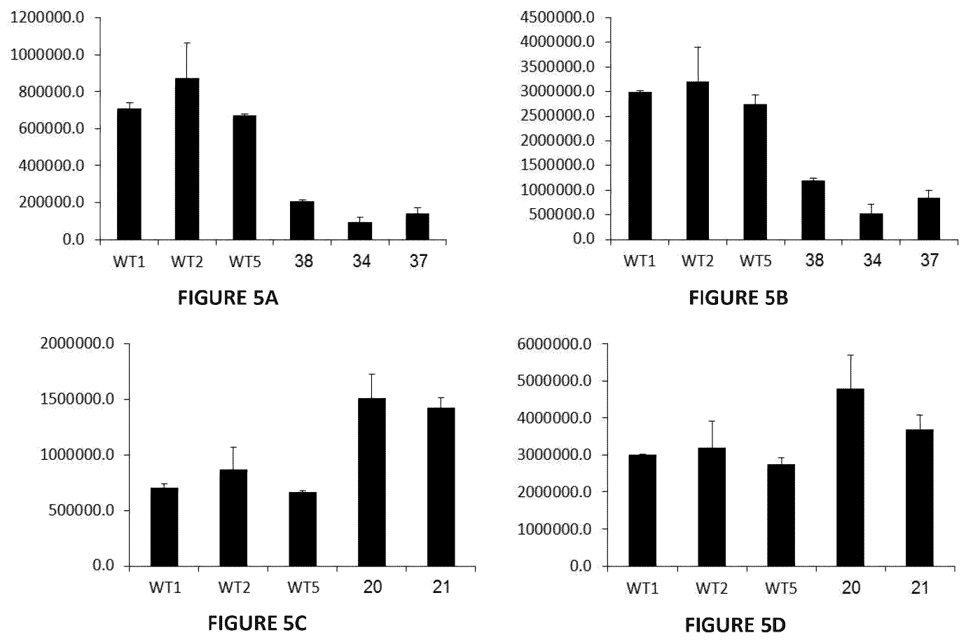

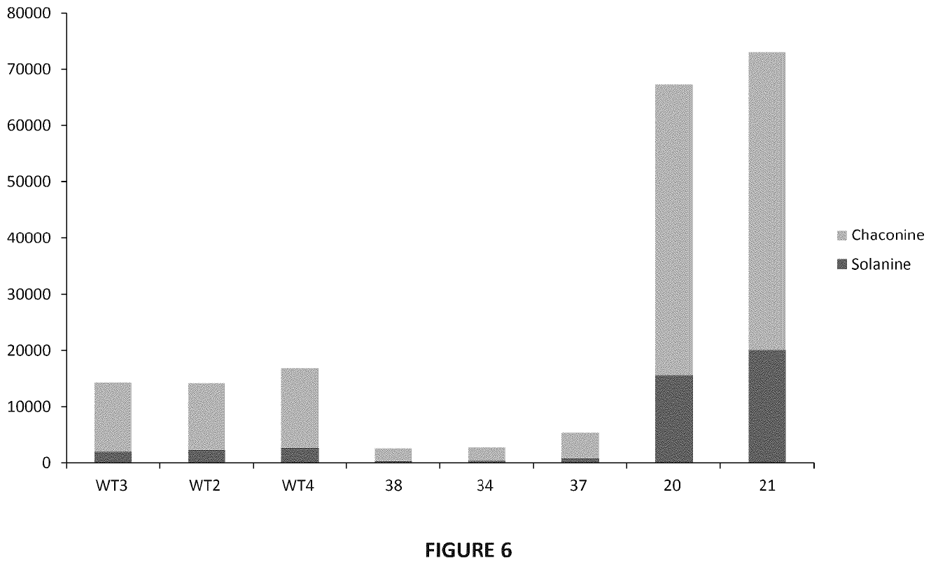

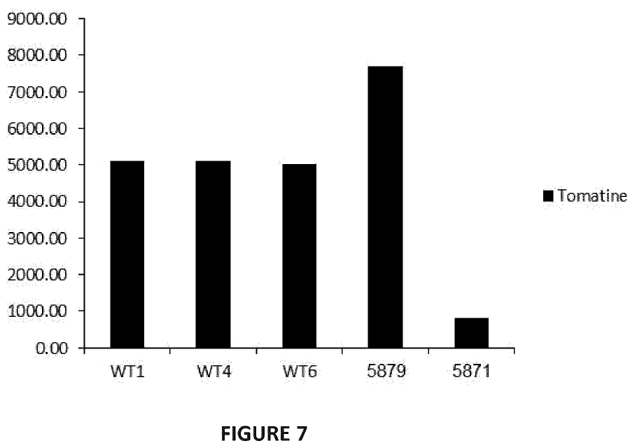

Silencing of a single gene co-expressed with the clustered enzyme-encoding gene in potato plant, resulted in significant reduction in the amount of the steroidal glycoalkaloids α-chaconine and a-solanine, while overexpression of this gene resulted in significant increase in the content of these substances (FIGS. 5 and 6). This gene was found to include coding sequence comprising an AP2 domain, and therefore postulated to be a transcription factor, designated herein GAME9-transcription factor, encoded by GAME9.

A genetically modified plant comprising at least one cell having altered expression of at least one gene selected from the group consisting of a gene encoding GAME9-transcription factor, a gene encoding 2-oxoglutarate-dependent dioxygenase, a gene encoding basic helix-loop-helix (BHLH)-transcription factor or a combination thereof, wherein the genetically modified plant has an altered content of at least one steroidal alkaloid or a derivative, metabolite, or biosynthetic intermediate thereof compared to a corresponding unmodified plant, has been produced. As exemplified herein for 2-oxoglutarate-dependent dioxygenase (GAME11), manipulating the expression of the genes of the present invention can further lead to the manipulation of steroidal saponin synthesis.

Thus, according to additional aspect, provided herein is a genetically modified organism comprising at least one cell having altered expression of at least one gene selected from the group consisting of a gene encoding GAME9-transcription factor, a gene encoding 2-oxoglutarate-dependent dioxygenase, a gene encoding basic helix-loop-helix (BHLH)-transcription factor or a combination thereof compared to an unmodified or unedited organism, wherein the genetically modified organism has an altered content of at least one compound selected from steroidal saponins and/or steroidal alkaloids and their glycosylated and other derivatives thereof, metabolites thereof, or biosynthetic intermediates thereof, compared to a corresponding unmodified or unedited organism.

Unexpectedly, the present invention now shows that SGA levels can be severely reduced in potato tubers by modifying expression of an enzyme and/or transcription factors involved in the steroidal alkaloid biosynthetic pathway.

According to certain embodiments, the expression of the at least one gene selected from the group consisting of a gene encoding GAME9-transcription factor, a gene encoding 2-oxoglutarate-dependent dioxygenase, a gene encoding BHLH-transcription factor or the combination thereof in the genetically modified plant is inhibited compared to its expression in the corresponding unmodified or unedited plant, thereby the genetically modified plant comprises reduced content of at least one steroidal alkaloid or a derivative, metabolite, or biosynthetic intermediate thereof, compared to a corresponding unmodified plant.

The genes and encoded enzymes thereof, active in the spinach triterpenoid biosynthetic pathway of Compound 11, are described in detail in the Examples. Table 17, provided in Example 20, provides the amino acid and nucleic acid sequences of these enzyme and the genes encoding them. Table 19 provided in Example 24 provides the amino acid sequence of an additional CSLG enzyme, isolated from Arabidopsis.

In some embodiments, the polynucleotide sequence of said at least one heterologous gene is set forth in any one of SEQ ID NOS: 125, 127, 30, 32, 34, 36, 38, 40, 45, 46, 47, 51, 53, 55, 57, 59, 61, 63, 65, 80, 93, 95, 97, 99, 101, 103, or 105. In some embodiments, the polynucleotide sequence of said at least one heterologous gene comprises a nucleic acid sequence having at least 55% identity to the nucleic acid sequence set forth in any one of SEQ ID NOS: 125, 127, 30, 32, 34, 36, 38, 40, 45, 46, 47, 51, 53, 55, 57, 59, 61, 63, 65, 80, 93, 95, 97, 99, 101, 103, or 105.

In some embodiments, the polynucleotide sequence of said at least one heterologous gene is set forth in any one of SEQ ID NOS: 125, 127, 30, 32, 34, 36, 38, 40, 45, 46, 47, 51, 53, 55, 57, 59, 61, 63, 65, 80, 93, 95, 97, 99, 101, 103, or 105. In some embodiments, the polynucleotide sequence of said at least one heterologous gene comprises a nucleic acid sequence having at least 80% identity to the nucleic acid sequence set forth in any one of SEQ ID NOS: 125, 127, 30, 32, 34, 36, 38, 40, 45, 46, 47, 51, 53, 55, 57, 59, 61, 63, 65, 80, 93, 95, 97, 99, 101, 103, or 105.

In some embodiments, the nucleic acid sequence encoding said at least one heterologous CSLG enzyme is set forth in any one of SEQ ID NOS: 125, 127, 30, 32, 34, 36, 38, 40, 65, 80, 93, 95, 97, 99, 101, 103, or 105. In some embodiments, the nucleic acid sequence encoding said at least one heterologous CSLG enzyme is set forth in a homolog thereof having at least 55% identity to and at least 55% coverage of, the nucleic acid sequence set forth in any one of SEQ ID NOS: 125, 127, 30, 32, 34, 36, 38, 40, 65, 80, 93, 95, 97, 99, 101, 103, or 105. In some embodiments, the nucleic acid sequence encoding said at least one heterologous CSLG enzyme is set forth in any one of SEQ ID NOS: 125, 127, 30, 32, 34, 36, 38, 40, 65, 80, 93, 95, 97, 99, 101, 103, or 105. In some embodiments, the nucleic acid sequence encoding said at least one heterologous CSLG enzyme is set forth in a homolog thereof having at least 55% identity to and at least 80% coverage of, the nucleic acid sequence set forth in any one of SEQ ID NOS: 125, 127, 30, 32, 34, 36, 38, 40, 65, 80, 93, 95, 97, 99, 101, 103, or 105.

In some embodiments, the nucleic acid sequence encoding said at least one heterologous CSLG enzyme is set forth in any one of SEQ ID NOS: 125, 127, 30, 32, 34, 36, 38, 40, 65, 80, 93, 95, 97, 99, 101, 103, or 105. In some embodiments, the nucleic acid sequence encoding said at least one heterologous CSLG enzyme is set forth in a homolog thereof having at least 80% identity to and at least 80% coverage of, the nucleic acid sequence set forth in any one of SEQ ID NOS: 125, 127, 30, 32, 34, 36, 38, 40, 65, 80, 93, 95, 97, 99, 101, 103, or 105.

In some embodiments, the polynucleotide sequence of said at least one heterologous gene is set forth in SEQ ID NO: 125. In some embodiments, the polynucleotide sequence of said at least one heterologous gene is set forth in SEQ ID NO: 127. In some embodiments, the polynucleotide sequence of said at least one heterologous gene is set forth in SEQ ID NO: 30. In some embodiments, the polynucleotide sequence of said at least one heterologous gene is set forth in SEQ ID NO: 32. In some embodiments, the polynucleotide sequence of said at least one heterologous gene is set forth in SEQ ID NO: 34. In some embodiments, the polynucleotide sequence of said at least one heterologous gene is set forth in SEQ ID NO: 36. In some embodiments, the polynucleotide sequence of said at least one heterologous gene is set forth in SEQ ID NO: 38. In some embodiments, the polynucleotide sequence of said at least one heterologous gene is set forth in SEQ ID NO: 40. In some embodiments, the polynucleotide sequence of said at least one heterologous gene is set forth in SEQ ID NO: 45. In some embodiments, the polynucleotide sequence of said at least one heterologous gene is set forth in SEQ ID NO: 46. In some embodiments, the polynucleotide sequence of said at least one heterologous gene is set forth in SEQ ID NO: 47. In some embodiments, the polynucleotide sequence of said at least one heterologous gene is set forth in SEQ ID NO: 51. In some embodiments, the polynucleotide sequence of said at least one heterologous gene is set forth in SEQ ID NO: 53. In some embodiments, the polynucleotide sequence of said at least one heterologous gene is set forth in SEQ ID NO: 55. In some embodiments, the polynucleotide sequence of said at least one heterologous gene is set forth in SEQ ID NO: 57. In some embodiments, the polynucleotide sequence of said at least one heterologous gene is set forth in SEQ ID NO: 59. In some embodiments, the polynucleotide sequence of said at least one heterologous gene is set forth in SEQ ID NO: 61. In some embodiments, the polynucleotide sequence of said at least one heterologous gene is set forth in SEQ ID NO: 163 In some embodiments, the polynucleotide sequence of said at least one heterologous gene is set forth in SEQ ID NO: 65. In some embodiments, the polynucleotide sequence of said at least one heterologous gene is set forth in SEQ ID NO: 80. In some embodiments, the polynucleotide sequence of said at least one heterologous gene is set forth in SEQ ID NO: 93. In some embodiments, the polynucleotide sequence of said at least one heterologous gene is set forth in SEQ ID NO: 95. In some embodiments, the polynucleotide sequence of said at least one heterologous gene is set forth in SEQ ID NO: 97. In some embodiments, the polynucleotide sequence of said at least one heterologous gene is set forth in SEQ ID NO: 99. In some embodiments, the polynucleotide sequence of said at least one heterologous gene is set forth in SEQ ID NO: 101. In some embodiments, the polynucleotide sequence of said at least one heterologous gene is set forth in SEQ ID NO: 103. In some embodiments, the polynucleotide sequence of said at least one heterologous gene is set forth in SEQ ID NO: 105.

In some embodiments, the polynucleotide sequence of said at least one heterologous gene comprises a nucleic acid sequence having at least 55% identity to the nucleic acid sequence set forth in SEQ ID NO: 125. In some embodiments, the polynucleotide sequence of said at least one heterologous gene comprises a nucleic acid sequence having at least 55% identity to the nucleic acid sequence set forth in SEQ ID NO: 127. In some embodiments, the polynucleotide sequence of said at least one heterologous gene comprises a nucleic acid sequence having at least 55% identity to the nucleic acid sequence set forth in SEQ ID NO: 30. In some embodiments, the polynucleotide sequence of said at least one heterologous gene comprises a nucleic acid sequence having at least 55% identity to the nucleic acid sequence set forth in SEQ ID NO: 32. In some embodiments, the polynucleotide sequence of said at least one heterologous gene comprises a nucleic acid sequence having at least 55% identity to the nucleic acid sequence set forth in SEQ ID NO: 34. In some embodiments, the polynucleotide sequence of said at least one heterologous gene comprises a nucleic acid sequence having at least 55% identity to the nucleic acid sequence set forth in SEQ ID NO: 36. In some embodiments, the polynucleotide sequence of said at least one heterologous gene comprises a nucleic acid sequence having at least 55% identity to the nucleic acid sequence set forth in SEQ ID NO: 38. In some embodiments, the polynucleotide sequence of said at least one heterologous gene comprises a nucleic acid sequence having at least 55% identity to the nucleic acid sequence set forth in SEQ ID NO: 40. In some embodiments, the polynucleotide sequence of said at least one heterologous gene comprises a nucleic acid sequence having at least 55% identity to the nucleic acid sequence set forth in SEQ ID NO: 45. In some embodiments, the polynucleotide sequence of said at least one heterologous gene comprises a nucleic acid sequence having at least 55% identity to the nucleic acid sequence set forth in SEQ ID NO: 46. In some embodiments, the polynucleotide sequence of said at least one heterologous gene comprises a nucleic acid sequence having at least 55% identity to the nucleic acid sequence set forth in SEQ ID NO: 47. In some embodiments, the polynucleotide sequence of said at least one heterologous gene comprises a nucleic acid sequence having at least 55% identity to the nucleic acid sequence set forth in SEQ ID NO: 51. In some embodiments, the polynucleotide sequence of said at least one heterologous gene comprises a nucleic acid sequence having at least 55% identity to the nucleic acid sequence set forth in SEQ ID NO: 53. In some embodiments, the polynucleotide sequence of said at least one heterologous gene comprises a nucleic acid sequence having at least 55% identity to the nucleic acid sequence set forth in SEQ ID NO: 55. In some embodiments, the polynucleotide sequence of said at least one heterologous gene comprises a nucleic acid sequence having at least 55% identity to the nucleic acid sequence set forth in SEQ ID NO: 57. In some embodiments, the polynucleotide sequence of said at least one heterologous gene comprises a nucleic acid sequence having at least 55% identity to the nucleic acid sequence set forth in SEQ ID NO: 59. In some embodiments, the polynucleotide sequence of said at least one heterologous gene comprises a nucleic acid sequence having at least 55% identity to the nucleic acid sequence set forth in SEQ ID NO: 61. In some embodiments, the polynucleotide sequence of said at least one heterologous gene comprises a nucleic acid sequence having at least 55% identity to the nucleic acid sequence set forth in SEQ ID NO: 63. In some embodiments, the polynucleotide sequence of said at least one heterologous gene comprises a nucleic acid sequence having at least 55% identity to the nucleic acid sequence set forth in SEQ ID NO: 65. In some embodiments, the polynucleotide sequence of said at least one heterologous gene comprises a nucleic acid sequence having at least 55% identity to the nucleic acid sequence set forth in SEQ ID NO: 80. In some embodiments, the polynucleotide sequence of said at least one heterologous gene comprises a nucleic acid sequence having at least 55% identity to the nucleic acid sequence set forth in SEQ ID NO: 93. In some embodiments, the polynucleotide sequence of said at least one heterologous gene comprises a nucleic acid sequence having at least 55% identity to the nucleic acid sequence set forth in SEQ ID NO: 95. In some embodiments, the polynucleotide sequence of said at least one heterologous gene comprises a nucleic acid sequence having at least 55% identity to the nucleic acid sequence set forth in SEQ ID NO: 97. In some embodiments, the polynucleotide sequence of said at least one heterologous gene comprises a nucleic acid sequence having at least 55% identity to the nucleic acid sequence set forth in SEQ ID NO: 99. In some embodiments, the polynucleotide sequence of said at least one heterologous gene comprises a nucleic acid sequence having at least 55% identity to the nucleic acid sequence set forth in SEQ ID NO: 101. In some embodiments, the polynucleotide sequence of said at least one heterologous gene comprises a nucleic acid sequence having at least 55% identity to the nucleic acid sequence set forth in SEQ ID NO: 103. In some embodiments, the polynucleotide sequence of said at least one heterologous gene comprises a nucleic acid sequence having at least 55% identity to the nucleic acid sequence set forth in SEQ ID NO: 105.

In some embodiments, the polynucleotide sequence of said at least one heterologous gene comprises a nucleic acid sequence having at least 55%, 60%. 65%, 70%, 75%, 80%, 85%, 90%, 95%, 98%, or 99% identity to the nucleic acid sequence set forth in any of SEQ ID NO: 125, 127, 30, 32, 34, 36, 38, 40, 65, 80, 93, 95, 97, 99, 101, 103, or 105. In some embodiments, the polynucleotide sequence of said at least one heterologous gene comprises a nucleic acid sequence having at least 55%, 60%. 65%, 70%, 75%, 80%, 85%, 90%, 95%, 98%, 99%, or 100% coverage to the nucleic acid sequence set forth in any of SEQ ID NO: 125, 127, 30, 32, 34, 36, 38, 40, 65, 80, 93, 95, 97, 99, 101, 103, or 105.

In some embodiments, the polynucleotide sequence of said at least one heterologous gene comprises a nucleic acid sequence having at least 80% identity to the nucleic acid sequence set forth in SEQ ID NO: 125. In some embodiments, the polynucleotide sequence of said at least one heterologous gene comprises a nucleic acid sequence having at least 80% identity to the nucleic acid sequence set forth in SEQ ID NO: 127. In some embodiments, the polynucleotide sequence of said at least one heterologous gene comprises a nucleic acid sequence having at least 80% identity to the nucleic acid sequence set forth in SEQ ID NO: 30. In some embodiments, the polynucleotide sequence of said at least one heterologous gene comprises a nucleic acid sequence having at least 80% identity to the nucleic acid sequence set forth in SEQ ID NO: 32. In some embodiments, the polynucleotide sequence of said at least one heterologous gene comprises a nucleic acid sequence having at least 80% identity to the nucleic acid sequence set forth in SEQ ID NO: 34. In some embodiments, the polynucleotide sequence of said at least one heterologous gene comprises a nucleic acid sequence having at least 80% identity to the nucleic acid sequence set forth in SEQ ID NO: 36. In some embodiments, the polynucleotide sequence of said at least one heterologous gene comprises a nucleic acid sequence having at least 80% identity to the nucleic acid sequence set forth in SEQ ID NO: 38. In some embodiments, the polynucleotide sequence of said at least one heterologous gene comprises a nucleic acid sequence having at least 80% identity to the nucleic acid sequence set forth in SEQ ID NO: 40. In some embodiments, the polynucleotide sequence of said at least one heterologous gene comprises a nucleic acid sequence having at least 80% identity to the nucleic acid sequence set forth in SEQ ID NO: 45. In some embodiments, the polynucleotide sequence of said at least one heterologous gene comprises a nucleic acid sequence having at least 80% identity to the nucleic acid sequence set forth in SEQ ID NO: 46. In some embodiments, the polynucleotide sequence of said at least one heterologous gene comprises a nucleic acid sequence having at least 80% identity to the nucleic acid sequence set forth in SEQ ID NO: 47. In some embodiments, the polynucleotide sequence of said at least one heterologous gene comprises a nucleic acid sequence having at least 80% identity to the nucleic acid sequence set forth in SEQ ID NO: 51. In some embodiments, the polynucleotide sequence of said at least one heterologous gene comprises a nucleic acid sequence having at least 80% identity to the nucleic acid sequence set forth in SEQ ID NO: 53. In some embodiments, the polynucleotide sequence of said at least one heterologous gene comprises a nucleic acid sequence having at least 80% identity to the nucleic acid sequence set forth in SEQ ID NO: 55. In some embodiments, the polynucleotide sequence of said at least one heterologous gene comprises a nucleic acid sequence having at least 80% identity to the nucleic acid sequence set forth in SEQ ID NO: 57. In some embodiments, the polynucleotide sequence of said at least one heterologous gene comprises a nucleic acid sequence having at least 80% identity to the nucleic acid sequence set forth in SEQ ID NO: 59. In some embodiments, the polynucleotide sequence of said at least one heterologous gene comprises a nucleic acid sequence having at least 80% identity to the nucleic acid sequence set forth in SEQ ID NO: 61. In some embodiments, the polynucleotide sequence of said at least one heterologous gene comprises a nucleic acid sequence having at least 80% identity to the nucleic acid sequence set forth in SEQ ID NO: 63. In some embodiments, the polynucleotide sequence of said at least one heterologous gene comprises a nucleic acid sequence having at least 80% identity to the nucleic acid sequence set forth in SEQ ID NO: 65. In some embodiments, the polynucleotide sequence of said at least one heterologous gene comprises a nucleic acid sequence having at least 80% identity to the nucleic acid sequence set forth in SEQ ID NO: 80. In some embodiments, the polynucleotide sequence of said at least one heterologous gene comprises a nucleic acid sequence having at least 80% identity to the nucleic acid sequence set forth in SEQ ID NO: 93. In some embodiments, the polynucleotide sequence of said at least one heterologous gene comprises a nucleic acid sequence having at least 80% identity to the nucleic acid sequence set forth in SEQ ID NO: 95. In some embodiments, the polynucleotide sequence of said at least one heterologous gene comprises a nucleic acid sequence having at least 80% identity to the nucleic acid sequence set forth in SEQ ID NO: 97. In some embodiments, the polynucleotide sequence of said at least one heterologous gene comprises a nucleic acid sequence having at least 80% identity to the nucleic acid sequence set forth in SEQ ID NO: 99. In some embodiments, the polynucleotide sequence of said at least one heterologous gene comprises a nucleic acid sequence having at least 80% identity to the nucleic acid sequence set forth in SEQ ID NO: 101. In some embodiments, the polynucleotide sequence of said at least one heterologous gene comprises a nucleic acid sequence having at least 80% identity to the nucleic acid sequence set forth in SEQ ID NO: 103. In some embodiments, the polynucleotide sequence of said at least one heterologous gene comprises a nucleic acid sequence having at least 80% identity to the nucleic acid sequence set forth in SEQ ID NO: 105.

In some embodiments, the polynucleotide sequence of said at least one heterologous gene comprises a polynucleotide sequence encoding at least one steroidal alkaloid biosynthetic enzyme, steroidal saponin biosynthetic enzyme, or triterpenoid saponin biosynthetic enzyme. In some embodiments, the polynucleotide sequence of said at least one heterologous gene comprises a polynucleotide sequence encoding 2, 3, 4, 5, 6, 7, 8, 9, or 10, or more at least one steroidal alkaloid biosynthetic enzymes, steroidal saponin biosynthetic enzymes, or triterpenoid saponin biosynthetic enzymes, or combinations thereof.

A skilled artisan would appreciate that sequence homology encompasses similarity of sequence attributed to descent from a common ancestor. Homologous biological components (genes, proteins, structures) are called homologs. The extent to which nucleotide or protein sequences are related. The similarity between two sequences (DNA, RNA, or amino acid) can be expressed as percent sequence identity and/or percent positive substitutions.

A skilled artisan would appreciate that the term “homolog” encompasses a gene or a polypeptide (a protein) that is related to a second gene or polypeptide (protein), respectively, by descent from a common ancestral DNA or polypeptide (protein) sequence, respectively. Thus, a homolog of a gene, in some embodiments, comprises a similar nucleotide sequence to the gene. In some embodiments, a gene homolog encodes an identical polypeptide as is encoded by the gene. In some embodiments, a gene homolog encodes a polypeptide with the same functional properties as is encoded by the gene. In some embodiments, a gene homolog encodes a polypeptide that comprises a similar amino acid sequence as the polypeptide encoded by the gene. In one embodiment, the polypeptide homolog comprises a similar amino acid sequence as the polypeptide. In some embodiments, the polypeptide homolog comprises the same functional properties as the polypeptide. In some embodiments, the polypeptide homolog comprises similar functional properties as the polypeptide. In some embodiments, the polypeptide homolog comprises a same domain(s) as the polypeptide. In some embodiments, the polypeptide homolog comprises a similar domain(s) as the polypeptide.

A skilled artisan would appreciate that percent homology or percent identity may be determined, for example but no limited to, using BlastP software of the National Center of Biotechnology Information (NCBI) using default parameters. The homolog may also refer to an ortholog, a deletion, insertion, or substitution variant, including an amino acid substitution. In some embodiments, sequence identity or homology can be determined using any protein or nucleic acid sequence alignment algorithm such as Blast, ClustalW, MUSCLE, and HHpred.

A skilled artisan would appreciate that homologs, may in some embodiments, have the same activity. In some embodiments, a SOAP gene homolog has the same activity as the corresponding SOAP gene. In some embodiments, a SOAP polypeptide homolog has the same enzyme activity as the corresponding SOAP polypeptide. In some embodiments, a SOAP5 homolog has the same enzyme activities as SOAP5. In some embodiments, a GAME gene homolog has the same activity as the corresponding GAME gene. In some embodiments, a GAME polypeptide homolog has the same enzyme activity as the corresponding GAME polypeptide. In some embodiments, a GAME15 homolog has the same enzyme activities as GAME15. In certain embodiments, a homolog of a CSLG gene encodes an enzyme having cellulose synthase like G activity. In certain embodiments, a homolog of a CSLG gene encodes an enzyme having glucuronic acid transferase activity. In certain embodiments, a homolog of a CSLG gene encodes an enzyme having cellulose synthase like G activity and glucuronic acid transferase activity. FIGS. 42A and 42F provide phylogenetic trees of cellulose synthase like enzymes, wherein the cellulose synthase like G subclade includes the SOAP5 and GAME15 enzymes described herein.

In some embodiments, a genetically modified cell disclosed herein comprises a nucleic acid sequence encoding said at least one heterologous CSLG gene, wherein the nucleic acid sequence of said CSLG is set forth in any one of SEQ ID NOS: 125, 127, 30, 32, 34, 36, 38, 40, 65, 80, 93, 95, 97, 99, 101, 103, or 105; or a homolog thereof having at least 55% identity to and at least 55% coverage of, the nucleic acid sequence set forth in any one of SEQ ID NOS: 125, 127, 30, 32, 34, 36, 38, 40, 65, 80, 93, 95, 97, 99, 101, 103, or 105. In some embodiments, a genetically modified cell disclosed herein comprises a nucleic acid sequence encoding said at least one heterologous CSLG gene, wherein the nucleic acid sequence of said CSLG is set forth in any one of SEQ ID NOS: 125, 127, 30, 32, 34, 36, 38, 40, 65, 80, 93, 95, 97, 99, 101, 103, or 105; or a homolog thereof having at least 55% identity to and at least 80% coverage of, the nucleic acid sequence set forth in any one of SEQ ID NOS: 125, 127, 30, 32, 34, 36, 38, 40, 65, 80, 93, 95, 97, 99, 101, 103, or 105.

In some embodiments, a genetically modified cell disclosed herein comprises a nucleic acid sequence encoding said at least one heterologous CSLG gene, wherein the nucleic acid sequence of said CSLG is set forth in any one of SEQ ID NOS: 125, 127, 30, 32, 34, 36, 38, 40, 65, 80, 93, 95, 97, 99, 101, 103, or 105; or a homolog thereof having at least 80% identity to and at least 80% coverage of, the nucleic acid sequence set forth in any one of SEQ ID NOS: 125, 127, 30, 32, 34, 36, 38, 40, 65, 80, 93, 95, 97, 99, 101, 103, or 105.

In some embodiments, a genetically modified cell disclosed herein comprises a nucleic acid sequence encoding an at least one additional heterologous gene in addition to a heterologous CSLG gene, wherein the additional gene encodes
(a) a β-amyrin synthase, and said nucleic acid sequence set forth in SEQ ID NO: 45; or a homolog thereof having at least 80% identity to and at least 80% coverage of, the nucleic acid sequence set forth in SEQ ID NO: 45; or
(b) a cytochrome P450, said nucleic acid sequence set forth in any one of SEQ ID NO: 46, 51, or 53; or a homolog thereof having at least 80% identity to and at least 80% coverage of, the nucleic acid sequence set forth in any one of SEQ ID NO: 46, 51, or 53; or
(c) a glycosyl transferase, said nucleic acid sequence set forth in any one of SEQ ID NO: 55, 57, 59, or 61; or a homolog thereof having at least 80% identity to and at least 80% coverage of, the nucleic acid sequence set forth in any one of SEQ ID NO: 55, 57, 59, or 61; or
(d) an acetyltransferase, said nucleic acid sequence set forth in SEQ ID NO: 63; or a homolog thereof having at least 80% identity to and at least 80% coverage of, the nucleic acid sequence set forth in SEQ ID NO: 63; or
(e) a UDP-glucose 6-dehydrogenase 1, said nucleic acid sequence set forth in SEQ ID NO: 74; or a homolog thereof having at least 80% identity to and at least 80% coverage of, the nucleic acid sequence set forth in SEQ ID NO: 74; or
(f) any combination thereof of (a), (b), (c), (d), and (e).

In some embodiments, a genetically modified plant disclosed herein comprises a nucleic acid sequence encoding said at least one heterologous CSLG gene, wherein the nucleic acid sequence of said CSLG is set forth in any one of SEQ ID NOS: 125, 127, 30, 32, 34, 36, 38, 40, 65, 80, 93, 95, 97, 99, 101, 103, or 105; or a homolog thereof having at least 55% identity to and at least 55% coverage of, the nucleic acid sequence set forth in any one of SEQ ID NOS: 125, 127, 30, 32, 36, 38, 40, 65, 80, 93, 95, 97, 99, 101, 103, or 105. In some embodiments, a genetically modified plant disclosed herein comprises a nucleic acid sequence encoding said at least one heterologous CSLG gene, wherein the nucleic acid sequence of said CSLG is set forth in any one of SEQ ID NOS: 125, 127, 30, 32, 34, 36, 38, 40, 65, 80, 93, 95, 97, 99, 101, 103, or 105; or a homolog thereof having at least 55% identity to and at least 80% coverage of, the nucleic acid sequence set forth in any one of SEQ ID NOS: 125, 127, 30, 32, 36, 38, 40, 65, 80, 93, 95, 97, 99, 101, 103, or 105.

In some embodiments, a genetically modified plant disclosed herein comprises a nucleic acid sequence encoding said at least one heterologous CSLG gene, wherein the nucleic acid sequence of said CSLG is set forth in any one of SEQ ID NOS: 125, 127, 30, 32, 34, 36, 38, 40, 65, 80, 93, 95, 97, 99, 101, 103, or 105; or a homolog thereof having at least 80% identity to and at least 80% coverage of, the nucleic acid sequence set forth in any one of SEQ ID NOS: 125, 127, 30, 32, 36, 38, 40, 65, 80, 93, 95, 97, 99, 101, 103, or 105.

In some embodiments, a genetically modified plant disclosed herein comprises a nucleic acid sequence encoding an at least one additional heterologous gene in addition to a heterologous CSLG gene, wherein the additional gene encodes
(a) a β-amyrin synthase, and said nucleic acid sequence set forth in SEQ ID NO: 45; or a homolog thereof having at least 80% identity to and at least 80% coverage of, the nucleic acid sequence set forth in SEQ ID NO: 45; or
(b) a cytochrome P450, said nucleic acid sequence set forth in any one of SEQ ID NO: 46, 51, or 53; or a homolog thereof having at least 80% identity to and at least 80% coverage of, the nucleic acid sequence set forth in any one of SEQ ID NO: 46, 51, or 53; or
(c) a glycosyl transferase, said nucleic acid sequence set forth in any one of SEQ ID NO: 55, 57, 59, or 61; or a homolog thereof having at least 80% identity to and at least 80% coverage of, the nucleic acid sequence set forth in any one of SEQ ID NO: 55, 57, 59, or 61; or
(d) an acetyltransferase, said nucleic acid sequence set forth in SEQ ID NO: 63; or a homolog thereof having at least 80% identity to and at least 80% coverage of, the nucleic acid sequence set forth in SEQ ID NO: 63; or
(e) a UDP-glucose 6-dehydrogenase 1, said nucleic acid sequence set forth in SEQ ID NO: 74; or a homolog thereof having at least 80% identity to and at least 80% coverage of, the nucleic acid sequence set forth in SEQ ID NO: 74; or
(f) any combination thereof of (a), (b), (c), (d), and (e).

In some embodiments, a polynucleotide sequence comprising an at least one heterologous gene encoding an enzyme disclosed here, comprises a nucleic acid sequence having at least 55%, at least 60%, at least 65%, at least 70%, at least 75%, at least 80%, at least 85%, at least 90%, or at least 95% homology to a sequence selected from 125, 127, 30, 32, 34, 36, 38, 40, 45, 46, 47, 51, 53, 55, 57, 59, 61, 63, 65, 80, 93, 95, 97, 99, 101, 103, or 105, or a combination thereof. In some embodiments, the nucleic acid sequence of a heterologous gene encoding an enzyme disclosed herein, comprises a nucleic acid sequence having at least 55%, at least 60%, at least 65%, at least 70%, at least 75%, at least 80%, at least 85%, at least 90%, or at least 95% identity to a sequence selected from 125, 127, 30, 32, 34, 36, 38, 40, 45, 46, 47, 51, 53, 55, 57, 59, 61, 63, 65, 80, 93, 95, 97, 99, 101, 103, or 105, or a combination thereof.

In some embodiments, the nucleic acid sequence of a heterologous gene encoding an enzyme disclosed herein, comprises a nucleic acid sequence selected from selected from 125, 127, 30, 32, 34, 36, 38, 40, 45, 46, 47, 51, 53, 55, 57, 59, 61, 63, 65, 80, 93, 95, 97, 99, 101, 103, or 105, or a combination thereof.

In some embodiments, a nucleic acid sequence having at least 55%, at least 60%, at least 65%, at least 70%, at least 75%, at least 80%, at least 85%, at least 90%, or at least 95% identity to a sequence selected from 125, 127, 30, 32, 34, 36, 38, 40, 45, 46, 47, 51, 53, 55, 57, 59, 61, 63, 65, 80, 93, 95, 97, 99, 101, 103, or 105, comprises a codon optimized gene sequence.

In some embodiments, optimizing a gene entails adapting the codon usage to the codon bias of host genes, which in one embodiment comprises a plant host. In some embodiments, optimizing a gene entails adapting the codon usage to the codon bias of host genes, which in one embodiment comprises a yeast host. In some embodiments, optimizing a gene entails adapting the codon usage to the codon bias of host genes, which in one embodiment comprises a algal host. Codon optimized gene sequences may in some embodiments, improve protein expression level in living organism by increasing translational efficiency of an at least one heterologous gene as described herein. In some embodiments, codon-optimization comprising codon optimization for expression in yeast. In some embodiments, codon-optimization comprising codon optimization for expression in algae. In some embodiments, codon-optimization comprising codon optimization for expression in a plant cell.

In some embodiments, the term “optimized” encompasses a desired change, which, in one embodiment, is a change in gene expression and, in another embodiment, in protein expression. In one embodiment, optimized gene expression is optimized regulation of gene expression. In another embodiment, optimized gene expression is an increase in gene expression. According to this aspect and in one embodiment, a 2-fold through 1000-fold increase in gene expression compared to wild-type is contemplated. In another embodiment, a 2-fold to 500-fold increase in gene expression, in another embodiment, a 2-fold to 100-fold increase in gene expression, in another embodiment, a 2-fold to 50-fold increase in gene expression, in another embodiment, a 2-fold to 20-fold increase in gene expression, in another embodiment, a 2-fold to 10-fold increase in gene expression, in another embodiment, a 3-fold to 5-fold increase in gene expression is contemplated.

In another embodiment, optimized gene expression may be an increase in gene expression under particular environmental conditions. In another embodiment, optimized gene expression may comprise a decrease in gene expression, which, in one embodiment, may be only under particular environmental conditions.

In another embodiment, optimized gene expression is an increased duration of gene expression. According to this aspect and in one embodiment, a 2-fold through 1000-fold increase in the duration of gene expression compared to wild-type is contemplated. In another embodiment, a 2-fold to 500-fold increase in the duration of gene expression, in another embodiment, a 2-fold to 100-fold increase in the duration of gene expression, in another embodiment, a 2-fold to 50-fold increase in the duration of gene expression, in another embodiment, a 2-fold to 20-fold increase in the duration of gene expression, in another embodiment, a 2-fold to 10-fold increase in the duration of gene expression, in another embodiment, a 3-fold to 5-fold increase in the duration of gene expression is contemplated. In another embodiment, the increased duration of gene expression is compared to gene expression in non-vector-expressing controls, or alternatively, compared to gene expression in wild-type-vector-expressing controls.

A skilled artisan would appreciate that in some embodiments, more than one heterologous gene encoding an enzyme is expressed in a genetically modified cell. In some embodiments, the cell comprises a polynucleotide sequence encoding more than one heterologous gene. In other embodiments, when more than one heterologous gene encoding an enzyme is expressed in a genetically modified cell, multiple polynucleotide sequences may be used to encode the multiple heterologous genes, wherein a polynucleotide sequence may comprise one or more genes. In some embodiments, when more than one heterologous gene encoding an enzyme is expressed in a genetically modified cell, multiple polynucleotide sequences may be used to encode the multiple heterologous genes, wherein each polynucleotide sequence comprises one a single gene. In some embodiments, when more than one heterologous gene encoding an enzyme is expressed in a genetically modified cell, multiple polynucleotide sequences may be used to encode the multiple heterologous genes, wherein each polynucleotide sequence comprises one or more genes.

As disclosed throughout, in some embodiments a genetically modified cell comprises a plant cell. In some embodiments, a genetically modified plant cell is comprised in a plant or plant part. In other embodiments, a genetically modified cell comprises a yeast cell. In other embodiments a genetically modified cell comprises an algal cell. In other embodiments, a genetically modified cell comprises an insect cell. In other embodiments, a genetically modified cell comprises a bacterium.

Additionally, a skilled artisan would appreciate that in some embodiments, more than one heterologous gene encoding an enzyme is expressed in a genetically modified plant. In some embodiments, the plant comprises a polynucleotide sequence encoding more than one heterologous gene. In other embodiments, when more than one heterologous gene encoding an enzyme is expressed in a genetically modified plant, multiple polynucleotide sequences may be used to encode the multiple heterologous genes, wherein a polynucleotide sequence may comprise one or more genes. In some embodiments, when more than one heterologous gene encoding an enzyme is expressed in a genetically modified plant, multiple polynucleotide sequences may be used to encode the multiple heterologous genes, wherein each polynucleotide sequence comprises one a single gene. In some embodiments, when more than one heterologous gene encoding an enzyme is expressed in a genetically modified plant, multiple polynucleotide sequences may be used to encode the multiple heterologous genes, wherein each polynucleotide sequence comprises one or more genes.

In some embodiments, the amino acid sequence of an enzyme encoded by an at least one heterologous gene comprises the amino acid sequence of a steroidal alkaloid biosynthetic enzyme, a steroidal saponin biosynthetic enzyme, and/or a triterpenoid biosynthetic enzyme.

In some embodiments, the amino acid sequence of an enzyme encoded by an at least one heterologous gene is set forth in any one of SEQ ID NOS: 126, 31, 33, 35, 37, 39, 41, 48, 49, 50, 52, 54, 56, 58, 60, 62, 64, 64, 81, 94, 96, 98, 100, 102 or 104. In some embodiments, the amino acid sequence of an enzyme encoded by an at least one heterologous gene is set forth in SEQ ID NO: 126. In some embodiments, the amino acid sequence of an enzyme encoded by an at least one heterologous gene is set forth in SEQ ID NO: 31. In some embodiments, the amino acid sequence of an enzyme encoded by an at least one heterologous gene is set forth in SEQ ID NO: 33. In some embodiments, the amino acid sequence of an enzyme encoded by an at least one heterologous gene is set forth in SEQ ID NO: 35. In some embodiments, the amino acid sequence of an enzyme encoded by an at least one heterologous gene is set forth in SEQ ID NO: 37. In some embodiments, the amino acid sequence of an enzyme encoded by an at least one heterologous gene is set forth in SEQ ID NO: 39. In some embodiments, the amino acid sequence of an enzyme encoded by an at least one heterologous gene is set forth in SEQ ID NO: 41. In some embodiments, the amino acid sequence of an enzyme encoded by an at least one heterologous gene is set forth in SEQ ID NO: 48. In some embodiments, the amino acid sequence of an enzyme encoded by an at least one heterologous gene is set forth in SEQ ID NO: 49. In some embodiments, the amino acid sequence of an enzyme encoded by an at least one heterologous gene is set forth in SEQ ID NO: 50. In some embodiments, the amino acid sequence of an enzyme encoded by an at least one heterologous gene is set forth in SEQ ID NO: 52. In some embodiments, the amino acid sequence of an enzyme encoded by an at least one heterologous gene is set forth in SEQ ID NO: 54. In some embodiments, the amino acid sequence of an enzyme encoded by an at least one heterologous gene is set forth in SEQ ID NO: 56. In some embodiments, the amino acid sequence of an enzyme encoded by an at least one heterologous gene is set forth in SEQ ID NO: 58. In some embodiments, the amino acid sequence of an enzyme encoded by an at least one heterologous gene is set forth in SEQ ID NO: 60. In some embodiments, the amino acid sequence of an enzyme encoded by an at least one heterologous gene is set forth in SEQ ID NO: 62. In some embodiments, the amino acid sequence of an enzyme encoded by an at least one heterologous gene is set forth in SEQ ID NO: 64. In some embodiments, the amino acid sequence of an enzyme encoded by an at least one heterologous gene is set forth in SEQ ID NO: 66. In some embodiments, the amino acid sequence of an enzyme encoded by an at least one heterologous gene is set forth in SEQ ID NO: 69. In some embodiments, the amino acid sequence of an enzyme encoded by an at least one heterologous gene is set forth in SEQ ID NO: 81. In some embodiments, the amino acid sequence of an enzyme encoded by an at least one heterologous gene is set forth in SEQ ID NO: 94. In some embodiments, the amino acid sequence of an enzyme encoded by an at least one heterologous gene is set forth in SEQ ID NO: 96. In some embodiments, the amino acid sequence of an enzyme encoded by an at least one heterologous gene is set forth in SEQ ID NO: 98. In some embodiments, the amino acid sequence of an enzyme encoded by an at least one heterologous gene is set forth in SEQ ID NO: 100. In some embodiments, the amino acid sequence of an enzyme encoded by an at least one heterologous gene is set forth in SEQ ID NO: 102. In some embodiments, the amino acid sequence of an enzyme encoded by an at least one heterologous gene is set forth in SEQ ID NO: 104.

In some embodiments, the amino acid sequence of an enzyme encoded by an at least one heterologous gene is set forth in a sequence having at least 55% identity to the amino acid sequence set forth in any one of SEQ ID NOS:126, 31, 33, 35, 37, 39, 41, 48, 49, 50, 52, 54, 56, 58, 60, 62, 64, 64, 81, 94, 96, 98, 100, 102 or 104. In some embodiments, the amino acid sequence of an enzyme encoded by an at least one heterologous gene is set forth in a sequence having at least 55% identity to the amino acid sequence set forth in SEQ ID NO: 126. In some embodiments, the amino acid sequence of an enzyme encoded by an at least one heterologous gene is set forth in a sequence having at least 55% identity to the amino acid sequence set forth in SEQ ID NO: 31. In some embodiments, the amino acid sequence of an enzyme encoded by an at least one heterologous gene is set forth in a sequence having at least 55% identity to the amino acid sequence set forth in SEQ ID NO: 33. In some embodiments, the amino acid sequence of an enzyme encoded by an at least one heterologous gene is set forth in a sequence having at least 55% identity to the amino acid sequence set forth in SEQ ID NO: 35. In some embodiments, the amino acid sequence of an enzyme encoded by an at least one heterologous gene is set forth in a sequence having at least 55% identity to the amino acid sequence set forth in SEQ ID NO: 37. In some embodiments, the amino acid sequence of an enzyme encoded by an at least one heterologous gene is set forth in a sequence having at least 55% identity to the amino acid sequence set forth in SEQ ID NO: 39. In some embodiments, the amino acid sequence of an enzyme encoded by an at least one heterologous gene is set forth in a sequence having at least 55% identity to the amino acid sequence set forth in SEQ ID NO: 41. In some embodiments, the amino acid sequence of an enzyme encoded by an at least one heterologous gene is set forth in a sequence having at least 55% identity to the amino acid sequence set forth in SEQ ID NO: 48. In some embodiments, the amino acid sequence of an enzyme encoded by an at least one heterologous gene is set forth in a sequence having at least 55% identity to the amino acid sequence set forth in SEQ ID NO: 49. In some embodiments, the amino acid sequence of an enzyme encoded by an at least one heterologous gene is set forth in a sequence having at least 55% identity to the amino acid sequence set forth in SEQ ID NO: 50. In some embodiments, the amino acid sequence of an enzyme encoded by an at least one heterologous gene is set forth in a sequence having at least 55% identity to the amino acid sequence set forth in SEQ ID NO: 52. In some embodiments, the amino acid sequence of an enzyme encoded by an at least one heterologous gene is set forth in a sequence having at least 55% identity to the amino acid sequence set forth in SEQ ID NO: 54. In some embodiments, the amino acid sequence of an enzyme encoded by an at least one heterologous gene is set forth in a sequence having at least 55% identity to the amino acid sequence set forth in SEQ ID NO: 56. In some embodiments, the amino acid sequence of an enzyme encoded by an at least one heterologous gene is set forth in a sequence having at least 55% identity to the amino acid sequence set forth in SEQ ID NO: 58. In some embodiments, the amino acid sequence of an enzyme encoded by an at least one heterologous gene is set forth in a sequence having at least 55% identity to the amino acid sequence set forth in SEQ ID NO: 60. In some embodiments, the amino acid sequence of an enzyme encoded by an at least one heterologous gene is set forth in a sequence having at least 55% identity to the amino acid sequence set forth in SEQ ID NO: 62. In some embodiments, the amino acid sequence of an enzyme encoded by an at least one heterologous gene is set forth in a sequence having at least 55% identity to the amino acid sequence set forth in SEQ ID NO: 64. In some embodiments, the amino acid sequence of an enzyme encoded by an at least one heterologous gene is set forth in a sequence having at least 55% identity to the amino acid sequence set forth in SEQ ID NO: 66. In some embodiments, the amino acid sequence of an enzyme encoded by an at least one heterologous gene is set forth in a sequence having at least 55% identity to the amino acid sequence set forth in SEQ ID NO: 69. In some embodiments, the amino acid sequence of an enzyme encoded by an at least one heterologous gene is set forth in a sequence having at least 55% identity to the amino acid sequence set forth in SEQ ID NO: 81. In some embodiments, the amino acid sequence of an enzyme encoded by an at least one heterologous gene is set forth in a sequence having at least 55% identity to the amino acid sequence set forth in SEQ ID NO: 94. In some embodiments, the amino acid sequence of an enzyme encoded by an at least one heterologous gene is set forth in a sequence having at least 55% identity to the amino acid sequence set forth in SEQ ID NO: 96. In some embodiments, the amino acid sequence of an enzyme encoded by an at least one heterologous gene is set forth in a sequence having at least 55% identity to the amino acid sequence set forth in SEQ ID NO: 98. In some embodiments, the amino acid sequence of an enzyme encoded by an at least one heterologous gene is set forth in a sequence having at least 55% identity to the amino acid sequence set forth in SEQ ID NO: 100. In some embodiments, the amino acid sequence of an enzyme encoded by an at least one heterologous gene is set forth in a sequence having at least 55% identity to the amino acid sequence set forth in SEQ ID NO: 102. In some embodiments, the amino acid sequence of an enzyme encoded by an at least one heterologous gene is set forth in a sequence having at least 55% identity to the amino acid sequence set forth in SEQ ID NO: 104.

In some embodiments, the amino acid sequence of an enzyme encoded by an at least one heterologous gene is set forth in a sequence having at least 55%, 60%, 65%, 70%, 75%, 80%, 85%, 90%, 95%, 98%, or 99% identity to the amino acid sequence set forth in any of SEQ ID NO: 126, 31, 33, 35, 37, 39, 41, 48, 49, 50, 52, 54, 56, 58, 60, 62, 64, 64, 81, 94, 96, 98, 100, 102 or 104. In some embodiments, the amino acid sequence of an enzyme encoded by an at least one heterologous gene is set forth in a sequence having at least 55%, 60%, 65%, 70%, 75%, 80%, 85%, 90%, 95%, 98%, 99%, or 100% coverage to the amino acid sequence set forth in any of SEQ ID NO: 126, 31, 33, 35, 37, 39, 41, 48, 49, 50, 52, 54, 56, 58, 60, 62, 64, 64, 81, 94, 96, 98, 100, 102 or 104.

In some embodiments, the amino acid sequence of an enzyme encoded by an at least one heterologous gene is set forth in a sequence having at least 80% identity to the amino acid sequence set forth in any one of SEQ ID NOS:126, 31, 33, 35, 37, 39, 41, 48, 49, 50, 52, 54, 56, 58, 60, 62, 64, 64, 81, 94, 96, 98, 100, 102 or 104. In some embodiments, the amino acid sequence of an enzyme encoded by an at least one heterologous gene is set forth in a sequence having at least 80% identity to the amino acid sequence set forth in SEQ ID NO: 126. In some embodiments, the amino acid sequence of an enzyme encoded by an at least one heterologous gene is set forth in a sequence having at least 80% identity to the amino acid sequence set forth in SEQ ID NO: 31. In some embodiments, the amino acid sequence of an enzyme encoded by an at least one heterologous gene is set forth in a sequence having at least 80% identity to the amino acid sequence set forth in SEQ ID NO: 33. In some embodiments, the amino acid sequence of an enzyme encoded by an at least one heterologous gene is set forth in a sequence having at least 80% identity to the amino acid sequence set forth in SEQ ID NO: 35. In some embodiments, the amino acid sequence of an enzyme encoded by an at least one heterologous gene is set forth in a sequence having at least 80% identity to the amino acid sequence set forth in SEQ ID NO: 37. In some embodiments, the amino acid sequence of an enzyme encoded by an at least one heterologous gene is set forth in a sequence having at least 80% identity to the amino acid sequence set forth in SEQ ID NO: 39. In some embodiments, the amino acid sequence of an enzyme encoded by an at least one heterologous gene is set forth in a sequence having at least 80% identity to the amino acid sequence set forth in SEQ ID NO: 41. In some embodiments, the amino acid sequence of an enzyme encoded by an at least one heterologous gene is set forth in a sequence having at least 80% identity to the amino acid sequence set forth in SEQ ID NO: 48. In some embodiments, the amino acid sequence of an enzyme encoded by an at least one heterologous gene is set forth in a sequence having at least 80% identity to the amino acid sequence set forth in SEQ ID NO: 49. In some embodiments, the amino acid sequence of an enzyme encoded by an at least one heterologous gene is set forth in a sequence having at least 80% identity to the amino acid sequence set forth in SEQ ID NO: 50. In some embodiments, the amino acid sequence of an enzyme encoded by an at least one heterologous gene is set forth in a sequence having at least 80% identity to the amino acid sequence set forth in SEQ ID NO: 52. In some embodiments, the amino acid sequence of an enzyme encoded by an at least one heterologous gene is set forth in a sequence having at least 80% identity to the amino acid sequence set forth in SEQ ID NO: 54. In some embodiments, the amino acid sequence of an enzyme encoded by an at least one heterologous gene is set forth in a sequence having at least 80% identity to the amino acid sequence set forth in SEQ ID NO: 56. In some embodiments, the amino acid sequence of an enzyme encoded by an at least one heterologous gene is set forth in a sequence having at least 80% identity to the amino acid sequence set forth in SEQ ID NO: 58. In some embodiments, the amino acid sequence of an enzyme encoded by an at least one heterologous gene is set forth in a sequence having at least 80% identity to the amino acid sequence set forth in SEQ ID NO: 60. In some embodiments, the amino acid sequence of an enzyme encoded by an at least one heterologous gene is set forth in a sequence having at least 80% identity to the amino acid sequence set forth in SEQ ID NO: 62. In some embodiments, the amino acid sequence of an enzyme encoded by an at least one heterologous gene is set forth in a sequence having at least 80% identity to the amino acid sequence set forth in SEQ ID NO: 64. In some embodiments, the amino acid sequence of an enzyme encoded by an at least one heterologous gene is set forth in a sequence having at least 80% identity to the amino acid sequence set forth in SEQ ID NO: 66. In some embodiments, the amino acid sequence of an enzyme encoded by an at least one heterologous gene is set forth in a sequence having at least 80% identity to the amino acid sequence set forth in SEQ ID NO: 69. In some embodiments, the amino acid sequence of an enzyme encoded by an at least one heterologous gene is set forth in a sequence having at least 80% identity to the amino acid sequence set forth in SEQ ID NO: 81. In some embodiments, the amino acid sequence of an enzyme encoded by an at least one heterologous gene is set forth in a sequence having at least 80% identity to the amino acid sequence set forth in SEQ ID NO: 94. In some embodiments, the amino acid sequence of an enzyme encoded by an at least one heterologous gene is set forth in a sequence having at least 80% identity to the amino acid sequence set forth in SEQ ID NO: 96. In some embodiments, the amino acid sequence of an enzyme encoded by an at least one heterologous gene is set forth in a sequence having at least 80% identity to the amino acid sequence set forth in SEQ ID NO: 98. In some embodiments, the amino acid sequence of an enzyme encoded by an at least one heterologous gene is set forth in a sequence having at least 80% identity to the amino acid sequence set forth in SEQ ID NO: 100. In some embodiments, the amino acid sequence of an enzyme encoded by an at least one heterologous gene is set forth in a sequence having at least 80% identity to the amino acid sequence set forth in SEQ ID NO: 102. In some embodiments, the amino acid sequence of an enzyme encoded by an at least one heterologous gene is set forth in a sequence having at least 80% identity to the amino acid sequence set forth in SEQ ID NO: 104.

In some embodiments, the enzyme encoded by an at least one heterologous gene comprises an amino acid sequence having at least 55%, at least 60%, at least 65%, at least 70%, at least 75%, at least 80%, at least 85%, at least 90%, or at least 95% homology to a sequence selected from SEQ ID NOS: 126, 31, 33, 35, 37, 39, 41, 48, 49, 50, 52, 54, 56, 58, 60, 62, 64, 66, 81, 94, 96, 98, 100, 102 or 104. In some embodiments, the enzyme encoded by the at least one heterologous gene comprises the amino acid sequence having at least 55%, at least 60%, at least 65%, at least 70%, at least 75%, at least 80%, at least 85%, at least 95% identity with a sequence selected from SEQ ID NOS: 126, 31, 33, 35, 37, 39, 41, 48, 49, 50, 52, 54, 56, 58, 60, 62, 64, 66, 81, 94, 96, 98, 100, 102 or 104.

In some embodiments, the enzyme encoded by an at least one heterologous gene comprises an amino acid sequence having at least 80%, at least 85%, at least 90%, or at least 95% homology to a sequence selected from SEQ ID NOS: 126, 31, 33, 35, 37, 39, 41, 48, 49, 50, 52, 54, 56, 58, 60, 62, 64, 66, 81, 94, 96, 98, 100, 102 or 104. In some embodiments, the enzyme encoded by the at least one heterologous gene comprises the amino acid sequence having at least 80%, at least 85%, at least 95% identity with a sequence selected from SEQ ID NOS: 126, 31, 33, 35, 37, 39, 41, 48, 49, 50, 52, 54, 56, 58, 60, 62, 64, 66, 81, 94, 96, 98, 100, 102 or 104.

In some embodiments, the amino acid sequence of the encoded CSLG enzyme is set forth in any one of SEQ ID NOS: 126, 31, 33, 35, 37, 39, 41, 66, 81, 94, 96, 98, 100, 102 or 104; or a homolog thereof having at least 55% identity to and at least 55% coverage of, the amino acid sequence set forth in any one of SEQ ID NOS: 126, 31, 33, 35, 37, 39, 41, 66, 81, 94, 96, 98, 100, 102 or 104. In some embodiments, the amino acid sequence of the encoded CSLG enzyme is set forth in any one of SEQ ID NOS: 126, 31, 33, 35, 37, 39, 41, 66, 81, 94, 96, 98, 100, 102 or 104; or a homolog thereof having at least 55% identity to and at least 80% coverage of, the amino acid sequence set forth in any one of SEQ ID NOS: 126, 31, 33, 35, 37, 39, 41, 66, 81, 94, 96, 98, 100, 102 or 104.

In some embodiments, the amino acid sequence of the encoded CSLG enzyme is set forth in any one of SEQ ID NOS: 126, 31, 33, 35, 37, 39, 41, 66, 81, 94, 96, 98, 100, 102 or 104; or a homolog thereof having at least 80% identity to and at least 80% coverage of, the amino acid sequence set forth in any one of SEQ ID NOS: 126, 31, 33, 35, 37, 39, 41, 66, 81, 94, 96, 98, 100, 102 or 104.

In some embodiments, the amino acid sequence of said encoded at least one additional heterologous gene encodes a β-amyrin synthase, said amino acid sequence set forth in SEQ ID NO: 45; or a homolog thereof having at least 80% identity to and at least 80% coverage of, the amino acid sequence set forth in SEQ ID NO: 45. In some embodiments, the amino acid sequence of said encoded at least one additional heterologous gene encodes a cytochrome P450, said amino acid sequence set forth in any one of SEQ ID NO: 46, 51, or 53; or a homolog thereof having at least 80% identity to and at least 80% coverage of, the amino acid sequence set forth in any one of SEQ ID NO: 46, 51, or 53. In some embodiments, the amino acid sequence of said encoded at least one additional heterologous gene encodes a glycosyl transferase, said amino acid sequence set forth in any one of SEQ ID NO: 55, 57, 59, or 61; or a homolog thereof having at least 80% identity to and at least 80% coverage of, the amino acid sequence set forth in any one of SEQ ID NO: 55, 57, 59, or 61. In some embodiments, the amino acid sequence of said encoded at least one additional heterologous gene encodes an acetyltransferase, said amino acid sequence set forth in SEQ ID NO: 63; or a homolog thereof having at least 80% identity to and at least 80% coverage of, the amino acid sequence set forth in SEQ ID NO: 63. In some embodiments, the amino acid sequence of said encoded at least one additional heterologous gene encodes a UDP-glucose 6-dehydrogenase 1, said amino acid sequence set forth in SEQ ID NO: 74; or a homolog thereof having at least 80% identity to and at least 80% coverage of, the amino acid sequence set forth in SEQ ID NO: 74.

In some embodiments, a genetically modified plant cell has an altered expression of at least the endogenous CSLG gene compared to the expression of the endogenous CSLG in a corresponding unmodified cell. In some embodiments, a genetically modified plant cell has an altered expression of at least the endogenous CSLG gene compared to the expression of the endogenous CSLG in a corresponding unmodified cell, wherein the cell has an altered content of at least one triterpenoid saponin, derivative thereof, metabolite thereof, or biosynthetic intermediate thereof.

Altering the expression of the endogenous CSLG affects the steroidal alkaloid biosynthetic pathway, the steroidal saponin biosynthetic pathway, and/or the triterpenoid saponin biosynthetic pathway, and in some embodiments, results in concomitant alteration, respectively, in the steroidal alkaloid profile or intermediates of that pathway, in the steroidal saponin profile or intermediates of that pathway, or in the triterpenoid saponin profile or intermediates of that pathway.

In some embodiments, the altered expression in a genetically modified cells comprises increased CSLG expression compared with a corresponding unmodified cell. In some embodiments, increased expression results in an increased amount of the encoded enzyme. In some embodiments, increased expression of CSLG results in the alteration in the steroidal alkaloid profile comprising an increase in at least one steroidal alkaloid or an intermediate thereof. In some embodiments, increased expression of CSLG results in the alteration in the steroidal saponin profile comprising an increase in at least one steroidal saponin or an intermediate thereof. In some embodiments, increased expression of CSLG results in the alteration in the triterpenoid saponin profile comprising an increase in at least one triterpenoid saponin or an intermediate thereof.

In some embodiments, the altered expression in a genetically modified cells comprises decreased CSLG expression compared with a corresponding unmodified cell. In some embodiments, decreased expression results in a decreased amount of the encoded enzyme. In some embodiments, decreased expression of CSLG results in the alteration in the steroidal alkaloid profile comprising a decrease in at least one steroidal alkaloid or an intermediate thereof. In some embodiments, decreased expression of CSLG results in the alteration in the steroidal saponin profile comprising a decrease in at least one steroidal saponin or an intermediate thereof. In some embodiments, decreased expression of CSLG results in the alteration in the triterpenoid saponin profile comprising a decrease in at least one triterpenoid saponin or an intermediate thereof.

In some embodiments, the at least one cell having an altered endogenous CSLG gene expression comprises a plant cell. In some embodiments, the at least one cell having an altered endogenous CSLG gene expression comprises a plant cell comprised within a plant or plant part. In some embodiments, the at least one cell having an altered endogenous CSLG gene expression is a result of a mutation in said CSLG gene. In some embodiments, said mutation comprises at least one or more point mutations, or an insertion, or a deletion, or any combination thereof, wherein said expressed CSLG enzyme has increased stability or increased activity or both and the altered content comprises an increased amount of the at least one triterpenoid saponin, a derivative thereof, a metabolite thereof, or a biosynthetic intermediate thereof compared to the corresponding unmodified plant. In some embodiments, said mutation comprises at least one or more point mutations, or an insertion, or a deletion, or any combination thereof, wherein said expressed CSLG enzyme has decreased stability or decreased activity or both and the altered content comprises a decreased amount of the at least one triterpenoid saponin, a derivative thereof, a metabolite thereof, or a biosynthetic intermediate thereof compared to the corresponding unmodified plant.

According to certain embodiments, expression of the endogenous CSLG gene is altered, the altering comprising mutagenizing the CSLG gene, wherein the mutagenesis comprises introduction of one or more point mutations, insertions, deletions, or genome editing, or use of a bacterial CRISPR/CAS system, or a combination thereof. According to certain embodiments, expression of the gene encoding the CLSG enzyme is increased compared to its expression in the corresponding unmodified plant, thereby the genetically modified plant comprises increased content of at least one steroidal alkaloid, derivative thereof, metabolite thereof, or biosynthetic intermediate thereof, increased content of at least one steroidal saponin, derivative thereof, metabolite thereof, or biosynthetic intermediate thereof, or increased content of at least one triterpenoid saponin, derivative thereof, metabolite thereof, or biosynthetic intermediate thereof, compared to the corresponding unmodified plant. According to certain embodiments, expression of the gene encoding the CLSG enzyme is decreased compared to its expression in the corresponding unmodified plant, thereby the genetically modified plant comprises decreased content at least one steroidal alkaloid, derivative thereof, metabolite thereof, or biosynthetic intermediate thereof; decreased content at least one steroidal saponin, derivative thereof, metabolite thereof, or biosynthetic intermediate thereof, or decreased content at least one triterpenoid saponin, derivative thereof, metabolite thereof, or biosynthetic intermediate thereof, compared to the corresponding unmodified plant.

In some embodiments, derivatives of steroidal alkaloids, steroidal saponins, or triterpenoid saponins comprise glycosylated derivatives of, respectively, steroidal alkaloids, steroidal saponins, or triterpenoid saponins.

Inserting a mutation to the CSLG gene, including deletions, insertions, site specific mutations, zinc-finger nucleases and the like can be used for example for upregulation of gene expression, increased stability of the expressed CSLG enzyme, increased activity of the CSLG enzyme, down-regulation of the gene expression, decreased stability of the expressed CSLG enzyme, decreased activity of the CSLG enzyme, or elimination of activity of the CSLG enzyme.

According to certain embodiments, a mutated CSLG gene comprises a polynucleotide having at least one mutation in the nucleic acid sequence of a CSLG gene, wherein the nucleic acid sequence of said CSLG is set forth in any one SEQ ID NOS: 126, 31, 33, 35, 37, 39, 41, 65, 80, 93, 95, 97, 99, 101, 103, or 105, or a complementary sequence thereof.

One of ordinary skill in the art would appreciate that a genetically modified plant may encompass a plant comprising at least one cell genetically modified by man. In some embodiments, the genetic modification includes modification of an endogenous gene(s), for example by introducing mutation(s) deletions, insertions, transposable element(s) and the like into an endogenous polynucleotide or gene of interest. Additionally, or alternatively, in some embodiments, the genetic or, modification includes transforming at least one plant cell with a heterologous polynucleotide or multiple heterologous polynucleotides. The skilled artisan would appreciate that a genetically modified plant comprising transforming at least one plant cell with a heterologous polynucleotide or multiple heterologous polynucleotides may in certain embodiments be termed a “transgenic plant” or a “genetically modified plant” having the same meanings and qualities.

Altering the expression of an endogenous CSLG gene may in certain embodiments be achieved by the introduction of one or more point mutations into a nucleic acid molecule encoding the corresponding protein. Mutations can be introduced using, for example, site-directed mutagenesis (see, e.g., Wu Ed., 1993 Meth. In Enzymol. Vol. 217, San Diego: Academic Press; Higuchi, “Recombinant PCR” in Innis et al. Eds., 1990 PCR Protocols, San Diego: Academic Press, Inc). Such mutagenesis can be used to introduce a specific, desired amino acid insertion, deletion or substitution. Several technologies for targeted mutagenesis are based on the targeted induction of double-strand breaks (DSBs) in the genome followed by error-prone DNA repair. Mostly commonly used for genome editing by these methods are custom designed nucleases, including zinc finger nucleases, endonucleases including meganucleases, and Xanthomonas-derived transcription activator-like effector nuclease (TALEN) enzymes.

In some embodiments, when the expression of the CSLG gene is altered, said altering comprises mutagenizing CSLG gene, said mutation present within a coding region of said CSLG gene, or a regulatory sequence of said CSLG gene, or a combination thereof.

Various types of mutagenesis can be used to modify CSLG and the encoded enzyme thereof in order to produce conservative or non-conservative variants. Any available mutagenesis procedure can be used. In some embodiments, the mutagenesis procedure comprises site-directed point mutagenesis. In some embodiments, the mutagenesis procedure comprises random point mutagenesis. In some embodiments, the mutagenesis procedure comprises in vitro or in vivo homologous recombination (DNA shuffling). In some embodiments, the mutagenesis procedure comprises mutagenesis using uracil-containing templates. In some embodiments, the mutagenesis procedure comprises oligonucleotide-directed mutagenesis. In some embodiments, the mutagenesis procedure comprises phosphorothioate-modified DNA mutagenesis. In some embodiments, the mutagenesis procedure comprises mutagenesis using gapped duplex DNA. In some embodiments, the mutagenesis procedure comprises point mismatch repair. In some embodiments, the mutagenesis procedure comprises mutagenesis using repair-deficient host strains. In some embodiments, the mutagenesis procedure comprises restriction-selection and restriction-purification. In some embodiments, the mutagenesis procedure comprises deletion mutagenesis. In some embodiments, the mutagenesis procedure comprises mutagenesis by total gene synthesis. In some embodiments, the mutagenesis procedure comprises double-strand break repair. In some embodiments, the mutagenesis procedure comprises mutagenesis by chimeric constructs. In some embodiments, the mutagenesis procedure comprises mutagenesis by CRISPR/Cas. In some embodiments, the mutagenesis procedure comprises mutagenesis by a meganuclease. In some embodiments, the mutagenesis procedure comprises mutagenesis by zinc-finger nucleases (ZFN). In some embodiments, the mutagenesis procedure comprises mutagenesis by transcription activator-like effector nucleases (TALEN). In some embodiments, the mutagenesis procedure comprises any other mutagenesis procedure known to a person skilled in the art.

In some embodiments, mutagenesis can be guided by known information about the naturally occurring molecule and/or the mutated molecule. By way of example, this known information may include sequence, sequence comparisons, physical properties, crystal structure and the like. In some embodiments, the mutagenesis is essentially random. In some embodiments the mutagenesis procedure is DNA shuffling.

A skilled artisan would appreciate that clustered regularly interspaced short palindromic repeats (CRISPR)/CRISPR associated protein (Cas) system comprises genome engineering tools based on the bacterial CRISPR/Cas prokaryotic adaptive immune system. This RNA-based technology is very specific and allows targeted cleavage of genomic DNA guided by a customizable small noncoding RNA, resulting in gene modifications by both non-homologous end joining (NHEJ) and homology-directed repair (HDR) mechanisms (Belhaj K. et al., 2013. Plant Methods 2013, 9:39). In some embodiments, a CRISPR/Cas system comprises a CRISPR/Cas9 system.

In some embodiments, a CRISPR/Cas system comprises a single-guide RNA (sgRNA) and/or a Cas protein known in the art. In some embodiments, a CRISPR/Cas system comprises a single-guide RNA (sgRNA) and/or a Cas protein newly created to cleave at a preselected site. The skilled artisan would appreciate that the terms “single-guide RNA”, “sgRNA”, and “gRNA” are interchangeable having all the same qualities and meanings, wherein an sgRNA may encompass a chimeric RNA molecule which is composed of a CRISPR RNA (crRNA) and trans-encoded CRISPR RNA (tracrRNA). In some embodiments, a crRNA is complementary to a preselected region of GAME15 DNA, wherein the crRNA “targets” the CRISPR associated polypeptide (Cas) nuclease protein to the preselected target site.

In some embodiments, the length of crRNA sequence complementary is 19-22 nucleotides long e.g., 19-22 consecutive nucleotides complementary to the target site. In another embodiment, the length of crRNA sequence complementary to the region of DNA is about 15-30 nucleotides long. In another embodiment, the length of crRNA sequence complementary to the region of DNA is about 15, 16, 17, 18, 19, 20, 21, 22, 23, 24, 25, 26, 27, 28, 29, or 30 nucleotides long. In another embodiment, the length of crRNA sequence complementary to the region of DNA is 20 nucleotides long. In some embodiments, the crRNA is located at the 5′ end of the sgRNA molecule. In another embodiment, the crRNA comprises 100% complementation within the preselected target sequence. In another embodiment, the crRNA comprises at least 80% complementation within the preselected target sequence. In another embodiment, the crRNA comprises at least 85% complementation within the preselected target sequence. In another embodiment, the crRNA comprises at least 90% complementation within the preselected target sequence. In another embodiment, the crRNA comprises at least 95% complementation within the preselected target sequence. In another embodiment, the crRNA comprises at least 97% complementation within the preselected target sequence. In another embodiment, the crRNA comprises at least 99% complementation within the preselected target sequence. In another embodiment, a tracrRNA is 100-300 nucleotides long and provides a binding site for the Cas nuclease, e.g., a Cas9 protein forming the CRISPR/Cas9 complex.

In one embodiment, a mutagenesis system comprises a CRISPR/Cas system. In another embodiment, a CRISPR/Cas system comprises a Cas nuclease and a gRNA molecule, wherein said gRNA molecule binds within said preselected endogenous target site thereby guiding said Cas nuclease to cleave the DNA within said preselected endogenous target site.

In some embodiments, a CRISPR/Cas system comprise an enzyme system including a guide RNA sequence (“gRNA” or “sgRNA”) that contains a nucleotide sequence complementary or substantially complementary to a region of a target polynucleotide, for example a preselected endogenous target site, and a protein with nuclease activity.

In another embodiment, a CRISPR/Cas system comprises a Type I CRISPR-Cas system, or a Type II CRISPR-Cas system, or a Type III CRISPR-Cas system, or derivatives thereof. In another embodiment, a CRISPR-Cas system comprises an engineered and/or programmed nuclease system derived from naturally accruing CRISPR-Cas systems. In another embodiment, a CRISPR-Cas system comprises engineered and/or mutated Cas proteins. In another embodiment, a CRISPR-Cas system comprises engineered and/or programmed guide RNA.

A skilled artisan would appreciate that a guide RNA may contain nucleotide sequences other than the region complementary or substantially complementary to a region of a targetDNA sequence, for example a preselected endogenous target site. In another embodiment, a guide RNA comprises a crRNA or a derivative thereof. In another embodiment, a guide RNA comprises a crRNA: tracrRNA chimera.

In another embodiment, a gRNA molecule comprises a domain that is complementary to and binds to a preselected endogenous target site on at least one homologous chromosome. In another embodiment, a gRNA molecule comprises a domain that is complementary to and binds to a polymorphic allele on at least one homologous chromosome. In another embodiment, a gRNA molecule comprises a domain that is complementary to and binds to a preselected endogenous target site on both homologous chromosomes. In another embodiment, a gRNA molecule comprises a domain that is complementary to and binds to polymorphic alleles on both homologous chromosomes.

Cas enzymes comprise RNA-guided DNA endonuclease able to make double-stranded breaks (DSB) in DNA. The term “Cas enzyme” may be used interchangeably with the terms “CRISPR-associated endonucleases” or “CRISPR-associated polypeptides” having all the same qualities and meanings. In one embodiment, a Cas enzyme is selected from the group comprising Cas1, Cas1B, Cas2, Cas3, Cas4, Cas5, Cas6, Cas7, Cas8, Cas9, Cas10, C2cl, CasX, NgAgo, Cpf1, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx4, Csx10, Csx16, CsaX, Csx3, Csx1, Csx15, Csf1, Csf2, Csf3, and Csf4, or homologs thereof, or modified versions thereof. In another embodiment, a Cas enzyme comprises Cas9. In another embodiment, a Cas enzyme comprises Cas1. In another embodiment, a Cas enzyme comprises Cas1B. In another embodiment, a Cas enzyme comprises Cas2. In another embodiment, a Cas enzyme comprises Cas3. In another embodiment, a Cas enzyme comprises Cas4. In another embodiment, a Cas enzyme comprises Cas5. In another embodiment, a Cas enzyme comprises Cas6. In another embodiment, a Cas enzyme comprises Cas7. In another embodiment, a Cas enzyme comprises Cas8. In another embodiment, a Cas enzyme comprises Cas10. In another embodiment, a Cas enzyme comprises Cpf1. In another embodiment, a Cas enzyme comprises Csy1. In another embodiment, a Cas enzyme comprises Csy2. In another embodiment, a Cas enzyme comprises Csy3. In another embodiment, a Cas enzyme comprises Cse1. In another embodiment, a Cas enzyme comprises Cse2. In another embodiment, a Cas enzyme comprises Csc1. In another embodiment, a Cas enzyme comprises Csc2. In another embodiment, a Cas enzyme comprises Csa5. In another embodiment, a Cas enzyme comprises Csn2. In another embodiment, a Cas enzyme comprises Csm2. In another embodiment, a Cas enzyme comprises Csm3. In another embodiment, a Cas enzyme comprises Csm4. In another embodiment, a Cas enzyme comprises Csm5. In another embodiment, a Cas enzyme comprises Csm6. In another embodiment, a Cas enzyme comprises Cmr1. In another embodiment, a Cas enzyme comprises Cmr3. In another embodiment, a Cas enzyme comprises Cmr4. In another embodiment, a Cas enzyme comprises Cmr5. In another embodiment, a Cas enzyme comprises Cmr6. In another embodiment, a Cas enzyme comprises Csb1. In another embodiment, a Cas enzyme comprises Csb2. In another embodiment, a Cas enzyme comprises Csb3. In another embodiment, a Cas enzyme comprises Csx17. In another embodiment, a Cas enzyme comprises Csx14. In another embodiment, a Cas enzyme comprises Csx10. In another embodiment, a Cas enzyme comprises Csx16, CsaX. In another embodiment, a Cas enzyme comprises Csx3. In another embodiment, a Cas enzyme comprises Csx1, Csx15, Csf1. In another embodiment, a Cas enzyme comprises Csf2. In another embodiment, a Cas enzyme comprises Csf3. In another embodiment, a Cas enzyme comprises Csf4. In another embodiment, a Cas enzyme comprises Cpf1. In another embodiment, a Cas enzyme comprises C2cl. In another embodiment, a Cas enzyme comprises CasX. In another embodiment, a Cas enzyme comprises NgAgo. In another embodiment, a Cas enzyme is Cas homologue. In another embodiment, a Cas enzyme is a Cas orthologue. In another embodiment, a Cas enzyme is a modified Cas enzyme. In another embodiment, a Cas enzyme is any CRISPR-associated endonucleases known in the art.

A skilled artisan would appreciate that the terms “zinc finger nuclease” or “ZFN” are interchangeable having all the same meanings and qualities, wherein a ZFN encompasses a chimeric protein molecule comprising at least one zinc finger DNA binding domain operatively linked to at least one nuclease capable of double-strand cleaving of DNA. In some embodiments, a ZFN system comprises a ZFN known in the art. In some embodiments, a ZFN system comprises a ZFN newly created to cleave a preselected site.

In some embodiments, a ZFN creates a double-stranded break at a preselected endogenous target site. In some embodiments, a ZFN comprises a DNA-binding domain and a DNA-cleavage domain, wherein the DNA binding domain is comprised of at least one zinc finger and is operatively linked to a DNA-cleavage domain. In another embodiment, a zinc finger DNA-binding domain is at the N-terminus of the chimeric protein molecule and the DNA-cleavage domain is located at the C-terminus of the molecule. In another embodiment, a zinc finger DNA-binding domain is at the C-terminus of the chimeric protein molecule and the DNA-cleavage domain is located at the N-terminus of the molecule. In another embodiment, a zinc finger binding domain encompasses the region in a zinc finger nuclease that is capable of binding to a target locus, for example a preselected endogenous target site as disclosed herein. In another embodiment, a zinc finger DNA-binding domain comprises a protein domain that binds to a preselected endogenous target site on at least one homologous chromosome. In another embodiment, a zinc finger DNA-binding domain comprises a protein domain that binds to a polymorphic allele on at least one homologous chromosome. In another embodiment, a zinc finger DNA-binding domain comprises a protein domain that binds to a preselected endogenous target site on both homologous chromosomes. In another embodiment, a zinc finger DNA-binding domain comprises a protein domain that binds to polymorphic alleles on both homologous chromosomes.

The skilled artisan would appreciate that the term “chimeric protein” is used to describe a protein that has been expressed from a DNA molecule that has been created by operatively joining two or more DNA fragments. The DNA fragments may be from the same species, or they may be from a different species. The DNA fragments may be from the same or a different gene. The skilled artisan would appreciate that the term “DNA cleavage domain” of a ZFN encompasses the region in the zinc finger nuclease that is capable of breaking down the chemical bonds between nucleic acids in a nucleotide chain. Examples of proteins containing cleavage domains include restriction enzymes, topoisomerases, recombinases, integrases and DNAses.

A skilled artisan would appreciate that endonucleases include meganucleases, also known as homing endonucleases (HEases), which like restriction endonucleases, bind and cut at a specific DSB target site, however the DSB target sites for meganucleases are typically longer, about 18 bp or more. Meganuclease domains, structure and function are known, see for example, Guhan and Muniyappa (2003) Crit Rev Biochem Mol Biol 38:199-248; Lucas et al., (2001) Nucleic Acids Res 29:960-9; Jurica and Stoddard, (1999) Cell Mol Life Sci 55:1304-26; Stoddard, (2006) Q Rev Biophys 38:49-95; and Moure et al., (2002) Nat Struct Biol 9:764. In some examples a naturally occurring variant, and/or engineered derivative meganudease is used. Any meganudease can be used herein, including, but not limited to, l-Scel, l-Scell, l-Scelll, l-ScelV, l-SceV, l-SceVI, l-SceVII, l-Ceul, l-CeuAIIP, l-Crel, I-CrepsblP, l-CrepsbllP, l-CrepsblllP, l-CrepsblVP, l-Tlil, l-Ppol, Pl-Pspl, F-Scel, F-Scell, F-Suvl, F-Tevl, F-Tevll, l-Amal, l-Anil, l-Chul, l-Cmoel, l-Cpal, l-Cpall, I-Csml, l-Cvul, l-CvuAIP, l-Ddil, l-Ddill, l-Dirl, l-Dmol, l-Hmul, l-Hmull, l-HsNIP, l-Llal, l-Msol, l-Naal, l-Nanl, l-NcllP, l-NgrIP, l-Nitl, l-Njal, l-Nsp236IP, l-Pakl, l-PbolP, I-PculP, l-PcuAI, l-PcuVI, l-PgrIP, l-PoblP, l-Porl, l-PorllP, l-PbpIP, l-SpBetalP, I-Scal, l-SexlP, l-SnelP, l-Spoml, l-SpomCP, l-SpomlP, l-SpomllP, l-SqulP, I-Ssp6803l, l-SthPhiJP, l-SthPhiST3P, l-SthPhiSTe3bP, l-TdelP, l-Tevl, l-Tevll, I-Tevlll, l-UarAP, l-UarHGPAIP, l-UarHGPA13P, l-VinIP, l-ZbilP, Pl-Mtul, PI-MtuHIP PI-MtuHIIP, Pl-Pful, Pl-Pfull, Pl-Pkol, Pl-Pkoll, PI-Rma43812IP, PI-SpBetalP, Pl-Scel, Pl-Tful, Pl-Tfull, Pl-Thyl, Pl-Tlil, Pl-Tlill, or any active variants or fragments thereof. TAL effector nucleases can be used to make double-strand breaks at specific target sequences in the genome of a plant or other organism. TAL effector nucleases can be created by fusing a native or engineered transcription activatorlike (TAL) effector, or functional part thereof, to the catalytic domain of an endonuclease, such as, for example, Fokl. The unique, modular TAL effector DNA binding domain allows for the design of proteins with potentially any given DNA recognition specificity. Thus, the DNA binding domains of the TAL effector nucleases can be engineered to recognize specific DNA target sites and thus, used to make double-strand breaks at desired target sequences. See, WO 2010/079430; Morbitzer et al. (2010) PNAS 10.1073/pnas.1013133107; Scholze & Boch (2010) Virulence 1:428-432; Christian et al. Genetics (2010) 186:757-761; Li et al. (2010) Nuc. Acids Res. (2010) doi:10.1093/nar/gkg704; and Miller et al. (2011) Nature Biotechnology 29:143-148; all of which are herein incorporated by reference.

In some embodiments, a TALEN system comprises a TAL effector DNA binding domain and a DNA cleavage domain, wherein said TAL effector DNA binding domain binds within said preselected endogenous target site, thereby targeting the DNA cleavage domain to cleave the DNA within said preselected endogenous target site.

A skilled artisan would appreciate that the terms “transcription activator-like effector nuclease”, “TALEN”, and “TAL effector nuclease” may be used interchangeably having all the same meanings and qualities, wherein a TALEN encompasses a nuclease capable of recognizing and cleaving its target site, for example a preselected endogenous target site as disclosed herein. In another embodiment, a TALEN comprises a fusion protein comprising a TALE domain and a nucleotide cleavage domain. In another embodiment, a TALE domain comprises a protein domain that binds to a nucleotide in a sequence-specific manner through one or more TALE-repeat modules. A skilled artisan would recognize that TALE-repeat modules comprise a variable number of about 34 amino acid repeats that recognize plant DNA sequences. Further, repeat modules can be rearranged according to a simple cipher to target new DNA sequences. In another embodiment, a TALE domain comprises a protein domain that binds to a preselected endogenous target site on at least one homologous chromosome. In another embodiment, a TALE domain comprises a protein domain that binds to a polymorphic allele on at least one homologous chromosome. In another embodiment, a TALE domain comprises a protein domain that binds to a preselected endogenous target site on both homologous chromosomes. In another embodiment, a TALE domain comprises a protein domain that binds to polymorphic alleles on both homologous chromosomes.

In one embodiment, a TALE domain comprises at least one of the TALE-repeat modules. In another embodiment, a TALE domain comprises from one to thirty TALE-repeat modules. In another embodiment, a TALE domain comprises more than thirty repeat modules. In another embodiment, a TALEN fusion protein comprises an N-terminal domain, one or more of TALE-repeat modules followed by a half-repeat module, a linker, and a nucleotide cleavage domain.

Chemical mutagenesis using an agent such as Ethyl Methyl Sulfonate (EMS) can be employed to obtain a population of point mutations and screen for mutants of the CSGL gene that may become silent or down-regulated. In plants, methods relaying on introgression of genes from natural populations can be used. Cultured and wild types species are crossed repetitively such that a plant comprising a given segment of the wild genome is isolated. Certain plant species, for example, maize (corn) and snapdragon, have natural transposons. These transposons are either autonomous, i.e. the transposase is located within the transposon sequence or non-autonomous, without a transposase. A skilled person can cause transposons to “jump” and create mutations. Alternatively, a nucleic acid sequence can be synthesized having random nucleotides at one or more predetermined positions to generate random amino acid substituting.

In some embodiments, the expression of an endogenous CSLG gene can be altered by the introduction of one or more point mutations into their regulatory sequences. In some embodiments, the expression of a heterologous CSLG gene can be altered by the introduction of one or more point mutations into their regulatory sequences.

A skilled artisan would appreciate that “regulatory sequences” refers to nucleotide sequences located upstream (5′ non-coding sequences), within, or downstream (3′ non-coding sequences) of a coding sequence, and which influence the transcription, RNA processing or stability, or translation of the associated coding sequence. In some embodiments, regulatory sequences comprise promoters. In some embodiments, regulatory sequences comprise translation leader sequences. In some embodiments, regulatory sequences comprise introns. In some embodiments, regulatory sequences comprise polyadenylation recognition sequences. In some embodiments, regulatory sequences comprise RNA processing sites. In some embodiments, regulatory sequences comprise effector binding sites. In some embodiments, regulatory sequences comprise stem-loop structures.

A skilled artisan would appreciate that “promoter” refers to a DNA sequence capable of controlling the expression of a coding sequence or functional RNA. In some embodiments, a coding sequence is located 3′ to a promoter sequence. It is understood by those skilled in the art that different promoters may direct the expression of a gene in different tissues or cell types, or at different stages of development, or in response to different environmental or physiological conditions. In some embodiments, the promoter comprises a constitutive promoter, i.e., a promoter that causes a gene to be expressed in most cell types at most times. In some embodiments, the promoter comprises a regulated promoter, i.e., a promoter that causes a gene to be expressed in response to sporadic specific stimuli. It is further recognized that in many cases the exact boundaries of regulatory sequences have not been completely defined yet.

A skilled artisan would appreciate that the term “3′ non-coding sequences” or “transcription terminator” refers to DNA sequences located downstream of a coding sequence. In some embodiments, 3′ non-coding sequences comprise polyadenylation recognition sequences. In some embodiments, 3′ non-coding sequences comprise sequences encoding regulatory signals capable of affecting mRNA processing. In some embodiments, 3′ non-coding sequences comprise sequences encoding regulatory signals capable of affecting gene expression. The polyadenylation signal is usually characterized by affecting the addition of polyadenylic acid tracts to the 3′ end of the mRNA precursor. In some embodiments, mutations in the 3′ non-coding sequences affect gene transcription. In some embodiments, mutations in the 3′ non-coding sequences affect RNA processing. In some embodiments, mutations in the 3′ non-coding sequences affect gene stability. In some embodiments, mutations in the 3′ non-coding sequences affect translation of the associated coding sequence.

According to certain embodiments, expression of the gene encoding the CSLG enzyme is reduced compared to its expression in the corresponding unmodified plant, thereby the genetically modified plant comprises reduced content of at least one steroidal alkaloid, derivative thereof, metabolite thereof, or biosynthetic intermediate; reduced content of at least one steroidal saponin, derivative thereof, metabolite thereof, or biosynthetic intermediate; or reduced content of at least one triterpenoid saponin, derivative thereof, metabolite thereof, or biosynthetic intermediate, compared to the corresponding unmodified plant.

In some embodiments, derivatives of steroidal alkaloids, steroidal saponins, or triterpenoid saponins comprise glycosylated derivatives of, respectively, steroidal alkaloids, steroidal saponins, or triterpenoid saponins.

According to certain embodiments, the genetically modified plant is a transgenic plant comprising at least one cell comprising at least one silencing molecule targeted to a polynucleotide encoding a CLSG enzyme. According to certain embodiments, the transgenic plant comprises a polynucleotide encoding a CLSG enzyme, wherein expression of the polynucleotide expression is selectively silenced, repressed, or reduced. According to certain embodiments, the transgenic plant comprises a polynucleotide encoding a CSLG enzyme, wherein the polynucleotide has been selectively edited by deletion, insertion, or modification to silence, repress, or reduce expression thereof, or wherein the genetically modified plant is a progeny of the gene edited plant.

According to certain embodiments, the transgenic plant comprises at least one cell comprising at least one silencing molecule targeted to a CSLG gene.

In some embodiments, said endogenous CSLG gene is selectively silenced, repressed, or has reduced expression and said altered content comprises a reduced amount of the at least one steroidal alkaloid, a derivative thereof, a metabolite thereof, or a biosynthetic intermediate thereof, a reduced amount of the at least one steroidal saponin, a derivative thereof, a metabolite thereof, or a biosynthetic intermediate thereof, or a reduced amount of the at least one triterpenoid saponin, a derivative thereof, a metabolite thereof, or a biosynthetic intermediate thereof, compared to the corresponding unmodified plant. wherein when said endogenous CSLG gene is selectively silenced, repressed, or has reduced expression, said cell further comprises at least one silencing molecule targeted to the polynucleotide encoding said CSLG gene, wherein the silencing molecule is selected from an RNA interference molecule or an antisense molecule, or wherein the silencing molecule is a component of a viral induced gene silencing system.

According to certain embodiments, the transgenic plant comprises at least one cell comprising at least one silencing molecule targeted to the nucleic acid sequence set forth in any one of SEQ ID NOS: 125, 127, 30, 32, 34, 36, 38, 40, 65, 80, 93, 95, 97, 99, 101, 103, or 105.

According to certain embodiments, the transgenic plant comprises at least one cell comprising at least one silencing molecule targeted to the nucleic acid sequence set forth in SEQ ID NO: 125. According to certain embodiments, the transgenic plant comprises at least one cell comprising at least one silencing molecule targeted to the nucleic acid sequence set forth in SEQ ID NO: 127. According to certain embodiments, the transgenic plant comprises at least one cell comprising at least one silencing molecule targeted to the nucleic acid sequence set forth in SEQ ID NO: 30. According to certain embodiments, the transgenic plant comprises at least one cell comprising at least one silencing molecule targeted to the nucleic acid sequence set forth in SEQ ID NO: 32. According to certain embodiments, the transgenic plant comprises at least one cell comprising at least one silencing molecule targeted to the nucleic acid sequence set forth in SEQ ID NO: 34. According to certain embodiments, the transgenic plant comprises at least one cell comprising at least one silencing molecule targeted to the nucleic acid sequence set forth in SEQ ID NO: 36. According to certain embodiments, the transgenic plant comprises at least one cell comprising at least one silencing molecule targeted to the nucleic acid sequence set forth in SEQ ID NO: 38. According to certain embodiments, the transgenic plant comprises at least one cell comprising at least one silencing molecule targeted to the nucleic acid sequence set forth in SEQ ID NO: 40. According to certain embodiments, the transgenic plant comprises at least one cell comprising at least one silencing molecule targeted to the nucleic acid sequence set forth in SEQ ID NO: 65. According to certain embodiments, the transgenic plant comprises at least one cell comprising at least one silencing molecule targeted to the nucleic acid sequence set forth in SEQ ID NO: 80. According to certain embodiments, the transgenic plant comprises at least one cell comprising at least one silencing molecule targeted to the nucleic acid sequence set forth in SEQ ID NO: 93. According to certain embodiments, the transgenic plant comprises at least one cell comprising at least one silencing molecule targeted to the nucleic acid sequence set forth in SEQ ID NO: 95. According to certain embodiments, the transgenic plant comprises at least one cell comprising at least one silencing molecule targeted to the nucleic acid sequence set forth in SEQ ID NO: 97. According to certain embodiments, the transgenic plant comprises at least one cell comprising at least one silencing molecule targeted to the nucleic acid sequence set forth in SEQ ID NO: 99. According to certain embodiments, the transgenic plant comprises at least one cell comprising at least one silencing molecule targeted to the nucleic acid sequence set forth in SEQ ID NO: 101. According to certain embodiments, the transgenic plant comprises at least one cell comprising at least one silencing molecule targeted to the nucleic acid sequence set forth in SEQ ID NO: 103. According to certain embodiments, the transgenic plant comprises at least one cell comprising at least one silencing molecule targeted to the nucleic acid sequence set forth in SEQ ID NO: 105.

According to certain embodiments, the silencing molecule is selected from the group consisting of an RNA interference molecule and an antisense molecule, or wherein the silencing molecule is a component of a viral induced gene silencing system. According to certain embodiments, the silencing molecule comprises a polynucleotide having a nucleic acid sequence substantially complementary to a region of a CSLG gene having the nucleic acid sequence set forth in any one SEQ ID NOS: 125, 127, 30, 32, 34, 36, 38, 40, 65, 80, 93, 95, 97, 99, 101, 103, or 105, or a complementary sequence thereof. In some embodiments, an antisense molecule silencing, repressing, or reducing the expression of CSLG in a plant or plant cell comprises a polynucleotide having a nucleic acid sequence substantially complementary to a region of a CSLG gene having the nucleic acid sequence set forth in any one SEQ ID NOS: 125, 127, 30, 32, 34, 36, 38, 40, 65, 80, 93, 95, 97, 99, 101, 103, or 105, or a complementary sequence thereof. In some embodiments, an antisense molecule silencing, repressing, or reducing the expression of CSLG in a plant or plant cell has the nucleotide sequence set forth in SEQ ID NO: 108. In some embodiments, a VIGs molecule silencing, repressing, or reducing the expression of CSLG in a plant or plant cell has the nucleotide sequence set forth in SEQ ID NO: 42, SEQ ID NO: 43, SEQ ID NO: 44, SEQ ID NO: 106 or SEQ ID NO: 107. According to certain embodiments, the silencing molecule is targeted to a CSLG fragment having the nucleic acid sequence within the nucleic acid sequence set forth in any one SEQ ID NOS: 125, 127, 30, 32, 34, 36, 38, 40, 65, 80, 93, 95, 97, 99, 101, 103, or 105, or a complementary sequence thereof. In some embodiments, the genetically modified plant comprising said plant cell has a reduced content of a triterpenoid saponin, a metabolite thereof, a derivative thereof, or a biosynthetic intermediate thereof.

According to certain embodiments, the genetically modified or gene edited plant comprise at least one cell comprising at least one silencing molecule targeted to a CSLG gene. According to some embodiments, the at least one silencing molecule is selected from the group consisting of RNA interference molecule and antisense molecule. According to these embodiments, the genetically modified plant comprises reduced content of a steroidal alkaloid or a biosynthetic intermediate thereof, a steroidal saponin or a biosynthetic intermediate thereof, or a triterpenoid saponin or a biosynthetic intermediate thereof.

The silencing molecule target to a CSLG gene can be designed as is known to a person skilled in the art. According to certain embodiments, the silencing molecule comprises a polynucleotide having a nucleic acid sequence substantially complementary to a region of a CSLG gene, for example, but not limited to GAME1S or homologs thereof disclosed herein in tomato, wild tomato, potato, wild potato, and eggplant, or to a complementary sequence of the GAME1S gene. According to certain embodiments, the silencing molecule comprises a polynucleotide having a nucleic acid sequence substantially complementary to a region of a CSLG gene, for example, but not limited to SOAP5 or homologs thereof disclosed herein in Chinese licorice, Arabidopsis, red beet, quinoa, alfalfa, soybean, or Lotus japonicus, or to a complementary sequence of the SOAP5 gene.

According to certain embodiments, the silencing molecule is an antisense RNA.

According to certain exemplary embodiments, the silencing molecule is an RNA interference (RNAi) molecule. According to some embodiments, the silencing molecule is a double-stranded (ds)RNA molecule. According to certain embodiments, the first and the second polynucleotides are separated by a spacer. According to some embodiments, the spacer sequence is an intron. According to yet further embodiments, the expression of the first and the second polynucleotides is derived from one promoter. According to other embodiments, expression of the first and the second polynucleotides are derived from two promoters; the promoters can be identical or different.

In some embodiments, the term “RNA interference” or “RNAi” encompasses the silencing or decreasing of gene expression mediated by small double stranded RNAs. It is the process of sequence-specific, post-transcriptional gene silencing in animals and plants, initiated by inhibitory RNA (iRNA) that is homologous in its duplex region to the sequence of the silenced gene. The gene may be endogenous or exogenous to the organism, present integrated into a chromosome or present in a transfection vector that is not integrated into the genome. The expression of the gene is either completely or partially inhibited. RNAi may also be considered to inhibit the function of a target RNA; the function of the target RNA may be complete or partial.

Typically, the term RNAi molecule refers to single- or double-stranded RNA molecules comprising both a sense and antisense sequence. For example, the RNA interference molecule can be a double-stranded polynucleotide molecule comprising self-complementary sense and antisense regions, wherein the antisense region comprises complementarity to a target nucleic acid molecule. Alternatively the RNAi molecule can be a single-stranded hairpin polynucleotide having self-complementary sense and antisense regions, wherein the antisense region comprises complementarity to a target nucleic acid molecule or it can be a circular single-stranded polynucleotide having two or more loop structures and a stem comprising self-complementary sense and antisense regions, wherein the antisense region comprises complementarity to a target nucleic acid molecule, and wherein the circular polynucleotide can be processed either in vivo or in vitro to generate an active molecule capable of mediating RNAi.

Antisense technology is the process in which an antisense RNA or DNA molecule interacts with a target sense DNA or RNA strand. A sense strand is a 5′ to 3′ mRNA molecule or DNA molecule. The complementary strand, or mirror strand, to the sense is called an antisense. When an antisense strand interacts with a sense mRNA strand, the double helix is recognized as foreign to the cell and will be degraded, resulting in reduced or absent protein production. Although DNA is already a double stranded molecule, antisense technology can be applied to it, building a triplex formation.

One skilled in the art would appreciate that the terms “complementary” or “complement thereof” are used herein to encompass the sequences of polynucleotides which is capable of forming Watson & Crick base pairing with another specified polynucleotide throughout the entirety of the complementary region. This term is applied to pairs of polynucleotides based solely upon their sequences and not any particular set of conditions under which the two polynucleotides would actually bind.

Antisense modulation of cell and/or tissue levels of CSLG may be effected by transforming the cell or tissue with at least one antisense compound, including antisense DNA, antisense RNA, a ribozyme, DNAzyme, a locked nucleic acid (LNA), and an aptamer. In some embodiments, the molecules are chemically modified. In other embodiments, the antisense molecule is antisense DNA or an antisense DNA analog.

RNA antisense strands can be either catalytic or non-catalytic. The catalytic antisense strands, also called ribozymes, cleave the RNA molecule at specific sequences. A non-catalytic RNA antisense strand blocks further RNA processing.

Antisense modulation of cells and/or tissue levels of the CSLG gene may be effected by transforming the organism cells or tissues with at least one antisense compound, including antisense DNA, antisense RNA, a ribozyme, DNAzyme, a locked nucleic acid (LNA), a VIGs, and an aptamer. In some embodiments the molecules are chemically modified. In other embodiments, the antisense molecule is antisense DNA or an antisense DNA analog.

RNAi refers to the introduction of homologous double stranded RNA (dsRNA) to target a specific gene product, resulting in post transcriptional silencing of that gene. This phenomenon was first reported in Caenorhabditis elegans by Guo and Kemphues (1995, Cell, 81(4):611-620) and subsequently Fire et al. (1998, Nature 391:806-811) discovered that it is the presence of dsRNA, formed from the annealing of sense and antisense strands present in the in vitro RNA preps, that is responsible for producing the interfering activity

In both plants and animals, RNAi is mediated by RNA-induced silencing complex (RISC), a sequence-specific, multicomponent nuclease that destroys messenger RNAs homologous to the silencing trigger. RISC is known to contain short RNAs (approximately 22 nucleotides) derived from the double-stranded RNA trigger. The short-nucleotide RNA sequences are homologous to the target gene that is being suppressed. Thus, the short-nucleotide sequences appear to serve as guide sequences to instruct a multicomponent nuclease, RISC, to destroy the specific mRNAs.

The dsRNA used to initiate RNAi, may be isolated from native source or produced by known means, e.g., transcribed from DNA. Plasmids and vectors for generating RNAi molecules against target sequence are now readily available from commercial sources.

The dsRNA can be transcribed from the vectors as two separate strands. In other embodiments, the two strands of DNA used to form the dsRNA may belong to the same or two different duplexes in which they each form with a DNA strand of at least partially complementary sequence. When the dsRNA is thus-produced, the DNA sequence to be transcribed is flanked by two promoters, one controlling the transcription of one of the strands, and the other that of the complementary strand. These two promoters may be identical or different. Alternatively, a single promoter can derive the transcription of single-stranded hairpin polynucleotide having self-complementary sense and antisense regions that anneal to produce the dsRNA.

Inhibition is sequence-specific in that nucleotide sequences corresponding to the duplex region of the RNA are targeted for genetic inhibition. RNA molecules containing a nucleotide sequence identical to a portion of the target gene are preferred for inhibition. RNA sequences with insertions, deletions, and single point mutations relative to the target sequence have also been found to be effective for inhibition. Thus, sequence identity may be optimized by sequence comparison and alignment algorithms known in the art (see Gribskov and Devereux, Sequence Analysis Primer, Stockton Press, 1991, and references cited therein) and calculating the percent difference between the nucleotide sequences by, for example, the Smith-Waterman algorithm as implemented in the BESTFIT software program using default parameters (e.g., University of Wisconsin Genetic Computing Group). Greater than 90% sequence identity, or even 100% sequence identity, between the inhibitory RNA and the portion of the target gene is preferred. Alternatively, the duplex region of the RNA may be defined functionally as a nucleotide sequence that is capable of hybridizing with a portion of the target gene transcript. The length of the identical nucleotide sequences may be at least 25, 50, 100, 200, 300 or 400 bases. There is no upper limit on the length of the dsRNA that can be used. For example, the dsRNA can range from about 21 base pairs (bp) of the gene to the full length of the gene or more.

The term “RNA interference” or “RNAi” refers to the silencing or decreasing of gene expression mediated by small double stranded RNAs. It is the process of sequence-specific, post-transcriptional gene silencing in animals and plants, initiated by inhibitory RNA (iRNA) that is homologous in its duplex region to the sequence of the silenced gene. The gene may be endogenous or exogenous to the organism, present integrated into a chromosome or present in a transfection vector that is not integrated into the genome. The expression of the gene is either completely or partially inhibited. RNAi may also be considered to inhibit the function of a target RNA; the function of the target RNA may be complete or partial.

One of ordinary skill in the art would appreciate that the term RNAi molecule refers to single- or double-stranded RNA molecules comprising both a sense and antisense sequence. For example, the RNA interference molecule can be a double-stranded polynucleotide molecule comprising self-complementary sense and antisense regions, wherein the antisense region comprises complementarity to a target nucleic acid molecule. Alternatively the RNAi molecule can be a single-stranded hairpin polynucleotide having self-complementary sense and antisense regions, wherein the antisense region comprises complementarity to a target nucleic acid molecule or it can be a circular single-stranded polynucleotide having two or more loop structures and a stem comprising self-complementary sense and antisense regions, wherein the antisense region comprises complementarity to a target nucleic acid molecule, and wherein the circular polynucleotide can be processed either in vivo or in vitro to generate an active molecule capable of mediating RNAi.

In some embodiments, the use of RNA interference (RNAi) to down regulate the expression of a CSLG gene is to attenuate the level in plants or a cell thereof or part thereof. In both plants and animals, RNAi is mediated by RNA-induced silencing complex (RISC), a sequence-specific, multicomponent nuclease that destroys messenger RNAs homologous to the silencing trigger. RISC is known to contain short RNAs (approximately 22 nucleotides) derived from the double-stranded RNA trigger. The short-nucleotide RNA sequences are homologous to the target gene that is being suppressed. Thus, the short-nucleotide sequences appear to serve as guide sequences to instruct a multicomponent nuclease, RISC, to destroy the specific mRNAs.

The dsRNA used to initiate RNAi, may be isolated from native source or produced by known means, e.g., transcribed from DNA. Plasmids and vectors for generating RNAi molecules against target sequence are now readily available as exemplified herein below.

According to certain exemplary embodiments, the dsRNA is targeted to CSLG.

In some embodiments, the use of RNA interference (RNAi) to down regulate the expression of a CSLG gene is to attenuate the level of in plants or a cell thereof or part thereof. In both plants and animals, RNAi is mediated by RNA-induced silencing complex (RISC), a sequence-specific, multicomponent nuclease that destroys messenger RNAs homologous to the silencing trigger. RISC is known to contain short RNAs (approximately 22 nucleotides) derived from the double-stranded RNA trigger. The short-nucleotide RNA sequences are homologous to the target gene that is being suppressed. Thus, the short-nucleotide sequences appear to serve as guide sequences to instruct a multicomponent nuclease, RISC, to destroy the specific mRNAs.

The dsRNA used to initiate RNAi, may be isolated from native source or produced by known means, e.g., transcribed from DNA. Plasmids and vectors for generating RNAi molecules against target sequence are now readily available as exemplified herein below.

The dsRNA can be transcribed from the vectors as two separate strands. In other embodiments, the two strands of DNA used to form the dsRNA may belong to the same or two different duplexes in which they each form with a DNA strand of at least partially complementary sequence. When the dsRNA is thus-produced, the DNA sequence to be transcribed is flanked by two promoters, one controlling the transcription of one of the strands, and the other that of the complementary strand. These two promoters may be identical or different. Alternatively, a single promoter can derive the transcription of single-stranded hairpin polynucleotide having self-complementary sense and antisense regions that anneal to produce the dsRNA.

Inhibition is sequence-specific in that nucleotide sequences corresponding to the duplex region of the RNA are targeted for genetic inhibition. RNA molecules containing a nucleotide sequence identical to a portion of the target gene are preferred for inhibition. RNA sequences with insertions, deletions, and single point mutations relative to the target sequence have also been found to be effective for inhibition. Thus, sequence identity may optimized by sequence comparison and alignment algorithms known in the art (see Gribskov and Devereux, Sequence Analysis Primer, Stockton Press, 1991, and references cited therein) and calculating the percent difference between the nucleotide sequences by, for example, the Smith-Waterman algorithm as implemented in the BESTFIT software program using default parameters (e.g., University of Wisconsin Genetic Computing Group). Greater than 90% sequence identity, or even 100% sequence identity, between the inhibitory RNA and the portion of the target gene is preferred. Alternatively, the duplex region of the RNA may be defined functionally as a nucleotide sequence that is capable of hybridizing with a portion of the target gene transcript. The length of the identical nucleotide sequences may be at least 25, 50, 100, 200, 300 or 400 bases. There is no upper limit on the length of the dsRNA that can be used. For example, the dsRNA can range from about 21 base pairs (bp) of the gene to the full length of the gene or more.

Another agent capable of down-regulating the expression of CSLG is a Co-Suppression molecule. Co-suppression is a post-transcriptional mechanism where both the transgene and the endogenous gene are silenced.

Another agent capable of down-regulating the expression of CSLG is a DNAzyme molecule, which is capable of specifically cleaving an mRNA transcript or a DNA sequence of the CSLG. DNAzymes are single-stranded polynucleotides that are capable of cleaving both single- and double-stranded target sequences. A general model (the “10-23” model) for the DNAzyme has been proposed. “10-23” DNAzymes have a catalytic domain of 15 deoxyribonucleotides, flanked by two substrate-recognition domains of seven to nine deoxyribonucleotides each. This type of DNAzyme can effectively cleave its substrate RNA at purine:pyrimidine junctions (for review of DNAzymes, see: Khachigian, L. M. (2002) Curr Opin Mol Ther 4, 119-121).

Examples of construction and amplification of synthetic, engineered DNAzymes recognizing single- and double-stranded target cleavage sites are disclosed in U.S. Pat. No. 6,326,174.

The terms “enzymatic nucleic acid molecule” or “enzymatic oligonucleotide” refers to a nucleic acid molecule which has complementarity in a substrate binding region to a specified gene target, and also has an enzymatic activity which is active to specifically cleave target RNA of CSLG, thereby silencing the gene. The complementary regions allow sufficient hybridization of the enzymatic nucleic acid molecule to the target RNA and subsequent cleavage. The term enzymatic nucleic acid is used interchangeably with for example, ribozymes, catalytic RNA, enzymatic RNA, catalytic DNA, aptazyme or aptamer-binding ribozyme, catalytic oligonucleotide, nucleozyme, DNAzyme, RNAenzyme. The specific enzymatic nucleic acid molecules described in the instant application are not limiting and an enzymatic nucleic acid molecule of disclosed herein requires a specific substrate binding site which is complementary to one or more of the target nucleic acid regions, and that it have nucleotide sequences within or surrounding that substrate binding site which impart a nucleic acid cleaving and/or ligation activity to the molecule. U.S. Pat. No. 4,987,071 discloses examples of such molecules.

According to certain embodiments, the CSLG gene or a silencing molecule targeted thereto form part of an expression vector comprising all necessary elements for expression of the gene or its silencing molecule. According to certain embodiments, the expression is controlled by a constitutive promoter. According to certain embodiments, the constitutive promoter is specific to a plant tissue. According to these embodiments, the tissue specific promoter is selected from the group consisting of root, tuber, leaves and fruit specific promoter. Root specific promoters are described, e.g. in Martinez, E. et al. 2003. Curr. Biol. 13:1435-1441. Fruit specific promoters are described among others in Estornell L. H et al. 2009. Plant Biotechnol. J. 7:298-309 and Fernandez A. I. Et al. 2009 Plant Physiol. 151:1729-1740. Tuber specific promoters are described, e.g. in Rocha-Sosa M, et al., 1989. EMBO J. 8:23-29; McKibbin R. S. et al., 2006. Plant Biotechnol J. 4(4):409-18. Leaf specific promoters are described, e.g. in Yutao Yang, Guodong Yang, Shijuan Liu, Xingqi Guo and Chengchao Zheng. Science in China Series C: Life Sciences. 46: 651-660.

According to certain embodiments, the expression vector further comprises regulatory elements at the 3′ non-coding sequence. As used herein, the “3′ non-coding sequences” refer to DNA sequences located downstream of a coding sequence and include polyadenylation recognition sequences and other sequences encoding regulatory signals capable of affecting mRNA processing or gene expression. The polyadenylation signal is usually characterized by affecting the addition of polyadenylic acid tracts to the 3′ end of the mRNA precursor. The use of different 3′ non-coding sequences is exemplified by Ingelbrecht I L et al. (1989. Plant Cell 1:671-680).

Those skilled in the art will appreciate that the various components of the nucleic acid sequences and the transformation vectors described herein, are operatively linked, so as to result in expression of said nucleic acid or nucleic acid fragment. Techniques for operatively linking the components of the constructs and vectors disclosed herein are well known to those skilled in the art. Such techniques include the use of linkers, such as synthetic linkers, for example including one or more restriction enzyme sites.

Detection of a mutated CSLG gene and/or the presence of silencing molecule targeted to the gene and/or over-expression of the genes is performed employing standard methods of molecular genetics, known to a person of ordinary skill in the art.

For measuring the gene(s) or silencing molecule(s) expression, cDNA or mRNA should be obtained from an organ in which the nucleic acid is expressed. The sample may be further processed before the detecting step. For example, the polynucleotides in the cell or tissue sample may be separated from other components of the sample, may be amplified, etc. All samples obtained from an organism, including those subjected to any sort of further processing are considered to be obtained from the organism.

Detection of the gene(s) or the silencing molecule(s) typically requires amplification of the polynucleotides taken from the candidate altered organism. Methods for DNA amplification are known to a person skilled in the art. Most commonly used method for DNA amplification is PCR (polymerase chain reaction; see, for example, PCR Basics: from background to Bench, Springer Verlag, 2000; Eckert et al., 1991. PCR Methods and Applications 1:17). Additional suitable amplification methods include the ligase chain reaction (LCR), transcription amplification and self-sustained sequence replication, and nucleic acid-based sequence amplification (NASBA).

According to certain embodiments, the nucleic acid sequence comprising the CSLG gene or its silencing molecule further comprises a nucleic acid sequence encoding a selectable marker. According to certain embodiments, the selectable marker confers resistance to antibiotic or to an herbicide; in these embodiments the transgenic plants are selected according to their resistance to the antibiotic or herbicide.

In some embodiments, said endogenous CSLG gene is selectively overexpressed and said altered content comprises an increased amount of the at least one triterpenoid saponin, a derivative thereof, a metabolite thereof, or a biosynthetic intermediate thereof compared to the corresponding unmodified plant.

Overexpression of the CSLG gene can be obtained by any method as is known to a person skilled in the art. According to certain embodiments, disclosed herein are genetically modified plants or parts thereof comprising at least one cell comprising at least one transcribable polynucleotide that is over expressed and encodes at least a CSLG enzyme, wherein the genetically modified plant or plant part comprises elevated content of at least one triterpenoid saponin, a derivative thereof, a metabolite thereof, or a biosynthetic intermediate thereof, compared to a corresponding non-genetically modified plant.

According to some embodiments, the polynucleotides disclosed herein are incorporated in a DNA construct enabling their expression in the plant cell. DNA constructs suitable for use in plants are known to a person skilled in the art. According to one embodiment, the DNA construct comprises at least one expression regulating element selected from the group consisting of a promoter, an enhancer, an origin of replication, a transcription termination sequence, a polyadenylation signal and the like.

The DNA constructs disclosed herein are designed according to the results to be achieved.

In crop plants, reduction of toxic steroidal alkaloids or steroidal glycoalkaloids or their derivatives, metabolites, or biosynthetic intermediates thereof is desired in the edible parts of the plant, including for example, but not limited to fruits, seeds, roots, leaves, and tubers. On the other hand, enriching the content of toxic steroidal glycoalkaloids or biosynthetic intermediates in non-edible parts of the plant, including for example, but not limited to non-edible roots and leaves contributes to the resistance of the plant against a broad range of pathogens. In exemplary embodiments, plants overexpressing the steroidal glycoalkaloids can be used for producing them in the pharmaceutical industry.

In crop plants, modulation of steroidal saponins is also desired. A skilled artisan would appreciate that modulation of steroidal saponins may in certain embodiments, produce high-value products from a certain compound, for example but not limited to for food, cosmetics and pharma industries. In some embodiments, modulation of steroidal saponins produces compounds in plants that will help in protection of plants against pathogens, for example but not limited to protection against bacteria, insect, virus, and insects. In some embodiments, modulation of steroidal saponins removes anti-nutritional compounds from plants.

In crop plants, reduction of toxic or bitter or hormone mimicking triterpenoid saponins or biosynthetic intermediates is desired in the edible parts of the plant, including, for example but not limited to, fruit and seeds. On the other hand, enriching the content of sweet tasting triterpenoid saponins or biosynthetic intermediates thereof is beneficial and could be used for producing natural sweeteners. Plants having increase content of triterpenoid saponins such as QS-21 can be used for producing adjuvants for the pharmaceutical industry.

According to yet additional embodiments, disclosed herein is a genetically modified or gene edited plant having enhanced expression of at least a CSLG gene, wherein the genetically modified or gene edited plant has an increased amount of at least one steroidal alkaloid, of at least one steroidal saponin, or of at least one triterpenoid saponin, compared to a corresponding unmodified or unedited plant. According to yet additional embodiments, disclosed herein is a genetically modified or gene edited plant having decreased expression of at least a CSLG gene, wherein the genetically modified or gene edited plant has a decreased amount of at least one steroidal alkaloid, a decreased amount of at least one steroidal saponin, a decreased amount of at least one triterpenoid saponin, compared to a corresponding unmodified or unedited plant.

The disclosure herein, and in the Examples below, show that by providing at least one heterologous gene encoding an enzyme or other protein involved in the biosynthetic pathway of a steroidal alkaloid, a steroidal saponin, and/or a triterpenoid saponin, for example but not limited to a heterologous CSLG, or by modifying an endogenous gene of the triterpenoid synthetic pathway, for example but not limited to a CSLG gene wherein said modification alters the expression thereof, or the activity or stability of the encoded enzyme, the level of at least one steroidal alkaloid, a derivative thereof, a metabolite thereof, or a biosynthetic intermediate thereof, at least one steroidal saponin, a derivative thereof, a metabolite thereof, or a biosynthetic intermediate thereof, at least one triterpenoid saponin, a derivative thereof, a metabolite thereof, or a biosynthetic intermediate thereof can be altered.

In some embodiments, derivatives of steroidal alkaloids, steroidal saponins, or triterpenoid saponins comprise glycosylated derivatives of, respectively, steroidal alkaloids, steroidal saponins, or triterpenoid saponins.

In some embodiments the amount of the at least one steroidal alkaloid, a derivative thereof, a metabolite thereof, or a biosynthetic intermediate thereof, at least one steroidal saponin, a derivative thereof, a metabolite thereof, or a biosynthetic intermediate thereof, at least one triterpenoid saponin, a derivative thereof, a metabolite thereof, or a biosynthetic intermediate thereof is altered in a plant cell. In some embodiments the amount of the at least one steroidal alkaloid, a derivative thereof, a metabolite thereof, or a biosynthetic intermediate thereof, at least one steroidal saponin, a derivative thereof, a metabolite thereof, or a biosynthetic intermediate thereof, at least one triterpenoid saponin, a derivative thereof, a metabolite thereof, or a biosynthetic intermediate thereof is altered in an algal cell. In some embodiments the amount of the at least one steroidal alkaloid, a derivative thereof, a metabolite thereof, or a biosynthetic intermediate thereof, at least one steroidal saponin, a derivative thereof, a metabolite thereof, or a biosynthetic intermediate thereof, at least one triterpenoid saponin, a derivative thereof, a metabolite thereof, or a biosynthetic intermediate thereof is altered in a yeast. In some embodiments the amount of the at least one steroidal alkaloid, a derivative thereof, a metabolite thereof, or a biosynthetic intermediate thereof, at least one steroidal saponin, a derivative thereof, a metabolite thereof, or a biosynthetic intermediate thereof, at least one triterpenoid saponin, a derivative thereof, a metabolite thereof, or a biosynthetic intermediate thereof is altered in an insect cell. In some embodiments the amount of the at least one steroidal alkaloid, a derivative thereof, a metabolite thereof, or a biosynthetic intermediate thereof, at least one steroidal saponin, a derivative thereof, a metabolite thereof, or a biosynthetic intermediate thereof, at least one triterpenoid saponin, a derivative thereof, a metabolite thereof, or a biosynthetic intermediate thereof is altered in a bacterium. In some embodiments the amount of the at least one steroidal alkaloid, a derivative thereof, a metabolite thereof, or a biosynthetic intermediate thereof, at least one steroidal saponin, a derivative thereof, a metabolite thereof, or a biosynthetic intermediate thereof, at least one triterpenoid saponin, a derivative thereof, a metabolite thereof, or a biosynthetic intermediate thereof is altered in a plant. In some embodiments the amount of the at least one steroidal alkaloid, a derivative thereof, a metabolite thereof, or a biosynthetic intermediate thereof, at least one steroidal saponin, a derivative thereof, a metabolite thereof, or a biosynthetic intermediate thereof, at least one triterpenoid saponin, a derivative thereof, a metabolite thereof, or a biosynthetic intermediate thereof is altered in a plant part.

In some embodiments, derivatives of steroidal alkaloids, steroidal saponins, or triterpenoid saponins comprise glycosylated derivatives of, respectively, steroidal alkaloids, steroidal saponins, or triterpenoid saponins.

For example, in some embodiments, as exemplified below in the Examples, a genetically modified cell, for example a yeast cell or a plant cell or an algal cell or an insect cell or a bacterium, or a genetically modified plant or part thereof having increased expression of a saponin beta-amyrin synthase, a cytochrome P450, a glycosyltransferase, an acyltransferase, a glucuronosyltransferase, or a CSLG, or a combination thereof, has been shown to have an increased level of a triterpenoid saponin, a derivative thereof, a metabolite thereof, or a biosynthetic intermediate thereof. In other embodiments, as shown in the Examples, reducing or silencing expression of a saponin beta-amyrin synthase, cytochrome P450, glycosyltransferase, acyltransferase, glucuronosyltransferase, or CSLG gene has been shown to result in a reduced level of the corresponding triterpenoid saponin, derivative thereof, metabolite thereof, or biosynthetic intermediate thereof.

### Steroidal Alkaloids, Derivatives Thereof, Metabolites Thereof, and Biosynthetic Intermediates Thereof

In some embodiments, a genetically modified cell disclosed herein comprises an increased content of at least one steroidal alkaloid, a derivative thereof, a metabolite thereof, or a biosynthetic intermediate thereof, of any combination thereof. In some embodiments, a genetically modified plant comprising at least one genetically modified cell disclosed herein comprises an increased content of at least one steroidal alkaloid, a derivative thereof, a metabolite thereof, or a biosynthetic intermediate thereof, of any combination thereof. In some embodiments, an in vitro biosynthetic system produces at least one steroidal alkaloid, a derivative thereof, a metabolite thereof, or a biosynthetic intermediate thereof, of any combination thereof. In some embodiments, a genetically modified plant disclosed herein comprises an increased content of at least one steroidal alkaloid, a derivative thereof, a metabolite thereof, or a biosynthetic intermediate thereof, of any combination thereof. In some embodiments, a genetically modified plant comprising at least one genetically modified cell as disclosed herein, comprises a decreased content of at least one steroidal alkaloid, a derivative thereof, a metabolite thereof, or a biosynthetic intermediate thereof, of any combination thereof. In some embodiments, a genetically modified plant disclosed herein comprising at least one genetically modified cells comprises an increased content of at least one steroidal alkaloid, a derivative thereof, a metabolite thereof, or a biosynthetic intermediate thereof, of any combination thereof, and a decreased content of at least one steroidal alkaloid, a derivative thereof, a metabolite thereof, or a biosynthetic intermediate thereof, of any combination thereof.

In some embodiments, a method of producing a steroidal alkaloid in a genetically modified cell disclosed herein comprises producing an at least one steroidal alkaloid, a derivative thereof, a metabolite thereof, or a biosynthetic intermediate thereof, of any combination thereof. In some embodiments, a method of producing a steroidal alkaloid in a genetically modified plant or plant part comprises producing of at least one steroidal alkaloid, a derivative thereof, a metabolite thereof, or a biosynthetic intermediate thereof, of any combination thereof. In some embodiments, disclosed herein is a method of reducing the content of at least one steroidal alkaloid, a derivative thereof, a metabolite thereof, or a biosynthetic intermediate thereof, of any combination thereof in a plant or plant part comprising an at least one genetically modified cell as described herein. In some embodiments, disclosed herein is a method of increasing the content of at least one steroidal alkaloid, a derivative thereof, a metabolite thereof, or a biosynthetic intermediate thereof, of any combination thereof in a a plant or plant part comprising an at least one genetically modified cell as described herein.

In the steroidal alkaloid biosynthetic pathway, steroidal alkaloids branch off from steroidal saponins after the formation of furostanol-type saponin aglycone (FIG. 1). Further steps involving, at least in part, oxidation, transamination, ring formation, etc., result in biosynthesis of steroidal alkaloids (FIG. 1).

Examples of steroidal glycoalkaloids include, but are not limited to, alpha-tomatine, tomatine, dehydrotomatine, alpha-chaonine, alpha-solanine, alpha-solasonine, alpha-solmargine, or any combination thereof.

In some embodiments, the genetically modified cell comprising said increased content comprises a plant cell, a yeast cell, an algal cell, an insect cell, or a bacterial. In some embodiments, the genetically modified cell comprising said increased content comprises a plant cell. In some embodiments, the genetically modified cell comprising said increased content comprises a yeast cell. In some embodiments, the genetically modified cell comprising said increased content comprises an algal cell. In some embodiments, the genetically modified cell comprising said increased content comprises an insect cell. In some embodiments, the genetically modified cell comprising said increased content comprises a bacterium.

According to certain exemplary embodiments, the downstream steroidal glycoalkaloid is selected from the group consisting of esculeosides or dehydroesculeosides.

In some embodiments, a genetically modified plant disclosed herein comprises an increased content of at least one steroidal alkaloid selected from any one of alpha-tomatine, tomatine, dehydrotomatine, alpha-chaconine, alpha-solanine, alpha-solasonine, alpha-solmargine, or any combination thereof. In some embodiments, a genetically modified plant disclosed herein comprises an increased content of alpha-tomatine. In some embodiments, a genetically modified plant disclosed herein comprises an increased content of tomatine. In some embodiments, a genetically modified plant disclosed herein comprises an increased content of dehydrotomatine. In some embodiments, a genetically modified plant disclosed herein comprises an increased content of alpha-chaconine. In some embodiments, a genetically modified plant disclosed herein comprises an increased content of alpha-solanine. In some embodiments, a genetically modified plant disclosed herein comprises an increased content of alpha-solasonine. In some embodiments, a genetically modified plant disclosed herein comprises an increased content of alpha-solmargine.

In some embodiments, a genetically modified plant disclosed herein comprises a decreased content of at least one steroidal alkaloid selected from any one of selected from any one of alpha-tomatine, tomatine, dehydrotomatine, alpha-chaconine, alpha-solanine, alpha-solasonine, alpha-solmargine, or any combination thereof. In some embodiments, a genetically modified plant disclosed herein comprises an increased content of alpha-tomatine. In some embodiments, a genetically modified plant disclosed herein comprises an increased content of tomatine. In some embodiments, a genetically modified plant disclosed herein comprises an increased content of dehydrotomatine. In some embodiments, a genetically modified plant disclosed herein comprises an increased content of alpha-chaconine. In some embodiments, a genetically modified plant disclosed herein comprises an increased content of alpha-solanine. In some embodiments, a genetically modified plant disclosed herein comprises an increased content of alpha-solasonine. In some embodiments, a genetically modified plant disclosed herein comprises an increased content of alpha-solmargine.

In some embodiments, a genetically modified plant disclosed herein comprises an increased content of at least one steroidal and a decreased content of at least one steroidal alkaloid selected from any one of alpha-tomatine, tomatine, dehydrotomatine, alpha-chaconine, alpha-solanine, alpha-solasonine, alpha-solmargine, or any combination thereof.

In some embodiments, a method of producing a steroidal alkaloid in a genetically modified cell comprises producing at least one steroidal alkaloid selected from any one of alpha-tomatine, tomatine, dehydrotomatine, alpha-chaconine, alpha-solanine, alpha-solasonine, alpha-solmargine, or any combination thereof. In some embodiments, a method of producing a steroidal alkaloid in a genetically modified cell comprises producing alpha-tomatine. In some embodiments, a method of producing a steroidal alkaloid in a genetically modified cell comprises producing tomatine. In some embodiments, a method of producing a steroidal alkaloid in a genetically modified cell comprises producing dehydrotomatine. In some embodiments, a method of producing a steroidal alkaloid in a genetically modified cell comprises producing alpha-chaconine. In some embodiments, a method of producing a steroidal alkaloid in a genetically modified cell comprises producing alpha-solanine. In some embodiments, a method of producing a steroidal alkaloid in a genetically modified cell comprises producing alpha-solasonine. In some embodiments, a method of producing a steroidal alkaloid in a genetically modified cell comprises producing alpha-solmargine.

In some embodiments, the method of producing at least one steroidal alkaloid in a genetically modified cell comprises producing at least one steroidal alkaloid in a plant cell, a yeast cell, an algal cell, an insect cell, or a bacterium. In some embodiments, the method of producing at least one steroidal alkaloid in a genetically modified cell comprises producing at least one steroidal alkaloid in a plant cell. In some embodiments, the method of producing at least one steroidal alkaloid in a genetically modified cell comprises producing at least one steroidal alkaloid in a yeast cell. In some embodiments, the method of producing at least one steroidal alkaloid in a genetically modified cell comprises producing at least one steroidal alkaloid in an algal cell. In some embodiments, the method of producing at least one steroidal alkaloid in a genetically modified cell comprises producing at least one steroidal alkaloid in an insect cell. In some embodiments, the method of producing at least one steroidal alkaloid in a genetically modified cell comprises producing at least one steroidal alkaloid in a bacterium. In some embodiments, the plant cell is comprised in a plant or plant part.

In some embodiments, a method of producing a steroidal alkaloid in a genetically modified plant comprises producing at least one steroidal alkaloid selected from any one of alpha-tomatine, tomatine, dehydrotomatine, alpha-chaconine, alpha-solanine, alpha-solasonine, alpha-solmargine, or any combination thereof. In some embodiments, a method of producing a steroidal alkaloid in a genetically modified plant comprises producing alpha-tomatine. In some embodiments, a method of producing a steroidal alkaloid in a genetically modified plant comprises producing tomatine. In some embodiments, a method of producing a steroidal alkaloid in a genetically modified plant comprises producing dehydrotomatine. In some embodiments, a method of producing a steroidal alkaloid in a genetically modified plant comprises producing alpha-chaconine. In some embodiments, a method of producing a steroidal alkaloid in a genetically modified plant comprises producing alpha-solanine. In some embodiments, a method of producing a steroidal alkaloid in a genetically modified plant comprises producing alpha-solasonine. In some embodiments, a method of producing a steroidal alkaloid in a genetically modified plant comprises producing alpha-solmargine. In some embodiments, a method of producing a steroidal alkaloid in a genetically modified plant comprises producing cholesterol. In some embodiments, a method of producing a steroidal alkaloid in a genetically modified plant comprises producing 22-hydroxycholesterol. In some embodiments, a method of producing a steroidal alkaloid in a genetically modified plant comprises producing 22, 26-dihydroxycholesterol. In some embodiments, a method of producing a steroidal alkaloid in a genetically modified plant comprises producing furostanol-type saponin aglycone. In some embodiments, a method of producing a steroidal alkaloid in a genetically modified plant comprises producing furostanol-26-aldehyde. In some embodiments, a method of producing a steroidal alkaloid in a genetically modified plant comprises producing 26-amino-furostanol. In some embodiments, a method of producing a steroidal alkaloid in a genetically modified plant comprises producing tomatidenol. In some embodiments, a method of producing a steroidal alkaloid in a genetically modified plant comprises producing tomatidine. In some embodiments, a method of producing a steroidal alkaloid in a genetically modified plant comprises producing tomatidine galactoside. In some embodiments, a method of producing a steroidal alkaloid in a genetically modified plant comprises producing gamma-tomatine. In some embodiments, a method of producing a steroidal alkaloid in a genetically modified plant comprises producing beta-1-tomatine.

In some embodiments, a method of producing a steroidal alkaloid in an in vitro translation system comprises producing at least one steroidal alkaloid selected from any one of alpha-tomatine, dehydrotomatine, tomatine, alpha-chaconine, alpha-solanine, alpha-solasonine, alpha-solmargine, or any combination thereof. In some embodiments, a method of producing a steroidal alkaloid in an in vitro translation system comprises producing alpha-tomatine. In some embodiments, a method of producing a steroidal alkaloid in an in vitro translation system comprises producing tomatine. In some embodiments, a method of producing a steroidal alkaloid in an in vitro translation system comprises producing alpha-chaconine. In some embodiments, a method of producing a steroidal alkaloid in an in vitro translation system comprises producing alpha-solanine. In some embodiments, a method of producing a steroidal alkaloid in an in vitro translation system comprises producing alpha-solasonine. In some embodiments, a method of producing a steroidal alkaloid in an in vitro translation system comprises producing alpha-solmargine.

In some embodiments, a method of reducing at least one steroidal alkaloid comprises reducing at least one steroidal alkaloid selected from any one of alpha-tomatine, dehydrotomatine, tomatine, alpha-chaconine, alpha-solanine, alpha-solasonine, alpha-solmargine, or any combination thereof. In some embodiments, a method of reducing the content of at least one steroidal alkaloid comprising alpha-tomatine. In some embodiments, a method of reducing at least one steroidal alkaloid comprises reducing tomatine. In some embodiments, a method of reducing at least one steroidal alkaloid comprises reducing dehydrotomatine. In some embodiments, a method of reducing at least one steroidal alkaloid comprises reducing alpha-chaconine. In some embodiments, a method of reducing at least one steroidal alkaloid comprises reducing alpha-solanine. In some embodiments, a method of reducing at least one steroidal alkaloid comprises reducing alpha-solasonine. In some embodiments, a method of reducing at least one steroidal alkaloid comprises reducing alpha-solmargine.

In some embodiments, a method of increasing at least one steroidal alkaloid comprises increasing at least one steroidal alkaloid selected from any one of of alpha-tomatine, dehydrotomatine, tomatine, alpha-chaconine, alpha-solanine, alpha-solasonine, alpha-solmargine, or any combination thereof. In some embodiments, a method of increasing the content of at least one steroidal alkaloid comprising medicagenic acid alpha-tomatine. In some embodiments, a method of increasing at least one steroidal alkaloid comprises increasing tomatine. In some embodiments, a method of increasing at least one steroidal alkaloid comprises increasing alpha-chaconine. In some embodiments, a method of increasing at least one steroidal alkaloid comprises increasing alpha-solanine. In some embodiments, a method of increasing at least one steroidal alkaloid comprises increasing alpha-solasonine. In some embodiments, a method of increasing at least one steroidal alkaloid comprises increasing alpha-solmargine.

In some embodiments, the content of both at least one steroidal alkaloid and at least one steroidal alkaloid biosynthetic intermediate are altered. In some embodiments, an at least one steroidal alkaloid and an at least one steroidal alkaloid biosynthetic intermediate are increased. In some embodiments, an at least one steroidal alkaloids and an at least one steroidal alkaloid biosynthetic intermediate are decreased. In some embodiments, an at least one steroidal alkaloid is increased and an at least one steroidal alkaloid biosynthetic intermediate is decreased. In some embodiments, an at least one steroidal alkaloid is decreased and an at least one steroidal alkaloid biosynthetic intermediate is increased. In some embodiments, the content of a steroidal alkaloid is altered without measurably altering the content of a steroidal alkaloid intermediate. In some embodiments, the content of a steroidal alkaloid intermediate is altered without measurably altering the content of a steroidal alkaloid.

The term “intermediate” may be used interchangeably in some embodiments with the term “biosynthetic intermediate”, having all the same qualities and meanings.

In some embodiments, a biosynthetic intermediate of a steroidal alkaloid comprises cholesterol, 22-hydroxycholesterol, 22,26-dihydroxycholesterol, furostanol-type saponin aglycone, furostanol-26-aldehyde, 26-amino-furostanol, tomatidenol, tomatidine, tomatidine galactoside, gamma-tomatine, beta-1-tomatine, or any combination thereof (FIG. 1).

In some embodiments, a genetically modified cell disclosed herein comprises an increased content of at least one biosynthetic intermediate of a steroidal alkaloid comprising cholesterol, 22-hydroxycholesterol, 22,26-dihydroxycholesterol, furostanol-type saponin aglycone, furostanol-26-aldehyde, 26-amino-furostanol, tomatidenol, tomatidine, tomatidine galactoside, gamma-tomatine, beta-1-tomatine, or any combination thereof. In some embodiments, a genetically modified cell comprises an increased content of at least one biosynthetic intermediate of a steroidal alkaloid comprising cholesterol. In some embodiments, a genetically modified cell comprises an increased content of at least one biosynthetic intermediate of a steroidal alkaloid comprising 22-hydroxycholesterol. In some embodiments, a genetically modified cell comprises an increased content of at least one biosynthetic intermediate of a steroidal alkaloid comprising 22,26-dihydroxycholesterol. In some embodiments, a genetically modified cell comprises an increased content of at least one biosynthetic intermediate of a steroidal alkaloid comprising furostanol-type saponin aglycone. In some embodiments, a genetically modified cell comprises an increased content of at least one biosynthetic intermediate of a steroidal alkaloid comprising furostonal-26-aldehyde. In some embodiments, a genetically modified cell comprises an increased content of at least one biosynthetic intermediate of a steroidal alkaloid comprising 26-amino-furostanol. In some embodiments, a genetically modified cell comprises an increased content of at least one biosynthetic intermediate of a steroidal alkaloid comprising tomatidenol. In some embodiments, a genetically modified cell comprises an increased content of at least one biosynthetic intermediate of a steroidal alkaloid comprising tomatidine. In some embodiments, a genetically modified cell comprises an increased content of at least one biosynthetic intermediate of a steroidal alkaloid comprising tomatidine galactoside. In some embodiments, a genetically modified cell comprises an increased content of at least one biosynthetic intermediate of a steroidal alkaloid comprising gamma-tomatine. In some embodiments, a genetically modified cell comprises an increased content of at least one biosynthetic intermediate of a steroidal alkaloid comprising beta-1-tomatine.

In some embodiments, a genetically modified plant disclosed herein comprises a decreased content of at least one biosynthetic intermediate of a steroidal alkaloid comprising cholesterol, 22-hydroxycholesterol, 22,26-dihydroxycholesterol, furostanol-type saponin aglycone, furostanol-26-aldehyde, 26-amino-furostanol, tomatidenol, tomatidine, tomatidine galactoside, gamma-tomatine, beta-1-tomatine, or any combination thereof. In some embodiments, a genetically modified plant comprises a decreased content of at least one biosynthetic intermediate of a steroidal alkaloid comprising cholesterol. In some embodiments, a genetically modified plant comprises a decreased content of at least one biosynthetic intermediate of a steroidal alkaloid comprising 22-hydroxysholesterol. In some embodiments, a genetically modified plant comprises a decreased content of at least one biosynthetic intermediate of a steroidal alkaloid comprising 22,26-dihydroxycholesterol. In some embodiments, a genetically modified plant comprises a decreased content of at least one biosynthetic intermediate of a steroidal alkaloid comprising furostanol-type saponin aglycone. In some embodiments, a genetically modified plant comprises a decreased content of at least one biosynthetic intermediate of a steroidal alkaloid comprising furostanol-26-aldehyde. In some embodiments, a genetically modified plant comprises a decreased content of at least one biosynthetic intermediate of a steroidal alkaloid comprising 26-amino-furstanol. In some embodiments, a genetically modified plant comprises a decreased content of at least one biosynthetic intermediate of a steroidal alkaloid comprising tomatidenol. In some embodiments, a genetically modified plant comprises a decreased content of at least one biosynthetic intermediate of a steroidal alkaloid comprising tomatidine. In some embodiments, a genetically modified plant comprises a decreased content of at least one biosynthetic intermediate of a steroidal alkaloid comprising tomatidine galactoside. In some embodiments, a genetically modified plant comprises a decreased content of at least one biosynthetic intermediate of a steroidal alkaloid comprising gamma-tomatine. In some embodiments, a genetically modified plant comprises a decreased content of at least one biosynthetic intermediate of a steroidal alkaloid comprising beta-1-tomatine.

In some embodiments, a genetically modified plant disclosed herein comprises an increased content of at least one steroidal alkaloid biosynthetic intermediate and a decreased content of at least one steroidal alkaloid biosynthetic intermediate, said intermediate selected from any one of cholesterol, 22-hydroxycholesterol, 22,26-dihydroxycholesterol, furostanol-type saponin aglycone, furostanol-26-aldehyde, 26-amino-furostanol, tomatidenol, tomatidine, tomatidine galactoside, gamma-tomatine, beta-1-tomatine, or any combination thereof.

In some embodiments, a method of reducing at least one steroidal alkaloid comprises reducing at least one steroidal alkaloid biosynthetic intermediate selected from any one of cholesterol, 22-hydroxycholesterol, 22,26-dihydroxycholesterol, furostanol-type saponin aglycone, furostanol-26-aldehyde, 26-amino-furostanol, tomatidenol, tomatidine, tomatidine galactoside, gamma-tomatine, beta-1-tomatine, or any combination thereof. In some embodiments, a method of reducing at least one steroidal alkaloid comprises reducing at least cholesterol. In some embodiments, a method of reducing at least one steroidal alkaloid comprises reducing at least 22-hydroxycholesterol. In some embodiments, a method of reducing at least one steroidal alkaloid comprises reducing at least 22,26-dihydroxycholesterol. In some embodiments, a method of reducing at least one steroidal alkaloid comprises reducing at least furostanol-type saponin aglycone. In some embodiments, a method of reducing at least one steroidal alkaloid comprises reducing at least furostonal-26-aldehyde. In some embodiments, a method of reducing at least one steroidal alkaloid comprises reducing at least 26-amino-furostanol. In some embodiments, a method of reducing at least one steroidal alkaloid comprises reducing at least tomatidenol. In some embodiments, a method of reducing at least one steroidal alkaloid comprises reducing at tomatidine. In some embodiments, a method of reducing at least one steroidal alkaloid comprises reducing at least tomatidine galactoside. In some embodiments, a method of reducing at least one steroidal alkaloid comprises reducing at least gamma-tomatine. In some embodiments, a method of reducing at least one steroidal alkaloid comprises reducing at least beta-1-tomaine.

In some embodiments, a method of increasing at least one steroidal alkaloid comprises increasing at least one steroidal alkaloid biosynthetic intermediate selected from any one of cholesterol, 22-hydroxycholesterol, 22,26-dihydroxycholesterol, furostanol-type saponin aglycone, furostanol-26-aldehyde, 26-amino-furostanol, tomatidenol, tomatidine, tomatidine galactoside, gamma-tomatine, beta-1-tomatine, or any combination thereof. In some embodiments, a method of increasing at least one steroidal alkaloid comprises increasing at least cholesterol. In some embodiments, a method of increasing at least one steroidal alkaloid comprises increasing at least 22-hydroxycholesterol. In some embodiments, a method of increasing at least one steroidal alkaloid comprises increasing at least 22,26-dihydroxycholesterol. In some embodiments, a method of increasing at least one steroidal alkaloid comprises increasing at least furostanol-type saponin aglycone. In some embodiments, a method of increasing at least one steroidal alkaloid comprises increasing at least furostanol-26-aldehyde. In some embodiments, a method of increasing at least one steroidal alkaloid comprises increasing at least 26-amino-furostanol. In some embodiments, a method of increasing at least one steroidal alkaloid comprises increasing at least tomatidenol. In some embodiments, a method of increasing at least one steroidal alkaloid comprises increasing at least tomatidine. In some embodiments, a method of increasing at least one steroidal alkaloid comprises increasing at least tomatidine galactoside. In some embodiments, a method of increasing at least one steroidal alkaloid comprises increasing at least gamma-tomatine. In some embodiments, a method of increasing at least one steroidal alkaloid comprises increasing at least beta-1-tomatine.

In some embodiments, steroidal alkaloids biosynthetic intermediate comprises any cholesterol, 22-hydroxycholesterol, 22,26-dihydroxycholesterol, furostanol-type saponin aglycone, furostanol-26-aldehyde, 26-amino-furostanol, tomatidenol, tomatidine, tomatidine galactoside, gamma-tomatine, beta-1-tomatine, or a combination thereof.

Unexpectedly, the present disclosure now shows that levels of steroidal alkaloids or their derivatives, metabolites, or biosynthetic intermediates can be increased in cells, for example plant cells or yeast cells or algal cells, or insect cells, or bacterium, or plants by genetically modifying the cell or plant to express at least one heterologous gene encoding an enzyme, for example an enzyme or enzymes of the steroidal alkaloid biosynthetic pathway. In other embodiments, described and exemplified herein are methods of genetically modifying an at least one endogenous gene in a plant cell, for example but not limited to a CSLG gene, to regulate expression, activity, or stability, or any combination thereof. The Examples below disclose enzyme activities and enzymes previously unknown to be part of the steroidal alkaloid biosynthetic pathway. Without this knowledge, production of the steroidal alkaloid compounds was not possible.

In some embodiments, the steroidal alkaloid metabolic pathway can result in cells or plants comprising elevated content of steroidal alkaloids, a derivative thereof, a metabolite thereof, or a biosynthetic intermediate thereof, or in plants having an increased content of these compounds in the plant or plant parts. In some embodiments, plants including but not limited to crop plants are produced, wherein the crop has an increased content of a useful steroidal alkaloid or steroidal alkaloids. In some embodiments, disclosed herein are the means and methods for producing cells including but not limited to plant cells, yeast, or algal cells; plants including but not limited to crop plants, or a part of a plant; having increased levels of a steroidal alkaloid, or steroidal alkaloids, or derivatives thereof, or metabolites thereof, or a biosynthetic intermediate thereof. Alternatively, or additionally, controlling the expression of genes disclosed herein may be used for the production of desired steroidal alkaloids for further use, for example in the pharmaceutical industry or for the formulation of dietary or other supplements, for example but not limited to sweeteners. In some embodiments, these high value saponins may be purified and used, e.g., as sweeteners, foaming agents, emulsifiers, preservatives, anti-carcinogens, hypocholesterolemic agents, anti-inflammatory agents, anti-oxidants, biological adjuvants, anti-microbial agents, insecticidal agents, anti-feedants, or anti-fungal agents, or any combination thereof. The cells and plants disclosed herein comprise compounds of significant nutritional, pharmaceutical, and commercial value.

In some embodiments, derivatives of steroidal alkaloids comprise glycosylated derivatives of steroidal alkaloids.

In some embodiments, a genetically modified cell or genetically modified plant or plant part comprising at least one genetically modified plant cell, as described herein, comprises an altered content of at least a steroidal alkaloid, a derivative thereof, a metabolite thereof, or a biosynthetic intermediate thereof, compared to a corresponding unmodified cell or unmodified plant. In some embodiments, an altered content comprises an increased content. In some embodiments, for example, the genetically modified cell or genetically modified plant has an increased content of at least a steroidal alkaloid, a derivative thereof, a metabolite thereof, or a biosynthetic intermediate thereof, or any combination thereof.

In some embodiments, the genetically modified cell or genetically modified plant or plant part comprising at least one genetically modified plant cell, has an increased content of at least one steroidal alkaloid. In some embodiments, the genetically modified cell or genetically modified plant or plant part comprising at least one genetically modified plant cell, has an increased content of at least two steroidal alkaloids. In some embodiments, the genetically modified cell or genetically modified plant or plant part comprising at least one genetically modified plant cell, has an increased content of at least three steroidal alkaloids. In some embodiments, the genetically modified cell or genetically modified plant or plant part comprising at least one genetically modified plant cell, has an increased content of at least four steroidal alkaloids. In some embodiments, the genetically modified cell or genetically modified plant or plant part comprising at least one genetically modified plant cell, has an increased content of at least five steroidal alkaloids. In some embodiments, the genetically modified cell or genetically modified plant or plant part comprising at least one genetically modified plant cell, has an increased content of at least six steroidal alkaloids.

In some embodiments, the genetically modified cell or genetically modified plant or plant part comprising at least one genetically modified plant cell, has an increased content of at least a derivative of a steroidal alkaloid. In some embodiments, the genetically modified cell or genetically modified plant or plant part comprising at least one genetically modified plant cell, has an increased content of at least two derivatives of a steroidal alkaloid or of steroidal alkaloids. In some embodiments, the genetically modified cell or genetically modified plant or plant part comprising at least one genetically modified plant cell, has an increased content of at least three derivatives of a steroidal alkaloid or of steroidal alkaloids. In some embodiments, the genetically modified cell or genetically modified plant or plant part comprising at least one genetically modified plant cell, has an increased content of at least four derivatives of a steroidal alkaloid or of steroidal alkaloids. In some embodiments, the genetically modified cell or genetically modified plant or plant part comprising at least one genetically modified plant cell, has an increased content of at least five derivatives of a steroidal alkaloid or of steroidal alkaloids. In some embodiments, the genetically modified cell or genetically modified plant or plant part comprising at least one genetically modified plant cell, has an increased content of at least six derivatives of a steroidal alkaloid or of steroidal alkaloids.

In some embodiments, the genetically modified cell or genetically modified plant or plant part comprising at least one genetically modified plant cell, has an increased content of at least a metabolite of a steroidal alkaloid. In some embodiments, the genetically modified cell or genetically modified plant or plant part comprising at least one genetically modified plant cell, has an increased content of at least two metabolites of a steroidal alkaloid or of steroidal alkaloids. In some embodiments, the genetically modified cell or genetically modified plant or plant part comprising at least one genetically modified plant cell, has an increased content of at least three metabolites of a steroidal alkaloid or of steroidal alkaloids. In some embodiments, the genetically modified cell or genetically modified plant or plant part comprising at least one genetically modified plant cell, has an increased content of at least four metabolites of a steroidal alkaloid or of steroidal alkaloids. In some embodiments, the genetically modified cell or genetically modified plant or plant part comprising at least one genetically modified plant cell, has an increased content of at least five metabolites of a steroidal alkaloid or of steroidal alkaloids. In some embodiments, the genetically modified cell or genetically modified plant or plant part comprising at least one genetically modified plant cell, has an increased content of at least six metabolites of a steroidal alkaloid or of steroidal alkaloids.

In some embodiments, the genetically modified cell or genetically modified plant or plant part comprising at least one genetically modified plant cell, has an increased content of at least a biosynthetic intermediate of a steroidal alkaloid. In some embodiments, the genetically modified cell or genetically modified plant or plant part comprising at least one genetically modified plant cell, has an increased content of at least two biosynthetic intermediates of a steroidal alkaloid or of steroidal alkaloids. In some embodiments, the genetically modified cell or genetically modified plant or plant part comprising at least one genetically modified plant cell, has an increased content of at least three biosynthetic intermediates of a steroidal alkaloid or of steroidal alkaloids. In some embodiments, the genetically modified cell or genetically modified plant or plant part comprising at least one genetically modified plant cell, has an increased content of at least four biosynthetic intermediates of a steroidal alkaloid or of steroidal alkaloids. In some embodiments, the genetically modified cell or genetically modified plant or plant part comprising at least one genetically modified plant cell, has an increased content of at least five biosynthetic intermediates of a steroidal alkaloid or of steroidal alkaloids. In some embodiments, the genetically modified cell or genetically modified plant or plant part comprising at least one genetically modified plant cell, has an increased content of at least six biosynthetic intermediates of a steroidal alkaloid or of steroidal alkaloids.

As skilled artisan would recognize that the terms “content” and “level” in reference to a triterpenoid saponin, a derivative thereof, a metabolite thereof, or a biosynthetic intermediate thereof, encompasses the quantity of the compound, for example the quantity of a steroidal alkaloid, a derivative thereof, a metabolite thereof, or a biosynthetic intermediate thereof in a genetically modified cell or in a genetically modified plant, compared with a control cell or control plant. In this context, the terms “content” and “level” may be used interchangeably having all the same meanings and qualities.

In some embodiments, a steroidal alkaloid having increased content comprises a nutrition, cosmetic, or pharmaceutical agent, or any combination thereof. In some embodiments, a steroidal alkaloid having increased content comprises steroidal alkaloid selected from an alpha-tomatine, tomatine, dehydrotomatine, alpha-chaconine, alpha-solanine, alpha-solasonine, alpha-solmargine, or any combination thereof. In some embodiments, a steroidal alkaloid having increased content comprises at least 1, 2, 3, 4, 5, 6, or more steroidal alkaloid selected from alpha-tomatine, tomatine, dehydrotomatine, alpha-chaconine, alpha-solanine, alpha-solasonine, alpha-solmargine, or any combination thereof.

In some embodiments, a steroidal alkaloid having increased content comprises at least 1, 2, 3, 4, 5, 6, or more steroidal alkaloid selected from alpha-tomatine, tomatine, dehydrotomatine, alpha-chaconine, alpha-solanine, alpha-solasonine, alpha-solmargine, or any combination thereof.

In some embodiments, a steroidal alkaloid having increased content in a genetically modified cell, for example a plant cell or a yeast or an algal cell or an insect cell or a bacterium, or in a genetically modified plant or part thereof, comprises steroidal alkaloid selected from alpha-tomatine, tomatine, dehydrotomatine, alpha-chaconine, alpha-solanine, alpha-solasonine, alpha-solmargine, or any combination thereof.

In some embodiments, a genetically modified cell, for example a plant cell or a yeast or an algal cell or an insect cell or a bacterium, or in a genetically modified plant or part thereof, comprises an increased content of alpha-tomatine. In some embodiments, a genetically modified cell, for example a plant cell or a yeast or an algal cell or an insect cell or a bacterium, or in a genetically modified plant or part thereof, comprises an increased content of tomatine. In some embodiments, a genetically modified cell, for example a plant cell or a yeast or an algal cell or an insect cell or a bacterium, or in a genetically modified plant or part thereof, comprises an increased content of dehydrotomatine. In some embodiments, a genetically modified cell, for example a plant cell or a yeast or an algal cell or an insect cell or a bacterium, or in a genetically modified plant or part thereof, comprises an increased content of alpha-chaconine. In some embodiments, a genetically modified cell, for example a plant cell or a yeast or an algal cell or an insect cell or a bacterium, or in a genetically modified plant or part thereof, comprises an increased content of alpha-solanine. In some embodiments, a genetically modified cell, for example a plant cell or a yeast or an algal cell or an insect cell or a bacterium, or in a genetically modified plant or part thereof, comprises an increased content of alpha-solasonine. In some embodiments, a genetically modified cell, for example a plant cell or a yeast or an algal cell or an insect cell or a bacterium, or in a genetically modified plant or part thereof, comprises an increased content of alpha-solmargine. A skilled artisan would appreciate that in some embodiments, instances wherein the content of one steroidal alkaloid is increased additional steroidal alkaloids, or intermediates, or a combination thereof may also be increased in the same time cell.

In some embodiments, a genetically modified cell, for example a plant cell or a yeast or an algal cell, or a genetically modified plant or part thereof, comprises an increased content of a biosynthetic intermediate of a steroidal alkaloid, said intermediate comprising cholesterol, 22-hydroxycholesterol, 22,26-dihydroxycholesterol, furostanol-type-saponin aglycone, furostanol-26-aldehyde, 26-amino-furostanol, tomatidenol, tomatidine, tomatidine galactoside, gamma-tomatine, beta-1-tomatine, or any combination thereof. In some embodiments, a genetically modified cell, for example a plant cell or a yeast or an algal cell or an insect cell or a bacterium, or a genetically modified plant or part thereof, comprises an increased content of at least two biosynthetic intermediates of a steroidal alkaloid or of steroidal alkaloids, said intermediates comprising cholesterol, 22-hydroxycholesterol, 22,26-dihydroxycholesterol, furostanol-type-saponin aglycone, furostanol-26-aldehyde, 26-amino-furostanol, tomatidenol, tomatidine, tomatidine galactoside, gamma-tomatine, beta-1-tomatine, or any combination thereof.

In some embodiments, a genetically modified cell, for example a plant cell or a yeast or an algal cell or an insect cell or a bacterium, or a genetically modified plant or part thereof, comprises an increased content of a biosynthetic intermediate of a steroidal alkaloid, said intermediate comprising cholesterol. In some embodiments, a genetically modified cell, for example a plant cell or a yeast or an algal cell or an insect cell or a bacterium, or a genetically modified plant or part thereof, comprises an increased content of a biosynthetic intermediate of a steroidal alkaloid, said intermediate comprising 22-hydroxycholesteerol. In some embodiments, a genetically modified cell, for example a plant cell or a yeast or an algal cell or an insect cell or a bacterium, or a genetically modified plant or part thereof, comprises an increased content of a biosynthetic intermediate of a steroidal alkaloid, said intermediate comprising 22,26-dihydroxysholesterol. In some embodiments, a genetically modified cell, for example a plant cell or a yeast or an algal cell or an insect cell or a bacterium, or a genetically modified plant or part thereof, comprises an increased content of a biosynthetic intermediate of a steroidal alkaloid, said intermediate comprising furostanol-type saponin aglycone. In some embodiments, a genetically modified cell, for example a plant cell or a yeast or an algal cell or an insect cell or a bacterium, or a genetically modified plant or part thereof, comprises an increased content of a biosynthetic intermediate of a steroidal alkaloid, said intermediate comprising furostanol-26-aldehyde. In some embodiments, a genetically modified cell, for example a plant cell or a yeast or an algal cell or an insect cell or a bacterium, or a genetically modified plant or part thereof, comprises an increased content of a biosynthetic intermediate of a steroidal alkaloid, said intermediate comprising 26-amino-furostanol, or any combination thereof. In some embodiments, a genetically modified cell, for example a plant cell or a yeast or an algal cell or an insect cell or a bacterium, or a genetically modified plant or part thereof, comprises an increased content of a biosynthetic intermediate of a steroidal alkaloid, said intermediate comprising tomatidenol. In some embodiments, a genetically modified cell, for example a plant cell or a yeast or an algal cell or an insect cell or a bacterium, or a genetically modified plant or part thereof, comprises an increased content of a biosynthetic intermediate of a steroidal alkyloid, said intermediate comprising tomatidine. In some embodiments, a genetically modified cell, for example a plant cell or a yeast or an algal cell or an insect cell or a bacterium, or a genetically modified plant or part thereof, comprises an increased content of a biosynthetic intermediate of a steroidal alkaloid, said intermediate comprising tomatidine galactoside, or any combination thereof. In some embodiments, a genetically modified cell, for example a plant cell or a yeast or an algal cell or an insect cell or a bacterium, or a genetically modified plant or part thereof, comprises an increased content of a biosynthetic intermediate of a steroidal alkaloid, said intermediate comprising gamma-tomatine, or any combination thereof. In some embodiments, a genetically modified cell, for example a plant cell or a yeast or an algal cell or an insect cell or a bacterium, or a genetically modified plant or part thereof, comprises an increased content of a biosynthetic intermediate of a steroidal alkaloid, said intermediate comprising beta-1-tomatine, or any combination thereof.

In some embodiments, a steroidal alkaloid having decreased content in a plant or plant part comprising at least one genetically modified cell, comprises a compound having a bitter taste or a toxin. In some embodiments, a steroidal alkaloid having decreased content comprises a steroidal alkaloid selected from alpha-tomatine, tomatine, dehydrotomatine, alpha-chaconine, alpha-solanine, alpha-solasonine, alpha-solmargine, or any combination thereof. In some embodiments, a steroidal alkaloid having decreased content comprises at least 1, 2, 3, 4, 5, 6, or more steroidal alkaloids selected from In some embodiments, a steroidal saponin having increased content in a genetically modified cell, for example a plant cell or a yeast or an algal cell or an insect cell or a bacterium, or in a genetically modified plant or part thereof, comprises a steroidal alkaloid selected from alpha-tomatine, tomatine, dehydrotomatine, alpha-chaconine, alpha-solanine, alpha-solasonine, alpha-solmargine, or any combination thereof.

In some embodiments, a steroidal alkaloid having decreased content comprises at least 1, 2, 3, 4, 5, 6, or more steroidal alkaloids selected from In some embodiments, a steroidal alkaloid having increased content in a genetically modified cell, for example a plant cell or a yeast or an algal cell or an insect cell or a bacterium, or in a genetically modified plant or part thereof, comprises a steroidal alkaloid selected from alpha-tomatine, tomatine, dehydrotomatine, alpha-chaconine, alpha-solanine, alpha-solasonine, alpha-solmargine, or any combination thereof.

In some embodiments, a steroidal alkaloid having decreased content in a genetically modified plant or part thereof comprising at least one genetically modified cell, comprises a steroidal alkaloid selected from alpha-tomatine, tomatine, dehydrotomatine, alpha-chaconine, alpha-solanine, alpha-solasonine, alpha-solmargine, or any combination thereof.

In some embodiments, a genetically modified plant or part thereof comprising at least one genetically modified plant cell, comprises a decreased content of alpha-tomatine. In some embodiments, a genetically modified plant or part thereof comprising at least one genetically modified plant cell, comprises a decreased content of tomatine. In some embodiments, a genetically modified plant or part thereof comprising at least one genetically modified plant cell, comprises a decreased content of dehydrotomatine. In some embodiments, a genetically modified plant or part thereof comprising at least one genetically modified plant cell, comprises a decreased content of alpha-chaconine. In some embodiments, a genetically modified plant or part thereof comprising at least one genetically modified plant cell, comprises a decreased content of alpha-solanine. In some embodiments, a genetically modified plant or part thereof comprising at least one genetically modified plant cell, comprises a decreased content of alpha-solasonine.

In some embodiments, a genetically modified plant or part thereof comprising at least one genetically modified plant cell, comprises a decreased content of a biosynthetic intermediate of a steroidal alkaloid, said intermediate comprising cholesterol, 22-hydroxycholesterol, 22,26-dihydroxycholesterol, furostanol-type-saponin aglycone, furostanol-26-aldehyde, 26-amino-furostanol, tomatidenol, tomatidine, tomatidine galactoside, gamma-tomatine, beta-1-tomatine, or any combination thereof.

In some embodiments, a genetically modified plant or part thereof comprising at least one genetically modified plant cell, comprises a decreased content of a biosynthetic intermediate of a steroidal alkaloid, said intermediate comprising cholesterol. In some embodiments, a genetically modified plant or part thereof comprising at least one genetically modified plant cell, comprises a decreased content of a biosynthetic intermediate of a steroidal alkaloid, said intermediate comprising 22-hydroxycholesterol. In some embodiments, a genetically modified plant or part thereof comprising at least one genetically modified plant cell, comprises a decreased content of a biosynthetic intermediate of a steroidal alkaloid, said intermediate comprising 22,26-dihydroxycholesterol. In some embodiments, a genetically modified plant or part thereof comprising at least one genetically modified plant cell, comprises a decreased content of a biosynthetic intermediate of a steroidal alkaloid, said intermediate comprising fursotanol-type saponin aglycone. In some embodiments, a genetically modified plant or part thereof comprising at least one genetically modified plant cell, comprises a decreased content of a biosynthetic intermediate of a steroidal alkaloid, said intermediate comprising furostanol-26-aldehyde. In some embodiments, a genetically modified plant or part thereof comprising at least one genetically modified plant cell, comprises a decreased content of a biosynthetic intermediate of a steroidal alkaloid, said intermediate comprising 26-amino-furostanol. In some embodiments, a genetically modified plant or part thereof comprising at least one genetically modified plant cell, comprises a decreased content of a biosynthetic intermediate of a steroidal alkaloid, said intermediate comprising tomatidenol. In some embodiments, a genetically modified plant or part thereof comprising at least one genetically modified plant cell, comprises a decreased content of a biosynthetic intermediate of a steroidal alkaloid, said intermediate comprising tomatidine. In some embodiments, a genetically modified plant or part thereof comprising at least one genetically modified plant cell, comprises a decreased content of a biosynthetic intermediate of a steroidal alkaloid, said intermediate comprising tomatidine galactoside. In some embodiments, a genetically modified plant or part thereof comprising at least one genetically modified plant cell, comprises a decreased content of a biosynthetic intermediate of a steroidal alkaloid, said intermediate comprising gamma-tomatine. In some embodiments, a genetically modified plant or part thereof comprising at least one genetically modified plant cell, comprises a decreased content of a biosynthetic intermediate of a steroidal alkaloid, said intermediate comprising beta-1-tomatine.

In some embodiments, a genetically modified cell disclosed herein comprises an increased content of at least one steroidal alkaloid, a derivative thereof, a metabolite thereof, or a biosynthetic intermediate thereof, and an altered content of a phytosterol, or a derivative thereof, a metabolite thereof, or a biosynthetic intermediate thereof; a cholesterol, or a derivative thereof, a metabolite thereof, or a biosynthetic intermediate thereof, a phytocholesterol, or a derivative thereof, a metabolite thereof, or a biosynthetic intermediate thereof; a cholestenol, or a derivative thereof, a metabolite thereof, or a biosynthetic intermediate thereof; or a phytocholestenol, or a derivative thereof, a metabolite thereof, or a biosynthetic intermediate thereof.

In some embodiments, a genetically modified cell disclosed herein comprises an increased content of at least one steroidal alkaloid, a derivative thereof, a metabolite thereof, or a biosynthetic intermediate thereof, and a reduced content of a phytosterol, or a derivative thereof, a metabolite thereof, or a biosynthetic intermediate thereof, a cholesterol, or a derivative thereof, a metabolite thereof, or a biosynthetic intermediate thereof, a phytocholesterol, or a derivative thereof, a metabolite thereof, or a biosynthetic intermediate thereof; a cholestenol, or a derivative thereof, a metabolite thereof, or a biosynthetic intermediate thereof, or a phytocholestenol, or a derivative thereof, a metabolite thereof, or a biosynthetic intermediate thereof.

In some embodiments, a genetically modified cell disclosed herein comprises a decreased content of at least one steroidal alkaloid, a derivative thereof, a metabolite thereof, or a biosynthetic intermediate thereof, and an altered content of a phytosterol, or a derivative thereof, a metabolite thereof, or a biosynthetic intermediate thereof, a cholesterol, or a derivative thereof, a metabolite thereof, or a biosynthetic intermediate thereof, a phytocholesterol, or a derivative thereof, a metabolite thereof, or a biosynthetic intermediate thereof; a cholestenol, or a derivative thereof, a metabolite thereof, or a biosynthetic intermediate thereof, or a phytocholestenol, or a derivative thereof, a metabolite thereof, or a biosynthetic intermediate thereof.

In some embodiments, a genetically modified cell disclosed herein comprises a decreased content of at least one steroidal alkaloid, a derivative thereof, a metabolite thereof, or a biosynthetic intermediate thereof, and an increased content of a phytosterol, or a derivative thereof, a metabolite thereof, or a biosynthetic intermediate thereof, a cholesterol, or a derivative thereof, a metabolite thereof, or a biosynthetic intermediate thereof, a phytocholesterol, or a derivative thereof, a metabolite thereof, or a biosynthetic intermediate thereof; a cholestenol, or a derivative thereof, a metabolite thereof, or a biosynthetic intermediate thereof, or a phytocholestenol, or a derivative thereof, a metabolite thereof, or a biosynthetic intermediate thereof.

In some embodiments, a genetically modified plant disclosed herein comprises an increased content of at least one steroidal alkaloid, a derivative thereof, a metabolite thereof, or a biosynthetic intermediate thereof, and an altered content of a phytosterol, or a derivative thereof, a metabolite thereof, or a biosynthetic intermediate thereof, a cholesterol, or a derivative thereof, a metabolite thereof, or a biosynthetic intermediate thereof, a phytocholesterol, or a derivative thereof, a metabolite thereof, or a biosynthetic intermediate thereof; a cholestenol, or a derivative thereof, a metabolite thereof, or a biosynthetic intermediate thereof, or a phytocholestenol, or a derivative thereof, a metabolite thereof, or a biosynthetic intermediate thereof.

In some embodiments, a genetically modified plant disclosed herein comprises an increased content of at least one steroidal alkaloid, a derivative thereof, a metabolite thereof, or a biosynthetic intermediate thereof, and a decreased content of a phytosterol, or a derivative thereof, a metabolite thereof, or a biosynthetic intermediate thereof, a cholesterol, or a derivative thereof, a metabolite thereof, or a biosynthetic intermediate thereof, a phytocholesterol, or a derivative thereof, a metabolite thereof, or a biosynthetic intermediate thereof; a cholestenol, or a derivative thereof, a metabolite thereof, or a biosynthetic intermediate thereof, or a phytocholestenol, or a derivative thereof, a metabolite thereof, or a biosynthetic intermediate thereof.

In some embodiments, a genetically modified plant disclosed herein comprises a decreased content of at least one steroidal alkaloid, a derivative thereof, a metabolite thereof, or a biosynthetic intermediate thereof and an altered content of a phytosterol, or a derivative thereof, a metabolite thereof, or a biosynthetic intermediate thereof, a cholesterol, or a derivative thereof, a metabolite thereof, or a biosynthetic intermediate thereof, a phytocholesterol, or a derivative thereof, a metabolite thereof, or a biosynthetic intermediate thereof; a cholestenol, or a derivative thereof, a metabolite thereof, or a biosynthetic intermediate thereof, or a phytocholestenol, or a derivative thereof, a metabolite thereof, or a biosynthetic intermediate thereof.

In some embodiments, a genetically modified plant disclosed herein comprises a decreased content of at least one steroidal alkaloid, a derivative thereof, a metabolite thereof, or a biosynthetic intermediate thereof and an increased content of a phytosterol, or a derivative thereof, a metabolite thereof, or a biosynthetic intermediate thereof, a cholesterol, or a derivative thereof, a metabolite thereof, or a biosynthetic intermediate thereof, a phytocholesterol, or a derivative thereof, a metabolite thereof, or a biosynthetic intermediate thereof; a cholestenol, or a derivative thereof, a metabolite thereof, or a biosynthetic intermediate thereof, or a phytocholestenol, or a derivative thereof, a metabolite thereof, or a biosynthetic intermediate thereof.

In some embodiments, a method of producing at least one steroidal alkaloid, a derivative thereof, a metabolite thereof, or a biosynthetic intermediate thereof, in a genetically modified cell or a genetically modified plant comprises increasing the content of at least one steroidal alkaloid, a derivative thereof, a metabolite thereof, or a biosynthetic intermediate thereof, compared to an unmodified cell or an unmodified plant and altering the content of a phytosterol, or a derivative thereof, a metabolite thereof, or a biosynthetic intermediate thereof, a cholesterol, or a derivative thereof, a metabolite thereof, or a biosynthetic intermediate thereof, a phytocholesterol, or a derivative thereof, a metabolite thereof, or a biosynthetic intermediate thereof; a cholestenol, or a derivative thereof, a metabolite thereof, or a biosynthetic intermediate thereof, or a phytocholestenol, or a derivative thereof, a metabolite thereof, or a biosynthetic intermediate thereof.

In some embodiments, a method of producing at least one steroidal alkaloid, a derivative thereof, a metabolite thereof, or a biosynthetic intermediate thereof, in a genetically modified cell or a genetically modified plant comprises increasing the content of at least one steroidal alkaloid, a derivative thereof, a metabolite thereof, or a biosynthetic intermediate thereof, compared to an unmodified cell or an unmodified plant and decreasing the content of a phytosterol, or a derivative thereof, a metabolite thereof, or a biosynthetic intermediate thereof, a cholesterol, or a derivative thereof, a metabolite thereof, or a biosynthetic intermediate thereof, a phytocholesterol, or a derivative thereof, a metabolite thereof, or a biosynthetic intermediate thereof; a cholestenol, or a derivative thereof, a metabolite thereof, or a biosynthetic intermediate thereof, or a phytocholestenol, or a derivative thereof, a metabolite thereof, or a biosynthetic intermediate thereof.

In some embodiments, a method of reducing the content of at least one steroidal alkaloid, a derivative thereof, a metabolite thereof, or a biosynthetic intermediate thereof, comprises genetically modifying at least one plant cell or at least one cell of a plant or plant part, said genetically modified plant cell comprising a reduced content of at least one steroidal alkaloid, a derivative thereof, a metabolite thereof, or a biosynthetic intermediate thereof, and an altered content of a phytosterol, or a derivative thereof, a metabolite thereof, or a biosynthetic intermediate thereof, a cholesterol, or a derivative thereof, a metabolite thereof, or a biosynthetic intermediate thereof, a phytocholesterol, or a derivative thereof, a metabolite thereof, or a biosynthetic intermediate thereof, a cholestenol, or a derivative thereof, a metabolite thereof, or a biosynthetic intermediate thereof, or a phytocholestenol, or a derivative thereof, a metabolite thereof, or a biosynthetic intermediate thereof.

In some embodiments, a method of reducing the content of at least one steroidal alkaloid, a derivative thereof, a metabolite thereof, or a biosynthetic intermediate thereof, comprises genetically modifying at least one plant cell or at least one cell of a plant or plant part, said genetically modified plant cell comprising a reduced content of at least one steroidal alkaloid, a derivative thereof, a metabolite thereof, or a biosynthetic intermediate thereof, and an increased content of a phytosterol, or a derivative thereof, a metabolite thereof, or a biosynthetic intermediate thereof, a cholesterol, or a derivative thereof, a metabolite thereof, or a biosynthetic intermediate thereof, a phytocholesterol, or a derivative thereof, a metabolite thereof, or a biosynthetic intermediate thereof; a cholestenol, or a derivative thereof, a metabolite thereof, or a biosynthetic intermediate thereof, or a phytocholestenol, or a derivative thereof, a metabolite thereof, or a biosynthetic intermediate thereof.

In some embodiments, a method of increasing the content of at least one steroidal alkaloid, a derivative thereof, a metabolite thereof, or a biosynthetic intermediate thereof, comprises genetically modifying at least one plant cell or at least one cell of a plant or plant part, said genetically modified plant cell comprising an altered content of at least one steroidal alkaloid, a derivative thereof, a metabolite thereof, or a biosynthetic intermediate thereof, and a reduced content of a phytosterol, or a derivative thereof, a metabolite thereof, or a biosynthetic intermediate thereof, a cholesterol, or a derivative thereof, a metabolite thereof, or a biosynthetic intermediate thereof, a phytocholesterol, or a derivative thereof, a metabolite thereof, or a biosynthetic intermediate thereof, a cholestenol, or a derivative thereof, a metabolite thereof, or a biosynthetic intermediate thereof, or a phytocholestenol, or a derivative thereof, a metabolite thereof, or a biosynthetic intermediate thereof.

In some embodiments, a method of increasing the content of at least one steroidal alkaloid, a derivative thereof, a metabolite thereof, or a biosynthetic intermediate thereof, comprises genetically modifying at least one plant cell or at least one cell of a plant or plant part, said genetically modified plant cell comprising an increased content of at least one steroidal alkaloid, a derivative thereof, a metabolite thereof, or a biosynthetic intermediate thereof, and a reduced content of a phytosterol, or a derivative thereof, a metabolite thereof, or a biosynthetic intermediate thereof, a cholesterol, or a derivative thereof, a metabolite thereof, or a biosynthetic intermediate thereof, a phytocholesterol, or a derivative thereof, a metabolite thereof, or a biosynthetic intermediate thereof, a cholestenol, or a derivative thereof, a metabolite thereof, or a biosynthetic intermediate thereof, or a phytocholestenol, or a derivative thereof, a metabolite thereof, or a biosynthetic intermediate thereof.

### Steroidal Saponins, Derivatives Thereof, Metabolites Thereof, and Biosynthetic Intermediates Thereof

In some embodiments, a genetically modified cell disclosed herein comprises an increased content of at least one steroidal saponin, a derivative thereof, a metabolite thereof, or a biosynthetic intermediate thereof, of any combination thereof. In some embodiments, a genetically modified plant comprising at least one genetically modified cell disclosed herein comprises an increased content of at least one steroidal saponin, a derivative thereof, a metabolite thereof, or a biosynthetic intermediate thereof, of any combination thereof. In some embodiments, an in vitro biosynthetic system produces at least one steroidal saponin, a derivative thereof, a metabolite thereof, or a biosynthetic intermediate thereof, of any combination thereof. In some embodiments, a genetically modified plant disclosed herein comprises an increased content of at least one steroidal saponin, a derivative thereof, a metabolite thereof, or a biosynthetic intermediate thereof, of any combination thereof. In some embodiments, a genetically modified plant comprising at least one genetically modified cell as disclosed herein, comprises a decreased content of at least one steroidal saponin, a derivative thereof, a metabolite thereof, or a biosynthetic intermediate thereof, of any combination thereof. In some embodiments, a genetically modified plant disclosed herein comprising at least one genetically modified cells comprises an increased content of at least one steroidal saponin, a derivative thereof, a metabolite thereof, or a biosynthetic intermediate thereof, of any combination thereof, and a decreased content of at least one steroidal saponin, a derivative thereof, a metabolite thereof, or a biosynthetic intermediate thereof, of any combination thereof.

In some embodiments, a method of producing a steroidal alkaloid in a genetically modified cell disclosed herein comprises producing an at least one steroidal saponin, a derivative thereof, a metabolite thereof, or a biosynthetic intermediate thereof, of any combination thereof. In some embodiments, a method of producing a steroidal saponin in a genetically modified plant or plant part comprises producing of at least one steroidal alkaloid, a derivative thereof, a metabolite thereof, or a biosynthetic intermediate thereof, of any combination thereof. In some embodiments, disclosed herein is a method of reducing the content of at least one steroidal saponin, a derivative thereof, a metabolite thereof, or a biosynthetic intermediate thereof, of any combination thereof in a plant or plant part comprising an at least one genetically modified cell as described herein. In some embodiments, disclosed herein is a method of increasing the content of at least one steroidal saponin, a derivative thereof, a metabolite thereof, or a biosynthetic intermediate thereof, of any combination thereof in a a plant or plant part comprising an at least one genetically modified cell as described herein.

Steroidal saponins branch off from steroidal alkaloids after the formation of furostanol-type saponin aglycone (FIG. 1).

The commonly used nomenclature for saponins distinguishes between triterpenoid saponins (also called triterpene saponins) and steroidal saponins, which is based on the structure and biochemical background of their aglycones. Both sapogenin types are thought to derive from 2,3-oxidosqualene, a central metabolite in sterol biosynthesis. In phytosterol anabolism, 2,3-oxidosqualene is mainly cyclized into cycloartenol. “Steroidal sapogenins” are thought to derive from intermediates in the phytosterol pathway downstream of cycloartenol formation.

Steroidal saponins, include, but are not limited to, uttroside B, a tomatoside, or any combination thereof.

In some embodiments, the genetically modified cell comprising said increased content comprises a plant cell, a yeast cell, an algal cell, an insect cell, or a bacterial. In some embodiments, the genetically modified cell comprising said increased content comprises a plant cell. In some embodiments, the genetically modified cell comprising said increased content comprises a yeast cell. In some embodiments, the genetically modified cell comprising said increased content comprises an algal cell. In some embodiments, the genetically modified cell comprising said increased content comprises an insect cell. In some embodiments, the genetically modified cell comprising said increased content comprises a bacterium.

In some embodiments, a genetically modified plant disclosed herein comprises an increased content of at least one steroidal saponin selected from any one of uttroside B, a tomatoside, or any combination thereof. In some embodiments, a genetically modified plant disclosed herein comprises an increased content of uttroside B.

In some embodiments, derivatives of steroidal saponins comprise glycosylated derivatives of steroidal saponins.

In some embodiments, a genetically modified plant disclosed herein comprises a decreased content of at least one steroidal saponin selected from any one of uttroside B, a tomatoside, or any combination thereof. In some embodiments, a genetically modified plant disclosed herein comprises a decreased content of uttroside B.

In some embodiments, a genetically modified plant disclosed herein comprises an increased content of at least one steroidal saponin and a decreased content of at least one steroidal saponin selected from any one of uttroside B, a tomatoside, or any combination thereof.

In some embodiments, a method of producing a steroidal saponin in a genetically modified cell comprises producing at least one steroidal saponin selected from any one of uttroside B, a tomatoside, or any combination thereof. In some embodiments, a method of producing a steroidal saponin in a genetically modified cell comprises producing uttroside B.

In some embodiments, the method of producing at least one steroidal saponin in a genetically modified cell comprises producing at least one steroidal saponins in a plant cell, a yeast cell, an algal cell, an insect cell, or a bacterium. In some embodiments, the method of producing at least one steroidal saponin in a genetically modified cell comprises producing at least one steroidal saponins in a plant cell. In some embodiments, the method of producing at least one steroidal saponin in a genetically modified cell comprises producing at least one steroidal saponins in a yeast cell. In some embodiments, the method of producing at least one steroidal saponin in a genetically modified cell comprises producing at least one steroidal saponins in an algal cell. In some embodiments, the method of producing at least one steroidal saponin in a genetically modified cell comprises producing at least one steroidal saponins in an insect cell. In some embodiments, the method of producing at least one steroidal saponin in a genetically modified cell comprises producing at least one steroidal saponins in a bacterium. In some embodiments, the plant cell is comprised in a plant or plant part.

In some embodiments, a method of producing a steroidal saponin in a genetically modified plant comprises producing at least one steroidal saponin selected from any one of any one of uttroside B, a tomatoside, or any combination thereof. In some embodiments, a method of producing a steroidal saponin in a genetically modified plant comprises producing uttroside B.

In some embodiments, a method of producing a steroidal saponin in an in vitro translation system comprises producing at least one steroidal saponin selected from any one of any one of uttroside B, a tomatoside, or any combination thereof. In some embodiments, a method of producing a steroidal saponin in an in vitro translation system comprises producing uttroside B.

In some embodiments, a method of reducing at least one steroidal saponin comprises reducing at least one steroidal saponin selected from any one of any one of uttroside B, a tomatoside, or any combination thereof. In some embodiments, a method of reducing the content of at least one steroidal saponin comprising uttroside B.

In some embodiments, a method of increasing at least one steroidal saponin comprises increasing at least one steroidal saponin selected from any one of any one of uttroside B, a tomatoside, or any combination thereof. In some embodiments, the at least one steroidal saponin comprises uttroside B. In some embodiments, the at least one steroidal saponin comprises a tomatoside.

In some embodiments, the content of both at least one steroidal saponin and at least one steroidal saponin biosynthetic intermediate are altered. In some embodiments, an at least one steroidal saponin and an at least one steroidal saponin biosynthetic intermediate are increased. In some embodiments, an at least one steroidal saponins and an at least one steroidal saponin biosynthetic intermediate are decreased. In some embodiments, an at least one steroidal saponin is increased and an at least one steroidal saponin biosynthetic intermediate is decreased. In some embodiments, an at least one steroidal saponin is decreased and an at least one steroidal saponin biosynthetic intermediate is increased. In some embodiments, the content of a steroidal saponin is altered without measurably altering the content of a steroidal saponin intermediate. In some embodiments, the content of a steroidal saponin intermediate is altered without measurably altering the content of a steroidal saponin.

The term “intermediate” may be used interchangeably in some embodiments with the term “biosynthetic intermediate”, having all the same qualities and meanings.

In some embodiments, a biosynthetic intermediate of a steroidal saponin comprises cholesterol, 22-hydroxycholesterol, 22,26-dihydroxycholesterol, furostanol-type saponin aglycone, or any combination thereof.

In some embodiments, a genetically modified cell disclosed herein comprises an increased content of at least one biosynthetic intermediate of a steroidal saponin comprising cholesterol, 22-hydroxycholesterol, 22,26-dihydroxycholesterol, furostanol-type saponin aglycone, or any combination thereof. In some embodiments, a genetically modified cell comprises an increased content of at least one biosynthetic intermediate of a steroidal saponin comprising cholesterol. In some embodiments, a genetically modified cell comprises an increased content of at least one biosynthetic intermediate of a steroidal saponin comprising 22-hydroxycholesterol. In some embodiments, a genetically modified cell comprises an increased content of at least one biosynthetic intermediate of a steroidal saponin comprising 22,26-dihydroxycholesterol. In some embodiments, a genetically modified cell comprises an increased content of at least one biosynthetic intermediate of a steroidal saponin comprising furostanol-type-saponin aglycone.

In some embodiments, a genetically modified plant disclosed herein comprises a decreased content of at least one biosynthetic intermediate of a steroidal saponin comprising cholesterol, 22-hydroxycholesterol, 22,26-dihydroxycholesterol, furostanol-type saponin aglycone, or any combination thereof. In some embodiments, a genetically modified plant comprises a decreased content of at least one biosynthetic intermediate of a steroidal saponin comprising cholesterol. In some embodiments, a genetically modified plant comprises a decreased content of at least one biosynthetic intermediate of a steroidal saponin comprising 22-hydroxycholesterol. In some embodiments, a genetically modified plant comprises a decreased content of at least one biosynthetic intermediate of a steroidal saponin comprising 22,26-dihydroxycholeseterol. In some embodiments, a genetically modified plant comprises a decreased content of at least one biosynthetic intermediate of a steroidal saponin comprising furostanol-type saponin aglycone.

In some embodiments, a genetically modified plant disclosed herein comprises an increased content of at least one steroidal saponin biosynthetic intermediate and a decreased content of at least one steroidal saponin biosynthetic intermediate, said intermediate selected from any one of cholesterol, 22-hydroxycholesterol, 22,26-dihydroxycholesterol, furostanol-type saponin aglycone, or any combination thereof.

In some embodiments, a method of reducing at least one steroidal saponin comprises reducing at least one steroidal saponin biosynthetic intermediate selected from any one of cholesterol, 22-hydroxycholesterol, 22,26-dihydroxycholesterol, furostanol-type saponin aglycone, or any combination thereof. In some embodiments, a method of reducing at least one steroidal saponin comprises reducing at least cholesterol. In some embodiments, a method of reducing at least one steroidal saponin comprises reducing at least 22-hydroxycholesterol. In some embodiments, a method of reducing at least one steroidal saponin comprises reducing at least 22,26-dihydroxycholesterol. In some embodiments, a method of reducing at least one steroidal saponin comprises reducing at least furostanol-type saponin aglycone.

In some embodiments, a method of increasing at least one steroidal saponin comprises increasing at least one steroidal saponin biosynthetic intermediate selected from any one of cholesterol, 22-hydroxycholesterol, 22,26-dihydroxycholesterol, furostanol-type saponin aglycone, or any combination thereof. In some embodiments, a method of increasing at least one steroidal saponin comprises increasing at least cholesterol. In some embodiments, a method of increasing at least one steroidal saponin comprises increasing at least 22-hydroxycholesterol. In some embodiments, a method of increasing at least one steroidal saponin comprises increasing at least 22,26-dihydroxycholesterol. In some embodiments, a method of increasing at least one steroidal saponin comprises increasing at least furostanol-type saponin aglycone.

In some embodiments, steroidal saponins biosynthetic intermediate comprises any cholesterol, 22-hydroxycholesterol, 22,26-dihydroxycholesterol, furostanol-type saponin aglycone, or a combination thereof.

Unexpectedly, the present disclosure now shows that levels of steroidal saponins or their derivatives, metabolites, or biosynthetic intermediates can be increased in cells, for example plant cells or yeast cells or algal cells, or insect cells, or bacterium, or plants by genetically modifying the cell or plant to express at least one heterologous gene encoding an enzyme, for example an enzyme or enzymes of the steroidal saponin biosynthetic pathway. In other embodiments, described and exemplified herein are methods of genetically modifying an at least one endogenous gene in a plant cell, for example but not limited to a CSLG gene, to regulate expression, activity, or stability, or any combination thereof. The Examples below disclose enzyme activities and enzymes previously unknown to be part of the steroidal saponin biosynthetic pathway. Without this knowledge, production of the steroidal saponin compounds was not possible.

In some embodiments, the steroidal saponin metabolic pathway can result in cells or plants comprising elevated content of steroidal saponins, a derivative thereof, a metabolite thereof, or a biosynthetic intermediate thereof, or in plants having an increased content of these compounds in the plant or plant parts. In some embodiments, plants including but not limited to crop plants are produced, wherein the crop has an increased content of a useful steroidal saponin or steroidal saponins. In some embodiments, disclosed herein are the means and methods for producing cells including but not limited to plant cells, yeast, or algal cells; plants including but not limited to crop plants, or a part of a plant; having increased levels of a steroidal saponin, or steroidal saponins, or derivatives thereof, or metabolites thereof, or a biosynthetic intermediate thereof. Alternatively, or additionally, controlling the expression of genes disclosed herein may be used for the production of desired steroidal saponins for further use, for example in the pharmaceutical industry or for the formulation of dietary or other supplements, for example but not limited to sweeteners. In some embodiments, these high value saponins may be purified and used, e.g., as sweeteners, foaming agents, emulsifiers, preservatives, anti-carcinogens, hypocholesterolemic agents, anti-inflammatory agents, anti-oxidants, biological adjuvants, anti-microbial agents, insecticidal agents, anti-feedants, or anti-fungal agents, or any combination thereof. The cells and plants disclosed herein comprise compounds of significant nutritional, pharmaceutical, and commercial value.

In some embodiments, a genetically modified cell or genetically modified plant or plant part comprising at least one genetically modified plant cell, as described herein, comprises an altered content of at least a steroidal saponin, a derivative thereof, a metabolite thereof, or a biosynthetic intermediate thereof, compared to a corresponding unmodified cell or unmodified plant. In some embodiments, an altered content comprises an increased content. In some embodiments, for example, the genetically modified cell or genetically modified plant has an increased content of at least a steroidal saponin, a derivative thereof, a metabolite thereof, or a biosynthetic intermediate thereof, or any combination thereof.

In some embodiments, the genetically modified cell or genetically modified plant or plant part comprising at least one genetically modified plant cell, has an increased content of at least one steroidal saponin. In some embodiments, the genetically modified cell or genetically modified plant or plant part comprising at least one genetically modified plant cell, has an increased content of at least two steroidal saponins. In some embodiments, the genetically modified cell or genetically modified plant or plant part comprising at least one genetically modified plant cell, has an increased content of at least three steroidal saponins. In some embodiments, the genetically modified cell or genetically modified plant or plant part comprising at least one genetically modified plant cell, has an increased content of at least four steroidal saponins. In some embodiments, the genetically modified cell or genetically modified plant or plant part comprising at least one genetically modified plant cell, has an increased content of at least five steroidal saponins. In some embodiments, the genetically modified cell or genetically modified plant or plant part comprising at least one genetically modified plant cell, has an increased content of at least six steroidal saponins.

In some embodiments, the genetically modified cell or genetically modified plant or plant part comprising at least one genetically modified plant cell, has an increased content of at least a derivative of a steroidal saponin. In some embodiments, the genetically modified cell or genetically modified plant or plant part comprising at least one genetically modified plant cell, has an increased content of at least two derivatives of a steroidal saponin or of steroidal saponins. In some embodiments, the genetically modified cell or genetically modified plant or plant part comprising at least one genetically modified plant cell, has an increased content of at least three derivatives of a steroidal saponin or of steroidal saponins. In some embodiments, the genetically modified cell or genetically modified plant or plant part comprising at least one genetically modified plant cell, has an increased content of at least four derivatives of a steroidal saponin or of steroidal saponins. In some embodiments, the genetically modified cell or genetically modified plant or plant part comprising at least one genetically modified plant cell, has an increased content of at least five derivatives of a steroidal saponin or of steroidal saponins. In some embodiments, the genetically modified cell or genetically modified plant or plant part comprising at least one genetically modified plant cell, has an increased content of at least six derivatives of a steroidal saponin or of steroidal saponins.

In some embodiments, the genetically modified cell or genetically modified plant or plant part comprising at least one genetically modified plant cell, has an increased content of at least a metabolite of a steroidal saponin. In some embodiments, the genetically modified cell or genetically modified plant or plant part comprising at least one genetically modified plant cell, has an increased content of at least two metabolites of a steroidal saponin or of steroidal saponins. In some embodiments, the genetically modified cell or genetically modified plant or plant part comprising at least one genetically modified plant cell, has an increased content of at least three metabolites of a steroidal saponin or of steroidal saponins. In some embodiments, the genetically modified cell or genetically modified plant or plant part comprising at least one genetically modified plant cell, has an increased content of at least four metabolites of a steroidal saponin or of steroidal saponins. In some embodiments, the genetically modified cell or genetically modified plant or plant part comprising at least one genetically modified plant cell, has an increased content of at least five metabolites of a steroidal saponin or of steroidal saponins. In some embodiments, the genetically modified cell or genetically modified plant or plant part comprising at least one genetically modified plant cell, has an increased content of at least six metabolites of a steroidal saponin or of steroidal saponins.

In some embodiments, the genetically modified cell or genetically modified plant or plant part comprising at least one genetically modified plant cell, has an increased content of at least a biosynthetic intermediate of a steroidal saponin. In some embodiments, the genetically modified cell or genetically modified plant or plant part comprising at least one genetically modified plant cell, has an increased content of at least two biosynthetic intermediates of a steroidal saponin or of steroidal saponins. In some embodiments, the genetically modified cell or genetically modified plant or plant part comprising at least one genetically modified plant cell, has an increased content of at least three biosynthetic intermediates of a steroidal saponin or of steroidal saponins. In some embodiments, the genetically modified cell or genetically modified plant or plant part comprising at least one genetically modified plant cell, has an increased content of at least four biosynthetic intermediates of a steroidal saponin or of steroidal saponins. In some embodiments, the genetically modified cell or genetically modified plant or plant part comprising at least one genetically modified plant cell, has an increased content of at least five biosynthetic intermediates of a steroidal saponin or of steroidal saponins. In some embodiments, the genetically modified cell or genetically modified plant or plant part comprising at least one genetically modified plant cell, has an increased content of at least six biosynthetic intermediates of a steroidal saponin or of steroidal saponins.

As skilled artisan would recognize that the terms “content” and “level” in reference to a triterpenoid saponin, a derivative thereof, a metabolite thereof, or a biosynthetic intermediate thereof, encompasses the quantity of the compound, for example the quantity of a steroidal alkaloid, a derivative thereof, a metabolite thereof, or a biosynthetic intermediate thereof in a genetically modified cell or in a genetically modified plant, compared with a control cell or control plant. In this context, the terms “content” and “level” may be used interchangeably having all the same meanings and qualities.

In some embodiments, a steroidal saponin having increased content comprises a nutrition, cosmetic, or pharmaceutical agent, or any combination thereof. In some embodiments, a steroidal saponin having increased content comprises steroidal saponin selected from an uttroside B, a tomatoside or any combination thereof. In some embodiments, a steroidal alkaloid having increased content comprises at least 1, 2, 3, 4, 5, 6, or more steroidal alkaloid selected from uttroside B, a tomatoside, or any combination thereof.

In some embodiments, a steroidal saponin having increased content comprises at least 1, 2, 3, 4, 5, 6, or more steroidal alkaloid selected from uttroside B, a tomatoside or any combination thereof.

In some embodiments, a steroidal saponin having increased content in a genetically modified cell, for example a plant cell or a yeast or an algal cell or an insect cell or a bacterium, or in a genetically modified plant or part thereof, comprises steroidal saponin selected from uttroside B, a tomatoside or any combination thereof.

In some embodiments, a genetically modified cell, for example a plant cell or a yeast or an algal cell or an insect cell or a bacterium, or in a genetically modified plant or part thereof, comprises an increased content of uttroside B. In some embodiments, a genetically modified cell, for example a plant cell or a yeast or an algal cell or an insect cell or a bacterium, or in a genetically modified plant or part thereof, comprises an increased content of a tomatoside.

In some embodiments, a genetically modified cell, for example a plant cell or a yeast or an algal cell, or a genetically modified plant or part thereof, comprises an increased content of a biosynthetic intermediate of a steroidal steroidal saponin, said intermediate comprising cholesterol, 22-hydroxycholesterol, 22,26-dihydroxycholesterol, furostanol-type-saponin aglycone, or any combination thereof. In some embodiments, a genetically modified cell, for example a plant cell or a yeast or an algal cell or an insect cell or a bacterium, or a genetically modified plant or part thereof, comprises an increased content of at least two biosynthetic intermediates of a steroidal saponin or of steroidal saponins, said intermediates comprising cholesterol, 22-hydroxycholesterol, 22,26-dihydroxycholesterol, furostanol-type-saponin aglycone, or any combination thereof.

In some embodiments, a genetically modified cell, for example a plant cell or a yeast or an algal cell or an insect cell or a bacterium, or a genetically modified plant or part thereof, comprises an increased content of a biosynthetic intermediate of a steroidal saponin, said intermediate comprising cholesterol. In some embodiments, a genetically modified cell, for example a plant cell or a yeast or an algal cell or an insect cell or a bacterium, or a genetically modified plant or part thereof, comprises an increased content of a biosynthetic intermediate of a steroidal saponin, said intermediate comprising 22-hydroxycholesteerol. In some embodiments, a genetically modified cell, for example a plant cell or a yeast or an algal cell or an insect cell or a bacterium, or a genetically modified plant or part thereof, comprises an increased content of a biosynthetic intermediate of a steroidal saponin, said intermediate comprising 22,26-dihydroxysholesterol. In some embodiments, a genetically modified cell, for example a plant cell or a yeast or an algal cell or an insect cell or a bacterium, or a genetically modified plant or part thereof, comprises an increased content of a biosynthetic intermediate of a steroidal saponin, said intermediate comprising furostanol-type saponin aglycone.

In some embodiments, a steroidal saponin having decreased content in a plant or plant part comprising at least one genetically modified cell, comprises a compound having a bitter taste or a toxin. In some embodiments, a steroidal saponin having decreased content comprises a steroidal saponin selected from uttroside B, a tomatoside or any combination thereof. In some embodiments, a steroidal saponin having decreased content comprises at least 1, 2, 3, 4, 5, 6, or more steroidal saponins selected from In some embodiments, a steroidal saponin having increased content in a genetically modified cell, for example a plant cell or a yeast or an algal cell or an insect cell or a bacterium, or in a genetically modified plant or part thereof, comprises steroidal saponin selected from uttroside B, a tomatoside or any combination thereof.

In some embodiments, a steroidal saponin having decreased content comprises at least 1, 2, 3, 4, 5, 6, or more steroidal saponins selected from In some embodiments, a steroidal saponin having increased content in a genetically modified cell, for example a plant cell or a yeast or an algal cell or an insect cell or a bacterium, or in a genetically modified plant or part thereof, comprises a steroidal saponin selected from uttroside B, a tomatoside or any combination thereof.

In some embodiments, a steroidal saponin having decreased content in a genetically modified plant or part thereof comprising at least one genetically modified cell, comprises a steroidal saponin selected from uttroside B, a tomatoside or any combination thereof.

In some embodiments, a genetically modified plant or part thereof comprising at least one genetically modified plant cell, comprises a decreased content of uttroside B. In some embodiments, a genetically modified plant or part thereof comprising at least one genetically modified plant cell, comprises a decreased content of a tomatoside.

In some embodiments, a genetically modified plant or part thereof comprising at least one genetically modified plant cell, comprises a decreased content of a biosynthetic intermediate of a steroidal saponin, said intermediate comprising cholesterol, 22-hydroxycholesterol, 22,26-dihydroxycholesterol, furostanol-type-saponin aglycone, or any combination thereof.

In some embodiments, a genetically modified plant or part thereof comprising at least one genetically modified plant cell, comprises a decreased content of a biosynthetic intermediate of a steroidal saponin, said intermediate comprising cholesterol. In some embodiments, a genetically modified plant or part thereof comprising at least one genetically modified plant cell, comprises a decreased content of a biosynthetic intermediate of a steroidal saponin, said intermediate comprising 22-hydroxycholesterol. In some embodiments, a genetically modified plant or part thereof comprising at least one genetically modified plant cell, comprises a decreased content of a biosynthetic intermediate of a steroidal saponin, said intermediate comprising 22, 26-dihydroxycholesterol. In some embodiments, a genetically modified plant or part thereof comprising at least one genetically modified plant cell, comprises a decreased content of a biosynthetic intermediate of a steroidal saponin, said intermediate comprising furostanol-type saponin aglycone.

In some embodiments, a genetically modified cell disclosed herein comprises an increased content of at least one steroidal saponin, a derivative thereof, a metabolite thereof, or a biosynthetic intermediate thereof, and an altered content of a phytosterol, or a derivative thereof, a metabolite thereof, or a biosynthetic intermediate thereof, a cholesterol, or a derivative thereof, a metabolite thereof, or a biosynthetic intermediate thereof, a phytocholesterol, or a derivative thereof, a metabolite thereof, or a biosynthetic intermediate thereof; a cholestenol, or a derivative thereof, a metabolite thereof, or a biosynthetic intermediate thereof, or a phytocholestenol, or a derivative thereof, a metabolite thereof, or a biosynthetic intermediate thereof.

In some embodiments, a genetically modified cell disclosed herein comprises an increased content of at least one steroidal saponin, a derivative thereof, a metabolite thereof, or a biosynthetic intermediate thereof, and a reduced content of a phytosterol, or a derivative thereof, a metabolite thereof, or a biosynthetic intermediate thereof, a cholesterol, or a derivative thereof, a metabolite thereof, or a biosynthetic intermediate thereof, a phytocholesterol, or a derivative thereof, a metabolite thereof, or a biosynthetic intermediate thereof; a cholestenol, or a derivative thereof, a metabolite thereof, or a biosynthetic intermediate thereof, or a phytocholestenol, or a derivative thereof, a metabolite thereof, or a biosynthetic intermediate thereof.

In some embodiments, a genetically modified cell disclosed herein comprises a decreased content of at least one steroidal saponin, a derivative thereof, a metabolite thereof, or a biosynthetic intermediate thereof, and an altered content of a phytosterol, or a derivative thereof, a metabolite thereof, or a biosynthetic intermediate thereof, a cholesterol, or a derivative thereof, a metabolite thereof, or a biosynthetic intermediate thereof, a phytocholesterol, or a derivative thereof, a metabolite thereof, or a biosynthetic intermediate thereof; a cholestenol, or a derivative thereof, a metabolite thereof, or a biosynthetic intermediate thereof, or a phytocholestenol, or a derivative thereof, a metabolite thereof, or a biosynthetic intermediate thereof.

In some embodiments, a genetically modified cell disclosed herein comprises a decreased content of at least one steroidal saponin, a derivative thereof, a metabolite thereof, or a biosynthetic intermediate thereof, and an increased content of a phytosterol, or a derivative thereof, a metabolite thereof, or a biosynthetic intermediate thereof, a cholesterol, or a derivative thereof, a metabolite thereof, or a biosynthetic intermediate thereof, a phytocholesterol, or a derivative thereof, a metabolite thereof, or a biosynthetic intermediate thereof; a cholestenol, or a derivative thereof, a metabolite thereof, or a biosynthetic intermediate thereof, or a phytocholestenol, or a derivative thereof, a metabolite thereof, or a biosynthetic intermediate thereof.

In some embodiments, a genetically modified plant disclosed herein comprises an increased content of at least one steroidal saponin, a derivative thereof, a metabolite thereof, or a biosynthetic intermediate thereof, and an altered content of a phytosterol, or a derivative thereof, a metabolite thereof, or a biosynthetic intermediate thereof, a cholesterol, or a derivative thereof, a metabolite thereof, or a biosynthetic intermediate thereof, a phytocholesterol, or a derivative thereof, a metabolite thereof, or a biosynthetic intermediate thereof; a cholestenol, or a derivative thereof, a metabolite thereof, or a biosynthetic intermediate thereof, or a phytocholestenol, or a derivative thereof, a metabolite thereof, or a biosynthetic intermediate thereof.

In some embodiments, a genetically modified plant disclosed herein comprises an increased content of at least one steroidal saponin, a derivative thereof, a metabolite thereof, or a biosynthetic intermediate thereof, and a decreased content of a phytosterol, or a derivative thereof, a metabolite thereof, or a biosynthetic intermediate thereof, a cholesterol, or a derivative thereof, a metabolite thereof, or a biosynthetic intermediate thereof, a phytocholesterol, or a derivative thereof, a metabolite thereof, or a biosynthetic intermediate thereof; a cholestenol, or a derivative thereof, a metabolite thereof, or a biosynthetic intermediate thereof, or a phytocholestenol, or a derivative thereof, a metabolite thereof, or a biosynthetic intermediate thereof.

In some embodiments, a genetically modified plant disclosed herein comprises a decreased content of at least one steroidal saponin, a derivative thereof, a metabolite thereof, or a biosynthetic intermediate thereof and an altered content of a phytosterol, or a derivative thereof, a metabolite thereof, or a biosynthetic intermediate thereof, a cholesterol, or a derivative thereof, a metabolite thereof, or a biosynthetic intermediate thereof, a phytocholesterol, or a derivative thereof, a metabolite thereof, or a biosynthetic intermediate thereof; a cholestenol, or a derivative thereof, a metabolite thereof, or a biosynthetic intermediate thereof, or a phytocholestenol, or a derivative thereof, a metabolite thereof, or a biosynthetic intermediate thereof.

In some embodiments, a genetically modified plant disclosed herein comprises a decreased content of at least one steroidal saponin, a derivative thereof, a metabolite thereof, or a biosynthetic intermediate thereof and an increased content of a phytosterol, or a derivative thereof, a metabolite thereof, or a biosynthetic intermediate thereof, a cholesterol, or a derivative thereof, a metabolite thereof, or a biosynthetic intermediate thereof, a phytocholesterol, or a derivative thereof, a metabolite thereof, or a biosynthetic intermediate thereof; a cholestenol, or a derivative thereof, a metabolite thereof, or a biosynthetic intermediate thereof, or a phytocholestenol, or a derivative thereof, a metabolite thereof, or a biosynthetic intermediate thereof.

In some embodiments, a method of producing at least one steroidal saponin, a derivative thereof, a metabolite thereof, or a biosynthetic intermediate thereof, in a genetically modified cell or a genetically modified plant comprises increasing the content of at least one steroidal saponin, a derivative thereof, a metabolite thereof, or a biosynthetic intermediate thereof, compared to an unmodified cell or an unmodified plant and altering the content of a phytosterol, or a derivative thereof, a metabolite thereof, or a biosynthetic intermediate thereof, a cholesterol, or a derivative thereof, a metabolite thereof, or a biosynthetic intermediate thereof, a phytocholesterol, or a derivative thereof, a metabolite thereof, or a biosynthetic intermediate thereof; a cholestenol, or a derivative thereof, a metabolite thereof, or a biosynthetic intermediate thereof, or a phytocholestenol, or a derivative thereof, a metabolite thereof, or a biosynthetic intermediate thereof.

In some embodiments, a method of producing at least one steroidal saponin, a derivative thereof, a metabolite thereof, or a biosynthetic intermediate thereof, in a genetically modified cell or a genetically modified plant comprises increasing the content of at least one steroidal saponin, a derivative thereof, a metabolite thereof, or a biosynthetic intermediate thereof, compared to an unmodified cell or an unmodified plant and decreasing the content of a phytosterol, or a derivative thereof, a metabolite thereof, or a biosynthetic intermediate thereof, a cholesterol, or a derivative thereof, a metabolite thereof, or a biosynthetic intermediate thereof, a phytocholesterol, or a derivative thereof, a metabolite thereof, or a biosynthetic intermediate thereof; a cholestenol, or a derivative thereof, a metabolite thereof, or a biosynthetic intermediate thereof, or a phytocholestenol, or a derivative thereof, a metabolite thereof, or a biosynthetic intermediate thereof.

In some embodiments, a method of reducing the content of at least one steroidal saponin, a derivative thereof, a metabolite thereof, or a biosynthetic intermediate thereof, comprises genetically modifying at least one plant cell or at least one cell of a plant or plant part, said genetically modified plant cell comprising a reduced content of at least one steroidal saponin, a derivative thereof, a metabolite thereof, or a biosynthetic intermediate thereof, and an altered content of a phytosterol, or a derivative thereof, a metabolite thereof, or a biosynthetic intermediate thereof, a cholesterol, or a derivative thereof, a metabolite thereof, or a biosynthetic intermediate thereof, a phytocholesterol, or a derivative thereof, a metabolite thereof, or a biosynthetic intermediate thereof, a cholestenol, or a derivative thereof, a metabolite thereof, or a biosynthetic intermediate thereof, or a phytocholestenol, or a derivative thereof, a metabolite thereof, or a biosynthetic intermediate thereof.

In some embodiments, a method of reducing the content of at least one steroidal saponin, a derivative thereof, a metabolite thereof, or a biosynthetic intermediate thereof, comprises genetically modifying at least one plant cell or at least one cell of a plant or plant part, said genetically modified plant cell comprising a reduced content of at least one steroidal saponin, a derivative thereof, a metabolite thereof, or a biosynthetic intermediate thereof, and an increased content of a phytosterol, or a derivative thereof, a metabolite thereof, or a biosynthetic intermediate thereof; a cholesterol, or a derivative thereof, a metabolite thereof, or a biosynthetic intermediate thereof, a phytocholesterol, or a derivative thereof, a metabolite thereof, or a biosynthetic intermediate thereof, a cholestenol, or a derivative thereof, a metabolite thereof, or a biosynthetic intermediate thereof, or a phytocholestenol, or a derivative thereof, a metabolite thereof, or a biosynthetic intermediate thereof.

In some embodiments, a method of increasing the content of at least one steroidal saponin, a derivative thereof, a metabolite thereof, or a biosynthetic intermediate thereof, comprises genetically modifying at least one plant cell or at least one cell of a plant or plant part, said genetically modified plant cell comprising an altered content of at least one steroidal saponin, a derivative thereof, a metabolite thereof, or a biosynthetic intermediate thereof, and a reduced content of a phytosterol, or a derivative thereof, a metabolite thereof, or a biosynthetic intermediate thereof, a cholesterol, or a derivative thereof, a metabolite thereof, or a biosynthetic intermediate thereof, a phytocholesterol, or a derivative thereof, a metabolite thereof, or a biosynthetic intermediate thereof, a cholestenol, or a derivative thereof, a metabolite thereof, or a biosynthetic intermediate thereof, or a phytocholestenol, or a derivative thereof, a metabolite thereof, or a biosynthetic intermediate thereof.

In some embodiments, a method of increasing the content of at least one steroidal saponin, a derivative thereof, a metabolite thereof, or a biosynthetic intermediate thereof, comprises genetically modifying at least one plant cell or at least one cell of a plant or plant part, said genetically modified plant cell comprising an increased content of at least one steroidal saponin, a derivative thereof, a metabolite thereof, or a biosynthetic intermediate thereof, and a reduced content of a phytosterol, or a derivative thereof, a metabolite thereof, or a biosynthetic intermediate thereof, a cholesterol, or a derivative thereof, a metabolite thereof, or a biosynthetic intermediate thereof, a phytocholesterol, or a derivative thereof, a metabolite thereof, or a biosynthetic intermediate thereof, a cholestenol, or a derivative thereof, a metabolite thereof, or a biosynthetic intermediate thereof, or a phytocholestenol, or a derivative thereof, a metabolite thereof, or a biosynthetic intermediate thereof.

### Triterpenoid Saponins, Derivatives Thereof, Metabolites Thereof, and Biosynthetic Intermediates Thereof

In some embodiments, a genetically modified cell disclosed herein comprises an increased content of at least one triterpenoids saponin, a derivative thereof, a metabolite thereof, or a biosynthetic intermediate thereof, of any combination thereof. In some embodiments, a genetically modified plant comprising at least one genetically modified cell disclosed herein comprises an increased content of at least one triterpenoids saponin, a derivative thereof, a metabolite thereof, or a biosynthetic intermediate thereof, of any combination thereof. In some embodiments, an in vitro biosynthetic system produces at least one triterpenoids saponin, a derivative thereof, a metabolite thereof, or a biosynthetic intermediate thereof, of any combination thereof. In some embodiments, a genetically modified plant disclosed herein comprises an increased content of at least one triterpenoids saponin, a derivative thereof, a metabolite thereof, or a biosynthetic intermediate thereof, of any combination thereof. In some embodiments, a genetically modified plant comprising at least one genetically modified cell as disclosed herein, comprises a decreased content of at least one triterpenoids saponin, a derivative thereof, a metabolite thereof, or a biosynthetic intermediate thereof, of any combination thereof. In some embodiments, a genetically modified plant disclosed herein comprising at least one genetically modified cells comprises an increased content of at least one triterpenoids saponin, a derivative thereof, a metabolite thereof, or a biosynthetic intermediate thereof, of any combination thereof, and a decreased content of at least one triterpenoids saponin, a derivative thereof, a metabolite thereof, or a biosynthetic intermediate thereof, of any combination thereof.

In some embodiments, a method of producing a triterpenoid saponin in a genetically modified cell disclosed herein comprises producing an at least one triterpenoids saponin, a derivative thereof, a metabolite thereof, or a biosynthetic intermediate thereof, of any combination thereof. In some embodiments, a method of producing a triterpenoid saponin in a genetically modified plant or plant part comprises producing of at least one triterpenoids saponin, a derivative thereof, a metabolite thereof, or a biosynthetic intermediate thereof, of any combination thereof. In some embodiments, disclosed herein is a method of reducing the content of at least one triterpenoids saponin, a derivative thereof, a metabolite thereof, or a biosynthetic intermediate thereof, of any combination thereof in a plant or plant part comprising an at least one genetically modified cell as described herein. In some embodiments, disclosed herein is a method of increasing the content of at least one triterpenoids saponin, a derivative thereof, a metabolite thereof, or a biosynthetic intermediate thereof, of any combination thereof in a plant or plant part comprising an at least one genetically modified cell as described herein.

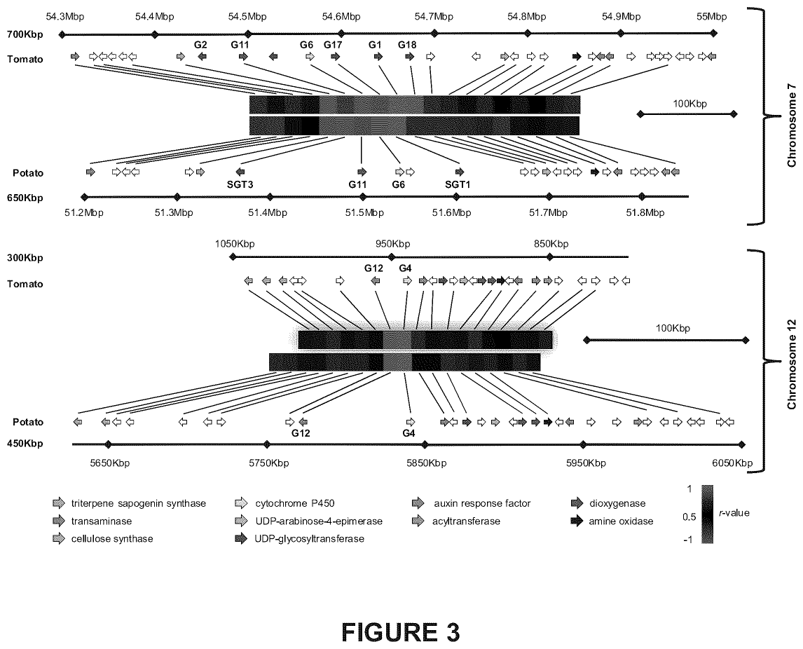

The commonly used nomenclature for saponins distinguishes between triterpenoid saponins (also called triterpene saponins) and steroidal saponins, which is based on the structure and biochemical background of their aglycones. Both sapogenin types are thought to derive from 2,3-oxidosqualene, a central metabolite in sterol biosynthesis. In phytosterol anabolism, 2,3-oxidosqualene is mainly cyclized into cycloartenol. “Triterpenoid sapogenins” branch off the phytosterol pathway by alternative cyclization of 2,3-oxidosqualene, while “steroidal sapogenins” are thought to derive from intermediates in the phytosterol pathway downstream of cycloartenol formation (see FIG. 3A).

Saponins may be selected from the group comprising dammarane-type saponins, tirucallane-type saponins, lupane-type saponins, oleanane-type saponins, taraxasterane-type saponins, ursane-type saponins, hopane-type saponins, cucurbitane-type saponins, cycloartane-type saponins, lanostane-type saponins, and steroid-type saponins. The aglycon backbones, the sapogenins, can be similarly classified and may be selected from the group comprising dammarane-type sapogenins, tirucallane-type sapogenins, lupane-type sapogenins, oleanane-type sapogenins, taraxasterane-type sapogenins, ursane-type sapogenins, hopane-type sapogenins, cucurbitane-type sapogenins, cycloartane-type sapogenins, lanostane-type sapogenins, and steroid-type sapogenins.

In some embodiments, the “triterpenoid sapogenins” disclosed herein may be selected from the group comprising dammarane-type sapogenins, tirucallane-type sapogenins, lupane-type sapogenins, oleanane-type sapogenins, ursane-type sapogenins, and hopane-type sapogenins. In some embodiment, the triterpenoid sapogenins as produced by the method of the disclosure are dammarane-type sapogenins, or tirucallane-type sapogenins, or lupane-type sapogenins, or oleanane-type sapogenins, or ursane-type sapogenins, or hopane-type sapogenins.

Triterpenoid sapogenins typically have a tetracyclic or pentacyclic skeleton. In some embodiments, the sapogenin building blocks themselves may have multiple modifications, for example but not limited to small functional groups, including hydroxyl, keto, aldehyde, and carboxyl moieties, of precursor sapogenin backbones such as βamyrin, lupeol, and dammarenediol.

The terms “triterpene” and “triterpenoid” may be used interchangeably having the same meaning and qualities. Triterpenoid saponins comprise multiple functions and may be used in different roles including but not limited to as a sweetener, a foaming agent, an emulsifier, a preservative, an anti-carcinogen, a hypocholesterolemic agent, an anti-inflammatory agent, an anti-oxidant, a biological adjuvant, an anti-microbial agent, an insecticidal agent, an antifeedant, an anti-fungal agent, or any combination thereof.

In some embodiments, a triterpenoid sapogenins, as disclosed herein, also encompass new-to-nature triterpenoid compounds, which are structurally related to the naturally occurring triterpenoid sapogenins. These new-to-nature triterpenoid sapogenins may be novel compounds that can be obtained after genetic engineering of the synthesizing eukaryotic host cell, for example a plant cell or a yeast cell.

Examples of triterpenoid saponins and biosynthetic intermediates thereof are presented in Table 2 below.

TABLE 2
Triterpenoid Saponins and Biosynthetic Intermediates Thereof

| COMPOUND | STRUCTURE | NAME |
|---|---|---|
| 1 | 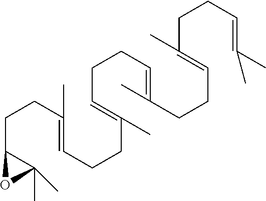 | (S)-2,3- Oxidosqualene |
| 2 | 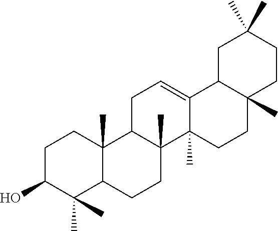 | β-Amyrin |
| 3 | 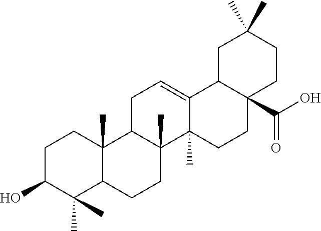 | Oleanolic acid (OA) |
| 4 | 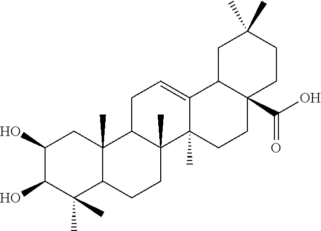 | Augustic acid (AA) |
| 5 | 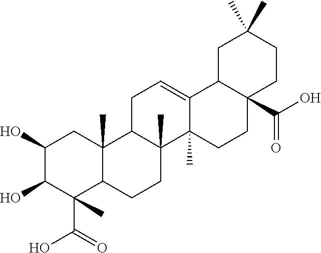 | Medicagenic acid (MA) |
| 6 | 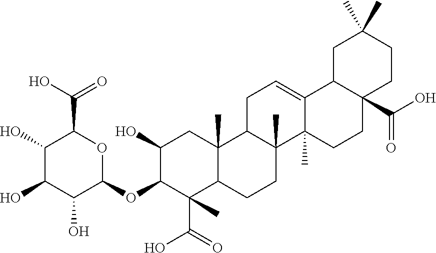 | Medicagenic acid 3-glucuronide |
| 7 | 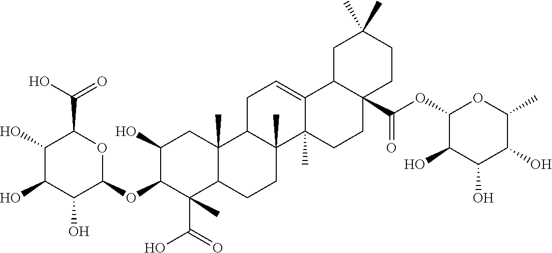 | Yossoside I (3-O-[β-D- glucuronopyranos yl-28-O-β-D- fucopyranosyl- medicagenic acid) |
| 8 | 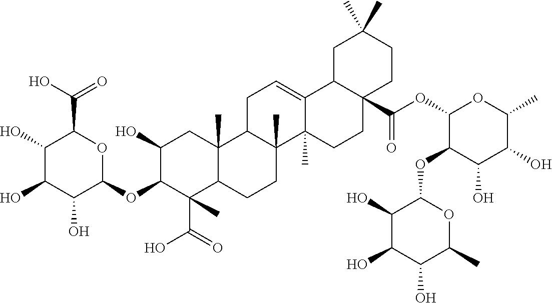 | Yossoside II (3-O-[β-D- glucuronopyranos yl]-28-O-[α-L- rhamnopyranosyl- (1->2)-β-D- fucopyranosyl]- medicagenic acid) |
| 9 | 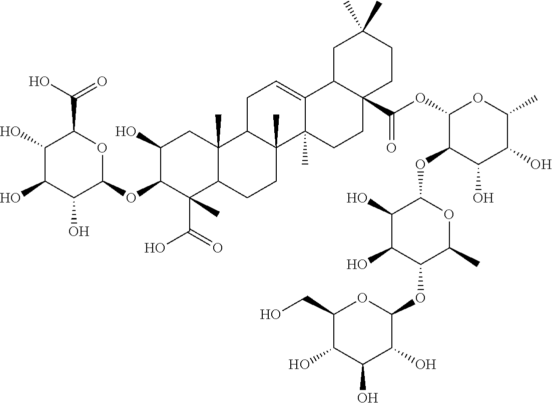 | Yossoside III (3-O-[β-D- glucuronopyranos yl]-28-O-[β-D- glucopyranosyl- (->4)-α-L- rhamnopyranosyl- (1->2)-ß-D- fucopyranosyl]- medicagenic acid) |
| 10 | 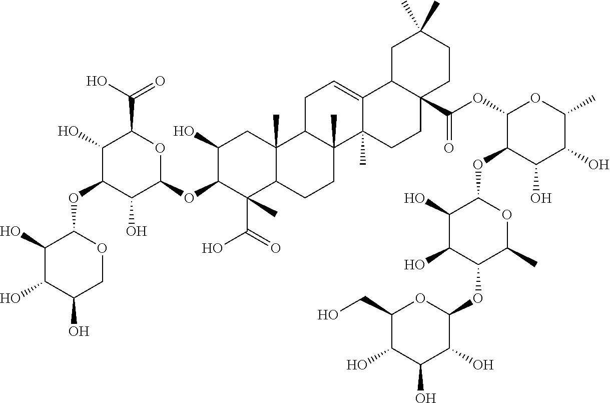 | Yossoside IV (3-O-[β-D- xylopyranosyl-( 1->3)-ß-D- glucuronopyranos yl]-28-O-[β-D- glucopyranosyl- (1->4)-α-L- rhamnopyranosyl- (1->2)-β-D- fucopyranosyl]- medicagenic acid) |
| 11 | 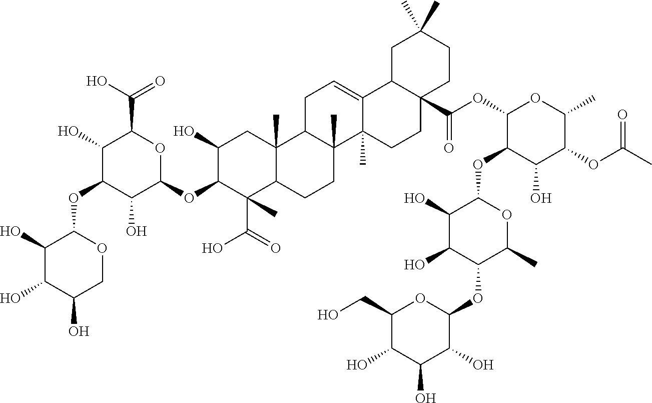 | Yossoside V (3-O-[β-D- xylopyranosyl- (1->3)-β-D- glucuronopyranos yl]-28-O-[β-D- glucopyranosyl- (1->4)-α-L- rhamnopyranosyl- (1->2)-4-acetyl-β- D-fucopyranosyl]- medicagenic acid) |
| 12 | 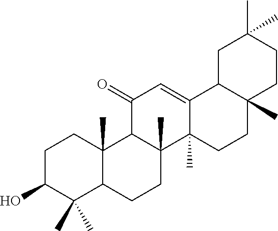 | 11-oxo-δ-Amyrin |
| 13 | 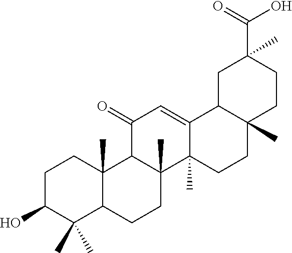 | glycyrrhetinic acid |
| 14 | 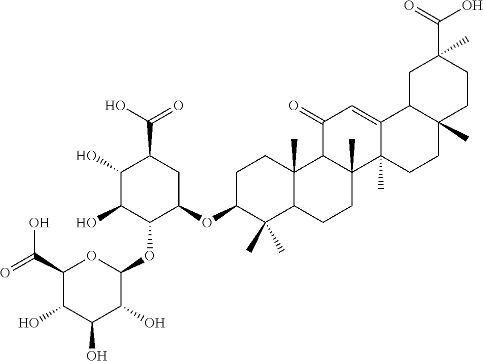 | glycyrrhizin |
| 15 | 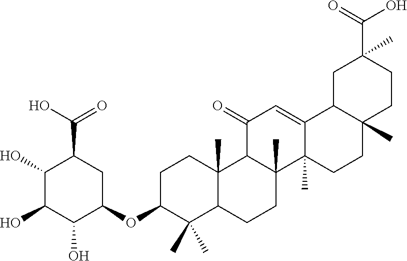 | glycyrrhetinic acid 3-0- monoglucuronide |
| 16 | not available | Yossoside Va |
| 17 | not available | Yossoside VI |
| 18 | not available | Yossoside VII |
| 19 | not available | Yossoside VIla |
| 20 | not available | Yossoside VIII |
| 21 | not available | Yossoside IX |
| 22 | not available | Yossoside X |
| 23 | not available | Yossoside XI |
| 24 | not available | Yossoside XII |
| 25 | 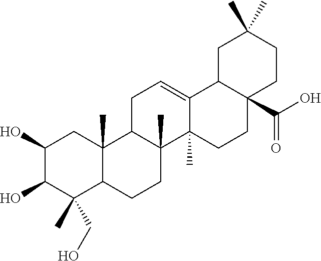 | Bayogenin |
| 26 | 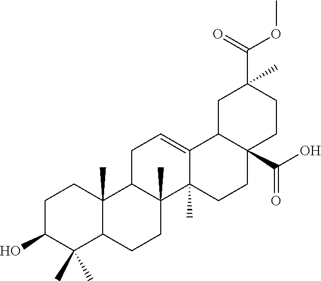 | Serjanic acid |
| 27 | 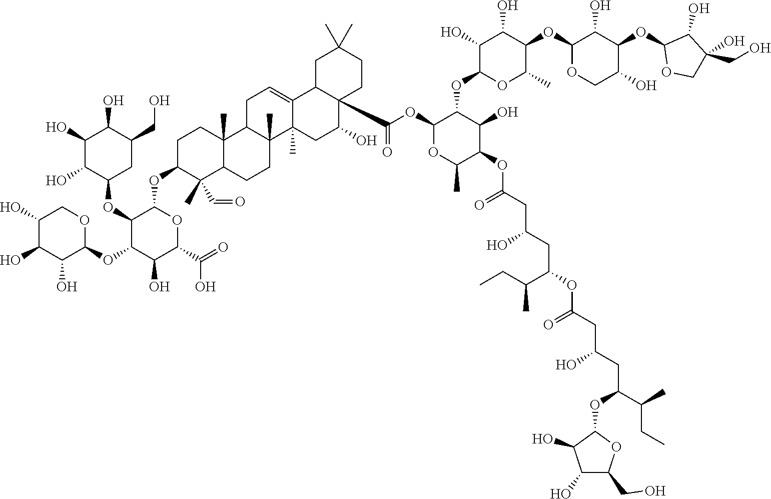 | QS-21 |
| 28 | 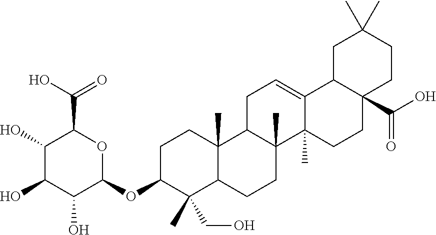 | Hederagenin- 3GIcA |
| 29 | 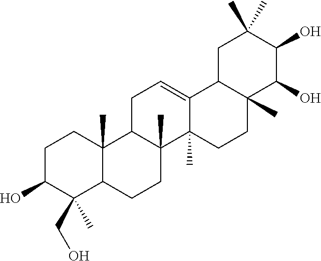 | Soyasapogenol A |
| 30 | 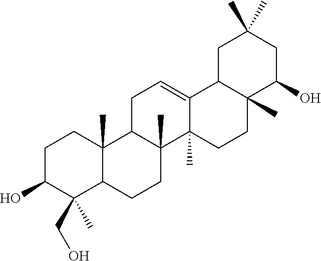 | Soyasapogenol B |
| 31 | 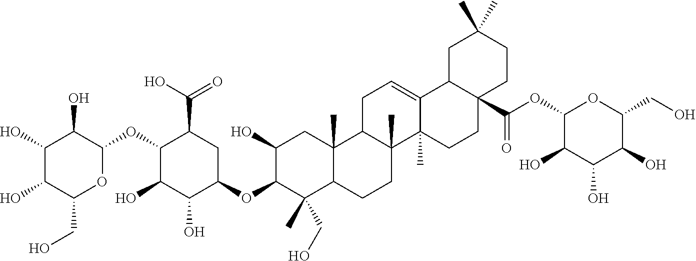 | Bayogenin-hexA- hex-hex |
| 32 | 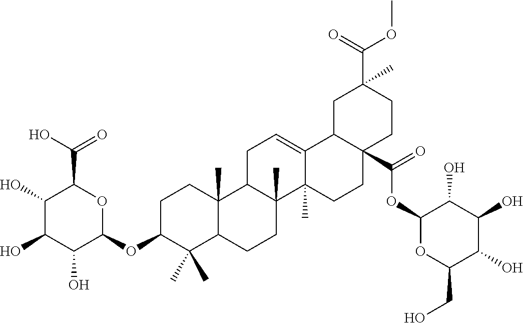 | Serjanic acid GlcA-glc |
| 33 | 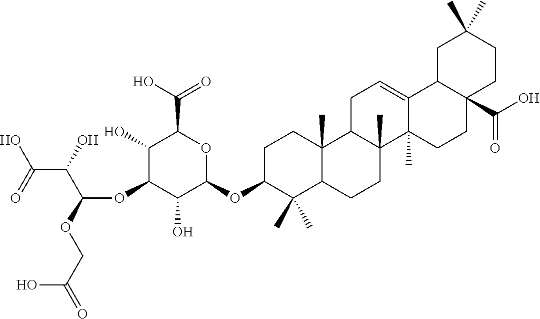 | betavulgaroside IV |
| 34 | 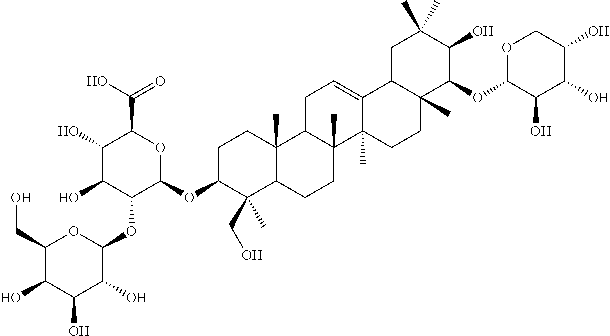 | Soyasapogenol A hexA-hex-pent |
| 35 | 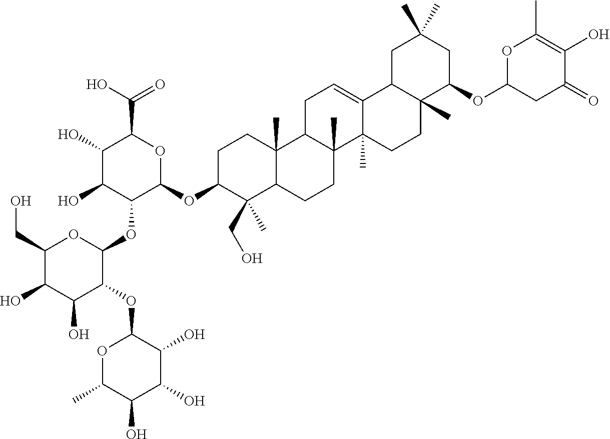 | soyasaponin VI (Saponin Bg) |
| 36 | 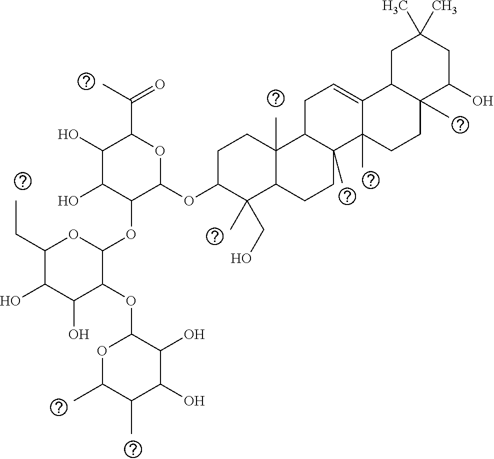 [SMILES: CCC1OC(OC2C(OC3CCC4(C)C(CCC5(C)C4CC=C4C6CC(C)(C)CC(O)C6(C)CCC45C)C3(C)CO)OC(C(C)=O)C(O)C2O)C(OC2OC(C)C(C)C(O)C2O)C(O)C1O] | Soyasaponin I (Saponin Bb) |

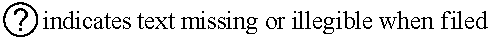

In some embodiments, derivatives of triterpenoid saponins comprise glycosylated derivatives of triterpenoid saponins.

In some embodiments, a triterpenoid saponins disclosed herein comprise any one of medicagenic acid 3-O-glucuronide (MA-3-GlcA) (Compound 6), Compound 7, Compound 8, Compound 9, Compound 10, Compound 11, glycyrrhizin (Compound 14), glycyrrhetinic acid 3-O-monoglucuronide (Compound 15), bayogenin (Compound 25), bayogenin-hexA-hex-hex (Compound 31), serjanic acid (Compound 26), serjanic acid-hexA-hex (Compound 32), soyasapogenol A (Compound 29), soyasapogenol B (Compound 30), soyasapogenol A-hexA-hex-pent (Compound 34), soyasaponin VI (Compound 35), soyasaponin (Compound 36), betavulgaroside IV (Compound 33), hederagenin-3GlcA, gypsogenin-3GlcA, gypsogenic acid-3GlcA, or a QS-21 adjuvant, or any combination thereof.

In some embodiments, a triterpenoid saponin comprises medicagenic acid 3-O-glucuronide (MA-3-GlcA) (Compound 6). In some embodiments, a triterpenoid saponin comprises Compound 7. In some embodiments, a triterpenoid saponin comprises Compound 8. In some embodiments, a triterpenoid saponin comprises Compound 9. In some embodiments, a triterpenoid saponin comprises Compound 10. In some embodiments, a triterpenoid saponin comprises Compound 11. In some embodiments, a triterpenoid saponin comprises glycyrrhizin (Compound 14). In some embodiments, a triterpenoid saponin comprises glycyrrhetinic acid 3-O-monoglucuronide (Compound 15). In some embodiments, a triterpenoid saponin comprises bayogenin (Compound 25). In some embodiments, a triterpenoid saponin comprises bayogenin-hexA-hex-hex (Compound 31). In some embodiments, a triterpenoid saponin comprises serjanic acid (Compound 26). In some embodiments, a triterpenoid saponin comprises serjanic acid-hexA-hex (Compound 32). In some embodiments, a triterpenoid saponin comprises soyasapogenol A (Compound 29). In some embodiments, a triterpenoid saponin comprises soyasapogenol B (Compound 30). In some embodiments, a triterpenoid saponin comprises soyasapogenol A-hexA-hex-pent (Compound 34). In some embodiments, a triterpenoid saponin comprises soyasaponin VI (Compound 35). In some embodiments, a triterpenoid saponin comprises soyasaponin I (Compound 36). In some embodiments, a triterpenoid saponin comprises betavulgaroside IV (Compound 33). In some embodiments, a triterpenoid saponin comprises hederagenin-3GlcA. In some embodiments, a triterpenoid saponin comprises gypsogenin-3GlcA. In some embodiments, atriterpenoid saponin comprises gypsogenic acid-3GlcA. In some embodiments, a triterpenoid saponin comprises a QS-21 adjuvant.

Altering the content of a triterpenoid saponin may in some embodiments, be commercially beneficial, for example but not limited to the ability to produce the sweetener, glycyrrhizin (Compound 14). In some embodiments, it is commercially beneficial to produce a triterpenoid saponin comprising any of a sweetener, foaming agent, emulsifier, preservative, anti-carcinogen, hypocholesterolemic agent, anti-inflammatory agent, anti-oxidant, biological adjuvant, anti-microbial agent, insecticidal agent, antifeedant, anti-fungal agent, or any combination thereof. In other embodiments, altering the content of a triterpenoid saponin may be beneficial, for example but not limited to reducing or eliminating bitter tasting triterpenoid saponins, for example but not limited to in quinoa, which is produced from a Chenopodium quinoa plant in the Caryophyllales order of plants. In other embodiments, altering the content of a triterpenoid saponin may be beneficial, for example but not limited to reducing or eliminating triterpenoid saponins that have hormone mimicking properties.

In some embodiments, a genetically modified cell disclosed herein comprises an increased content of at least one triterpenoid saponin selected from any one of medicagenic acid 3-O-glucuronide (MA-3-GlcA) (Compound 6), Compound 7, Compound 8, Compound 9, Compound 10, Compound 11, glycyrrhizin (Compound 14), Glycyrrhetinic acid 3-O-monoglucuronide (compound 15), bayogenin (Compound 25), bayogenin-hexA-hex-hex (Compound 31), serjanic acid (Compound 26), serjanic acid-hexA-hex (Compound 32), soyasapogenol A (Compound 29), soyasapogenol B (Compound 30), soyasapogenol A-hexA-hex-pent (Compound 34), soyasaponin VI (Compound 35), soyasaponin I (Compound 36), betavulgaroside IV (Compound 33), hederagenin-3GlcA, gypsogenin-3GlcA, gypsogenic acid-3GlcA, or a QS-21 adjuvant, or any combination thereof. In some embodiments, a genetically modified cell disclosed herein comprises an increased content of medicagenic acid 3-O-glucuronide (MA-3-GlcA) (Compound 6). In some embodiments, a genetically modified cell disclosed herein comprises an increased content of Compound 7. In some embodiments, a genetically modified cell disclosed herein comprises an increased content of Compound 8. In some embodiments, a genetically modified cell disclosed herein comprises an increased content of Compound 9. In some embodiments, a genetically modified cell disclosed herein comprises an increased content of Compound 10. In some embodiments, a genetically modified cell disclosed herein comprises an increased content of Compound 11. In some embodiments, a genetically modified cell disclosed herein comprises an increased content of glycyrrhizin (Compound 14). In some embodiments, a genetically modified cell disclosed herein comprises an increased content of Glycyrrhetinic acid 3-O-monoglucuronide (compound 15). In some embodiments, a genetically modified cell disclosed herein comprises an increased content of bayogenin (Compound 25). In some embodiments, a genetically modified cell disclosed herein comprises an increased content of bayogenin-hexA-hex-hex (Compound 31). In some embodiments, a genetically modified cell disclosed herein comprises an increased content of serjanic acid (Compound 26). In some embodiments, a genetically modified cell disclosed herein comprises an increased content of serjanic acid-hexA-hex (Compound 32). In some embodiments, a genetically modified cell disclosed herein comprises an increased content of soyasapogenol A (Compound 29). In some embodiments, a genetically modified cell disclosed herein comprises an increased content of soyasapogenol B (Compound 30). In some embodiments, a genetically modified cell disclosed herein comprises an increased content of soyasapogenol A-hexA-hex-pent (Compound 34). In some embodiments, a genetically modified cell disclosed herein comprises an increased content of soyasaponin VI (Compound 35). In some embodiments, a genetically modified cell disclosed herein comprises an increased content of soyasaponin I (Compound 36). In some embodiments, a genetically modified cell disclosed herein comprises an increased content of betavulgaroside IV (Compound 33). In some embodiments, a genetically modified cell disclosed herein comprises an increased content of hederagenin-3GlcA. In some embodiments, a genetically modified cell disclosed herein comprises an increased content of gypsogenin-3GlcA. In some embodiments, a genetically modified cell disclosed herein comprises an increased content of gypsogenic acid-3GlcA. In some embodiments, a genetically modified cell disclosed herein comprises an increased content of a QS-21 adjuvant.

In some embodiments, the genetically modified cell comprising said increased content comprises a plant cell, a yeast cell, an algal cell, an insect cell, or a bacterial. In some embodiments, the genetically modified cell comprising said increased content comprises a plant cell. In some embodiments, the genetically modified cell comprising said increased content comprises a yeast cell. In some embodiments, the genetically modified cell comprising said increased content comprises an algal cell. In some embodiments, the genetically modified cell comprising said increased content comprises an insect cell. In some embodiments, the genetically modified cell comprising said increased content comprises a bacterium.

In some embodiments, a genetically modified plant disclosed herein comprises an increased content of at least one triterpenoid saponin selected from any one of medicagenic acid 3-O-glucuronide (MA-3-GlcA) (Compound 6), Compound 7, Compound 8, Compound 9, Compound 10, Compound 11, glycyrrhizin (Compound 14), Glycyrrhetinic acid 3-O-monoglucuronide (compound 15), bayogenin (Compound 25), bayogenin-hexA-hex-hex (Compound 31), serjanic acid (Compound 26), serjanic acid-hexA-hex (Compound 32), soyasapogenol A (Compound 29), soyasapogenol B (Compound 30), soyasapogenol A-hexA-hex-pent (Compound 34), soyasaponin VI (Compound 35), soyasaponin I (Compound 36), betavulgaroside IV (Compound 33), hederagenin-3GlcA, gypsogenin-3GlcA, gypsogenic acid-3GlcA, or a QS-21 adjuvant, or any combination thereof. In some embodiments, a genetically modified plant disclosed herein comprises an increased content of medicagenic acid 3-O-glucuronide (MA-3-GlcA) (Compound 6). In some embodiments, a genetically modified plant disclosed herein comprises an increased content of Compound 7. In some embodiments, a genetically modified plant disclosed herein comprises an increased content of Compound 8. In some embodiments, a genetically modified plant disclosed herein comprises an increased content of Compound 9. In some embodiments, a genetically modified plant disclosed herein comprises an increased content of Compound 10. In some embodiments, a genetically modified plant disclosed herein comprises an increased content of Compound 11. In some embodiments, a genetically modified plant disclosed herein comprises an increased content of glycyrrhizin (Compound 14). In some embodiments, a genetically modified plant disclosed herein comprises an increased content of Glycyrrhetinic acid 3-O-monoglucuronide (compound 15). In some embodiments, a genetically modified plant disclosed herein comprises an increased content of bayogenin (Compound 25). In some embodiments, a genetically modified plant disclosed herein comprises an increased content of bayogenin-hexA-hex-hex (Compound 31). In some embodiments, a genetically modified plant disclosed herein comprises an increased content of serjanic acid (Compound 26). In some embodiments, a genetically modified plant disclosed herein comprises an increased content of serjanic acid-hexA-hex (Compound 32). In some embodiments, a genetically modified plant disclosed herein comprises an increased content of soyasapogenol A (Compound 29). In some embodiments, a genetically modified plant disclosed herein comprises an increased content of soyasapogenol B (Compound 30). In some embodiments, a genetically modified plant disclosed herein comprises an increased content of soyasapogenol A-hexA-hex-pent (Compound 34). In some embodiments, a genetically modified plant disclosed herein comprises an increased content of soyasaponin VI (Compound 35). In some embodiments, a genetically modified plant disclosed herein comprises an increased content of soyasaponin I (Compound 36). In some embodiments, a genetically modified plant disclosed herein comprises an increased content of betavulgaroside IV (Compound 33). In some embodiments, a genetically modified plant disclosed herein comprises an increased content of hederagenin-3GlcA. In some embodiments, a genetically modified plant disclosed herein comprises an increased content of gypsogenin-3GlcA. In some embodiments, a genetically modified plant disclosed herein comprises an increased content of gypsogenic acid-3GlcA. In some embodiments, a genetically modified plant disclosed herein comprises an increased content of a QS-21 adjuvant.

In some embodiments, a genetically modified plant disclosed herein comprises a decreased content of at least one triterpenoid saponin selected from any one of medicagenic acid 3-O-glucuronide (MA-3-GlcA) (Compound 6), Compound 7, Compound 8, Compound 9, Compound 10, Compound 11, glycyrrhizin (Compound 14), Glycyrrhetinic acid 3-O-monoglucuronide (compound 15), bayogenin (Compound 25), bayogenin-hexA-hex-hex (Compound 31), serjanic acid (Compound 26), serjanic acid-hexA-hex (Compound 32), soyasapogenol A (Compound 29), soyasapogenol B (Compound 30), soyasapogenol A-hexA-hex-pent (Compound 34), soyasaponin VI (Compound 35), soyasaponin I (Compound 36), betavulgaroside IV (Compound 33), hederagenin-3GlcA, gypsogenin-3GlcA, gypsogenic acid-3GlcA, or a QS-21 adjuvant, or any combination thereof. In some embodiments, a genetically modified plant disclosed herein comprises a decreased content of medicagenic acid 3-O-glucuronide (MA-3-GlcA) (Compound 6). In some embodiments, a genetically modified plant disclosed herein comprises a decreased content of Compound 7. In some embodiments, a genetically modified plant disclosed herein comprises a decreased content of Compound 8. In some embodiments, a genetically modified plant disclosed herein comprises a decreased content of Compound 9. In some embodiments, a genetically modified plant disclosed herein comprises a decreased content of Compound 10. In some embodiments, a genetically modified plant disclosed herein comprises a decreased content of Compound 11. In some embodiments, a genetically modified plant disclosed herein comprises a decreased content of glycyrrhizin (Compound 14). In some embodiments, a genetically modified plant disclosed herein comprises a decreased content of Glycyrrhetinic acid 3-O-monoglucuronide (compound 15). In some embodiments, a genetically modified plant disclosed herein comprises a decreased content of bayogenin (Compound 25). In some embodiments, a genetically modified plant disclosed herein comprises a decreased content of bayogenin-hexA-hex-hex (Compound 31). In some embodiments, a genetically modified plant disclosed herein comprises a decreased content of serjanic acid (Compound 26). In some embodiments, a genetically modified plant disclosed herein comprises a decreased content of serjanic acid-hexA-hex (Compound 32). In some embodiments, a genetically modified plant disclosed herein comprises a decreased content of soyasapogenol A (Compound 29). In some embodiments, a genetically modified plant disclosed herein comprises a decreased content of soyasapogenol B (Compound 30). In some embodiments, a genetically modified plant disclosed herein comprises a decreased content of soyasapogenol A-hexA-hex-pent (Compound 34). In some embodiments, a genetically modified plant disclosed herein comprises a decreased content of soyasaponin VI (Compound 35). In some embodiments, a genetically modified plant disclosed herein comprises a decreased content of soyasaponin I (Compound 36). In some embodiments, a genetically modified plant disclosed herein comprises a decreased content of betavulgaroside IV (Compound 33). In some embodiments, a genetically modified plant disclosed herein comprises a decreased content of hederagenin-3GlcA. In some embodiments, a genetically modified plant disclosed herein comprises a decreased content of gypsogenin-3GlcA. In some embodiments, a genetically modified plant disclosed herein comprises a decreased content of gypsogenic acid-3GlcA. In some embodiments, a genetically modified plant disclosed herein comprises a decreased content of a QS-21 adjuvant.

In some embodiments, a genetically modified plant disclosed herein comprises an increased content of at least one triterpenoid saponin and a decreased content of at least one triterpenoid saponin selected from any one of medicagenic acid 3-O-glucuronide (MA-3-GlcA) (Compound 6), Compound 7, Compound 8, Compound 9, Compound 10, Compound 11, glycyrrhizin (Compound 14), Glycyrrhetinic acid 3-O-monoglucuronide (compound 15), bayogenin (Compound 25), bayogenin-hexA-hex-hex (Compound 31), serjanic acid (Compound 26), serjanic acid-hexA-hex (Compound 32), soyasapogenol A (Compound 29), soyasapogenol B (Compound 30), soyasapogenol A-hexA-hex-pent (Compound 34), soyasaponin VI (Compound 35), soyasaponin I (Compound 36), betavulgaroside IV (Compound 33), hederagenin-3GlcA, gypsogenin-3GlcA, gypsogenic acid-3GlcA, or a QS-21 adjuvant, or any combination thereof.

In some embodiments, a method of producing a triterpenoid saponin in a genetically modified cell comprises producing at least one triterpenoid saponin selected from any one of medicagenic acid 3-O-glucuronide (MA-3-GlcA) (Compound 6), Compound 7, Compound 8, Compound 9, Compound 10, Compound 11, glycyrrhizin (Compound 14), Glycyrrhetinic acid 3-O-monoglucuronide (compound 15), bayogenin (Compound 25), bayogenin-hexA-hex-hex (Compound 31), serjanic acid (Compound 26), serjanic acid-hexA-hex (Compound 32), soyasapogenol A (Compound 29), soyasapogenol B (Compound 30), soyasapogenol A-hexA-hex-pent (Compound 34), soyasaponin VI (Compound 35), soyasaponin I (Compound 35), betavulgaroside IV (Compound 33), hederagenin-3GlcA, gypsogenin-3GlcA, gypsogenic acid-3GlcA, or a QS-21 adjuvant, or any combination thereof. In some embodiments, a method of producing a triterpenoid saponin in a genetically modified cell comprises producing medicagenic acid 3-O-glucuronide (MA-3-GlcA) (Compound 6). In some embodiments, a method of producing a triterpenoid saponin in a genetically modified cell comprises producing Compound 7. In some embodiments, a method of producing a triterpenoid saponin in a genetically modified cell comprises producing Compound 8. In some embodiments, a method of producing a triterpenoid saponin in a genetically modified cell comprises producing Compound 9. In some embodiments, a method of producing a triterpenoid saponin in a genetically modified cell comprises producing Compound 10. In some embodiments, a method of producing a triterpenoid saponin in a genetically modified cell comprises producing Compound 11. In some embodiments, a method of producing a triterpenoid saponin in a genetically modified cell comprises producing glycyrrhizin (Compound 14). In some embodiments, a method of producing a triterpenoid saponin in a genetically modified cell comprises producing Glycyrrhetinic acid 3-O-monoglucuronide (compound 15). In some embodiments, a method of producing a triterpenoid saponin in a genetically modified cell comprises producing bayogenin (Compound 25). In some embodiments, a method of producing a triterpenoid saponin in a genetically modified cell comprises producing bayogenin-hexA-hex-hex (Compound 31). In some embodiments, a method of producing a triterpenoid saponin in a genetically modified cell comprises producing serjanic acid (Compound 26). In some embodiments, a method of producing a triterpenoid saponin in a genetically modified cell comprises producing serjanic acid-hexA-hex (Compound 32). In some embodiments, a method of producing a triterpenoid saponin in a genetically modified cell comprises producing soyasapogenol A (Compound 29). In some embodiments, a method of producing a triterpenoid saponin in a genetically modified cell comprises producing soyasapogenol B (Compound 30). In some embodiments, a method of producing a triterpenoid saponin in a genetically modified cell comprises producing soyasapogenol A-hexA-hex-pent (Compound 34). In some embodiments, a method of producing a triterpenoid saponin in a genetically modified cell comprises producing soyasaponin VI (Compound 35). In some embodiments, a method of producing a triterpenoid saponin in a genetically modified cell comprises producing soyasaponin I (Compound 36). In some embodiments, a method of producing a triterpenoid saponin in a genetically modified cell comprises producing betavulgaroside IV (Compound 33). In some embodiments, a method of producing a triterpenoid saponin in a genetically modified cell comprises producing hederagenin-3GlcA. In some embodiments, a method of producing a triterpenoid saponin in a genetically modified cell comprises producing gypsogenin-3GlcA. In some embodiments, a method of producing a triterpenoid saponin in a genetically modified cell comprises producing gypsogenic acid-3GlcA. In some embodiments, a method of producing a triterpenoid saponin in a genetically modified cell comprises producing a QS-21 adjuvant.

In some embodiments, the method of producing at least one triterpenoid saponin in a genetically modified cell comprises producing at least one triterpenoid saponins in a plant cell, a yeast cell, an algal cell, an insect cell, or a bacterium. In some embodiments, the method of producing at least one triterpenoid saponin in a genetically modified cell comprises producing at least one triterpenoid saponins in a plant cell. In some embodiments, the method of producing at least one triterpenoid saponin in a genetically modified cell comprises producing at least one triterpenoid saponins in a yeast cell. In some embodiments, the method of producing at least one triterpenoid saponin in a genetically modified cell comprises producing at least one triterpenoid saponins in an algal cell. In some embodiments, the method of producing at least one triterpenoid saponin in a genetically modified cell comprises producing at least one triterpenoid saponins in an insect cell. In some embodiments, the method of producing at least one triterpenoid saponin in a genetically modified cell comprises producing at least one triterpenoid saponins in a bacterium. In some embodiments, the plant cell is comprised in a plant or plant part.

In some embodiments, a method of producing a triterpenoid saponin in a genetically modified plant comprises producing at least one triterpenoid saponin selected from any one of medicagenic acid 3-O-glucuronide (MA-3-GlcA) (Compound 6), Compound 7, Compound 8, Compound 9, Compound 10, Compound 11, glycyrrhizin (Compound 14), Glycyrrhetinic acid 3-O-monoglucuronide (compound 15), bayogenin (Compound 25), bayogenin-hexA-hex-hex (Compound 31), serjanic acid (Compound 26), serjanic acid-hexA-hex (Compound 32), soyasapogenol A (Compound 29), soyasapogenol B (Compound 30), soyasapogenol A-hexA-hex-pent (Compound 34), soyasaponin VI (Compound 35), soyasaponin I (Compound 36), betavulgaroside IV (Compound 33), hederagenin-3GlcA, gypsogenin-3GlcA, gypsogenic acid-3GlcA, or a QS-21 adjuvant, or any combination thereof. In some embodiments, a method of producing a triterpenoid saponin in a genetically modified plant comprises producing medicagenic acid 3-O-glucuronide (MA-3-GlcA) (Compound 6). In some embodiments, a method of producing a triterpenoid saponin in a genetically modified plant comprises producing Compound 7. In some embodiments, a method of producing a triterpenoid saponin in a genetically modified plant comprises producing Compound 8. In some embodiments, a method of producing a triterpenoid saponin in a genetically modified plant comprises producing Compound 9. In some embodiments, a method of producing a triterpenoid saponin in a genetically modified plant comprises producing Compound 10. In some embodiments, a method of producing a triterpenoid saponin in a genetically modified plant comprises producing Compound 11. In some embodiments, a method of producing a triterpenoid saponin in a genetically modified plant comprises producing glycyrrhizin (Compound 14). In some embodiments, a method of producing a triterpenoid saponin in a genetically modified plant comprises producing Glycyrrhetinic acid 3-O-monoglucuronide (compound 15). In some embodiments, a method of producing a triterpenoid saponin in a genetically modified plant comprises producing bayogenin (Compound 25). In some embodiments, a method of producing a triterpenoid saponin in a genetically modified plant comprises producing bayogenin-hexA-hex-hex (Compound 31). In some embodiments, a method of producing a triterpenoid saponin in a genetically modified plant comprises producing serjanic acid (Compound 26). In some embodiments, a method of producing a triterpenoid saponin in a genetically modified plant comprises producing serjanic acid-hexA-hex (Compound 32). In some embodiments, a method of producing a triterpenoid saponin in a genetically modified plant comprises producing soyasapogenol A (Compound 29). In some embodiments, a method of producing a triterpenoid saponin in a genetically modified plant comprises producing soyasapogenol B (Compound 30). In some embodiments, a method of producing a triterpenoid saponin in a genetically modified plant comprises producing soyasapogenol A-hexA-hex-pent (Compound 34). In some embodiments, a method of producing a triterpenoid saponin in a genetically modified plant comprises producing soyasaponin VI (Compound 35). In some embodiments, a method of producing a triterpenoid saponin in a genetically modified plant comprises producing soyasaponin I (Compound 36). In some embodiments, a method of producing a triterpenoid saponin in a genetically modified plant comprises producing betavulgaroside IV (Compound 33). In some embodiments, a method of producing a triterpenoid saponin in a genetically modified plant comprises producing hederagenin-3GlcA. In some embodiments, a method of producing a triterpenoid saponin in a genetically modified plant comprises producing gypsogenin-3GlcA. In some embodiments, a method of producing a triterpenoid saponin in a genetically modified plant comprises producing gypsogenic acid-3GlcA. In some embodiments, a method of producing a triterpenoid saponin in a genetically modified plant comprises producing a QS-21 adjuvant.

In some embodiments, a method of producing a triterpenoid saponin in an in vitro translation system comprises producing at least one triterpenoid saponin selected from any one of medicagenic acid 3-O-glucuronide (MA-3-GlcA) (Compound 6), Compound 7, Compound 8, Compound 9, Compound 10, Compound 11, glycyrrhizin (Compound 14), Glycyrrhetinic acid 3-O-monoglucuronide (compound 15), bayogenin (Compound 25), bayogenin-hexA-hex-hex (Compound 31), serjanic acid (Compound 26), serjanic acid-hexA-hex (Compound 32), soyasapogenol A (Compound 29), soyasapogenol B (Compound 30), soyasapogenol A-hexA-hex-pent (Compound 34), soyasaponin VI (Compound 35), soyasapnin I (Compound 36), betavulgaroside IV (Compound 33), hederagenin-3GlcA, gypsogenin-3GlcA, gypsogenic acid-3GlcA, or a QS-21 adjuvant, or any combination thereof. In some embodiments, a method of producing a triterpenoid saponin in an in vitro translation system comprises producing medicagenic acid 3-O-glucuronide (MA-3-GlcA) (Compound 6). In some embodiments, a method of producing a triterpenoid saponin in an in vitro translation system comprises producing Compound 7. In some embodiments, a method of producing a triterpenoid saponin in an in vitro translation system comprises producing Compound 8. In some embodiments, a method of producing a triterpenoid saponin in an in vitro translation system comprises producing Compound 9. In some embodiments, a method of producing a triterpenoid saponin in an in vitro translation system comprises producing Compound 10. In some embodiments, a method of producing a triterpenoid saponin in an in vitro translation system comprises producing Compound 11. In some embodiments, a method of producing a triterpenoid saponin in an in vitro translation system comprises producing glycyrrhizin (Compound 14). In some embodiments, a method of producing a triterpenoid saponin in an in vitro translation system comprises producing Glycyrrhetinic acid 3-O-monoglucuronide (compound 15). In some embodiments, a method of producing a triterpenoid saponin in an in vitro translation system comprises producing bayogenin (Compound 25). In some embodiments, a method of producing a triterpenoid saponin in an in vitro translation system comprises producing bayogenin-hexA-hex-hex (Compound 31). In some embodiments, a method of producing a triterpenoid saponin in an in vitro translation system comprises producing serjanic acid (Compound 26). In some embodiments, a method of producing a triterpenoid saponin in an in vitro translation system comprises producing serjanic acid-hexA-hex (Compound 32). In some embodiments, a method of producing a triterpenoid saponin in an in vitro translation system comprises producing soyasapogenol A (Compound 29). In some embodiments, a method of producing a triterpenoid saponin in an in vitro translation system comprises producing soyasapogenol B (Compound 30). In some embodiments, a method of producing a triterpenoid saponin in an in vitro translation system comprises producing soyasapogenol A-hexA-hex-pent (Compound 34). In some embodiments, a method of producing a triterpenoid saponin in an in vitro translation system comprises producing soyasaponin VI (Compound 35). In some embodiments, a method of producing a triterpenoid saponin in an in vitro translation system comprises producing soyasaponin I (Compound 36). In some embodiments, a method of producing a triterpenoid saponin in an in vitro translation system comprises producing betavulgaroside IV (Compound 33). In some embodiments, a method of producing a triterpenoid saponin in an in vitro translation system comprises producing hederagenin-3GlcA. In some embodiments, a method of producing a triterpenoid saponin in an in vitro translation system comprises producing gypsogenin-3GlcA. In some embodiments, a method of producing a triterpenoid saponin in an in vitro translation system comprises producing gypsogenic acid-3GlcA. In some embodiments, a method of producing a triterpenoid saponin in an in vitro translation system comprises producing a QS-21 adjuvant.

In some embodiments, a method of reducing at least one triterpenoid saponin comprises reducing at least one triterpenoid saponin selected from any one of medicagenic acid 3-O-glucuronide (MA-3-GlcA) (Compound 6), Compound 7, Compound 8, Compound 9, Compound 10, Compound 11, glycyrrhizin (Compound 14), Glycyrrhetinic acid 3-O-monoglucuronide (compound 15), bayogenin (Compound 25), bayogenin-hexA-hex-hex (Compound 31), serjanic acid (Compound 26), serjanic acid-hexA-hex (Compound 32), soyasapogenol A (Compound 29), soyasapogenol B (Compound 30), soyasapogenol A-hexA-hex-pent (Compound 34), soyasaponin VI (Compound 35), soyasaponin I (Compound 36), betavulgaroside IV (Compound 33), hederagenin-3GlcA, gypsogenin-3GlcA, gypsogenic acid-3GlcA, or a QS-21 adjuvant, or any combination thereof. In some embodiments, a method of reducing the content of at least one triterpenoid saponin comprising medicagenic acid 3-O-glucuronide (MA-3-GlcA) (Compound 6). In some embodiments, a method of reducing at least one triterpenoid saponin comprises reducing Compound 7. In some embodiments, a method of reducing at least one triterpenoid saponin comprises reducing Compound 8. In some embodiments, a method of reducing at least one triterpenoid saponin comprises reducing Compound 9. In some embodiments, a method of reducing at least one triterpenoid saponin comprises reducing Compound 10. In some embodiments, a method of reducing at least one triterpenoid saponin comprises reducing Compound 11. In some embodiments, a method of reducing at least one triterpenoid saponin comprises reducing glycyrrhizin (Compound 14). In some embodiments, a method of reducing at least one triterpenoid saponin comprises reducing Glycyrrhetinic acid 3-O-monoglucuronide (compound 15). In some embodiments, a method of reducing at least one triterpenoid saponin comprises reducing bayogenin (Compound 25). In some embodiments, a method of reducing at least one triterpenoid saponin comprises reducing bayogenin-hexA-hex-hex (Compound 31). In some embodiments, a method of reducing at least one triterpenoid saponin comprises reducing serjanic acid (Compound 26). In some embodiments, a method of reducing at least one triterpenoid saponin comprises reducing serjanic acid-hexA-hex (Compound 32). In some embodiments, a method of reducing at least one triterpenoid saponin comprises reducing soyasapogenol A (Compound 29). In some embodiments, a method of reducing at least one triterpenoid saponin comprises reducing soyasapogenol B (Compound 30). In some embodiments, a method of reducing at least one triterpenoid saponin comprises reducing soyasapogenol A-hexA-hex-pent (Compound 34). In some embodiments, a method of reducing at least one triterpenoid saponin comprises reducing soyasaponin VI (Compound 35). In some embodiments, a method of reducing at least one triterpenoid saponin comprises reducing soyasaponin I (Compound 36). In some embodiments, a method of reducing at least one triterpenoid saponin comprises reducing betavulgaroside IV (Compound 33). In some embodiments, a method of reducing at least one triterpenoid saponin comprises reducing hederagenin-3GlcA. In some embodiments, a method of reducing at least one triterpenoid saponin comprises reducing gypsogenin-3GlcA. In some embodiments, a method of reducing at least one triterpenoid saponin comprises reducing gypsogenic acid-3GlcA. In some embodiments, a method of reducing at least one triterpenoid saponin comprises reducing a QS-21 adjuvant.

In some embodiments, a method of increasing at least one triterpenoid saponin comprises increasing at least one triterpenoid saponin selected from any one of medicagenic acid 3-O-glucuronide (MA-3-GlcA) (Compound 6), Compound 7, Compound 8, Compound 9, Compound 10, Compound 11, glycyrrhizin (Compound 14), Glycyrrhetinic acid 3-O-monoglucuronide (compound 15), bayogenin (Compound 25), bayogenin-hexA-hex-hex (Compound 31), serjanic acid (Compound 26), serjanic acid-hexA-hex (Compound 32), soyasapogenol A (Compound 29), soyasapogenol B (Compound 30), soyasapogenol A-hexA-hex-pent (Compound 34), soyasaponin VI (Compound 35), soyasaponin I (Compound 36) betavulgaroside IV (Compound 33), hederagenin-3GlcA, gypsogenin-3GlcA, gypsogenic acid-3GlcA, or a QS-21 adjuvant, or any combination thereof. In some embodiments, a method of increasing the content of at least one triterpenoid saponin comprising medicagenic acid 3-O-glucuronide (MA-3-GlcA) (Compound 6). In some embodiments, a method of increasing at least one triterpenoid saponin comprises increasing Compound 7. In some embodiments, a method of increasing at least one triterpenoid saponin comprises increasing Compound 8. In some embodiments, a method of increasing at least one triterpenoid saponin comprises increasing Compound 9. In some embodiments, a method of increasing at least one triterpenoid saponin comprises increasing Compound 10. In some embodiments, a method of increasing at least one triterpenoid saponin comprises increasing Compound 11. In some embodiments, a method of increasing at least one triterpenoid saponin comprises increasing glycyrrhizin (Compound 14). In some embodiments, a method of increasing at least one triterpenoid saponin comprises increasing Glycyrrhetinic acid 3-O-monoglucuronide (compound 15). In some embodiments, a method of increasing at least one triterpenoid saponin comprises increasing bayogenin (Compound 25). In some embodiments, a method of increasing at least one triterpenoid saponin comprises increasing bayogenin-hexA-hex-hex (Compound 31). In some embodiments, a method of increasing at least one triterpenoid saponin comprises increasing serjanic acid (Compound 26). In some embodiments, a method of increasing at least one triterpenoid saponin comprises increasing serjanic acid-hexA-hex (Compound 32). In some embodiments, a method of increasing at least one triterpenoid saponin comprises increasing soyasapogenol A (Compound 29). In some embodiments, a method of increasing at least one triterpenoid saponin comprises increasing soyasapogenol B (Compound 30). In some embodiments, a method of increasing at least one triterpenoid saponin comprises increasing soyasapogenol A-hexA-hex-pent (Compound 34). In some embodiments, a method of increasing at least one triterpenoid saponin comprises increasing soyasaponin VI (Compound 35). In some embodiments, a method of increasing at least one triterpenoid saponin comprises increasing soyasaponin I (Compound 36). In some embodiments, a method of increasing at least one triterpenoid saponin comprises increasing betavulgaroside IV (Compound 33). In some embodiments, a method of increasing at least one triterpenoid saponin comprises increasing hederagenin-3GlcA. In some embodiments, a method of increasing at least one triterpenoid saponin comprises increasing gypsogenin-3GlcA. In some embodiments, a method of increasing at least one triterpenoid saponin comprises increasing gypsogenic acid-3GlcA. In some embodiments, a method of increasing at least one triterpenoid saponin comprises increasing a QS-21 adjuvant.

In some embodiments, the content of both at least one triterpenoid saponin and at least one triterpenoid saponin biosynthetic intermediate are altered. In some embodiments, an at least one triterpenoid saponin and an at least one triterpenoid saponin biosynthetic intermediate are increased. In some embodiments, an at least one triterpenoid saponins and an at least one triterpenoid saponin biosynthetic intermediate are decreased. In some embodiments, an at least one triterpenoid saponin is increased and an at least one triterpenoid saponin biosynthetic intermediate is decreased. In some embodiments, an at least one triterpenoid saponin is decreased and an at least one triterpenoid saponin biosynthetic intermediate is increased. In some embodiments, the content of a triterpenoid saponin is altered without measurably altering the content of a triterpenoid saponin intermediate. In some embodiments, the content of a triterpenoid saponin intermediate is altered without measurably altering the content of a triterpenoid saponin.

The term “intermediate” may be used interchangeably in some embodiments with the term “biosynthetic intermediate”, having all the same qualities and meanings.

In some embodiments, a biosynthetic intermediate of a triterpenoid saponin comprises Compound 1, Compound 2, medicagenic acid (Compound 5), oleanolic acid (Compound 3), oleanolic acid-3GlcA, augustic acid (Compound 4), augustic acid-3GlcA, or glycyrrhetinic acid (Compound 13), or any combination thereof.

In some embodiments, a genetically modified cell disclosed herein comprises an increased content of at least one biosynthetic intermediate of a triterpenoid saponin comprising Compound 1, Compound 2, medicagenic acid (Compound 5), oleanolic acid (Compound 3), oleanolic acid-3GlcA, augustic acid (Compound 4), augustic acid-3GlcA, or glycyrrhetinic acid (Compound 13), or any combination thereof. In some embodiments, a genetically modified cell comprises an increased content of at least one biosynthetic intermediate of a triterpenoid saponin comprising Compound 1. In some embodiments, a genetically modified cell comprises an increased content of at least one biosynthetic intermediate of a triterpenoid saponin comprising Compound 2. In some embodiments, a genetically modified cell comprises an increased content of at least one biosynthetic intermediate of a triterpenoid saponin comprising medicagenic acid (Compound 5). In some embodiments, a genetically modified cell comprises an increased content of at least one biosynthetic intermediate of a triterpenoid saponin comprising oleanolic acid (Compound 3). In some embodiments, a genetically modified cell comprises an increased content of at least one biosynthetic intermediate of a triterpenoid saponin comprising oleanolic acid-3GlcA. In some embodiments, a genetically modified cell comprises an increased content of at least one biosynthetic intermediate of a triterpenoid saponin comprising augustic acid (Compound 4). In some embodiments, a genetically modified cell comprises an increased content of at least one biosynthetic intermediate of a triterpenoid saponin comprising augustic acid-3GlcA. In some embodiments, a genetically modified cell comprises an increased content of at least one biosynthetic intermediate of a triterpenoid saponin comprising glycyrrhetinic acid (Compound 13).

In some embodiments, a genetically modified plant disclosed herein comprises a decreased content of at least one biosynthetic intermediate of a triterpenoid saponin comprising Compound 1, Compound 2, medicagenic acid (Compound 5), oleanolic acid (Compound 3), oleanolic acid-3GlcA, augustic acid (Compound 4), augustic acid-3GlcA, or glycyrrhetinic acid (Compound 13), or any combination thereof. In some embodiments, a genetically modified plant comprises a decreased content of at least one biosynthetic intermediate of a triterpenoid saponin comprising Compound 1. In some embodiments, a genetically modified plant comprises a decreased content of at least one biosynthetic intermediate of a triterpenoid saponin comprising Compound 2. In some embodiments, a genetically modified plant comprises a decreased content of at least one biosynthetic intermediate of a triterpenoid saponin comprising medicagenic acid (Compound 5). In some embodiments, a genetically modified plant comprises a decreased content of at least one biosynthetic intermediate of a triterpenoid saponin comprising oleanolic acid (Compound 3). In some embodiments, a genetically modified plant comprises a decreased content of at least one biosynthetic intermediate of a triterpenoid saponin comprising oleanolic acid-3GlcA. In some embodiments, a genetically modified plant comprises a decreased content of at least one biosynthetic intermediate of a triterpenoid saponin comprising augustic acid (Compound 4). In some embodiments, a genetically modified plant comprises a decreased content of at least one biosynthetic intermediate of a triterpenoid saponin comprising augustic acid-3GlcA. In some embodiments, a genetically modified plant comprises a decreased content of at least one biosynthetic intermediate of a triterpenoid saponin comprising glycyrrhetinic acid (Compound 13).

In some embodiments, a genetically modified plant disclosed herein comprises an increased content of at least one triterpenoid saponin biosynthetic intermediate and a decreased content of at least one triterpenoid saponin biosynthetic intermediate, said intermediate selected from any one of Compound 1, Compound 2, medicagenic acid (Compound 5), oleanolic acid (Compound 3), oleanolic acid-3GlcA, augustic acid (Compound 4), augustic acid-3GlcA, or glycyrrhetinic acid (Compound 13), or any combination thereof.

In some embodiments, a method of reducing at least one triterpenoid saponin comprises reducing at least one triterpenoid saponin biosynthetic intermediate selected from any one of Compound 1, Compound 2, medicagenic acid (Compound 5), oleanolic acid (Compound 3), oleanolic acid-3GlcA, augustic acid (Compound 4), augustic acid-3GlcA, or glycyrrhetinic acid (Compound 13), or any combination thereof. In some embodiments, a method of reducing at least one triterpenoid saponin comprises reducing at least Compound 1. In some embodiments, a method of reducing at least one triterpenoid saponin comprises reducing at least Compound 2. In some embodiments, a method of reducing at least one triterpenoid saponin comprises reducing at least medicagenic acid (Compound 5) In some embodiments, a method of reducing at least one triterpenoid saponin comprises reducing at least oleanolic acid (Compound 3) In some embodiments, a method of reducing at least one triterpenoid saponin comprises reducing at least oleanolic acid-3GlcA In some embodiments, a method of reducing at least one triterpenoid saponin comprises reducing at least augustic acid (Compound 4) In some embodiments, a method of reducing at least one triterpenoid saponin comprises reducing at least augustic acid-3GlcA In some embodiments, a method of reducing at least one triterpenoid saponin comprises reducing at least glycyrrhetinic acid (Compound 13).

In some embodiments, a method of increasing at least one triterpenoid saponin comprises increasing at least one triterpenoid saponin biosynthetic intermediate selected from any one of Compound 1, Compound 2, medicagenic acid (Compound 5), oleanolic acid (Compound 3), oleanolic acid-3GlcA, augustic acid (Compound 4), augustic acid-3GlcA, or glycyrrhetinic acid (Compound 13), or any combination thereof. In some embodiments, a method of increasing at least one triterpenoid saponin comprises increasing at least Compound 1. In some embodiments, a method of increasing at least one triterpenoid saponin comprises increasing at least Compound 2. In some embodiments, a method of increasing at least one triterpenoid saponin comprises increasing at least medicagenic acid (Compound 5) In some embodiments, a method of increasing at least one triterpenoid saponin comprises increasing at least oleanolic acid (Compound 3) In some embodiments, a method of increasing at least one triterpenoid saponin comprises increasing at least oleanolic acid-3GlcA In some embodiments, a method of increasing at least one triterpenoid saponin comprises increasing at least augustic acid (Compound 4) In some embodiments, a method of increasing at least one triterpenoid saponin comprises increasing at least augustic acid-3GlcA In some embodiments, a method of increasing at least one triterpenoid saponin comprises increasing at least glycyrrhetinic acid (Compound 13).

In some embodiments, triterpenoid saponins biosynthetic intermediate comprises any Compound 1, Compound 2, medicagenic acid (Compound 5), oleanolic acid (Compound 3), oleanolic acid-3GlcA, augustic acid (Compound 4), augustic acid-3GlcA, or glycyrrhetinic acid (Compound 13), or a combination thereof.

Unexpectedly, the present disclosure now shows that levels of triterpenoid saponins or their derivatives, metabolites, or biosynthetic intermediates can be increased in cells, for example plant cells or yeast cells or algal cells, or insect cells, or bacterium, or plants by genetically modifying the cell or plant to express at least one heterologous gene encoding an enzyme, for example an enzyme or enzymes of the triterpenoid saponin biosynthetic pathway. In other embodiments, described and exemplified herein are methods of genetically modifying an at least one endogenous gene in a plant cell, for example but not limited to a CSLG gene, to regulate expression, activity, or stability, or any combination thereof. The Examples below disclose enzyme activities and enzymes previously unknown to be part of the triterpenoid saponin biosynthetic pathway. Without this knowledge, production of the triterpenoid saponin compounds was not possible.

In some embodiments, the triterpenoid saponin metabolic pathway can result in cells or plants comprising elevated content of triterpenoid saponins, a derivative thereof, a metabolite thereof, or a biosynthetic intermediate thereof, or in plants having an increased content of these compounds in the plant or plant parts. In some embodiments, plants including but not limited to crop plants are produced, wherein the crop has an increased content of a useful triterpenoid saponin or triterpenoid saponins. In some embodiments, disclosed herein are the means and methods for producing cells including but not limited to plant cells, yeast, or algal cells; plants including but not limited to crop plants, or a part of a plant; having increased levels of a triterpenoid saponin, or triterpenoid saponins, or derivatives thereof, or metabolites thereof, or a biosynthetic intermediate thereof. Alternatively, or additionally, controlling the expression of genes disclosed herein may be used for the production of desired triterpenoid saponins for further use, for example in the pharmaceutical industry or for the formulation of dietary or other supplements, for example but not limited to sweeteners. In some embodiments, these high value saponins may be purified and used, e.g., as sweeteners, foaming agents, emulsifiers, preservatives, anti-carcinogens, hypocholesterolemic agents, anti-inflammatory agents, anti-oxidants, biological adjuvants, anti-microbial agents, insecticidal agents, anti-feedants, or anti-fungal agents, or any combination thereof. The cells and plants disclosed herein comprise compounds of significant nutritional, pharmaceutical, and commercial value.

In some embodiments, a genetically modified cell or genetically modified plant or plant part comprising at least one genetically modified plant cell, as described herein, comprises an altered content of at least a triterpenoid saponin, a derivative thereof, a metabolite thereof, or a biosynthetic intermediate thereof, compared to a corresponding unmodified cell or unmodified plant. In some embodiments, an altered content comprises an increased content. In some embodiments, for example, the genetically modified cell or genetically modified plant has an increased content of at least a triterpenoid saponin, a derivative thereof, a metabolite thereof, or a biosynthetic intermediate thereof, or any combination thereof.

In some embodiments, the genetically modified cell or genetically modified plant or plant part comprising at least one genetically modified plant cell, has an increased content of at least one triterpenoid saponin. In some embodiments, the genetically modified cell or genetically modified plant or plant part comprising at least one genetically modified plant cell, has an increased content of at least two triterpenoid saponins. In some embodiments, the genetically modified cell or genetically modified plant or plant part comprising at least one genetically modified plant cell, has an increased content of at least three triterpenoid saponins. In some embodiments, the genetically modified cell or genetically modified plant or plant part comprising at least one genetically modified plant cell, has an increased content of at least four triterpenoid saponins. In some embodiments, the genetically modified cell or genetically modified plant or plant part comprising at least one genetically modified plant cell, has an increased content of at least five triterpenoid saponins. In some embodiments, the genetically modified cell or genetically modified plant or plant part comprising at least one genetically modified plant cell, has an increased content of at least six triterpenoid saponins.

In some embodiments, the genetically modified cell or genetically modified plant or plant part comprising at least one genetically modified plant cell, has an increased content of at least a derivative of a triterpenoid saponin. In some embodiments, the genetically modified cell or genetically modified plant or plant part comprising at least one genetically modified plant cell, has an increased content of at least two derivatives of a triterpenoid saponin or of triterpenoid saponins. In some embodiments, the genetically modified cell or genetically modified plant or plant part comprising at least one genetically modified plant cell, has an increased content of at least three derivatives of a triterpenoid saponin or of triterpenoid saponins. In some embodiments, the genetically modified cell or genetically modified plant or plant part comprising at least one genetically modified plant cell, has an increased content of at least four derivatives of a triterpenoid saponin or of triterpenoid saponins. In some embodiments, the genetically modified cell or genetically modified plant or plant part comprising at least one genetically modified plant cell, has an increased content of at least five derivatives of a triterpenoid saponin or of triterpenoid saponins. In some embodiments, the genetically modified cell or genetically modified plant or plant part comprising at least one genetically modified plant cell, has an increased content of at least six derivatives of a triterpenoid saponin or of triterpenoid saponins.

In some embodiments, the genetically modified cell or genetically modified plant or plant part comprising at least one genetically modified plant cell, has an increased content of at least a metabolite of a triterpenoid saponin. In some embodiments, the genetically modified cell or genetically modified plant or plant part comprising at least one genetically modified plant cell, has an increased content of at least two metabolites of a triterpenoid saponin or of triterpenoid saponins. In some embodiments, the genetically modified cell or genetically modified plant or plant part comprising at least one genetically modified plant cell, has an increased content of at least three metabolites of a triterpenoid saponin or of triterpenoid saponins. In some embodiments, the genetically modified cell or genetically modified plant or plant part comprising at least one genetically modified plant cell, has an increased content of at least four metabolites of a triterpenoid saponin or of triterpenoid saponins. In some embodiments, the genetically modified cell or genetically modified plant or plant part comprising at least one genetically modified plant cell, has an increased content of at least five metabolites of a triterpenoid saponin or of triterpenoid saponins. In some embodiments, the genetically modified cell or genetically modified plant or plant part comprising at least one genetically modified plant cell, has an increased content of at least six metabolites of a triterpenoid saponin or of triterpenoid saponins.

In some embodiments, the genetically modified cell or genetically modified plant or plant part comprising at least one genetically modified plant cell, has an increased content of at least a biosynthetic intermediate of a triterpenoid saponin. In some embodiments, the genetically modified cell or genetically modified plant or plant part comprising at least one genetically modified plant cell, has an increased content of at least two biosynthetic intermediates of a triterpenoid saponin or of triterpenoid saponins. In some embodiments, the genetically modified cell or genetically modified plant or plant part comprising at least one genetically modified plant cell, has an increased content of at least three biosynthetic intermediates of a triterpenoid saponin or of triterpenoid saponins. In some embodiments, the genetically modified cell or genetically modified plant or plant part comprising at least one genetically modified plant cell, has an increased content of at least four biosynthetic intermediates of a triterpenoid saponin or of triterpenoid saponins. In some embodiments, the genetically modified cell or genetically modified plant or plant part comprising at least one genetically modified plant cell, has an increased content of at least five biosynthetic intermediates of a triterpenoid saponin or of triterpenoid saponins. In some embodiments, the genetically modified cell or genetically modified plant or plant part comprising at least one genetically modified plant cell, has an increased content of at least six biosynthetic intermediates of a triterpenoid saponin or of triterpenoid saponins.

As skilled artisan would recognize that the terms “content” and “level” in reference to a triterpenoid saponin, a derivative thereof, a metabolite thereof, or a biosynthetic intermediate thereof, encompasses the quantity of the compound, for example the quantity of a triterpenoid saponin, a derivative thereof, a metabolite thereof, or a biosynthetic intermediate thereof in a genetically modified cell or in a genetically modified plant, compared with a control cell or control plant. In this context, the terms “content” and “level” may be used interchangeably having all the same meanings and qualities.

In some embodiments, a triterpenoid saponin having increased content comprises a sweetener, foaming agent, emulsifier, preservative, anti-carcinogen, hypocholesterolemic agent, anti-inflammatory agent, anti-oxidant, biological adjuvant, anti-microbial agent, insecticidal agent, antifeedant, anti-fungal agent, or any combination thereof. In some embodiments, a triterpenoid saponin having increased content comprises triterpenoid saponin selected from a medicagenic acid 3-O-glucuronide (MA-3-GlcA) (Compound 6), Compound 7, Compound 8, Compound 9, Compound 10, Compound 11, glycyrrhizin (Compound 14), Glycyrrhetinic acid 3-O-monoglucuronide (compound 15), bayogenin (Compound 25), bayogenin-hexA-hex-hex (Compound 31), serjanic acid (Compound 26), serjanic acid-hexA-hex (Compound 32), soyasapogenol A (Compound 29), soyasapogenol B (Compound 30), soyasapogenol A-hexA-hex-pent (Compound 34), soyasaponin VI (Compound 35), soyasaponin I (Compound 36), betavulgaroside IV (Compound 33), hederagenin-3GlcA, gypsogenin-3GlcA, gypsogenic acid-3GlcA, or a QS-21 adjuvant, or any combination thereof. In some embodiments, a triterpenoid saponin having increased content comprises at least 1, 2, 3, 4, 5, 6, or more triterpenoid saponin selected from medicagenic acid 3-O-glucuronide (MA-3-GlcA) (Compound 6), Compound 7, Compound 8, Compound 9, Compound 10, Compound 11, glycyrrhizin (Compound 14), Glycyrrhetinic acid 3-O-monoglucuronide (compound 15), bayogenin (Compound 25), bayogenin-hexA-hex-hex (Compound 31), serjanic acid (Compound 26), serjanic acid-hexA-hex (Compound 32), soyasapogenol A (Compound 29), soyasapogenol B (Compound 30), soyasapogenol A-hexA-hex-pent (Compound 34), soyasaponin VI (Compound 35), soyasaponin I (Compound 36), betavulgaroside IV (Compound 33), hederagenin-3GlcA, gypsogenin-3GlcA, gypsogenic acid-3GlcA, or a QS-21 adjuvant, or any combination thereof.

In some embodiments, a triterpenoid saponin having increased content comprises at least 1, 2, 3, 4, 5, 6, or more triterpenoid saponin selected from medicagenic acid 3-O-glucuronide (MA-3-GlcA) (Compound 6), Compound 7, Compound 8, Compound 9, Compound 10, Compound 11, glycyrrhizin (Compound 14), Glycyrrhetinic acid 3-O-monoglucuronide (compound 15), bayogenin (Compound 25), bayogenin-hexA-hex-hex (Compound 31), serjanic acid (Compound 26), serjanic acid-hexA-hex (Compound 32), soyasapogenol A (Compound 29), soyasapogenol B (Compound 30), soyasapogenol A-hexA-hex-pent (Compound 34), soyasaponin VI (Compound 35), soyasaponin I (Compound 36), betavulgaroside IV (Compound 33), hederagenin-3GlcA, gypsogenin-3GlcA, gypsogenic acid-3GlcA, or a QS-21 adjuvant, or any combination thereof.

In some embodiments, a triterpenoid saponin having increased content in a genetically modified cell, for example a plant cell or a yeast or an algal cell or an insect cell or a bacterium, or in a genetically modified plant or part thereof, comprises triterpenoid saponin selected from medicagenic acid 3-O-glucuronide (MA-3-GlcA) (Compound 6), Compound 7, Compound 8, Compound 9, Compound 10, Compound 11, glycyrrhizin (Compound 14), Glycyrrhetinic acid 3-O-monoglucuronide (compound 15), bayogenin (Compound 25), bayogenin-hexA-hex-hex (Compound 31), serjanic acid (Compound 26), serjanic acid-hexA-hex (Compound 32), soyasapogenol A (Compound 29), soyasapogenol B (Compound 30), soyasapogenol A-hexA-hex-pent (Compound 34), soyasaponin VI (Compound 35), soyasaponin I (Compound 36), betavulgaroside IV (Compound 33), hederagenin-3GlcA, gypsogenin-3GlcA, gypsogenic acid-3GlcA, or a QS-21 adjuvant, or any combination thereof.

In some embodiments, a genetically modified cell, for example a plant cell or a yeast or an algal cell or an insect cell or a bacterium, or in a genetically modified plant or part thereof, comprises an increased content of Compound 11. In some embodiments, a genetically modified cell, for example a plant cell or a yeast or an algal cell or an insect cell or a bacterium, or in a genetically modified plant or part thereof, comprises an increased content of medicagenic acid 3-O-glucuronide (MA-3-GlcA) (Compound 6). In some embodiments, a genetically modified cell, for example a plant cell or a yeast or an algal cell or an insect cell or a bacterium, or in a genetically modified plant or part thereof, comprises an increased content of Compound 7. In some embodiments, a genetically modified cell, for example a plant cell or a yeast or an algal cell or an insect cell or a bacterium, or in a genetically modified plant or part thereof, comprises an increased content of Compound 8. In some embodiments, a genetically modified cell, for example a plant cell or a yeast or an algal cell or an insect cell or a bacterium, or in a genetically modified plant or part thereof, comprises an increased content of Compound 9. In some embodiments, a genetically modified cell, for example a plant cell or a yeast or an algal cell or an insect cell or a bacterium, or in a genetically modified plant or part thereof, comprises an increased content of Compound 10. In some embodiments, a genetically modified cell, for example a plant cell or a yeast or an algal cell or an insect cell or a bacterium, or in a genetically modified plant or part thereof, comprises an increased content of glycyrrhizin (Compound 14). In some embodiments, a genetically modified cell, for example a plant cell or a yeast or an algal cell or an insect cell or a bacterium, or in a genetically modified plant or part thereof, comprises an increased content of Glycyrrhetinic acid 3-O-monoglucuronide (compound 15). In some embodiments, a genetically modified cell, for example a plant cell or a yeast or an algal cell or an insect cell or a bacterium, or in a genetically modified plant or part thereof, comprises an increased content of bayogenin (Compound 25). In some embodiments, a genetically modified cell, for example a plant cell or a yeast or an algal cell or an insect cell or a bacterium, or in a genetically modified plant or part thereof, comprises an increased content of bayogenin-hexA-hex-hex (Compound 31). In some embodiments, a genetically modified cell, for example a plant cell or a yeast or an algal cell or an insect cell or a bacterium, or in a genetically modified plant or part thereof, comprises an increased content of serjanic acid (Compound 26). In some embodiments, a genetically modified cell, for example a plant cell or a yeast or an algal cell or an insect cell or a bacterium, or in a genetically modified plant or part thereof, comprises an increased content of serjanic acid-hexA-hex (Compound 32). In some embodiments, a genetically modified cell, for example a plant cell or a yeast or an algal cell or an insect cell or a bacterium, or in a genetically modified plant or part thereof, comprises an increased content of soyasapogenol A (Compound 29). In some embodiments, a genetically modified cell, for example a plant cell or a yeast or an algal cell or an insect cell or a bacterium, or in a genetically modified plant or part thereof, comprises an increased content of soyasapogenol B (Compound 30). In some embodiments, a genetically modified cell, for example a plant cell or a yeast or an algal cell or an insect cell or a bacterium, or in a genetically modified plant or part thereof, comprises an increased content of soyasapogenol A-hexA-hex-pent (Compound 34). In some embodiments, a genetically modified cell, for example a plant cell or a yeast or an algal cell or an insect cell or a bacterium, or in a genetically modified plant or part thereof, comprises an increased content of soyasaponin VI (Compound 35). In some embodiments, a genetically modified cell, for example a plant cell or a yeast or an algal cell or an insect cell or a bacterium, or in a genetically modified plant or part thereof, comprises an increased content of soyasaponin I (Compound 36). In some embodiments, a genetically modified cell, for example a plant cell or a yeast or an algal cell or an insect cell or a bacterium, or in a genetically modified plant or part thereof, comprises an increased content of betavulgaroside IV (Compound 33). In some embodiments, a genetically modified cell, for example a plant cell or a yeast or an algal cell or an insect cell or a bacterium, or in a genetically modified plant or part thereof, comprises an increased content of hederagenin-3GlcA. In some embodiments, a genetically modified cell, for example a plant cell or a yeast or an algal cell or an insect cell or a bacterium, or in a genetically modified plant or part thereof, comprises an increased content of gypsogenin-3GlcA. In some embodiments, a genetically modified cell, for example a plant cell or a yeast or an algal cell or an insect cell or a bacterium, or in a genetically modified plant or part thereof, comprises an increased content of gypsogenic acid-3GlcA. In some embodiments, a genetically modified cell, for example a plant cell or a yeast or an algal cell or an insect cell or a bacterium, or in a genetically modified plant or part thereof, comprises an increased content of QS-21 adjuvant. A skilled artisan would appreciate that in some embodiments, instances wherein the content of one triterpenoid saponin is increased additional triterpenoid saponins, or intermediates, or a combination thereof may also be increased in the same time cell.

In some embodiments, a genetically modified cell, for example a plant cell or a yeast or an algal cell, or a genetically modified plant or part thereof, comprises an increased content of a biosynthetic intermediate of a triterpenoid saponin, said intermediate comprising Compound 1, Compound 2, medicagenic acid (Compound 5), oleanolic acid (Compound 3), oleanolic acid-3GlcA, augustic acid (Compound 4), augustic acid-3GlcA, or glycyrrhetinic acid (Compound 13), or any combination thereof. In some embodiments, a genetically modified cell, for example a plant cell or a yeast or an algal cell or an insect cell or a bacterium, or a genetically modified plant or part thereof, comprises an increased content of at least two biosynthetic intermediates of a triterpenoid saponin or of triterpenoid saponins, said intermediates comprising Compound 1, Compound 2, medicagenic acid (Compound 5), oleanolic acid (Compound 3), oleanolic acid-3GlcA, augustic acid (Compound 4), augustic acid-3GlcA, or glycyrrhetinic acid (Compound 13), or any combination thereof, or any combination thereof.

In some embodiments, a genetically modified cell, for example a plant cell or a yeast or an algal cell or an insect cell or a bacterium, or a genetically modified plant or part thereof, comprises an increased content of a biosynthetic intermediate of a triterpenoid saponin, said intermediate comprising medicagenic acid (Compound 5). In some embodiments, a genetically modified cell, for example a plant cell or a yeast or an algal cell or an insect cell or a bacterium, or a genetically modified plant or part thereof, comprises an increased content of a biosynthetic intermediate of a triterpenoid saponin, said intermediate comprising Compound 1. In some embodiments, a genetically modified cell, for example a plant cell or a yeast or an algal cell or an insect cell or a bacterium, or a genetically modified plant or part thereof, comprises an increased content of a biosynthetic intermediate of a triterpenoid saponin, said intermediate comprising Compound 2. In some embodiments, a genetically modified cell, for example a plant cell or a yeast or an algal cell or an insect cell or a bacterium, or a genetically modified plant or part thereof, comprises an increased content of a biosynthetic intermediate of a triterpenoid saponin, said intermediate comprising oleanolic acid (Compound 3). In some embodiments, a genetically modified cell, for example a plant cell or a yeast or an algal cell or an insect cell or a bacterium, or a genetically modified plant or part thereof, comprises an increased content of a biosynthetic intermediate of a triterpenoid saponin, said intermediate comprising oleanolic acid-3GlcA. In some embodiments, a genetically modified cell, for example a plant cell or a yeast or an algal cell or an insect cell or a bacterium, or a genetically modified plant or part thereof, comprises an increased content of a biosynthetic intermediate of a triterpenoid saponin, said intermediate comprising augustic acid (Compound 4). In some embodiments, a genetically modified cell, for example a plant cell or a yeast or an algal cell or an insect cell or a bacterium, or a genetically modified plant or part thereof, comprises an increased content of a biosynthetic intermediate of a triterpenoid saponin, said intermediate comprising augustic acid-3GlcA. In some embodiments, a genetically modified cell, for example a plant cell or a yeast or an algal cell or an insect cell or a bacterium, or a genetically modified plant or part thereof, comprises an increased content of a biosynthetic intermediate of a triterpenoid saponin, said intermediate comprising glycyrrhetinic acid (Compound 13), or any combination thereof.

In some embodiments, a triterpenoid saponin having decreased content in a plant or plant part comprising at least one genetically modified cell, comprises a compound having a bitter taste or a toxin. In some embodiments, a triterpenoid saponin having decreased content comprises triterpenoid saponin selected from medicagenic acid 3-O-glucuronide (MA-3-GlcA) (Compound 6), Compound 7, Compound 8, Compound 9, Compound 10, Compound 11, glycyrrhizin (Compound 14), Glycyrrhetinic acid 3-O-monoglucuronide (compound 15), bayogenin (Compound 25), bayogenin-hexA-hex-hex (Compound 31), serjanic acid (Compound 26), serjanic acid-hexA-hex (Compound 32), soyasapogenol A (Compound 29), soyasapogenol B (Compound 30), soyasapogenol A-hexA-hex-pent (Compound 34), soyasaponin VI (Compound 35), soyasaponin I (Compound 36), betavulgaroside IV (Compound 33), hederagenin-3GlcA, gypsogenin-3GlcA, gypsogenic acid-3GlcA, or a QS-21 adjuvant, or any combination thereof. In some embodiments, a triterpenoid saponin having decreased content comprises at least 1, 2, 3, 4, 5, 6, or more triterpenoid saponin selected from medicagenic acid 3-O-glucuronide (MA-3-GlcA) (Compound 6), Compound 7, Compound 8, Compound 9, Compound 10, Compound 11, glycyrrhizin (Compound 14), Glycyrrhetinic acid 3-O-monoglucuronide (compound 15), bayogenin (Compound 25), bayogenin-hexA-hex-hex (Compound 31), serjanic acid (Compound 26), serjanic acid-hexA-hex (Compound 32), soyasapogenol A (Compound 29), soyasapogenol B (Compound 30), soyasapogenol A-hexA-hex-pent (Compound 34), soyasaponin VI (Compound 35), soyasaponin I (Compound 36), betavulgaroside IV (Compound 33), hederagenin-3GlcA, gypsogenin-3GlcA, gypsogenic acid-3GlcA, or a QS-21 adjuvant, or any combination thereof.

In some embodiments, a triterpenoid saponin having decreased content comprises at least 1, 2, 3, 4, 5, 6, or more triterpenoid saponin selected from medicagenic acid 3-O-glucuronide (MA-3-GlcA) (Compound 6), Compound 7, Compound 8, Compound 9, Compound 10, Compound 11, glycyrrhizin (Compound 14), Glycyrrhetinic acid 3-O-monoglucuronide (compound 15), bayogenin (Compound 25), bayogenin-hexA-hex-hex (Compound 31), serjanic acid (Compound 26), serjanic acid-hexA-hex (Compound 32), soyasapogenol A (Compound 29), soyasapogenol B (Compound 30), soyasapogenol A-hexA-hex-pent (Compound 34), soyasaponin VI (Compound 35), soyasaponin I (Compound 36), betavulgaroside IV (Compound 33), hederagenin-3GlcA, gypsogenin-3GlcA, gypsogenic acid-3GlcA, or a QS-21 adjuvant, or any combination thereof.

In some embodiments, a triterpenoid saponin having decreased content in a genetically modified plant or part thereof comprising at least one genetically modified cell, comprises triterpenoid saponin selected from medicagenic acid 3-O-glucuronide (MA-3-GlcA) (Compound 6), Compound 7, Compound 8, Compound 9, Compound 10, Compound 11, glycyrrhizin (Compound 14), Glycyrrhetinic acid 3-O-monoglucuronide (compound 15), bayogenin (Compound 25), bayogenin-hexA-hex-hex (Compound 31), serjanic acid (Compound 26), serjanic acid-hexA-hex (Compound 32), soyasapogenol A (Compound 29), soyasapogenol B (Compound 30), soyasapogenol A-hexA-hex-pent (Compound 34), soyasaponin VI (Compound 35), soyasaponin I (Compound 35), betavulgaroside IV (Compound 33), hederagenin-3GlcA, gypsogenin-3GlcA, gypsogenic acid-3GlcA, or a QS-21 adjuvant, or any combination thereof.

In some embodiments, a genetically modified plant or part thereof comprising at least one genetically modified plant cell comprises a decreased content of Compound 11. In some embodiments, a genetically modified plant or part thereof comprising at least one genetically modified plant cell, comprises a decreased content of medicagenic acid 3-O-glucuronide (MA-3-GlcA) (Compound 6). In some embodiments, a genetically modified plant or part thereof comprising at least one genetically modified plant cell, comprises a decreased content of Compound 7. In some embodiments, a genetically modified plant or part thereof comprising at least one genetically modified plant cell, comprises a decreased content of Compound 8. In some embodiments, a genetically modified plant or part thereof comprising at least one genetically modified plant cell, comprises a decreased content of Compound 9. In some embodiments, a genetically modified plant or part thereof comprising at least one genetically modified plant cell, comprises a decreased content of Compound 10. In some embodiments, a genetically modified plant or part thereof comprising at least one genetically modified plant cell, comprises a decreased content of glycyrrhizin (Compound 14). In some embodiments, a genetically modified plant or part thereof comprising at least one genetically modified plant cell, comprises a decreased content of Glycyrrhetinic acid 3-O-monoglucuronide (compound 15). In some embodiments, a genetically modified plant or part thereof comprising at least one genetically modified plant cell, comprises a decreased content of bayogenin (Compound 25). In some embodiments, a genetically modified plant or part thereof comprising at least one genetically modified plant cell, comprises a decreased content of bayogenin-hexA-hex-hex (Compound 31). In some embodiments, a genetically modified plant or part thereof comprising at least one genetically modified plant cell, comprises a decreased content of serjanic acid (Compound 26). In some embodiments, a genetically modified plant or part thereof comprising at least one genetically modified plant cell, comprises a decreased content of serjanic acid-hexA-hex (Compound 32). In some embodiments, a genetically modified plant or part thereof comprising at least one genetically modified plant cell, comprises a decreased content of soyasapogenol A (Compound 29). In some embodiments, a genetically modified plant or part thereof comprising at least one genetically modified plant cell, comprises a decreased content of soyasapogenol B (Compound 30). In some embodiments, a genetically modified plant or part thereof comprising at least one genetically modified plant cell, comprises a decreased content of soyasapogenol A-hexA-hex-pent (Compound 34). In some embodiments, a genetically modified plant or part thereof comprising at least one genetically modified plant cell, comprises a decreased content of soyasaponin VI (Compound 35). In some embodiments, a genetically modified plant or part thereof comprising at least one genetically modified plant cell, comprises a decreased content of soyasaponin I (Compound 36). In some embodiments, a genetically modified plant or part thereof comprising at least one genetically modified plant cell, comprises a decreased content of betavulgaroside IV (Compound 33). In some embodiments, a genetically modified plant or part thereof comprising at least one genetically modified plant cell, comprises a decreased content of hederagenin-3GlcA. In some embodiments, a genetically modified plant or part thereof comprising at least one genetically modified plant cell, comprises a decreased content of gypsogenin-3GlcA. In some embodiments, a genetically modified plant or part thereof comprising at least one genetically modified plant cell, comprises a decreased content of gypsogenic acid-3GlcA. In some embodiments, a genetically modified plant or part thereof comprising at least one genetically modified plant cell, comprises a decreased content of QS-21 adjuvant. A skilled artisan would appreciate that in some embodiments, instances wherein the content of one triterpenoid saponin is decreased additional triterpenoid saponins, or intermediates, or a combination thereof may also be decreased in the same time cell.

In some embodiments, a genetically modified plant or part thereof comprising at least one genetically modified plant cell, comprises a decreased content of a biosynthetic intermediate of a triterpenoid saponin, said intermediate comprising Compound 1, Compound 2, medicagenic acid (Compound 5), oleanolic acid (Compound 3), oleanolic acid-3GlcA, augustic acid (Compound 4), augustic acid-3GlcA, or glycyrrhetinic acid (Compound 13), or any combination thereof. In some embodiments, a genetically modified plant or part thereof comprising at least one genetically modified plant cell, comprises a decreased content of at least two biosynthetic intermediates of a triterpenoid saponin or of triterpenoid saponins, said intermediates comprising Compound 1, Compound 2, medicagenic acid (Compound 5), oleanolic acid (Compound 3), oleanolic acid-3GlcA, augustic acid (Compound 4), augustic acid-3GlcA, or glycyrrhetinic acid (Compound 13), or any combination thereof, or any combination thereof.

In some embodiments, a genetically modified plant or part thereof comprising at least one genetically modified plant cell, comprises a decreased content of a biosynthetic intermediate of a triterpenoid saponin, said intermediate comprising medicagenic acid (Compound 5). In some embodiments, a genetically modified plant or part thereof comprising at least one genetically modified plant cell, comprises a decreased content of a biosynthetic intermediate of a triterpenoid saponin, said intermediate comprising Compound 1. In some embodiments, a genetically modified plant or part thereof comprising at least one genetically modified plant cell, comprises a decreased content of a biosynthetic intermediate of a triterpenoid saponin, said intermediate comprising Compound 2. In some embodiments, a genetically modified plant or part thereof comprising at least one genetically modified plant cell, comprises a decreased content of a biosynthetic intermediate of a triterpenoid saponin, said intermediate comprising oleanolic acid (Compound 3). In some embodiments, a genetically modified plant or part thereof comprising at least one genetically modified plant cell, comprises a decreased content of a biosynthetic intermediate of a triterpenoid saponin, said intermediate comprising oleanolic acid-3GlcA. In some embodiments, a genetically modified plant or part thereof comprising at least one genetically modified plant cell, comprises a decreased content of a biosynthetic intermediate of a triterpenoid saponin, said intermediate comprising augustic acid (Compound 4). In some embodiments, a genetically modified plant or part thereof comprising at least one genetically modified plant cell, comprises a decreased content of a biosynthetic intermediate of a triterpenoid saponin, said intermediate comprising augustic acid-3GlcA. In some embodiments, a genetically modified plant or part thereof comprising at least one genetically modified plant cell, comprises a decreased content of a biosynthetic intermediate of a triterpenoid saponin, said intermediate comprising glycyrrhetinic acid (Compound 13), or any combination thereof.

In some embodiments, a genetically modified cell disclosed herein comprises an increased content of at least one triterpenoid saponin, a derivative thereof, a metabolite thereof, or a biosynthetic intermediate thereof, and an altered content of a phytosterol, or a derivative thereof, a metabolite thereof, or a biosynthetic intermediate thereof, a cholesterol, or a derivative thereof, a metabolite thereof, or a biosynthetic intermediate thereof, a phytocholesterol, or a derivative thereof, a metabolite thereof, or a biosynthetic intermediate thereof; a cholestenol, or a derivative thereof, a metabolite thereof, or a biosynthetic intermediate thereof, or a phytocholestenol, or a derivative thereof, a metabolite thereof, or a biosynthetic intermediate thereof.

In some embodiments, a genetically modified cell disclosed herein comprises an increased content of at least one triterpenoid saponin, a derivative thereof, a metabolite thereof, or a biosynthetic intermediate thereof, and a reduced content of a phytosterol, or a derivative thereof, a metabolite thereof, or a biosynthetic intermediate thereof, a cholesterol, or a derivative thereof, a metabolite thereof, or a biosynthetic intermediate thereof, a phytocholesterol, or a derivative thereof, a metabolite thereof, or a biosynthetic intermediate thereof; a cholestenol, or a derivative thereof, a metabolite thereof, or a biosynthetic intermediate thereof, or a phytocholestenol, or a derivative thereof, a metabolite thereof, or a biosynthetic intermediate thereof.

In some embodiments, a genetically modified cell disclosed herein comprises a decreased content of at least one triterpenoid saponin, a derivative thereof, a metabolite thereof, or a biosynthetic intermediate thereof, and an altered content of a phytosterol, or a derivative thereof, a metabolite thereof, or a biosynthetic intermediate thereof, a cholesterol, or a derivative thereof, a metabolite thereof, or a biosynthetic intermediate thereof, a phytocholesterol, or a derivative thereof, a metabolite thereof, or a biosynthetic intermediate thereof; a cholestenol, or a derivative thereof, a metabolite thereof, or a biosynthetic intermediate thereof, or a phytocholestenol, or a derivative thereof, a metabolite thereof, or a biosynthetic intermediate thereof.

In some embodiments, a genetically modified cell disclosed herein comprises a decreased content of at least one triterpenoid saponin, a derivative thereof, a metabolite thereof, or a biosynthetic intermediate thereof, and an increased content of a phytosterol, or a derivative thereof, a metabolite thereof, or a biosynthetic intermediate thereof, a cholesterol, or a derivative thereof, a metabolite thereof, or a biosynthetic intermediate thereof, a phytocholesterol, or a derivative thereof, a metabolite thereof, or a biosynthetic intermediate thereof; a cholestenol, or a derivative thereof, a metabolite thereof, or a biosynthetic intermediate thereof, or a phytocholestenol, or a derivative thereof, a metabolite thereof, or a biosynthetic intermediate thereof.

In some embodiments, a genetically modified plant disclosed herein comprises an increased content of at least one triterpenoid saponin, a derivative thereof, a metabolite thereof, or a biosynthetic intermediate thereof, and an altered content of a phytosterol, or a derivative thereof, a metabolite thereof, or a biosynthetic intermediate thereof, a cholesterol, or a derivative thereof, a metabolite thereof, or a biosynthetic intermediate thereof, a phytocholesterol, or a derivative thereof, a metabolite thereof, or a biosynthetic intermediate thereof; a cholestenol, or a derivative thereof, a metabolite thereof, or a biosynthetic intermediate thereof, or a phytocholestenol, or a derivative thereof, a metabolite thereof, or a biosynthetic intermediate thereof.

In some embodiments, a genetically modified plant disclosed herein comprises an increased content of at least one triterpenoid saponin, a derivative thereof, a metabolite thereof, or a biosynthetic intermediate thereof, and a decreased content of a phytosterol, or a derivative thereof, a metabolite thereof, or a biosynthetic intermediate thereof; a cholesterol, or a derivative thereof, a metabolite thereof, or a biosynthetic intermediate thereof; a phytocholesterol, or a derivative thereof, a metabolite thereof, or a biosynthetic intermediate thereof; a cholestenol, or a derivative thereof, a metabolite thereof, or a biosynthetic intermediate thereof; or a phytocholestenol, or a derivative thereof, a metabolite thereof, or a biosynthetic intermediate thereof.

In some embodiments, a genetically modified plant disclosed herein comprises a decreased content of at least one triterpenoid saponin, a derivative thereof, a metabolite thereof, or a biosynthetic intermediate thereof and an altered content of a phytosterol, or a derivative thereof, a metabolite thereof, or a biosynthetic intermediate thereof; a cholesterol, or a derivative thereof, a metabolite thereof, or a biosynthetic intermediate thereof; a phytocholesterol, or a derivative thereof, a metabolite thereof, or a biosynthetic intermediate thereof; a cholestenol, or a derivative thereof, a metabolite thereof, or a biosynthetic intermediate thereof; or a phytocholestenol, or a derivative thereof, a metabolite thereof, or a biosynthetic intermediate thereof.

In some embodiments, a genetically modified plant disclosed herein comprises a decreased content of at least one triterpenoid saponin, a derivative thereof, a metabolite thereof, or a biosynthetic intermediate thereof and an increased content of a phytosterol, or a derivative thereof, a metabolite thereof, or a biosynthetic intermediate thereof; a cholesterol, or a derivative thereof, a metabolite thereof, or a biosynthetic intermediate thereof; a phytocholesterol, or a derivative thereof, a metabolite thereof, or a biosynthetic intermediate thereof; a cholestenol, or a derivative thereof, a metabolite thereof, or a biosynthetic intermediate thereof; or a phytocholestenol, or a derivative thereof, a metabolite thereof, or a biosynthetic intermediate thereof.

In some embodiments, a method of producing at least one triterpenoid saponin, a derivative thereof, a metabolite thereof, or a biosynthetic intermediate thereof, in a genetically modified cell or a genetically modified plant comprises altering the content of at least one triterpenoid saponin, a derivative thereof, a metabolite thereof, or a biosynthetic intermediate thereof, compared to an unmodified cell or an unmodified plant and decreasing the content of a phytosterol, or a derivative thereof, a metabolite thereof, or a biosynthetic intermediate thereof; a cholesterol, or a derivative thereof, a metabolite thereof, or a biosynthetic intermediate thereof; a phytocholesterol, or a derivative thereof, a metabolite thereof, or a biosynthetic intermediate thereof; a cholestenol, or a derivative thereof, a metabolite thereof, or a biosynthetic intermediate thereof; or a phytocholestenol, or a derivative thereof, a metabolite thereof, or a biosynthetic intermediate thereof.

In some embodiments, a method of producing at least one triterpenoid saponin, a derivative thereof, a metabolite thereof, or a biosynthetic intermediate thereof, in a genetically modified cell or a genetically modified plant comprises increasing the content of at least one triterpenoid saponin, a derivative thereof, a metabolite thereof, or a biosynthetic intermediate thereof, compared to an unmodified cell or an unmodified plant and decreasing the content of a phytosterol, or a derivative thereof, a metabolite thereof, or a biosynthetic intermediate thereof, a cholesterol, or a derivative thereof, a metabolite thereof, or a biosynthetic intermediate thereof, a phytocholesterol, or a derivative thereof, a metabolite thereof, or a biosynthetic intermediate thereof; a cholestenol, or a derivative thereof, a metabolite thereof, or a biosynthetic intermediate thereof, or a phytocholestenol, or a derivative thereof, a metabolite thereof, or a biosynthetic intermediate thereof.

In some embodiments, a method of reducing the content of at least one triterpenoid saponin, a derivative thereof, a metabolite thereof, or a biosynthetic intermediate thereof, comprises genetically modifying at least one plant cell or at least one cell of a plant or plant part, said genetically modified plant cell comprising a reduced content of at least one triterpenoid saponin, a derivative thereof, a metabolite thereof, or a biosynthetic intermediate thereof, and an altered content of a phytosterol, or a derivative thereof, a metabolite thereof, or a biosynthetic intermediate thereof, a cholesterol, or a derivative thereof, a metabolite thereof, or a biosynthetic intermediate thereof; a phytocholesterol, or a derivative thereof, a metabolite thereof, or a biosynthetic intermediate thereof; a cholestenol, or a derivative thereof, a metabolite thereof, or a biosynthetic intermediate thereof, or a phytocholestenol, or a derivative thereof, a metabolite thereof, or a biosynthetic intermediate thereof.

In some embodiments, a method of reducing the content of at least one triterpenoid saponin, a derivative thereof, a metabolite thereof, or a biosynthetic intermediate thereof, comprises genetically modifying at least one plant cell or at least one cell of a plant or plant part, said genetically modified plant cell comprising a reduced content of at least one triterpenoid saponin, a derivative thereof, a metabolite thereof, or a biosynthetic intermediate thereof, and an increased content of a phytosterol, or a derivative thereof, a metabolite thereof, or a biosynthetic intermediate thereof, a cholesterol, or a derivative thereof, a metabolite thereof, or a biosynthetic intermediate thereof; a phytocholesterol, or a derivative thereof, a metabolite thereof, or a biosynthetic intermediate thereof; a cholestenol, or a derivative thereof, a metabolite thereof, or a biosynthetic intermediate thereof, or a phytocholestenol, or a derivative thereof, a metabolite thereof, or a biosynthetic intermediate thereof.

In some embodiments, a method of increasing the content of at least one triterpenoid saponin, a derivative thereof, a metabolite thereof, or a biosynthetic intermediate thereof, comprises genetically modifying at least one plant cell or at least one cell of a plant or plant part, said genetically modified plant cell comprising an increased content of at least one triterpenoid saponin, a derivative thereof, a metabolite thereof, or a biosynthetic intermediate thereof, and an altered content of a phytosterol, or a derivative thereof, a metabolite thereof, or a biosynthetic intermediate thereof, a cholesterol, or a derivative thereof, a metabolite thereof, or a biosynthetic intermediate thereof, a phytocholesterol, or a derivative thereof, a metabolite thereof, or a biosynthetic intermediate thereof, a cholestenol, or a derivative thereof, a metabolite thereof, or a biosynthetic intermediate thereof, or a phytocholestenol, or a derivative thereof, a metabolite thereof, or a biosynthetic intermediate thereof.

In some embodiments, a method of increasing the content of at least one triterpenoid saponin, a derivative thereof, a metabolite thereof, or a biosynthetic intermediate thereof, comprises genetically modifying at least one plant cell or at least one cell of a plant or plant part, said genetically modified plant cell comprising an increased content of at least one triterpenoid saponin, a derivative thereof, a metabolite thereof, or a biosynthetic intermediate thereof, and a reduced content of a phytosterol, or a derivative thereof, a metabolite thereof, or a biosynthetic intermediate thereof, a cholesterol, or a derivative thereof, a metabolite thereof, or a biosynthetic intermediate thereof, a phytocholesterol, or a derivative thereof, a metabolite thereof, or a biosynthetic intermediate thereof, a cholestenol, or a derivative thereof, a metabolite thereof, or a biosynthetic intermediate thereof, or a phytocholestenol, or a derivative thereof, a metabolite thereof, or a biosynthetic intermediate thereof.

### Plants, Plant Parts, and Cells

According to some embodiments, the cell or organism described herein, comprising the at least one heterologous gene encoding an enzyme has an elevated content of at least one steroidal alkaloid, derivative thereof, metabolite thereof, or biosynthetic intermediate thereof; at least one steroidal saponin, derivative thereof, metabolite thereof, or biosynthetic intermediate thereof; or at least one triterpenoid saponin, derivative thereof, metabolite thereof, or biosynthetic intermediate thereof, compared to a corresponding non-genetically modified cell or non-transgenic plant, respectively. In other embodiments, the genetically modified plant or plant part comprising an at least one genetically modified cell described herein, comprising an at least one modified endogenous gene encoding an enzyme of the steroidal alkaloid pathway, the steroidal saponin pathway, or the triterpenoid synthetic pathway has an elevated content of at least one steroidal alkaloid, derivative thereof, metabolite thereof, or biosynthetic intermediate thereof; at least one steroidal saponin, derivative thereof, metabolite thereof, or biosynthetic intermediate thereof; at least one triterpenoid saponin, derivative thereof, metabolite thereof, or biosynthetic intermediate thereof, compared to a corresponding non-genetically modified plant. In other embodiments, the genetically modified plant or plant part comprising an at least one genetically modified cell described herein, comprising an at least one heterologous gene encoding an enzyme of the steroidal alkaloid synthetic pathway, the steroidal saponin synthetic pathway, or the triterpenoid synthetic pathway has an elevated content of at least one steroidal alkaloid, derivative thereof, metabolite thereof, or biosynthetic intermediate thereof, at least one steroidal saponin, derivative thereof, metabolite thereof, or biosynthetic intermediate thereof; at least one triterpenoid saponin, derivative thereof, metabolite thereof, or biosynthetic intermediate thereof, compared to a corresponding non-genetically modified plant. In other embodiments, the genetically modified plant or plant part comprising an at least one genetically modified cell described herein, comprising the at least one modified endogenous gene encoding an enzyme of the steroidal alkaloid synthetic pathway, the steroidal saponin synthetic pathway, or the triterpenoid synthetic pathway has a reduced content of at least one steroidal alkaloid, derivative thereof, metabolite thereof, or biosynthetic intermediate thereof; at least one steroidal saponin, derivative thereof, metabolite thereof, or biosynthetic intermediate thereof; at least one triterpenoid saponin, derivative thereof, metabolite thereof, or biosynthetic intermediate thereof, compared to a corresponding non-genetically modified plant. As described throughout, the skilled artisan would appreciate that modifications of endogenous genes include but are not limited to increasing expression, decreasing expressing, or mutating the gene, or a combination thereof as described in detail herein.

According to some embodiments, the cell or organism described herein, comprising the at least one heterologous CSLG gene encoding a CSLG enzyme has an elevated content of at least one steroidal alkaloid, derivative thereof, metabolite thereof, or biosynthetic intermediate thereof; at least one steroidal saponin, derivative thereof, metabolite thereof, or biosynthetic intermediate thereof; or at least one triterpenoid saponin, derivative thereof, metabolite thereof, or biosynthetic intermediate thereof, compared to a corresponding non-genetically modified cell or non-transgenic plant, respectively. In other embodiments, the genetically modified plant or plant part comprising an at least one genetically modified cell described herein, comprising an at least one modified endogenous CSLG gene encoding a CSLG enzyme of the steroidal alkaloid synthetic pathway, the steroidal saponin synthetic pathway, or the triterpenoid synthetic pathway has an elevated content of at least one steroidal alkaloid, derivative thereof, metabolite thereof, or biosynthetic intermediate thereof; at least one steroidal saponin, derivative thereof, metabolite thereof, or biosynthetic intermediate thereof, or at least one triterpenoid saponin, derivative thereof, metabolite thereof, or biosynthetic intermediate thereof compared to a corresponding non-genetically modified plant. In other embodiments, the genetically modified plant or plant part comprising an at least one genetically modified cell described herein, comprising an at least one heterologous CSLG gene encoding an enzyme of the steroidal alkaloid synthetic pathway, the steroidal saponin synthetic pathway, or the triterpenoid synthetic pathway has an elevated content of at least one steroidal alkaloid, derivative thereof, metabolite thereof, or biosynthetic intermediate thereof, at least one steroidal saponin, derivative thereof, metabolite thereof, or biosynthetic intermediate thereof; or at least one triterpenoid saponin, derivative thereof, metabolite thereof, or biosynthetic intermediate thereof compared to a corresponding non-genetically modified plant. In other embodiments, the genetically modified plant or plant part comprising an at least one genetically modified cell described herein, comprising the at least one modified endogenous CSLG gene encoding an enzyme of the steroidal alkaloid synthetic pathway, the steroidal saponin synthetic pathway, or the triterpenoid synthetic pathway has a reduced content of at least one steroidal alkaloid, derivative thereof, metabolite thereof, or biosynthetic intermediate thereof, at least one steroidal saponin, derivative thereof, metabolite thereof, or biosynthetic intermediate thereof, or at least one triterpenoid saponin, derivative thereof, metabolite thereof, or biosynthetic intermediate thereof compared to a corresponding non-genetically modified plant.

In some embodiments, derivatives of steroidal alkaloids, steroidal saponins, or triterpenoid saponins comprise glycosylated derivatives of, respectively, steroidal alkaloids, steroidal saponins, or triterpenoid saponins.

In some embodiments, a cell or plant species or plant part that does not normally express for example at least a saponin beta-amyrin synthase, a cytochrome P450, a glycosyltransferase, an acyltransferase, a glucuronosyltransferase, or a CSLG and/or does not normally produce a particular steroidal alkaloid, a derivative thereof, a metabolite thereof, or a biosynthetic intermediate thereof, a particular steroidal saponin, a derivative thereof, a metabolite thereof, or a biosynthetic intermediate thereof, or a particular triterpenoid saponin, a derivative thereof, a metabolite thereof, or a biosynthetic intermediate thereof, is genetically modified to express a saponin beta-amyrin synthase, a cytochrome P450, a glycosyltransferase, an acyltransferase, a glucuronosyltransferase, or a CSLG, or any combination thereof. In some embodiments, a cell or plant species or plant part that does not normally express a saponin beta-amyrin synthase, a cytochrome P450, a glycosyltransferase, an acyltransferase, a glucuronosyltransferase, or a CSLG and/or does not normally produce a particular steroidal alkaloid, a derivative thereof, a metabolite thereof, or a biosynthetic intermediate thereof, a particular steroidal saponin, a derivative thereof, a metabolite thereof, or a biosynthetic intermediate thereof, or a particular triterpenoid saponin, a derivative thereof, a metabolite thereof, or a biosynthetic intermediate thereof, is genetically modified to produce a steroidal alkaloid, a derivative thereof, a metabolite thereof, or a biosynthetic intermediate thereof, a steroidal saponin, a derivative thereof, a metabolite thereof, or a biosynthetic intermediate thereof, or a triterpenoid saponin, a derivative thereof, a metabolite thereof, or a biosynthetic intermediate thereof, or any combination thereof. In other embodiments, a cell or plant species or plant part that normally expresses a given amount of a saponin beta-amyrin synthase, a cytochrome P450, a glycosyltransferase, an acyltransferase, a glucuronosyltransferase, or a CSLG, or any combination thereof is genetically modified to overexpress a saponin beta-amyrin synthase, a cytochrome P450, a glycosyltransferase, an acyltransferase, a glucuronosyltransferase, or a CSLG, or any combination thereof. In other embodiments, a cell or plant species or plant part that normally expresses a given amount of a saponin beta-amyrin synthase, a cytochrome P450, a glycosyltransferase, an acyltransferase, a glucuronosyltransferase, or a CSLG, or any combination thereof is genetically modified to reduce or silence expression of a saponin beta-amyrin synthase, a cytochrome P450, a glycosyltransferase, an acyltransferase, a glucuronosyltransferase, or a CSLG, or any combination thereof.

In other embodiments, a cell or plant species or plant part that normally produces a given amount of a steroidal alkaloid, a derivative thereof, a metabolite thereof, or a biosynthetic intermediate thereof, a steroidal saponin, a derivative thereof, a metabolite thereof, or a biosynthetic intermediate thereof, or a triterpenoid saponin, a derivative thereof, a metabolite thereof, or a biosynthetic intermediate thereof, is genetically modified to overexpress a saponin beta-amyrin synthase, a cytochrome P450, a glycosyltransferase, an acyltransferase, a glucuronosyltransferase, or a CSLG, or any combination thereof leading to an increases production of a steroidal alkaloid, a derivative thereof, a metabolite thereof, or a biosynthetic intermediate thereof, a steroidal saponin, a derivative thereof, a metabolite thereof, or a biosynthetic intermediate thereof, or a triterpenoid saponin, a derivative thereof, a metabolite thereof, or a biosynthetic intermediate thereof, or any combination thereof.

In other embodiments, a cell or plant species or plant part that normally produces a given amount of a steroidal alkaloid, a derivative thereof, a metabolite thereof, or a biosynthetic intermediate thereof, a steroidal saponin, a derivative thereof, a metabolite thereof, or a biosynthetic intermediate thereof, or a triterpenoid saponin, a derivative thereof, a metabolite thereof, or a biosynthetic intermediate thereof, is genetically modified to reduce or silence expression of a saponin beta-amyrin synthase, a cytochrome P450, a glycosyltransferase, an acyltransferase, a glucuronosyltransferase, or a CSLG, or any combination thereof leading to a decreased production of a steroidal alkaloid, a derivative thereof, a metabolite thereof, or a biosynthetic intermediate thereof, a steroidal saponin, a derivative thereof, a metabolite thereof, or a biosynthetic intermediate thereof, a triterpenoid saponin, a derivative thereof, a metabolite thereof, or a biosynthetic intermediate thereof, or any combination thereof.

In some embodiments, disclosed herein are seeds of the genetically modified plant, wherein plants grown from said seeds and expressing at least one saponin beta-amyrin synthase, cytochrome P450, a glycosyltransferase, acyltransferase, a glucuronosyltransferase, or CSLG compared to plants grown from corresponding unmodified, thereby containing expression or overexpression of at least one saponin beta-amyrin synthase, cytochrome P450, a glycosyltransferase, acyltransferase, a glucuronosyltransferase, or CSLG and/or having increased content of a steroidal alkaloid, a derivative thereof, a metabolite thereof, or a biosynthetic intermediate thereof, a steroidal saponin, a derivative thereof, a metabolite thereof, or a biosynthetic intermediate thereof, or a triterpenoid saponin, a derivative thereof, a metabolite thereof, or a biosynthetic intermediate thereof.

The cells disclosed herein comprise numerous varieties, including, but not limited to, yeast, algal, insect, bacterium, and plant cells. In some embodiments, a genetically modified cell comprises a plant cell. In some embodiments, a genetically modified cell comprises a yeast cell. In some embodiments, a genetically modified cell comprises an algal cell. In some embodiments, a genetically modified cell comprises an insect cell. In some embodiments, a genetically modified cell comprises a bacterium.

Generally, most yeast, algae, insect cells, and bacterium do not naturally synthesize steroidal alkaloids, steroidal saponins, or triterpenoid saponins. In some embodiments, in order to produce a steroidal alkaloid, steroidal saponin, or triterpenoid saponin in a yeast or an algal cell or an insect cell or a bacterium, genetic modification of the yeast or algal cell or insect cell or bacterium comprises introduction of an enzyme or enzymes necessary to produce precursors or substrates, or a combination thereof, of the triterpenoid saponin biosynthetic pathway. For example, see Example 14 below wherein the S. cerevisiae were genetically modified to express a heterologous UDP-glucose 6-dehydrogenase I in order to produce the necessary precursors for substrates of the triterpenoid saponin biosynthetic pathway. In some embodiments, a genetically modified cell expresses at least one heterologous gene for the production of precursors, substrates, or a combination thereof, of the steroidal alkaloid biosynthetic pathway, a steroidal saponin biosynthetic pathway, or the triterpenoid biosynthetic pathway. In some embodiments, a genetically modified cell expresses at least one heterologous gene for the production of precursors, substrates, or a combination thereof, of the steroidal alkaloid biosynthetic pathway, the steroidal saponin biosynthetic pathway, or the triterpenoid biosynthetic pathway, and expresses at least one heterologous gene encoding an enzyme of the steroidal alkaloid biosynthetic pathway, the steroidal saponin biosynthetic pathway, or the triterpenoid biosynthetic pathway.

In some embodiments, cells comprise a micro-organism. In some embodiments, the genetically modified cell is a yeast cell. In some embodiments, the yeast is from the Saccharomycetes order. In some embodiments, the yeast is from the Saccharomycetes order and is selected from the group of genera consisting of the Saccharomyces genus (e.g., Saccharomyces cerevisiae), the Schizosaccharomyces genus, the Pichia genus (e.g., Pichia pastoris), the Hansenula genus (e.g., Hansenula polymorpha), the Yarrowia genus (e.g., Yarrowia lipollytica), the Kluyveromyces genus (e.g., Kluyveromyces lactis), and the Candida genus (e.g., Candida albicans, Candida utilis). In some embodiments, a yeast is selected from a Saccharomyces genus, a Schizosaccharomyces genus, a Pichia genus, a Yarrowia genus, a Kluyveromyces genus, or a Candida genus. In some embodiments, a yeast is selected from Saccharomyces cerevisiae or Candida albicans.

In some embodiments, a genetically modified yeast may be utilized in a method of producing a steroidal alkaloid, a steroidal saponin, or a triterpenoid saponin as described herein.

In some embodiments, the genetically modified cell is an algal cell. In some embodiments, the cell is any one of a variety of algae, including, but not limited to, chlorophytes (green algae), rhodophytes (red algae), or phaeo-phytes (brown algae). In some embodiments, the chlorophyte is from the Chlamydomonadales order or the Chlorellales order. In some embodiments, the chlorophyte is from the Chlamydomonadales order and is selected from the group of genera consisting of the Chamydomonas genus (e.g., Chlamydomonas reinhardtii) and the Dunaliella genus. In some embodiments, the chlorophyte is from the Chlorellales order and is selected from the Chlorella genus.

In some embodiments, a genetically modified alga may be utilized in a method of producing a steroidal alkaloid, a steroidal saponin, or a triterpenoid saponin as described herein.

In some embodiments, the genetically modified cell disclosed herein is a bacterial cell. In one embodiment, the bacterial cell is an Escherichia coli cell. In another embodiment, the cell is an Acremonium rutilum, Aspergillus oryzae, Yarrowia lipolytica, Bacillus sp. JPJ, Brevundimonas sp. SGJ, E. herbicola, Citrobacter freundii, Symbiobacterium, or Pseudomonas aeruginosa cell. In another embodiment, the bacterial cell is from a bacterium involved in fermentation of dairy products. In one embodiment, the bacterium is Streptococcus lactis. In another embodiment, the bacterium is a Lactobacillus. In one embodiment, the Lactobacillus is Lactobacillus bulgaricus. In another embodiment, the bacterium is a Lactococcus or a Leuconostoc.

In some embodiments, a genetically modified bacterium may be utilized in a method of producing a steroidal alkaloid, a steroidal saponin, or a triterpenoid saponin as described herein.

In some embodiments, a genetically modified plant disclosed herein comprises an increased content of at least one steroidal alkaloid, a derivative thereof, a metabolite thereof, or a biosynthetic intermediate thereof, at least one steroidal saponin, a derivative thereof, a metabolite thereof, or a biosynthetic intermediate thereof, at least one triterpenoid saponin, a derivative thereof, a metabolite thereof, or a biosynthetic intermediate thereof, and an altered content of a phytosterol, or a derivative thereof, a metabolite thereof, or a biosynthetic intermediate thereof, a cholesterol, or a derivative thereof, a metabolite thereof, or a biosynthetic intermediate thereof, a phytocholesterol, or a derivative thereof, a metabolite thereof, or a biosynthetic intermediate thereof; a cholestenol, or a derivative thereof, a metabolite thereof, or a biosynthetic intermediate thereof, or a phytocholestenol, or a derivative thereof, a metabolite thereof, or a biosynthetic intermediate thereof.

In some embodiments, a genetically modified plant disclosed herein comprises an increased content of at least one steroidal alkaloid, a derivative thereof, a metabolite thereof, or a biosynthetic intermediate thereof, at least one steroidal saponin, a derivative thereof, a metabolite thereof, or a biosynthetic intermediate thereof, at least one triterpenoid saponin, a derivative thereof, a metabolite thereof, or a biosynthetic intermediate thereof, and a decreased content of a phytosterol, or a derivative thereof, a metabolite thereof, or a biosynthetic intermediate thereof, a cholesterol, or a derivative thereof, a metabolite thereof, or a biosynthetic intermediate thereof, a phytocholesterol, or a derivative thereof, a metabolite thereof, or a biosynthetic intermediate thereof; a cholestenol, or a derivative thereof, a metabolite thereof, or a biosynthetic intermediate thereof, or a phytocholestenol, or a derivative thereof, a metabolite thereof, or a biosynthetic intermediate thereof.

In some embodiments, a genetically modified plant disclosed herein comprises a decreased content of at least one steroidal alkaloid, a derivative thereof, a metabolite thereof, or a biosynthetic intermediate thereof, at least one steroidal saponin, a derivative thereof, a metabolite thereof, or a biosynthetic intermediate thereof, at least one triterpenoid saponin, a derivative thereof, a metabolite thereof, or a biosynthetic intermediate thereof and an altered content of a phytosterol, or a derivative thereof, a metabolite thereof, or a biosynthetic intermediate thereof, a cholesterol, or a derivative thereof, a metabolite thereof, or a biosynthetic intermediate thereof, a phytocholesterol, or a derivative thereof, a metabolite thereof, or a biosynthetic intermediate thereof; a cholestenol, or a derivative thereof, a metabolite thereof, or a biosynthetic intermediate thereof, or a phytocholestenol, or a derivative thereof, a metabolite thereof, or a biosynthetic intermediate thereof.

In some embodiments, a genetically modified plant disclosed herein comprises a decreased content of at least one steroidal alkaloid, a derivative thereof, a metabolite thereof, or a biosynthetic intermediate thereof, at least one steroidal saponin, a derivative thereof, a metabolite thereof, or a biosynthetic intermediate thereof, at least one triterpenoid saponin, a derivative thereof, a metabolite thereof, or a biosynthetic intermediate thereof and an increased content of a phytosterol, or a derivative thereof, a metabolite thereof, or a biosynthetic intermediate thereof, a cholesterol, or a derivative thereof, a metabolite thereof, or a biosynthetic intermediate thereof, a phytocholesterol, or a derivative thereof, a metabolite thereof, or a biosynthetic intermediate thereof, a cholestenol, or a derivative thereof, a metabolite thereof, or a biosynthetic intermediate thereof, or a phytocholestenol, or a derivative thereof, a metabolite thereof, or a biosynthetic intermediate thereof.

In some embodiments, a method of producing at least one steroidal alkaloid, a derivative thereof, a metabolite thereof, or a biosynthetic intermediate thereof, at least one steroidal saponin, a derivative thereof, a metabolite thereof, or a biosynthetic intermediate thereof, or at least one triterpenoid saponin, a derivative thereof, a metabolite thereof, or a biosynthetic intermediate thereof, in a genetically modified cell or a genetically modified plant comprises increasing the content of at least one steroidal alkaloid, a derivative thereof, a metabolite thereof, or a biosynthetic intermediate thereof, at least one steroidal saponin, a derivative thereof, a metabolite thereof, or a biosynthetic intermediate thereof, or at least one triterpenoid saponin, a derivative thereof, a metabolite thereof, or a biosynthetic intermediate thereof, compared to an unmodified cell or an unmodified plant and altering the content of a phytosterol, or a derivative thereof, a metabolite thereof, or a biosynthetic intermediate thereof; a cholesterol, or a derivative thereof, a metabolite thereof, or a biosynthetic intermediate thereof, a phytocholesterol, or a derivative thereof, a metabolite thereof, or a biosynthetic intermediate thereof, a cholestenol, or a derivative thereof, a metabolite thereof, or a biosynthetic intermediate thereof, or a phytocholestenol, or a derivative thereof, a metabolite thereof, or a biosynthetic intermediate thereof.

In some embodiments, a method of producing at least one steroidal alkaloid, a derivative thereof, a metabolite thereof, or a biosynthetic intermediate thereof, at least one steroidal saponin, a derivative thereof, a metabolite thereof, or a biosynthetic intermediate thereof; or at least one triterpenoid saponin, a derivative thereof, a metabolite thereof, or a biosynthetic intermediate thereof, in a genetically modified cell or a genetically modified plant comprises increasing the content of at least one steroidal alkaloid, a derivative thereof, a metabolite thereof, or a biosynthetic intermediate thereof, at least one steroidal saponin, a derivative thereof, a metabolite thereof, or a biosynthetic intermediate thereof, or at least one triterpenoid saponin, a derivative thereof, a metabolite thereof, or a biosynthetic intermediate thereof, compared to an unmodified cell or an unmodified plant and decreasing the content of a phytosterol, or a derivative thereof, a metabolite thereof, or a biosynthetic intermediate thereof; a cholesterol, or a derivative thereof, a metabolite thereof, or a biosynthetic intermediate thereof, a phytocholesterol, or a derivative thereof, a metabolite thereof, or a biosynthetic intermediate thereof, a cholestenol, or a derivative thereof, a metabolite thereof, or a biosynthetic intermediate thereof, or a phytocholestenol, or a derivative thereof, a metabolite thereof, or a biosynthetic intermediate thereof.

In some embodiments, a method of reducing the content of at least one steroidal alkaloid, a derivative thereof, a metabolite thereof, or a biosynthetic intermediate thereof, at least one steroidal saponin, a derivative thereof, a metabolite thereof, or a biosynthetic intermediate thereof, or at least one triterpenoid saponin, a derivative thereof, a metabolite thereof, or a biosynthetic intermediate thereof, comprises genetically modifying at least one plant cell or at least one cell of a plant or plant part, said genetically modified plant cell comprising a reduced content of at least one steroidal alkaloid, a derivative thereof, a metabolite thereof, at least one steroidal saponin, a derivative thereof, a metabolite thereof, or a biosynthetic intermediate thereof, or at least one triterpenoid saponin, a derivative thereof, a metabolite thereof, or a biosynthetic intermediate thereof, or a biosynthetic intermediate thereof, and an altered content of a phytosterol, or a derivative thereof, a metabolite thereof, or a biosynthetic intermediate thereof, a cholesterol, or a derivative thereof, a metabolite thereof, or a biosynthetic intermediate thereof; a phytocholesterol, or a derivative thereof, a metabolite thereof, or a biosynthetic intermediate thereof, a cholestenol, or a derivative thereof, a metabolite thereof, or a biosynthetic intermediate thereof, or a phytocholestenol, or a derivative thereof, a metabolite thereof, or a biosynthetic intermediate thereof.

In some embodiments, a method of reducing the content of at least one steroidal alkaloid, a derivative thereof, a metabolite thereof, or a biosynthetic intermediate thereof, at least one steroidal saponin, a derivative thereof, a metabolite thereof, or a biosynthetic intermediate thereof, or at least one triterpenoid saponin, a derivative thereof, a metabolite thereof, or a biosynthetic intermediate thereof, comprises genetically modifying at least one plant cell or at least one cell of a plant or plant part, said genetically modified plant cell comprising a reduced content of at least one steroidal alkaloid, a derivative thereof, a metabolite thereof, at least one steroidal saponin, a derivative thereof, a metabolite thereof, or a biosynthetic intermediate thereof, or at least one triterpenoid saponin, a derivative thereof, a metabolite thereof, or a biosynthetic intermediate thereof, or a biosynthetic intermediate thereof, and an increased content of a phytosterol, or a derivative thereof, a metabolite thereof, or a biosynthetic intermediate thereof, a cholesterol, or a derivative thereof, a metabolite thereof, or a biosynthetic intermediate thereof; a phytocholesterol, or a derivative thereof, a metabolite thereof, or a biosynthetic intermediate thereof, a cholestenol, or a derivative thereof, a metabolite thereof, or a biosynthetic intermediate thereof, or a phytocholestenol, or a derivative thereof, a metabolite thereof, or a biosynthetic intermediate thereof.

In some embodiments, a method of increasing the content of at least one steroidal alkaloid, a derivative thereof, a metabolite thereof, or a biosynthetic intermediate thereof, at least one steroidal saponin, a derivative thereof, a metabolite thereof, or a biosynthetic intermediate thereof, or at least one triterpenoid saponin, a derivative thereof, a metabolite thereof, or a biosynthetic intermediate thereof, comprises genetically modifying at least one plant cell or at least one cell of a plant or plant part, said genetically modified plant cell comprising an increased content of at least one steroidal alkaloid, a derivative thereof, a metabolite thereof, or a biosynthetic intermediate thereof, at least one steroidal saponin, a derivative thereof, a metabolite thereof, or a biosynthetic intermediate thereof, or at least one triterpenoid saponin, a derivative thereof, a metabolite thereof, or a biosynthetic intermediate thereof, and an altered content of a phytosterol, or a derivative thereof, a metabolite thereof, or a biosynthetic intermediate thereof, a cholesterol, or a derivative thereof, a metabolite thereof, or a biosynthetic intermediate thereof; a phytocholesterol, or a derivative thereof, a metabolite thereof, or a biosynthetic intermediate thereof, a cholestenol, or a derivative thereof, a metabolite thereof, or a biosynthetic intermediate thereof; or a phytocholestenol, or a derivative thereof, a metabolite thereof, or a biosynthetic intermediate thereof.

In some embodiments, a method of increasing the content of at least one steroidal alkaloid, a derivative thereof, a metabolite thereof, or a biosynthetic intermediate thereof, at least one steroidal saponin, a derivative thereof, a metabolite thereof, or a biosynthetic intermediate thereof, or at least one triterpenoid saponin, a derivative thereof, a metabolite thereof, or a biosynthetic intermediate thereof, comprises genetically modifying at least one plant cell or at least one cell of a plant or plant part, said genetically modified plant cell comprising an increased content of at least one steroidal alkaloid, a derivative thereof, a metabolite thereof, or a biosynthetic intermediate thereof, at least one steroidal saponin, a derivative thereof, a metabolite thereof, or a biosynthetic intermediate thereof, or at least one triterpenoid saponin, a derivative thereof, a metabolite thereof, or a biosynthetic intermediate thereof, and a reduced content of a phytosterol, or a derivative thereof, a metabolite thereof, or a biosynthetic intermediate thereof, a cholesterol, or a derivative thereof, a metabolite thereof, or a biosynthetic intermediate thereof; a phytocholesterol, or a derivative thereof, a metabolite thereof, or a biosynthetic intermediate thereof, a cholestenol, or a derivative thereof, a metabolite thereof, or a biosynthetic intermediate thereof; or a phytocholestenol, or a derivative thereof, a metabolite thereof, or a biosynthetic intermediate thereof.

In some embodiments, the genetically modified cell is a plant cell. Plant cells can be obtained from a wide range of plants, as discussed below. The plant cell may be a leaf cell, a petiole cell, a plant stem or stalk cell, a root cell, a bud cell, a tuber cell, a bean cell, a grain or kernel cell, a fruit cell, a nut cell, a legume cell, a seed or seed cell, a bract cell, a callus cell, and a flower cell. In some embodiments, a genetically modified cell may then be comprised in a plant leaf, in a plant petiole, in a plant stem or stalk, in a plant root, in a plant bud, in a plant tuber, in a plant bean, in a plant grain or kernel, in a plant fruit, in a plant nut, in a plant legume, in a plant seed, in a plant bract, in a plant callus, or in a plant flower.

In some embodiments, the genetically modified plant cell is grown as such in culture, independent of a plant or plant part. Methods for culturing plants and plant parts are well known in the art, for example see Ochoa-Villarreal M, Howat S, Hong S, et al. Plant cell culture strategies for the production of natural products. BMB Rep. 2016; 49(3):149-158, which is incorporated herein in its entirety.

In some embodiments, the genetically modified plant cell is grown as such in culture, independent of a plant or plant part and is utilized in a method provided herein to produce steroidal alkaloids, steroidal saponins, or triterpenoid saponins. Suspensions of genetically modified cells and tissue cultures derived from the genetically modified cells are also encompassed within the scope described herein. The cell suspension and tissue cultures can be used for the production of a desired steroidal alkaloid, a derivative thereof, a metabolite thereof, or a biosynthetic intermediate thereof, a desired steroidal saponin, a derivative thereof, a metabolite thereof, or a biosynthetic intermediate thereof, a desired triterpenoid saponin, a derivative thereof, a metabolite thereof, or a biosynthetic intermediate thereof and, which are then extracted from the cells or the growth medium. Alternatively, the genetically modified plant cell and/or tissue culture are used for regenerating a transgenic plant having modified expression or overexpression of a saponin beta-amyrin synthase, a cytochrome P450, a glycosyltransferase, an acyltransferase, a glucuronosyltransferase, or a CSLG, therefore expressing or overexpressing a saponin beta-amyrin synthase, a cytochrome P450, a glycosyltransferase, an acyltransferase, a UDP-glucose 6-dehydrogenase 1, and a cellulose synthase like G (CSLG) in a cell or in a plant, as compared to a corresponding unmodified cell or plant, respectively. Therefore, having modified content of a steroidal alkaloid, a derivative thereof, a metabolite thereof, or a biosynthetic intermediate thereof, a steroidal saponin, a derivative thereof, a metabolite thereof, or a biosynthetic intermediate thereof, or a triterpenoid saponin, a derivative thereof, a metabolite thereof, or a biosynthetic intermediate thereof.

In some embodiments, the genetically modified plant cell is comprised in a plant. In some embodiments, the genetically modified plant cell is comprised in a plant, wherein the plant is utilized in a method provided herein to produce steroidal alkaloids, steroidal saponins, or triterpenoid saponins. In some embodiments, the genetically modified plant cell is comprised in a plant tissue. In some embodiments, the genetically modified plant cell is comprised in a plant tissue, wherein the plant tissue is utilized in a method provided herein to produce steroidal alkaloids, steroidal saponins, or triterpenoid saponins. In some embodiments, the genetically modified plant cell is comprised in a plant organ. In some embodiments, the genetically modified plant cell is comprised in a plant organ, wherein the plant organ is utilized in a method provided herein to produce steroidal alkaloids, steroidal saponins, or triterpenoid saponins. In some embodiments, the genetically modified plant cell is comprised in a plant part. In some embodiments, the genetically modified plant cell is comprised in a plant part, wherein the plant part is utilized in a method provided herein to produce steroidal alkaloids, steroidal saponins, or triterpenoid saponins.

In some embodiments, a genetically modified cell comprises a plant cell comprising a leaf cell, a petiole cell, a plant stem or stalk cell, a root cell, a bud cell, a tuber cell, a bean cell, a grain or kernel cell, a fruit cell, a nut cell, a legume cell, a seed or seed cell, a bract cell, a callus cell, or a flower cell. In some embodiments, a genetically modified cell comprises a plant cell comprised in a plant leaf, in a plant petiole, in a plant stem or stalk, in a plant root, in a plant bud, in a plant tuber, in a plant bean, in a plant grain or kernel, in a plant fruit, in a plant nut, in a plant legume, in a plant seed, in a plant bract, in a plant callus, or in a plant flower, or in a combination thereof. In some embodiments, a genetically modified plant cell is comprised within a plant part, wherein said plant part comprises a leaf, a petiole, a plant stem or stalk, a root, a bud, a tuber, a bean, a grain or kernel, a fruit, a nut, a legume, a seed or seed, a bract, a callus, or a flower.

In some embodiments, a genetically modified plant cell may be utilized in a method of producing a steroidal alkaloid, a steroidal saponin, or a triterpenoid saponin as described herein.

In some embodiments, a genetically modified cell comprises a genetically modified plant cell (e.g., cultured plant cells). In other embodiments, a genetically modified plant cell is comprised in plant or a plant organ(s), plant tissue(s), or plant part(s). In some embodiments, the term “plant” may be used interchangeably with the term “plant part” having all the same meanings and qualities.

In some embodiments, a genetically modified plant cell, plant, plant organ, plant tissue, or plant part may be utilized in a method of producing a steroidal alkaloid, a steroidal saponin, or a triterpenoid saponin as described herein.

The content of a steroidal alkaloid, a steroidal saponin, or a triterpenoid saponin, a derivative thereof, a metabolite thereof, or a biosynthetic intermediate thereof present in a genetically modified cell or plant or plant part is measured as exemplified hereinbelow and as is known to a person skilled in the art.

In some embodiments, an offspring plant comprises increased contents of a steroidal alkaloid, a derivative thereof, a metabolite thereof, or a biosynthetic intermediate thereof, a steroidal saponin, a derivative thereof, a metabolite thereof, or a biosynthetic intermediate thereof, a triterpenoid saponin, a derivative thereof, a metabolite thereof, or a biosynthetic intermediate thereof, compared to at least one of the progenitor plants.

In some embodiments, a plant or plant cell or plant tissue or plant part as disclosed herein is selected from the Caryophyllales order, the Solanales order, the Fabales order, the Malvales order, the Apiales order, the Brassicales order, the Poales order, the Lamiales order, the Cucurbitales order, the Asterales order, the Malpighiales order, the Rosales order, the Fagales order, the Sapindales order, the Arecales order, the Ericales order, the Gentianales order, the Ranunculales order, the Zingiberales order, the Saxifragales order, the Vitales order, the Pinales order, the Cornales order, the Asparagales order, the Dioscoreales order, and the Liliales order. In some embodiments, a plant or plant cell or plant tissue or plant part as disclosed herein comprises a cell from a plant selected from the Caryophyllales order, the Solanales order, the Fabales order, the Malvales order, the Apiales order, the Brassicales order, the Poales order, the Lamiales order, the Cucurbitales order, the Asterales order, the Malpighiales order, the Rosales order, the Fagales order, the Sapindales order, the Arecales order, the Ericales order, the Gentianales order, the Ranunculales order, the Zingiberales order, the Saxifragales order, the Vitales order, the Pinales order, the Cornales order, the Asparagales order, the Dioscoreales order, and the Liliales order.

In some embodiments, a plant or plant cell or plant tissue or plant part as disclosed herein comprises a cell from a plant in the Poales order, the Caryophyllales order, the Solanales order, the Fabales order, the Malvales order, the Apiales order, the Brassicales order, the Asparagales order, the Dioscoreales order, or the Liliales order.

In some embodiments, a plant or plant cell or plant tissue or plant part as disclosed herein is in the Caryophyllales order and is selected from the group of genera consisting of the Spinacia genus (e.g., Spinacia oleracea), the Chenopodium genus (e.g., Chenopodium quinoa), the Beta genus (e.g., Beta vulgaris) the Rheum genus (e.g., Rheum hybridum, Rheum rhaponticum, Rheum rhabarbarum, Rheum ribes), the Vaccaria genus (e.g., Vaccaria hispanica), the Saponaria genus (e.g., Saponaria vaccaria), and the Gypsophila genus (e.g., Gypsophila paniculata). In some embodiments, a Caryophyllales plant is selected from the group consisting of spinach, beetroot, and quinoa.

In some embodiments, a plant or plant cell or plant tissue or plant part as disclosed herein is in the Solanales order and is selected from the group of genera consisting of the Nicotiana genus (e.g., Nicotiana benthamiana), the Solanum genus (e.g., Solanum lycopersicum, Solanum tuberosum, Solanum melongena, Solanum pennellii, Solanum chacoense, Solanum dulcamara), the Capsicum genus (e.g., Capsicum annuum), the Hyoscyamus genus, the Datura genus, and the Atropa genus. In some embodiments, a plant as disclosed herein comprises a Solanaceae crop plant. In some embodiments, a Solanaceae crop plant is selected from the group consisting of Solanum lycopersicum, Solanum pennellii, Solanum tuberosum, Solanum chacoense, Capsicum annuum, Solanum dulcamara, and Solanum melongena. In some embodiments, a Solanaceae or Solanales plant is selected from the group consisting of ground cherry, eggplant, potato, tomato, wild tomato, potato, wild potato, pepper, bell pepper, cayenne pepper, chili pepper, pimiento, tabasco pepper, tobacco, and bittersweet.

In some embodiments, the plant is in the Fabales order and is selected from the group of genera consisting of the Glycyrrhiza genus (e.g., Glycyrrhiza uralensis, Glycyrrhiza glabra [licorices]), the Medicago genus (e.g., Medicago sativa, Medicago truncatula), the Quillaja genus (e.g., Quillaja saponaria), the Glycine genus (e.g., Glycine max [soy/soybean]), the Lotus genus (e.g., Lotus japonicus), the Cicer genus (e.g., Cicer arietinum [chickpea, garbanzo bean]), the Phaseolus genus (e.g., Phaseolus vulgaris [string bean, common bean, French bean]), the Pisum genus (e.g., Pisum sativum [pea]), the Arachis genus (e.g., Arachis hypogaea [peanut]), the Lupinus genus (e.g., Lupinus albus [lupin/lupine]), and the Acacia genus. In some embodiments, a Fabales plant is selected from the group consisting of pea, alfalfa, soy, Lotus japonicus, and licorice.

In some embodiments, the plant is from the Malvales order and is selected from the Theobroma genus (e.g., Theobroma cacao).

In some embodiments, the plant is from the Apiales order and is selected from the group of genera consisting of the Daucus genus (e.g., Daucus carota), the Apium genus (e.g., Apium graveolens), the Petroselinum genus (e.g., Petroselinum crispum), the Panax genus (e.g., Panax ginseng), the Bupleurum genus, the Hedera genus, and the Centella genus (e.g., Centella asiatica).

In some embodiments, the plant is from the Brassicales order and is selected from the group of genera consisting of the Arabidopsis genus (e.g., Arabidopsis thaliana), the Brassica genus (e.g., Brassica oleracea [cabbages], Brassica juncea [white mustard], Brassica nigra [black mustard], Brassica napus), the Capparis genus (e.g., Capparis spinosa [caper]), and the Carica genus (e.g., Carica papaya [papaya]).

In some embodiments, the plant is from the Poales order and is selected from the group of genera consisting of the Oryza genus (e.g., Oryza sativa and Oryza glaberrima [rice]), the Hordeum genus (e.g., Hordeum vulgare [barley]), the Avena genus (e.g., Avena sativa [oat], Avena strigosa), and the Triticum genus (e.g., Triticum spelta [spelt]).

In some embodiments, the plant is from the Lamiales order and is selected from the group of genera consisting of the Salvia genus (e.g., Salvia hispanica [chia]), the Sesamum genus (e.g., Sesamum indicum [sesame, benne]), and the Olea genus (e.g., Olea europaea [olive]).

In some embodiments, the plant is from the Cucurbitales order and is selected from the Cucurbita genus (e.g., squash/pumpkin, including, but not limited to, Cucurbita pepo, Cucurbita maxima, Cucurbita argyrosperma, or Cucurbita moschata).

In some embodiments, the plant is from the Asterales order and is selected from the group of genera consisting of the Helianthus genus (e.g., Helianthus annuus [sunflower], Helianthus verticallatus [whorled sunflower], Helianthus tuberosus [Jerusalem artichoke]), the Artemesia genus (e.g., Artemesia annua), the Galatella (Aster) genus (e.g., Galatella sedifolia), and the Taraxacum genus (e.g., Taraxacum officinale [dandelion]).

In some embodiments, the plant is from the Malpighiales order and is selected from the group of genera consisting of the Linum genus (e.g., Linum usitatissimum [flax, linseed]), the Bruguiera genus (e.g., Bruguiera gymnorhiza), the Euphorbia genus (e.g., Euphorbia tirucalli), the Ricinus genus (e.g., Ricinus communis [castor]), the Kandelia genus (e.g., Kandelia candel), and the Rhizophora genus (e.g., Rhizophora stylosa).

In some embodiments, the plant is from the Rosales order and is selected from the group of genera consisting of the Prunus genus (e.g., Prunus dulcis [almond], Prunus amygdalus) and the Cannabis genus (e.g., hemp, including Cannabis sativa).

In some embodiments, the plant is from the Fagales order and is selected from the group of genera consisting of the Corylus genus (e.g., hazel/hazelnut/cobnut/filbert nut, including, but not limited to, Corylus avellana), the Betula genus (e.g., Betula pendula [silver birch], Betula pubescens [white or downy birch], Betula platyphylla) and the Juglans genus (e.g., Juglans regia [Persian or English walnut], Juglans nigra [black walnut], Juglans cinera [butternut]).

In some embodiments, the plant is from the Sapindales order and is selected from the group of genera consisting of the Anacardium genus (e.g., Anacardium occidentale [cashew]), the Pistacia genus (e.g., Pistacia vera [pistachio]), the Citrus genus (numerous species and hybrids), the Aesculus genus (e.g., Aesculus hippocastanum, Aesculus turbinata), and the Peganum genus.

In some embodiments, the plant is from the Arecales order and is selected from the Cocus genus (e.g., Cocus nucifera [coconut]).

In some embodiments, the plant is from the Ericales order and is selected from the Maesa genus.

In some embodiments, the plant is from the Gentianales order and is selected from the group of genera consisting the Nerium genus (e.g., Nerium oleander), the Gentianamacrophylla genus (e.g., Gentianamacrophylla straminea), the Catharanthus genus (e.g., Catharanthus roseus), the Rauwolfia genus, and the Cinchona genus.

In some embodiments, the plant is from the Ranunculales order and is selected from the genera consisting of the Nigella genus (e.g., Nigella sativa), the Papaver genus, the Eschscholtzia genus (e.g., Eschscholtzia californica), the Coptis genus, the Berberis genus, and the Thalictrum genus.

In some embodiments, the plant is from the Zingiberales order and is selected from the Cheilocostus genus (e.g., Cheilocostus speciosus).

In some embodiments, the plant is from the Saxifragales order and is selected from the Kalanchoe genus (e.g., Kalanchoe daigremontiana).

In some embodiments, the plant is from the Vitales order and is selected from the Vitis genus (e.g., Vitis vinfera [grape]).

In some embodiments, the plant is from the Pinales order and is selected from the Taxus genus (e.g., Taxus brevifolia, Taxus baccdata, Taxus cuspidata, Taxus canadensis, Taxus floridana).

In some embodiments, the plant is from the Cornales order and is selected from the Camptotheca genus (e.g., Camptotheca acuminata). In some embodiments, the plant is from the Asparagales order and is selected from the Agave genus (e.g., Agave americana, Agave attenuata, Agave tequilana), the Asparagus genus (e.g., Asparagus officinalis), or the Yucca genus (e.g., Yucca flamentosa). In some embodiments, the plant is from the Dioscoreales order and is selected from the Borderea genus, the Dioscorea genus, the Epipetrum genus, the Rajania genus, the Stenomeris genus, or the Tamus genus. In some embodiments, the plant is from the Liliales order and is selected from the Liliaceae family (e.g., Clintonia borealis, Nomocharis aperta, Calochortus catalinae, Streptopus lariceolatus).

A skilled artisan would appreciate that plant breeding can be accomplished through many different techniques ranging from simply selecting plants with desirable characteristics for propagation, to methods that make use of knowledge of genetics and chromosomes, to more complex molecular techniques.

A skilled artisan would appreciate that the term “hybrid plant” may encompass a plant generated by crossing two plants of interest, propagating by seed or tissue and then growing the plants. When plants are crossed sexually, the step of pollination may include cross pollination or self-pollination or back crossing with an untransformed plant or another transformed plant. Hybrid plants include first generation and later generation plants.

### Biological Activity

In some embodiments, the genetically modified cell or genetically modified plant comprises an endogenous enzyme comprising the same or similar activity with an enzyme encoded by the at least one heterologous gene. In other embodiments, the genetically modified cell or genetically modified plant does not comprise an endogenous enzyme comprising the same or similar activity with an enzyme encoded by the at least one heterologous gene. In some embodiments, the genetically modified plant cell or plant comprises an endogenous enzyme comprising altered activity of a steroidal alkaloid, a steroidal saponin, or a triterpenoid saponin biosynthesis enzyme.

In some embodiments, the biological activity of at least one saponin beta-amyrin synthase, cytochrome P450, a glycosyltransferase, acyltransferase, a glucuronosyltransferase, or CSLG, or combination thereof encoded by the heterologous gene is altered compared with an endogenous saponin beta-amyrin synthase, cytochrome P450, glycosyltransferase, acyltransferase, glucuronosyltransferase, or CSLG protein present in the genetically modified cell or genetically modified plant. In some embodiments, the biological activity of at least one endogenous saponin beta-amyrin synthase, cytochrome P450, a glycosyltransferase, acyltransferase, a glucuronosyltransferase, or CSLG, or combination thereof is altered compared with the endogenous saponin beta-amyrin synthase, cytochrome P450, glycosyltransferase, acyltransferase, glucuronosyltransferase, or CSLG protein present in the non-genetically modified cell or non-genetically modified plant.

A skilled artisan would recognize that the term “biological activity” refers to any activity associated with a protein that can be measured by an assay. In some embodiments, the biological activity of a saponin beta-amyrin synthase, a cytochrome P450, a glycosyltransferase, an acyltransferase, a glucuronosyltransferase, or a CSLG comprises an enzyme activity necessary for the biosynthesis of a steroidal alkaloid, a derivative thereof, a metabolite thereof, or a biosynthetic intermediate thereof, a steroidal saponin, a derivative thereof, a metabolite thereof, or a biosynthetic intermediate thereof, or a triterpenoid saponin, a derivative thereof, a metabolite thereof, or a biosynthetic intermediate thereof in a cell or a plant. In some embodiments, the biological activity of a saponin beta-amyrin synthase, a cytochrome P450, a glycosyltransferase, an acyltransferase, a glucuronosyltransferase, or a CSLG affects the content of a steroidal alkaloid, a derivative thereof, a metabolite thereof, or a biosynthetic intermediate thereof, a steroidal saponin, a derivative thereof, a metabolite thereof, or a biosynthetic intermediate thereof, or a triterpenoid saponin, a derivative thereof, a metabolite thereof, or a biosynthetic intermediate thereof, in at least a part of a plant. In some embodiments, an altered biological activity comprises increased enzyme activity. In some embodiments, an altered biological activity comprises decreased enzyme activity. In some embodiments, an altered biological activity comprises increased stability of the polypeptide. In some embodiments, an altered biological activity comprises decreased stability of the polypeptide. In some embodiments, an altered biological activity comprises increased expression so higher levels of the enzyme polypeptide. In some embodiments, an altered biological activity comprises decreased expression of the enzyme polypeptide.

In some embodiments, the biological activity of an enzyme is altered compared with a control enzyme.

In some embodiments, the altered biological activity comprises (a) increased enzyme activity of the saponin beta-amyrin synthase, cytochrome P450, glycosyltransferase, acyltransferase, glucuronosyltransferase, or CSLG; or increased stability of the saponin beta-amyrin synthase, cytochrome P450, glycosyltransferase, acyltransferase, glucuronosyltransferase, or CSLG; or decreased enzyme activity of the saponin beta-amyrin synthase, cytochrome P450, glycosyltransferase, acyltransferase, glucuronosyltransferase, or CSLG; or decreased stability of the saponin beta-amyrin synthase, cytochrome P450, glycosyltransferase, acyltransferase, glucuronosyltransferase, or CSLG.

In some embodiments, the biological activity of a saponin beta-amyrin synthase, cytochrome P450, glycosyltransferase, acyltransferase, glucuronosyltransferase, or CSLG is increased. In some embodiments, the biological activity of a saponin beta-amyrin synthase, cytochrome P450, glycosyltransferase, acyltransferase, glucuronosyltransferase, or CSLG is decreased. In some embodiments, a saponin beta-amyrin synthase, cytochrome P450, glycosyltransferase, acyltransferase, glucuronosyltransferase, or CSLG has increased stability. In some embodiments, a saponin beta-amyrin synthase, cytochrome P450, glycosyltransferase, acyltransferase, glucuronosyltransferase, or CSLG has decreased stability.

In some embodiments, the altered biological activity comprises increased enzyme activity of said at least one saponin beta-amyrin synthase, cytochrome P450, glycosyltransferase, acyltransferase, glucuronosyltransferase, or CSLG; or increased stability of said at least one saponin beta-amyrin synthase, cytochrome P450, glycosyltransferase, acyltransferase, glucuronosyltransferase, or CSLG; or decreased enzyme activity of said at least one saponin beta-amyrin synthase, cytochrome P450, glycosyltransferase, acyltransferase, glucuronosyltransferase, or CSLG; or decreased stability of said at least one saponin beta-amyrin synthase, cytochrome P450, glycosyltransferase, acyltransferase, glucuronosyltransferase, or CSLG; compared to the biological activity in the endogenous enzyme, if present, in said genetically modified cell or genetically modified plant.

### Transgenic Plants, Transgenic Yeast, Transgenic Algae, Transgenic Insect Cells, Transgenic Bacterium

Cloning of a polynucleotide encoding an enzyme encoded by the at least one heterologous gene, wherein the enzyme comprises at least one saponin beta-amyrin synthase, cytochrome P450, glycosyltransferase, acyltransferase, glucuronosyltransferase, or CSLG can be performed by any method as is known to a person skilled in the art. Cloning of a polynucleotide encoding an at least one saponin beta-amyrin synthase, cytochrome P450, glycosyltransferase, acyltransferase, glucuronosyltransferase, or CSLG as described herein, can be performed by any method as is known to a person skilled in the art. In certain embodiments, a polynucleotide encoding a CSLG enzyme is cloned. Various DNA constructs may be used to express the desired gene in a desired cell or organism. In some embodiments, various DNA constructs are used to expression a CSLG gene in a cell. In some embodiments, various DNA constructs are used to expression a CSLG gene in a plant cell, a yeast cell, an insect cell, an algal cell, or a bacterium. In some embodiments, various DNA constructs are used to expression a CSLG gene in a plant cell. In some embodiments, various DNA constructs are used to expression a CSLG gene in a yeast cell. In some embodiments, various DNA constructs are used to expression a CSLG gene in a plant cell or a yeast cell. In some embodiments, various DNA constructs are used to expression a CSLG gene in an algal cell. In some embodiments, various DNA constructs are used to expression a CSLG gene in an insect cell. In some embodiments, various DNA constructs are used to expression a CSLG gene in a bacterium. In some embodiments, various DNA constructs are used to expression a heterologous CSLG gene in the cell. In some embodiments, when the cell is a plant cell the CSLG gene is a heterologous gene. In some embodiments, when the cell is a plant cell the CSLG gene is a homologous gene, wherein said expression is altered, for example increased or decreased, or said CSLG gene is mutated to alter the activity of the CSLG enzyme. In some embodiments, when the cell is a plant cell, the plant cell is comprised within a plant or a plant part.

According to certain embodiments, the gene may comprise part of an expression vector comprising all necessary elements for expression of the gene and optional regulatory components.

According to certain embodiments, the expression is controlled by a constitutive promoter. According to other embodiments, the expression is controlled by a transient promoter. According to certain embodiments, the constitutive promoter is specific to a cell or to a plant or plant tissue. According to some embodiments, the tissue specific promoter is selected from the group consisting of root, tuber, leaves and fruit specific promoter. Root specific promoters are described, e.g. in Martinez, E. et al. 2003. Curr. Biol. 13:1435-1441. Fruit specific promoters are described among others in Estornell L. H et al. 2009. Plant Biotechnol. J. 7:298-309 and Fernandez A. I. Et al. 2009 Plant Physiol. 151:1729-1740. Tuber specific promoters are described, e.g. in Rocha-Sosa M, et al., 1989. EMBO J. 8:23-29; McKibbin R. S. et al., 2006. Plant Biotechnol J. 4(4):409-18. Leaf specific promoters are described, e.g. in Yutao Yang, Guodong Yang, Shijuan Liu, Xingqi Guo and Chengchao Zheng. Science in China Series C: Life Sciences. 46: 651-660. Accordingly, in some embodiments, a promoter comprises a yeast specific promoter. Accordingly, in some embodiments, a promoter comprises an algal specific promoter.

According to certain embodiments, the expression vector further comprises regulatory elements at the 3′ non-coding sequence. As used herein, the “3′ non-coding sequences” refer to DNA sequences located downstream of a coding sequence and include polyadenylation recognition sequences and other sequences encoding regulatory signals capable of affecting mRNA processing or gene expression. The polyadenylation signal is usually characterized by affecting the addition of polyadenylic acid tracts to the 3′ end of the mRNA precursor. For plants, the use of different 3′ non-coding sequences is exemplified by Ingelbrecht I L et al. (1989. Plant Cell 1:671-680).

Those skilled in the art will appreciate that the various components of the nucleic acid sequences and the transformation vectors described herein are operatively linked, so as to result in expression of said nucleic acid or nucleic acid fragment. Techniques for operatively linking the components of the constructs and vectors described herein are well known to those skilled in the art. Such techniques include the use of linkers, such as synthetic linkers, for example, including one or more restriction enzyme sites.

One skilled in the art would appreciate that the term “operably linked” may encompass the association of nucleic acid sequences on a single nucleic acid fragment so that the function of one is regulated by the other. For example, a promoter is operably linked with a coding sequence when it is capable of regulating the expression of that coding sequence (i.e., that the coding sequence is under the transcriptional control of the promoter). Coding sequences can be operably linked to regulatory sequences in a sense or antisense orientation.

Methods for transforming a plant according to the teachings disclosed herein are known to those skilled in the art. As used herein the term “transformation” or “transforming” describes a process by which a foreign DNA, such as a DNA construct, including expression vector, enters and changes a recipient cell into a transformed, genetically altered or transgenic cell. Transformation may be stable, wherein the nucleic acid sequence is integrated into the organism genome and as such represents a stable and inherited trait, or transient, wherein the nucleic acid sequence is expressed by the cell transformed but is not integrated into the genome, and as such represents a transient trait. According to some embodiments the nucleic acid sequence disclosed herein is stably transformed into the cell.

The genetically altered cells or plants having altered content of the desired at least one saponin beta-amyrin synthase, cytochrome P450, glycosyltransferase, acyltransferase, glucuronosyltransferase, or CSLG; and altered content of at least one steroidal alkaloid, derivative thereof, metabolite thereof, or biosynthetic intermediate thereof, at least one steroidal saponin, derivative thereof, metabolite thereof, or biosynthetic intermediate thereof; or at least one triterpenoid saponin, derivative thereof, metabolite thereof, or biosynthetic intermediate thereof. In some embodiments, genetically modified cells or plants produced as described herein are typically first selected based on the expression of the gene or protein. Cells or plants having expressing the enzyme encoding from the at least one heterologous gene may be identified and, are then analyzed for the content of at least one saponin beta-amyrin synthase, cytochrome P450, glycosyltransferase, acyltransferase, glucuronosyltransferase, or CSLG and/or at least one steroidal alkaloid, derivative thereof, metabolite thereof, or biosynthetic intermediate thereof, at least one steroidal saponin, derivative thereof, metabolite thereof, or biosynthetic intermediate thereof; or a triterpenoid saponin, a derivative thereof, a metabolite thereof, or a biosynthetic intermediate thereof.

In some embodiments, the genetically altered plants having altered content of the desired at least one steroidal alkaloid, derivative thereof, metabolite thereof, or biosynthetic intermediate thereof, at least one steroidal saponin, derivative thereof, metabolite thereof, or biosynthetic intermediate thereof, or at least one triterpenoid saponin, derivative thereof, metabolite thereof, or biosynthetic intermediate thereof, according to the teachings herein are first selected based on the expression of the gene or protein. Plants having enhanced or aberrant expression of the gene or protein, are then analyzed for the content of at least one steroidal alkaloid, derivative thereof, metabolite thereof, or biosynthetic intermediate thereof, at least one steroidal saponin, derivative thereof, metabolite thereof, or biosynthetic intermediate thereof, or at least one triterpenoid saponin, derivative thereof, metabolite thereof, or biosynthetic intermediate thereof. In some embodiments, derivatives of steroidal alkaloids, steroidal saponins, or triterpenoid saponins comprise glycosylated derivatives of, respectively, steroidal alkaloids, steroidal saponins, or triterpenoid saponins.

Detection of at least one saponin beta-amyrin synthase, cytochrome P450, glycosyltransferase, acyltransferase, glucuronosyltransferase, or CSLG gene and/or at least one steroidal alkaloid, derivative thereof, metabolite thereof, or biosynthetic intermediate thereof; at least one steroidal saponin, derivative thereof, metabolite thereof, or biosynthetic intermediate thereof; or a triterpenoid saponin, a derivative thereof, a metabolite thereof, or a biosynthetic intermediate thereof is performed employing standard methods of molecular genetics, known to a person of ordinary skill in the art. Similarly, purification and/or detection of at least one steroidal alkaloid, derivative thereof, metabolite thereof, or biosynthetic intermediate thereof; at least one steroidal saponin, derivative thereof, metabolite thereof, or biosynthetic intermediate thereof, or a triterpenoid saponin, a derivative thereof, a metabolite thereof, or a biosynthetic intermediate thereof is performed employing standard methods of molecular genetics or protein chemistry known to a person of ordinary skill in the art.

For measuring the gene(s), cDNA or mRNA should be obtained from a cell or a plant in which the nucleic acid is expressed. The sample may be further processed before the detecting step. For example, the polynucleotides in the cell or tissue sample may be separated from other components of the sample, may be amplified, etc. All samples obtained from an organism, including those subjected to any sort of further processing are considered to be obtained from the organism.

For measuring the gene(s) or silencing molecule(s) expression, cDNA or mRNA should be obtained from an organ in which the nucleic acid is expressed. The sample may be further processed before the detecting step. For example, the polynucleotides in the cell or tissue sample may be separated from other components of the sample, may be amplified, etc. All samples obtained from an organism, including those subjected to any sort of further processing are considered to be obtained from the organism.

Detection of the gene(s) in some embodiments, requires amplification of the polynucleotides taken from the candidate genetically modified cell or genetically modified plant or part thereof. In some embodiments a plant part comprises a plant organ or a plant tissue.

Methods for DNA amplification are known to a person skilled in the art. Most commonly used method for DNA amplification is PCR (polymerase chain reaction; see, for example, PCR Basics: from background to Bench, Springer Verlag, 2000; Eckert et al., 1991. PCR Methods and Applications 1:17). Additional suitable amplification methods include the ligase chain reaction (LCR), transcription amplification and self-sustained sequence replication, and nucleic acid-based sequence amplification (NASBA).

According to certain embodiments, the nucleic acid sequence comprising the at least one saponin beta-amyrin synthase, cytochrome P450, glycosyltransferase, acyltransferase, glucuronosyltransferase, or CSLG, or any combination thereof may in some embodiments, further comprises a nucleic acid sequence encoding a selectable marker. According to certain embodiments, the selectable marker confers resistance to antibiotic or to an herbicide; in these embodiments the transgenic cells or plants are selected according to their resistance to the antibiotic or herbicide.

### Breeding

In some embodiments, transformation techniques including breeding through transgene editing, use of transgenes, use of transient expression of a gene or genes, or use of molecular markers, or any combination thereof, may be used in the breeding of a plant having an altered expression. If transformation techniques require use of tissue culture, transformed cells may be regenerated into plants in accordance with techniques well known to those of skill in the art. The regenerated plants may then be grown and crossed with the same or different plant varieties using traditional breeding techniques to produce seed, which are then selected under the appropriate conditions.

The content of steroidal alkaloids and/or steroidal saponins is measured as exemplified hereinbelow and as is known to a person skilled in the art.

In some embodiments, an offspring plant comprises decreased anti-nutritional contents or decreased toxins compared to at least one of the progenitor plants. In some embodiments, an offspring plant comprises improved resistance to a plant pathogen, pest, or predator compared to at least one of the progenitor plants.

In some embodiments, a plant as disclosed herein comprises a crop plant from any of the orders listed in the previous section. In some embodiments, a plant as disclosed herein comprises a crop plant in the Poales order, the Caryophyllales order, the Solanales order, the Fabales order, the Malvales order, the Apiales order, the Brassicales order, the Asparagales order, the Dioscoreales order, or the Liliales order.

In some embodiments, when the plant comprises a crop plant in the Poales order, said plant is selected from the group of genera consisting of the Oryza genus, the Hordeum genus, the Avena genus, and the Triticum genus.

In some embodiments, when a plant is a crop plant in the Caryophyllales order, said plant is selected from the group of genera consisting of the Spinacia genus, the Chenopodium genus, the Beta genus, the Rheum genus, the Vaccaria genus, the Saponaria genus, and the Gypsophila genus. In some embodiments, the Caryophyllales plant is selected from the group consisting of spinach, beetroot, and quinoa.

In some embodiments, when a plant is a crop plant in the Solanales order, said plant from the group of genera consisting of the Solanum genus, the Capsicum genus, the Nicotiana genus, the Hyoscyamus genus, the Datura genus, and the Atropa genus. In some embodiments, when a plant is a crop plant in the Solanales order, said plant is selected from the group consisting of Solanum lycopersicum, Solanum pennellii, Solanum tuberosum, Solanum chacoense, Capsicum annuum, Solanum melongena, Solanum dulcamara, and Nicotiana benthamiana. In some embodiments, the Solanales plant is selected from the group consisting of ground cherry, eggplant, potato, tomato, pepper, bell pepper, cayenne pepper, chili pepper, pimiento, tabasco pepper, tobacco, and bittersweet. In some embodiments, the Solanales plant comprises a Solanaceae plant.

In some embodiments, when a plant is a crop plant in the Fabales order, said plant is selected from the group of genera consisting of the Glycyrrhiza genus, the Medicago genus, the Glycine genus, the Lotus genus, the Cicer genus, the Phaseolus genus, the Pisum genus, the Arachis genus, the Lupinus genus, and the Acacia genus. In some embodiments, the Fabales plant is selected from the group consisting of pea, alfalfa, soy, Lotus japonicus, and licorice.

In some embodiments, when a plant is a crop plant in the Malvales order, said plant is selected from the Theobroma genus.

In some embodiments, when a plant is a crop plant in the Apiales order, said plant is selected from the group of genera consisting of the Daucus genus, the Apium genus, the Petroselinum genus, the Panax genus, the Bupleurum genus, the Hedera genus, and the Centella genus.

In some embodiments, when a plant is a crop plant in the Brassicales order, said plant is selected from the group of genera consisting of the Arabidopsis genus, the Brassica genus, the Capparis genus, and the Carica genus.

In some embodiments, the plant is a crop plant of any order, family, genus, or species disclosed herein.

A skilled artisan would appreciate that plant breeding can be accomplished through many different techniques ranging from simply selecting plants with desirable characteristics for propagation, to methods that make use of knowledge of genetics and chromosomes, to more complex molecular techniques.

A skilled artisan would appreciate that the term “hybrid plant” may encompass a plant generated by crossing two plants of interest, propagating by seed or tissue and then growing the plants. When plants are crossed sexually, the step of pollination may include cross pollination or self-pollination or back crossing with an untransformed plant or another transformed plant. Hybrid plants include first generation and later generation plants. Disclosed herein is a method to manipulate and improve a plant trait, for a non-limiting example—increasing plant resistance, decreasing anti-nutritional properties in a plant, decreasing toxins or bitter tasting compounds in a plant, increasing pharmaceutical properties in a plant, or any combination thereof.

### Biomarkers

A skilled artisan would appreciate that the term “biomarker” comprises any measurable substance in an organism whose presence is indicative of a biological state or a condition of interest. In some embodiments, the presence of a biomarker is indicative of the presence of a compound or a group of compounds of interest. In some embodiments, the concentration of a biomarker is indicative of the concentration of a compound or a group of compounds of interest. In some embodiments, the concentration of a biomarker is indicative of an organism phenotype.

CSLG enzymes are hereby disclosed to have an essential role in the biosynthesis of steroidal alkaloids, steroidal saponins, and triterpenoid saponins found in many plants. Thus, in some embodiments, the expression level of CSLG is indicative of the capacity of a plant to produce steroidal alkaloids, or derivatives, metabolites, or biosynthetic intermediates thereof, steroidal saponins, or derivatives, metabolites, or biosynthetic intermediates thereof, triterpenoid saponins, or derivatives, metabolites, or biosynthetic intermediates thereof, or combinations thereof.

### Methods for Producing Steroidal Alkaloids, Steroidal Saponins, and Triterpenoid Saponins

Provided herein are methods of producing a steroidal alkaloid, a steroidal saponin, or a triterpenoid saponin in a genetically modified cell or plant comprising a genetically modified plant cell or a plant part comprising a genetically modified plant part, the methods comprising: introducing a polynucleotide sequence into said cell, wherein said polynucleotide sequence is optionally comprised in a vector, wherein said polynucleotide sequence comprises at least one heterologous gene encoding an enzyme, said enzyme selected from the group consisting of a saponin beta-amyrin synthase, a cytochrome P450, a glycosyltransferase, an acyltransferase, a UDP-glucose 6-dehydrogenase I, and a cellulose synthase like G (CSLG); and expressing said at least one heterologous gene in said cell. In some embodiments, the polynucleotide sequence introduced comprises multiple polynucleotide sequences, wherein each polynucleotide sequence comprises at least one heterologous gene encoding an enzyme, said enzyme selected from the group consisting of a saponin beta-amyrin synthase, a cytochrome P450, a glycosyltransferase, an acyltransferase, a UDP-glucose 6-dehydrogenase I, and a cellulose synthase like G (CSLG). In some embodiments, the polynucleotide sequence introduced comprises 2, 3, 4, 5, 6, 7, 8, 9, 10, or more polynucleotide sequences each polynucleotide sequence comprising at least one heterologous gene encoding an enzyme, said enzyme selected from the group consisting of a saponin beta-amyrin synthase, a cytochrome P450, a glycosyltransferase, an acyltransferase, a UDP-glucose 6-dehydrogenase I, and a cellulose synthase like G (CSLG). In some embodiments, the polynucleotide sequence introduced comprises 2, 3, 4, 5, 6, 7, 8, 9, 10, or more polynucleotide sequences each comprises only one heterologous gene encoding an enzyme, said enzyme selected from the group consisting of a saponin beta-amyrin synthase, a cytochrome P450, a glycosyltransferase, an acyltransferase, a UDP-glucose 6-dehydrogenase I, and a cellulose synthase like G (CSLG). In some embodiments, the polynucleotide sequence introduced comprises 2, 3, 4, 5, 6, 7, 8, 9, 10, or more polynucleotide sequences each comprising any combination of 1, 2, 3, 4, 5, 6, 7, 8, 9, or 10, or more heterologous genes encoding an enzyme, said enzyme selected from the group consisting of a saponin beta-amyrin synthase, a cytochrome P450, a glycosyltransferase, an acyltransferase, a UDP-glucose 6-dehydrogenase I, and a cellulose synthase like G (CSLG). A skilled artisan would appreciate that the polynucleotide sequence or sequences are introduced into a cell where they are expressed, resulting in expression of the encoded enzyme or enzymes in the genetically modified cell.

In some embodiments, disclosed herein is a method of producing a steroidal alkaloid, a steroidal saponin, or a triterpenoid saponin in a genetically modified cell, the method comprising:
(a) introducing an at least one heterologous gene into said cell, said at least one heterologous gene encoding a cellulose synthase like G (CSLG) enzyme, wherein said heterologous gene is optionally comprised in a vector; and
(b) expressing said at least one heterologous gene in said cell;
   wherein said cell comprises an increased content of at least one steroidal alkaloid, at least one steroidal saponin, or at least one triterpenoid saponin compared to a corresponding unmodified cell.

In some embodiments, said introducing further comprising introducing an at least one additional heterologous gene into said cell, said heterologous gene selected from the group consisting of the group encoding a saponin beta-amyrin synthase, a cytochrome P450, a glycosyltransferase, an acyltransferase, and a UDP-glucose 6-dehydrogenase I, or any combination thereof, wherein said at least one additional heterologous gene is optionally comprised in a vector; and further comprising expressing said at least one additional heterologous gene in said cell. In some embodiments, said at least one heterologous gene is operably linked to a promoter, a transcription termination sequence, or a combination thereof, or said at least one additional heterologous gene is operably linked to a promoter, a transcription termination sequence, or a combination thereof, or a combination thereof of (a) and (b).

In some embodiments, said introducing comprises transforming said at least one cell with said at least one heterologous gene or a polynucleotide sequence encoding said at least one heterologous gene, or the vector comprising said at least one heterologous gene; or said at least one additional heterologous gene or a polynucleotide sequences encoding said at least one additional heterologous gene, or the vector comprising said at least one additional heterologous gene; or a combination thereof of (a) and (b); wherein said expressing comprises transient expression or constitutive expression.

Cellulose synthase like G nucleic acid sequences encoding a CSLG enzyme are described in detail throughout and exemplified in the Examples. In some embodiments, the nucleic acid sequence encoding said at least one heterologous CSLG gene is set forth in any one of SEQ ID NOS: 125, 127, 30, 32, 34, 36, 38, 40, 65, 80, 93, 95, 97, 99, 101, 103, or 105; or a homolog thereof having at least 55% identity to and at least 55% coverage of, the nucleic acid sequence set forth in any one of SEQ ID NOS: 125, 127, 30, 32, 34, 36, 38, 40, 65, 80, 93, 95, 97, 99, 101, 103, or 105. Cellulose synthase like G nucleic acid sequences encoding a CSLG enzyme are described in detail throughout and exemplified in the Examples. In some embodiments, the nucleic acid sequence encoding said at least one heterologous CSLG gene is set forth in any one of SEQ ID NOS: 125, 127, 30, 32, 34, 36, 38, 40, 65, 80, 93, 95, 97, 99, 101, 103, or 105; or a homolog thereof having at least 55% identity to and at least 80% coverage of, the nucleic acid sequence set forth in any one of SEQ ID NOS: 125, 127, 30, 32, 34, 36, 38, 40, 65, 80, 93, 95, 97, 99, 101, 103, or 105.

Cellulose synthase like G nucleic acid sequences encoding a CSLG enzyme are described in detail throughout and exemplified in the Examples. In some embodiments, the nucleic acid sequence encoding said at least one heterologous CSLG gene is set forth in any one of SEQ ID NOS: 125, 127, 30, 32, 34, 36, 38, 40, 65, 80, 93, 95, 97, 99, 101, 103, or 105; or a homolog thereof having at least 80% identity to and at least 80% coverage of, the nucleic acid sequence set forth in any one of SEQ ID NOS: 125, 127, 30, 32, 34, 36, 38, 40, 65, 80, 93, 95, 97, 99, 101, 103, or 105.

In some embodiments, in a method of producing a triterpenoid saponin, the amino acid sequence of said encoded CSLG enzyme is set forth in any one of SEQ ID NOS: 126, 31, 33, 35, 37, 39, 41, 66, 81, 94, 96, 98, 100, 102 or 104; or a homolog thereof having at least 55% identity to and at least 55% coverage of, the amino acid sequence set forth in any one of SEQ ID NOS: 126, 31, 33, 35, 37, 39, 41, 66, 81, 94, 96, 98, 100, 102 or 104. In some embodiments, in a method of producing a triterpenoid saponin, the amino acid sequence of said encoded CSLG enzyme is set forth in any one of SEQ ID NOS: 126, 31, 33, 35, 37, 39, 41, 66, 81, 94, 96, 98, 100, 102 or 104; or a homolog thereof having at least 55% identity to and at least 80% coverage of, the amino acid sequence set forth in any one of SEQ ID NOS: 126, 31, 33, 35, 37, 39, 41, 66, 81, 94, 96, 98, 100, 102 or 104.

In some embodiments, in a method of producing a triterpenoid saponin, the amino acid sequence of said encoded CSLG enzyme is set forth in any one of SEQ ID NOS: 126, 31, 33, 35, 37, 39, 41, 66, 81, 94, 96, 98, 100, 102 or 104; or a homolog thereof having at least 80% identity to and at least 80% coverage of, the amino acid sequence set forth in any one of SEQ ID NOS: 126, 31, 33, 35, 37, 39, 41, 66, 81, 94, 96, 98, 100, 102 or 104.

The GAME proteins, GAME1 and GAME4, were used as baits in potato or tomato, as described herein in the Examples. Genes that were co-expressed with GAME1 or GAME4 are shown in Tables 5-8, and the enzymes of the steroidal alkaloid pathway, along with a portion of the enzymes of steroidal saponin pathway, are presented in FIG. 1 and described in detail throughout and exemplified in the Examples. The SOAP nucleic acid sequences respectively encoding the SOAP enzymes of the triterpenoid saponin pathway presented in FIG. 20A, are described in detail throughout and exemplified in the Examples.

In some embodiments, in a method of producing a steroidal alkaloid, a steroidal saponin, or a triterpenoid saponin, the nucleic acid sequence encoding said at least one additional heterologous gene encodes a β-amyrin synthase, said nucleic acid sequence set forth in SEQ ID NO: 45; or a homolog thereof having at least 80% identity to and at least 80% coverage of, the nucleic acid sequence set forth in SEQ ID NO: 45; or a cytochrome P450, said nucleic acid sequence set forth in any one of SEQ ID NO: 46, 51, or 53; or a homolog thereof having at least 80% identity to and at least 80% coverage of, the nucleic acid sequence set forth in any one of SEQ ID NO: 46, 51, or 53; or a glycosyl transferase, said nucleic acid sequence set forth in any one of SEQ ID NO: 55, 57, 59, 61; or a homolog thereof having at least 80% identity to and at least 80% coverage of, the nucleic acid sequence set forth in any one of SEQ ID NO: 55, 57, 59, or 61; or an acyltransferase, said nucleic acid sequence set forth in SEQ ID NO: 63; or a homolog thereof having at least 80% identity to and at least 80% coverage of, the nucleic acid sequence set forth in SEQ ID NO: 63; or a UDP-glucose 6-dehydrogenase 1, said nucleic acid sequence set forth in SEQ ID NO: 74; or a homolog thereof having at least 80% identity to and at least 80% coverage of, the nucleic acid sequence set forth in SEQ ID NO: 74; or any combination thereof.

In some embodiments, in a method of producing a steroidal alkaloid, a steroidal saponin, or a triterpenoid saponin, the amino acid sequence of said encoded at least one additional heterologous gene encodes a β-amyrin synthase, said amino acid sequence set forth in SEQ ID NO: 48; or a homolog thereof having at least 80% identity to and at least 80% coverage of, the amino acid 214I sequence set forth in SEQ ID NO: 48; or a cytochrome P450, said amino acid sequence set forth in any one of SEQ ID NO: 49, 52, or 54; or a homolog thereof having at least 80% identity to and at least 80% coverage of, the amino acid sequence set forth in any one of SEQ ID NO: 49, 52, or 54; or a glycosyl transferase, said amino acid sequence set forth in any one of SEQ ID NO: 56, 58, 60, or 62; or a homolog thereof having at least 80% identity to and at least 80% coverage of, the amino acid sequence set forth in any one of SEQ ID NO: 56, 58, 60, or 62; or an acyltransferase, said amino acid sequence set forth in SEQ ID NO: 64; or a homolog thereof having at least 80% identity to and at least 80% coverage of, the amino acid sequence set forth in SEQ ID NO: 64; or a UDP-glucose 6-dehydrogenase 1, said amino acid sequence set forth in SEQ ID NO: 75; or a homolog thereof having at least 80% identity to and at least 80% coverage of, the amino acid sequence set forth in SEQ ID NO: 75; or any combination thereof.

In some embodiments, in a method of producing a steroidal alkaloid or steroidal saponin, said steroidal alkaloid or steroidal saponin comprises an anti-nutritive agent, a cosmetic agent, or a pharmaceutical agent or any combination thereof. In some embodiments, in a method of producing a steroidal alkaloid or steroidal saponin, said steroidal alkaloid or steroidal saponin is selected from an anti-nutritive agent, a cosmetic agent, or a pharmaceutical agent, or any combination thereof.

In some embodiments, in a method of producing a triterpenoid saponin, said triterpenoid saponin comprises a sweetener, foaming agent, emulsifier, preservative, anti-carcinogen, hypocholesterolemic agent, anti-inflammatory agent, anti-oxidant, biological adjuvant, anti-microbial agent, insecticidal agent, antifeedant, or an anti-fungal agent, or any combination thereof. In some embodiments, in a method of producing a triterpenoid saponin, said triterpenoid saponin is selected from a sweetener, foaming agent, emulsifier, preservative, anti-carcinogen, hypocholesterolemic agent, anti-inflammatory agent, anti-oxidant, biological adjuvant, anti-microbial agent, insecticidal agent, antifeedant, or anti-fungal agent, or any combination thereof.

In some embodiments, in a method of producing a steroidal alkaloid, said steroidal alkaloid comprises alpha-tomatine, tomatine, alpha-chaconine, alpha-solanine, alpha-solasonine, alpha-solmargine, or any combination thereof. In some embodiments, iun a method of producing a steroidal alkaloid, said steroidal alkaloid is selected from the group consisting of alpha-tomatine, tomatine, alpha-chaconine, alpha-solanine, alpha-solasonine, alpha-solmargine, or any combination thereof.

In some embodiments, in a method of producing a steroidal saponin, said steroidal saponin comprises uttroside B, a tomatoside, or any combination thereof. In some embodiments, iun a method of producing a steroidal alkaloid, said steroidal alkaloid is selected from the group consisting of uttroside B, a tomatoside, or any combination thereof.

In some embodiments, in a method of producing a triterpenoid saponin, said triterpenoid saponin comprises medicagenic acid 3-O-glucuronide (MA-3-GlcA) (Compound 6), Compound 7, Compound 8, Compound 9, Compound 10, Compound 11, glycyrrhizin (Compound 14), Glycyrrhetinic acid 3-O-monoglucuronide (compound 15), bayogenin (Compound 25), bayogenin-hexA-hex-hex (Compound 31), serjanic acid (Compound 26), serjanic acid-hexA-hex (Compound 32), soyasapogenol A (Compound 29), soyasapogenol B (Compound 30), soyasapogenol A-hexA-hex-pent (Compound 34), soyasaponin VI (Compound 35), betavulgaroside IV (Compound 33), hederagenin-3GlcA, gypsogenin-3GlcA, gypsogenic acid-3GlcA, or a QS-21 adjuvant, or any combination thereof. In some embodiments, in a method of producing a triterpenoid saponin, said triterpenoid saponin is selected from medicagenic acid 3-O-glucuronide (MA-3-GlcA) (Compound 6), Compound 7, Compound 8, Compound 9, Compound 10, Compound 11, glycyrrhizin (Compound 14), Glycyrrhetinic acid 3-O-monoglucuronide (compound 15), bayogenin (Compound 25), bayogenin-hexA-hex-hex (Compound 31), serjanic acid (Compound 26), serjanic acid-hexA-hex (Compound 32), soyasapogenol A (Compound 29), soyasapogenol B (Compound 30), soyasapogenol A-hexA-hex-pent (Compound 34), soyasaponin VI (Compound 35), soyasaponin I (Compound 36), betavulgaroside IV (Compound 33), hederagenin-3GlcA, gypsogenin-3GlcA, gypsogenic acid-3GlcA, or a QS-21 adjuvant, or any combination thereof.

In some embodiments, derivatives of steroidal alkaloids, steroidal saponins, or triterpenoid saponins comprise glycosylated derivatives of, respectively, steroidal alkaloids, steroidal saponins, or triterpenoid saponins.

In some embodiments, in a method of producing a steroidal alkaloid, a steroidal saponin, or a triterpenoid saponin, said cell comprises a plant cell, a yeast cell, an alga cell, an insect cell, or a bacterium. In some embodiments, in a method of producing a steroidal alkaloid, a steroidal saponin, or a triterpenoid saponin, said cell comprises a plant cell, a yeast cell, or an alga cell. In some embodiments, in a method of producing a steroidal alkaloid, a steroidal saponin, or a triterpenoid saponin, said cell comprises a plant cell or a yeast cell. In some embodiments, in a method of producing a steroidal alkaloid, a steroidal saponin, or a triterpenoid saponin, said cell comprises a plant cell. In some embodiments, in a method of producing a steroidal alkaloid, a steroidal saponin, or a triterpenoid saponin, said cell comprises a yeast cell. In some embodiments, in a method of producing a steroidal alkaloid, a steroidal saponin, or a triterpenoid saponin, said cell comprises an alga cell. In some embodiments, in a method of producing a steroidal alkaloid, a steroidal saponin, or a triterpenoid saponin, said cell comprises an insect cell. In some embodiments, in a method of producing a steroidal alkaloid, a steroidal saponin, or a triterpenoid saponin, said cell comprises a bacterium.

In some embodiments, the at least one heterologous gene is operably linked to a promoter, a transcription termination sequence, or a combination thereof.

In some embodiments, the step of introducing comprises transforming said at least one cell with said polynucleotide sequence or a vector comprising the polynucleotide sequence, and wherein said expression comprises transient expression or constitutive expression. In some embodiments, the vector comprises an expression vector. In some embodiments, the vector comprises a plasmid vector. In some embodiments, the vector integrates into the host cell DNA. In some embodiments, part of the polynucleotide comprised in the vector integrates into the host cell DNA. In some embodiments, the vector does not integrate into the host cell DNA and replicates as a separate entity within the host cell.

In some embodiments, expression vectors used to produce genetically modified plant cells include both Agrobacterium and non-Agrobacterium vectors. Agrobacterium-mediated gene transfer exploits the natural ability of Agrobacterium tumefaciens to transfer DNA into plant chromosomes and is described in detail in G. Gheysen, G. Angenon, and M. Van Montagu, 1998, Agrobacterium-mediated plant transformation: a scientifically intriguing story with significant applications in K. Lindsey (Ed.), Transgenic Plant Research, Harwood Academic Publishers, Amsterdam, pp. 1-33; and in H. A. Stafford (2000), Botanical Review 66:99-118. A second group of transformation methods is the non-Agrobacterium-mediated transformation and these methods are known as direct gene transfer methods. An overview is brought by P. Barcelo and P. A. Lazzeri (1998), Direct gene transfer: chemical, electrical and physical methods in K. Lindsey (Ed.), Transgenic Plant Research, Harwood Academic Publishers, Amsterdam, pp. 35-55. Methods include particle gun delivery, microinjection, electroporation of intact cells, polyethyleneglycol-mediated protoplast transformation, electroporation of protoplasts, liposome-mediated transformation, silicon-whiskers-mediated transformation, etc. A suitable control plant would include a genetically unaltered or non-transgenic plant of the parental line used to generate a transgenic plant herein.

Genetically transformed hairy root cultures can be obtained by transformation with virulent strains of Agrobacterium rhizogenes. Protocols used for establishing of hairy root cultures vary, as well as the susceptibility of plant species to infection by Agrobacterium (Toivounen et al. 1993; Vanhala et al. 1995). It is known that the Agrobacterium strain used for transformation has a great influence on root morphology and the degree of secondary metabolite accumulation in hairy root cultures. It is possible by systematic clone selection, e.g., via protoplasts, to find high yielding, stable, and from single-cell-derived hairy root clones. This is possible because the hairy root cultures possess a great somaclonal variation. Another possibility of transformation is the use of viral vectors (Turpen 1999).

Any plant tissue or plant cells capable of subsequent clonal propagation, whether by organogenesis or embryogenesis, may be transformed with an expression vector of interest. The team “organogenesis” means a process by which shoots and roots are developed sequentially from meristematic centers; the term “embryogenesis” means a process by which shoots and roots develop together in a concerted fashion (not sequentially), whether from somatic cells or gametes. The particular tissue chosen will vary depending on the clonal propagation systems available for, and best suited to, the particular species being transformed. Exemplary tissue targets include protoplasts, leaf disks, pollen, embryos, cotyledons, hypocotyls, megagametophytes, callus tissue, existing meristematic tissue (e.g., apical meristems, axillary buds, and root meristems), and induced meristem tissue (e.g., cotyledon meristem and hypocotyls meristem).

In some embodiments, disclosed herein is a vector comprising a polynucleotide sequence or at least a heterologous gene encoding an enzyme as described in detail above. In some embodiments, disclosed herein is a host cell, for example but not limited to a plant cell, a yeast, or an algal cell, comprising a polynucleotide sequence or at least a heterologous gene encoding an enzyme as described in detail above, or a vector comprising a polynucleotide or at least one heterologous gene encoding an enzyme as described throughout.

The term “vector” in certain embodiments, encompasses a nucleic acid molecule capable of transporting another nucleic acid molecule to which it has been operably or continuously linked. The vector may be of any suitable type including, but not limited to, a phage, virus, plasmid, phagemid, cosmid, bacmid or even an artificial chromosome. Certain vectors are capable of autonomous replication in a host cell into which they are introduced (e.g., vectors having an origin of replication that functions in the host cell). Other vectors can be integrated into the genome of a host cell upon introduction into the host cell, and are thereby replicated along with the host genome. Moreover, in some embodiments, vectors are capable of directing the expression of certain genes of interest. Such vectors are referred to herein as “recombinant expression vectors” (or simply, “expression vectors”). Suitable vectors have regulatory sequences, such as promoters, enhancers, terminator sequences, and the like as desired and according to a particular host organism (e.g., plant cell, yeast cell, or algal cell).

Typically, a recombinant vector according to the disclosure comprises at least one “heterologous gene encoding an enzyme” or “expression cassette comprising an at least one heterologous gene encoding an enzyme”. Expression cassettes are generally DNA constructs, which in some embodiments include (5′ to 3′ in the direction of transcription): a promoter region, a polynucleotide sequence comprising at least one heterologous gene encoding an enzyme, homologue, variant, or fragment thereof of the disclosure as described above in detail, operably linked with the transcription initiation region, and a termination sequence including a stop signal for RNA polymerase and a polyadenylation signal. It is understood that all of these regions should be capable of operating in biological cells, such as plant cells or yeast cells or algal cells, to be transformed. The promoter region comprising the transcription initiation region, which preferably includes the RNA polymerase binding site, and the polyadenylation signal may be native to the biological cell to be transformed or may be derived from an alternative source, where the region is functional in the biological cell.

The term “recombinant host cell” (“expression host cell,” “expression host system,” “expression system” or simply “host cell”), as used herein, encompasses a cell into which a recombinant vector has been introduced. It should be understood that such terms are intended to refer not only to the particular subject cell but to the progeny of such a cell. Because certain modifications may occur in succeeding generations due to either mutation or environmental influences, such progeny may not, in fact, be identical to the parent cell, but are still included within the scope of the term “host cell” as used herein. A recombinant host cell may be an isolated cell or cell line grown in culture or may be a cell that resides in a living tissue or organism. Host cells can be of fungal, plant or algal origin.

In some embodiments, disclosed herein is a transgenic plant or a cell derived thereof that is transformed with the above-described vector.

In some embodiments in methods of producing a steroidal alkaloid, a steroidal saponin, or a triterpenoid saponin, the encoded enzyme comprises a saponin beta-amyrin synthase, a cytochrome P450, a glycosyltransferase, an acyltransferase, a UDP-glucose 6-dehydrogenase I, or a cellulose synthase like G (CSLG). In some embodiments in methods of producing a steroidal alkaloid, a steroidal saponin, or a triterpenoid saponin, multiple enzymes are encoded by heterologous genes wherein the encoded enzymes comprises a combination of a saponin beta-amyrin synthase, a cytochrome P450, a glycosyltransferase, an acyltransferase, a UDP-glucose 6-dehydrogenase I, or a cellulose synthase like G (CSLG). In some embodiments in methods of producing a steroidal alkaloid, a steroidal saponin, or a triterpenoid saponin, 1, 2, 3, 4, 5, 6, 7, 8, 9, 10, or more enzymes are encoded by heterologous genes wherein the encoded enzymes comprises a combination of a saponin beta-amyrin synthase, a cytochrome P450, a glycosyltransferase, an acyltransferase, a UDP-glucose 6-dehydrogenase I, or a cellulose synthase like G (CSLG). In some embodiments in methods of producing a steroidal alkaloid, a steroidal saponin, or a triterpenoid saponin, the encoded enzyme comprises a CSLG.

In some embodiments, the cytochrome P450 comprises a C2-hydroxylase or is a C-23 oxidase. In some embodiments, the glycosyltransferase comprises a fructosyltransferase, a xylosyltransferase, or a UDP-glycosyltransferase, or a combination thereof. In some embodiments, the acyltransferase comprises a benzylalcohol acetyl-, anthocyanin-O-hydroxy-cinnamoyl-, anthranilate-N-hydroxy-cinnamyol/benzoyl-, deacetylvindoline (BAHD) acetyletransferase. In some embodiments, in a method of producing a triterpenoid saponin the encoded cytochrome P450 comprises a C2-hydroxylase or is a C-23 oxidase, or a combination thereof, said glycosyltransferase comprises a fructosyltransferase, a xylosyltransferase, or a UDP-glycosyltransferase, or a combination thereof, the said acyltransferase comprise a benzylalcohol acetyl-, anthocyanin-O-hydroxy-cinnamoyl-, anthranilate-N-hydroxy-cinnamyol/benzoyl-, deacetylvindoline (BAHD) acetyletransferase; or the encoded enzymes comprise a combination of any of these enzymes, wherein optionally additional enzymes are comprised within a polynucleotide sequence or sequences.

In some embodiments in a method of producing a steroidal alkaloid, a steroidal saponin, or a triterpenoid saponin, the polynucleotide sequence of said at least one heterologous gene is set forth in any one of SEQ ID NOS: 125, 127, 30, 32, 34, 36, 38, 40, 45, 46, 47, 51, 53, 55, 57, 59, 61, 63, 65, 80, 93, 95, 97, 99, 101, 103, or 105; or the polynucleotide sequence of said at least one heterologous gene comprises a nucleic acid sequence having at least 55% identity to the nucleic acid sequence set forth in any one of SEQ ID NOS: 125, 127, 30, 32, 34, 36, 38, 40, 45, 46, 47, 51, 53, 55, 57, 59, 61, 63, 65, 80, 93, 95, 97, 99, 101, 103, or 105. In some embodiments, wherein multiple heterologous genes are expressed, the skilled artisan would appreciate that a polynucleotide sequence or sequences may comprise a combination of any of SEQ ID NOS: 125, 127, 30, 32, 34, 36, 38, 40, 45, 46, 47, 51, 53, 55, 57, 59, 61, 63, 65, 80, 93, 95, 97, 99, 101, 103, or 105, as described in detail above. In some embodiments, wherein multiple heterologous genes are expressed, the skilled artisan would appreciate that a polynucleotide sequence or sequences may comprise a combination of comprises a nucleic acid sequence having at least 55% identity to the nucleic acid sequence set forth in any one of SEQ ID NOS: 125, 127, 30, 32, 34, 36, 38, 40, 45, 46, 47, 51, 53, 55, 57, 59, 61, 63, 65, 80, 93, 95, 97, 99, 101, 103, or 105.

In some embodiments in a method of producing a steroidal alkaloid, a steroidal saponin, or a triterpenoid saponin, the polynucleotide sequence of said at least one heterologous gene is set forth in any one of SEQ ID NOS: 125, 127, 30, 32, 34, 36, 38, 40, 45, 46, 47, 51, 53, 55, 57, 59, 61, 63, 65, 80, 93, 95, 97, 99, 101, 103, or 105; or the polynucleotide sequence of said at least one heterologous gene comprises a nucleic acid sequence having at least 80% identity to the nucleic acid sequence set forth in any one of SEQ ID NOS: 125, 127, 30, 32, 34, 36, 38, 40, 45, 46, 47, 51, 53, 55, 57, 59, 61, 63, 65, 80, 93, 95, 97, 99, 101, 103, or 105. In some embodiments, wherein multiple heterologous genes are expressed, the skilled artisan would appreciate that a polynucleotide sequence or sequences may comprise a combination of any of SEQ ID NOS: 125, 127, 30, 32, 34, 36, 38, 40, 45, 46, 47, 51, 53, 55, 57, 59, 61, 63, 65, 80, 93, 95, 97, 99, 101, 103, or 105, as described in detail above. In some embodiments, wherein multiple heterologous genes are expressed, the skilled artisan would appreciate that a polynucleotide sequence or sequences may comprise a combination of comprises a nucleic acid sequence having at least 80% identity to the nucleic acid sequence set forth in any one of SEQ ID NOS: 125, 127, 30, 32, 34, 36, 38, 40, 45, 46, 47, 51, 53, 55, 57, 59, 61, 63, 65, 80, 93, 95, 97, 99, 101, 103, or 105.

In some embodiments in a method of producing a steroidal alkaloid, a steroidal saponin, or a triterpenoid saponin, the polynucleotide sequence of said at least one heterologous gene encodes the amino acid sequence of said enzyme set forth in any one of SEQ ID NOS: 126, 31, 33, 35, 37, 39, 41, 48, 49, 50, 52, 54, 56, 58, 60, 62, 64, 64, 81, 94, 96, 98, 100, 102 or 104; or the polynucleotide sequence of said at least one heterologous gene encodes the amino acid sequence of said enzyme set forth in the sequence having at least 55% identity to the amino acid sequence set forth in any one of SEQ ID NOS: 126, 31, 33, 35, 37, 39, 41, 48, 49, 50, 52, 54, 56, 58, 60, 62, 64, 64, 81, 94, 96, 98, 100, 102 or 104.

In some embodiments in a method of producing a steroidal alkaloid, a steroidal saponin, or a triterpenoid saponin, the polynucleotide sequence of said at least one heterologous gene encodes the amino acid sequence of said enzyme set forth in any one of SEQ ID NOS: 126, 31, 33, 35, 37, 39, 41, 48, 49, 50, 52, 54, 56, 58, 60, 62, 64, 64, 81, 94, 96, 98, 100, 102 or 104; or the polynucleotide sequence of said at least one heterologous gene encodes the amino acid sequence of said enzyme set forth in the sequence having at least 80% identity to the amino acid sequence set forth in any one of SEQ ID NOS: 126, 31, 33, 35, 37, 39, 41, 48, 49, 50, 52, 54, 56, 58, 60, 62, 64, 64, 81, 94, 96, 98, 100, 102 or 104.

In some embodiments in a method of producing a steroidal alkaloid, a steroidal saponin, or a triterpenoid saponin, the genetically modified cell, for example but not limited to a plant cell, a yeast, or an algal cell comprises an increased content of at least a steroidal alkaloid, a steroidal saponin, or a triterpenoid saponin, compared to a corresponding unmodified cell. In some embodiments in a method of producing a steroidal alkaloid, a steroidal saponin, or a triterpenoid saponin, the genetically modified cell, for example but not limited to a plant cell, a yeast, an algal cell, an insect cell, or a bacterium, comprises an increased content of at least a steroidal alkaloid, a steroidal saponin, or a triterpenoid saponin, compared to a corresponding unmodified cell. In some embodiments in a method of producing a steroidal alkaloid, a steroidal saponin, or a triterpenoid saponin, the genetically modified cell comprises a plant cell comprising an increased content of at least a steroidal alkaloid, a steroidal saponin, or a triterpenoid saponin, compared to a corresponding unmodified cell. In some embodiments in a method of producing a steroidal alkaloid, a steroidal saponin, or a triterpenoid saponin, the genetically modified cell comprises a yeast cell comprising an increased content of at least a steroidal alkaloid, a steroidal saponin, or a triterpenoid saponin, compared to a corresponding unmodified cell. In some embodiments in a method of producing a steroidal alkaloid, a steroidal saponin, or a triterpenoid saponin, the genetically modified cell comprises a algal cell comprising an increased content of at least a steroidal alkaloid, a steroidal saponin, or a triterpenoid saponin, compared to a corresponding unmodified cell. In some embodiments in a method of producing a steroidal alkaloid, a steroidal saponin, or a triterpenoid saponin, the genetically modified cell comprises a plant cell comprising an increased content of at least a steroidal alkaloid, a steroidal saponin, or a triterpenoid saponin, compared to a corresponding unmodified cell, wherein said plant cell is comprised in a plant or plant part.

In some embodiments, in a method of producing a steroidal alkaloid, the steroidal alkaloid produced comprises an anti-nutritive agent, a cosmetic agent, or a pharmaceutical agent, or any combination thereof, or the steroidal alkaloid comprises an esculeoside or dehydroesculeoside; or the steroidal alkaloid comprises alpha-tomatine, tomatine, dehydrotomatine, alpha-chaconine, alpha-solanine, alpha-solasonine, alpha-solmargine, or any combination thereof. In some embodiments, in a method of producing a steroidal alkaloid, the steroidal alkaloid produced comprises alpha-tomatine, tomatine, dehydrotomatine, alpha-chaconine, alpha-solanine, alpha-solasonine, alpha-solmargine, or any combination thereof.

In some embodiments, in a method of producing a steroidal alkaloid, the steroidal alkaloid produced comprises alpha-tomatine. In some embodiments, in a method of producing a steroidal alkaloid, the steroidal alkaloid produced comprises tomatine. In some embodiments, in a method of producing a steroidal alkaloid, the steroidal alkaloid produced comprises dehydrotomatine. In some embodiments, in a method of producing a steroidal alkaloid, the steroidal alkaloid produced comprises alpha-chaconine. In some embodiments, in a method of producing a steroidal alkaloid, the steroidal alkaloid produced comprises alpha-solanine. In some embodiments, in a method of producing a steroidal alkaloid, the steroidal alkaloid produced comprises alpha-solasonine. In some embodiments, in a method of producing a steroidal alkaloid, the steroidal alkaloid produced comprises alpha-solmargine.

In some embodiments, in a method of producing a steroidal saponin, the steroidal saponin produced comprises an anti-nutritive agent, a cosmetic agent, or a pharmaceutical agent, or any combination thereof, or the steroidal alkaloid comprises uttroside B, a tomatoside, or any combination thereof. In some embodiments, in a method of producing a steroidal saponin, the steroidal saponin produced comprises uttroside B, a tomatoside, or any combination thereof.

In some embodiments, in a method of producing a steroidal saponin, the steroidal saponin produced comprises uttroside B. In some embodiments, in a method of producing a steroidal saponoin, the steroidal saponin produced comprises a tomatoside.

In some embodiments in a method of producing a triterpenoid saponin, the triterpenoid saponin produced comprises a sweetener, foaming agent, emulsifier, preservative, anti-carcinogen, hypocholesterolemic agent, anti-inflammatory agent, anti-oxidant, biological adjuvant, anti-microbial agent, insecticidal agent, antifeedant, anti-fungal agent, or any combination thereof, or the triterpenoid saponin comprises medicagenic acid 3-O-glucuronide (MA-3-GlcA) (Compound 6), Compound 7, Compound 8, Compound 9, Compound 10, Compound 11, glycyrrhizin (Compound 14), Glycyrrhetinic acid 3-O-monoglucuronide (compound 15), bayogenin (Compound 25), bayogenin-hexA-hex-hex (Compound 31), serjanic acid (Compound 26), serjanic acid-hexA-hex (Compound 32), soyasapogenol A (Compound 29), soyasapogenol B (Compound 30), soyasapogenol A-hexA-hex-pent (Compound 34), soyasaponin VI (Compound 35), betavulgaroside IV (Compound 33), hederagenin-3GlcA, gypsogenin-3GlcA, gypsogenic acid-3GlcA, or a QS-21 adjuvant, or any combination thereof; or a combination thereof. In some embodiments in a method of producing a triterpenoid saponin, the triterpenoid saponin produced comprises medicagenic acid 3-O-glucuronide (MA-3-GlcA) (Compound 6), Compound 7, Compound 8, Compound 9, Compound 10, Compound 11, glycyrrhizin (Compound 14), Glycyrrhetinic acid 3-O-monoglucuronide (compound 15), bayogenin (Compound 25), bayogenin-hexA-hex-hex (Compound 31), serjanic acid (Compound 26), serjanic acid-hexA-hex (Compound 32), soyasapogenol A (Compound 29), soyasapogenol B (Compound 30), soyasapogenol A-hexA-hex-pent (Compound 34), soyasaponin VI (Compound 35), soyasaponin I (Compound 36), betavulgaroside IV (Compound 33), hederagenin-3GlcA, gypsogenin-3GlcA, gypsogenic acid-3GlcA, or a QS-21 adjuvant, or any combination thereof.

In some embodiments in a method of producing a triterpenoid saponin, the triterpenoid saponin produced comprises medicagenic acid 3-O-glucuronide (MA-3-GlcA) (Compound 6). In some embodiments in a method of producing a triterpenoid saponin, the triterpenoid saponin produced comprises Compound 7. In some embodiments in a method of producing a triterpenoid saponin, the triterpenoid saponin produced comprises Compound 8. In some embodiments in a method of producing a triterpenoid saponin, the triterpenoid saponin produced comprises Compound 9. In some embodiments in a method of producing a triterpenoid saponin, the triterpenoid saponin produced comprises Compound 10. In some embodiments in a method of producing a triterpenoid saponin, the triterpenoid saponin produced comprises Compound 11 In some embodiments in a method of producing a triterpenoid saponin, the triterpenoid saponin produced comprises glycyrrhizin (Compound 14). In some embodiments in a method of producing a triterpenoid saponin, the triterpenoid saponin produced comprises Glycyrrhetinic acid 3-O-monoglucuronide (compound 15). In some embodiments in a method of producing a triterpenoid saponin, the triterpenoid saponin produced comprises bayogenin (Compound 25). In some embodiments in a method of producing a triterpenoid saponin, the triterpenoid saponin produced comprises bayogenin-hexA-hex-hex (Compound 31). In some embodiments in a method of producing a triterpenoid saponin, the triterpenoid saponin produced comprises serjanic acid (Compound 26). In some embodiments in a method of producing a triterpenoid saponin, the triterpenoid saponin produced comprises serjanic acid-hexA-hex (Compound 32). In some embodiments in a method of producing a triterpenoid saponin, the triterpenoid saponin produced comprises soyasapogenol A (Compound 29). In some embodiments in a method of producing a triterpenoid saponin, the triterpenoid saponin produced comprises soyasapogenol B (Compound 30). In some embodiments in a method of producing a triterpenoid saponin, the triterpenoid saponin produced comprises soyasapogenol A-hexA-hex-pent (Compound 34). In some embodiments in a method of producing a triterpenoid saponin, the triterpenoid saponin produced comprises soyasaponin VI (Compound 35). In some embodiments in a method of producing a triterpenoid saponin, the triterpenoid saponin produced comprises soyasaponin I (Compound 36). In some embodiments in a method of producing a triterpenoid saponin, the triterpenoid saponin produced comprises betavulgaroside IV (Compound 33). In some embodiments in a method of producing a triterpenoid saponin, the triterpenoid saponin produced comprises hederagenin-3GlcA, gypsogenin-3GlcA. In some embodiments in a method of producing atriterpenoid saponin, the triterpenoid saponin produced comprises gypsogenic acid-3GlcA. In some embodiments in a method of producing a triterpenoid saponin, the triterpenoid saponin produced comprises a QS-21 adjuvant, or any combination thereof.

In some embodiments, a method of producing a steroidal alkaloid, steroidal saponin, or triterpenoid saponin comprises increasing the content of the steroidal alkaloid, steroidal saponin, or triterpenoid saponin, respectively, compared to an unmodified cell or an unmodified plant and altering the content of a phytosterol, or a derivative thereof, a metabolite thereof, or a biosynthetic intermediate thereof, a cholesterol, or a derivative thereof, a metabolite thereof, or a biosynthetic intermediate thereof, a phytocholesterol, or a derivative thereof, a metabolite thereof, or a biosynthetic intermediate thereof, a cholestenol, or a derivative thereof, a metabolite thereof, or a biosynthetic intermediate thereof, or a phytocholestenol, or a derivative thereof, a metabolite thereof, or a biosynthetic intermediate thereof.

In some embodiments, a method of producing a steroidal alkaloid, steroidal saponin, or triterpenoid saponin comprises increasing the content of the steroidal alkaloid, steroidal saponin, or triterpenoid saponin, respectively, compared to an unmodified cell or an unmodified plant and decreasing the content of a phytosterol, or a derivative thereof, a metabolite thereof, or a biosynthetic intermediate thereof; a cholesterol, or a derivative thereof, a metabolite thereof, or a biosynthetic intermediate thereof, a phytocholesterol, or a derivative thereof, a metabolite thereof, or a biosynthetic intermediate thereof, a cholestenol, or a derivative thereof, a metabolite thereof, or a biosynthetic intermediate thereof, or a phytocholestenol, or a derivative thereof, a metabolite thereof, or a biosynthetic intermediate thereof.

In some embodiments in a method of producing a steroidal alkaloid, a steroidal saponin, or a triterpenoid saponin, the cell into which the polynucleotide sequence is introduced comprises a plant cell or a yeast cell. Alternatively, in some embodiments, it comprises an algal cell. Plant and yeast cells that may be genetically modified as described herein, have been disclosed above in detail. Those same plant and yeast cells may, in some embodiments, be used in a method of producing a steroidal alkaloid, a steroidal saponin, or a triterpenoid saponin. In some embodiments in a method of producing a steroidal alkaloid, a steroidal saponin, or a triterpenoid saponin, the cell into which the polynucleotide sequence is introduced comprises a plant cell or a yeast cell or an algal cell or an insect cell or a bacterium.

In some embodiments in a method of producing a steroidal alkaloid, a steroidal saponin, or a triterpenoid saponin, the cell comprises a plant cell that comprises a leaf cell, a petiole cell, a plant stem or stalk cell, a root cell, a bud cell, a tuber cell, a bean cell, a grain or kernel cell, a fruit cell, a nut cell, a legume cell, a seed or seed cell, a bract cell, a callus cell, and a flower cell.

In some embodiments in a method of producing a steroidal alkaloid, a steroidal saponin, or a triterpenoid saponin, the yeast cell, the algal cell, or the plant cell is from an order, genus, or species recited herein. For example, but not limited to a plant cell comprising a cell from a plant in the Poales order, the Caryophyllales order, the Solanales order, the Fabales order, the Malvales order, the Apiales order, the Brassicales order, the Asparagales order, the Dioscoreales order, or the Liliales order; or a yeast is selected from a Saccharomyces genus, a Schizosaccharomyces genus, a Pichia genus, a Yarrowia genus, a Kluyveromyces genus, or a Candida genus.

In some embodiments of producing a steroidal alkaloid, a steroidal saponin, or a triterpenoid saponin, the plant of the Caryophyllales order is selected from the group of genera consisting of the Spinacia genus, the Chenopodium genus, the Beta genus, and the Rheum genus; or the plant of the Solanales order is selected from the group of genera consisting of the Nicotiana genus, the Solanum genus, and the Capsicum genus; or the plant of the Fabales order is selected from the group of genera consisting of the Glycyrrhiza genus, the Medicago genus, the Quillaja genus, the Glycine genus, and the Lotus genus; or the plant of the Apiales order is selected from the group of genera consisting of the Panax genus, Daucus genus, the Apium genus, and the Petroselinum genus, or the plant from the Poales order is selected from the group of genera consisting of the Oryza genus (e.g., Oryza sativa and Oryza glaberrima [rice]), the Hordeum genus (e.g., Hordeum vulgare [barley]), the Avena genus (e.g., Avena sativa [oat], Avena strigosa), and the Triticum genus (e.g., Triticum spelta [spelt]), or the plant from the Brassicales order is selected from the group of genera consisting of the Arabidopsis genus (e.g., Arabidopsis thaliana), the Brassica genus (e.g., Brassica oleracea [cabbages], Brassica juncea [white mustard], Brassica nigra [black mustard], Brassica napus), the Capparis genus (e.g., Capparis spinosa [caper]), and the Carica genus (e.g., Carica papaya [papaya]).

In some embodiments in a method of producing a steroidal alkaloid, a steroidal saponin, or a triterpenoid saponin, the plant of the Solanales order is selected from the group of species consisting of Nicotiana benthamiana, Solanum lycopersicum, Solanum tuberosum, Solanum melongena, Solanum pennellii, Solanum chacoense, Solanum dulcamara, and Capsicum annuum; or the plant of the Fabales order is selected from the group of species consisting of Pisum sativum, Glycyrrhiza uralensis, Medicago sativa, Medicago truncatula, Quillaja saponaria, Glycine max, and Lotus japonicus; or the plant of the Malvales order is selected from the Theobroma genus; or the plant of the Apiales order is selected from the group of species consisting of Panax ginseng, Daucus carota, Apium graveolens, and Petroselinum crispum; or the plant is selected from the species Theobroma cacao, or the plant from the Poales order is selected from the group of genera consisting of the Oryza genus (e.g., Oryza sativa and Oryza glaberrima [rice]), the Hordeum genus (e.g., Hordeum vulgare [barley]), the Avena genus (e.g., Avena sativa [oat], Avena strigosa), and the Triticum genus (e.g., Triticum spelta [spelt]), or the plant from the Brassicales order is selected from the group of genera consisting of the Arabidopsis genus (e.g., Arabidopsis thaliana), the Brassica genus (e.g., Brassica oleracea [cabbages], Brassica juncea [white mustard], Brassica nigra [black mustard], Brassica napus), the Capparis genus (e.g., Capparis spinosa [caper]), and the Carica genus (e.g., Carica papaya [papaya]).

In some embodiments in a method of producing a steroidal alkaloid, a steroidal saponin, or a triterpenoid saponin, the yeast is selected from a Saccharomyces genus, a Schizosaccharomyces genus, a Pichia genus, a Yarrowia genus, a Kluyveromyces genus, or a Candida genus. In some embodiments, said yeast is a Saccharomyces cerevisiae or Candida albicans. In some embodiments in a method of producing a steroidal alkaloid, a steroidal saponin, or a triterpenoid saponin, the method further comprises a step of purifying said steroidal alkaloid, steroidal saponin, or triterpenoid saponin.

Methods of purifying steroidal alkaloids, steroidal saponins, or triterpenoids may include standard purifications techniques known in the art, for example but not limited to column liquid chromatography, preparative medium-pressure liquid chromatograph, and preparative HPLC. These techniques may be used in embodiments of purifying steroidal alkaloids, steroidal saponins, or triterpenoid saponins from plants, plant part, or plant cells. Alternatively, the plant cells or plant parts may be used as is without purifying the steroidal alkaloid, steroidal saponin, or triterpenoid saponin.

In some embodiments, when steroidal alkaloids, steroidal saponins, or triterpenoid saponins are produced in yeast cells, the steroidal alkaloids, steroidal saponins, or triterpenoid saponins are secreted from the cells. In some embodiments, a secreted steroidal alkaloid, steroidal saponin, or triterpenoid saponin is purified from a yeast cell medium.

In some embodiments, a steroidal alkaloid, steroidal saponin or triterpenoid saponin is extracted from a plant cell, a plant part, a plant, algae, a yeast, an insect cell, or bacterial culture using methods known in the art.

In some embodiments, provided herein is a method of producing at least one steroidal alkaloid, steroidal saponin, or triterpenoid saponin, the method further comprising the step of extracting said steroidal alkaloid, steroidal saponin, or triterpenoid saponin from the plant, plant part, colony, organ, tissue, or cells that produce said steroidal alkaloid, steroidal saponin, or triterpenoid saponin. In one embodiment, the plant part comprises a leaf, a petiole, a plant stem or stalk, a root, a bud, a tuber, a bean, a grain or kernel, a fruit, a nut, a legume, a seed or seed, a bract, or a flower.

### Methods of Altering the Content of Steroidal Alkaloids, Steroidal Saponins, and Triterpenoid Saponins in a Plant or Plant Part or Plant Cell

In some embodiments, disclosed herein is a method of altering the content of at least one steroidal alkaloid, steroidal saponin, or triterpenoid saponin in a genetically modified plant, a genetically modified plant part, or a genetically modified plant cell, the method comprising genetically modifying the CSLG gene in said plant, plant part, or said cell, wherein said modification comprises increasing expression of said CSLG gene, or increasing activity of the expressed CSLG enzyme, or increasing stability of the expressed CSLG enzyme, or any combination thereof, wherein said plant, plant part, or plant cell comprises an increased content of at least one steroidal alkaloid, steroidal saponin, or triterpenoid saponin compared to a corresponding unmodified plant, plant part, or plant cell. In other embodiments, disclosed herein is a method of altering the content of at least one steroidal alkaloid, steroidal saponin, or triterpenoid saponin in a genetically modified plant, a genetically modified plant part, or a genetically modified plant cell, the method comprising genetically modifying the CSLG gene in said plant, plant part, or said cell, wherein said modification comprises decreasing or eliminating expression of said CSLG gene, or decreasing activity of the expressed CSLG enzyme, or decreasing stability of the expressed CSLG enzyme, or any combination thereof, wherein said plant, plant part, or cell comprises a decreased content of at least one steroidal alkaloid, steroidal saponin, or triterpenoid saponin compared to a corresponding unmodified plant, plant part, or plant cell.

In some embodiments, a genetically modified plant described in detail herein, comprises an altered content of at least one steroidal alkaloid, steroidal saponin, or triterpenoid saponin disclosed herein.

In certain embodiments, disclosed herein is a method of reducing the content of at least one steroidal alkaloid, a derivative thereof, a metabolite thereof, or a biosynthetic intermediate thereof, one steroidal saponin, a derivative thereof, a metabolite thereof, or a biosynthetic intermediate thereof, or one triterpenoid saponin, a derivative thereof, a metabolite thereof, or a biosynthetic intermediate thereof, in at least one cell of a plant or a plant part, the method comprising genetically modifying said at least one plant cell, said genetic modification comprising:
(a) transforming said at least one plant cell with at least one silencing molecule targeted to a nucleic acid gene sequence encoding a Cellulose Synthase Like G (CSLG) enzyme; or
(b) mutagenizing at least one nucleic acid sequence encoding a Cellulose Synthase Like G (CSLG) enzyme, wherein the mutagenesis comprises introducing (1) one or more point mutations into the nucleic acid sequence, (2) deletions within the nucleic acid sequence, or (3) insertions within the nucleic acid, or any combination thereof, wherein said introducing comprising mutagenizing coding or non-coding sequence;
   wherein expression of the gene encoding the CSLG enzyme is reduced in the genetically modified plant cell compared to its expression in a corresponding unmodified plant cell, wherein the plant comprising said genetically modified cell comprises reduced content of one steroidal alkaloid, a derivative thereof, a metabolite thereof, or a biosynthetic intermediate thereof, one steroidal saponin, a derivative thereof, a metabolite thereof, or a biosynthetic intermediate thereof, or at least one triterpenoid saponin, a derivative thereof, a metabolite thereof, or a biosynthetic intermediate thereof, compared to the corresponding unmodified plant.

In certain embodiments, disclosed herein is a method of increasing the content of at least one steroidal alkaloid, a derivative thereof, a metabolite thereof, or a biosynthetic intermediate thereof, at least one steroidal saponin, a derivative thereof, a metabolite thereof, or a biosynthetic intermediate thereof, or at least one triterpenoid saponin, a derivative thereof, a metabolite thereof, or a biosynthetic intermediate thereof, in at least one cell of a plant or plant part, the method comprising genetically modifying said at least one plant cell, said genetic modification comprising:
(a) mutagenizing at least one nucleic acid sequence encoding a Cellulose Synthase Like G (CSLG) enzyme, wherein the mutagenesis comprises introducing (1) one or more point mutations into the nucleic acid sequence, (2) deletions within the nucleic acid sequence, or (3) insertions within the nucleic acid, or any combination thereof, wherein said introducing comprising mutagenizing coding or non-coding sequence; and
(b) expressing said nucleic acid encoding said CSLG; and
   wherein the plant comprising said genetically modified cell comprises increased content of at least one steroidal alkaloid, a derivative thereof, a metabolite thereof, or a biosynthetic intermediate thereof, at least one steroidal saponin, a derivative thereof, a metabolite thereof, or a biosynthetic intermediate thereof, or at least one triterpenoid saponin, a derivative thereof, a metabolite thereof, or a biosynthetic intermediate thereof, compared to the corresponding unmodified plant.

Methods for mutagenizing at least one nucleic acid sequence encoding a steroidal alkaloid biosynthetic enzyme, a steroidal saponin biosynthetic enzyme, or a triterpenoid saponin biosynthetic enzyme, for example but not limited to a CSLG enzyme, have been described in detail above under the header Genetically Modified Cells & Genetically Modified Plants. Similarly, methods for transforming a plant cell with at least one silencing molecule targeted to a nucleic acid sequence encoding a steroidal alkaloid biosynthetic enzyme, a steroidal saponin biosynthetic enzyme, or a triterpenoid saponin biosynthetic enzyme, for example a nucleic acid sequence encoding a CSLG enzyme, have been described in detail above under the header Genetically Modified Cells & Genetically Modified Plants. These methods are incorporated herein in their entirety, wherein a skilled artisan would appreciate that the methods used to produce a genetically modified cell comprised within a genetically modified plant or plant part are in certain embodiments, the same methods used to reduce or increase the content of at least one steroidal alkaloid, steroidal saponin, or triterpenoid saponin in a plant or plant part, especially as the genetically modified plants or plant parts described have an altered content of at least one steroidal alkaloid, steroidal saponin, or triterpenoid saponin, derivative thereof, metabolite thereof, or biosynthetic intermediate thereof.

In some embodiments, when the at least one silencing molecule is targeted to a nucleic acid gene sequence encoding a CSLG enzyme, the nucleic acid sequence encoding said CSLG gene is set forth in any one of SEQ ID NOS: 125, 127, 30, 32, 34, 36 38, 40, 65, 80, 93, 95, 97, 99, 101, 103, or 105; or a homolog thereof having at least 80% identity to and at least 80% coverage of, the nucleic acid sequence set forth in any one of SEQ ID NOS: 125, 127, 30, 32, 34, 36 38, 40, 65, 80, 93, 95, 97, 99, 101, 103, or 105. In some embodiments, when a nucleic acid gene sequence encoding a CSLG enzyme is mutated, the nucleic acid sequence encoding said CSLG gene is set forth in any one of SEQ ID NOS: 125, 127, 30, 32, 34, 36 38, 40, 65, 80, 93, 95, 97, 99, 101, 103, or 105; or a homolog thereof having at least 80% identity to and at least 80% coverage of, the nucleic acid sequence set forth in any one of SEQ ID NOS: 125, 127, 30, 32, 34, 36 38, 40, 65, 80, 93, 95, 97, 99, 101, 103, or 105. In some embodiments, in a method of increasing or a method of decreasing an at least one steroidal alkaloid, steroidal saponin, or triterpenoid saponin, the amino acid sequence of said CSLG enzyme is set forth in any one of SEQ ID NOS: 126, 31, 33, 35, 37, 39, 41, 66, 81, 94, 96, 98, 100, 102 or 104; or a homolog thereof having at least 80% identity to and at least 80% coverage of, the amino acid sequence set forth in any one of SEQ ID NOS: 126, 31, 33, 35, 37, 39, 41, 66, 81, 94, 96, 98, 100, 102 or 104.

In some embodiments, derivatives of steroidal alkaloids, steroidal saponins, or triterpenoid saponins comprise glycosylated derivatives of, respectively, steroidal alkaloids, steroidal saponins, or triterpenoid saponins.

In some embodiments, the mutation comprises a mutation in a non-coding region. In some embodiments, the mutation comprises a mutation in a coding region. Not limiting examples of mutations in non-coding regions are those mutations that increase or decrease the expression of the CSLG gene. In some embodiments, the mutation comprises overexpression of the CSLG gene, wherein the genetically modified plant cell comprises an increase in at least one steroidal alkaloid, steroidal saponin, or triterpenoid saponin. Methods for overexpression are described in detail herein wherein the skilled artisan would appreciate that a constitutive or inducible promoter may be incorporated into the construct comprising the nucleic acid sequence encoding the CSLG gene. In some embodiments, the mutation comprises reduced expression of the CSLG gene, wherein the genetically modified plant cell comprises a decrease in at least one steroidal alkaloid, steroidal saponin, or triterpenoid saponin. Methods for specifically targeting a nucleic acid sequence in order to reduce expression are detailed throughout, wherein the skilled artisan would appreciate that reduction of expression would in certain embodiments, lead to reduction in content of at least one steroidal alkaloid, steroidal saponin, or triterpenoid saponin.

In some embodiments, in a method of increasing the content of a steroidal alkaloid, a derivative thereof, a metabolite thereof, or a biosynthetic intermediate thereof, a steroidal saponin, a derivative thereof, a metabolite thereof, or a biosynthetic intermediate thereof, or a triterpenoid saponin, a derivative thereof, a metabolite thereof, or a biosynthetic intermediate thereof of the gene encoding the CSLG enzyme is increased in the genetically modified plant cell compared to its expression in a corresponding unmodified plant cell; or said encoded CSLG enzyme has increased activity in the genetically modified plant cell compared to its activity in a corresponding unmodified plant cell; or said encoded CSLG enzyme has increased stability in the genetically modified plant cell compared to its stability in a corresponding unmodified plant cell; or any combination thereof. Methods for mutation of nucleic acids encoding genes of the steroidal alkaloid, steroidal saponin, or triterpenoid saponin biosynthetic pathway, for example but not limited to the CSLG gene, are described in detail above and include mutation the coding region for increased stability or activity or a combination thereof of the enzyme, and or mutation the nucleic acid sequence expressing the CSLG gene for over expression. One skilled in the art would appreciate that those methods of mutation could in certain embodiments be used herein as well.

In some embodiments, a method of reducing at least one steroidal alkaloid reduces a steroidal alkaloid comprising a toxin or a bitter tasting compound or a combination thereof, compared to the content of the steroidal alkaloid in a plant or plant part comprising a non-modified plant cell. In some embodiments, a method of reducing at least one steroidal alkaloid eliminates a steroidal alkaloid comprising a toxin or a bitter tasting compound or a combination thereof, compared to the content of the steroidal alkaloid in a plant or plant part comprising a non-modified plant cell. In some embodiments, a method of reducing at least one steroidal alkaloid reduces a steroidal alkaloid, derivative thereof, metabolite thereof, or biosynthetic intermediate thereof, comprising a toxin or a bitter tasting compound or a combination thereof, compared to the content of the steroidal alkaloid, derivative thereof, metabolite thereof, or biosynthetic intermediate thereof, in a plant or plant part comprising a non-modified plant cell. In some embodiments, a method of reducing at least one steroidal alkaloid, derivative thereof, metabolite thereof, or biosynthetic intermediate thereof, eliminates a steroidal alkaloid, derivative thereof, metabolite thereof, or biosynthetic intermediate thereof, comprising a toxin or a bitter tasting compound or a combination thereof, compared to the content of the steroidal alkaloid in a plant or plant part comprising a non-modified plant cell.

In some embodiments, the at least one steroidal alkaloid comprises an esculeoside or a dehydroesculeoside. In some embodiments, the at least one steroidal alkaloid comprises alpha-tomatine, tomatine, dehydrotomatine, alpha-chaconine, alpha-solanine, alpha-solasonine, alpha-solmargine, or any combination thereof. In some embodiments, the reduced at least one steroidal alkaloid comprises an esculeoside or a dehydroesculeoside. In some embodiments, the reduced at least one steroidal alkaloid comprises alpha-tomatine, tomatine, dehydrotomatine, alpha-chaconine, alpha-solanine, alpha-solasonine, alpha-solmargine, or any combination thereof. In some embodiments, the reduced at least one steroidal alkaloid biosynthetic intermediate comprises cholesterol, 22-hydroxycholesterol, 22,26-dihydroxycholesterol, furostanol-type saponin aglycone, furostanol-26-aldehyde, 26 amino-furostanol, tomatidenol, tomatidine, tomatidine galactoside, gamma-tomatine, or beta-1-tomatine, or any combination thereof.

In some embodiments, the reduced at least one steroidal alkaloid comprises eliminating or nearly eliminating an esculeoside or a dehydroesculeoside. In some embodiments, the reduced at least one steroidal alkaloid comprises eliminating or nearly eliminating alpha-tomatine, tomatine, dehydrotomatine, alpha-chaconine, alpha-solanine, alpha-solasonine, alpha-solmargine, or any combination thereof. In some embodiments, the reduced at least one steroidal alkaloid biosynthetic intermediate comprises eliminating or nearly eliminating cholesterol, 22-hydroxycholesterol, 22,26-dihydroxycholesterol, furostanol-type saponin aglycone, furostanol-26-aldehyde, 26 amino-furostanol, tomatidenol, tomatidine, tomatidine galactoside, gamma-tomatine, or beta-1-tomatine, or any combination thereof. In some embodiments, a method of reducing at least one steroidal alkaloid comprises eliminating a combination of at least one steroidal alkaloid and at least one steroidal alkaloid intermediate.

In some embodiments, a method of increasing at least one steroidal alkaloid increases a steroidal alkaloid comprising a toxin or a bitter tasting compound or a combination thereof, compared to the content of the steroidal alkaloid in a plant or plant part comprising a non-modified plant cell. In some embodiments, a method of increasing the content of a steroidal alkaloid, a derivative thereof, a metabolite thereof, or a biosynthetic intermediate thereof increases a steroidal alkaloid comprising an esculeoside or a dehydroesculeoside. In some embodiments, a method of increasing the content of a steroidal alkaloid, a derivative thereof, a metabolite thereof, or a biosynthetic intermediate thereof increases a steroidal alkaloid comprising alpha-tomatine, tomatine, dehydrotomatine, alpha-chaconine, alpha-solanine, alpha-solasonine, alpha-solmargine, or any combination thereof.

In some embodiments, a method of increasing the content of a steroidal alkaloid, a derivative thereof, a metabolite thereof, or a biosynthetic intermediate thereof increases at least one steroidal alkaloid biosynthetic intermediate comprises cholesterol, 22-hydroxycholesterol, 22,26-dihydroxycholesterol, furostanol-type saponin aglycone, furostanol-26-aldehyde, 26-amino-furostanol, tomatidenol, tomatidine, tomatidine galactoside, gamma-tomatine, or beta-1-tomatine, or any combination thereof. In some embodiments, a method of increasing the content of a steroidal alkaloid, a derivative thereof, a metabolite thereof, or a biosynthetic intermediate thereof increases a combination thereof.

In some embodiments, a method of reducing at least one steroidal saponin reduces a steroidal saponin comprising a toxin or a bitter tasting compound or a combination thereof, compared to the content of the steroidal saponin in a plant or plant part comprising a non-modified plant cell. In some embodiments, a method of reducing at least one steroidal saponin eliminates a steroidal saponin comprising a toxin or a bitter tasting compound or a combination thereof, compared to the content of the steroidal saponin in a plant or plant part comprising a non-modified plant cell. In some embodiments, a method of reducing at least one steroidal saponin reduces a steroidal saponin, derivative thereof, metabolite thereof, or biosynthetic intermediate thereof, comprising a toxin or a bitter tasting compound or a combination thereof, compared to the content of the steroidal saponin, derivative thereof, metabolite thereof, or biosynthetic intermediate thereof, in a plant or plant part comprising a non-modified plant cell. In some embodiments, a method of reducing at least one steroidal saponin, derivative thereof, metabolite thereof, or biosynthetic intermediate thereof, eliminates a steroidal saponin, derivative thereof, metabolite thereof, or biosynthetic intermediate thereof, comprising a toxin or a bitter tasting compound or a combination thereof, compared to the content of the steroidal saponin in a plant or plant part comprising a non-modified plant cell.

In some embodiments, the at least one steroidal saponin comprises uttroside B, a tomatoside, or any combination thereof. In some embodiments, the reduced at least one steroidal saponin comprises uttroside B, a tomatoside or any combination thereof. In some embodiments, the reduced at least one steroidal alkaloid biosynthetic intermediate comprises cholesterol, 22-hydroxycholesterol, 22,26-dihydroxycholesterol, furostanol-type saponin aglycone.

In some embodiments, the reduced at least one steroidal saponin comprises eliminating or nearly eliminating uttroside B, a tomatoside or any combination thereof. In some embodiments, the reduced at least one steroidal alkaloid biosynthetic intermediate comprises eliminating or nearly eliminating cholesterol, 22-hydroxycholesterol, 22,26-dihydroxycholesterol, furostanol-type saponin aglycone. In some embodiments, a method of reducing at least one steroidal saponin comprises eliminating a combination of at least one steroidal saponin and at least one steroidal saponin biosynthetic intermediate.

In some embodiments, a method of increasing at least one steroidal saponin increases a steroidal saponin comprising a toxin or a bitter tasting compound or a combination thereof, compared to the content of the steroidal saponin in a plant or plant part comprising a non-modified plant cell. In some embodiments, a method of increasing the content of a steroidal saponin, a derivative thereof, a metabolite thereof, or a biosynthetic intermediate thereof increases a steroidal saponin comprising uttroside B, a tomatoside, or any combination thereof.

In some embodiments, a method of increasing the content of a steroidal saponin, a derivative thereof, a metabolite thereof, or a biosynthetic intermediate thereof increases at least one steroidal saponin biosynthetic intermediate comprising cholesterol, 22-hydroxycholesterol, 22,26-dihydroxycholesterol, furostanol-type saponin aglycone or any combination thereof. In some embodiments, a method of increasing the content of a steroidal saponin, a derivative thereof, a metabolite thereof, or a biosynthetic intermediate thereof increases a combination thereof.

In some embodiments, a method of reducing at least one triterpenoid saponin reduces a triterpenoid saponin comprising a toxin or a bitter tasting compound or a combination thereof, compared to the content of the triterpenoid saponin in a plant or plant part comprising a non-modified plant cell. In some embodiments, a method of reducing at least one triterpenoid saponin eliminates a triterpenoid saponin comprising a toxin or a bitter tasting compound or a combination thereof, compared to the content of the triterpenoid saponin in a plant or plant part comprising a non-modified plant cell. In some embodiments, a method of reducing at least one triterpenoid saponin reduces a triterpenoid saponin, derivative thereof, metabolite thereof, or biosynthetic intermediate thereof, comprising a toxin or a bitter tasting compound or a combination thereof, compared to the content of the triterpenoid saponin, derivative thereof, metabolite thereof, or biosynthetic intermediate thereof, in a plant or plant part comprising a non-modified plant cell. In some embodiments, a method of reducing at least one triterpenoid saponin, derivative thereof, metabolite thereof, or biosynthetic intermediate thereof, eliminates a triterpenoid saponin, derivative thereof, metabolite thereof, or biosynthetic intermediate thereof, comprising a toxin or a bitter tasting compound or a combination thereof, compared to the content of the triterpenoid saponin in a plant or plant part comprising a non-modified plant cell.

In some embodiments, the reduced at least one triterpenoid saponin comprises medicagenic acid 3-O-glucuronide (MA-3-GlcA) (Compound 6), Compound 7, Compound 8, Compound 9, Compound 10, Compound 11, glycyrrhizin (Compound 14), Glycyrrhetinic acid 3-O-monoglucuronide (compound 15), bayogenin (Compound 25), bayogenin-hexA-hex-hex (Compound 31), serjanic acid (Compound 26), serjanic acid-hexA-hex (Compound 32), soyasapogenol A (Compound 29), soyasapogenol B (Compound 30), soyasapogenol A-hexA-hex-pent (Compound 34), soyasaponin VI (Compound 35), soyasaponin I (Compound 36), betavulgaroside IV (Compound 33), hederagenin-3GlcA, gypsogenin-3GlcA, gypsogenic acid-3GlcA, or a QS-21 adjuvant, or any combination thereof. In some embodiments, the reduced triterpenoid saponin intermediate comprises Compound 1, Compound 2, medicagenic acid (Compound 5), oleanolic acid (Compound 3), oleanolic acid-3GlcA, augustic acid (Compound 4), augustic acid-3GlcA, or glycyrrhetinic acid (Compound 13), or any combination thereof. In some embodiments, a method of reducing at least one triterpenoid saponin comprises reducing a combination of at least one triterpenoid saponin and one triterpenoid saponin biosynthetic intermediate.

In some embodiments, the reduced at least one triterpenoid saponin comprises eliminating or nearly eliminating medicagenic acid 3-O-glucuronide (MA-3-GlcA) (Compound 6), Compound 7, Compound 8, Compound 9, Compound 10, Compound 11, glycyrrhizin (Compound 14), Glycyrrhetinic acid 3-O-monoglucuronide (compound 15), bayogenin (Compound 25), bayogenin-hexA-hex-hex (Compound 31), serjanic acid (Compound 26), serjanic acid-hexA-hex (Compound 32), soyasapogenol A (Compound 29), soyasapogenol B (Compound 30), soyasapogenol A-hexA-hex-pent (Compound 34), soyasaponin VI (Compound 35), betavulgaroside IV (Compound 33), hederagenin-3GlcA, gypsogenin-3GlcA, gypsogenic acid-3GlcA, or a QS-21 adjuvant, or any combination thereof. In some embodiments, the reduced triterpenoid saponin intermediate comprises eliminating or nearly eliminating Compound 1, Compound 2, medicagenic acid (Compound 5), oleanolic acid (Compound 3), oleanolic acid-3GlcA, augustic acid (Compound 4), augustic acid-3GlcA, or glycyrrhetinic acid (Compound 13), or any combination thereof. In some embodiments, a method of reducing at least one triterpenoid saponin comprises eliminating a combination of at least one triterpenoid saponin and one triterpenoid saponin biosynthetic intermediate.

In some embodiments, a method of increasing the content of a triterpenoid saponin, a derivative thereof, a metabolite thereof, or a biosynthetic intermediate thereof increases a triterpenoid saponin comprising a sweetener, foaming agent, emulsifier, preservative, anti-carcinogen, hypocholesterolemic agent, anti-inflammatory agent, anti-oxidant, biological adjuvant, anti-microbial agent, insecticidal agent, antifeedant, anti-fungal agent, or any combination thereof. In some embodiments, a method of increasing the content of a triterpenoid saponin, a derivative thereof, a metabolite thereof, or a biosynthetic intermediate thereof increases a triterpenoid saponin comprising medicagenic acid 3-O-glucuronide (MA-3-GlcA) (Compound 6), Compound 7, Compound 8, Compound 9, Compound 10, Compound 11, glycyrrhizin (Compound 14), Glycyrrhetinic acid 3-O-monoglucuronide (compound 15), bayogenin (Compound 25), bayogenin-hexA-hex-hex (Compound 31), serjanic acid (Compound 26), serjanic acid-hexA-hex (Compound 32), soyasapogenol A (Compound 29), soyasapogenol B (Compound 30), soyasapogenol A-hexA-hex-pent (Compound 34), soyasaponin VI (Compound 35), soyasaponin I (Compound 36), betavulgaroside IV (Compound 33), hederagenin-3GlcA, gypsogenin-3GlcA, gypsogenic acid-3GlcA, or a QS-21 adjuvant, or any combination thereof. In some embodiments, a method of increasing the content of a triterpenoid saponin, increases the triterpenoid saponin glycyrrhizin (Compound 14). In some embodiments, a method of increasing the content of a triterpenoid saponin, increases the triterpenoid saponin QS-21 adjuvant.

In some embodiments, a method of increasing the content of a triterpenoid saponin, a derivative thereof, a metabolite thereof, or a biosynthetic intermediate thereof increases a triterpenoid saponin biosynthetic intermediate comprising Compound 1, Compound 2, medicagenic acid (Compound 5), oleanolic acid (Compound 3), oleanolic acid-3GlcA, augustic acid (Compound 4), augustic acid-3GlcA, or glycyrrhetinic acid (Compound 13), or any combination thereof. In some embodiments, a method of increasing the content of a triterpenoid saponin, a derivative thereof, a metabolite thereof, or a biosynthetic intermediate thereof increases a combination thereof.

In some embodiments, a method of increasing the content of at least one steroidal alkaloid, a derivative thereof, a metabolite thereof, or a biosynthetic intermediate thereof, at least one steroidal saponin, a derivative thereof, a metabolite thereof, or a biosynthetic intermediate thereof, at least one triterpenoid saponin, a derivative thereof, a metabolite thereof, or a biosynthetic intermediate thereof, increases the content of the content of at least one steroidal alkaloid, a derivative thereof, a metabolite thereof, or a biosynthetic intermediate thereof, at least one steroidal saponin, a derivative thereof, a metabolite thereof, or a biosynthetic intermediate thereof, at least one triterpenoid saponin, a derivative thereof, a metabolite thereof, or a biosynthetic intermediate thereof, and alters the content of a phytosterol, or a derivative thereof, a metabolite thereof, or a biosynthetic intermediate thereof, a cholesterol, or a derivative thereof, a metabolite thereof, or a biosynthetic intermediate thereof, a phytocholesterol, or a derivative thereof, a metabolite thereof, or a biosynthetic intermediate thereof; a cholestenol, or a derivative thereof, a metabolite thereof, or a biosynthetic intermediate thereof, or a phytocholestenol, or a derivative thereof, a metabolite thereof, or a biosynthetic intermediate thereof.

In some embodiments, a method of increasing the content of at least one steroidal alkaloid, a derivative thereof, a metabolite thereof, or a biosynthetic intermediate thereof, at least one steroidal saponin, a derivative thereof, a metabolite thereof, or a biosynthetic intermediate thereof, at least one triterpenoid saponin, a derivative thereof, a metabolite thereof, or a biosynthetic intermediate thereof, increases the content of the content of at least one steroidal alkaloid, a derivative thereof, a metabolite thereof, or a biosynthetic intermediate thereof, at least one steroidal saponin, a derivative thereof, a metabolite thereof, or a biosynthetic intermediate thereof, at least one triterpenoid saponin, a derivative thereof, a metabolite thereof, or a biosynthetic intermediate thereof, and decreases the content of a phytosterol, or a derivative thereof, a metabolite thereof, or a biosynthetic intermediate thereof, a cholesterol, or a derivative thereof, a metabolite thereof, or a biosynthetic intermediate thereof, a phytocholesterol, or a derivative thereof, a metabolite thereof, or a biosynthetic intermediate thereof, a cholestenol, or a derivative thereof, a metabolite thereof, or a biosynthetic intermediate thereof, or a phytocholestenol, or a derivative thereof, a metabolite thereof, or a biosynthetic intermediate thereof.

In some embodiments, the content of at least one steroidal alkaloid, a derivative thereof, a metabolite thereof, or a biosynthetic intermediate thereof, at least one steroidal saponin, a derivative thereof, a metabolite thereof, or a biosynthetic intermediate thereof, at least one triterpenoid saponin, a derivative thereof, a metabolite thereof, or a biosynthetic intermediate thereof, decreases the content of at least one steroidal alkaloid, a derivative thereof, a metabolite thereof, or a biosynthetic intermediate thereof, at least one steroidal saponin, a derivative thereof, a metabolite thereof, or a biosynthetic intermediate thereof, at least one triterpenoid saponin, a derivative thereof, a metabolite thereof, or a biosynthetic intermediate thereof and an altered content of a phytosterol, or a derivative thereof, a metabolite thereof, or a biosynthetic intermediate thereof, a cholesterol, or a derivative thereof, a metabolite thereof, or a biosynthetic intermediate thereof, a phytocholesterol, or a derivative thereof, a metabolite thereof, or a biosynthetic intermediate thereof; a cholestenol, or a derivative thereof, a metabolite thereof, or a biosynthetic intermediate thereof, or a phytocholestenol, or a derivative thereof, a metabolite thereof, or a biosynthetic intermediate thereof.

In some embodiments, the content of at least one steroidal alkaloid, a derivative thereof, a metabolite thereof, or a biosynthetic intermediate thereof, at least one steroidal saponin, a derivative thereof, a metabolite thereof, or a biosynthetic intermediate thereof, at least one triterpenoid saponin, a derivative thereof, a metabolite thereof, or a biosynthetic intermediate thereof, decreases the content of at least one steroidal alkaloid, a derivative thereof, a metabolite thereof, or a biosynthetic intermediate thereof, at least one steroidal saponin, a derivative thereof, a metabolite thereof, or a biosynthetic intermediate thereof, at least one triterpenoid saponin, a derivative thereof, a metabolite thereof, or a biosynthetic intermediate thereof and an increased content of a phytosterol, or a derivative thereof, a metabolite thereof, or a biosynthetic intermediate thereof, a cholesterol, or a derivative thereof, a metabolite thereof, or a biosynthetic intermediate thereof, a phytocholesterol, or a derivative thereof, a metabolite thereof, or a biosynthetic intermediate thereof, a cholestenol, or a derivative thereof, a metabolite thereof, or a biosynthetic intermediate thereof, or a phytocholestenol, or a derivative thereof, a metabolite thereof, or a biosynthetic intermediate thereof.

In some embodiments in a method of altering (increasing or decreasing) the content of a steroidal alkaloid, a derivative thereof, a metabolite thereof, or a biosynthetic intermediate thereof, a steroidal saponin, a derivative thereof, a metabolite thereof, or a biosynthetic intermediate thereof, or a triterpenoid saponin, a derivative thereof, a metabolite thereof, or a biosynthetic intermediate thereof the method comprises genetically modifying at least one plant cell, wherein in some embodiments said plant cell comprises a leaf cell, a petiole cell, a plant stem or stalk cell, a root cell, a bud cell, a tuber cell, a bean cell, a grain or kernel cell, a fruit cell, a nut cell, a legume cell, a seed or seed cell, a bract cell, a callus cell, and a flower cell.

In some embodiments in a method of altering (increasing or decreasing) the content of a steroidal alkaloid, a derivative thereof, a metabolite thereof, or a biosynthetic intermediate thereof, a steroidal saponin, a derivative thereof, a metabolite thereof, or a biosynthetic intermediate thereof, or a triterpenoid saponin, a derivative thereof, a metabolite thereof, or a biosynthetic intermediate thereof the method comprises genetically modifying at least one plant cell, wherein in some embodiments said plant cell comprises an order, genus, or species recited herein in detail. For example, but not limited to a plant cell comprising a cell from a plant in the Poales order, the Caryophyllales order, the Solanales order, the Fabales order, the Malvales order, the Apiales order, the Brassicales order, the Asparagales order, the Dioscoreales order, or the Liliales order.

In some embodiments of methods altering the content (increasing or decreasing) of a steroidal alkaloid, a steroidal saponin, or a triterpenoid saponin, the plant of the Caryophyllales order is selected from the group of genera consisting of the Spinacia genus, the Chenopodium genus, the Beta genus, and the Rheum genus; or the plant of the Solanales order is selected from the group of genera consisting of the Nicotiana genus, the Solanum genus, and the Capsicum genus; or the plant of the Fabales order is selected from the group of genera consisting of the Pisum sativum genus, the Glycyrrhiza genus, the Medicago genus, the Quillaja genus, the Glycine genus, and the Lotus genus; or the plant of the Apiales order is selected from the group of genera consisting of the Panax genus, Daucus genus, the Apium genus, and the Petroselinum genus, or the plant from the Poales order is selected from the group of genera consisting of the Oryza genus (e.g., Oryza sativa and Oryza glaberrima [rice]), the Hordeum genus (e.g., Hordeum vulgare [barley]), the Avena genus (e.g., Avena sativa [oat], Avena strigosa), and the Triticum genus (e.g., Triticum spelta [spelt]), or the plant from the Brassicales order is selected from the group of genera consisting of the Arabidopsis genus (e.g., Arabidopsis thaliana), the Brassica genus (e.g., Brassica oleracea [cabbages], Brassica juncea [white mustard], Brassica nigra [black mustard], Brassica napus), the Capparis genus (e.g., Capparis spinosa [caper]), and the Carica genus (e.g., Carica papaya [papaya]).

In some embodiments of methods altering the content (increasing or decreasing) of a steroidal alkaloid, a steroidal saponin, or a triterpenoid saponin, the plant of the Solanales order is selected from the group of species consisting of Nicotiana benthamiana, Solanum lycopersicum, Solanum tuberosum, Solanum melongena, Solanum pennellii, Solanum chacoense, Solanum dulcamara, and Capsicum annuum; or the plant of the Fabales order is selected from the group of species consisting of Pisum sativum, Glycyrrhiza uralensis, Medicago sativa, Medicago truncatula, Quillaja saponaria, Glycine max, and Lotus japonicus; or the plant of the Malvales order is selected from the Theobroma genus; or the plant of the Apiales order is selected from the group of species consisting of Panax ginseng, Daucus carota, Apium graveolens, and Petroselinum crispum; or the plant is selected from the species Theobroma cacao.

### Methods of Producing a Steroidal Alkaloid, Steroidal Saponin, or Triterpenoid Saponin in an In Vitro System

In vitro systems are well known in the art. In some embodiments, an in vitro translation system used in the methods for producing a triterpenoid saponin disclosed herein comprises a rabbit reticulocyte lysate, a wheat germ extract, or an E. coli cell-free system. Detailed methods for using a cell free system and known and freely available in the art, for example but not limited to in formation found at: https://www.thermofisher.com/il/en/home/references/ambion-tech-support/large-scale-transcription/general-articles/the-basics-in-vitro-translation.html.

In some embodiments, disclosed herein is a method of producing a steroidal alkaloid or a steroidal saponin in an in vitro system, the method comprising:
(a) combining a nucleic acid sequence or nucleic acid sequences encoding a cytochrome P450 (GAME7) gene, a cytochrome P450 (GAME8) gene, a 2-oxoglutarate-dependent dioxygenase (GAME11) gene, a cytochrome P450 (GAME6) gene, a cytochrome P450 (GAME4) gene, a transaminase (GAME 12) gene, a tomatidine UDP-galactosyltransferase (GAME1/SGT1) gene, a UDP-gycosyltransferase (GAME17) gene, a UDP-gycosyltransferase (GAME18) gene, a UDP-gycosyltransferase/xylosyltransferase (GAME2 SGT3) gene, and a cellulose synthase like G gene (CSLG or GAME1S), wherein said nucleic acid sequence or nucleic acid sequences are optionally comprised in a vector or vectors; and
(b) expressing said genes;
(c) incubating a combination of said expressed enzymes together;
   wherein the product of said incubation comprises at least one steroidal alkaloid or steroidal saponin.

In some embodiments, the above nucleic acid sequence(s) further comprise an ethylene-responsive element binding factor 13 (GAME9) and/or a BHLH-transcription factor.

In some embodiments, disclosed herein is a method of producing a steroidal alkaloid or a steroidal saponin in an in vitro system, the method comprising:
(d) combining a nucleic acid sequence or nucleic acid sequences encoding a cytochrome P450 (GAME7) gene, a cytochrome P450 (GAME8) gene, a 2-oxoglutarate-dependent dioxygenase (GAME11) gene, a cytochrome P450 (GAME6) gene, and a cellulose synthase like G gene (CSLG or GAME1S), wherein said nucleic acid sequence or nucleic acid sequences are optionally comprised in a vector or vectors; and
(e) expressing said genes;
(f) incubating a combination of said expressed enzymes together;
   wherein the product of said incubation comprises at least one steroidal alkaloid or a steroidal saponin, or a combination thereof. See for example but not limited to, the biosynthetic pathways and resultant intermediates and products presented therein in FIGS. 1, 8D, 13, and 14A-14D.

In some embodiments, the biosynthetic GAME genes and/or the CSLG gene are operably linked to a promoter, a transcription termination sequence, or a combination thereof as has been described in detail herein for making constructs. In some embodiments, in a method of producing a steroidal alkaloid or a steroidal saponin, the nucleic acid sequence encoding said CSLG gene is set forth in any one of SEQ ID NOS: 125, 127, 30, 32, 34, 36, 38, 40, 65, 80, 93, 95, 97, 99, 101, 103, or 105; or a homolog thereof having at least 55% identity to and at least 55% coverage of, the nucleic acid sequence set forth in any one of SEQ ID NOS: 125, 127, 30, 32, 34, 36, 38, 40, 65, 80, 93, 95, 97, 99, 101. In some embodiments, in a method of producing a steroidal alkaloid or a steroidal saponin, the nucleic acid sequence encoding said CSLG gene is set forth in any one of SEQ ID NOS: 125, 127, 30, 32, 34, 36, 38, 40, 65, 80, 93, 95, 97, 99, 101, 103, or 105; or a homolog thereof having at least 55% identity to and at least 80% coverage of, the nucleic acid sequence set forth in any one of SEQ ID NOS: 125, 127, 30, 32, 34, 36, 38, 40, 65, 80, 93, 95, 97, 99, 101.

In some embodiments, the biosynthetic GAME genes and/or the CSLG gene are operably linked to a promoter, a transcription termination sequence, or a combination thereof as has been described in detail herein for making constructs. In some embodiments, in a method of producing a steroidal alkaloid or a steroidal saponin, the nucleic acid sequence encoding said CSLG gene is set forth in any one of SEQ ID NOS: 125, 127, 30, 32, 34, 36, 38, 40, 65, 80, 93, 95, 97, 99, 101, 103, or 105; or a homolog thereof having at least 80% identity to and at least 80% coverage of, the nucleic acid sequence set forth in any one of SEQ ID NOS: 125, 127, 30, 32, 34, 36, 38, 40, 65, 80, 93, 95, 97, 99, 101.

In certain embodiments, an in vitro method of producing a steroidal alkaloid comprises producing a steroidal alkaloid selected from a esculeoside or a dehydroesculeoside. In certain embodiments, an in vitro method of producing a steroidal alkaloid comprises producing a steroidal alkaloid selected from alpha-tomatine, tomatine, alpha-chaconine, alpha-solanine, alpha-solasonine, alpha-solmargine, or any combination thereof.

In certain embodiments, an in vitro method of producing a steroidal saponin comprises producing a steroidal saponin selected from uttroside B, a tomatoside or any combination thereof.

In some embodiments, disclosed herein is a method of producing a triterpenoid saponin in an in vitro system, the method comprising:
(g) combining a nucleic acid sequence or nucleic acid sequences encoding a saponin beta-amyrin synthase gene (SOAP1), a cytochrome P450 gene (SOAP2), a cytochrome P450 gene expressing a C-2 hydroxylase (SOAP3), a cytochrome P450 gene expressing a C-23 oxidase (SOAP4), a glycosyl transferase gene expressing a UDP-glycosyltransferase or a fucocyl transferase (SOAP6), a glycosyl transferase gene expressing a UDP-glycosyltransferase (SOAP7), a glycosyl transferase gene expressing a UDP-glycosyltransferase (SOAP8); a glycosyl transferase gene expressing a UDP-glycosyltransferase or a xylosyl transferase (SOAP9), an acyltransferase gene (SOAP10), and a cellulose synthase like G gene (CSLG), wherein said nucleic acid sequence or nucleic acid sequences are optionally comprised in a vector or vectors; and
(h) expressing said genes;
(i) incubating a combination of said expressed enzymes together;
   wherein the product of said incubation comprises at least one triterpenoid saponin.

In some embodiments, the biosynthetic SOAP genes and/or the CSLG gene are operably linked to a promoter, a transcription termination sequence, or a combination thereof as has been described in detail herein for making constructs. In some embodiments, in a method of producing a triterpenoid saponin the nucleic acid sequence encoding said CSLG gene is set forth in any one of SEQ ID NOS: 125, 127, 30, 32, 34, 36, 38, 40, 65, 80, 93, 95, 97, 99, 101, 103, or 105; or a homolog thereof having at least 55% identity to and at least 55% coverage of, the nucleic acid sequence set forth in any one of SEQ ID NOS: 125, 127, 30, 32, 34, 36, 38, 40, 65, 80, 93, 95, 97, 99, 101. In some embodiments, in a method of producing a triterpenoid saponin the nucleic acid sequence encoding said CSLG gene is set forth in any one of SEQ ID NOS: 125, 127, 30, 32, 34, 36, 38, 40, 65, 80, 93, 95, 97, 99, 101, 103, or 105; or a homolog thereof having at least 55% identity to and at least 80% coverage of, the nucleic acid sequence set forth in any one of SEQ ID NOS: 125, 127, 30, 32, 34, 36, 38, 40, 65, 80, 93, 95, 97, 99, 101.

In some embodiments, the biosynthetic SOAP genes and/or the CSLG gene are operably linked to a promoter, a transcription termination sequence, or a combination thereof as has been described in detail herein for making constructs. In some embodiments, in a method of producing a triterpenoid saponin the nucleic acid sequence encoding said CSLG gene is set forth in any one of SEQ ID NOS: 125, 127, 30, 32, 34, 36, 38, 40, 65, 80, 93, 95, 97, 99, 101, 103, or 105; or a homolog thereof having at least 80% identity to and at least 80% coverage of, the nucleic acid sequence set forth in any one of SEQ ID NOS: 125, 127, 30, 32, 34, 36, 38, 40, 65, 80, 93, 95, 97, 99, 101.

In some embodiments, the nucleic acid gene sequence or sequences encoding the other triterpenoid saponin biosynthetic enzymes are set forth as follows:
(a) the nucleic acid sequence encoding said β-amyrin synthase gene is set forth in SEQ ID NO: 45; or a homolog thereof having at least 80% identity to and at least 80% coverage of, the nucleic acid sequence set forth in SEQ ID NO: 45; and
(b) the nucleic acid sequence or sequences encoding said cytochrome P450 genes, are set forth in any one of SEQ ID NO: 46, 51, or 53; or a homolog thereof having at least 80% identity to and at least 80% coverage of, the nucleic acid sequence set forth in any one of SEQ ID NO: 46, 51, or 53; and
(c) the nucleic acid sequence or sequences encoding said glycosyl transferase genes, are set forth in any one of SEQ ID NO: 55, 57, 59, or 61; or a homolog thereof having at least 80% identity to and at least 80% coverage of, the nucleic acid sequence set forth in any one of SEQ ID NO: 55, 57, 59, or 61; and
(d) the nucleic acid sequence encoding said acyltransferase is set forth in SEQ ID NO: 63; or a homolog thereof having at least 80% identity to and at least 80% coverage of, the nucleic acid sequence set forth in SEQ ID NO: 63.

In certain embodiments, an in vitro method of producing a triterpenoid saponin comprises producing a triterpenoid saponin selected from a sweetener, foaming agent, emulsifier, preservative, anti-carcinogen, hypocholesterolemic agent, anti-inflammatory agent, anti-oxidant, biological adjuvant, anti-microbial agent, insecticidal agent, antifeedant, anti-fungal agent, or any combination thereof.

In certain embodiments, an in vitro method of producing a triterpenoid saponin comprises producing a triterpenoid saponin selected from medicagenic acid 3-O-glucuronide (MA-3-GlcA) (Compound 6), Compound 7, Compound 8, Compound 9, Compound 10, Compound 11, glycyrrhizin (Compound 14), Glycyrrhetinic acid 3-O-monoglucuronide (compound 15), bayogenin (Compound 25), bayogenin-hexA-hex-hex (Compound 31), serjanic acid (Compound 26), serjanic acid-hexA-hex (Compound 32), soyasapogenol A (Compound 29), soyasapogenol B (Compound 30), soyasapogenol A-hexA-hex-pent (Compound 34), soyasaponin VI (Compound 35), soyasaponin I (Compound 36), betavulgaroside IV (Compound 33), hederagenin-3GlcA, gypsogenin-3GlcA, gypsogenic acid-3GlcA, or a QS-21 adjuvant, or any combination thereof.

As used in the specification and claims, the singular form “a”, “an” and “the” include plural references unless the context clearly dictates otherwise. For example, the term “a molecule” also includes a plurality of molecules.

Throughout this application, various embodiments may be presented in a range format. It should be understood that the description in range format is merely for convenience and brevity and should not be construed as an inflexible limitation. Accordingly, the description of a range should be considered to have specifically disclosed all the possible sub ranges as well as individual numerical values within that range. For example, description of a range such as from 1 to 6 should be considered to have specifically disclosed sub ranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals there between.

Further, one skilled in the art would appreciate that the term “comprising” used throughout is intended to mean that the genetically modified or gene edited plants disclosed herein, and methods of altering expression of genes, and altering production of SA and/or SGA within these genetically modified or gene edited plants includes the recited elements, but not excluding others which may be optional. “Consisting of” shall thus mean excluding more than traces of other elements. The skilled artisan would appreciate that while, in some embodiments the term “comprising” is used, such a term may be replaced by the term “consisting of”, wherein such a replacement would narrow the scope of inclusion of elements not specifically recited.

The following examples are presented in order to more fully illustrate some embodiments of the invention. They should, in no way be construed, however, as limiting the broad scope of the invention. They should, in no way be construed, however, as limiting the broad scope of the genetically modified cells and plants disclosed herein or the uses of said cells and plants to produce steroidal alkaloids, steroidal saponins, or triterpenoid saponins; or methods for increasing or decreasing the content of a steroidal alkaloid, steroidal saponin, or triterpenoid saponin in a plant or plant part. One skilled in the art can readily devise many variations and modifications of the principles disclosed herein without departing from the scope disclosed herein.

### EXAMPLES

### Example 1: Materials and Methods for Examples 2-13

### Plant Material, Treatments, and Generation of Transgenic Plants

Tomato (Solanum lycopersicum; cv. Micro Tom) and potato (Solanum tuberosum; cultivar Desiree) plants were collected as described previously (Itkin et al., 2001, supra). In potato, when the green parts started to dry, mature tubers (Stage 3) were collected, washed of soil, dried and kept at 4° C., at complete darkness.

The GAME9-silenced (RNAi) and overexpression (OX) constructs were created by introducing the corresponding GAME9 DNA fragments to pK7GWIWG2(II) and pJCV52 binary vectors, respectively. Transgenic lines for silencing and overexpression of GAME9 in tomato and potato were generated and tissue extracts were prepared and analyzed according to Itkin et al. (2011, supra).

Table 3 below describes the oligonucleotides used for generation of the constructs described herein. The GAME4-silencing (RNAi; GAME4i), GAME4 overexpressing (GAME4oe) and GAME8-silencing constructs were generated as described previously (Itkin et al., 2001, supra; WO 2012/095843).

TABLE 3
Oligonucleotides used for construct production

| | Sequence 5' to 3'/ | SEQ |
|---|---|---|
| Name | Description | ID NO. |
| Sl07g043420 | AAAAAgaattcCGGATCTTCTCTCGAACTGGTCAA | 1 |
| EcoRI Fw | To prepare GAME11 virus-induced gene silencing | |
| | (VIGS) construct | |
| Sl07g043420 | AAAAAgaattcCACTTTCATTGCTTCATCCATTAGATCT | 2 |
| EcoRI Rv | To prepare GAME11 VIGS construct | |
| Sl07g043500 | AAAAAgaattcCTTAGCTTATGGCCACATCACACCTT | 3 |
| EcoRI Fw | To prepare GAME18 VIGS construct | |
| Sl07g043500 | AAAAAgaattcACTCAAGATTTGGTGAAGCTGTGGTT | 4 |
| EcoRI Rv | To prepare GAME18 VIGS construct | |
| G8-Forward | AAAAAGGCGCGCCAATCATAGAGAAGAAAGAAGACG | 5 |
| (AscI) | To construct RNAi of GAME8 | |
| G8-Reverse | AAAAAGCGGCCGCACTCCTGCAGGAATTGTCATTTCTC | 6 |
| (NotI) | To construct RNAi of GAME8 | |
| GAME9 RNAi | aaaaaGCGGCCGCATGAGTATTGTAATTGATGATGATGAA | 7 |
| NotI Fw | ATC | |
| | To construct RNAi of GAME9 | |
| GAME9 RNAi | aaaaGGCGCGCCCACACGCCACAGATGGTTCTT | 8 |
| AscI Rv | To construct RNAi of GAME9 | |
| GAME9-Tom | GGGGACAAGTTTGTACAAAAAAGCAGGCTATGAGTATT | 9 |
| GW Fw | GTAATTGATGATGATGAAATC | |
| | To pick up the gene from cDNA for overexpression | |
| | (good for tomato) | |
| GAME9-Tom | GGGGACCACTTTGTACAAGAAAGCTGGGTTCATACTAC | 10 |
| GW Rv | CTTCTGTCCTAAGCCT | |
| | To pick up the gene from cDNA for overexpression | |
| | (good for tomato) | |
| GAME9-Pot | GGGGACAAGTTTGTACAAAAAAGCAGGCTATGAATATT | 11 |
| GW Fw | GCAATTGATGATGATGA | |
| | To pick up the gene from cDNA for overexpression | |
| | (good for potato) | |
| GAME9-Pot | GGGGACCACTTTGTACAAGAAAGCTGGGTTCATTTGTAT | 12 |
| GW Rv | CAACATTTGTAAATTCACAC | |
| | To pick up the gene from cDNA for overexpression | |
| | (good for potato) | |

### Co-Expression Analysis

The tomato GAME1 (Solyc07g043490) and its potato ortholog SGT1 (PGSC003DMG400011749) were used as ‘baits’ in the co-expression analysis, resulting in lists (sorted in descending order by r-value >0.8) of co-expressed genes (for each ‘bait’ separately). Two homologous genes were subsequently identified (Solyc12g006460 and PGSC0003DMG400024274 in tomato and potato, respectively), which were highly correlated with the “bait” genes (r-value >0.9 in both species). Those genes were identified as GLYCOALKALOID METABOLISM 4 (GAME4, WO 2012/095843). The GAME4 genes were further added as ‘baits’ to the previous (GAME1) co-expression analysis. The co-expression lists for GAME1 (SGT1) and GAME4 in both species were used to construct co-expression correlation network. The analysis was performed as follows: tomato RNAseq transcriptome data from different tissues and organs (flesh, peel, seeds, roots, leaves, buds, flowers, pollen) and developmental stages (25 experiments in total) (Itkin et al., 2011, ibid) and potato RNAseq transcriptome data from different tissues and organs (40 experiments in total) (US 2012/0159676), were used. First, an R script was used to perform co-expression analysis (for each species) and the list of co-expressed genes was constructed as a FASTA file, using a Perl script. Finally, BLASTall tools (Camacho C. et al., 2009. BMC Bioinform 10:421) were used to find shared homologs between the two species. The tblastx criteria for homolog similarity were set to p-value >0.05, minimum 25 nucleotides, and at least 60 percent similarity as an overall identity for each gene. The co-expression network was visualized with the Cytoscape program (Shannon P. et al., 2003. Genome Res. 13:2498-2504).

### Phylogenetic Analysis

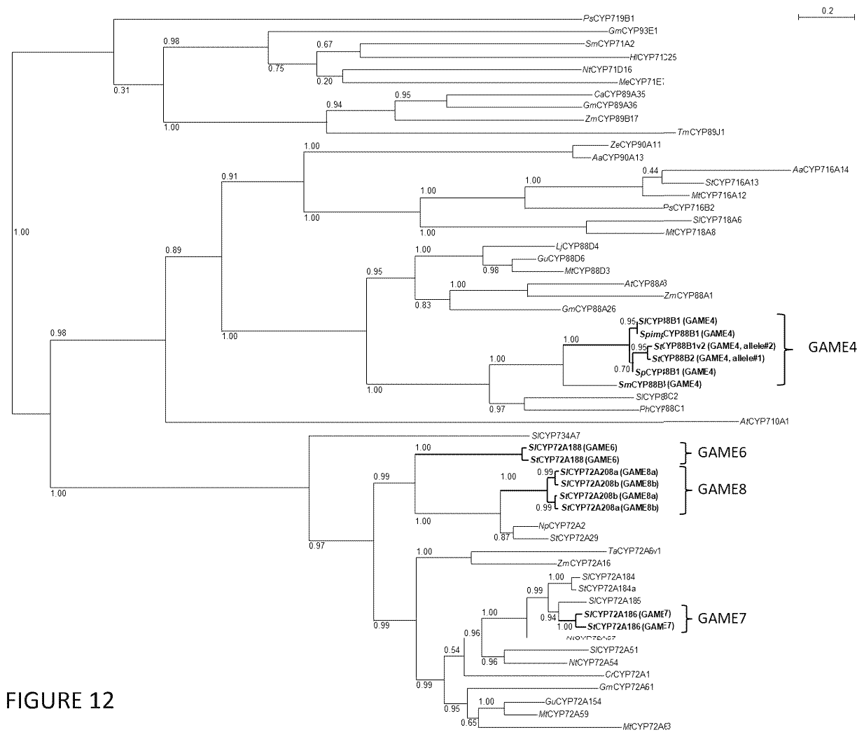

The protein sequences were aligned using the Muscle algorithm and the phylogenetic tree was analyzed and visualized by the SeaView v4.3.5 program using the maximum likelihood method by PhyML 3.0 (Expósito-Rodríguez M et al., 2008. BMC Plant Biol. 8:131) with the following settings: model—LG; The approximate likelihood ratio test (aLRT) Shimodaira-Hasegawa-like (SH-like) procedure was used as a statistical test to calculate branch support (branch support—aLRT (SH-like)); invariable sites—optimized; across site rate variation—optimized; tree searching operations—best for NNI & SPR; starting tree—BioNJ, optimize tree topology. The numbers on the branches indicate the fraction of bootstrap iterations supporting each node. The accession numbers of the proteins used for the preparation of this tree and the organism names are listed in Table 4 hereinbelow; the tree is presented in FIG. 12.

TABLE 4
Accession numbers of the sequences used for the construction of the phylogenetic tree

| Name as appears in FIG. 12 | Latin and common name | Accession number |
|---|---|---|
| GuCYP88D6 | Glycyrrhiza uralensis | BAG68929.1 |
| LjCYP88D4 | Lotus japonicus | BAG68927.1 |
| MtCYP88D3 | Medicago truncatula | BAG68926.1 |
| CmCYP88A2 | Cucurbita maxima | AF212991 |
| AtCYP88A3 | Arabidopsis thaliana | AAB71462.1 |
| PsCYP88A7 | Pisum sativum | AAO23064.1 |
| ZmCYP88A1 | Zea mays | NP_001105586.1 |
| GmCYP88A26 | Glycine max | XP_003516638.1 |
| CaCYP89A35 | Capsicum annuum | DQ114394 |
| GmCYP89A36 | Glycine max | DQ340245 |
| ZmCYP89B17 | Zea mays | CO465851.1 |
| TmCYP89J1 | Triticum monococcum | AY914081 |
| SlCYP88B1 (GAME4) | Solanum lycopersicum | Solyc12g006460.1.1 |
| SpimpCYP88B1 (GAME4) | Solanum pimpinellifolium | contig 6356779 |
| SpCYP88B1 (GAME4) | Solanum pinelii | AW618484.1, BG135958.1 |
| StCYP88B2 (GAME4) | Solanum tuberosum group Phureja | PGSC0003DMP400041994 |
| StCYP88B1v2 (GAME4) | Solanum tuberosum group Tuberosum | PGSC0003DMP400041994 |
| SlCYP88C2 | Solanum lycopersicum | Solyc10g007860.2.1 |
| SmCYP88B3 (GAME4) | Solanum melongena | FS071104, FS071103 |
| OsCYP90A3 | Oryza sativa | AC123526.1 |
| SlCYP90A5 | Solanum lycopersicum | Solyc06g051750.2.1 |
| ScCYP90A8 | Citrus sinensis | DQ001728.1 |
| ZeCYP90A11 | Zinnia elegans | BAE16977.1 |
| PhCYP88C1 | Petunia hybrida | AAZ39647.1 |
| AaCYP90A13 | Artemisia annua | ABC94481.1 |
| AtCYP710A1 | Arabidopsis thaliana | AAC26690.1 |
| SmCYP71A2 | Solanum melongena | X71654.1 |
| GmCYP93E1 | Glycine max | AB231332 |
| HICYP71C25 | Hordeum lechleri | AY462228 |
| NtCYP71D16 | Nicotiana tabacum | AF166332 |
| MeCYP71E7 | Manihot esculenta | AY217351 |
| TaCYP71F1 | Triticum aestivum | AB036772 |
| AoCYP71J1 | Asparagus officinalis | AB052131 |
| MaCYP71N1v2 | Musa acuminata | AY062167 |
| TaCYP72A6v1 | Triticum aestivum | AF123604 |
| ZmCYP72A16 | Zea mays | AF465265 |
| LeCYP72A51 | Solanum lycopersicum | Solyc10g051020.1.1 |
| GmCYP72A61 | Glycine max | DQ340241 |
| MtCYP716A12 | Medicago truncatula | ABC59076.1 |
| StCYP716A13 | Solanum tuberosum | PGSC0003DMP400013378 |
| AaCYP716A14 | Artemisia annua | DQ363134 |
| PsCYP716B2 | Picea sitchensis | AY779543 |
| SlCYP718A6 | Solanum lycopersicum | Solyc07g055970.1.1 |
| MtCYP718A8 | Medicago truncatula | XP_003617455.1 |
| PsCYP719B1 | Papaver somniferum | EF451150 |
| StCYP72A186 (GAME7) | Solanum tuberosum | PGSC0003DMG402012386 |
| SlCYP72A186 (GAME7) | Solanum lycopersicum | Solyc07g062520 |
| SlCYP72A188 (GAME6) | Solanum lycopersicum | Solyc07g043460 |
| StCYP72A188 (GAME6) | Solanum tuberosum | PGSC0003DMG400011750 |
| GuCYP72A154 | Glycyrrhiza uralensis | BAL45206.1 |
| MtCYP72A59 | Medicago truncatula | ABC59078.1 |
| NtCYP72A57 | Nicotiana tabacum | ABC69414.1 |
| NtCYP72A54 | Nicotiana tabacum | ABC69417.1 |
| CrCYP72A1 | Catharanthus roseus | gi461812 |
| MtCYP72A63 | Medicago truncatula | gi371940452 |
| NpCYP72A2 | Nicotiana plumbaginifolia | AAB05376.3 |
| SlCYP734A7 | Solanum lycopersicum | Solyc03g120060.1.1 |
| StCYP72A29 | Solanum tuberosum | BAB86912.1 |
| StSYP72a56 | Solanum tuberosum | PGSC0003DMG400017325 |
| StCYP72A208 (GAME8a) | Solanum tuberosum | PGSC0003DMG400026594 |
| StCYP72A208 (GAME8b) | Solanum tuberosum | PGSC0003DMG400026586 |
| SlCYP72A208 (GAME8a) | Solanum lycopersicum | TC243022 |
| SlCYP72A208 (GAME8b) | Solanum lycopersicum | SGN-U578058 |

### Metabolite Analysis

Preparation of plant tissue extracts and profiling of semi-polar compounds (including steroidal alkaloids and steroidal saponins) by UPLC-qTOF-MS and phytosterol content of the tomato leaves were carried out as described previously (Itkin et al., 2011, supra).

### Quantitative Real-Time PCR Assays

RNA was isolated and Quantitative Real-Time PCR was performed as described previously (Itkin et al., 2011, supra). In addition, the TIP41 gene (23) was used as an endogenous control for the potato samples. Oligonucleotides are listed in Table 3 hereinabove.

### Production of Recombinant Enzyme

GAME2, GAME17 and GAME18 were amplified from cDNA and subcloned into pACYCDUET-1 using BamH I and Pst I (GAME2, GAME18) or BamHI and XhoI (GAME17) restriction sites, and the insert was verified by sequencing. The resulting plasmids, pAC-GAME2/17/18 were transformed to E. coli BL21 DE3. For expression of the GAME enzymes, fresh overnight cultures were diluted 1:100 in 25 ml 2×YT medium with 30 μg/ml chloramphenicol and incubated at 37° C. and 250 rpm until an A_{600 nm} of 0.4 was reached. Subsequently, IPTG was added to a concentration of 0.5 mM, and the incubation was continued overnight at 18° C. and 250 rpm. The next day, cells were harvested by centrifugation, and the pellet resuspended in 2 ml of 50 mM Tris HCl pH=7.0, 15% glycerol, 0.1 mM EDTA and 5 mM β-mercaptoethanol. After breaking the cells by sonication, insoluble material was removed by centrifugation, and the soluble fractions were used for characterization of the enzymes. Proteins were stored at −20° C. until further analysis.

### Preparation of Substrates

For hydrolysis, 35 mg of α-tomatine was solved in 3 ml of 1N HCl, and was incubated for 15 min. at 100° C. Subsequently, the solution was put on ice, and NH₃ was added until the pH of the solution was 9.0. The solution was extracted with 4 ml water-saturated butanol. The butanol phase was evaporated to dryness under vacuum, the residual pellet solved in 1 ml methanol and stored at −20° C. until further use. The degradation products of α-tomatine were separated on a Luna 5 μm C18(2) 100 Å, LC Column 150×21.2 mm (Phenomenex, USA), using an isocratic elution with 25% acetonitrile in water and 0.1% formic acid. Compounds were detected using a 3100 Mass Detector (Waters), and collected. Fractions were freeze-dried, and purity of compounds was verified by LC-MS. For identification of products, liquid chromatography, coupled to quadrupole time-of-flight mass spectrometry (LC-QTOF-MS) was performed using a Waters Alliance 2795 HPLC connected to a Waters 2996 PDA detector and subsequently a QTOF Ultima V4.00.00 mass spectrometer (Waters, MS Technologies, UK) operated in positive ionization mode. The column used was an analytical Luna 3 μm C18 (2) 100 Å; 150×2.0 mm (Phenomenex, USA) attached to a C18 pre-column (2.0×4 mm; AJO-4286; Phenomenex, USA). Degassed eluent A [ultra-pure water:formic acid (1000:1, v/v)] and eluent B [acetonitrile:formic acid (1000:1, v/v)] were used with flow rate of 0.19 ml/min. The gradient started at 5% B and increased linearly to 75% B in 45 min., after which the column was washed and equilibrated for 15 min. before the next injection. The injection volume was 5 μl. This procedure yielded several milligrams of pure y-tomatine (tomatidine-galactoside-glucoside, T-Gal-Glu) and β1-tomatine (tomatidine-galactoside-diglucoside. T-Gal-Glu-Glu). Tomatidine galactoside (T-Gal) could not be purified in this way due to strong contamination with T-Gal-Glu. Therefore 5 mg tomatidine was incubated with GAME1 and UDP-galactose in 1 ml reaction mix, as described previously (Itkin et al., 2011, supra). T-Gal was purified from UDP-galactose by solid phase extraction. Waters OASIS HLB 3 cc columns (Waters Corp., Milford, MA) was conditioned with 6 mL 100% methanol followed by rinsing with 4 mL ultra-pure water. The reaction, supplemented with 10% methanol, was loaded and the cartridge was subsequently washed with 4 mL ultra-pure water. Compounds were eluted with 1 mL 75% methanol in ultra-pure water (v:v), and 0.4 mL 100% methanol. The solvent was removed from the combined eluate using a speed vacuum concentrator until a totally dry-pellet was obtained.

### Enzyme Assays

The substrates T-Gal, β1- and γ-tomatine were dissolved to 1 mM in 50% DMSO. Enzyme assays were carried out in 50 mM Tris HCl pH=7.0 containing 5 mM β-mercaptoethanol using 5 μg/ml enzyme, 8 mM UDP-xylose and 0.02 mM substrate in a final reaction volume of 100 pl. After 2 h. of incubation under agitation at 37° C., reactions were stopped by addition of 300 μl methanol and 0.1% formic acid, and followed by brief vortexing and sonication for 15 min. Subsequently, the extracts were centrifuged for 5 min. at 13,000 rpm and filtered through 0.45 m filters (Minisart SRP4, Biotech GmbH, Germany), and analyzed by LC-MS (see above). The amount of product was measured by the peak surface area in the LC-MS chromatogram, and compared to a control incubation in which an enzyme preparation of an E. coli harboring an empty pACYCDUET-1. Masses used for detection were α-tomatine (C50H83NO21; m/z=1034.55 ([M+H]+)), β1-tomatine T-Gal-Glu-Glu (C45H75NO17; m/z=902.51 ([M+H])), β2-tomatine (C44H73NO16; m/z=872.50 ([M+H]+)), γ-tomatine T-Gal-Glu (C39H65NO12; m/z=740.46 ([M+H])), and T-Gal (C33H55NO7; m/z=578.41 ([M+H])).

### Virus Induced Gene Silencing (VIGS) Experiments

Vectors containing fragments of GAME genes were constructed and VIGS experiments were conducted as described previously (Orzaea D et al., 2009. Plant Physiol. 150:1122-1134; Li R et al., 2006 J. Mass Spec. 41:1-22). Plants infected with Agrobacterium, containing empty vector and helper vector pTRV1, were used as control. Oligonucleotides used to prepare the pTRV2_DR_GW vectors are listed in Table 3 hereinabove.

### Genome Sequence Analysis of the Wild Tomato Species

Partial genomic data obtained by re-sequencing (Dr. Arnaud G. Bovy, unpublished data) of three tomato wild species genomes (i.e. Solanum pennellii, S. pimpinellifolium and S. chmielewskii) were analyzed for the presence or absence of sequences (contigs) that align to the SGAs biosynthesis gene clusters on tomato chromosomes 7 and 12. The TopHat toolkit (Trapnell C. 2012. Nat. Protoc. 7:562-578) was used for mapping reads of the wild species to the tomato genome (ITAG 2.4), as a reference genome. The mapped reads were visualized with the IGV genome browser (Robinson J T et al., 2011. Nat. Biotechnol. 29:24-26). In order to assemble and align the sequence of the contigs from the three wild species to the gene clusters on to the existing cultivated tomato sequences of chromosomes 7 and 12, a combination of the CLC workbench, CAP3 BWA and SAMtools software packages and an in-house Perl script were used.

### Example 2: Genes Associated with SGA Biosynthesis

To discover genes associated with SGA biosynthesis, a co-expression analysis using transcriptome data from tomato and potato plants was performed. Coexpression with GAME1/SGT1 (chromosome 7) and GAME4 (chromosome 12) as “baits” in either potato or tomato are presented in a form of a heatmap in Tables 5-8 herein below. Genes that are highly co-expressed with either GAME1/SGT1 (chromosome 7) or GAME4 (chromosome 12) are depicted with a large font and bold.

TABLE 5
Accession numbers, putative protein and
co-expression r-values-tomato, chromosome 7

| | | r-value of |
|---|---|---|
| | | correlation with |
| | | tomato GAME1 |
| Gene name | Putative protein | expression |
| Solyc07g043310 | Aminotransferase | −0.26 |
| Solyc07g043320 | Unknown Protein | 0.12 |
| Solyc07g043330 | GRAS family transcription factor | 0.72 |
| Solyc07g043340 | Unknown Protein | |
| Solyc07g043350 | Unknown Protein | |
| Solyc07g043360 | 60S ribosomal protein L27 | 0.10 |
| Solyc07g043370 | Transposase | |
| Solyc07g043380 | Unknown Protein | |
| Solyc07g043390 | Cellulose synthase family protein | 0.92 |
| | (GAME15) | |
| Solyc07g043400 | Unknown Protein | |
| Solyc07g043410 | UDP-xylose xylosyltransferase | |
| | (GAME2) | |
| Solyc07g043420 | 2-oxoglutarate-dependent | 0.79 |
| | dioxy genase | |
| Solyc07g043430 | Gag-Pol polyprotein | |
| Solyc07g043440 | Glucosyltransferase-like protein | |
| Solyc07g043450 | Zeatin O-glucosyltransferase | |
| Solyc07g043460 | Cytochrome P450 (GAME 6) | 0.91 |
| Solyc07g043470 | Unknown Protein | |
| Solyc07g043480 | UDP-glucose glucosyltransferase | 0.88 |
| Solyc07g043490 | UDP-glucosyltransferase family | 1.00 |
| | 1 protein (GAME1) | |
| Solyc07g043500 | UDP-glucosyltransferase | 0.95 |
| Solyc07g043510 | Cysteine-type peptidase | −0.24 |
| Solyc07g043520 | transposase | |
| Solyc07g043530 | Unknown Protein | |
| Solyc07g043540 | Unknown Protein | |
| Solyc07g043550 | UDP-arabinose 4-epimerase | 0.70 |
| Solyc07g043560 | Heat shock protein 4 | 0.24 |
| Solyc07g043570 | Aldo/keto reductase family | −0.09 |
| | protein | |
| Solyc07g043580 | BHLH transcription factor | 0.43 |
| Solyc07g043590 | Amine oxidase family protein | 0.03 |
| Solyc07g043600 | Pentatricopeptide repeat- | 0.43 |
| | containing protein | |
| Solyc07g043610 | Auxin response factor 6 | |
| Solyc07g043620 | Auxin response factor 6-1 | 0.65 |
| Solyc07g043630 | Acyl-CoA synthetase/AMP-acid | |
| | ligase II | |
| Solyc07g043640 | Acyl-CoA synthetase/AMP-acid | |
| | ligase II | |
| Solyc07g043650 | AMP-dependent synthetase and | |
| | ligase | |
| Solyc07g043660 | Acyl-CoA synthetase/AMP-acid | −0.16 |
| | ligase II | |
| Solyc07g043670 | Hydroxycinnamoyl CoA quinate | |
| | transferase 2 | |
| Solyc07g043680 | Enoyl-CoA-hydratase | |
| Solyc07g043690 | Enoy1-CoA-hydratase | |
| Solyc07g043700 | Acyltransferase | |

TABLE 6
Accession numbers, putative protein and co-expression
r-values-potato, chromosome 7

| | | r-value of |
|---|---|---|
| Gene name | Putative protein | correlation with potato SGT1 expression |
| PGSC0003DMG400011754 | Gamma aminobutyrate | −0.31 |
| | transaminase | |
| PGSC0003DMG400011753 | Uro-adherence factor A | −0.40 |
| PGSC0003DMG400011742 | DELLA protein RGA | 0.15 |
| PGSC0003DMG400011741 | 60S ribosomal protein L27 | 0.43 |
| PGSC0003DMG400039612 | Conserved gene of unknown | |
| | function | |
| PGSC0003DMG400011752 | Cellulose synthase (GAME15) | 0.90 |
| PGSC0003DMG400011740 | beta-solanine | 0.90 |
| | rhamnosyltransferase (SGT3) | |
| PGSC0003DMG400011751 | 2-oxoglutarate-dependent | 0.87 |
| | dioxygenase | |
| PGSC0003DMG400011750 | Cytochrome P-450 (GAME 6) | 0.92 |
| PGSC0003DMG400044993 | Unknown Protein | |
| PGSC0003DMG400011749 | solanidine galactosyltransferase | 1.00 |
| | (SGT1) | |
| PGSC0003DMG402015928 | OTU-like cysteine protease | −0.24 |
| | family protein | |
| PGSC0003DMG401015928 | Conserved protein of unknown | −0.25 |
| | function | |
| PGSC0003DMG400015927 | UDP-arabinose 4-epimerase 1 | −0.21 |
| PGSC0003DMG400015920 | Heat shock 70 kDa protein | −0.17 |
| PGSC0003DMG402015926 | Aldo/keto reductase | −0.05 |
| PGSC0003DMG401015926 | Isoform 2 of Transcription | −0.33 |
| | factor PIF5 | |
| PGSC0003DMG400015925 | Amine oxidase | 0.11 |
| PGSC0003DMG400015924 | Pentatricopeptide repeat- | 0.32 |
| | containing protein | |
| PGSC0003DMG400015919 | ARF8 | 0.07 |
| PGSC0003DMG400036440 | AMP dependent ligase | |
| PGSC0003DMG400015923 | Acyl:coA ligase acetate-coA | |
| | synthetase | |
| PGSC0003DMG400015922 | Acyl:coA ligase acetate-coA | |
| | synthetase | |
| PGSC0003DMG400044288 | Acyltransferase | |
| PGSC0003DMG400015918 | Acyltransferase | 0.03 |

TABLE 7
Accession numbers, putative protein and co-expression
r-values-tomato, chromosome 12

| | | r-value of |
|---|---|---|
| | | correlation with |
| | | tomato GAME4 |
| Gene name | Putative protein | expression |
| Solyc12g006530 | Cycloartenol synthase | 0.08 |
| Solyc12g006520 | Cycloartenol synthase | 0.05 |
| Solyc12g006510 | Cycloartenol Synthase | −0.12 |
| Solyc12g006500 | Phosphate translocator protein | 0.15 |
| Solyc12g006490 | Beta-1-3-galactosyl-o- | 0.03 |
| | glycosyl-glycoprotein | |
| Solyc12g006480 | Nup205 protein | 0.35 |
| Solyc12g006470 | gamma-aminobutyrate | 0.94 |
| | Aminotransferase-like protein | |
| Solyc12g006460 | Cytochrome P450 (GAME 4) | 1.00 |
| Solyc12g006450 | gamma-aminobutyrate | −0.13 |
| | Aminotransferase-like protein | |
| Solyc12g006440 | Unknown Protein | 0.25 |
| Solyc12g006430 | UDP-glucuronosyltransferase | |
| | 1-1 82A1 | |
| Solyc12g006420 | Topoisomerase II-associated | 0.08 |
| | protein PAT1 | |
| Solyc12g006410 | UDP-arabinse 4-epimerase | |
| Solyc12g006400 | Unknown Protein | |
| Solyc12g006390 | 2-oxoglutarate-dependent | |
| | dioxygenase | |
| Solyc12g006380 | 2-oxoglutarate-dependent | 0.15 |
| | dioxygenase | |
| Solyc12g006370 | Amine oxidase family protein | −0.16 |
| Solyc12g006360 | Multidrug resistance protein | |
| | mdtK | |
| Solyc12g006350 | Auxin response factor 6 | 0.35 |
| Solyc12g006340 | Auxin response factor 6 | 0.47 |
| Solyc12g006330 | Acyltransferase-like protein | |
| Solyc12g006320 | ATP-dependent RNA helicase | 0.14 |
| Solyc12g006310 | Endoplasmic reticulum-Golgi | 0.25 |
| Solyc12g006300 | WD-repeat protein-like | −0.03 |
| Solyc12g006290 | Reticulon family protein | 0.19 |
| Solyc12g006280 | Myb-like DNA-binding protein | |

TABLE 8
Accession numbers, putative protein and co-expression r-values-potato, chromosome 12

| | | r-value of |
|---|---|---|
| | | correlation with |
| Gene name | Putative protein | potato GAME4 expression |
| PGSC0003DMG400020034 | Beta-amyrin synthase | −0.13 |
| PGSC0003DMG400024276 | Beta-Amyrin Synthase | −0.09 |
| PGSC0003DMG400024277 | Gene of unknown function | 0.10 |
| PGSC0003DMG400024278 | Phenylacetaldehyde synthase | 0.10 |
| PGSC0003DMG400024279 | Conserved gene of unknown | −0.16 |
| | function | |
| PGSC0003DMG400024280 | Triose phosphate/phosphate | −0.06 |
| | translocator, non-green plastid, | |
| | chloroplast | |
| PGSC0003DMG400024271 | Acetylglucosaminyltransferase | −0.06 |
| PGSC0003DMG400024273 | Resistance protein PSH-RGH6 | 0.37 |
| PGSC0003DMG400024281 | Gamma aminobutyrate | 0.94 |
| | transaminase isoform2 | |
| PGSC0003DMG400024274 | Cytochrome P450 | 1.00 |
| | monooxygenase GAME4 | |
| PGSC0003DMG400024275 | Gamma aminobutyrate | 0.37 |
| | transaminase isoform3 | |
| PGSC0003DMG400024282 | Fortune-1 | 0.36 |
| PGSC0003DMG400028806 | UDP-glycosyltransferase 82A1- | −0.18 |
| | like | |
| PGSC0003DMG401028807 | Topoisomerase II-associated | |
| | protein PAT1 | |
| PGSC0003DMG402028807 | UDP-arabinse 4-epimerase | |
| PGSC0003DMG400028824 | Gene of unknown function | |
| PGSC0003DMG400028808 | 2-oxoglutarate-dependent | −0.07 |
| | dioxygenase | |
| PGSC0003DMG400028809 | 2-oxoglutarate-dependent | 0.61 |
| | dioxygenase | |
| PGSC0003DMG400028810 | Amine oxidase | −0.04 |
| PGSC0003DMG400028825 | MATE transporter | |
| PGSC0003DMG400028826 | Auxin response factor 6 | |
| PGSC0003DMG400043090 | Integrase core domain containing | |
| | protein | |
| PGSC0003DMG400037700 | WRKY transcription factor 27 | |
| PGSC0003DMG400028811 | Acyltransferase | |
| PGSC0003DMG400028812 | DEAD-box ATP-dependent | 0.56 |
| | RNA helicase 53 | |
| PGSC0003DMG400028814 | WD-repeat protein | −0.10 |
| PGSC0003DMG401028829 | Polygalacturonase | |
| PGSC0003DMG400028815 | Reticulon family protein | 0.08 |
| PGSC0003DMG400028830 | Myb-like DNA-binding domain, | |
| | SHAQKYF class family protein | |

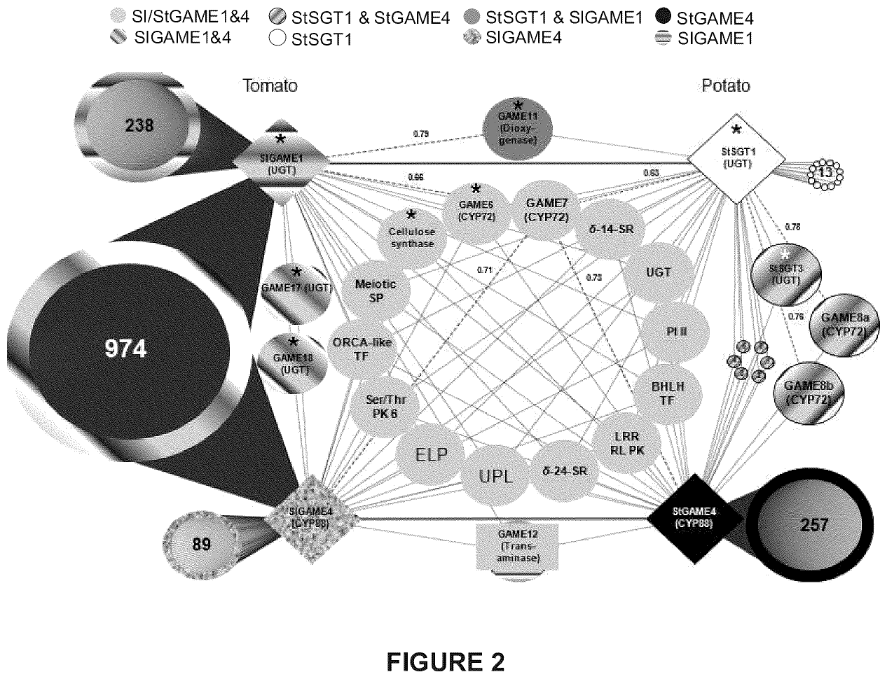

Sixteen genes from each species were co-expressed with GAME1/SGT1 (Table 9, FIG. 2). One of these genes, previously designated GLYCOALKALOID METABOLISM 4 (GAME4), encodes a member of the 88D subfamily of cytochrome P450 proteins (FIG. 3). GAME4 and GAME1/SGT1 display a very similar expression profile in tomato and potato (WO 2010/095843). The GAME1/SGT1 and GAME4 genes in tomato and potato are positioned in chromosomes 7 and 12 such that they are physically next to several of their co-expressed genes (FIG. 2).

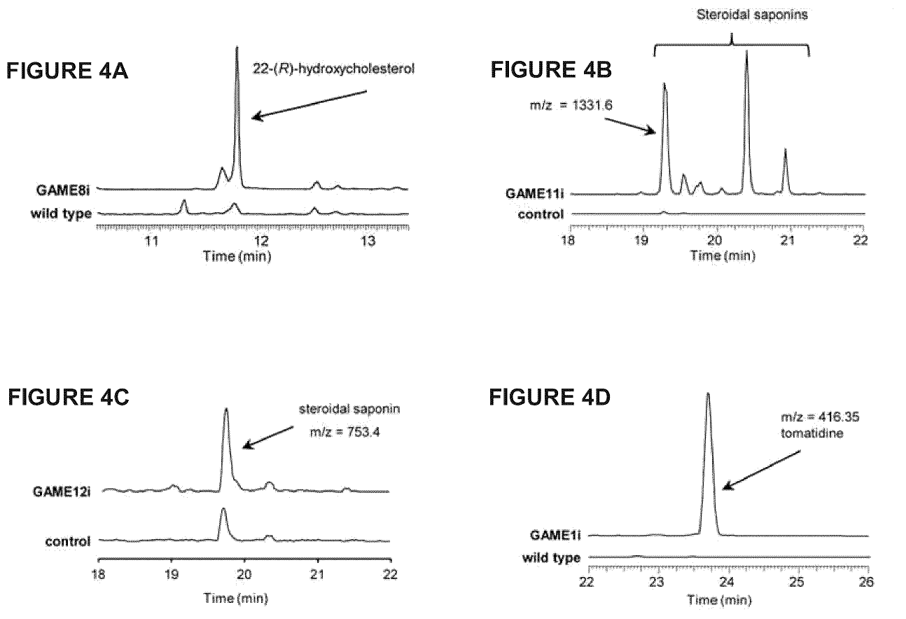

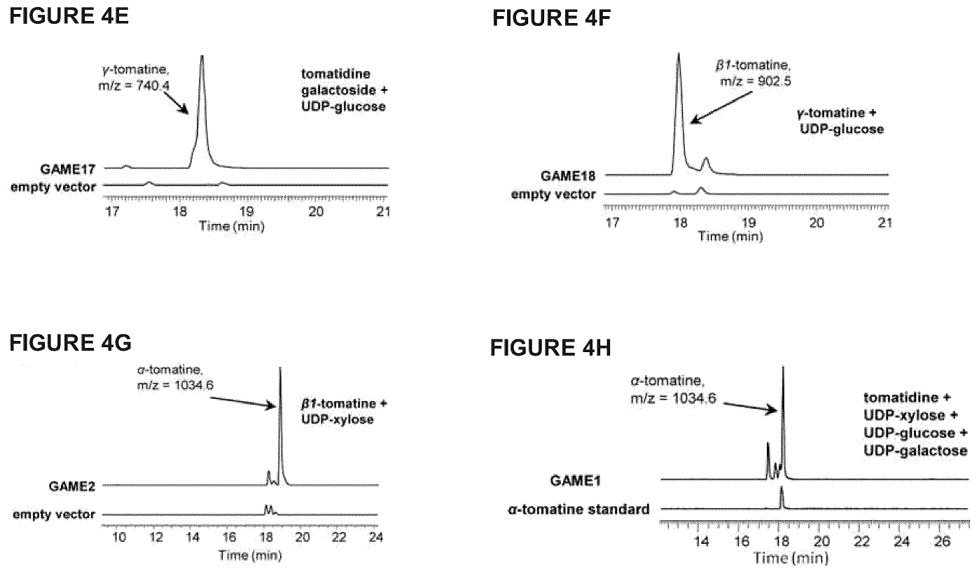

A cluster of GAME1/SGT1 co-expressed genes spans a ˜200Kbp genomic region on chromosome seven. Together with GAME1, the tomato cluster is composed of 7 co-expressed genes. These include 3 UDP-glycosyltransferases [GAME2 (termed SGT3 in potato); GAME17 and GAME18], a cytochrome P450 of the 72A subfamily (GAME6), a 2-oxoglutarate-dependent dioxygenase (GAME11), and a cellulose synthase-like protein (GAME15). It appears that in potato this cluster contains 5 co-expressed genes as it lacks homologs of the tomato genes encoding GAME17 and GAME18 UDP-glycosyltransferases. Enzyme activity assays were performed with the four recombinant clustered tomato UDP-glycosyltransferases. GAME17 and GAME18 exhibited UDP-glucosyltransferase activity when incubated with tomatidine galactoside (T-Gal) and y-tomatine (T-Gal-Glu) as a substrate, respectively, whereas GATE2 was shown to have an UDP-xylosyltransferase activity when incubated with β1-tomatine (T-Gal-Glu-Glu) as a substrate (FIGS. 4E, 4F, and 4G). GAME1 was previously shown to act as a tomatidine UDP-galactosyltransferase in tomato (Itkin et al., 2011, supra). When incubating the 4 recombinant UGT enzymes in a single test tube, with tomatidine, and all glycoside donors (UDP-galactose, -glucose and -xylose), the accumulation of the final SGA product α-tomatine was observed (FIG. 411).

Two genes encoding putative transcription factors were identified among the genes co-expressed with GAME1/SGT1 and GAME4 (FIG. 4): one gene, designated GAME9, was identified by the tomato TD Solyc01g090340 and by the potato ID PGS0003DMG400025989. It is described as ethylene-responsive element binding factor 13 and contains a putative AP2 domain. The other gene is the BHLH-transcription factor, identified by the tomato ID Solyc03g46570 and by the potato ID PGSC0003DMG400012262.

TABLE 9
Details of homologs co-expressed with known and putative steroidal alkaloid-
associated genes in both potato and tomato presented in FIG. 2
Tomato and potato sequences were obtained from Sol Genomics Network (solgenomics.net).
r-value for co-expression ≥0.8. TCON number, a contig reference name given by the inventors in the
assembly of RNAsec data. N/A, not available.

| Name | Tomato ID Solyc | Potato reads | Tomato ID |
|---|---|---|---|
| Extensin-like protein | Solyc01g006400 | PGSC0003DMG400023230 | TCONS_00007692 |
| GAME 9 | Solyc01g090340 | PGSC0003DMG400025989 | TCONS_00011729 |
| | [amino acid | [amino acid | |
| | SEQ ID NO: 13] | SEQ ID NO: 14] | |
| | [nucleic acid | [nucleic acid | |
| | SEQ ID NO: 15] | SEQ ID NO: 16] | |
| Delta (24)-sterol | Solyc02g069490 | PGSC0003DMG400021142 | TCONS_00044548 |
| reductase-like | | | |
| BHLH transcription | Solyc03g046570 | PGSC0003DMG400012262 | TCONS_00055879 |
| factor | [amino acid | [nucleic acid | |
| | SEQ ID NO: 17] | SEQ ID NO: 20] | |
| | [nucleic acid | [nucleic acid | |
| | SEQ ID NO: 18] | SEQ ID NO: 21] | |
| | [nucleic acid | | |
| | SEQ ID NO: 191 | | |
| LRR receptor-like | Solyc05g009100 | PGSC0003DMG400014576 | TCONS_00101281 |
| protein kinase | | | |
| Glycosyltransferase | Solyc05g053120 | PGSC0003DMG402027210 | TCONS 00100675 |
| Cellulose synthase- | Solyc07g043390 | PGSC0003DMG400011752 | TCONS 00135034 |
| like (GAME15) | | | |
| GAME6 (CYP72) | Solyc07g043460 | PGSC0003DMG400011750 | TCONS 00137734 |
| GAME1 | Solyc07g043490 | PGSC0003DMG400011749 | TCONS_00133014 |
| (Galactosyltransferase) | | | |
| GAME7 (CYP72) | Solyc07g062520 | PGSC0003DMG402012386 | TCONS 00132326 |
| | (GAME1 r-value 0.66; | (SGT1 r-value 0.63; | |
| | GAME4 r-value 0.71) | GAME4 r-value 0.73) | |
| Srt/Thr protein | Solyc08g066050 | PGSC0003DMG400025461 | TCONS 00151251 |
| kinase 6 | | | |
| Meiotic serine | Solyc08g077860 | PGSC0003DMG401012339 | TCONS 00149157 |
| proteinase | | | |
| Sterol reductase | Solyc09g009040 | PGSC0003DMG400002720 | TCONS 00162820 |
| Ubiquitin protein | Solyc 10g008410 | PGSC0003DMG400021683 | TCONS 00183263 |
| ligase | | | |
| Proteinase inhibitor II | Solyc 11g020960 | PGSC0003DMG402003479 | TCONS_00194999 |
| GAME4 (CYP88) | Solyc 12g006460 | PGSC0003DMG400024274 | TCONS 00210154 |
| Gamma-aminobutyrate | Solyc 12g006470 | PGSC0003DMG400024281 | |
| Aminotransferase-like | | | |
| protein (transaminase) | | | |
| (GAME12) | | | |
| Beta-solanine | #N/A | PGSC0003DMG400011740 | |
| rhamnosyltransferase | | | |
| (SGT3) | | | |
| 2-oxoglutarate- | Solyc07g043420 | PGSC0003DMG400011751 | |
| dependent | [amino acid | [amino acid | |
| dioxygenase | SEQ ID NO: 22] | SEQ ID NO: 23] | |
| (GAME11) | [nucleic acid | [nucleic acid | |
| | SEQ ID NO: 24] | SEQ ID NO: 26] | |
| | [nucleic acid | [nucleic acid | |
| | SEQ ID NO: 25] | SEQ ID NO: 27] | |
| GAME18 | Solyc07g043500 | #N/A | |
| (Glycosyltransferase) | | | |
| GAME17 | Solyc07g043480 | #N/A | |
| (Glycosyltransferase) | | | |

### Example 3: Functional Analysis of GAME9-Transcription Factor

GAME9-silencing (RNAi) and overexpressing (OX) constructs were created by introducing the corresponding GAME9 DNA fragments to pK7GWIWG2(JJ) and pJCV52 binary vectors, respectively. Transgenic tomato and potato lines transformed with the respective GAME9 silencing and overexpressing constructs were generated as previously described (Itkin et al., 2011, supra). Tissue extracts were prepared and analyzed as described in Itkin et al. (2011, supra). SEQ ID NO: 18 presents the sequence of the GAME9 RNAi silencing molecule. The metabolic profiling of steroidal alkaloids using UPLC-TQ-MS was performed on extracts obtained from leaves and/or tubers of transgenic and wild type tomato and/or potato plants. In extract obtained from potato tuber peels of potato lines in which the gene encoding GAME9 was silenced (GAME9-RNAi lines) a reduction in a-solanine and α-chaconine was observed (FIGS. 5A and 5B, respectively). Leaves from potato GAME9-overexpression lines contained higher levels of α-solanine (FIG. 5C) and α-chaconine (FIG. 5D) compared to the wild type. A similar accumulation pattern was observed in potato leaves, having reduced amounts of α-chaconine and α-solanine in RNAi lines and increased amounts of these steroidal alkaloids in lines overexpressing the GAME9-transcription factor (FIG. 6).

In tomato, leaves extract of a line overexpressing the GAME9-transcription factor (designated 5879) contained higher levels of α-tomatine compared to its amount in leaf extract obtained from wild type plants. On the contrary, down regulation of the expression of GAME9-transcription factor (line 5871) resulted in significant reduction of α-tomatine content.

### Example 4: Functional Characterization of the GAME Genes

### GAME11 Silenced Plants

Virus induced gene silencing (VIGS) is a commonly used technique allowing systemic silencing of genes in various organs of the plant (Dinesh-Kumar S P et al., 2003. Methods Mol Biol 236:287-294). SEQ ID NO: 19 presents the sequence of the GAME11 RNAi silencing molecule.

Analysis of tomato leaves with VIGS-silenced GAME11, a putative dioxygenase in the cluster, revealed a significant reduction in α-tomatine levels and accumulation of several cholestanol-type steroidal saponins.

Silencing of GAME11 dioxygenase in tomato results in depletion of α-tomatine levels in leaves (m/z=1034.5) (FIG. 8A) while accumulating cholestanol-type steroidal saponins [i.e. STSs; m/z=1331.6, 1333.6, 1199.6, 1201.6 (major saponins)] (FIG. 8B). FIG. 8C shows MS/MS spectrum of m/z=1331.6 (at 19.28 min.). FIG. 8D shows the fragmentation patterns of the saponin eluted at 19.28 min. and accumulating in GAME11-silenced leaves. The corresponding mass signals are marked with an asterisk on the MS/MS chromatogram in FIG. 8C. The elemental composition and fragmentation patterns show that the compounds are cholestanol-type saponins, lacking one hydroxy-group and the E-ring (in comparison to furostanol-type saponins), which results in fragmentation, involving multiple losses of water molecules instead of tautomerisation and McLafferty rearrangement of the E-ring.

### GAME18 Silenced Plants

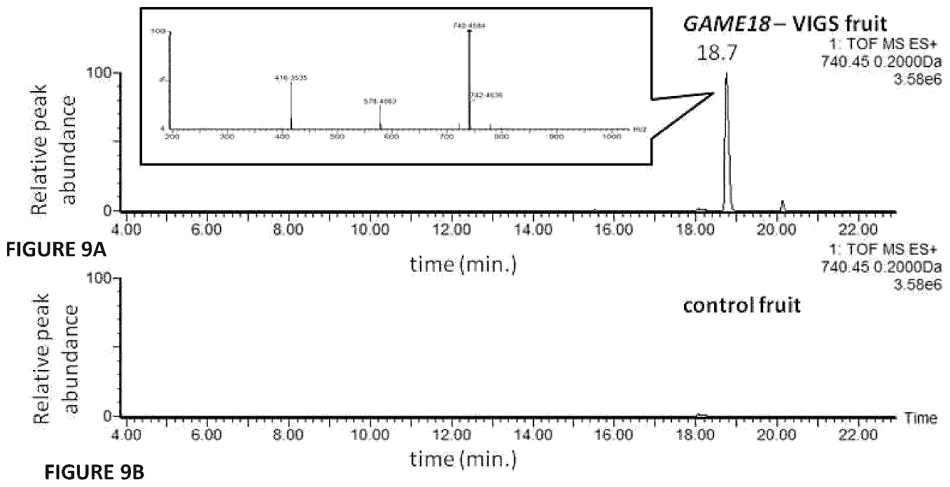

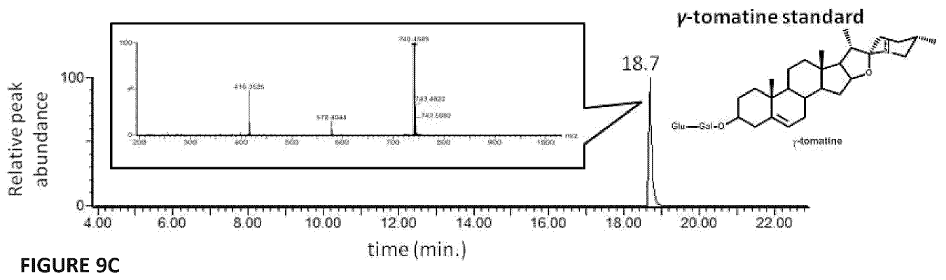

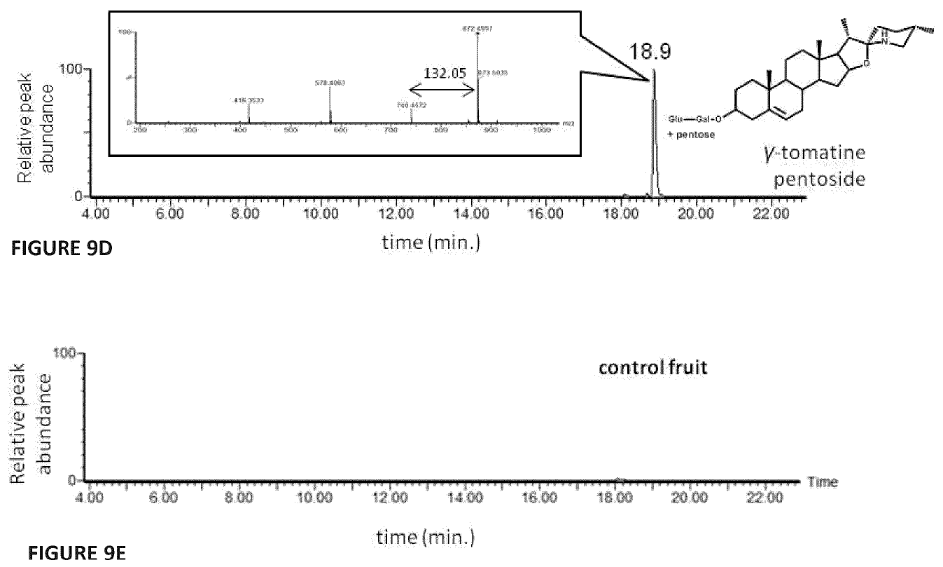

The role of GAME18 in creating the tetrasaccharide moiety of α-tomatine was supported by Virus Induced Gene Silencing (VIGS) assays as GAME18-silenced fruit accumulated y-tomatine which was not present in the control sample (FIG. 9).

Among the metabolites extracted from GAME18-silenced mature green fruit, peaks of newly accumulating compounds were detected, corresponding to the 7-tomatine standard (m/z=740.5) (FIGS. 9A-C), and 7-tomatine pentoside (m/z=872.5) (FIGS. 9D-9E).

### GAME12 Silenced Plants

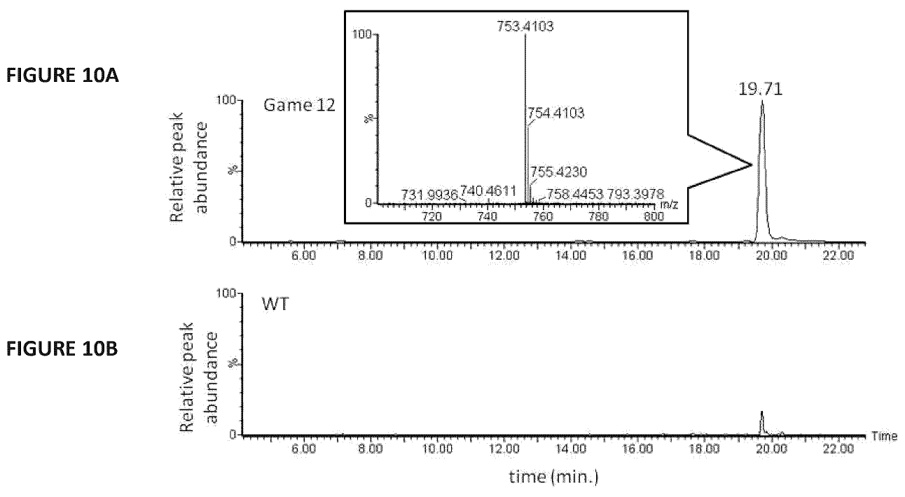

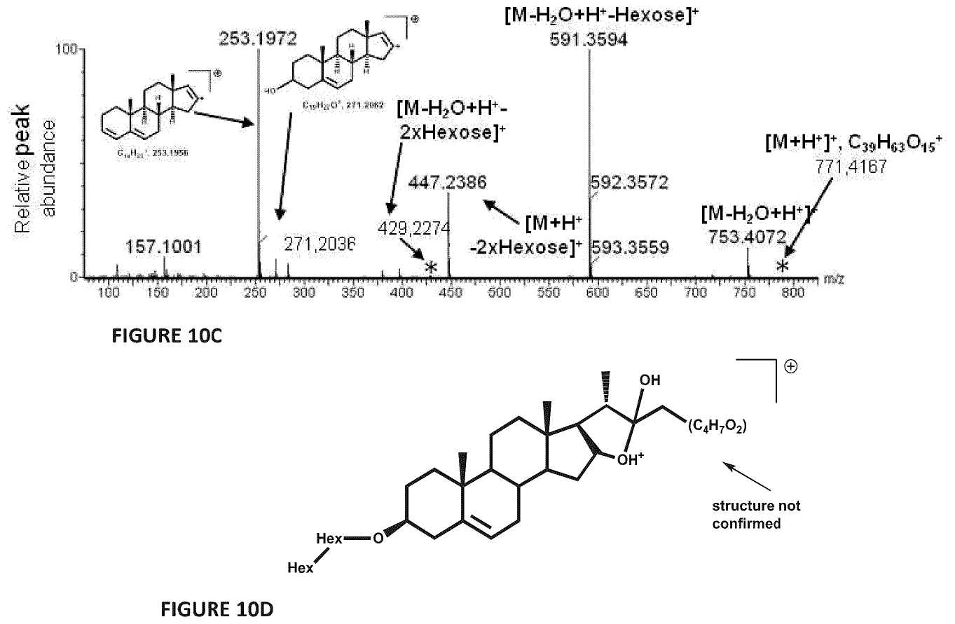

Silencing of GAME12 transaminase in tomato resulted in accumulation of a furastanol-type steroidal saponin (FIG. 4D). FIG. 10A shows that GAME12-silenced leaves accumulate an STS (m/z=753.4), while it exists in only minor quantities in wild type leaf FIG. 10B. FIG. 10C shows MS/MS spectrum of m/z=753.4 at 19.71 min. with interpretation of the fragments. Suggested structure of the STS at 19.71 min. is depicted in FIG. 10D, concluded from the characteristic mass fragments observed in the MS/MS experiment.

### Function of GAME7 and GAME8

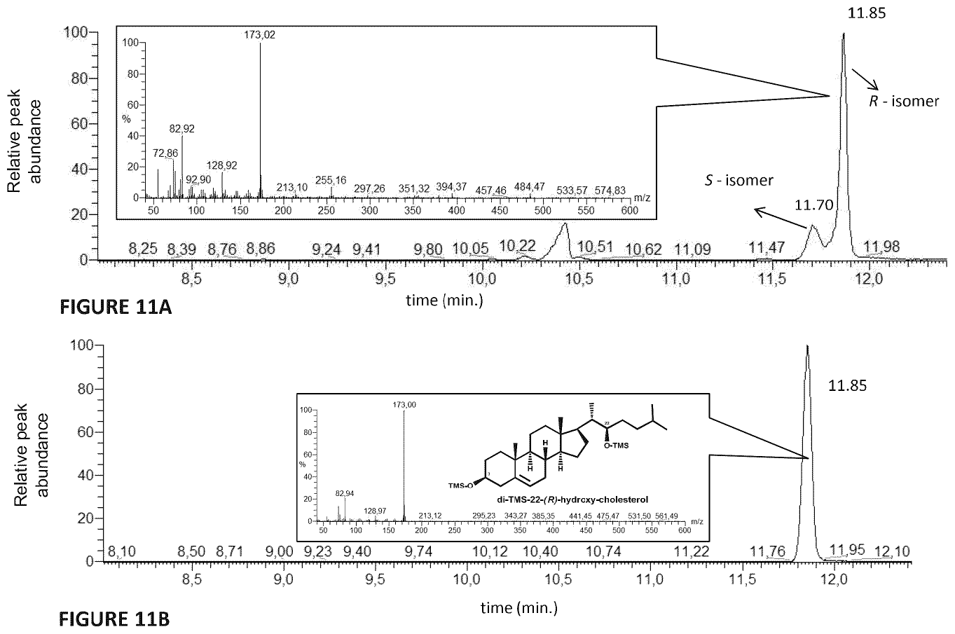

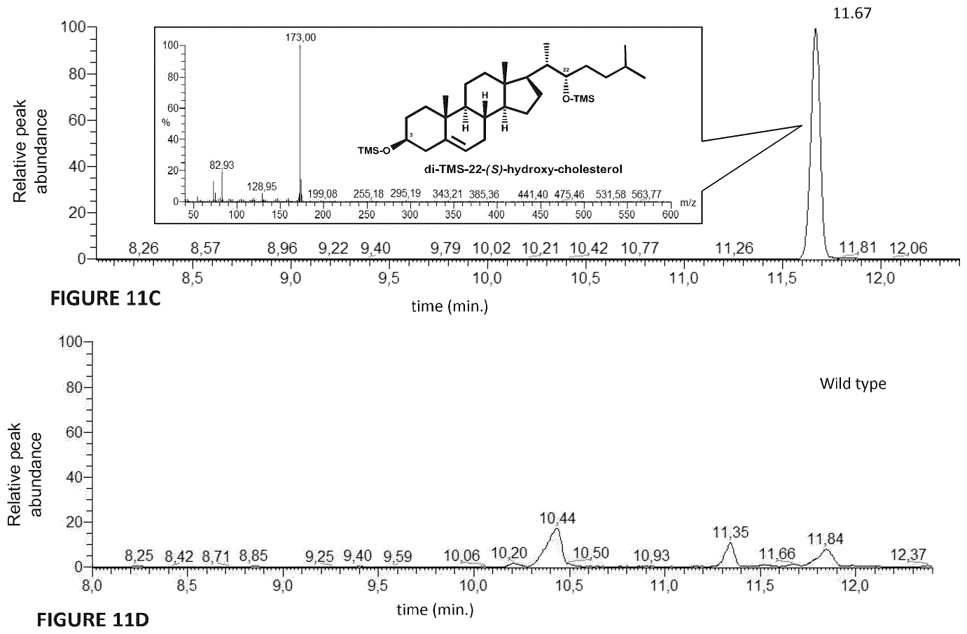

Genes that were tightly co-expressed and positioned elsewhere in the genome were also functionally examined. Two genes, designated GAME7 and GAME8 belong to the CYP72 subfamily of cytochrome P450s. GAME7 was co-expressed in both species (potato and tomato) while StGAME8a and StGAME8b were strongly co-expressed with StSGT1 and StGAME4 in potato. At present, we could not demonstrate SGA-related activity for GAME7 although as for GAME6 it was suggested to be involved in SGA metabolism (US 20120159676). Yet, GAME8-silenced tomato leaves accumulated 22-(R)-hydroxycholesterol (FIGS. 11A-11D), a proposed intermediate in the SGA biosynthetic pathway (FIG. 1). GAME8-silenced line accumulates both isomers in comparison to wild type (FIG. 11D). The (R)-isomer is more abundant and hence most likely to be the substrate of GAME8.

FIG. 12 shows the phylogenetic tree of GAME genes in the plant CYP450 protein family. The numbers on the branches indicate the fraction of bootstrap iterations supporting each node.

### Example 5: Proposed Biosynthetic Pathway in Solanaceous Plants

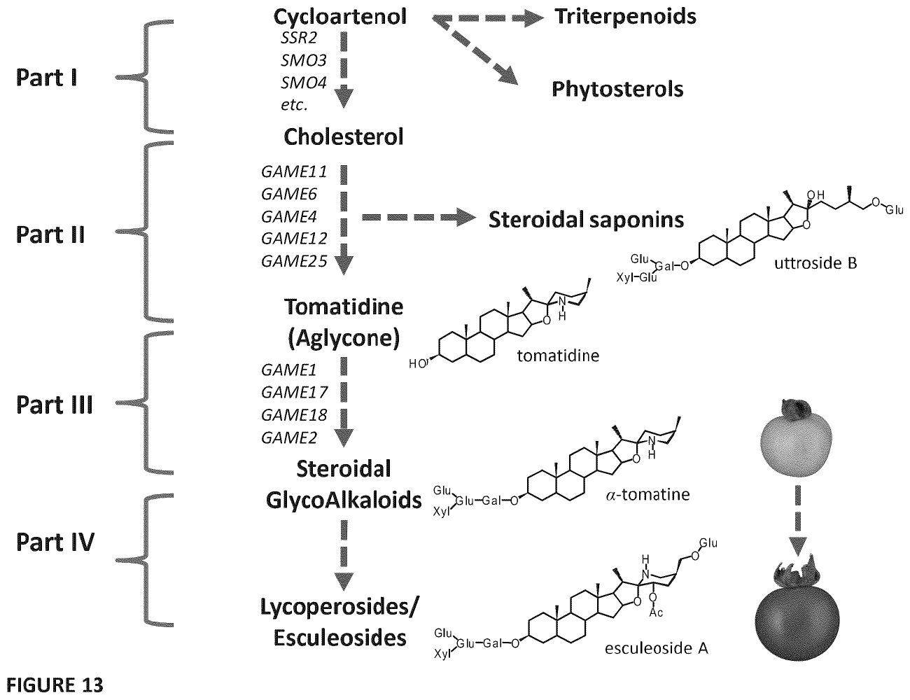

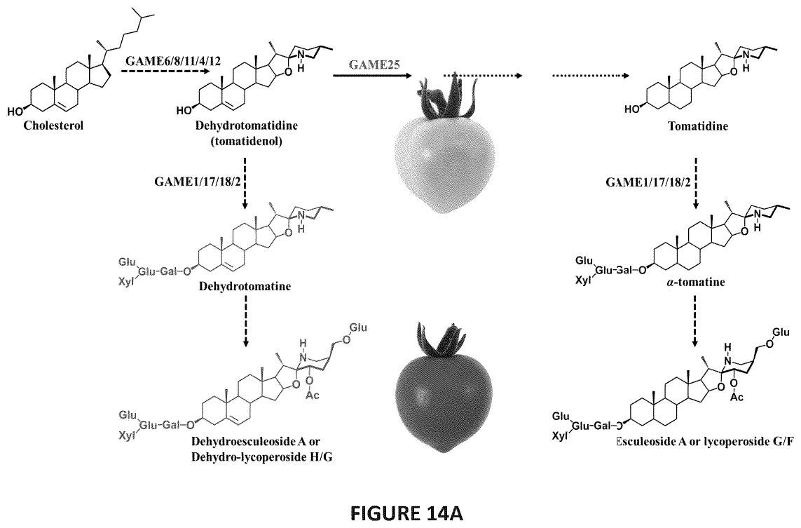

An expanded biosynthetic pathway in Solanaceous plants has been proposed, as depicted in the schematic of FIG. 13 (dashed arrows represent multiple enzymatic reactions in the pathway) with respect to the tomato. This pathway can be broken down into four parts for convenience. In Part I, a series of reactions (catalyzed, e.g., by SSR2, SMO3, SM04) converts cylcloartenol to cholesterol. Byproducts include triterpenoids and phytosterols. In Part II, a series of reactions (catalyzed, e.g., by GAME11, GAME6, GAME4, GAME12, GAME25) converts cholesterol to tomatidine (aglycone). Byproducts include steroidal saponins (e.g., uttroside B). In Part III, a series of reactions (catalyzed, e.g., by GAME1, GAME 17, GAME18, GAME2) converts tomatidine to steroidal glycoalkaloids (e.g., α-tomatine). In Part IV, a series of reactions converts steroidal glycoalkaloids (e.g., α-tomatine) of a green tomato to lycoperosides and/or esculeosides (e.g., esculeoside A) of a red tomato.

### Example 6. Pathways Involving Steroidal Glycoalkaloid (SGA) Biosynthesis in Tomato, Potato, and Eggplant

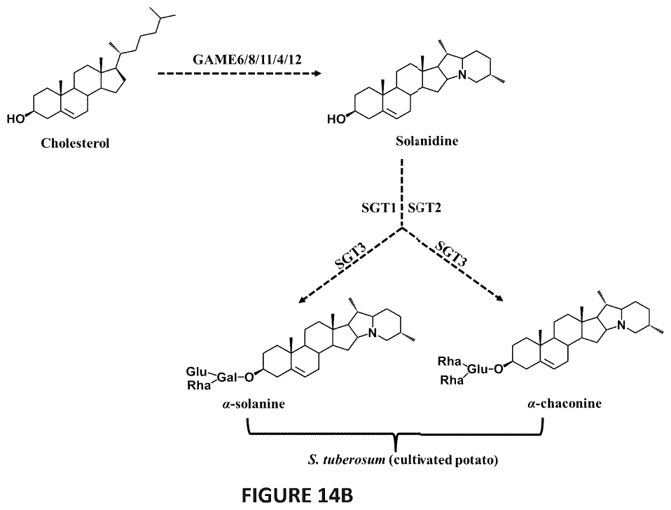

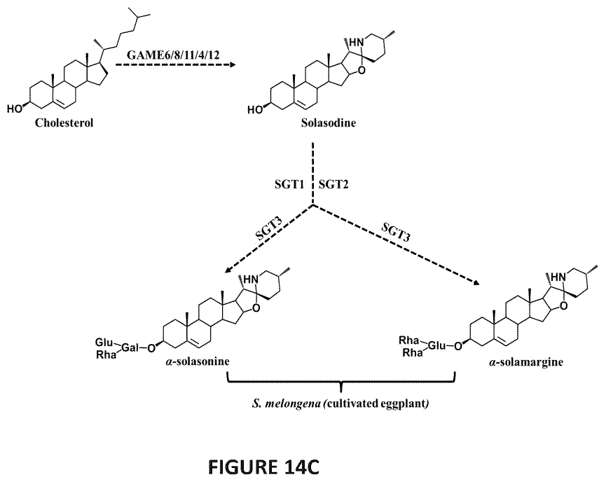

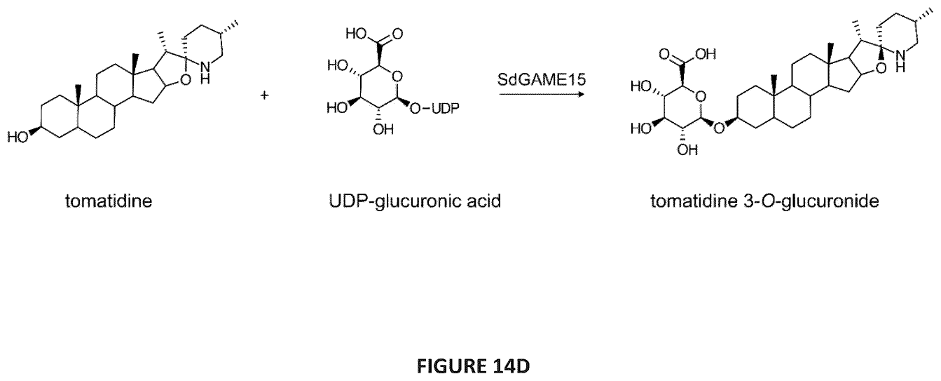

A cellulose synthase-like gene (GAME15) in tomato, potato, and eggplant has been identified as being associated with steroidal glycoalkaloid (SGA) biosynthesis (FIGS. 14A-14C). This gene has been shown to have been strongly co-expressed with other SGA biosynthesis genes (e.g., GAME4, GAME12) and also with regulators of SGA biosynthesis (e.g., GAME9). FIG. 14D provides a non-limiting example of a proposed pathway in Solanaceous plants, for example but not limited to, wherein a skilled artisan would recognize that the pathway encompasses production of tomatidine 3-O-glucoronide from tomatidine by a GAME15 glucuronic acid transferase activity. In certain embodiments, the GAME15 enzyme comprises a GAME15 present in a Solanaceous plant, for example but not limited to Solanum tuberosum, potato; Solanum lycopersicum, tomato; Solanum dulcamara, bitter-sweet; and Solanum melogena, eggplant.

Sequences were identified as follows:

### Cellulose Synthase Like_Tomato

[SEQ ID NO: 30]
ATGAAAAAAACCATGGAGCTCAACAAAAGCACTGTTCCACAACCTATCACCAC
CGTATACCGACTCCACATGTTCATCCACTCAATAATCATGCTTGCATTAATATACTACC
GTGTATCTAATTTGTTTAAATTCGAAAACATTCTCAGTTTACAAGCACTTGCTTGGGCG
CTCATCACTTTTGGTGAATTTAGTTTCATTCTCAAGTGGTTCTTCGGACAAGGTACTCG
TTGGCGCCCCGTTGAACGAGATGTTTTCCCTGAAAACATTACTTGCAAAGATTCCGAT
CTACCGCCAATTGACGTAATGGTATTCACTGCCAATCCTAAGAAAGAGCCAATTGTAG
ATGTCATGAACACTGTGATATCCGCAATGGCTCTTGATTATCCCACCGATAAATTGGC
TGTGTATCTCGCTGATGATGGAGGATGTCCATTGTCGTTGTACGCCATGGAACAAGCG
TGTTTGTTTGCAAAGCTATGGTTACCTTTCTGTAGAAACTATGGAATTAAAACGAGAT
GCCCAAAAGCATTTTTTTCTCCGTTAGGAGATGATGACCGTGTTCTTAAGAATGATGA
TTTTGCTGCTGAAATGAAAGAAATTAAATTGAAATATGAAGAGTTCCAGCAGAAGGT
GGAACATGCTGGTGAATCTGGAAAAATCAATGGTAACGTAGTGCCTGATAGAGCTTC
GCTTATTAAGGTAATAAACGAGAGGGAGAACGAAAAGAGTGTGGATGATATGACGA
AAATGCCCTTGCTAGTTTATGTATCCCGTGAAAGAAGATTCAACCGTCTTCATCATTTC
AAGGGTGGATCTGCAAATGCTCTACTTCGAGTTTCTGGAATAATGAGTAATGCCCCCT
ATGTACTGGTGTTAGATTGTGATTTCTTCTGTCATGATCCAATATCAGCTAGGAAGGC
AATGTGTTTTCATCTTGATCCAAAGCTATCATCTGATTTAGCCTATGTTCAGTTCCCTC
AAGTCTTTTACAATGTCAGCAAGTCAGATATTTATGATGTCAAAATTAGACAGGCTTA
CAAGACAATATGGCATGGAATGGATGGTATCCAAGGCCCAGTGTTATCTGGGACTGG
TTATTTTCTCAAGAGGAAAGCGTTATACACAAGTCCAGGAGTAAAAGAGGCGTATCTT
AGTTCACCGGAAAAGCATTTTGGAAGGAGTAAAAGGTTTCTTGCTTCATTAGAGGAG
AAAAATGGTTATGTTAAGGCAGATAAAGTCATATCAGAAGATATCATAGAGGAAGCT
AAGATGTTAGCTACTTGTGCATATGAGGATGGCACACATTGGGGTCAAGAGATTGGTT
ATTCATACGATTGTCATTTGGAGAGCACTTTTACTGGTTATCTATTACACTGCAAAGGG
TGGACATCTACTTATTTGTATCCAGACAGGCCATCTTTCTTGGGTTGTGCCCCAGTTGA
TATGCAAGGTTTCTCATCACAGCTCATCAAATGGGTTGCTGCACTTACACAAGCTGGT
TTATCACATCTCAATCCCATCACTTATGGTTTGAGTAGTAGGATGAGGACTCTCCAAT
GCATGTGCTATGCCTATTTGATGTATTTCACTCTTTATTCTTGGGGAATGGTTATGTAT
GCTAGTGTTCCTTCTATTGGCCTTTTGTTTGACTTCCAAGTCTATCCTGAGGTACATGA
TCCGTGGTTTGCAGTGTATGTGATTGCTTTCATATCGACAATTTTGGAGAATATGTCGG
AGTCAATTCCAGAAGGGGGATCAGTTAAAACGTGGTGGATGGAATACAGGGCATTGA
TGATGATGGGAGTTAGCGCAATATGGTTAGGAGGATTGAAAGCTATATATGACAAGA
TAGTCGGAACACAAGGAGAGAAATTGTATTTGTCGGACAAGGCAATTGACAAGGAAA
AGCTCAAGAAATACGAGAAGGGCAAATTTGATTTCCAAGGAATAGGGATACTTGCTC
TGCCACTGATAGCATTTTCCGTGTTGAACCTCGTAGGCTTCATTGTTGGAGCTAATCAT
GTCTTTATTACTATGAACTACGCAGGCGTGCTGGGCCAACTCCTCGTATCATCGTTCTT
CGTCTTTGTTGTCGTCACTGTTGTCATTGATGTTGTATCTTTCTTAAAGGTTTCTTAA

### Cellulose Synthase Like (Tomato)

[SEQ ID NO: 31]
MKKTMELNKSTVPQPITTVYRLHMFIHSIIMLALIYYRVSNLFKFENILSLQALAWA
LITFGEFSFILKWFFGQGTRWRPVERDVFPENITCKDSDLPPIDVMVFTANPKKEPIVDVMN
TVISAMALDYPTDKLAVYLADDGGCPLSLYAMEQACLFAKLWLPFCRNYGIKTRCPKAF
FSPLGDDDRVLKNDDFAAEMKEIKLKYEEFQQKVEHAGESGKINGNVVPDRASLIKVINE
RENEKSVDDMTKMPLLVYVSRERRFNRLHHFKGGSANALLRVSGIMSNAPYVLVLDCDF
FCHDPISARKAMCFHLDPKLSSDLAYVQFPQVFYNVSKSDIYDVKIRQAYKTIWHGMDGI
QGPVLSGTGYFLKRKALYTSPGVKEAYLSSPEKHFGRSKRFLASLEEKNGYVKADKVISE
DIIEEAKMLATCAYEDGTHWGQEIGYSYDCHLESTFTGYLLHCKGWTSTYLYPDRPSFLG
CAPVDMQGFSSQLIKWVAALTQAGLSHLNPITYGLSSRMRTLQCMCYAYLMYFTLYSWG
MVMYASVPSIGLLFDFQVYPEVHDPWFAVYVIAFISTILENMSESIPEGGSVKTWWMEYR
ALMMMGVSAIWLGGLKAIYDKIVGTQGEKLYLSDKAIDKEKLKKYEKGKFDFQGIGILAL
PLIAFSVLNLVGFIVGANHVFITMNYAGVLGQLLVSSFFVFVVVTVVIDVVSFLKVS

Cellulose Synthase Like (Solanum pennelii)

[SEQ ID NO: 32]
ATGAAAAAAACCATGGAGCTCAACAAAAGCACTGTTCCACAACCTATCACCAC
CGTATACCGACTCCACATGTTCATCCACTCAATAATCATGCTTGCATTAATATACTACC
GTGTATCTAATTTGTTTAAATTCGAAAACATTCTCAGTTTACAAGCACTTGCTTGGCTA
CTCATCACTTTTGGTGAATTTAGTTTCATTCTCAAGTGGTTCTTCGGACAAGGAACTCG
TTGGCGCCCCGTTGAACGAGATGTTTTCCCTGAAAACATTACTTGCAAAGATTCCGAT
CTACCGCCAATTGACGTAATGGTGTTCACTGCCAATCCTAAGAAAGAGCCAATTGTAG
ATGTCATGAACACTGTGATATCCGCAATGGCTCTTGATTATCCCACCGATAAATTGGC
TGTGTATCTGGCCGATGATGGAGGATGTCCATTGTCCTTGTACGCCATGGAACAAGCA
TGTTTGTTTGCAAAGCTATGGTTACCTTTCTGTAGAAAGTATGGAATTAAAACGAGAT
GCCCAAAAGCATTTTTTTCTCCGTTAGGAGATGATGACCGTGTTCTTAAGAATGATGA
TTTTGCTGCTGAAATGAAAGAAATTAAATTGAAATATGAAGAGTTCCAGCAGAACGT
GGAACATGCTGGTGAATCTGGAAAAATCAATGGCAACGTAGTGCCTGACAGAGCTTC
GCTTATTAAGGTAATAAACGAGAGGGAGAACGAAAAGAGTGTCGATGATTTAACGAA
AATGCCCTTGCTAGTTTATGTATCCCGTGAAAGAAGATTCAACCGTCTTCATCATTTCA
AGGGTGGATCTGCAAATGCTCTACTTCGAGTTTCTGGAATAATGAGTAATGCCCCCTA
TGTACTGGTGTTAGATTGTGATTTCTTCTGTCATGATCCGATATCAGCTAGGAAAGCA
ATGTGTTTTCATCTTGATCCAAAGCTATCATCTGATTTAGCCTATGTTCAGTTCCCTCA
AGTCTTTTACAATGTCAGCAAGTCCGATATTTATGATGTCAAAATTAGACAGGCTTAC
AAGACAATATGGCATGGAATGGATGGTATCCAAGGCCCAGTGTTATCTGGAACTGGT
TATTTTCTCAAGAGGAAGGCGTTATACACAAGTCCAGGAGTAAAAGAGGCGTATCTT
AGTTCACCGGAAAAGCATTTTGGAAGGAGTAAAAAGTTCCTTGCTTCATTAGAGGAG
AAAAATGGTTATGTTAAGGCAGATAAAGTCATATCAGAAGATATCATAGAGGAAGCT
AAGATCTTAGCTACTTGTGCATATGAGGATGGCACACATTGGGGTCAAGAGATTGGTT
ATTCATACGATTGTCATTTGGAGAGCACTTTTACTGGTTATCTATTACACTGCAAAGGG
TGGACATCTACTTATTTGTATCCAGACAGGCCATCTTTCTTGGGTTGTGCCCCAGTTGA
TATGCAAGGTTTCTCATCACAGCTCATAAAATGGGTTGCTGCACTTACACAAGCTGGT
CTATCACATCTCAATCCCATCACTTATGGTTTGAGTAGTAGGATGAGAACTCTCCAAT
GCATGTGCTATGCCTATTTGATGTATTTCACTCTTTATTCTTGGGGAATGGTTATGTAT
GCTAGTGTTCCTTCTATTGGCCTTTTGTTTGGCTTCCAAGTCTACCCTGAGGTACATGA
TCCATGGTTTGCAGTGTATGTGATTGCTTTCATATCGACAATTTTGGAGAATATGTCGG
AGTCAATTCCAGAAGGGGGATCAGTTAAAACGTGGTGGATGGAATACAGGGCATTGA
TGATGATGGGAGTTAGCGCAATATGGTTAGGAGGATTGAAAGCTATATATGACAAGA
TAGTCGGAACACAAGGAGAGAAATTGTATTTGTCGGACAAGGCAATTGACAAGGAAA
AGCTCAAGAAATACGAGAAGGGCAAATTTGATTTCCAAGGAATAGGGATACTTGCTC
TGCCATTGATAGCATTTTCCGTGTTGAACCTCGTAGGCTTCATTGTTGGAGCTAATCAT
GTCTTTATTACTATGAACTACGCAGGCGTGCTGGGCCAACTCCTCGTATCATCATTCTT
CGTCTTTGTTGTCGTCACTGTTGTCATTGATGTTGTATCTTTCTTAAAGGTTTCTTAA

Cellulose Synthase Like (Solanum pennellii)

[SEQ ID NO: 33]
MKKTMELNKSTVPQPITTVYRLHMFIHSIIMLALIYYRVSNLFKFENILSLQALAWL
LITFGEFSFILKWFFGQGTRWRPVERDVFPENITCKDSDLPPIDVMVFTANPKKEPIVDVMN
TVISAMALDYPTDKLAVYLADDGGCPLSLYAMEQACLFAKLWLPFCRKYGIKTRCPKAF
FSPLGDDDRVLKNDDFAAEMKEIKLKYEEFQQNVEHAGESGKINGNVVPDRASLIKVINE
RENEKSVDDLTKMPLLVYVSRERRFNRLHHFKGGSANALLRVSGIMSNAPYVLVLDCDFF
CHDPISARKAMCFHLDPKLSSDLAYVQFPQVFYNVSKSDIYDVKIRQAYKTIWHGMDGIQ
GPVLSGTGYFLKRKALYTSPGVKEAYLSSPEKHFGRSKKFLASLEEKNGYVKADKVISEDI
IEEAKILATCAYEDGTHWGQEIGYSYDCHLESTFTGYLLHCKGWTSTYLYPDRPSFLGCAP
VDMQGFSSQLIKWVAALTQAGLSHLNPITYGLSSRMRTLQCMCYAYLMYFTLYSWGMV
MYASVPSIGLLFGFQVYPEVHDPWFAVYVIAFISTILENMSESIPEGGSVKTWWMEYRAL
MMMGVSAIWLGGLKAIYDKIVGTQGEKLYLSDKAIDKEKLKKYEKGKFDFQGIGILALPL
IAFSVLNLVGFIVGANHVFITMNYAGVLGQLLVSSFFVFVVVTVVIDVVSFLKVS

### Cellulose Synthase Like (Potato)

[SEQ ID NO: 34]
ATGAAAAAAACCATGGAGCTCAACAAAAGCACTGTTCCACAACCTATCACCAC
CATATACCGACTCCACATGTTTATCCACTCTATAATCATGGTTGCATTAATATACTACC
GTGTATCTAATTTGTTTAAATTCGAAAACATTCTGAGTTTACAAGCACTTGCTTGGGTA
CTCATCACTTTTGGTGAATTTAGTTTCATTCTCAAGTGGTTCTTCGGACAAGGAACTCG
TTATCGCCCTGTTGAAAGAGATGTTTTCCCTGAAAACATAACTTGCAAAGATTCCGAT
CTACCACCAATTGACGTAATGGTATTCACTGCCAATCCTAAGAAAGAGCCAATTGTGG
ATGTCATGAACACTGTGATATCCGCAATGGCTCTTGATTATCCTACGGATAAATTGGC
TGTGTATCTGGCTGATGATGGAGGATGTCCTTTGTCATTGTACGCCATGGAAGAAGCA
TGTGTGTTTGCAAAGCTGTGGCTACCTTTCTGTAGGAAGTATGGAATTAAAACTAGAT
GCCCTAAAGCGTTTTTTTCTCCTTTAGGAGATGATGAACGTGTTCTTAAGAATGATGAT
TTTGATGCTGAAATGAAAGAAATTAAATTGAAATATGAAGAGTTCCAGCAGAATGTG
GAACGTGCTGGTGAATCTGGAAAAATCAATGGTAACGTAGTGCCTGATAGAGCCTCG
TTTATTAAGGTAATAAACGACAGAAAAGCGGAGAGCGAAAAGAGTGCCGATGATTTA
ACGAAAATGCCCTTGCTAGTTTATGTATCCCGTGAAAGAAGATTCAACCGTCTTCATC
ACTTCAAGGGTGGATCTGCAAATGCTCTTCTTCGAGTTTCTGGAATAATGAGTAATGC
CCCCTATATACTGGTGTTAGATTGTGATTTCTTCTGTCATGATCCAATATCAGCTAGGA
AGGCAATGTGTTTTCATCTTGATCCAAAGCTATCATCTGATTTAGCTTATGTTCAGTTC
CCTCAAGTCTTTTACAATGTCAGCAAGTCCGATATTTATGATGTCAAAATTAGACAGG
CTTACAAGACAATATGGCATGGAATGGATGGTATCCAAGGCCCAGTGTTATCAGGAA
CTGGTTATTTTCTGAAGAGGAAGGCGTTATACACGAGTCCAGGAGTAAAGGAGGAGT
ATCTTAGTTCACCGGAAAAGCATTTTGGAAGGAGTAAAAAGTTCCTTGCTTCACTAGA
GGAGAAAAATGGTTATGTTAAGGCAGAGAAAGTCATATCAGAAGATATCGTAGAGGA
AGCTAAGACCTTAGCTACTTGTGCATATGAGGATGGCACACATTGGGGTCAAGAGATT
GGTTATTCATACGATTGTCATTTGGAGAGCACTTTTACTGGTTATCTATTACACTGCAA
AGGGTGGAGATCGACTTATTTGTATCCAGACAGGCCATCTTTCTTGGGTTGTGCCCCA
GTTGATATGCAAGGTTTCTCCTCACAGCTCATAAAATGGGTTGCTGCACTTACACAAG
CTGGTTTATCACATCTCAATCCCATCACTTATGGCTTTAGTAGCAGGATGAAAACTCTC
CAATGCATGTGCTATGCCTATTTGATATATTTCACTCTTTATTCTTGGGGAATGGTTCT
ATATGCTAGTGTTCCTTCTATTGGCCTTTTGTTTGGCTTCCAAGTCTATCCCGATGTAC
ATGATCCATGGTTTGCAGTGTATGTGATTGCTTTCATATCGGCAATTTTGGAGAATATG
TCGGAGTCAATTCCTGATGGGGGATCATTTAAATCTTGGTGGATGGAATACAGGGCAC
TGATGATGATGGGAGTTAGTGCAATATGGTTAGGAGGATTGAAAGCTATATTAGACA
GGATAATCGGAACAGAAGGAGAGAAATTGTATTTATCGGACAAGGCAATTGACAAGG
AAAAGCTCAAGAAATACGAGAAGGGGAAATTTGATTTCCAAGGAATAGGGATACTTG
CTGTACCATTGATAGCATTTTCCTTGTTGAACCTCGTAGGCTTCATTGTTGGAGCTAAT
CATGTCTTTATTACTATGAACTACGCAGGTGTGCTTGGCCAACTCCTCGTATCATCCTT
CTTCGTCTTTGTCGTGGTCACTGTTGTCATTGATGTCGTTTCTTTCTTAAAGGTTTCTTA
A

### Cellulose Synthase Like (Potato)

[SEQ ID NO: 35]
MELNKSTVPQPITTIYRLHMFIHSIIMVALIYYRVSNLFKFENILSLQALAWVLITFG
EFSFILKWFFGQGTRYRPVERDVFPENITCKDSDLPPIDVMVFTANPKKEPIVDVMNTVISA
MALDYPTDKLAVYLADDGGCPLSLYAMEEACVFAKLWLPFCRKYGIKTRCPKAFFSPLG
DDERVLKNDDFDAEMKEIKLKYEEFQQNVERAGESGKINGNVVPDRASFIKVINDRKAES
EKSADDLTKMPLLVYVSRERRFNRLHHFKGGSANALLRVSGIMSNAPYILVLDCDFFCHD
PISARKAMCFHLDPKLSSDLAYVQFPQVFYNVSKSDIYDVKIRQAYKTIWHGMDGIQGPV
LSGTGYFLKRKALYTSPGVKEEYLSSPEKHFGRSKKFLASLEEKNGYVKAEKVISEDIVEE
AKTLATCAYEDGTHWGQEIGYSYDCHLESTFTGYLLHCKGWRSTYLYPDRPSFLGCAPV
DMQGFSSQLIKWVAALTQAGLSHLNPITYGFSSRMKTLQCMCYAYLIYFTLYSWGMVLY
ASVPSIGLLFGFQVYPDVHDPWFAVYVIAFISAILENMSESIPDGGSFKSWWMEYRALMM
MGVSAIWLGGLKAILDRIIGTEGEKLYLSDKAIDKEKLKKYEKGKFDFQGIGILAVPLIAFS
LLNLVGFIVGANHVFITMNYAGVLGQLLVSSFFVFVVVTVVIDVVSFLKVS

Cellulose Synthase Like (Solanum chacoense)

[SEQ ID NO: 36]
ATGAAAAAAACCATGGAGCTCAACAAAAGCACTGTTCCACAACCTATCACCAC
CATATACCGACTCCACATGTTCGTCCATTCTATAATCATGGCTGCATTAATATACTACC
GTGTATCTAATTTGTTTAAATTCGAAAACATTCTGAGTTTACAAGCACTTGCTTGGGTA
CTCATCACTTTTGGTGAATTTAGTTTCATTCTCAAGTGGTTCTTCGGACAAGGAACTCG
TTGGCGCCCTGTTGAAAGAGATGTTTTCCCTGAAAACATAACTTGCAAAGATTCCGAT
CTACCACCAATTGACGTAATGGTATTCACTGCCAATCCTAAGAAAGAGCCAATTGTGG
ATGTCATGAACACTGTGATATCCGCAATGGCTCTAGATTATCCTACGGATAAATTGGC
TGTGTATCTGGCTGATGATGGAGGATGTCCTTTGTCATTGTACGCCATGGAAGAAGCA
TGTGTGTTTGCAAAGCTGTGGCTACCTTTCTGTAGGAAGTATGGAATTAAAACCAGAT
GCCCTAAAGCGTTTTTTTCTCCTTTAGGAGATGATGACCGTGTTCTTAAGAATGATGAT
TTTGATGCTGAAATGAAAGAAATTAAATTGAAATATGAAGAGTTCCAGCAGAATGTG
GAACGTGCTGGTGAATCTGGAAAAATCAATGGTAACGTAGTGCCTGATAGAGCCTCG
TTTATTAAGGTAATAAACGACAGAAAAACGGAGAGCGAAAAGAGTGCCGATGATTTA
ACGAAAATGCCCTTGCTAGTTTATGTATCCCGTGAAAGAAGATTCAACCGTCTTCATC
ACTTCAAGGGTGGATCTGCAAATGCTCTTCTTCGAGTTTCTGGAATAATGAGTAATGC
CCCCTATATACTGGTGTTAGATTGTGATTTCTTCTGTCATGATCCAATATCAGCTAGGA
AGGCAATGTGTTTTCATCTTGATCCAAAGCTATCATCTGATTTAGCTTATGTTCAGTTC
CCTCAAGTCTTTTACAATGTCAGCAAGTCCGATATTTATGATGTCAAAATTAGACAGG
CTTACAAGACAATATGGCATGGAATGGATGGTATCCAAGGCCCAGTGTTATCAGGAA
CTGGTTATTTTCTGAAGAGGAAGGCGTTATACACGAGTCCAGGAGTAAAGGAGGAGT
ATCTTAGTTCACCGGAAAAGCATTTTGGAAGGAGTAAAAAGTTCCTTGCTTCACTAGA
GGAGAAAAATGGTTATGTTAAGGCAGAGAAAGTCATATCAGAAGATATCGTAGAGGA
AGCTAAGACCTTAGCTACTTGTGCATATGAGGATGGTACACATTGGGGTCAAGAGATC
GGTTATTCATACGATTGTCATTTGGAGAGCACTTTTACTGGTTATCTATTACACTGCAA
AGGGTGGACATCGACTTATTTGTATCCAGACAGGCCATCTTTCTTGGGTTGTGCTCCA
GTTGATATGCAAGGTTTCTCCTCACAGCTCATAAAATGGGTTGCTGCACTTACACAAG
CTGGTTTATCACATCTCAATCCCATCACTTATGGCTTGAGTAGCAGGATGAAAACTCT
CCAATGCATGTGCTATGCCTATTTGATATATTTCACTCTTTATTCTTGGGGAATGGTTC
TATATGCTAGTATTCCTTCTATTGGTCTTTTGTTTGGCTTCCAAGTCTATCCGGAGGTA
CATGATCCATGGTTTGCAGTGTATGTGATTGCTTTCATATCGACAATTTTGGAGAATAT
GTCGGAGTCAATTCCAGAAGGGGGATCATTTAAATCGTGGTGGATGGAATACAGGGC
ACTGATGATGATGGGAGTTAGTGCAATATGGTTAGGAGGATTGAAAGCTATATTAGA
CAAGATAATCGGAACAGAAGGAGAGAAATTGTATTTGTCAGACAAGGCAATTGACAA
GGAAAAGCTCAAGAAATACGAGAAGGGGAAATTTGATTTCCAAGGAATAGGGATACT
TGCTGTACCATTGATAGCATTTTCCCTGTTGAACCTGGTAGGCTTCATTGTTGGAGCTA
ATCATGTCTTTATTACTATGAACTACGCAGGTGTGCTTGGCCAACTCCTCGTATCATCC
TTCTTCGTCTTTGTCGTGGTCACTGTTGTCATTGATGTCGTTTCTTTCTTAAAGGTTTCT
TAA

Cellulose Synthase Like (Solanum chacoense)

[SEQ ID NO: 37]
MKKTMELNKSTVPQPITTIYRLHMFVHSIIMAALIYYRVSNLFKFENILSLQALAWV
LITFGEFSFILKWFFGQGTRWRPVERDVFPENITCKDSDLPPIDVMVFTANPKKEPIVDVMN
TVISAMALDYPTDKLAVYLADDGGCPLSLYAMEEACVFAKLWLPFCRKYGIKTRCPKAF
FSPLGDDDRVLKNDDFDAEMKEIKLKYEEFQQNVERAGESGKINGNVVPDRASFIKVIND
RKTESEKSADDLTKMPLLVYVSRERRFNRLHHFKGGSANALLRVSGIMSNAPYILVLDCD
FFCHDPISARKAMCFHLDPKLSSDLAYVQFPQVFYNVSKSDIYDVKIRQAYKTIWHGMDG
IQGPVLSGTGYFLKRKALYTSPGVKEEYLSSPEKHFGRSKKFLASLEEKNGYVKAEKVISE
DIVEEAKTLATCAYEDGTHWGQEIGYSYDCHLESTFTGYLLHCKGWTSTYLYPDRPSFLG
CAPVDMQGFSSQLIKWVAALTQAGLSHLNPITYGLSSRMKTLQCMCYAYLIYFTLYSWG
MVLYASIPSIGLLFGFQVYPEVHDPWFAVYVIAFISTILENMSESIPEGGSFKSWWMEYRAL
MMMGVSAIWLGGLKAILDKIIGTEGEKLYLSDKAIDKEKLKKYEKGKFDFQGIGILAVPLI
AFSLLNLVGFIVGANHVFITMNYAGVLGQLLVSSFFVFVVVTVVIDVVSFLKVS

### Cellulose Synthase Like (Eggplant)

[SEQ ID NO: 38]
ATGAAAAAACAAATGGAGCTCAACAGAAGTGTTGTACCGCAACCTATCACCAC
CATTTACCGTCTCCACATGTTTATCCATGCCCTAATCATGCTAGCACTAATATACTACC
GTGTCTCTAATTTGGCCAAATTCGAAAACATCCTCAGTTTACAAGCACTTGCTTGGGCT
CTTATCACGTTAGGTGAACTTTGTTTCATAGTCAAGTGGTTCTTCGGACAAGGGACTC
GTTGGCGTCCTGTTGATAGGGATGTCTTCCCTGAAAACATCACTTGTCCAGATTCCGA
GCTACCCCCCATTGATGTCATGGTTTTCACTGCAAATCCTAAGAAAGAGCCAATTGTG
GATGTCATGAACACTGTCATATCCGCAATGGCTCTTGATTACCCGACCGACAAATTGG
CCGTTTATTTGTCTGATGATGGAGGATGCCCCTTGACGTTGTACGCAATGGAGGAAGC
TTGTTCCTTTGCCAAGTTGTGGCTACCTTTTTGTAGGAAGTATGGAATCAAAACAAGG
TGCCCTAAGGCGTTTTTTTCTCCATTAGGAGAAGATGACCGTGTATTGAAGAGTGATG
ACTTTGTTTCTGAAATGAAAGAAATGAAGTCAAAATATGAAGAGTTCCAGCAGAACG
TGGACCGTGCTGGTGAATCCGGAAAAATCAAAGGTGACGTAGTGCCTGATAGACCCG
CGTTTCTTAAGGTACTAAATGACAGGAAGACGGAGAACGAGAAGAGTGCAGACGATT
TAACTAAAATGCCTTTGCTAGTATACGTATCCCGTGAAAGAAGAACTCACCGTCGCCA
TCACTTCAAGGGTGGATCTGCAAATGCTCTTCTTCGAGTTTCTGGGATAATCAGTAAT
GCCCCCTATATACTGGTTTTAGATTGTGATTTCTTCTGTCATGATCCAATATCAGCTCG
GAAGGCAATGTGTTTCCATCTTGATCCAAAACTATCACCTGACTTAGCTTACGTGCAG
TTCCCTCAAGTGTTTTACAATGTTAGCAAGTCCGATATTTACGACGTCAAAATTAGAC
AGGCTTACAAGACAATATGGCACGGGATGGATGGTATCCAAGGCCCAGTGTTATCGG
GAACTGGTTATTTTTTAAAAAAGAAGGCGTTGTACACGAGTCCAGGTCTAAAAGATG
AGTATCTTAGTTCACCGGAAAAGCATTTCGGAACGAGTAGAAAGTTCATTGCTTCACT
AGAGGAGAATAATTATGTTAAGCAAGAGAAAGTCATATCAGAAGATATCATAGAGGA
AGCTAAGAGACTGGCTACTTGTGCATACGAGGATGGCACACATTGGGGTCAAGAGGC
AAACAGGCCATCTTTCTTGGGTTGTGCCCCAGTTGATATGCAAGGTTTCTCCTCACAGC
TCATAAAATGGGTTGCTGCACTCACACAAGCAGGTCTATCACATCTCAATCCCATCAC
TTACGGCTTCAAGAGCAGAATGAGAACTCTCCAAGTCTTGTGTTATGCCTATTTGATG
TATTTCTCTCTTTATTCTTGGGGAATGGTTCTACATGCTAGTGTTCCTTCTATTGGCCTT
CTCTCTGGCATTAAAATCTACCCGGAGGTGTATGATCCATGGTTTGTTGTGTATGTGAT
TGCTTTCATATCAACAATTTTGGAGAATATGTCGGAATCAATTCCGGAAGGGGGATCG
GTTAAAACGTGGTGGATGGAATACAGGGCACTGATGATGATGGGAGTTAGTGCAATA
TGGCTAGGAGGAGTGAAAGCCATAGTAGACAAGATCATCGGAACGCAAGGAGAGAA
ATTGTATTTGTCGGACAAAGCAATTGACAAGGAAAAGCTCAAGAAATACGAGAAGGG
GAAATTTGATTTCCAAGGAATAGGAATACTTGCTGTACCATTGATAACATTTTCTGTGT
TGAACCTGGTAGGCTTCTTGGTTGGAATTAATCAAGTGTTGATAACGATGAAGTTCGC
AGGCGTGCTGGGCCAACTCCTCGTATCATCCTTCTTCGTCTTTGTCGTCGTTACTGTTG
TCATTGATGTCGTATCTTTCTTAAAGGATTCTTAA

### Cellulose Synthase Like (Eggplant)

[SEQ ID NO: 39]
MKKQMELNRSVVPQPITTIYRLHMFIHALIMLALIYYRVSNLAKFENILSLQALAW
ALITLGELCFIVKWFFGQGTRWRPVDRDVFPENITCPDSELPPIDVMVFTANPKKEPIVDV
MNTVISAMALDYPTDKLAVYLSDDGGCPLTLYAMEEACSFAKLWLPFCRKYGIKTRCPK
AFFSPLGEDDRVLKSDDFVSEMKEMKSKYEEFQQNVDRAGESGKIKGDVVPDRPAFLKV
LNDRKTENEKSADDLTKMPLLVYVSRERRTHRRHHFKGGSANALLRVSGIISNAPYIL VL
DCDFFCHDPISARKAMCFHLDPKLSPDLAYVQFPQVFYNVSKSDIYDVKIRQAYKTIWHG
MDGIQGPVLSGTGYFLKKKALYTSPGLKDEYLSSPEKHFGTSRKFIASLEENNYVKQEKVI
SEDIIEEAKRLATCAYEDGTHWGQEANRPSFLGCAPVDMQGFSSQLIKWVAALTQAGLSH
LNPITYGFKSRMRTLQVLCYAYLMYFSLYSWGMVLHASVPSIGLLSGIKIYPEVYDPWFV
VYVIAFISTILENMSESIPEGGSVKTWWMEYRALMMMGVSAIWLGGVKAIVDKIIGTQGE
KLYLSDKAIDKEKLKKYEKGKFDFQGIGILAVPLITFSVLNLVGFLVGINQVLITMKFAGVL
GQLLVSSFFVFVVVTVVIDVVSFLKDS

Cellulose Synthase Like (Capsicum annuum)

[SEQ ID NO: 40]
ATGGAGCTCAACAGATGTACGGTGCAGCAACCTACCACTGCCATATACCGACT
ACACATGTTTCTCCACTCTCTAATCATGCTTGCATTAGTATACTATCGTTTGTCTAATCT
GTTTTACTTCGAAAACGTCCTCACTTTACAAGCATTTGCATGGGGGCTTATCACCTTAG
GTGAAATTTGTTTCATTGTCAAGTGGTTCTTTGGTCAAGGGACTCGTTGGCGCCCCGTT
GTCAGGGAAGTGTTCCTGGACAATATTACTTGCCAAGATTCCGAGCTGCCCGCACTAG
ATGTGATGGTTTTCACTGCCAATCCCAAGAAAGAGCCAATTGTGGATGTCATGAACAC
TGTGATATCCGCAATGGCTCTTGATTACCCGACGGATAAATTGGCTGTGTATCTGGCT
GATGATGGAGGATGCCCCTTGACGTTGTACGCCATGGAGGAGGCCTGTTCTTTTGCCA
AGTTGTGGCTACCTTTCTGTAGGAAGTATGGAATCAAAACAAGGTGCCCCAAAGCGTT
TTTTTCTCCATTAGGAGAAGATGATCGTATCCTTAAGAACGATGACTTTGTAGCTGAA
ATGAAAGAAATTAAATTAAAATATGAGGAGTTCCAGCAGAATGTAAACCTTGCTGGT
GAATCCGGAAAAATCAAAGGTGACGTAGTGCCTGATAGAGCCTCGTTTATTAAGGTA
ATAAATGACAGGAAAATGGAGAACAAGAAGAGTGCCGACGATATAACGAAAATGCC
TTTGCTAGTATACGTATCCCGTGAAAGAAGATTTAACAGTCGTCATCACTTCAAGGGT
GGATCTGCAAATGCTCTTCTTCGAGTTTCAGGGATAATGAGTAATGCCCCCTATTTACT
GGTCTTAGATTGTGATTTCTTCTGTCATGATCCAACATCAGCTCGGAAGGCAATGTGTT
TCCATCTTGATCCAAAACTATCACCTTCCTTAGCTTATGTGCAGTTCCCTCAAGTGTTT
TACAATGTCAGCAAGTCCGATATATACGATGTCAAAATTAGACAGGCTTACAAGACA
ATATGGCACGGAATGGATGGTATCCAAGGCCCAGTGTTATCGGGAACTGGGTATTTTC
TGAAGAGGAAAGCGTTATACACGAGTCCAGGTCTAAAGGATGAGTATCTTATTTCACC
GGAAAAGCATTTCGGATCAAGTAGAAAGTTCATTGCTTCTCTAGAGGAGAACAATGG
TTATGTTAAGCAAGAGAAACTCATAACAGAAGATATTATAGAGGAAGCGAAGACCTT
GTCTACTTGTGCATACGAGGATGGTACACGATGGGGCGAAGAGATCGGTTATACCTA
CAATTGCCATTTGGAGAGCACTTTTACCGGCTATCTTTTGCACTGCAAAGGGTGGACA
TCAACATATTTGTATCCAGAAAGGCCATCTTTCTTGGGTTGTGCCCCAGTTGATATGCA
AGGATTCTCCTCACAACTCACAAAATGGGTTGCTGCACTCACACAAGCTGGTCTATCA
CATCTCAATCCCATCACTTACGGCATGAAGAGCAGGATTAAGACTATCCAATGCTTGT
GCTATGCCTATTTGATGTATTTCTCTCTCTATTCTTGGGGAATGGTTCTGCATGCTAGT
GTTCCTTCTATTAGCCTTTTGCTTGGCATTCAAGTCTACCCCGAGGTCTATGATCCATG
GTTTGCAGTGTATGTGCTTGCTTTCATATCGACAATTTTGGAGAACATGTCAGAGTCA
ATTCCAGAAGGCGGTTCAGTTAAAACTTGGTGGATGGAATACAGGGCACTGATGATG
ATGGGAGTTAGTGCAATATGGTTAGGAGGAGTGAAAGCTATAGTAGAAAAGATCATC
GGAACTCAAGGAGAGAAATTATATTTGTCGGACAAAGCAATTGACAAGGAAAAGCTC
AAGAAATATGAGAAGGGGAAATTTGATTTCCAAGGGATAGGGATACTTGCTGTTCCA
TTGATAACATTCTCAGCGTTGAATTTGGTAGGCTTCATGGTTGGAGCTAATCAAGTGA
TTCTTACTATGAAGTTCGAAGCTTTGCTAGGCCAACTCCTTGTGTCATCCTTCTTCGTC
TTTGTGGTGGTCACCGTTGTCATAGATGTCCTATCTTTCTTAAAAGACTCTTAA

Cellulose Synthase Like (Capsicum annuum)

[SEQ ID NO: 41]
MELNRCTVQQPTTAIYRLHMFLHSLIMLALVYYRLSNLFYFENVLTLQA
FAWGLITLGEICFIVKWFFGQGTRWRPVVREVFLDNITCQDSELPALDV
MVFTANPKKEPIVDVMNTVISAMALDYPTDKLAVYLADDGGCPLTLYAM
EEACSFAKLWLPFCRKYGIKTRCPKAFFSPLGEDDRILKNDDFVAEMKE
IKLKYEEFQQNVNLAGESGKIKGDVVPDRASFIKVINDRKMENKKSADD
ITKMPLLVYVSRERRFNSRHHFKGGSANALLRVSGIMSNAPYLLVLDCD
FFCHDPTSARKAMCFHLDPKLSPSLAYVQFPQVFYNVSKSDIYDVKIRQ
AYKTIWHGMDGIQGPVLSGTGYFLKRKALYTSPGLKDEYLISPEKHFGS
SRKFIASLEENNGYVKQEKLITEDIIEEAKTLSTCAYEDGTRWGEEIGY
TYNCHLESTFTGYLLHCKGWTSTYLYPERPSFLGCAPVDMQGFSSQLTK
WVAALTQAGLSHLNPITYGMKSRIKTIQCLCYAYLMYFSLYSWGMVLHA
SVPSISLLLGIQVYPEVYDPWFAVYVLAFISTILENMSESIPEGGSVKT
WWMEYRALMMMGVSAIWLGGVKAIVEKIIGTQGEKLYLSDKAIDKEKLK
KYEKGKFDFQGIGILAVPLITFSALNLVGFMVGANQVILTMKFEALLGQ
LLVSSFFVFVVVTVVIDVLSFLKDS

The following sequences were generated for silencing GAME15 in their respective plants:

### Region Used for GAME15 Silencing in Tomato

[SEQ ID NO: 42]
GGCTCTTGATTATCCCACCGATAAATTGGCTGTGTATCTCGCTGATGAT
GGAGGATGTCCATTGTCGTTGTACGCCATGGAACAAGCGTGTTTGTTTG
CAAAGCTATGGTTACCTTTCTGTAGAAACTATGGAATTAAAACGAGATG
CCCAAAAGCATTTTTTTCTCCGTTAGGAGATGATGACCGTGTTCTTAAG
AATGATGATTTTGCTGCTGAAATGAAAGAAATTAAATTGAAATATGAAG
AGTTCCAGCAGAAGGTGGAACATGC

### Region Used for GAME15 Silencing in Potato

[SEQ ID NO: 43]
GGCTCTTGATTATCCTACGGATAAATTGGCTGTGTATCTGGCTGATGAT
GGAGGATGTCCTTTGTCATTGTACGCCATGGAAGAAGCATGTGTGTTTG
CAAAGCTGTGGCTACCTTTCTGTAGGAAGTATGGAATTAAAACTAGATG
CCCTAAAGCGTTTTTTTCTCCTTTAGGAGATGATGAACGTGTTCTTAAG
AATGATGATTTTGATGCTGAAATGAAAGAAATTAAATTGAAATATGAAG
AGTTCCAGCAGAATGTGGAACGTGCTGGTG

### Region Used for GAME15 Silencing in Eggplant

[SEQ ID NO: 44]
GGCTCTTGATTACCCGACCGACAAATTGGCCGTTTATTTGTCTGATGAT
GGAGGATGCCCCTTGACGTTGTACGCAATGGAGGAAGCTTGTTCCTTTG
CCAAGTTGTGGCTACCTTTTTGTAGGAAGTATGGAATCAAAACAAGGTG
CCCTAAGGCGTTTTTTTCTCCATTAGGAGAAGATGACCGTGTATTGAAG
AGTGATGACTTTGTTTCTGAAATGAAAGAAATGAAGTCAAAATATGAAG
AGTTCCAGCAGAACGTGGACCGTGCTGGTGAATCCGGAAAAATCAAAGG
TGACGTAGTGCCTGATAGACCCGCGTTTCTTAAGGTACTAAATGACAGG
AAGACGGAGAACGAGAAGAGTGCAGACGATTTAACTAAAATGCCTTTGC
TAGTATACGTATCCCGTGAAAGAAGAACTCACCGTCGCCATCACTTCAA
GGGTGG

RNAi lines for the GAME15 gene in tomato and potato were generated. GAME15-RNAi transgenic tomato plants showed severe reduction in α-tomatine and downstream SGAs in leaves; α-tomatine was not detected in GAME15-silenced green fruit. Furthermore, no esculeosides or other SGAs were detected during tomato fruit developmental stages (e.g., breaker and red fruit). In addition, a 15-20 fold increase in cholesterol, which is a precursor for SGAs was observed in leaves and green fruit of GAME15-RNAi tomato plants. In potato, silencing of GAME1S resulted in a major reduction in α-chaconine and α-solanine, while the cholesterol pool in these lines increased.

### Example 7: Generation of GAME1S-RNAi Transgenic Tomato Potato and Eggplant Plants

The GAME15-RNAi construct was generated by introducing a selected fragment (silencing sequences SEQ ID NO: 42 (tomato), SEQ ID NO: 43 (potato), and SEQ ID NO: 44 (eggplant)) to pENTR/D-TOPO (Invitrogen) (by NotI and AscI) and further subcloning of this fragment to the pK7GWIWG2 (II) binary vector using the Gateway LR Clonase II enzyme mix (Invitrogen). The vector was transformed into tomato, potato and eggplant as described previously (Itkin et al. 2011. The Plant Cell 23:4507-25; Sonawane et al. 2018. PNAS 115(23): E5418-E5428). Positive GAME25-downregulated lines were further used for LC-MS analysis.

### Example 8: GAME15-Silenced Tomato Plants Showed Severely Reduced SGA Profile

In order to determine the precise role of GAME15 in SGA metabolism, GAME15-RNAi (GAME15i) transgenic tomato lines (#21, #22 and #23) were generated using the tomato silencing sequence above (SEQ ID NO: 42).

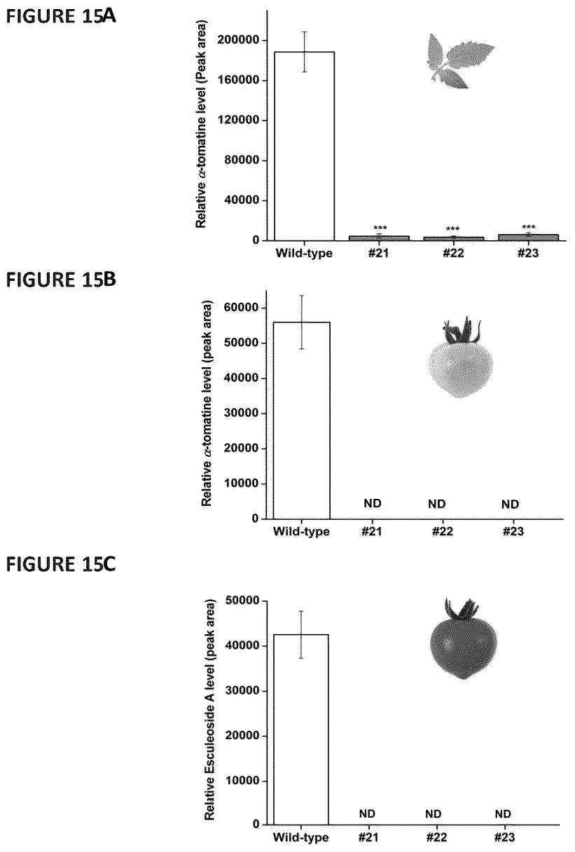

GAME15-RNAi leaves showed severe reduction in α-tomatine, compared with wild-type tomato leaves (FIG. 15A). Furthermore, the SGAs profile of GAME15i fruit was subsequently compared to wild-type ones at different stages of development and ripening. During the transition from green to red fruit in tomato, α-tomatine is converted to esculeosides and lycoperosides, while dehydrotomatine is converted to dehydroesculeosides and dehydrolycoperosides (FIG. 14A).

GAME15i green and red fruits did not show any trace of SGAs (e.g., α-tomatine or Esculeoside A) suggesting complete loss of SGAs in tomato fruits due to GAME15i silencing (FIGS. 15B and 15C).

### Example 9: Altering GAME15 Expression has Major Impact on SGAs in Potato

Similar to tomato, GAME15i was also silenced in potato (#1, #2, and #3) to determine its effect on potato SGAs metabolism, using the potato silencing sequence above (SEQ ID NO: 43).

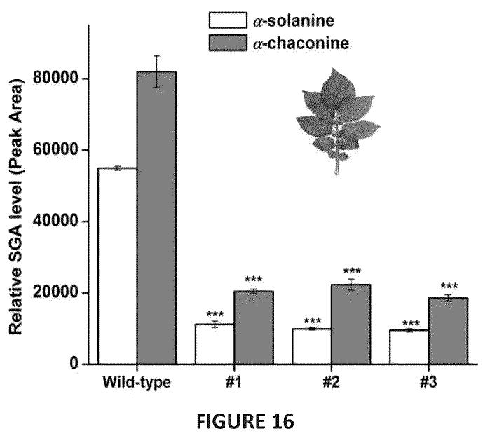

Silencing of GAME15 in potato resulted in drastic reduction in α-chaconine (shaded bars) and a-solanine (open bars), major SGAs in potato leaf tissue (FIG. 16), in comparison with potato leaf tissue of the wild-type.

### Example 10: High Cholesterol Accumulation in GAME15-Silenced Tomato Leaves

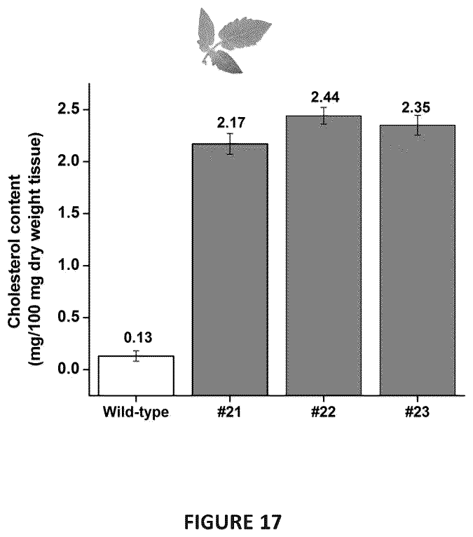

Cholesterol serves as a key precursor in the biosynthesis of SGAs (Sonawane et al., 2016, Nat. Plants 3: 16205). As severe reduction and subsequent complete loss of SGAs was observed in GAME15i-silenced tomato plants, the cholesterol levels in these plants were examined. An ˜15-20-fold increase in cholesterol (SGA precursor) was observed in leaves of GAME15i-silenced tomato plants compared to the leaves of wild-type tomato plants (FIG. 17).

### Example 11: Altering GAME15 Expression and Observing its Impact on SGAs in Eggplant

Similar to potato, GAME15i is also silenced in eggplant to determine its effect on potato SGAs metabolism, using the eggplant silencing sequence above (SEQ ID NO: 44).

The effect of silencing of GAME15 in eggplant is observed with respect to reduced levels of a-solasonine and/or α-solamargine in comparison with wild-type eggplant (FIG. 14C).

### Example 12: Overexpression of GAME15 in Tomato, Potato, and Eggplant

Alternatively, tomato, potato, and/or eggplant plants are genetically modified, or gene edited to overexpress GAME15.

To increase production of α-tomatine and esculeosides and/or lycoperosides in tomato plants (FIG. 14A), tomato plants are genetically modified, or gene edited to overexpress GAME15.

To increase production of α-solanine and/or α-chaconine in potato plants (FIG. 14B), potato plants are genetically modified, or gene edited to overexpress GAME15.

To increase production of α-solasonine and/or α-solamargine in eggplant (FIG. 14C), eggplant plants are genetically modified, or gene edited to overexpress GAME15.

### Example 13: Plants and Crops with Modified Levels and Compositions of SGAs

Based on the foregoing, Solanaceous plants (e.g., tomato, potato, eggplant, and/or pepper plants) and/or crops are prepared, such as through classical breeding or genetic engineering (e.g., genetically modified or transgenic plants, gene edited plants, and the like), with modified levels and compositions of SGAs, conferring on the plant a chemical barrier against a broad range of insects and other pathogens and/or removing anti-nutritional compounds (e.g., chaconine and/or solanine from potato).

Furthermore, high cholesterol or high phytosterol tomato lines are used to engineer high value steroidal compounds (e.g., pro-vitamin D and/or diosgenin), such as through synthetic biology tools.

In addition, high phytosterol (e.g., phytocholesterol) lines are used to produce components used in cosmetic products.

In other instances, Solanaceous plants (e.g., tomato, potato, eggplant, and/or pepper plants) and/or crops are prepared with increased levels of SGAs and/or decreased levels of phytosterols.

### Example 14—Materials and Methods for Examples 14-25

### Liquid Chromatography Mass Spectroscopy (LC-MS) and Tandem Mass Spectroscopy (MS/MS) Parameters for Saponin Analysis

Four biological replicates (n=4) from sample were used for metabolic analysis. Briefly, 100 mg of frozen powdered plant tissue was extracted with 300 μL of 80% methanol mixed with internal standard (Ponasterone A, C=1.5 μg/mL), briefly vortexed and then sonicated for 20 min. at room temperature. Extracts were centrifuged for 10 min. at 14,000×g and filtered through 0.22 μm filters. Samples were analyzed using a high-resolution UPLC/qTOF system comprised of a UPLC (Waters Acquity) connected to a SYNAPT-G2 qTOF detector (tandem quadrupole/time-of-flight mass spectrometer, Waters). Separation of metabolites was performed on a 100×2.1 mm i.d., 1.7 μm UPLC BEH C18 column (Waters Acquity). The mobile phase consisted of 0.1% formic acid in acetonitrile:water (5:95, v/v; phase A) and 0.1% formic acid in acetonitrile (phase B). The flow rate was 0.3 mL/min, and the column temperature was kept at 35° C.

The following linear gradient was used for analysis of triterpenoid saponins in spinach, Beta vulgaris, triterpenoids produced in yeast cells and in vitro with use of SOAP10: from 100% to 85% phase A over 5 min., from 85% to 75% phase A over 2 min., then held at 75% phase A for 3 min.; gradient continued to 65% A over 10 min., from 65% to 40% A over 2 min.; from 40% phase A to 100% phase B over 1 min., then held at 100% phase B 3.5 minutes and then returned to the initial conditions (100% phase A) within 0.5 min. and conditioning at 100% phase A for 1 min.

To analyze triterpenoid saponins from Chenopodium quinoa and Medicago sativa 40 min. gradient was used: from 100% to 72% phase A over 22 min., from 72% to 0% phase A over 14 min., then held at 100% phase B for 2 min.; and then returned to the initial conditions (100% phase A) within 0.5 min. and conditioning at 100% phase A for 1.5 min.

The following settings were used: capillary 2 kV; cone 27 V; source temperature was set to 140° C., desolvation 450° C., desolvation gas flow 800 L/h. Argon was used as the collision gas. Electrospray ionization (ESI) was used in negative ionization mode at the m z range of 50-1600 Da. The mass spectroscopy (MS) system was calibrated using sodium formate, and Leu-enkephalin was used as the lock mass. MassLynx software version 4.1 (Waters) was used to control the instrument and calculate accurate masses and elemental compositions. In addition, a mixture of 15 standard metabolites, injected after each of the 10 samples, was used as quality controls. Data acquisition was performed in the MS^{E} mode with energy ramp that records an exact mass precursor and fragment ion information from every detectable component in a sample. MS^{E} mode rapidly alternates between two functions: the first acquiring low-energy exact mass precursor ion spectra and the second acquiring elevated energy exact mass fragment ion spectra. The collision energy for low-energy function was set to 4 eV, and for the high-energy to 15-50 eV ramp.

For separation of the products of the Glycyrrhiza uralensis enzymes in N. benthamiana another gradient was used: starting at 75% phase A for 5 min., then from 75% to 50% phase A over 15 min., from 50% to 30% phase A over 2 min., from 30% phase A to 100% phase B, then held at 100% phase B for 3.5 min. and then returned to the initial conditions (75% phase A) within 0.5 min. and conditioning at 75% phase A for 1 min. The flow rate was 0.3 mL/min., and the column temperature was kept at 35° C. Samples were analyzed using a high-resolution UPLC/qTOF system comprised of a UPLC (1290 Infinity II, Agilent) connected to an Impact HD UHR-QqTOF (Bruker). Separation of metabolites was performed on a 100×2.1 mm i.d., 1.7 μm UPLC BEH C18 column (Waters Acquity). The mobile phase consisted of 0.1% formic acid in acetonitrile:water (5:95, v/v; phase A) and 0.1% formic acid in acetonitrile (phase B). ESI was used in negative ionization mode at the m z range of 50-1700 Da with following parameters: drying gas: 200° C., 8 L/min; nebulizer: 2 Bar; capillary: 4.2 kV.

Metabolites were identified by comparing the retention times and mass fragments of standard compounds. When corresponding standards were not available, compounds were putatively identified by comparing their retention times, elemental composition and fragmentation pattern with those described in the literature (Table 14). Relative quantification of triterpenoid saponins in spinach was carried out using the TargetLynx (Waters) program. Peak area of saponins in each sample was normalized to peak area of internal standard in order to reduce variability originating from sample handling.

### Gas Chromatograph Mass Spectroscopy (GC-MS) Analysis of Spinach Triterpenoid Aglycones

Powdered frozen tissue (200 mg) was extracted 3 times with 600 μL of 80% MeOH. Collected fractions were evaporated in speed-vac (25° C., 78 mbar, O/N). Dry residue was dissolved in 500 μL of 2M HCl in 50% MeOH and heated for 5 hours at 65° C. Hydrolyzed samples were evaporated to dryness in the speed-vac (50° C., 78 mbar, 1.5h). Dry residue was dissolved in 200 μL of Toluene:MeOH (3:2; v/v) and 75 μL of trimethylsilyldiazomethane (TMSCHN₂) were added. Methylation mixture was incubated at room temperature for 40 minutes, evaporated to dryness and resuspended in 80 μL of MSTFA, incubated for 20 min. at room temperature and 10 minutes at 65° C. Sample was transferred to the glass insert and 1 μL was injected onto GC-MS as described before (31). The GC-MS system comprised a COMBI PAL autosampler (CTC Analytics), a trace GC ultra-gas chromatograph equipped with a programmable temperature vaporizing (PTV) injector, and a DSQ quadrupole mass spectrometer (Thermo Electron). GC was performed on a 30 m×0.25 mm×0.25 m Zebron ZB-5 ms MS column (Phenomenex). The PTV split technique was performed as follows: Samples were analyzed in the constant temperature splitless mode. PTV inlet temperature was set at 280° C. Analytes were separated using the following chromatographic conditions: Helium was used as carrier gas at a flow rate of 1.2 mL/min. The thermal gradient started at 170° C., was held at this temperature for 1.5 min, ramped to 280° C. at 37° C./min. and then ramped to 300° C. at 1.5° C./min. and held at 300° C. for 5.0 min. Eluents were fragmented in the electron impact mode with an ionization voltage of 70 eV. The reconstructed ion chromatograms and mass spectra were evaluated using Xcalibur software (ThermoFinnigan). Compounds were identified by comparison of their retention index and mass spectrum to those generated for authentic standards analyzed on the same instrument and those reported in literature (E. Biazzi et al., CYP72A67 Catalyzes a Key Oxidative Step in Medicago truncatula Hemolytic Saponin Biosynthesis. Molecular Plant. 8, 1493-1506 (2015)).

### Saponin Purification for NAMR Analysis

The system consisted of an Agilent 1290 Infinity II UPLC system equipped with a quaternary pump, auto sampler, diode array detector, a Bruker/Spark Prospekt II LC-SPE system (Spark) and Impact HD UHR-QqTOF mass spectrometer (Bruker) connected via a Bruker NMR MS Interface (BNMI-HP) as described previously (B. Khakimov, L. H. Tseng, M. Godejohann, S. Bak, S. B. Engelsen, Screening for Triterpenoid Saponins in Plants Using Hyphenated Analytical Platforms. Molecules. 21, pii: E1614 (2016)). MS spectra, in negative mode, were acquired between m z 50 and 1700. The calibration was done with a 10 mM sodium trifluoroacetate (NaTFA, Sigma-Aldrich) automatically which is introduced at the beginning and the end of each chromatographic run. Separation was done on a XBridge LC column (BEH C18, 5 m particle size, and 250 mm×4.6 mm; Waters). The chromatographic conditions were as following; a flow rate of 0.9 mL/min. starting with a solvent composition of 74% A (5% ACN+0.1% FA) and 26% B (100% ACN+0.1% FA) with a linear gradient to 70% A at 60 min., followed by another linear gradient to 100% B at 62 min. 100% B hold for 3 min. followed linear gradient to 74% A at 65.5 and hold for 4.5 min. for equilibration. Saponins were collected on SPE cartridges in preset time windows and trapping was triggered by intensity threshold. Yossoside IV (m z 1263.58, threshold 200′000, time window 18-23 min.); Yossoside XII (m z 1233.57, threshold 58′000, time window 27-32 min.), Yossoside Va (m z 1305.59, threshold 200′000, time window 32-40 min.); Yossoside X (m z 1437.64, threshold 150′000, time window 40-48 min.) and Yossoside V (m z 1305.59, threshold 200′000, time window 54-65 min.). For this trapping process, a makeup-flow of 2.5 mL/min. water was added to the eluent before it passed through the SPE cartridges in order to increase the retention of analytes on the cartridges. For the trapping 10 mm×2 mm SPE cartridges filled with GP resin were used. Each cartridge was loaded five times with the same compounds, 5-10 cartridges were used for trapping one saponin. Prior to NMR measurements, SPE cartridges were dried with a stream of nitrogen, the fraction from each cartridge was eluted witha a total of 150 μL deuterated methanol. The sample was eluted into 96 well plate. Eluents containing same compound were pooled, dried under stream of nitrogen, freeze-dried, resuspended in 200 μL of D20 and freeze-dried again to remove traces of H₂O. Dry compounds were dissolved in 60 μL 90% MeOD-d4 10% D₂O with 0.01% addition of 3-propionic-2,2,3,3-d4 acid sodium salt (TMSP, that was used as an internal chemical shift reference for ¹H and ¹³C spectra) and transferred to a 1.7 mm NMR test tube.

### NMR Methods

NMR spectra were recorded on a Bruker AVANCE NEO-600 NMR spectrometer equipped with a 1.7 mm TXI-z and a 5 mm TCI-xyz CryoProbes equipped with shielded gradient coils. All spectra were acquired at 293 K. NMR data of Yossoside V were recorded on both the 1.7 and 5 mm CryoProbes, and spectra of medicagenic acid 3-O-glucuronide (MA-3-GlcA) were acquired on the 5 mm CryoProbe (using a special NMR spinner turbine to hold the 1.7 mm test tube in a 5 mm probe).

¹H and ¹³C chemical shift assignment was based on different 1D and 2D NMR techniques, all using pulsed field gradient selection.

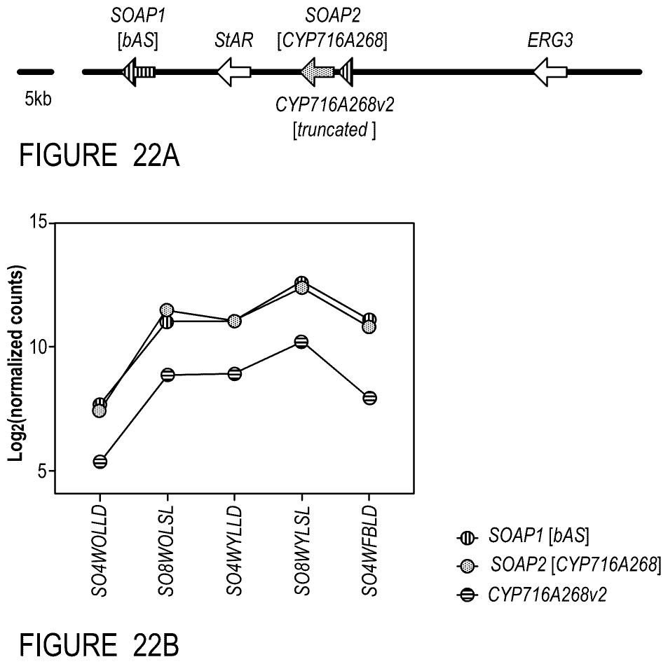

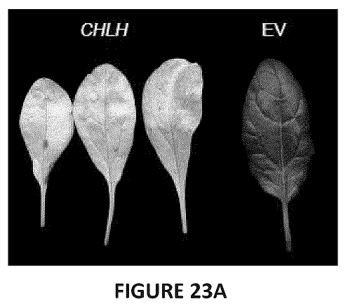

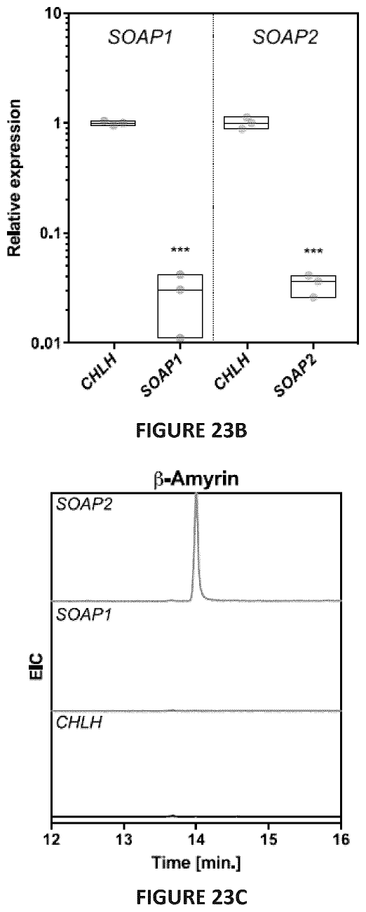

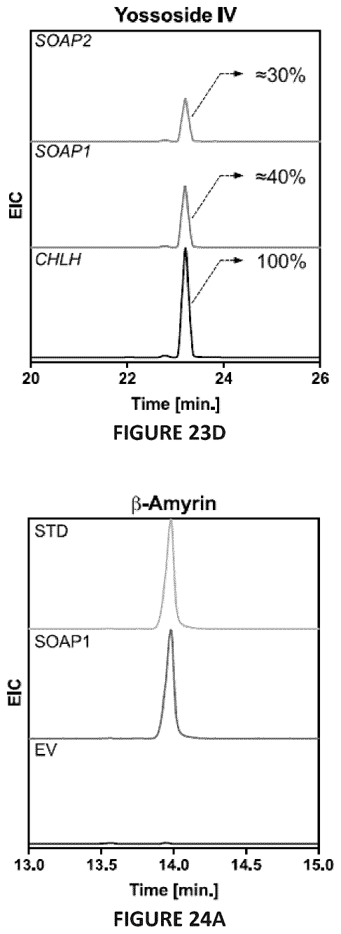

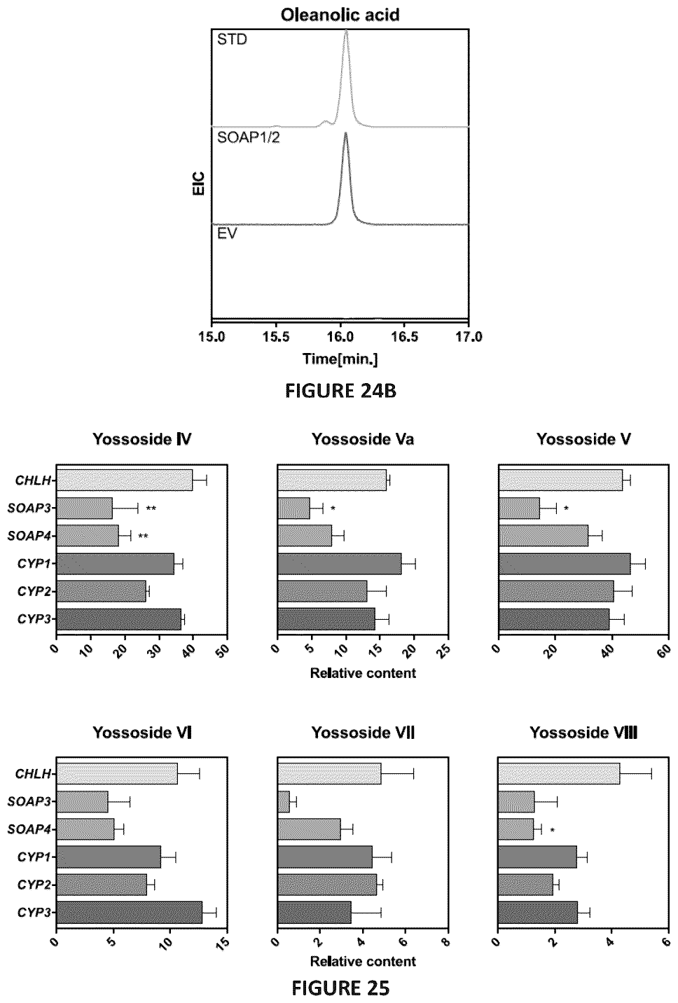

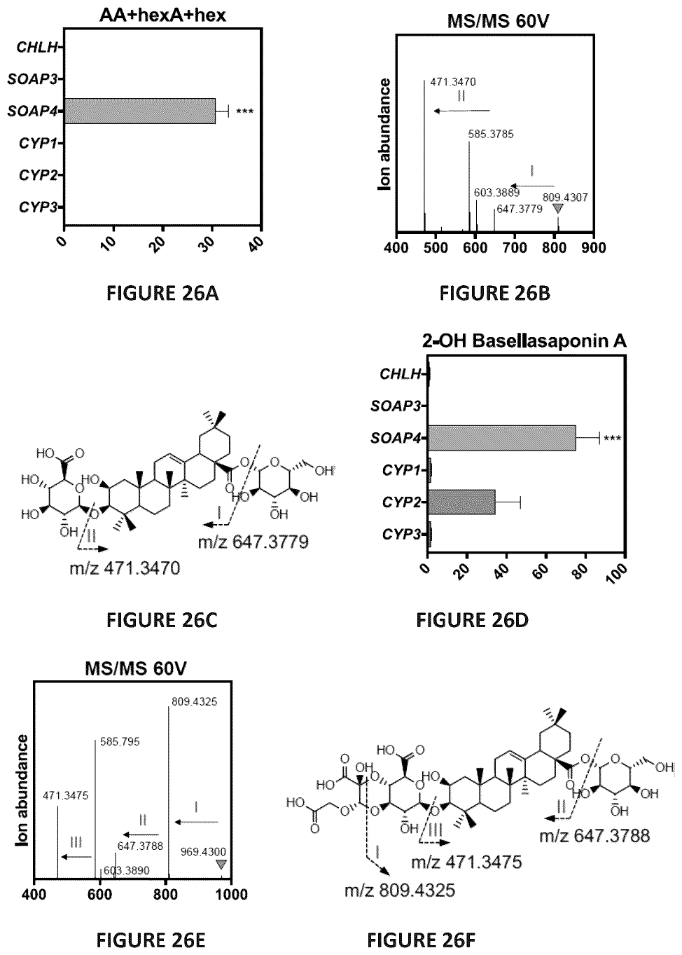

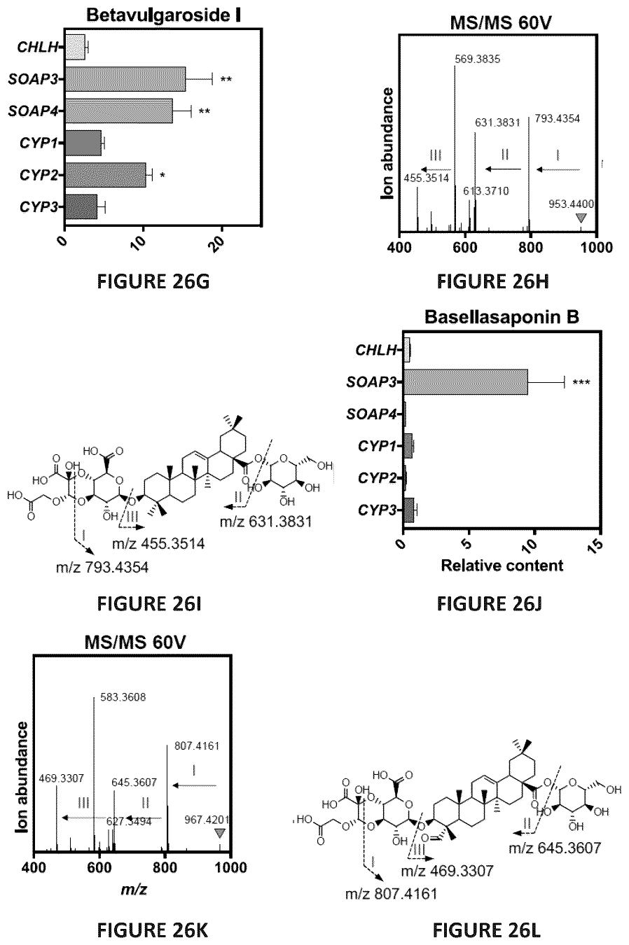

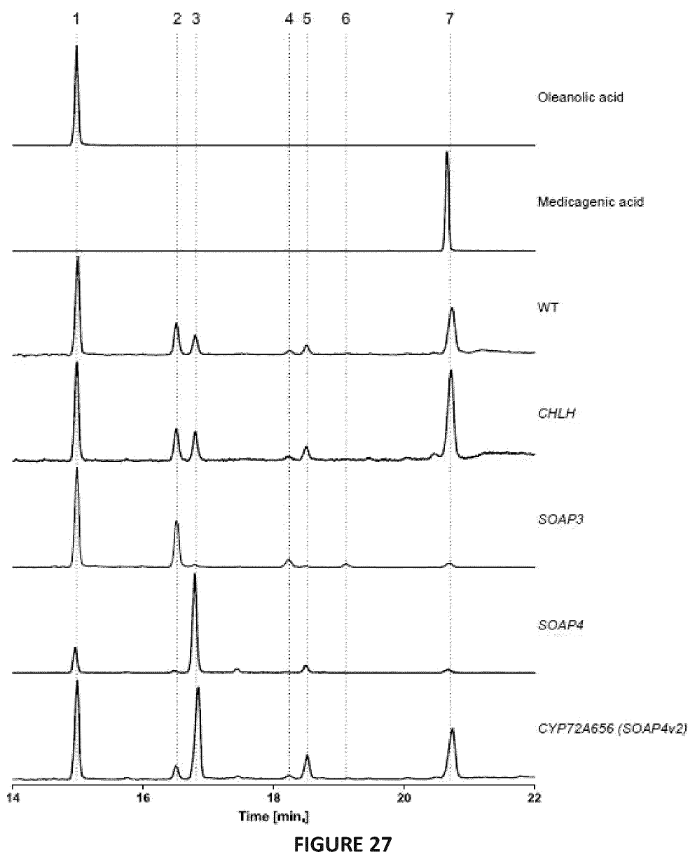

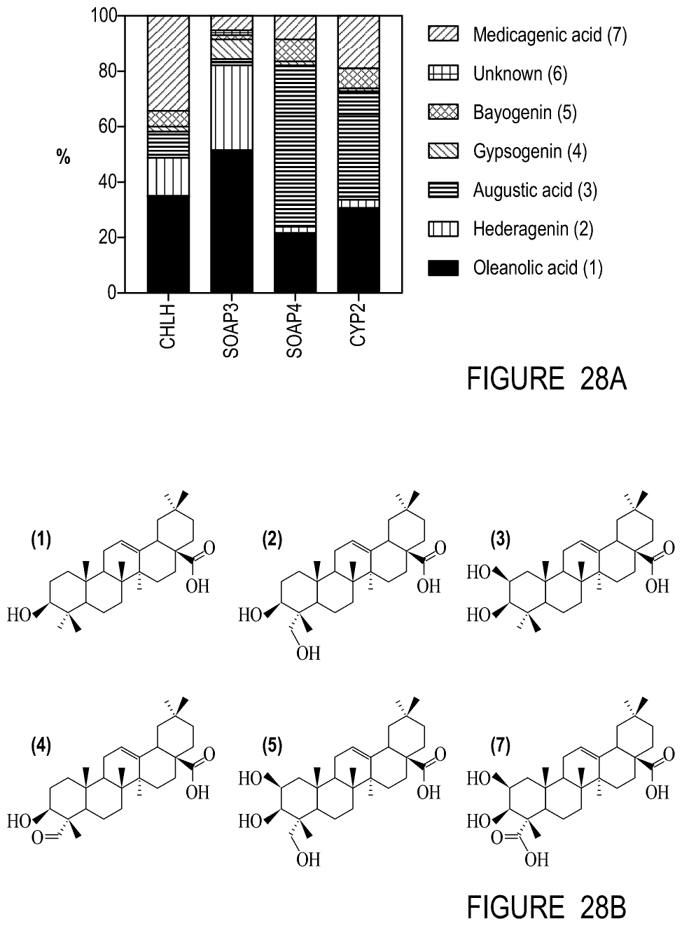

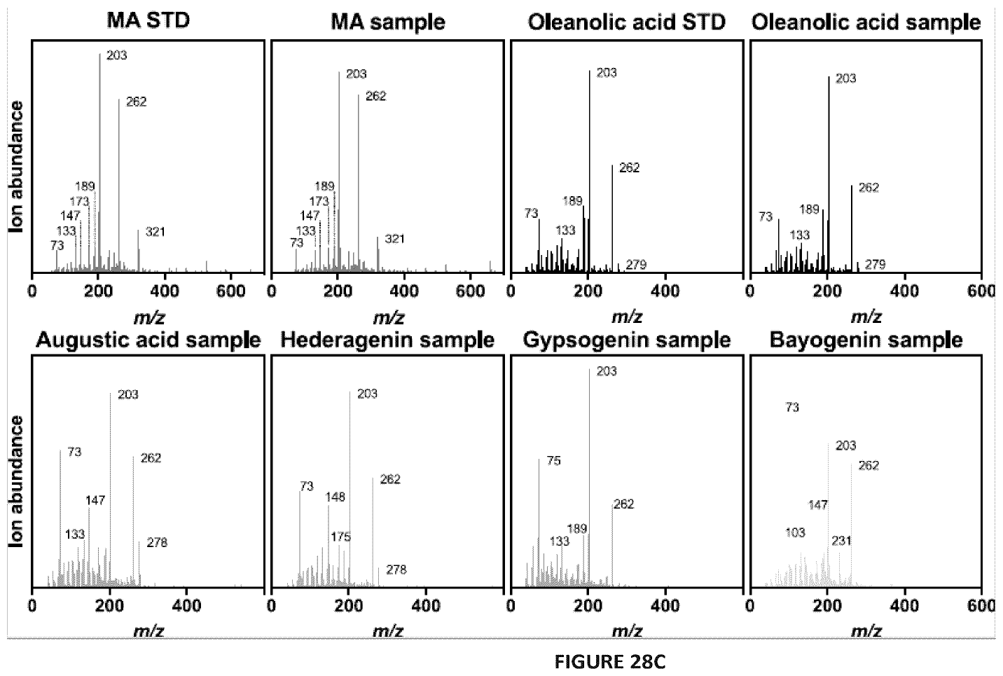

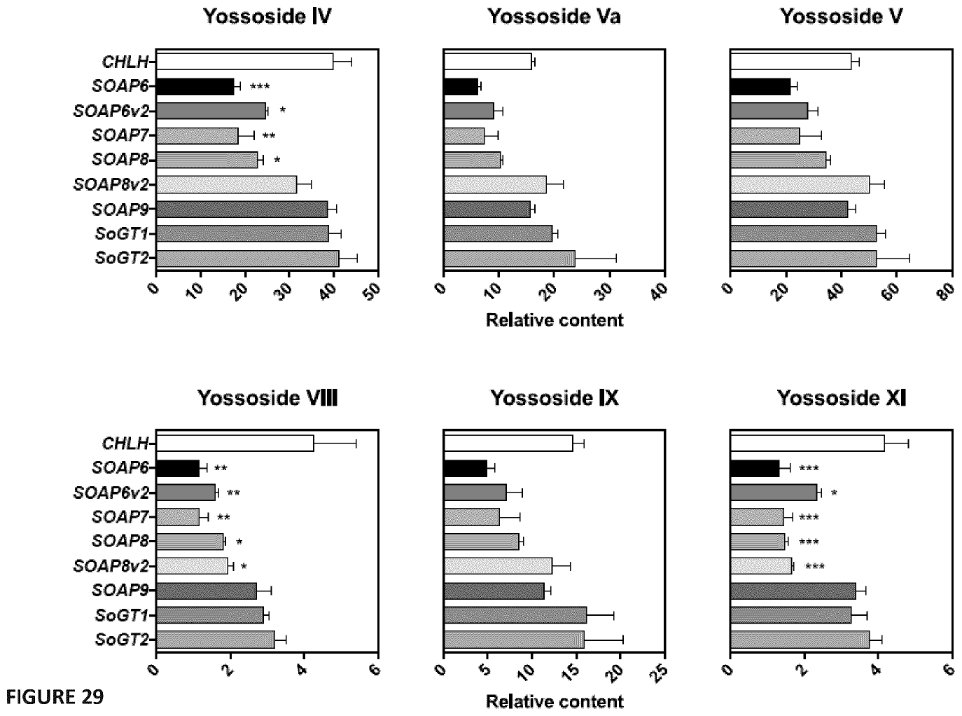

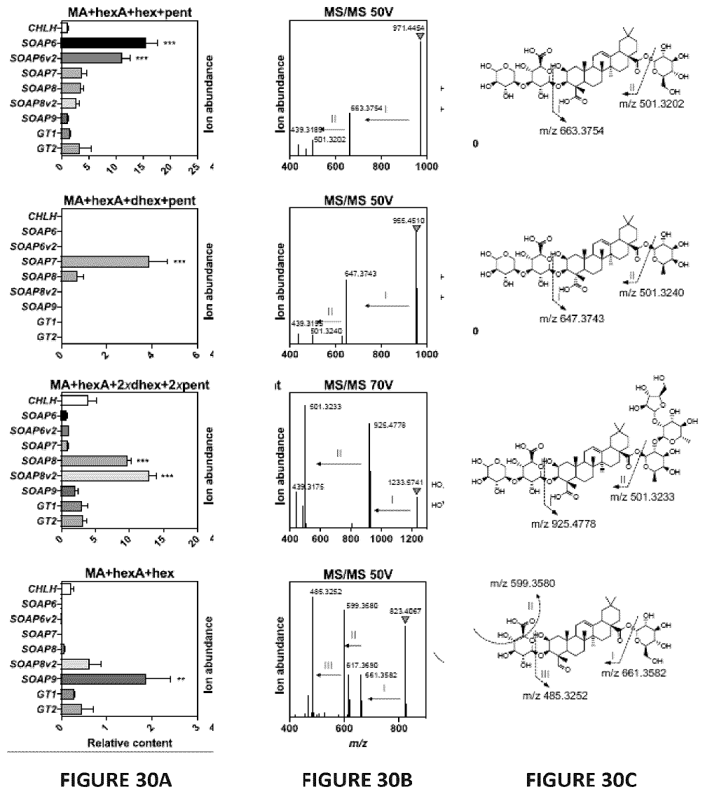

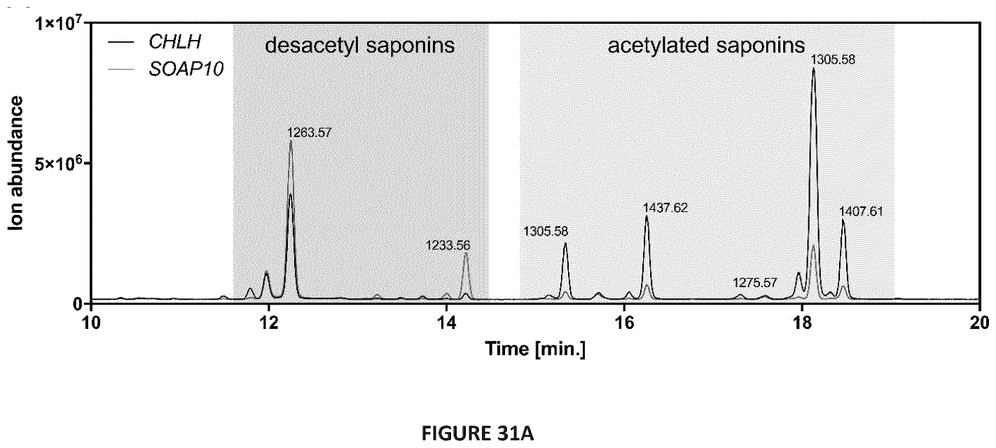

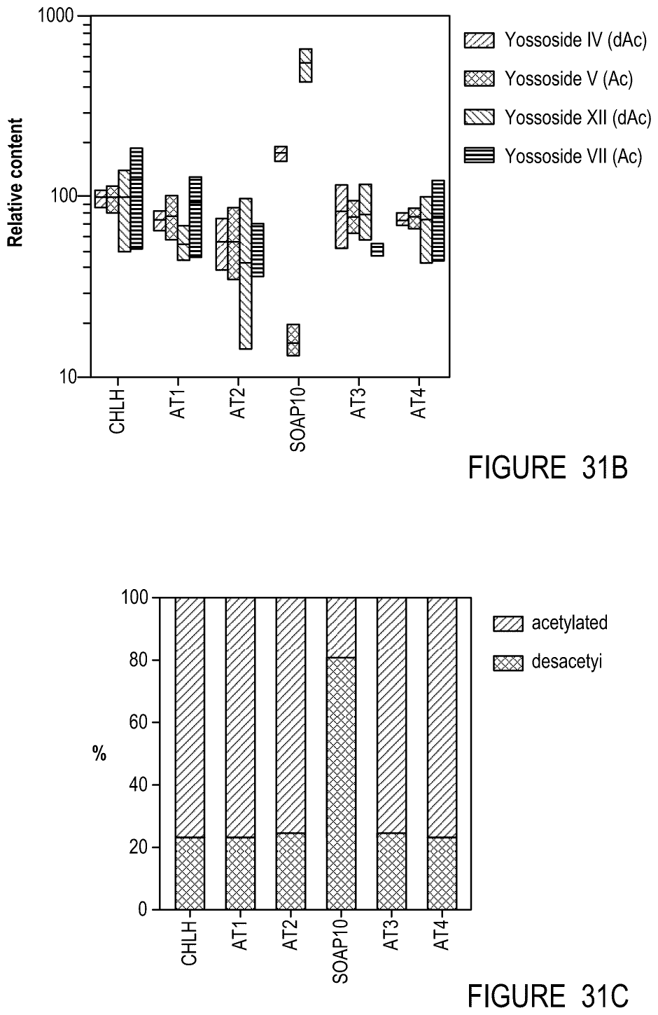

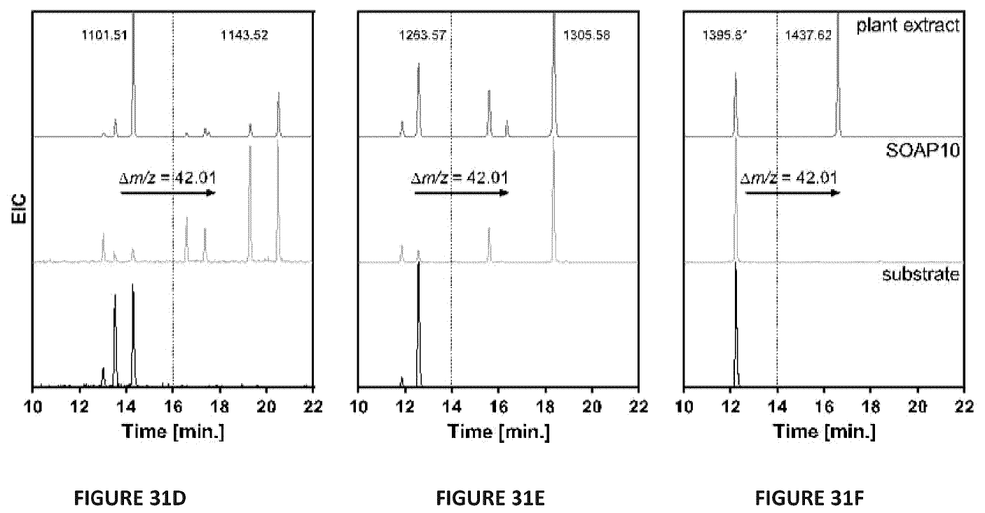

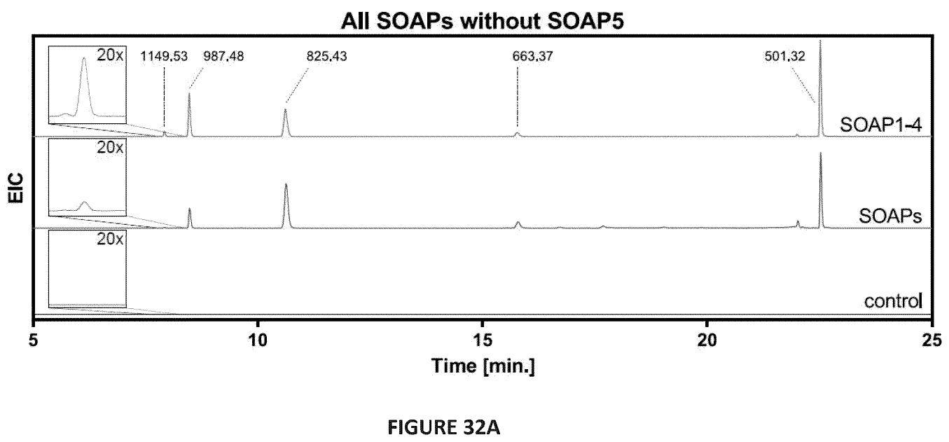

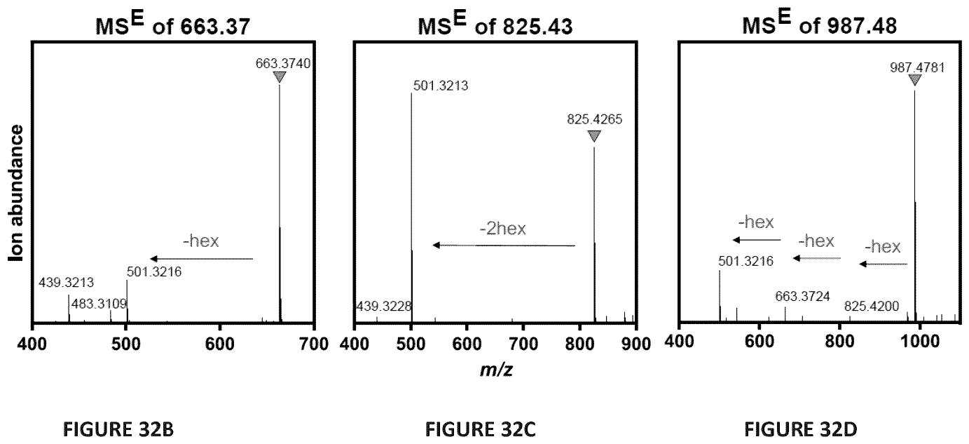

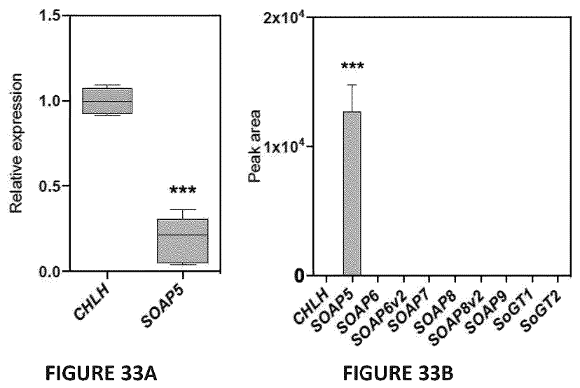

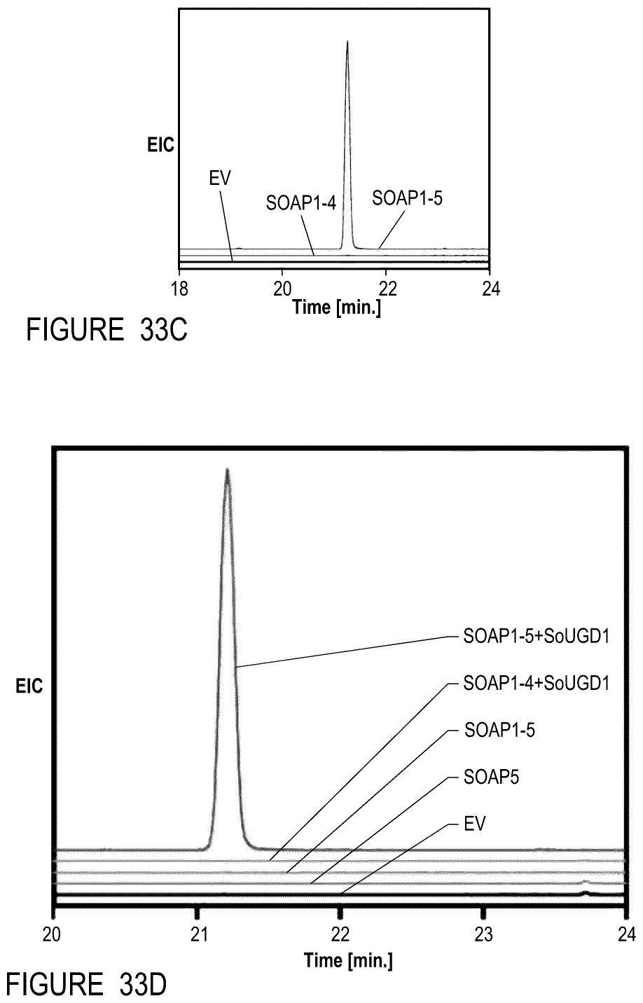

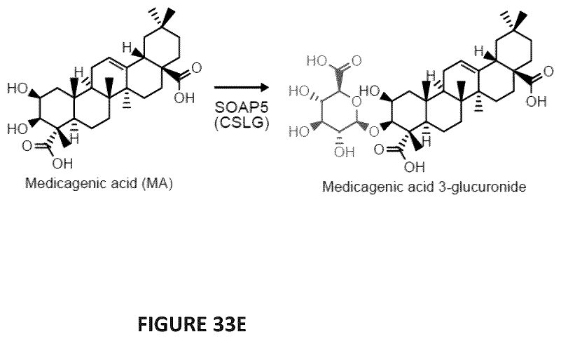

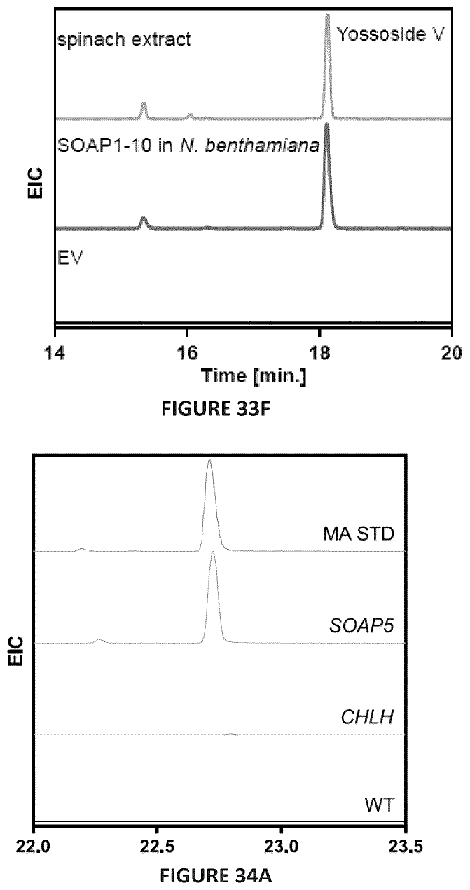

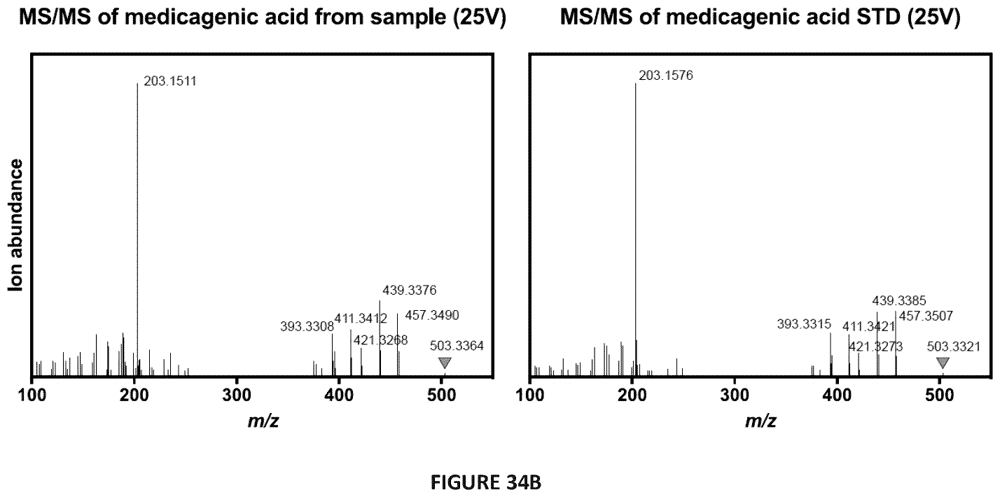

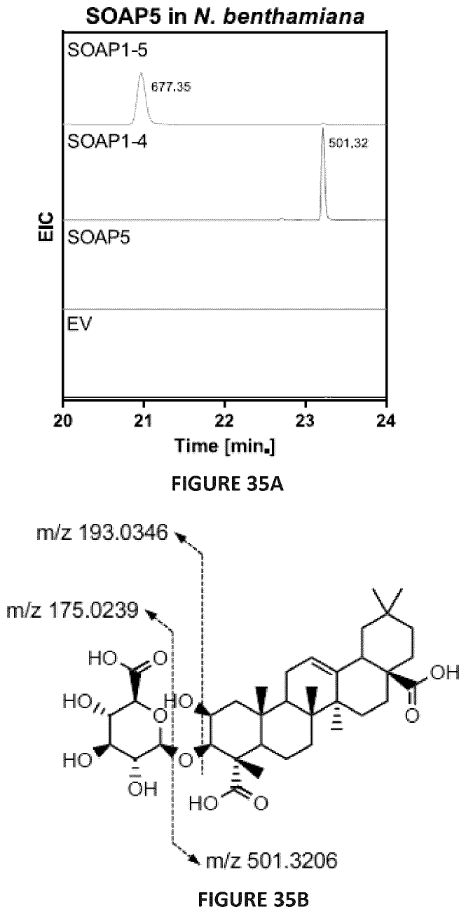

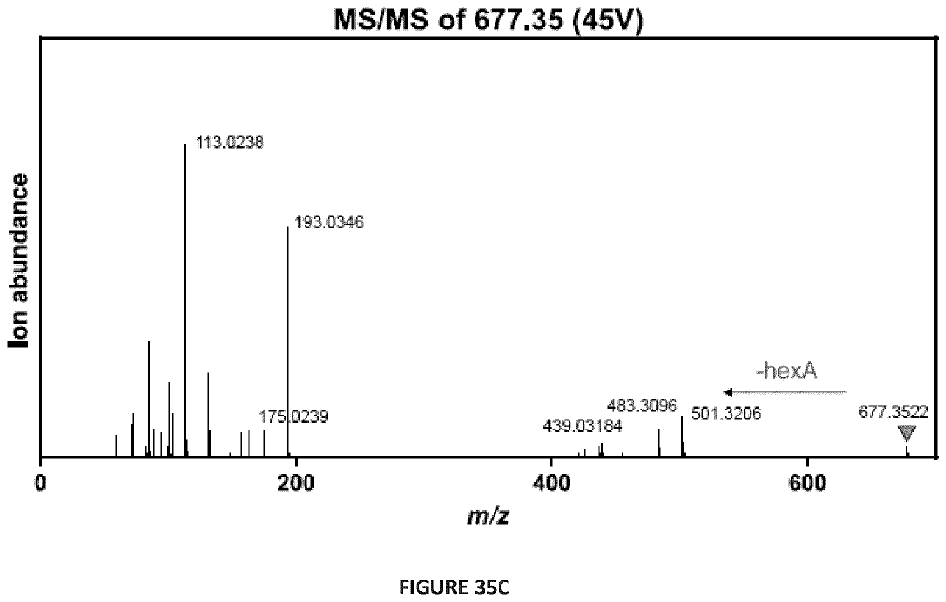

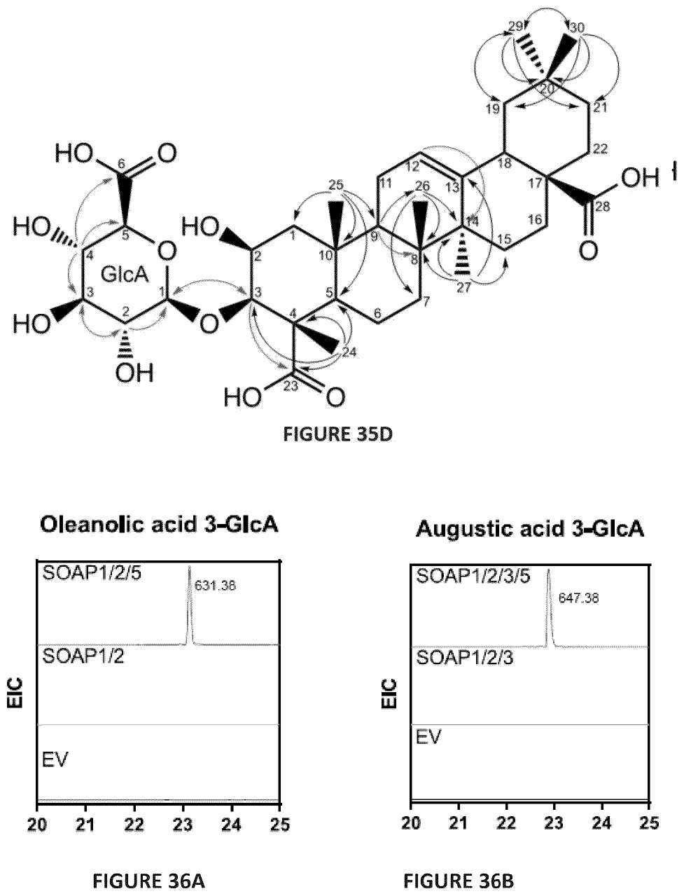

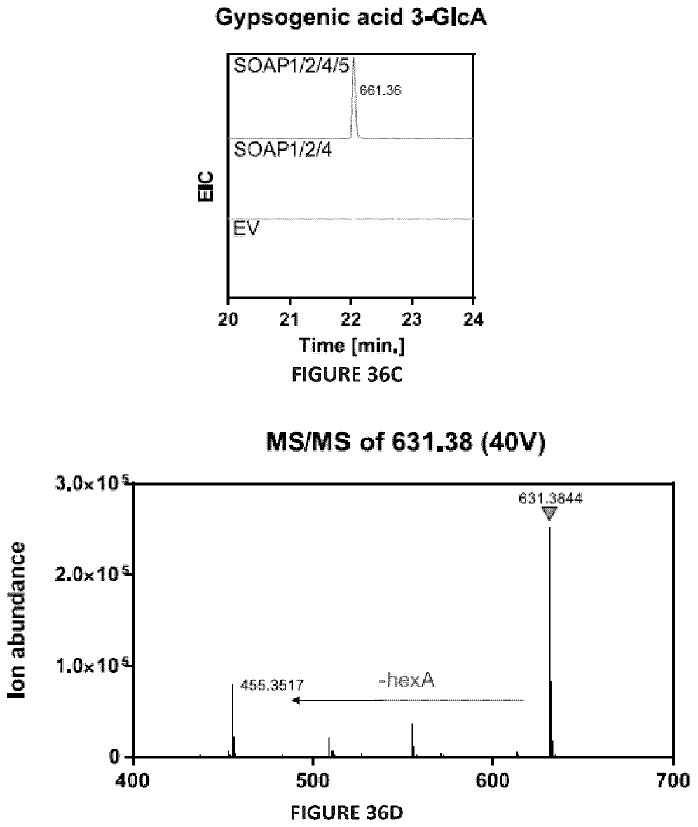

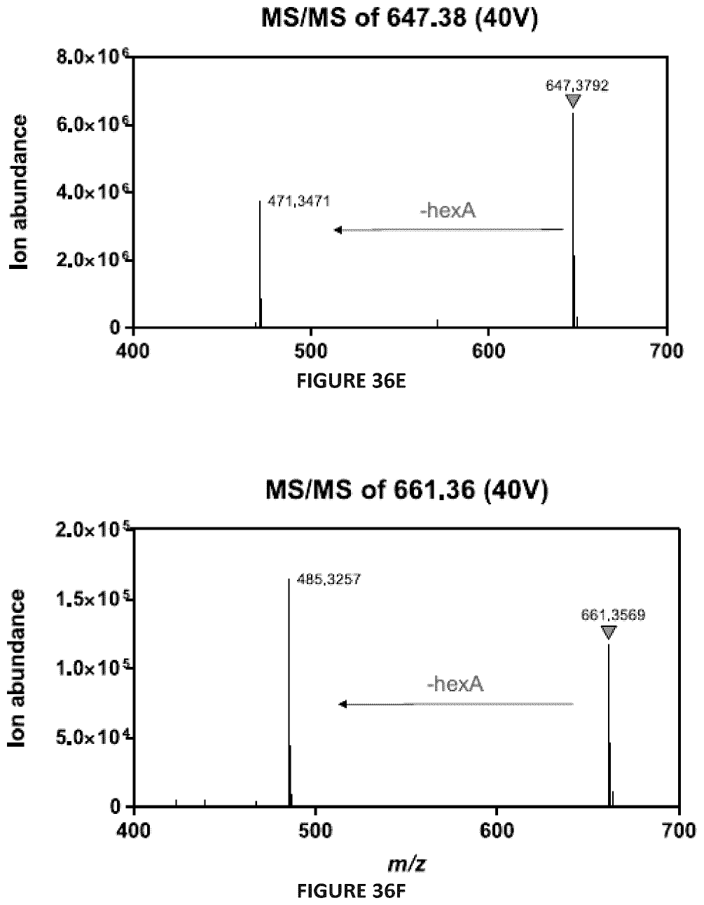

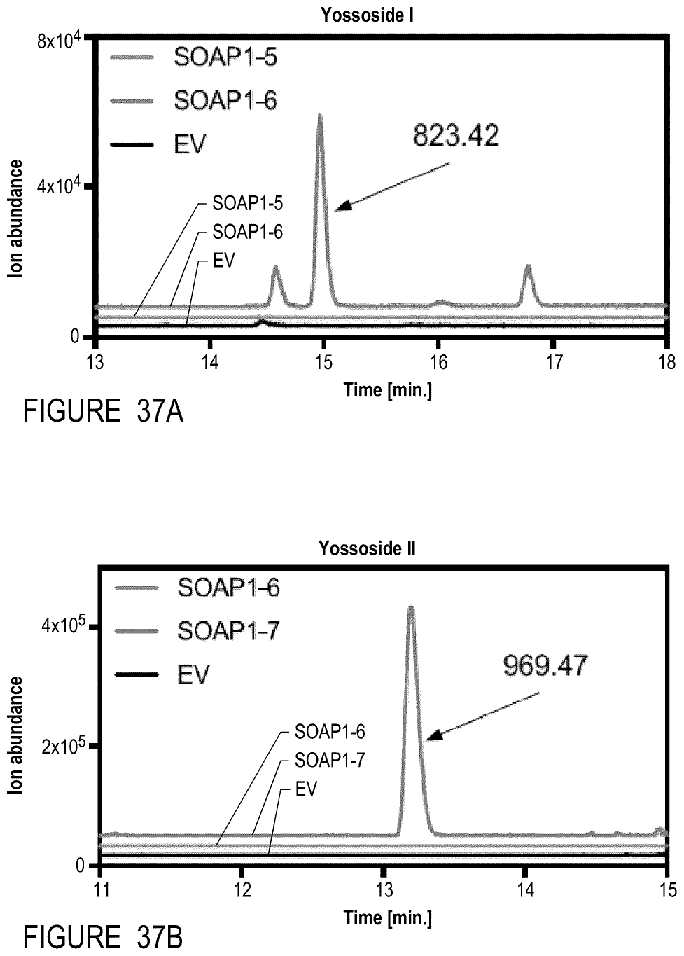

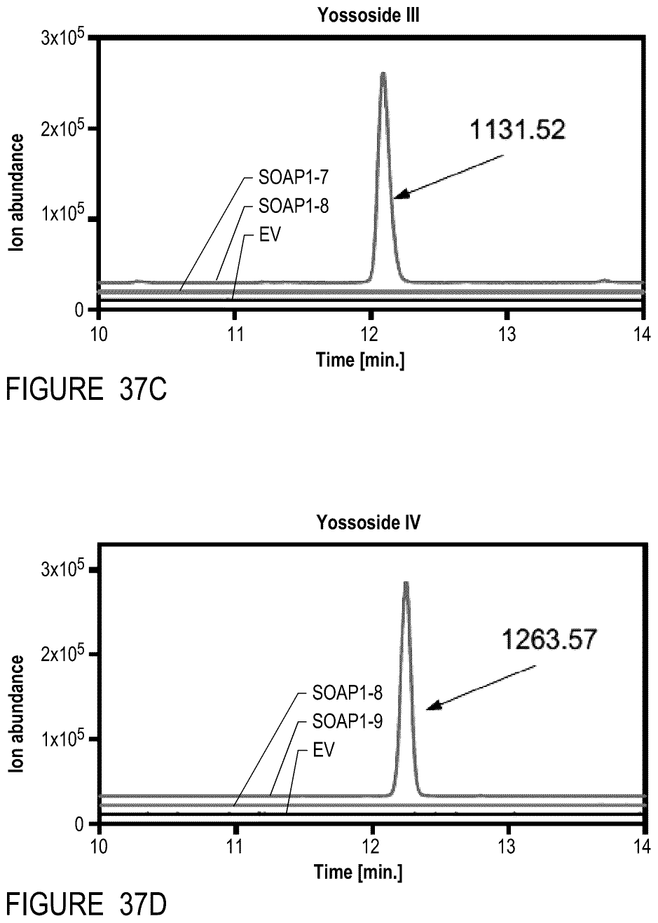

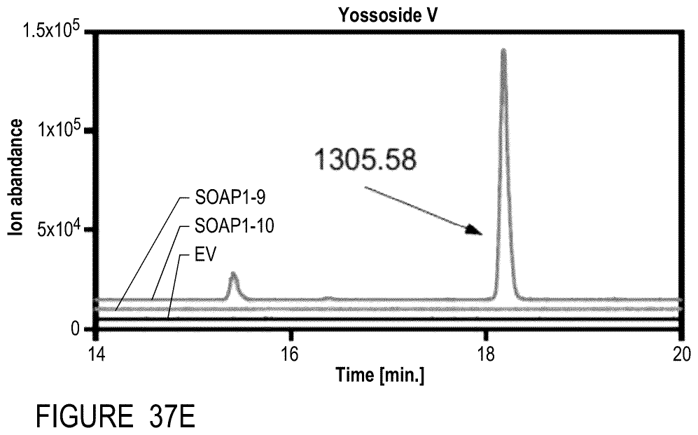

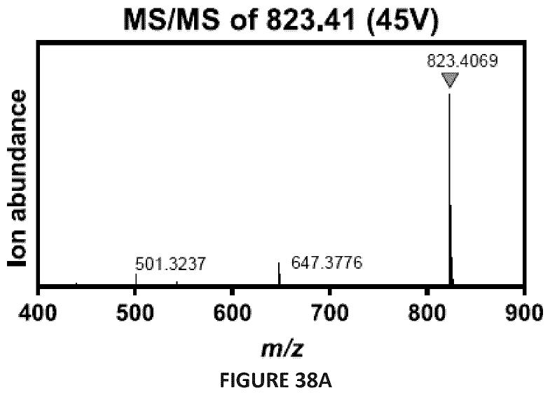

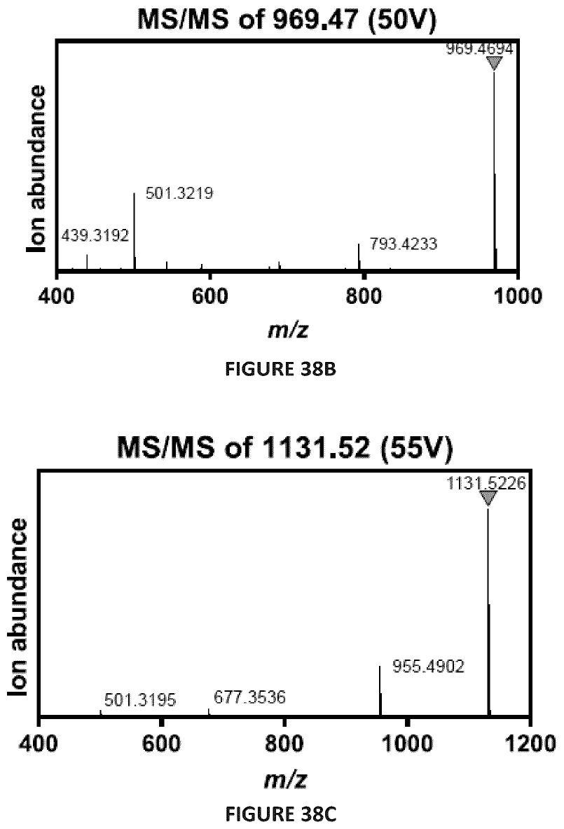

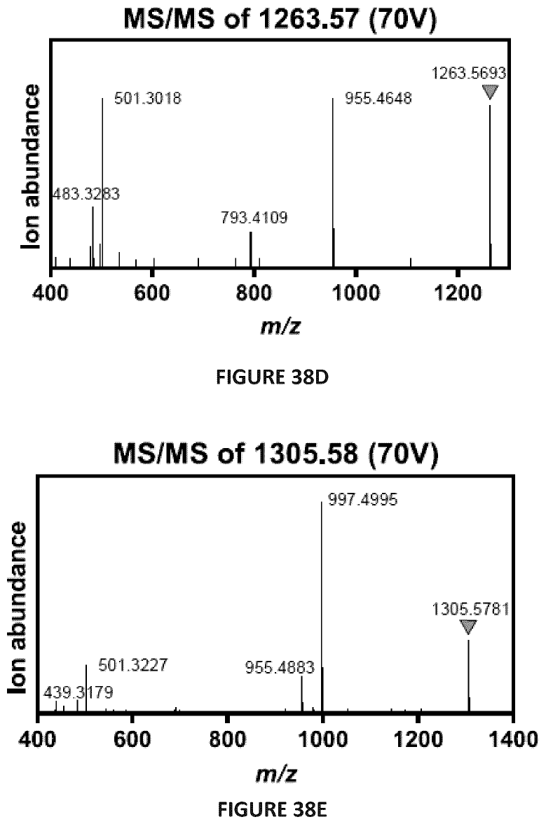

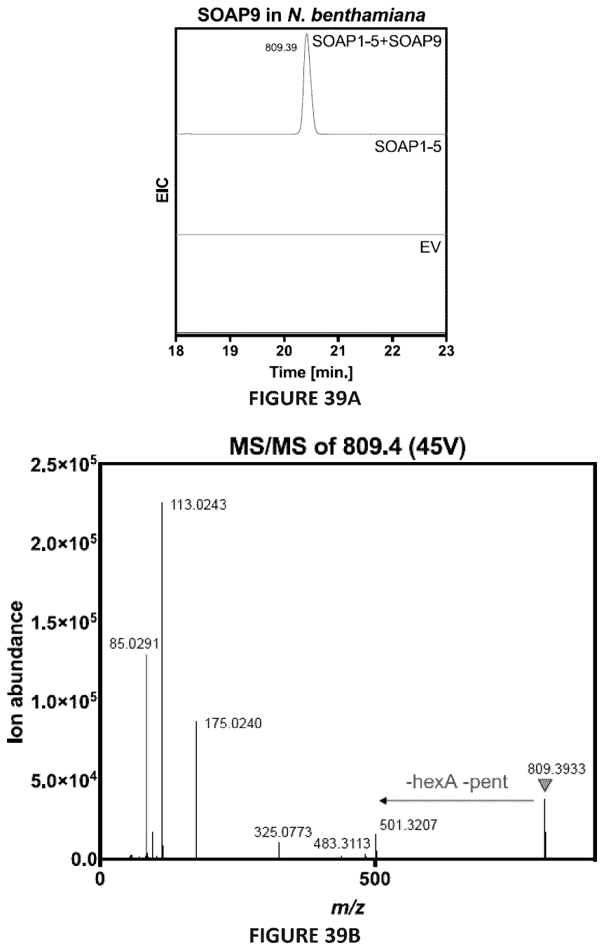

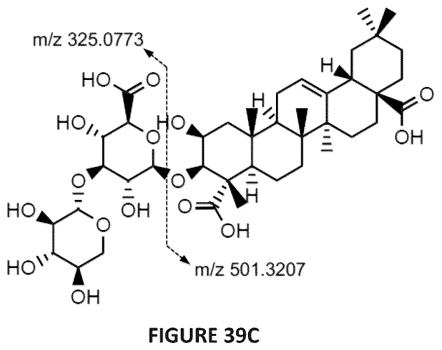

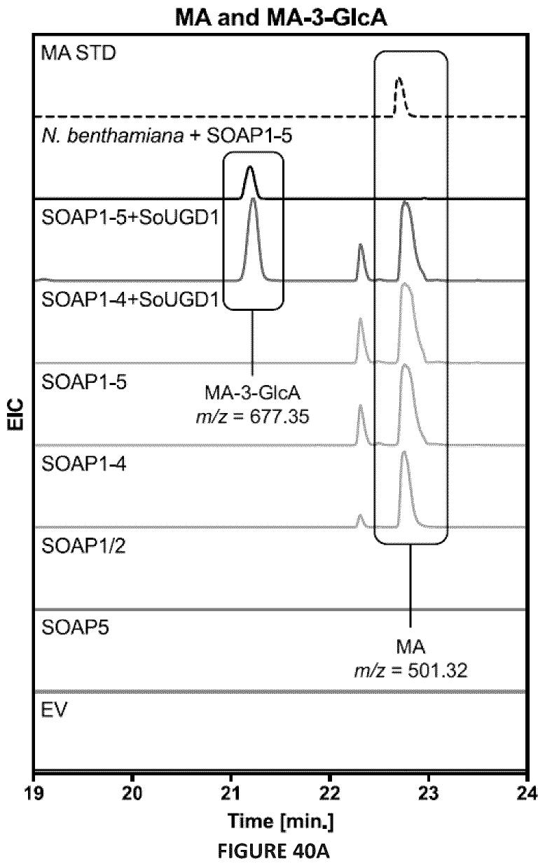

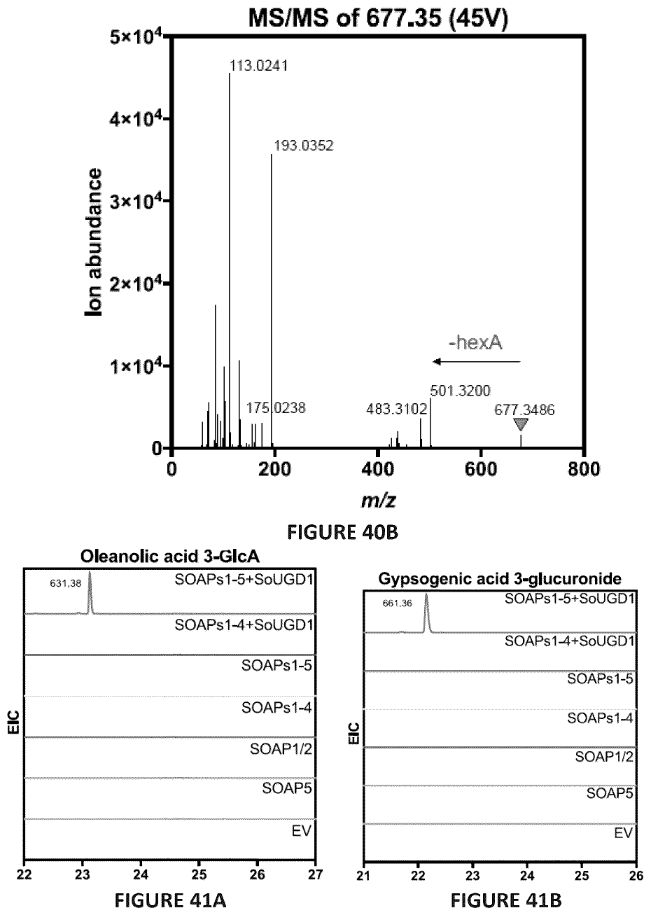

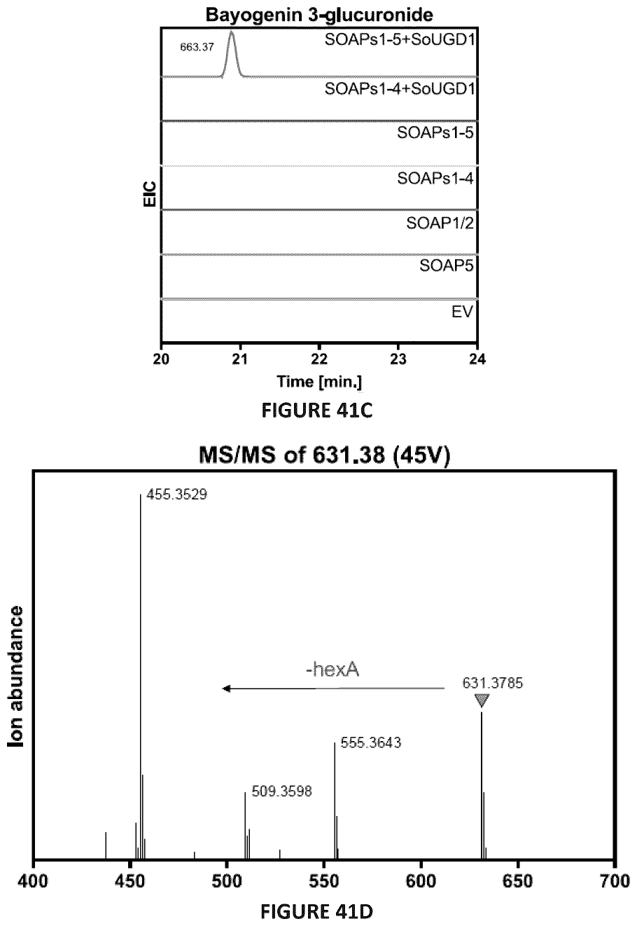

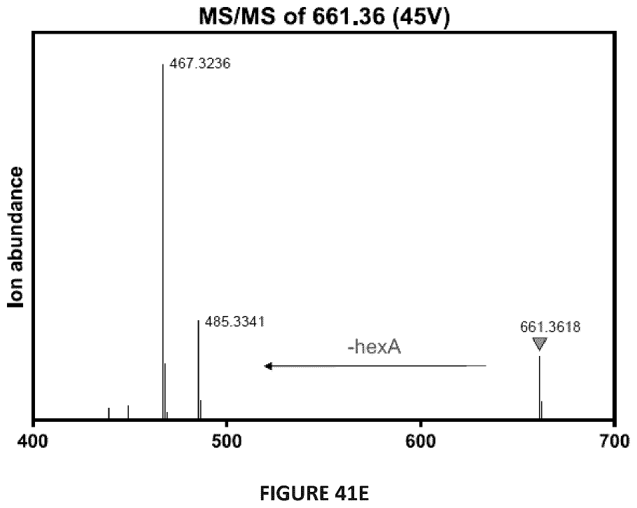

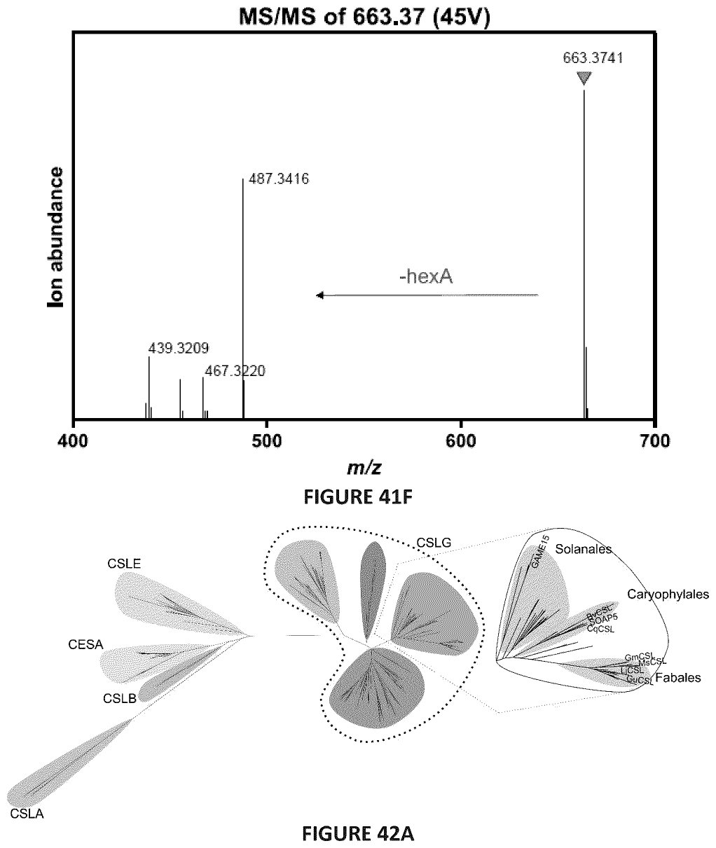

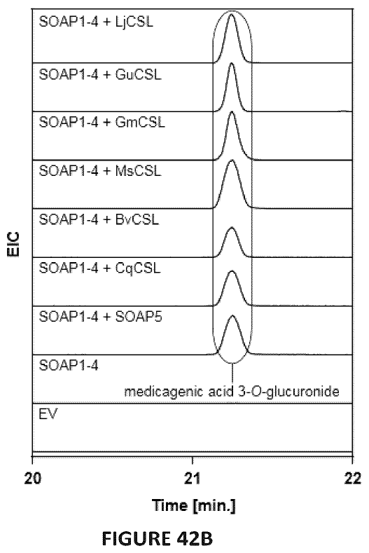

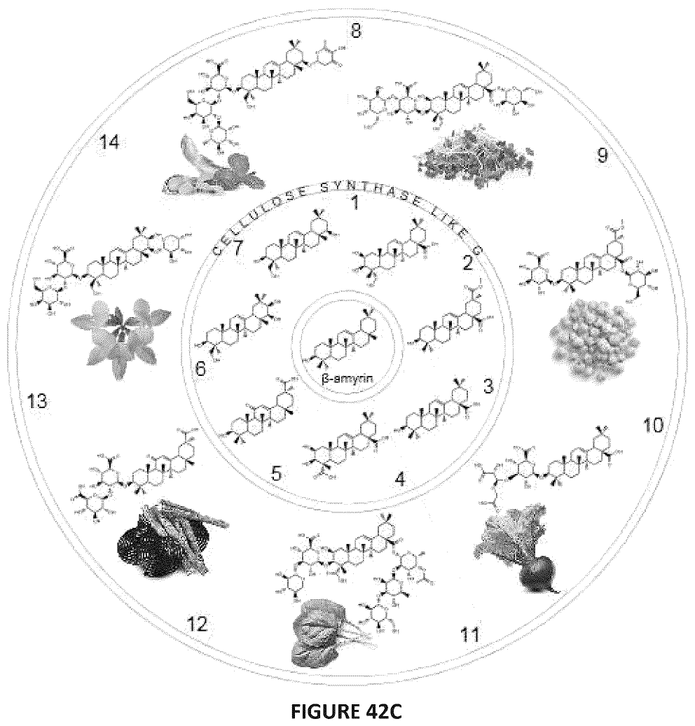

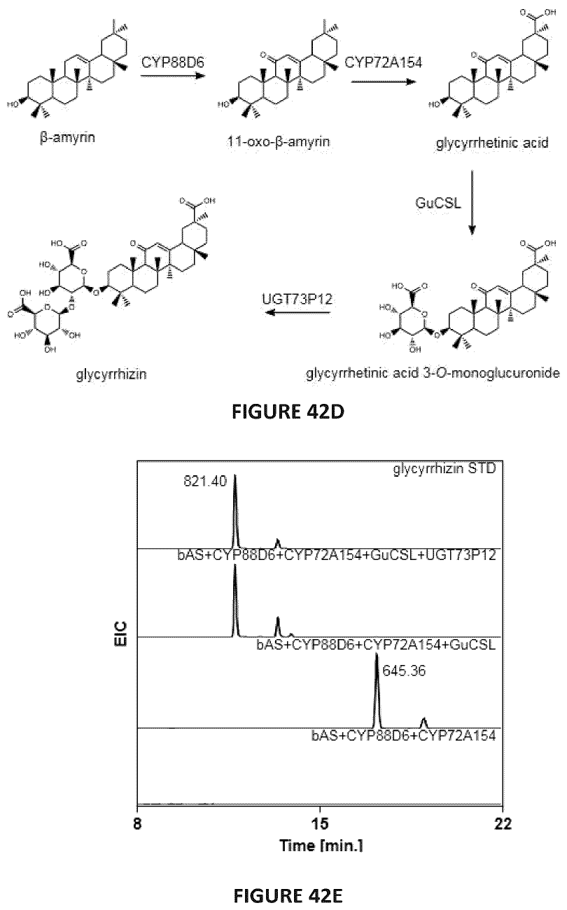

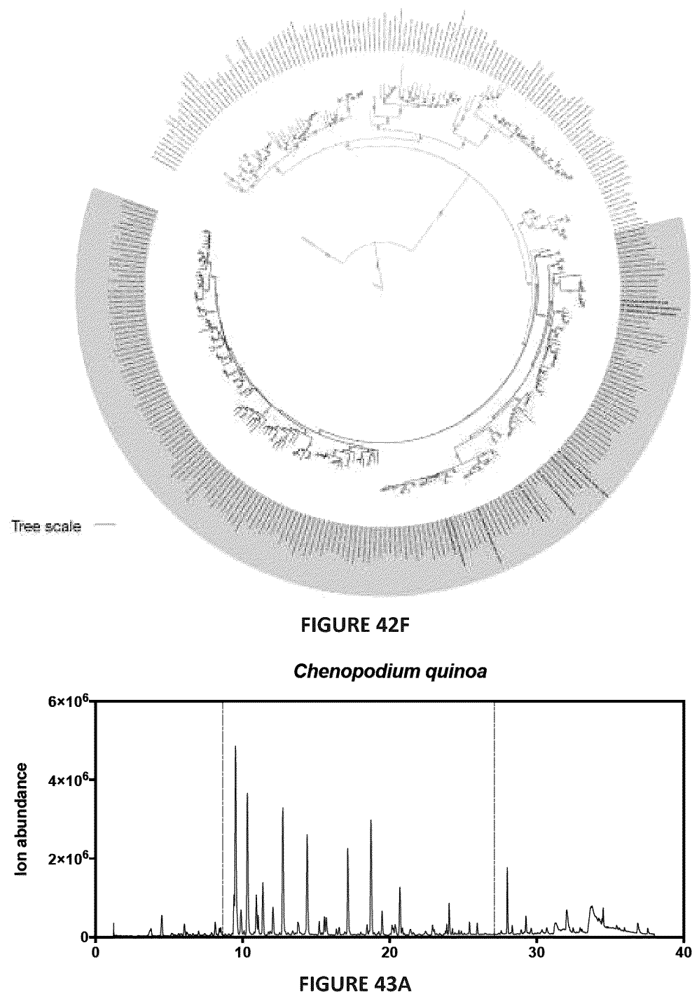

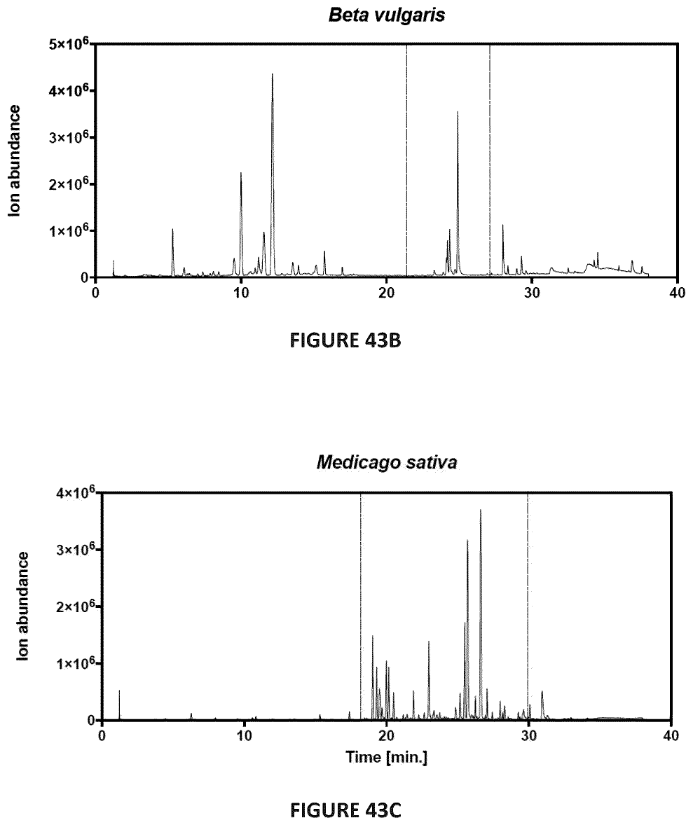

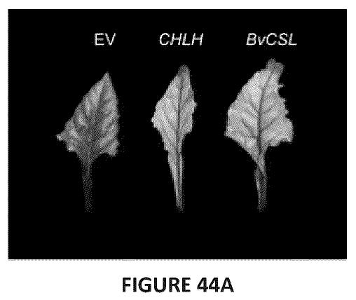

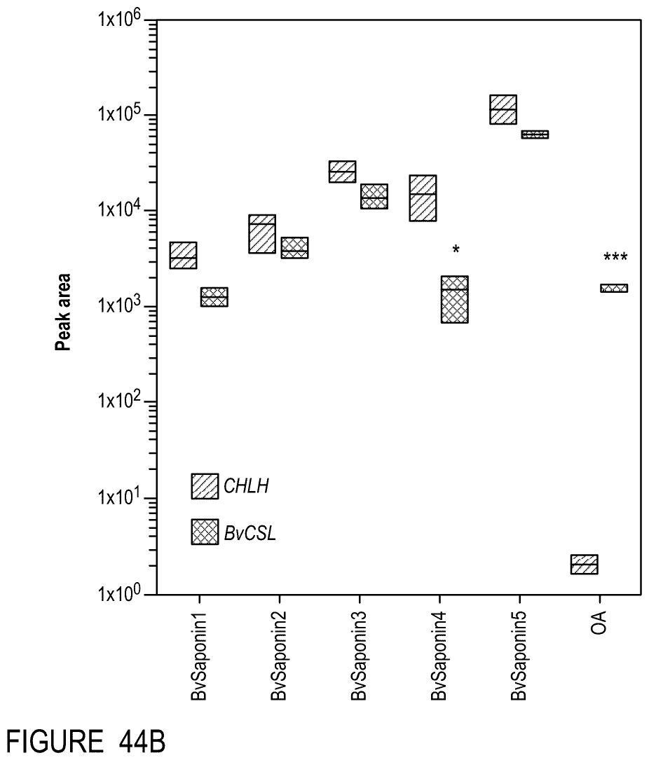

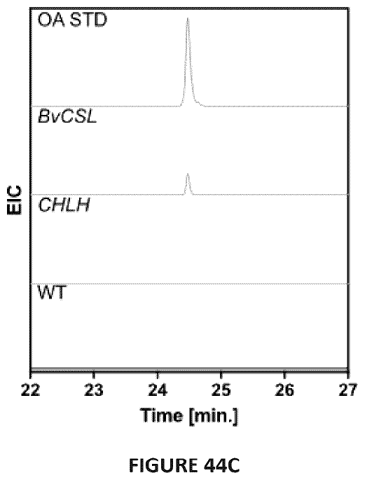

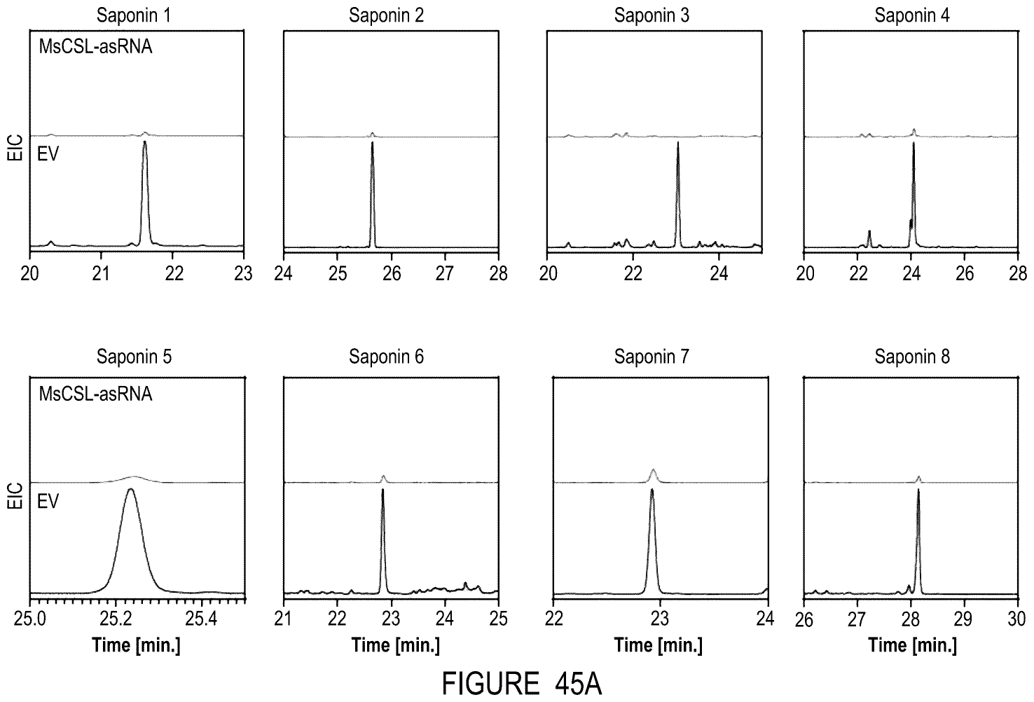

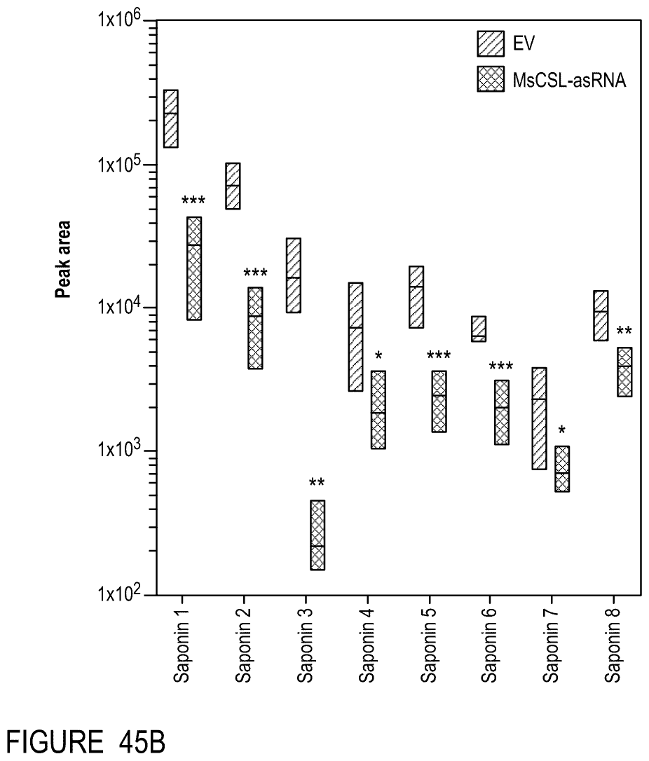

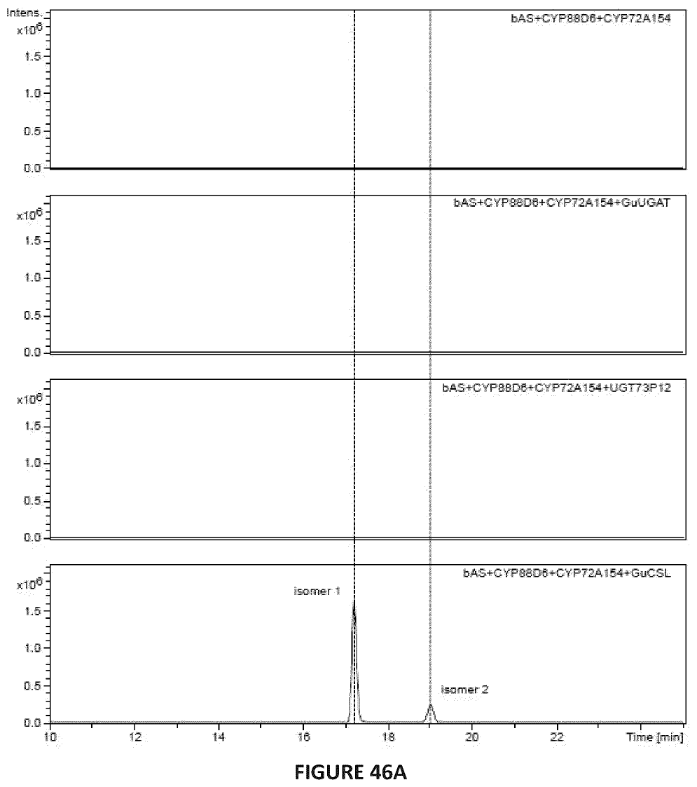

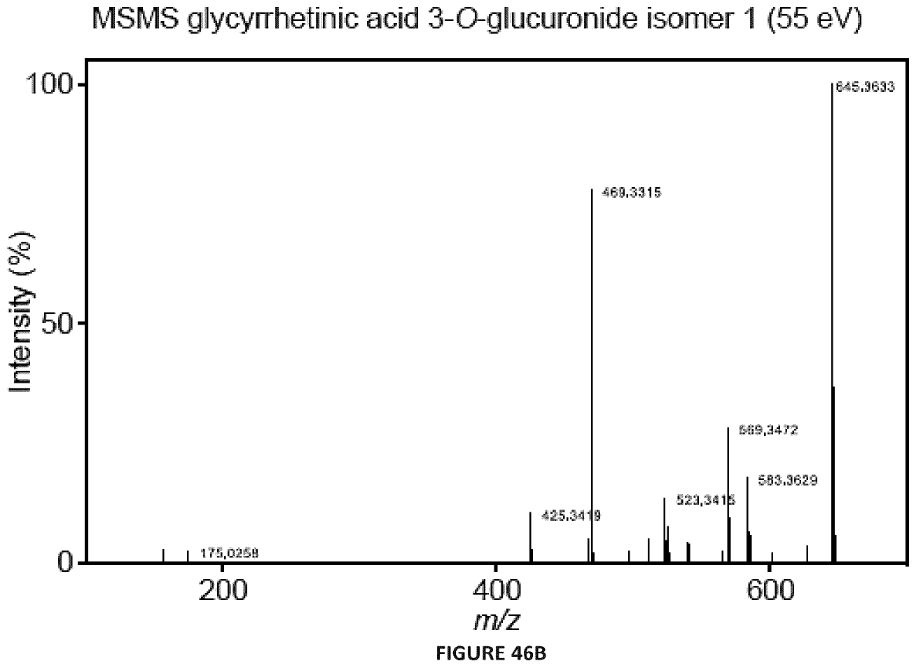

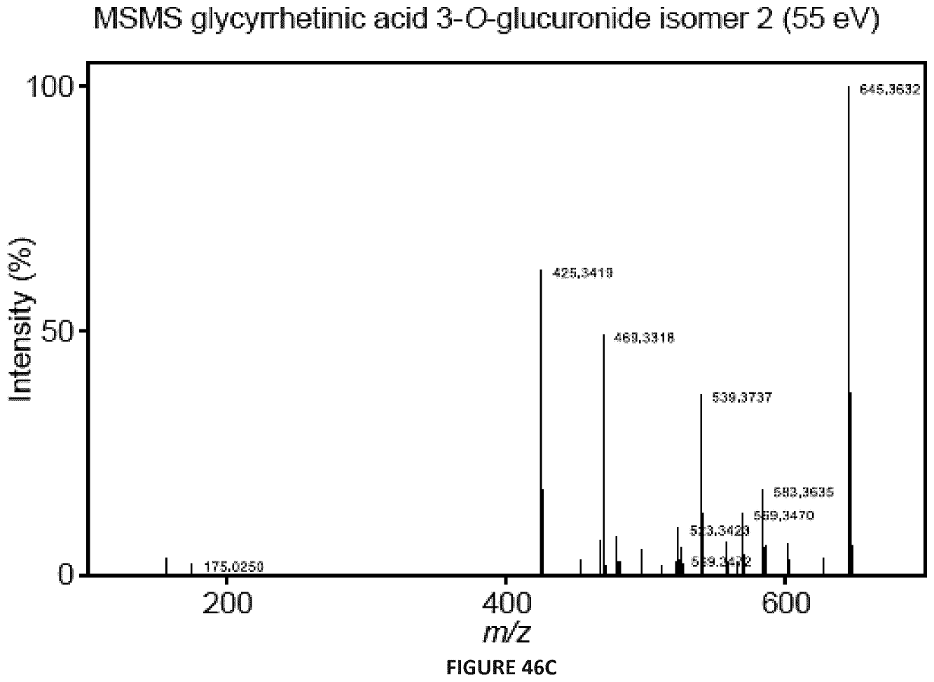

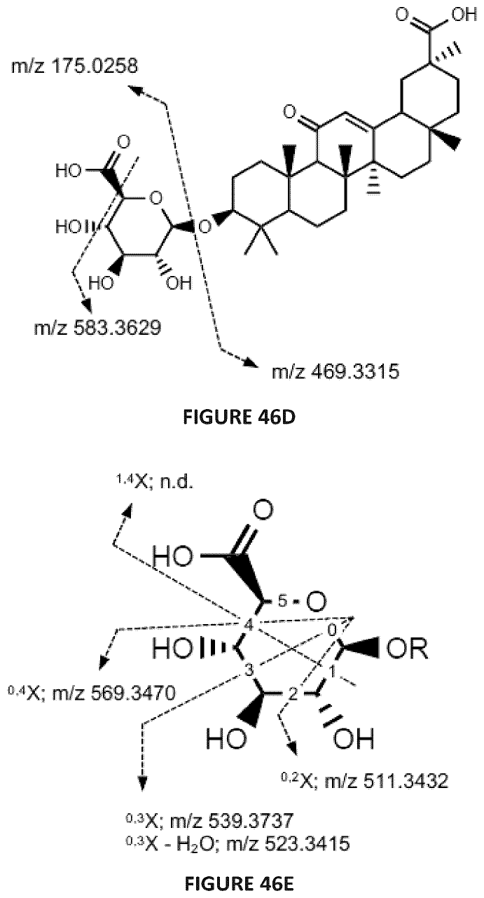

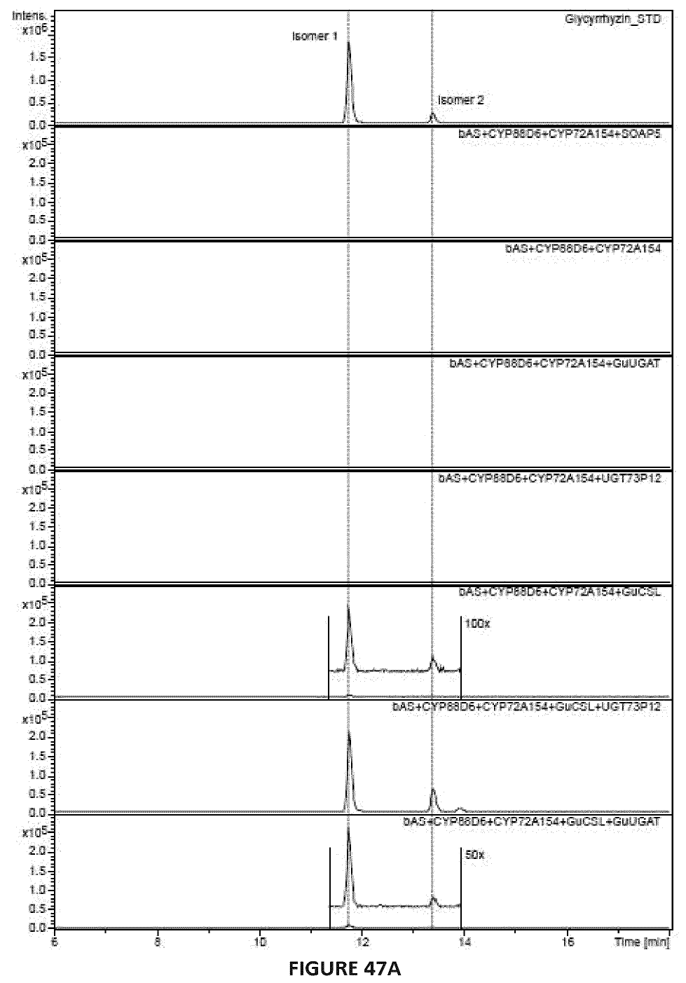

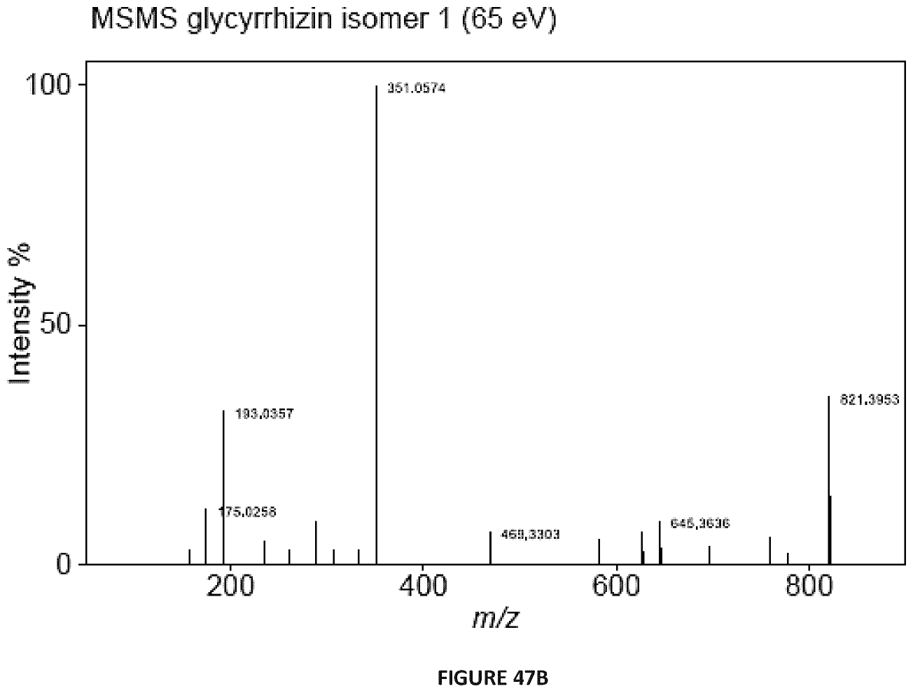

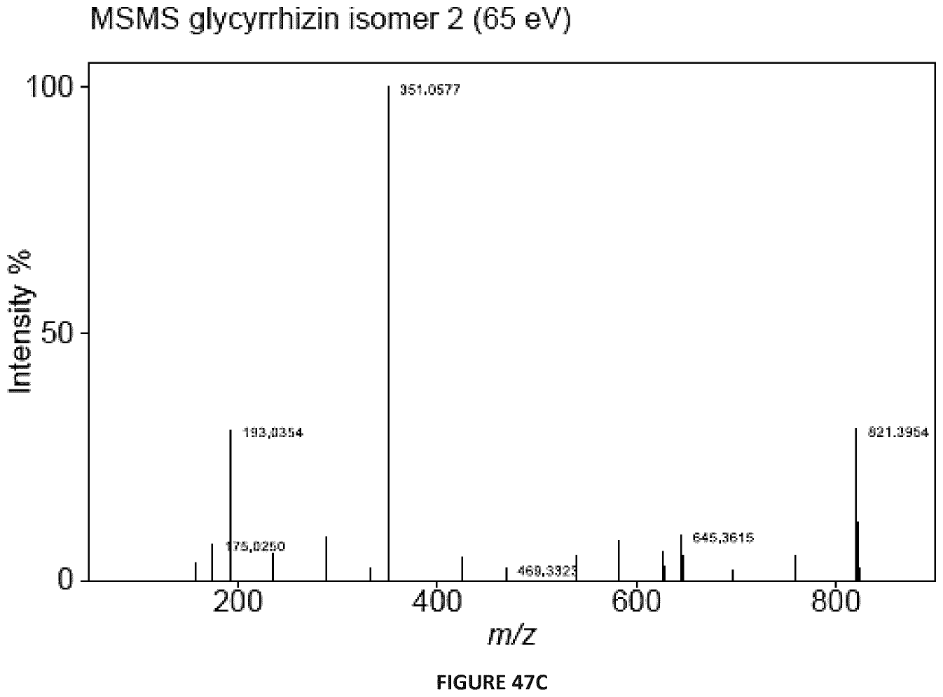

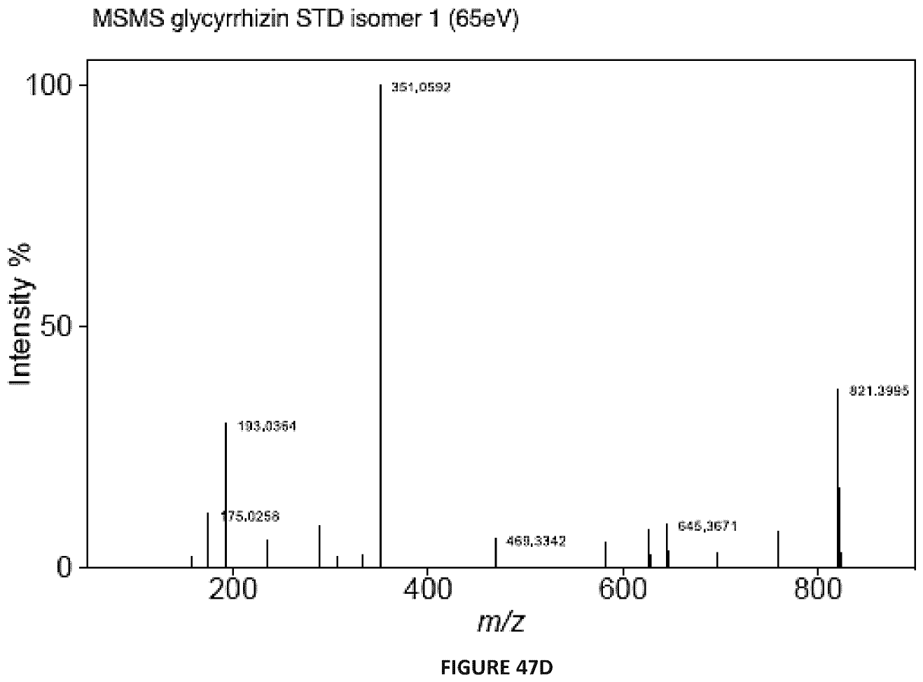

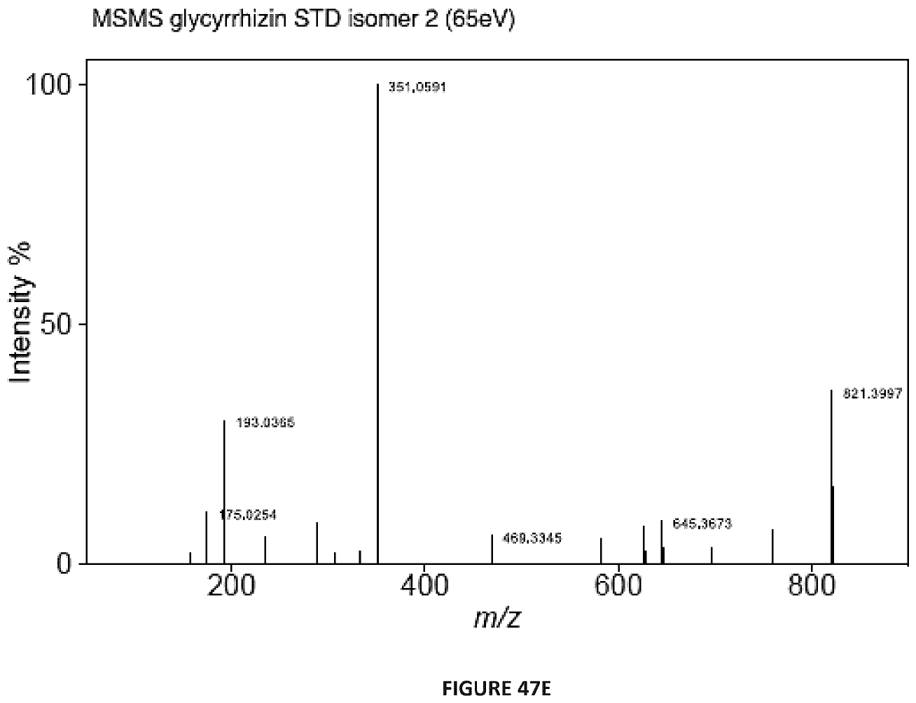

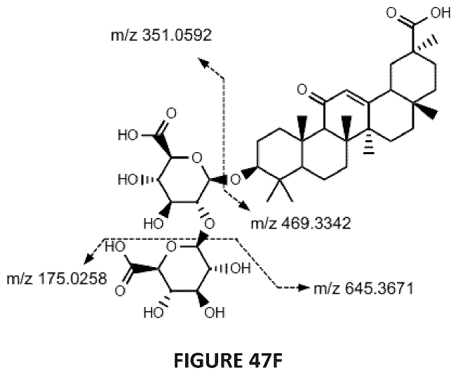

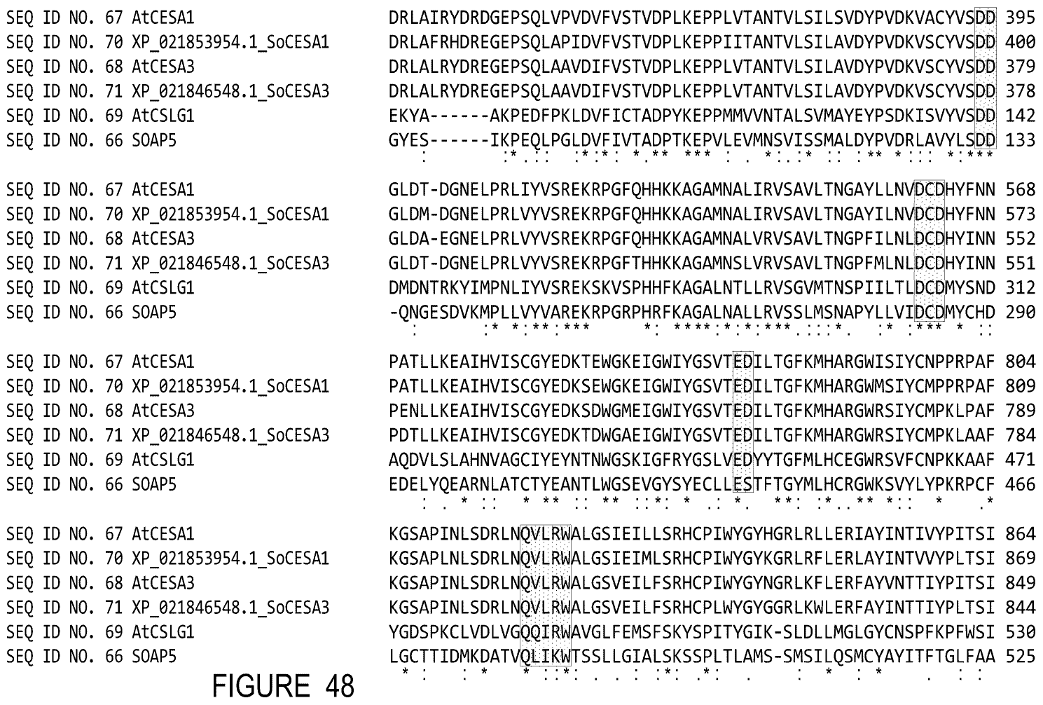

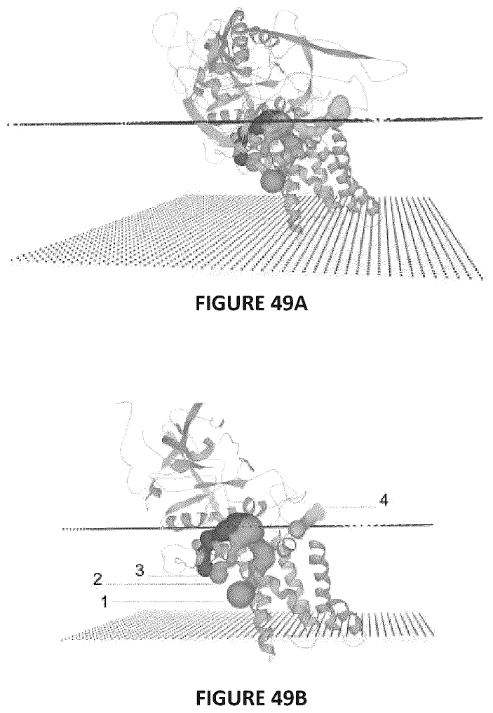

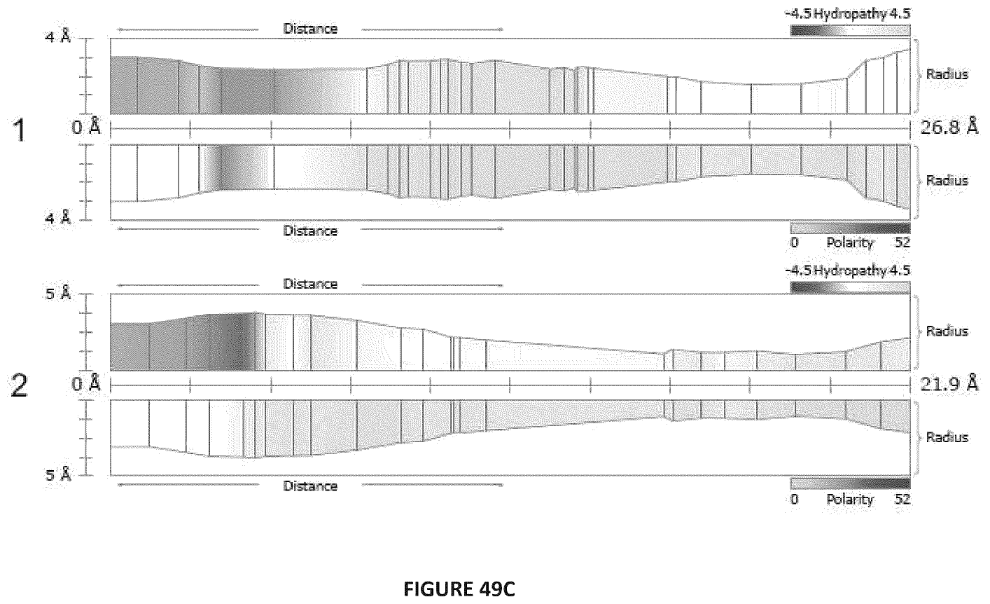

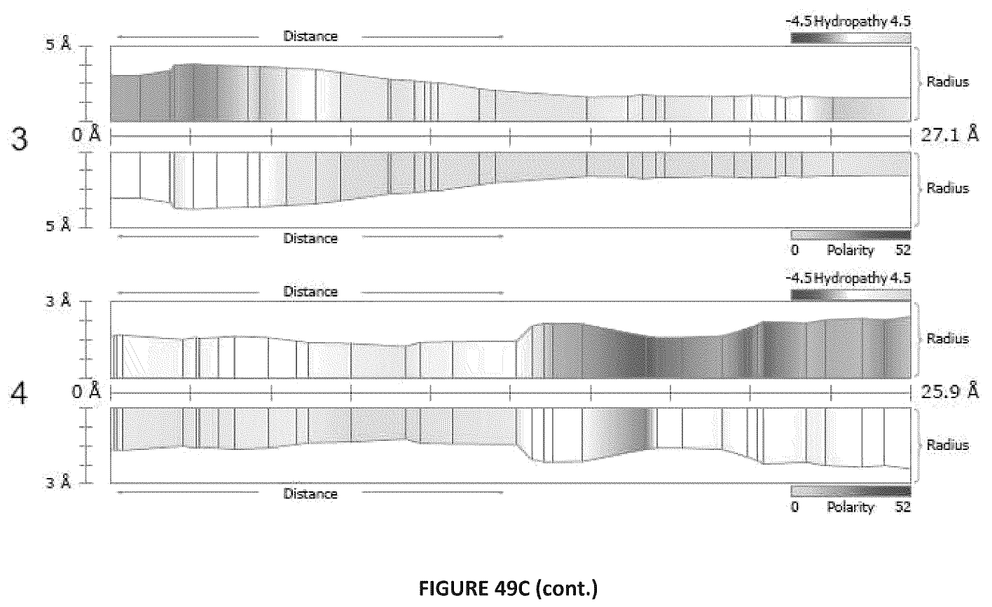

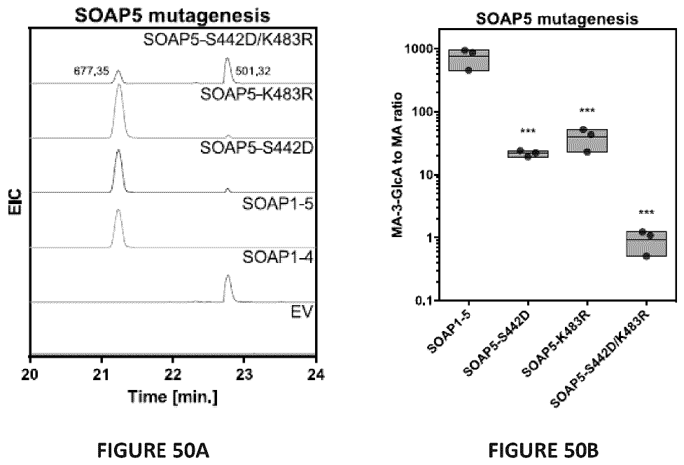

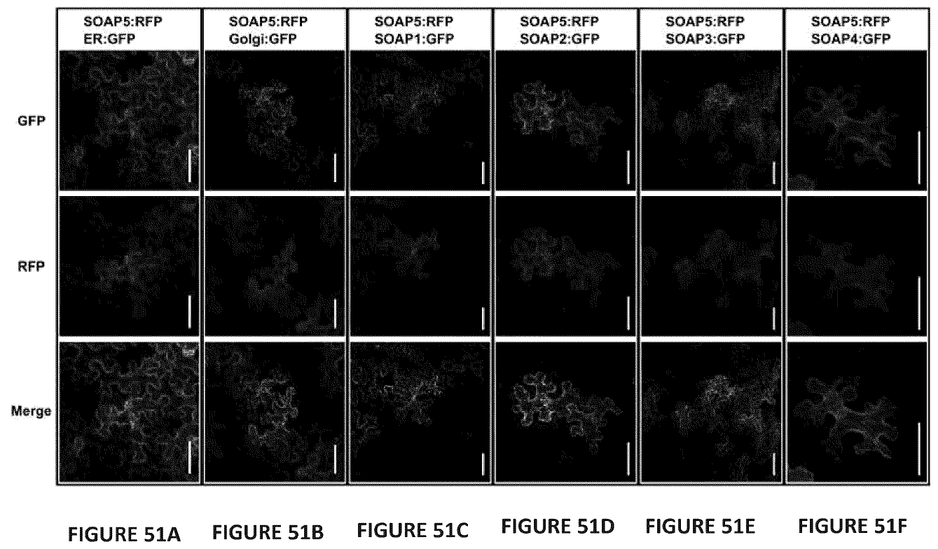

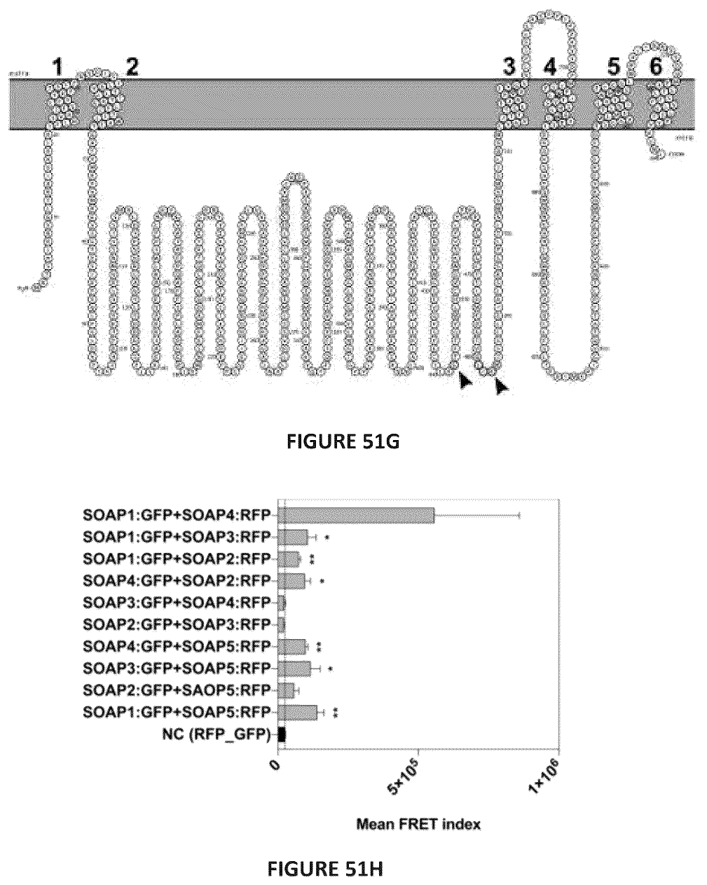

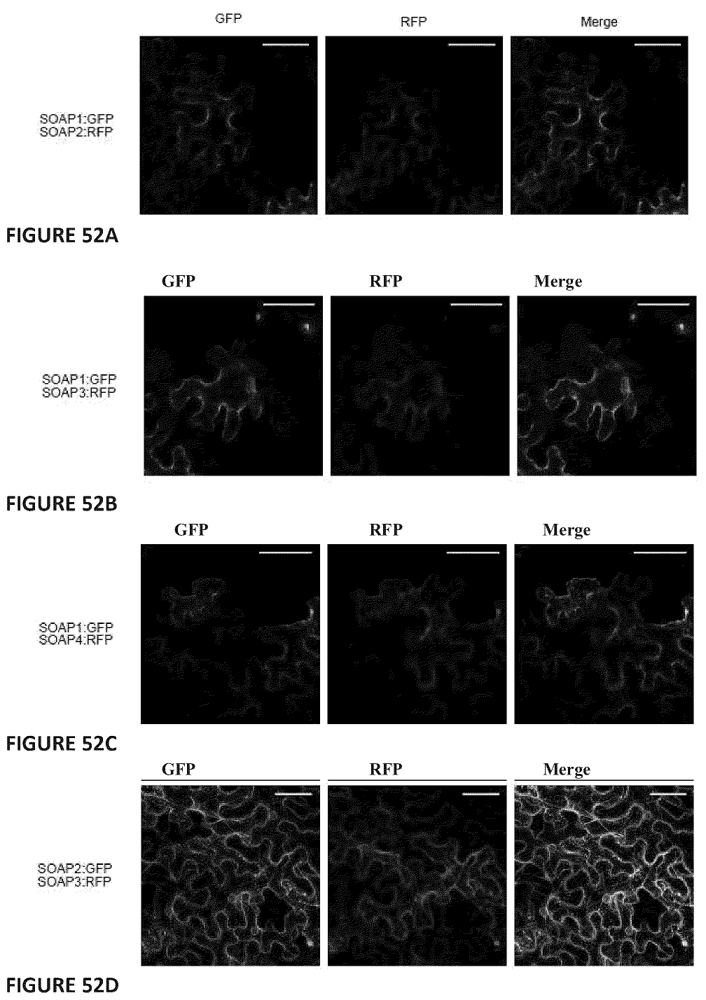

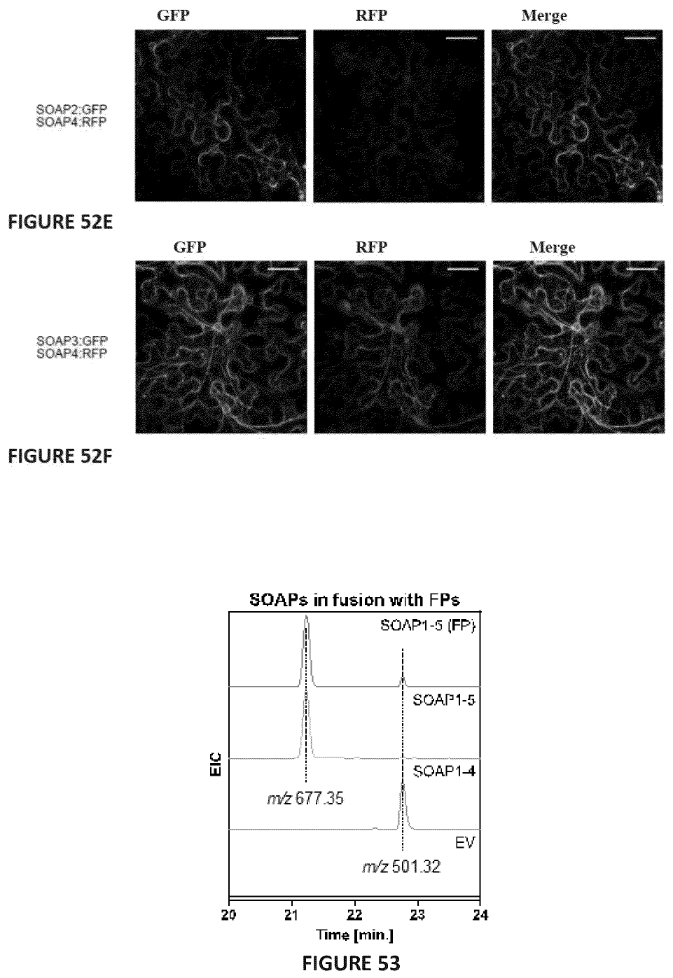

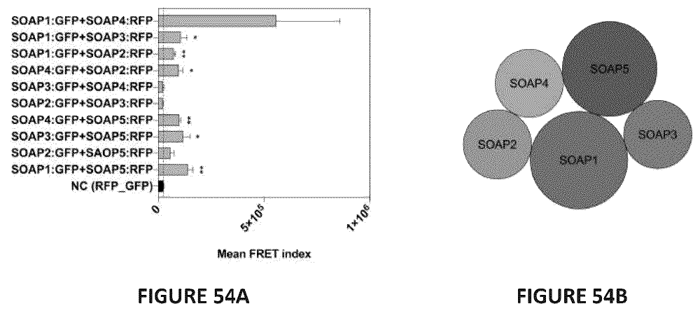

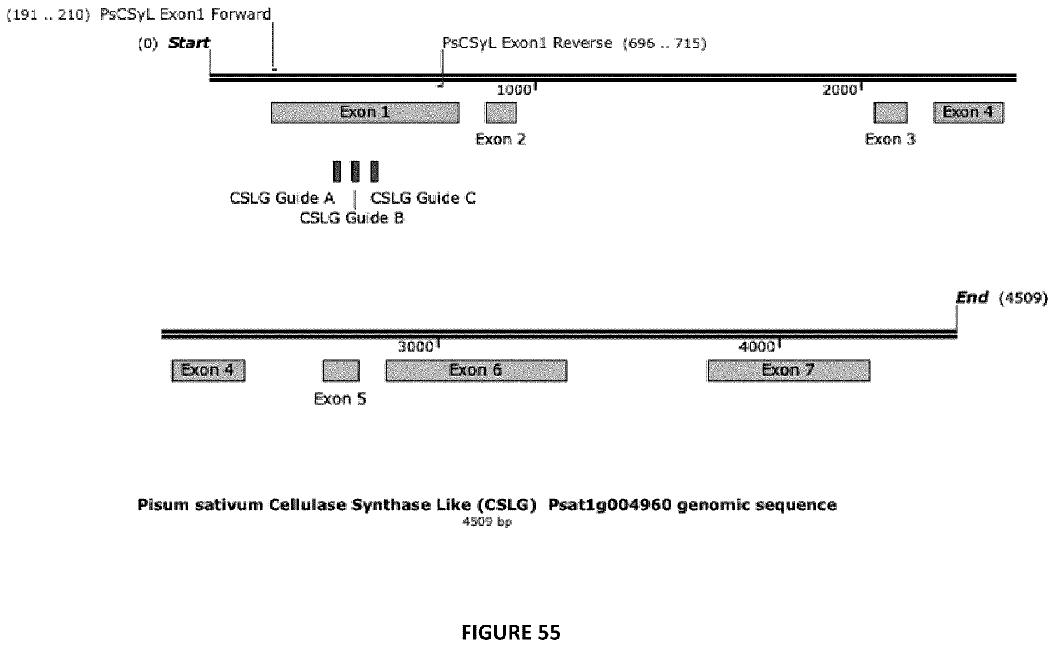

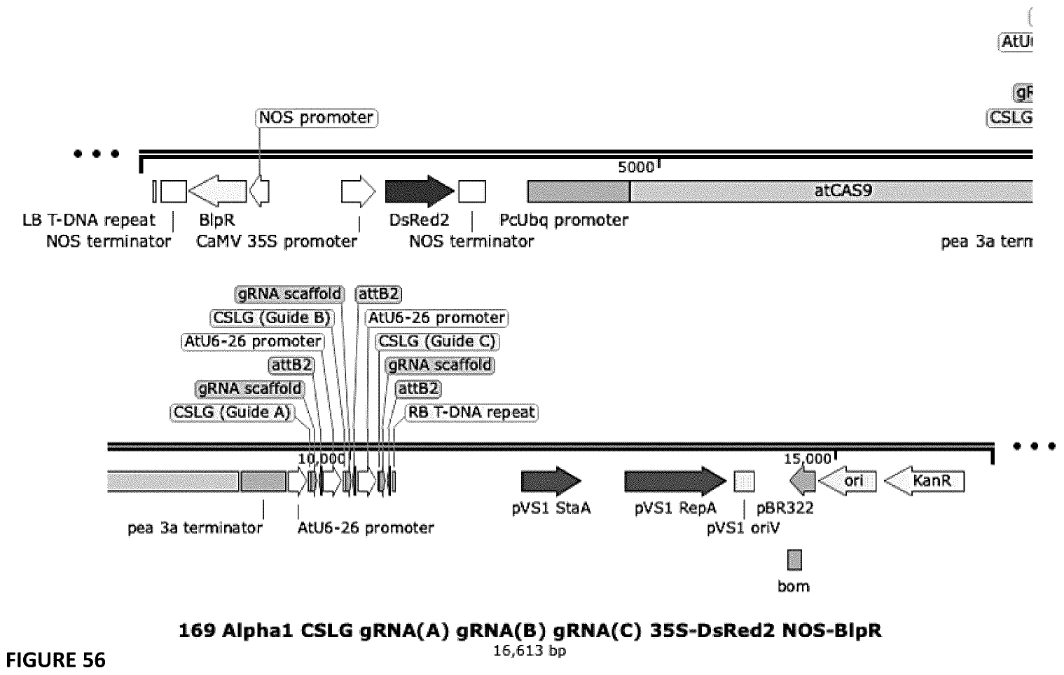

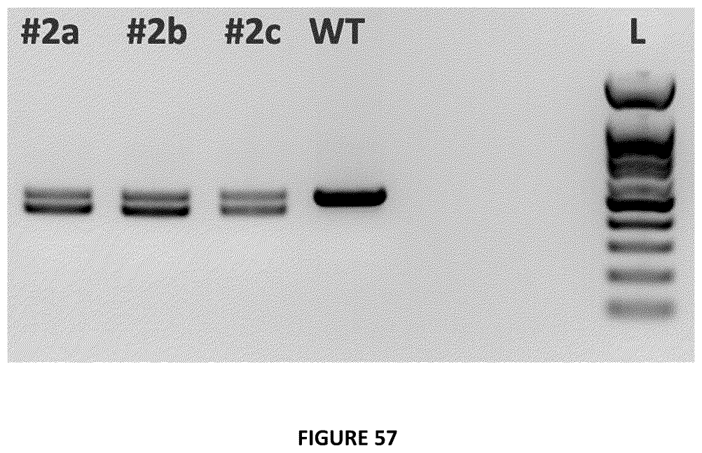

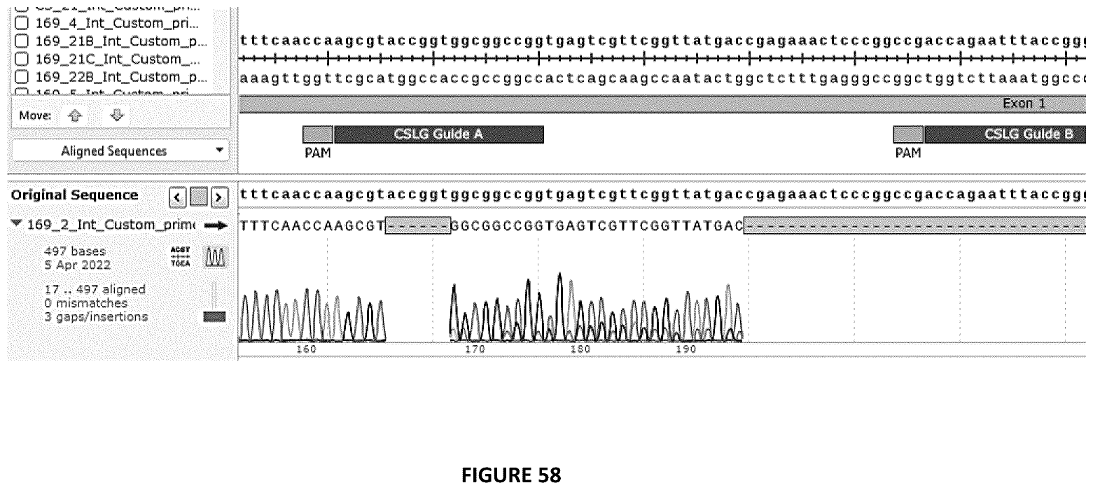

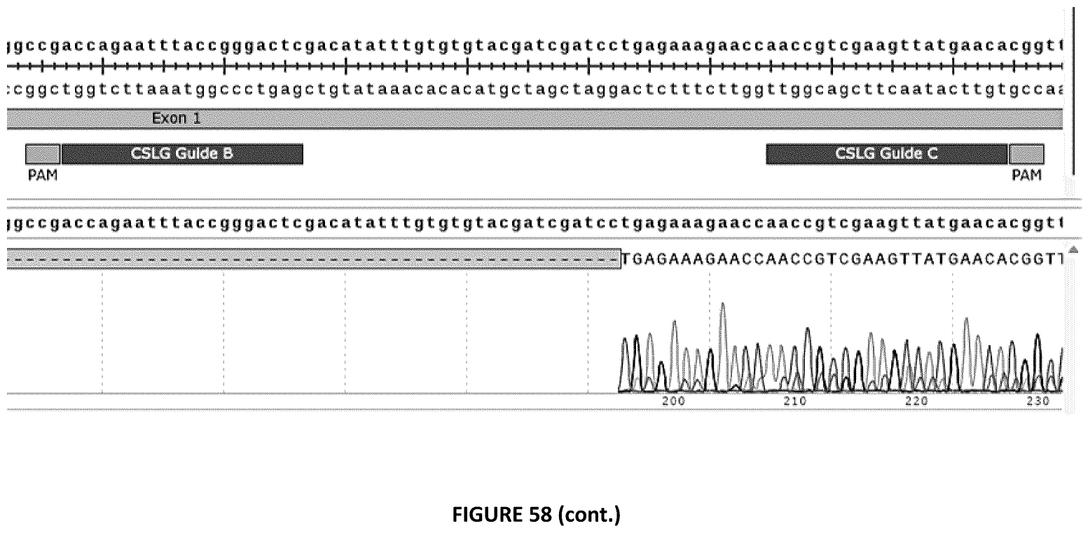

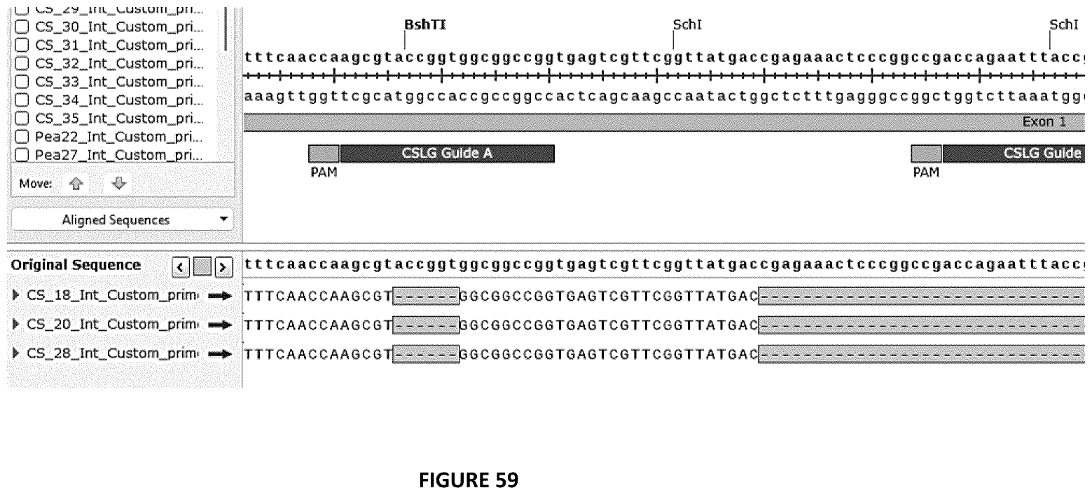

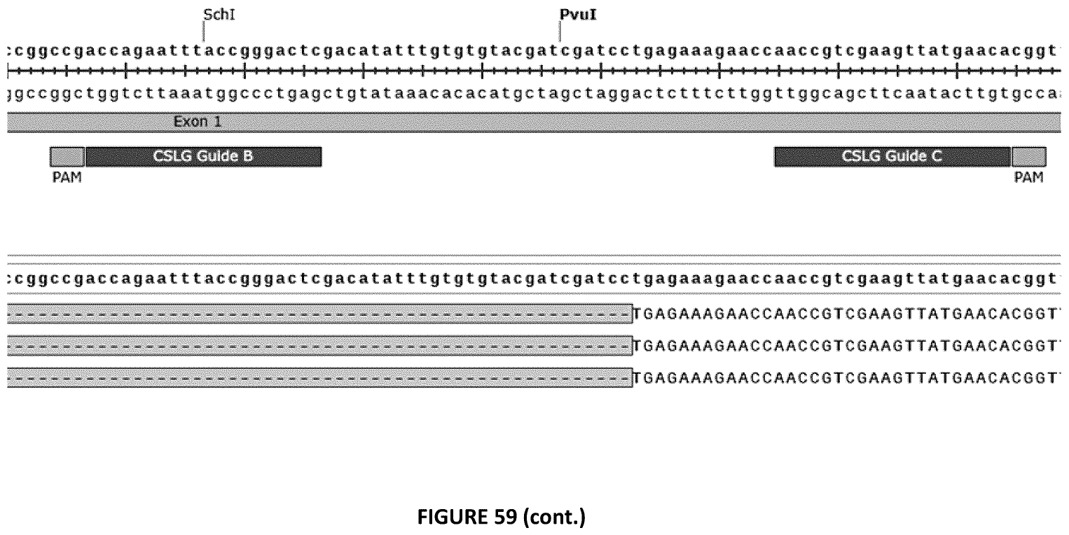

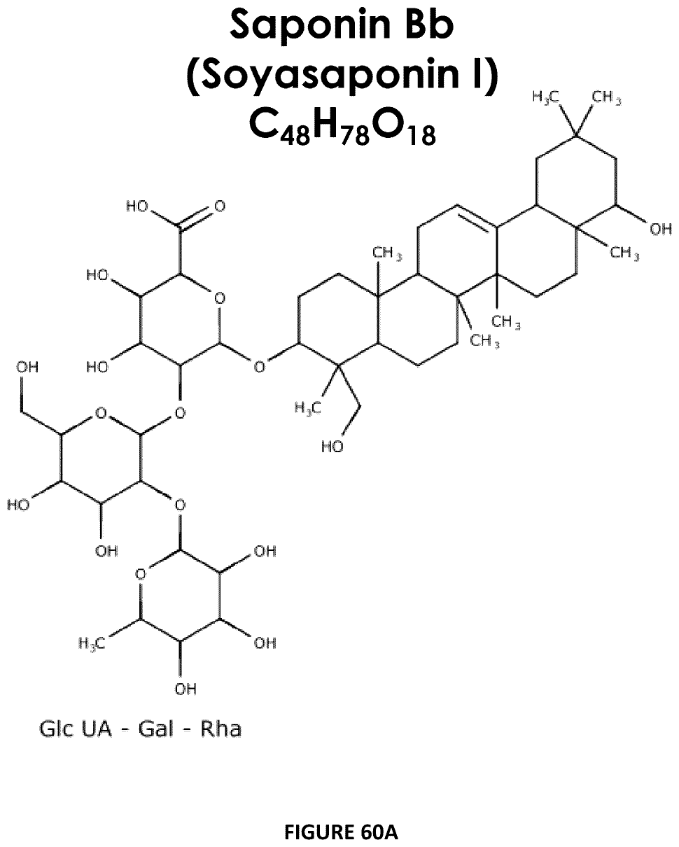

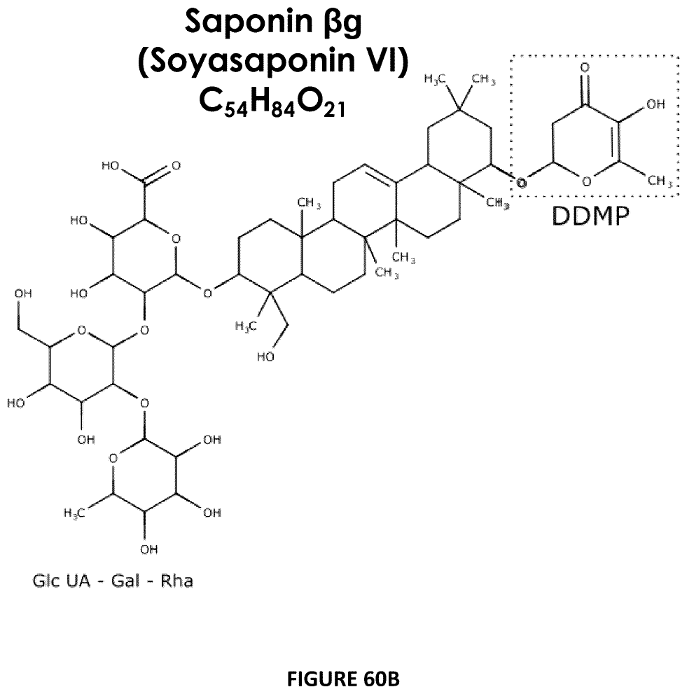

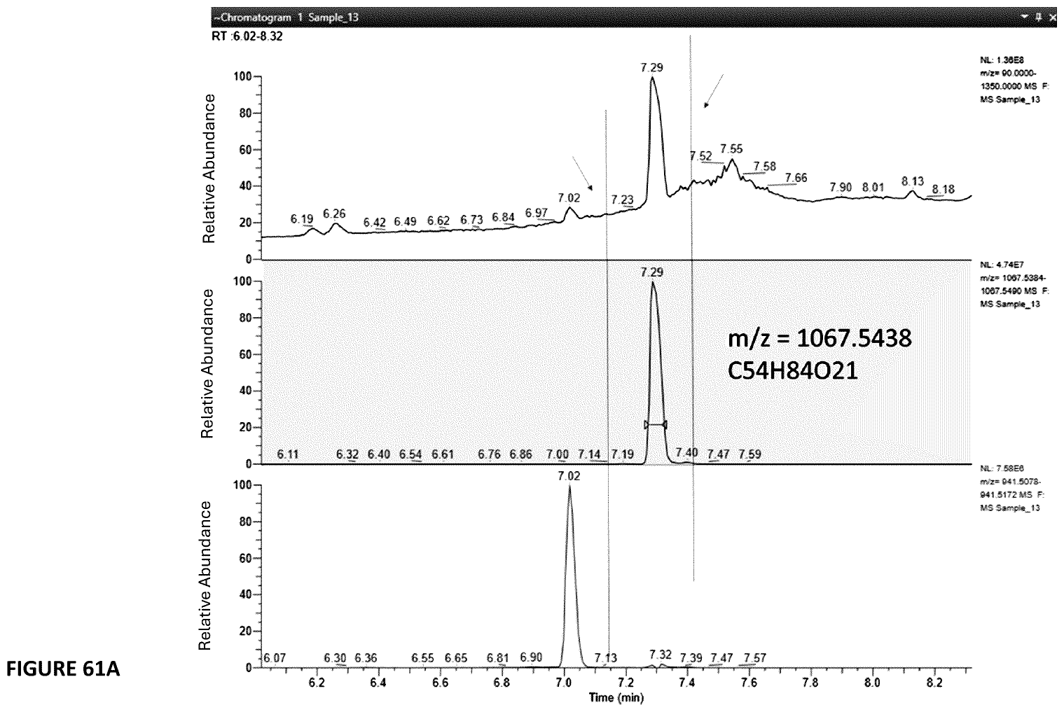

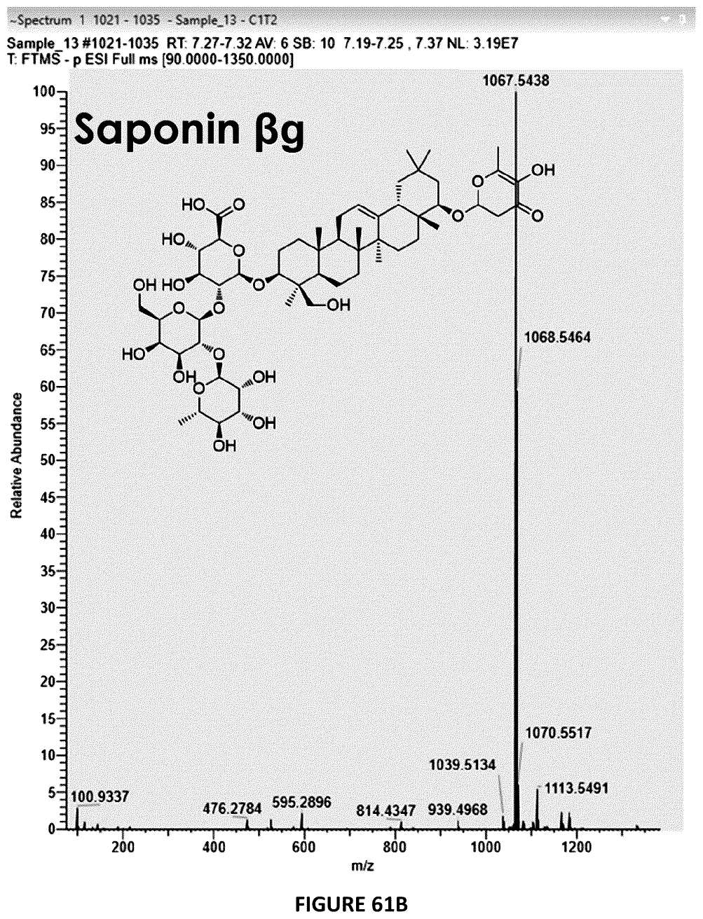

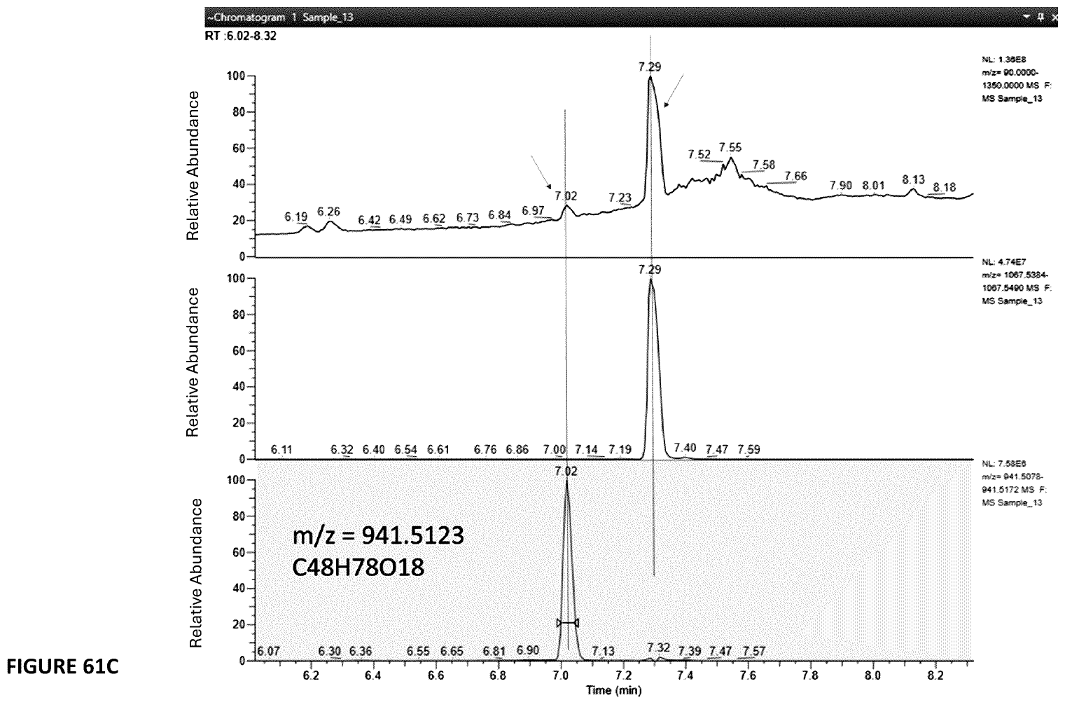

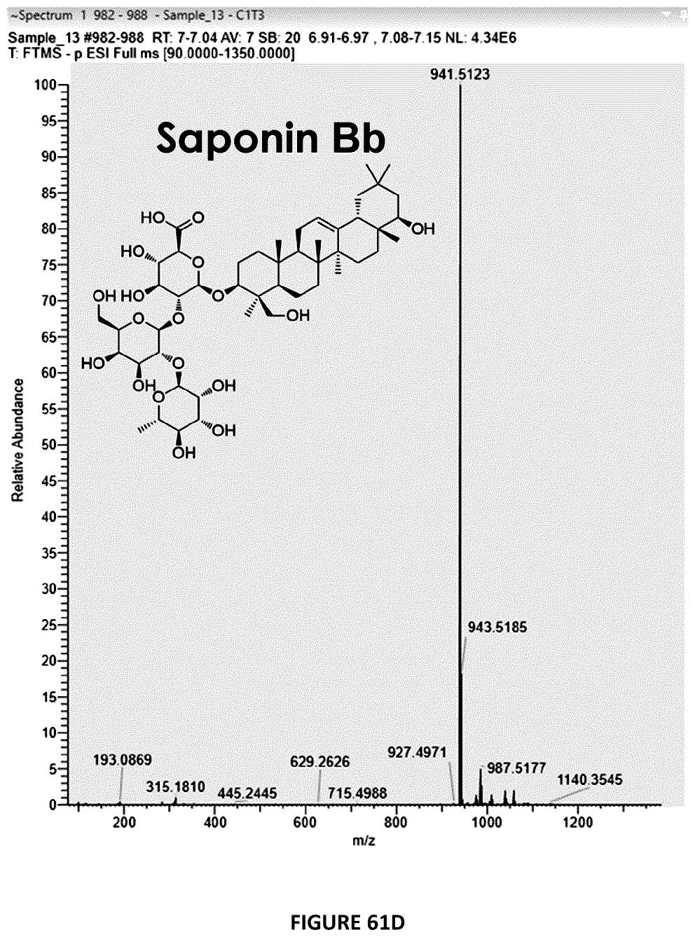

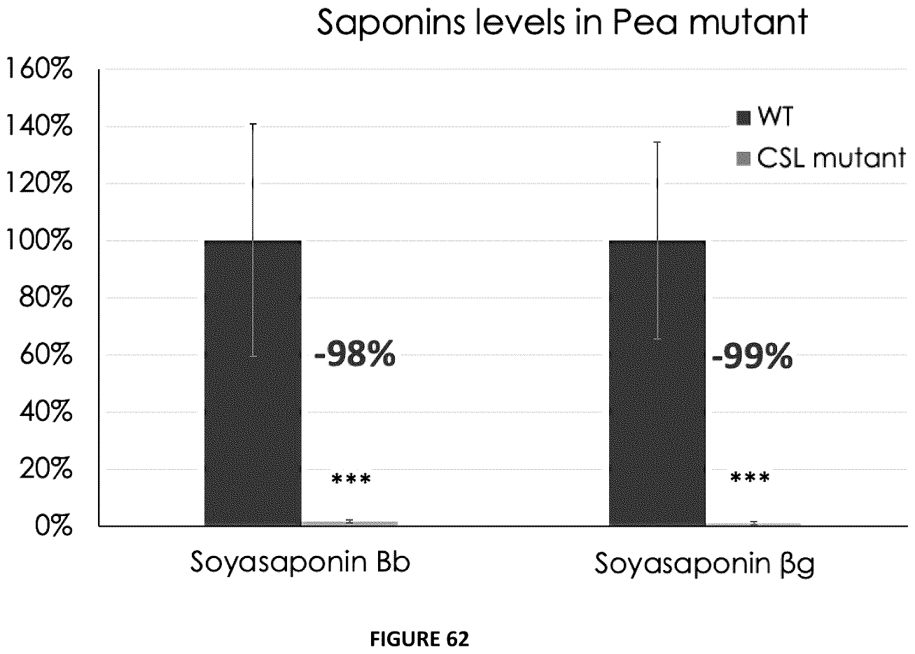

The structure and stereochemistry of the aglycone unit were determined by 1D ¹H and ¹³C, 2D homonuclear COSY and 2D heteronuclear HSQC and HMBC spectra. Identification of the monosaccharide units required recording also 2D homonuclear TOCSY spectra so as to complete the assignment and delineate connectivities between ¹H signals within the individual sugar units. Correlations observed in HMBC spectra between ¹H and ¹³C that are 2-3 bonds apart, are shown in FIG. 21 and FIG. 35D for Yossoside V and MA-3-GlcA, respectively.

¹H 1D NMR spectra were acquired using 16 k data points and a recycling delay of 2.5 s. ¹³C spectral-editing DEPTQ NMR spectra were acquired using 65 k data points and a recycling delay of 3 s.

2D ¹H-¹H COSY, TOCSY and ROESY spectra were acquired using 16384-8192 (t₂)×400-512 (t₁) data points. 2D TOCSY spectra were acquired using isotropic mixing times of 100-300 ms. A T-ROESY experiment was used in this study, TOCSY-less ROESY that effectively suppresses TOCSY transfer in ROESY experiments. 2D T-ROESY spectra were recorded using spin lock pulses of 100-400 ms.

2D ¹H-¹³C HSQC and HMBC spectra were recorded using 4096 (t₂)×400-512 (t₁) data points. Multiplicity editing HSQC enables differentiating between methyl and methine groups that give rise to positive correlation, vs methylene groups that appear as negative peaks. HMBC delay for evolution of long range couplings was set to observe long range couplings of J_{H,C}=8 Hz.

Chemical shift assignment was based on combined information derived from HMBC connectivities (FIG. 21 and FIG. 35D) and TOCSY correlations (data not shown).

¹H and ¹³C chemical shift values of Yossoside V and MA-3-GlcA are shown in Table 1. As expected, chemical shift values of the aglycone unit of the two compounds nicely fit and similar HMBC correlation pattern is observed (FIG. 21 and FIG. 35D). Assignment of the protons as axial or equatorial was based on the observed vicinal J couplings; large value (>10 Hz) indicates on axial protons, further supported by correlations observed in ROESY spectra.

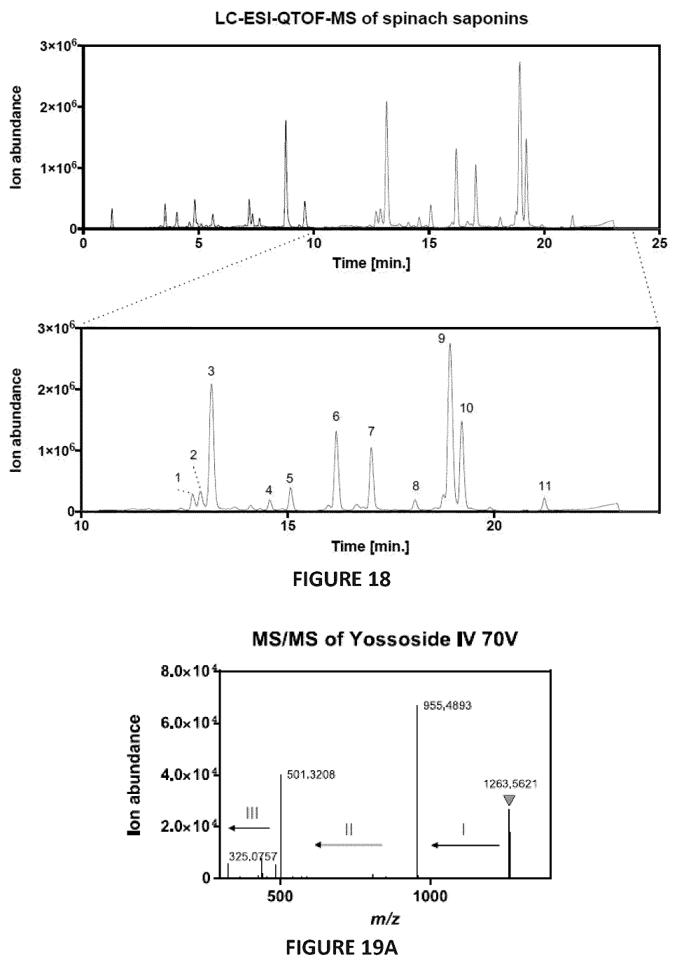

HMBC correlations in aglycone region are in accordance with those observed in other triterpenoid saponins (H. Schröder et al., A triterpene saponin from Herniaria glabra. Phytochemistry. 34, 1609-13 (1993); E. P. Mazzola et al., Utility of coupled-HSQC experiments in the intact structural elucidation of three complex saponins from Blighia sapida. Carbohydr Res. 346, 759-68 (2011)). The linkage site between medicagenic acid and glucuronic acid in MA-3-GlcA was supported by strong HMBC connectivity between the anomeric hydrogen of GlcA and C3 of the aglycone, as well as a complementary correlation observed between MA-H3/anomeric C1 of GlcA (red arrow in FIG. 18D). Similarly, linkage sites between the saccharide units were determined based on complementary 3-bond HMBC connectivities observed between the anomeric hydrogen/carbon of one sugar and the proton/carbon of the directly attached glycan as indicated by the interglycan arrows between Fuc-Rha-Glc (FIG. 21). The addition of xylose in Yossoside V (Xyl connected to GlcA) seems to restrict the conformational space of GlcA as is indicated by a lack of previously observed correlation between GlcA and the aglycone and lack of J couplings within GlcA. However, chemical shift prediction further supports the assignment of GlcA in Yossoside V.

### RNA-Seq Library Preparation and Data Analysis

RNA-Seq libraries were prepared as described previously (S. Zhong et al., High-throughput illumina strand-specific RNA sequencing library preparation. Cold Spring Harb Protoc. 8, 940-9 (2011)) with minor modifications. Briefly, 5 μg of total RNA was used for poly(A) RNA capture using Dynabeads Oligo (dT)₂₅ (Invitrogen), fragmented at 94° C. for 5 minutes and eluted. The first-strand cDNA was synthesized using reverse transcriptase SuperScript III (Invitrogen) with random primers and dNTP, whereas the second-strand cDNA was generated using DNA polymerase I (Enzymatics) using dUTP. After end-repair (Enzymatics), dA-tailing with Klenow 3′-5′ (Enzymatics) and adapter ligation (Quick T4 DNA Ligase, NEB), the dUTP-containing second-strand was digested by uracil DNA glycosylase (Enzymatics). The resulting first-strand adaptor-ligated cDNA was used for PCR enrichment (NEBNext High-Fidelity PCR Master Mix, NEB) for 14 cycles. Indexed libraries were pooled and sequenced (paired-end, 125 bp) on a single lane of HiSeq2500 (Illumina) at the Crown Institute for Genomics, The Nancy and Stephen Grand Israel National Center for Personalized Medicine, Weizmann Institute of Science. The quality of the raw data was assessed using FastQC (S. W. Wingett, S. Andrews, FastQ Screen: A tool for multi-genome mapping and quality control. Version 2. F1000Res. 7, 1338 (2018)). Raw reads were aligned against version 1.0.1 of the spinach reference genome (The Beta vulgaris resource (http://bvseq.boku.ac.at/Genome/Download/index.shtml)) using Tophat v2.0.13 (C. Trapnell, L. Pachter, S. L. Salzberg, TopHat: discovering splice junctions with RNA-Seq. Bioinformatics. 25, 1105-11 (2009)). Quantification was performed using HTSeq (S. Anders S, P. T. Pyl, W. Huber, HTSeq—a Python framework to work with high-throughput sequencing data. Bioinformatics. 31, 166-9 (2015)) followed by normalization and differential gene expression analysis using DESeq2 (M. I. Love, W. Huber, S. Anders, Moderated estimation of fold change and dispersion for RNA-seq data with DESeq2. Genome Biol. 15, 550 (2014)).

### Co-Expression Analysis

Co-expression analysis was done using CoExpNetViz software (O. Tzfadia, T. Diels, S. De Meyer, K. Vandepoele, A. Aharoni, Y. Van de Peer, CoExpNetViz: Comparative Co-Expression Networks Construction and Visualization Tool. Front Plant Sci. 6, 1194 (2016)). Briefly, spinach bAS (SOAP1), CYP716A268 (SOAP2) and CYP716A268v2 (SOAP2-like) were used as ‘baits’ in co-expression analysis with correlation threshold set at 5 and 95 for lower ad higher percentile rank, respectively. Lists of co-expressed genes was additionally filtered according to correlation coefficient (r >0.9; PCC) for each bait (Table 14). The analysis was performed using spinach RNA-Seq transcriptome data from different tissues and developmental stages (SO4WOLLD—mature leaf from four week old plant grown in long day; SO8WOLSL—mature leaf from eight week old plant grown in short day; SO4WYLLD—young leaf from four week old plant grown in long day; SO8WOLSL—young leaf from eight week old plant grown in short day; SO4WFBLD—flower bud from four week old plant grown in long day). The co-expression network was visualized with the Cytoscape software (P. Shannon P et al., Cytoscape: a software environment for integrated models of biomolecular interaction networks. Genome Res. 13, 2498-504 (2003)).

### Cloning

Phusion High-Fidelity DNA Polymerase (New England Biolabs) was used for all PCR amplification steps according to the manufacturer's instructions. Restriction enzymes and T4 Ligase used for cloning were purchased from New England Biolabs. Oligonucleotide primers were purchased from Sigma-Aldrich. DNA excised from agarose gels was purified using the Gel/PCR extraction kit (Hy-Labs). E. coli TOP10 cells (Invitrogen) were used for plasmid isolation prior to transformation into other heterologous hosts. Plasmid DNA was isolated from E. coli cultures using the AccuPrep® Plasmid Mini Extraction Kit (Bioneer). For a list of primers used for cloning, see Table 11.

### Construct Preparation for VIGS

For the silencing of candidate genes in spinach, all gene sequences (200-400 bp) were amplified from spinach leaf cDNA template. Purified amplicons were inserted into modified pTRV2 vector (˜200 bp of magnesium chelatase subunit H inserted with EcoRI) digested with SacI.

GoldenBraid System and Expression in N. benthamiana

For N. benthamiana transient expression of multiple genes from the same Agrobacterium strain, multigene constructs were created using GoldenBraid 3.0 (GB) system (M. Vazquez-Vilar et al., GB3.0: a platform for plant bio-design that connects functional DNA elements with associated biological data. Nucleic Acids Res. 45, 2196-2209 (2017)). In the first step, all the CDS and tomato Ubiquitin promoter and terminator were domesticated by removing BsaI and BsmBI restriction sites and inserting them into pUPD2 GoldenBraid entry vector. In the next step, genes were subcloned together with terminator and promoter into pDGB3al or pDGB3α2 GB vector. First level α vectors allow transient expression of a single gene in N. benthamiana. Subsequently, pDGB3α1:SOAP1 was combined with pDGB3α2:SOAP2 into pDGB3Ω1:SOAP1+SOAP2 and pDGB3α1:SOAP3 with pDGB3α2:SOAP4 into pDGB3Ω2:SOAP3+SOAP4. Combination of the omega vectors allows expression of four genes providing production of medicagenic acid in N. benthamiana. Other genes, cellulose synthase like G (SOAP5), glycosyltransferases (SOAP6-9) and acyltransferase (SOAP10) were used in alpha vectors. Genes from C. quinoa, B. vulgaris, L. japonicus, Glycine max (G. max,) M. sativa and G. uralensis were cloned using same approach. For cDNA template preparation RNA extracted from 2-week-old seedlings (L. japonicus, G. max, M sativa and G. uralensis), mature leaves (B. vulgaris) or from leaves surrounding flower buds (C. quinoa) was used.

Cloning into Yeast System (pESC)

For the expression of SOAP1 (bAS), CYPs (SOAP2-4) and CSLG (SOAP5) in S. cerevisiae, the sequences were amplified from cloned pDGB3a vectors carrying the sequence of interest and the purified amplicons were inserted into series of pESC (Amp^{R}) plasmids allowing simultaneous expression of two genes from one plasmid. SOAP1 and SOAP2 were inserted into pESC-HIS plasmid using NotI/SacI and BamHI/SalI restriction enzymes, respectively. SOAP3 and SOAP4 were inserted into pESC-LEU plasmid using NotI/SacI and BamHI/XhoI restriction enzymes, respectively. SOAP5 and SoUGD1 were inserted into pESC-LEU plasmid using NotI/SacI and SalII/XhoI restriction enzymes, respectively.

Cloning into pET-28b and Expression in E coli

SOAP10 was cloned into the pET-28b (Kan^{R}) vector using NdeI/NotI restriction sites and expressed in E. coli BL21 (DE3) cells.

In Vitro Enzymatic Assay with SOAP10

Bacterial cells transformed with pET-28b:SOAP10 were grown in LB medium at 37° C. When cultures reached A600=0.6, protein expression was induced with 200 μM of isopropyl-1-thio-β-D-galactopyranoside (IPTG) at 15° C., for 24 h. Bacterial cells were lysed by sonication in 50 mM Tris-HCl pH 8.0, 10% glycerol, 0.1% Triton X-100, 1 mM PMSF and 100 mg/mL lysozyme.

The fraction of spinach desacetyl saponins was used as a substrate in SOAP10 enzyme assay. In a 200 μL tube 6 μL of cell lysate was mixed with 2 μL of 6.18 mM acetyl coenzyme A (Acetyl-CoA) and 2 mL of saponin substrate (10 μg). Reaction was carried out for 2.5h at 28° C. Cell lysate without SOAP10 was used as a control. The reactions were stopped by addition of 20 μL methanol, followed by brief vortex. Finally, the extracts were centrifuged for 10 min. at 14,000×g and analyzed by LC-MS, as described above.

### VIGS Procedure

Virus Induced Gene Silencing was performed as described previously (M. Senthil-Kumar, K. S. Mysore, Tobacco rattle virus-based virus-induced gene silencing in Nicotiana benthamiana. Nat Protoc. 9, 1549-62 (2014)). Briefly, pTRV1 and pTRV2 constructs harboring sequence for silencing were transformed into Agrobacterium tumefaciens (GV3101) electrocompetent cells. Transformants were grown on LB plates containing 50 μg/mL kanamycin and 50 μg/mL gentamicin at 28° C. 10 mL of LB medium supplemented with antibiotics was inoculated with a single colony and grew O/N at 28° C. Cells were centrifuged at 3,000×g for 10 min. and supernatant removed. Pellet was resuspended in 5 mL of infiltration medium (100 mM MES buffer, 2 mM Na₃PO₄·12H₂O, 100 μM acetosyringone) and centrifuged again. Pellet was resuspended again in 10 mL of infiltration medium and incubated at room temperature for 2h. Agrobacterium suspensions (OD600=0.3 for each strain) were infiltrated into the underside of 1-week old spinach cotyledons (one per plant) with a needleless 1 mL syringe. Plants were grown in 16h photoperiod at 23° C. After 2 weeks first signs of silencing of Magnesium Chelatase subunit H (CHCL) were visible. Plants were grown for another 2 weeks prior tissue collection. Leaves were harvested, frozen in liquid nitrogen and stored at −80° C. for later processing. Biological replicates consisted of 4 leaves all from different plants. Table 10 below provides the spinach and red beet nucleic acid sequences used to silence CSLGs in red beet and spinach

TABLE 10
VIGs Sequences

| | SEQ | |
|---|---|---|
| | ID | |
| Name | NO: | Nucleic Acid Sequence |
| SoCSLG | 106 | TTCCGTAGCCATCTTCTCGCTCTTCTACTACCGTTTCACTTCCTTCTTCA |
| (SOAP5) | | ACTCCGACATCTCCATACTTGCTTACTCCTTACTCACCACCGCCGAACT |
| VIGS | | CTTCTTAACCTTTCTATGGGCTTTTACTCAGGCTTTCCGGTGGCGTCCCG |
| spinach | | TAATAGGGAAGTCTCCGGGTACGAATCCATCAAACCCGAACAACTAC |
| | | CGGGTTTGGATGTCTTCATTGTCACTGCTGACCCGACAAAGGAGCCAGT |
| | | TCTGGAGGTGATGAACTCCGTGATATCATCCATGGCGTTGGATTATCCG |
| | | GTTGATAGACTGGCGGTTTACTTGTCGGATGACGGTGGTTCTCCGTTGT |
| | | CGAAGG |
| BvCSLG | 107 | CCTTCGGCTCCTCCACAAAATTCATTGCGTCAGTTAGTTCAAACTCCAA |
| VIGS | | GCAAAATATGGCCTTGAAGGAAATGACAAGAGACGACTTGTTAGAAGA |
| red beet | | GGCTAAAAATTTGGCTACTTGTGCATATGAATCAAACACTGAATGGGG |
| | | TAACAAGATTGGATATTCGTATGAGTGTTTGTTGGAGAGTACATTTACC |
| | | GGATATCTCTTACATTGCAAAGGATGGATTTCCGTGTATCTTTACCCAA |
| | | AAAGACCCTGCTTCTTAGGATGCACGACGATTGACATGAAAGATGCCA |
| | | TGGTTCAACTAATGAAATGGACCTCTGGATTACTAGGAGTTGGCATATC |
| | | AAAGTTTAGCCCTCTAACTTATGCCTTTTCGAGGATGTCTATATTACAA |
| | | AGCATGTGCTACGGTTACTTCACATTTTCAGCCCTTTTCGGAGTTTCGT |

### Site-Directed Mutagenesis of SOAP5

Site-directed mutagenesis of SOAP5 was performed using QuikChange Site-Directed Mutagenesis Kit (Agilent) according to the manufacturer's instructions.

Gene Expression Analysis qPCR

Gene expression analysis was performed with three/four biological replicates (n=3/4) for each VIGS silenced line. RNA isolation was performed by the Trizol method (Sigma-Aldrich). DNase I (Sigma-Aldrich)-treated RNA was reverse transcribed using a high-capacity cDNA reverse transcription kit (Applied Biosystems). Gene-specific oligonucleotides were designed with Primer Blast software (NCBI). The Translation Elongation Factor Alpha 1 (EF1-α) gene was used as an endogenous control. Oligonucleotides used are listed in Table 11.

TABLE 11
List of primers used

| | A | B | C | D | E | F | G |
|---|---|---|---|---|---|---|---|
| 1 | gene name | annotation | species | locus | purpose | primer name | sequence |
| 2 | OXIDOSQUALENE CYCLASE | | | | | | |
| 3 | SOAP1 | β-amyrin | Spinacia | Sp_107620_kqnh | VIGS | Sp107620_vF | CTAGAGCTCATGTTCAAGAAGTTAT |
| | | synthase, bAS | oleracea | | | | ACCC |
| 4 | | | | | | Sp107620_vR | TTCGAGCTCCCTCCTTTTGTGTAGT |
| | | | | | | | AA |
| 5 | | | | | expression in N. | Sp107620_GB_1F | GCGCCGTCTCGCTCGAATGTGGAG |
| | | | | | benthamiana | | GTTGAAGGTTGG |
| 6 | | | | | | Sp107620_GB_1R | GCGCCGTCTCGCTGTCTCATATGTG |
| | | | | | | | ATCTCCT |
| 7 | | | | | | Sp107620_GB_2F | GCGCCGTCTCGACAGCCTCGACGA |
| | | | | | | | CATTAAAG |
| 8 | | | | | | Sp107620_GB_2R | GCGCCGTCTCGCTCAAAGCTCAAGT |
| | | | | | | | AGAGTTGATAGAAGGTAA |
| 9 | | | | | expression in yeast | Sp107620_NotI_F | AAAAGCGGCCGCATGTGGAGGTTG |
| | | | | | | | AAGGTTGGA |
| 10 | | | | | | Sp107620_SacI_R | AAAAGAGCTCTCAAGTAGAGTTGAT |
| | | | | | | | AGAAGGTAATG |
| 11 | | | | | colocalization | Sp107620_GB_1F | GCGCCGTCTCGCTCGAATGTGGAG |
| | | | | | | | GTTGAAGGTTGG |
| 12 | | | | | | Sp107620_GB_CT | GCGCCGTCTCGCTCACGAACCAGT |
| | | | | | | R | AGAGTTGATAGAAGGTAATGA |
| 13 | CYTOCHROMES P450 | | | | | | |
| 14 | SoCYP1 | CYP715A56 | Spinacia | Sp_046690_sjiw | VIGS | Sp046690_vF | CTAGAGCTCCTTCGTCGTTGTTGTC |
| | | | oleracea | | | | GTCG |
| 15 | | | | | | Sp046690_vR | TTCGAGCTCCCTATGACGAGCCCAA |
| | | | | | | | GCAT |
| 16 | SoCYP2 | CYP72A656 | Spinacia | Sp_148230_dgra | VIGS | Sp148230_vF | CTAGAGCTCAGCTCTACTTTGCGTG |
| | | | oleracea | | | | CTCA |
| 17 | | | | | | Sp148230_vR | TTCGAGCTCTCTGGCCCTCATAGTT |
| | | | | | | | CGGA |
| 18 | SoCYP3 | CYP72A685 | Spinacia | Sp_085350_pxjs | VIGS | Sp085350_vF | CTAGAGCTCTTGTTGGGTTGCTTGT |
| | | | oleracea | | | | CCCT |
| 19 | | | | | | Sp085350_vR | TTCGAGCTCGGTCTTTGTTGGCACG |
| | | | | | | | AACC |
| 20 | SOAP2 | CYP716A268 | Spinacia | Sp_107660_kiqg | VIGS | Sp107660_vF | CTAGAGCTCTGGTGGGCGAGTCATT |
| | | | oleracea | | | | TGAA |
| 21 | | | | | | Sp107660_vR | TTCGAGCTCCAAGCCAGCCAGAAG |
| | | | | | | | GTGTA |
| 22 | | | | | expression in N. | Sp107660_GB_1F | GCGCCGTCTCGCTCGAATGGAACT |
| | | | | | benthamiana | | CTTCTTTATGTGTGG |
| 23 | | | | | | Sp107660_GB_1R | GCGCCGTCTCGCTCAAAGCTTAAGC |
| | | | | | | | AGCTACAGTTCGAGG |
| 24 | | | | | expression in yeast | Sp107660_BamHI | AAAAGGATCCATGGAACTCTTCTTTA |
| | | | | | | F | TGTGTGGG |
| 25 | | | | | | Sp107660_SalI_R | AAAAGTCGACTTAAGCAGCTACAGT |
| | | | | | | | TCGAGGATG |
| 26 | | | | | colocalization | Sp107660_GB_1F | GCGCCGTCTCGCTCGAATGGAACT |
| | | | | | | | CTTCTTTATGTGTGG |
| 27 | | | | | | Sp107660_GB_CT | GCGCCGTCTCGCTCACGAACCAGC |
| | | | | | | R | AGCTACAGTTCGAGGAT |
| 28 | SOAP3 | CYP72A655 | Spinacia | Sp_085340_meek | VIGS | Sp085340_vF | CTAGAGCTCGTTGCATTGCTTGTCC |
| | | | oleracea | | | | CTGG |
| 29 | | | | | | Sp085340_vR | TTCGAGCTCTGATTGCAGAAGGCGT |
| | | | | | | | AGGG |
| 30 | | | | | expression in N. | Sp085340_GB_1F | GCGCCGTCTCGCTCGAATGATAGAA |
| | | | | | benthamiana | | ATCGGGTATATTGTAAA |
| 31 | | | | | | Sp085340_GB_1R | GCGCCGTCTCGCTCAAAGCTTAGTC |
| | | | | | | | CCTGAGCTTATGTATAAT |
| 32 | | | | | expression in yeast | Sp085340_NotI_F | AAAAGCGGCCGCATGATAGAAATCG |
| | | | | | | | GGTATATTGTAAAATG |
| 33 | | | | | | Sp085340_SacI_R | AAAAGAGCTCTTAGTCCCTGAGCTT |
| | | | | | | | ATGTATAATGAG |
| 34 | | | | | colocalization | Sp085340_GB_1F | GCGCCGTCTCGCTCGAATGATAGAA |
| | | | | | | | ATCGGGTATATTGTAAA |
| 35 | | | | | | Sp085340_GB_CT | GCGCCGTCTCGCTCACGAACCGTC |
| | | | | | | R | CCTGAGCTTATGTATAATG |
| 36 | SOAP4 | CYP72A654 | Spinacia | Sp_040350_wdny | VIGS | Sp040350_vF | CTAGAGCTCTTTCAAAGAGCGCGAG |
| | | | oleracea | | | | TGGT |
| 37 | | | | | | Sp040350_vR | TTCGAGCTCGCCTGCACCAGAATTG |
| | | | | | | | TTGA |
| 38 | | | | | expression in N. | Sp040350_GB_1F | GCGCCGTCTCGCTCGAATGATTTCA |
| | | | | | benthamiana | | AAGAGCGCGAGT |
| 39 | | | | | | Sp040350_GB_1R | GCGCCGTCTCGTTGTCTCTTGACCA |
| | | | | | | | GCCAAG |
| 40 | | | | | | Sp040350_GB_2F | GCGCCGTCTCGACAACCTCAGTGG |
| | | | | | | | CCCTAAC |
| 41 | | | | | | Sp040350_GB_2R | GCGCCGTCTCGCTCAAAGCTTAAAA |
| | | | | | | | TCGATGTAAAATAATGTGGG |
| 42 | | | | | expression in yeast | Sp040350_BamHI | AAAAGGATCCATGATTTCAAAGAGC |
| | | | | | | F | GCGAGT |
| 43 | | | | | | Sp040350_XhoI_R | AAAACTCGAGTTAAAATCGATGTAAA |
| | | | | | | | ATAATGTGGG |
| 44 | | | | | colocalization | Sp040350_GB_1F | GCGCCGTCTCGCTCGAATGATTTCA |
| | | | | | | | AAGAGCGCGAGT |
| 45 | | | | | | Sp040350_GB_CT | GCGCCGTCTCGCTCACGAACCAAAT |
| | | | | | | R | CGATGTAAAATAATGTGGGC |
| 46 | GuCYP88D6 | CYP88D6 | Glycyrrhiza | Glyur000561s00023451.1 | expression in N. | GuCYP88D6_GB_1 | GCGCCGTCTCGCTCGAATGGAAGT |
| | | | uralensis | | benthamiana | F | ACATTGGGTTTGC |
| 47 | | | | | | GuCYP88D6_GB_1 | GCGCCGTCTCGCTCAAAGCCTAAG |
| | | | | | | R | CACATGATACCTTTATCAC |
| 48 | GuCYP72A15 | CYP72A154 | Glycyrrhiza | Glyur000890s00019071.1 | expression in N. | GuCYP72A154_GB | GCGCCGTCTCGCTCGAATGGATGC |
| | 4 | | uralensis | | benthamiana | 1F | ATCTTCCACACC |
| 49 | | | | | | GuCYP72A154_GB | GCGCCGTCTCGAAAGACCAATGGAT |
| | | | | | | 1R | TTTGATTGTG |
| 50 | | | | | | GuCYP72A154_GB | GCGCCGTCTCGCTTTCCAAAGATGA |
| | | | | | | 2F | TGCTGCA |
| 51 | | | | | | GuCYP72A154_GB | GCGCCGTCTCGTTGTCTCTTGCCCT |
| | | | | | | 2R | GCCAGG |
| 52 | | | | | | GuCYP72A154_GB | GCGCCGTCTCGACAACCGCAGCTTT |
| | | | | | | 3F | GCTGGC |
| 53 | | | | | | GuCYP72A154_GB | GCGCCGTCTCGCTCAAAGCTTACAG |
| | | | | | | 3R | TTTATGCAGAATGATGGG |
| 54 | GLYCOSYLTRANSFERASES | | | | | | |
| 55 | SOAP5 | Cellulose | Spinacia | Sp_076690_ejcm/Spo | VIGS | Sp076690_vF | CTAGAGCTCTTCCGTAGCCATCTTC |
| | | Synthase Like | oleracea | 12715 | | | TCGC |
| | | G2, CSLG2 | | | | | |
| 56 | | | | | | Sp076690_vR | TTCGAGCTCCCTTCGACAACGGAGA |
| | | | | | | | ACCA |
| 57 | | | | | expression in N. | Sp076690_GB_1F | GCGCCGTCTCGCTCGAATGGCAAC |
| | | | | | benthamiana | | TTCTCACATTCGC |
| 58 | | | | | | Sp076690_GB_1R | GCGCCGTCTCGGAAGACGGTTAAC |
| | | | | | | | AATGGCTC |
| 59 | | | | | | Sp076690_GB_2F | GCGCCGTCTCGCTTCACATCTTCCT |
| | | | | | | | CCATTCC |
| 60 | | | | | | Sp076690_GB_2R | GCGCCGTCTCGGCCTCTTTTCCCTG |
| | | | | | | | GCTACA |
| 61 | | | | | | Sp076690_GB_3F | GCGCCGTCTCGAGGCCTGGTCGTC |
| | | | | | | | CTCATCG |
| 62 | | | | | | Sp076690_GB_3R | GCGCCGTCTCGCTCAAAGCTTATAA |
| | | | | | | | CCATCCCTTAACAACAGG |
| 63 | | | | | expression in yeast | Spo12715F_NotI_ | AAAAGCGGCCGCATGGCAACTTCTC |
| | | | | | | F | ACATTCGCAA |
| 64 | | | | | | Spo12715R_SacI_ | AAAAGAGCTCTTATAACCATCCCTTA |
| | | | | | | R | ACAACAGGGTAG |
| 65 | | | | | colocalization | Sp076690_GB_1F | GCGCCGTCTCGCTCGAATGGCAAC |
| | | | | | | | TTCTCACATTCGC |
| 66 | | | | | | Sp076690_GB_CT | GCGCCGTCTCGCTCACGAACCTAAC |
| | | | | | | R | CATCCCTTAACAACAGG |
| 67 | | | | | mutagenesis | Spo12715_M1_F | CGTATGAGTGCTTGTTGGAGGATAC |
| | | | | | | | ATTCACTGGATATATG |
| 68 | | | | | | Spo12715_M1_R | CATATATCCAGTGAATGTATCCTCCA |
| | | | | | | | ACAAGCACTCATACG |
| 69 | | | | | | Spo12715_M2_F | CTACGGTTCAACTAATAAGATGGAC |
| | | | | | | | CTCCTCATTACTTGG |
| 70 | | | | | | Spo12715_M2_R | CCAAGTAATGAGGAGGTCCATCTTA |
| | | | | | | | TTAGTTGAACCGTAG |
| 71 | SoGT1 | UGT79M1 | Spinacia | Sp_049940_mmut | VIGS | Sp049940_vF | CTAGAGCTCAGCTTTCCTCCCATGG |
| | | | oleracea | | | | CATC |
| 72 | | | | | | Sp049940_vR | TTCGAGCTCACTTGTGGCTGGAGTT |
| | | | | | | | GAGG |
| 73 | SoGT2 | UGT79N1 | Spinacia | Sp_148240_fnwi | VIGS | Sp148240_vF | CTAGAGCTCCCCTCCTCGGCTATAC |
| | | | oleracea | | | | ACCT |
| 74 | | | | | | Sp148240_vR | TTCGAGCTCCCTACGTCTGCCACAG |
| | | | | | | | TCTC |
| 75 | SOAP6 | UGT74BB2 | Spinacia | Sp_170930_hjgq | VIGS | Sp170930vF | CTAGAGCTCCCGAAGCCTCACTCCA |
| | | | oleracea | | | | ACTT |
| 76 | | | | | | Sp170930vR | TTCGAGCTCTCAGAACTCGGGGATT |
| | | | | | | | GTGC |
| 77 | | | | | expression in N. | Sp170930_GB_1F | GCGCCGTCTCGCTCGAATGACGGG |
| | | | | | benthamiana | | AAAAGGAAGAACG |
| 78 | | | | | | Sp170930_GB_1R | GCGCCGTCTCGCTCAAAGCTTAGGA |
| | | | | | | | GGACGCAAGCCAGT |
| 79 | SOAP6v2 | UGT74BB1 | Spinacia | Sp_170920_oudh | VIGS | Sp170920_vF | CTAGAGCTCTCGTGGCTCGTATGGG |
| | | | oleracea | | | | AAAG |
| 80 | | | | | | Sp170920_vR | TTCGAGCTCAGGAACAGATTCAGCC |
| | | | | | | | GCAA |
| 81 | SOAP7 | UGT79K1 | Spinacia | Sp_020820_yeau | VIGS | Sp020820_vF | CTAGAGCTCTAGCCACAAAGCTGGG |
| | | | oleracea | | | | GATG |
| 82 | | | | | | Sp020820_vR | TTCGAGCTCGGGAGGAATTCAACCT |
| | | | | | | | CGGG |
| 83 | | | | | expression in N. | Sp020820_GB_1F | GCGCCGTCTCGCTCGAATGGGTAA |
| | | | | | benthamiana | | AACAGTAGCAGCT |
| 84 | | | | | | Sp020820_GB_1R | GCGCCGTCTCGCTCAAAGCTTAATT |
| | | | | | | | TGCAGTAAGAAAGCGTTTC |
| 85 | SOAP8 | UGT79L2 | Spinacia | Sp_113700_suxh | VIGS | Sp113700_vF | CTAGAGCTCGTGATGTTCCCATGGC |
| | | | oleracea | | | | TTGC |
| 86 | | | | | | Sp113700_vR | TTCGAGCTCAAGGTCGGGCTTATGG |
| | | | | | | | GTTG |
| 87 | | | | | expression in N. | Sp113700_GB_1F | GCGCCGTCTCGCTCGAATGGGTGG |
| | | | | | benthamiana | | AGAGAAAGAGTTG |
| 88 | | | | | | Sp113700_GB_1R | GCGCCGTCTCGCTCAAAGCCTAAG |
| | | | | | | | GAACAAGGGCTTGTAA |
| 89 | SOAP8v2 | UGT79L1 | Spinacia | Sp_072870_funr | VIGS | Sp072870_vF | CTAGAGCTCGGCTTGCCTTTGGACA |
| | | | oleracea | | | | CTTG |
| 90 | | | | | | Sp072870_vR | TTCGAGCTCAAGACAAGGTCGGGCT |
| | | | | | | | TCTG |
| 91 | SOAP9 | UGT73BS1 | Spinacia | Sp_170320_dmqi | VIGS | Sp170320_vF | CTAGAGCTCGTGTGCTGCTGAGGTT |
| | | | oleracea | | | | GTTG |
| 92 | | | | | | Sp170320_vR | TTCGAGCTCGTCGCAGTGTACCGGA |
| | | | | | | | TAGG |
| 93 | | | | | expression in N. | Sp170320_GB_1F | GCGCCGTCTCGCTCGAATGGAGCT |
| | | | | | benthamiana | | TTCAAACCCTAGC |
| 94 | | | | | | Sp170320_GB_1R | GCGCCGTCTCGGCGACGAACCACC |
| | | | | | | | TTCTTCA |
| 95 | | | | | | Sp170320_GB_2F | GCGCCGTCTCGTCGCGTAACAATTT |
| | | | | | | | AAGTGATTTG |
| 96 | | | | | | Sp170320_GB_2R | GCGCCGTCTCGCTCAAAGCTTATTC |
| | | | | | | | TGAGTTTGTGGACACTG |
| 97 | BvCSLG | CSLG2 | Beta | XM_010673823.2 | VIGS | BvCSL_vF | AAAAGAGCTCCCTTCGGCTCCTCCA |
| | | | vulgaris | | | | CAAAA |
| 98 | | | | | | BvCSL_vR | AAAAGAGCTCACGAAACTCCGAAAA |
| | | | | | | | GGGCT |
| 99 | | | | | expression in N. | BvCSL_GB_1F | GCGCCGTCTCGCTCGAATGTCTTCT |
| | | | | | benthamiana | | CTCCACATTTGC |
| 100 | | | | | | BvCSL_GB_1R | GCGCCGTCTCGGCCTCTTTTCCCTG |
| | | | | | | | GATACG |
| 101 | | | | | | BvCSL_GB_2F | GCGCCGTCTCGAGGCCAAATCGTC |
| | | | | | | | CTCATCG |
| 102 | | | | | | BvCSL_GB_2R | GCGCCGTCTCGCATCTCTTGTCATT |
| | | | | | | | TCCTTCAAG |
| 103 | | | | | | BvCSL_GB_3F | GCGCCGTCTCGGATGACTTGTTAGA |
| | | | | | | | AGAGGCT |
| 104 | | | | | | BvCSL_GB_3R | GCGCCGTCTCGCTCAAAGCTCAATC |
| | | | | | | | ACGTCCTTTTCTTACTTT |
| 105 | CqCSLG | CSLG2 | Chenopodi | XM_021866098.1 | expression in N. | CqCSL_GB_1F | GCGCCGTCTCGCTCGAATGGCGGC |
| | | | um quinoa | | benthamiana | | AACACACATTTG |
| 106 | | | | | | CqCSL_GB_1R | GCGCCGTCTCGGCCTCTTTTCCCTG |
| | | | | | | | GCTACA |
| 107 | | | | | | CqCSL_GB_2F | GCGCCGTCTCGAGGCCAGGTCATC |
| | | | | | | | CTCATCG |
| 108 | | | | | | CqCSL_GB_2R | GCGCCGTCTCGCTCAAAGCTTATTC |
| | | | | | | | TTTCTTTCTAAGTTTGTCTG |
| 109 | MsCSLG | CSLG2 | Medicago | MSAD 299835 | expression in N. | MsCSL_GB_1F | GCGCCGTCTCGCTCGAATGGCAAC |
| | | | sativa | | benthamiana | | CTTCACATTTCAC |
| 110 | | | | | | MsCSL_GB_1R | GCGCCGTCTCGCATCTCTTGAAATA |
| | | | | | | | TTCTGCTTCT |
| 111 | | | | | | MsCSL_GB_2F | GCGCCGTCTCGGATGTAATTTTACA |
| | | | | | | | AGAAGCATGTG |
| 112 | | | | | | MsCSL_GB 2R | GCGCCGTCTCGCTCAAAGCCTACC |
| | | | | | | | CACTCTTCCGCTTCA |
| 113 | | | | | silencing in M. sativa | MsCSL_asGB_1F | GCGCCGTCTCGCTCGAATGCTACC |
| | | | | | hairy roots | | CACTCTTCCGCTTCA |
| 114 | | | | | | MsCSL_asGB_1R | GCGCCGTCTCGCTCAAAGCATGGC |
| | | | | | | | AACCTTCACATTTCAC |
| 115 | GmCSL | CSLG2 | Glycine | NM_001365113.1 | expression in N. | GmCSL_GB_1F | GCGCCGTCTCGCTCGAATGGCGAC |
| | | | max | | benthamiana | | CTTCCACACAGA |
| 116 | | | | | | GmCSL_GB_1R | GCGCCGTCTCGGGATCTCTTCCAAA |
| | | | | | | | TTCGTCA |
| 117 | | | | | | GmCSL_GB_2F | GCGCCGTCTCGATCCTAAAAATCGT |
| | | | | | | | TCCATTGTGT |
| 118 | | | | | | GmCSL_GB_2R | GCGCCGTCTCGCTCAAAGCCTATTG |
| | | | | | | | CACCTTGCTTTTCATG |
| 119 | GuCSL | CSLG2 | Glycyrrhiza | Glyur003152s0003749 | expression in N. | GuCSL_GB_1F | GCGCCGTCTCGCTCGAATGGCAAG |
| | | | uralensis | 1.1 | benthamiana | | CTTCACCCTTCA |
| 120 | | | | | | GuCSL_GB_1R | GCGCCGTCTCGATACCAGAGTGTTC |
| | | | | | | | ATCACCT |
| 121 | | | | | | GuCSL_GB_2F | GCGCCGTCTCGGTATCTGCCCTTGC |
| | | | | | | | CATGGA |
| 122 | | | | | | GuCSL_GB_2R | GCGCCGTCTCGAGATCTCGCTCTGA |
| | | | | | | | CCTGAA |
| 123 | | | | | | GuCSL_GB_3F | GCGCCGTCTCGATCTCATCAAGGCT |
| | | | | | | | AAATACGAG |
| 124 | | | | | | GuCSL_GB_3R | GCGCCGTCTCGCTCAAAGCCTATCC |
| | | | | | | | ACTCTTGCTTTTCATG |
| 125 | GuUGAT | UGAT | Glycyrrhiza | KT759000.1 | expression in N. | GuUGAT_GB_1F | GCGCCGTCTCGCTCGAATGACCAT |
| | | | uralensis | | benthamiana | | GGGTAACGAGAAT |
| 126 | | | | | | GuUGAT_GB_1R | GCGCCGTCTCGGCGACGATCCTCC |
| | | | | | | | TTCCTCC |
| 127 | | | | | | GuUGAT_GB_2F | GCGCCGTCTCGTCGCACAACGATTT |
| | | | | | | | TAACTCTTTA |
| 128 | | | | | | GuUGAT_GB_2R | GCGCCGTCTCGCTCAAAGCTTAATG |
| | | | | | | | GGCACGCGACCTCA |
| 129\|2 | GuUGT73P1 | UGT73P12 | Glycyrrhiza | Scaffold00629 | expression in N. | GuUGT73P12_GB_ | GCGCCGTCTCGCTCGAATGGACTC |
| | | | uralensis | (LC314779) | benthamiana | 1F | CTTTGGGGTTGA |
| 130 | | | | | | GuUGT73P12_GB_ | GCGCCGTCTCGGCCTCATTTCACG |
| | | | | | | 1R | GAACAGT |
| 131 | | | | | | GuUGT73P12_GB_ | GCGCCGTCTCGAGGCCAGATTTCAT |
| | | | | | | 2F | AGTCACT |
| 132 | | | | | | GuUGT73P12_GB_ | GCGCCGTCTCGCTCAAAGCTTAAGC |
| | | | | | | 2R | CACTGCCTCCATTAA |
| 133 | ACYLTRANSFERASES | | | | | | |
| 134 | SoAT1 | uncharacterized | Spinacia | Sp_074630_ygho | VIGS | Spo04549_vF | CTAGAGCTCAGGCGTTGCTATCGAT |
| | | acetyltransferase | oleracea | | | | CCAG |
| | | At3g50280-like | | | | | |
| 135 | | | | | | Spo04549_vR | TTCGAGCTCCATGGCCTTGAGTCTC |
| | | | | | | | AGCA |
| 136 | SoAT2 | O- | Spinacia | Sp_123780_pgiy | VIGS | Spo21561_vF | CTAGAGCTCCCGGAAATGAAGGTCT |
| | | acyltransferase | oleracea | | | | GGGT |
| | | WSD1-like | | | | | |
| 137 | | | | | | Spo21561_vR | TTCGAGCTCAGGGGGTTCGAGCATT |
| | | | | | | | TGTC |
| 138 | SoAT3 | acyl-CoA--sterol | Spinacia | Sp_149180_nwmy | VIGS | Spo15788_vF | CTAGAGCTCTGATAAGGGCCCACTT |
| | | O- | oleracea | | | | TGCG |
| | | acyltransferase 1 | | | | | |
| 139 | | | | | | Spo15788_vR | TTCGAGCTCCTCCAGTTCGAGCCCT |
| | | | | | | | AACAA |
| 140 | SoAT4 | malonyl- | Spinacia | Sp_198340_focw | VIGS | Spo13090_vF | CTAGAGCTCAAGCCAAAGCAAAAGG |
| | | CoA:anthocyanid | oleracea | | | | CACC |
| | | in 5-O-glucoside- | | | | | |
| | | 6″-O- | | | | | |
| | | malonyltransfera | | | | | |
| | | se-like | | | | | |
| 141 | | | | | | Spo13090_vR | TTCGAGCTCTCGGTTTCCCCCACCA |
| | | | | | | | AAAA |
| 142 | SOAP10 | salutaridinol 7-O- | Spinacia | Sp_125800_kzws | VIGS | Spo02253_vF | CTAGAGCTCCAACAACTTCCAAAGG |
| | | acetyltransferase | oleracea | | | | CGGC |
| 143 | | | | | | Spo02253_vR | TTCGAGCTCTCTTGAAGCCCATTGC |
| | | | | | | | TGCT |
| 144 | | | | | expression in | Spo02253_NdeI_F | GAAACATATGGGGGAAGTCAACCAT |
| | | | | | E. coli | | GAAGAAG |
| 145 | | | | | | Spo02253_NotI_R | CTTTGCGGCCGCCTAATTAGGAGTA |
| | | | | | | | GCAAAAGCAAGG |
| 146 | | | | | expression in | Spo02253_GB_1F | GCGCCGTCTCGCTCGAATGGGGGA |
| | | | | | N. benthamiana | | AGTCAACCATGA |
| 147 | | | | | | Spo02253_GB_1R | GCGCCGTCTCGCTGTCTCAAATTTG |
| | | | | | | | GTGGGTT |
| 148 | | | | | | Spo02253_GB_2F | GCGCCGTCTCGACAGTGACAGGGT |
| | | | | | | | TTATTTGG |
| 149 | | | | | | Spo02253_GB_2R | GCGCCGTCTCGGCCTCCCATCGTT |
| | | | | | | | GGGAATT |
| 150 | | | | | | Spo02253_GB_3F | GCGCCGTCTCGAGGCCCCCTAAGG |
| | | | | | | | TCAGGAA |
| 151 | | | | | | Spo02253_GB_3R | GCGCCGTCTCGCTCAAAGCCTAATT |
| | | | | | | | AGGAGTAGCAAAAGCAAG |
| 152 | | | | OTHERS | | | |
| 153 | SoCHLH | Magnesium | Spinacia | Sp_082000_pjxp | VIGS | SoCHLH_vF | ACTAGAATTCCAAGTGGGGATGAGT |
| | | chelatase | oleracea | | | | GATGCTTG |
| | | subunit H | | | | | |
| 154 | | | | | | SoCHLH_VR | GTTCGAATTCCTAGCAGTGCTGATG |
| | | | | | | | ATGGAACTC |
| 155 | BvCHLH | Magnesium | Beta | XM_010674548.2 | VIGS | BvCHLH_vF | AAAAGAATTCGGAGGCAAGAGGGG |
| | | chelatase | vulgaris | | | | CTAAAG |
| | | subunit H | | | | | |
| 156 | | | | | | BvCHLH_vR | AAAAGAATTCTTTCCTCTTCAAGTCC |
| | | | | | | | GCCC |
| 157 | SoUGD1 | UDP-glucose 6- | Spinacia | Sp_189830_psca | expression in yeast | SoUGD1_SalI_F | AAAAGTCGACATGGTGAAGAAACTG |
| | | dehydrogenase | oleracea | | | | AAGATTTGCTG |
| | | 1 | | | | | |
| 158 | | | | | | SoUGD1_XhoI_R | AAAACTCGAGTTAAGCTACGGCAGG |
| | | | | | | | CATGTC |

Transient Expression and Candidate Gene Screening in N. benthamiana

pDBG3α and pDBG3Ω constructs were transformed into Agrobacterium tumafaciens (GV3101) electrocompetent cells. Transformants were grown on LB plates containing 50 μg/mL kanamycin (pDBG3α) or 200 μg/mL spectinomycin (pDBG3Ω) and 50 μg/mL gentamicin at 28° C. 10 mL of LB medium supplemented with antibiotics was inoculated with a single colony and grew O/N at 28° C. Cells were centrifuged at 3,000×g for 10 min. and supernatant removed. Pellet was resuspended in 5 ml of infiltration medium (100 mM MES buffer, 2 mM Na₃PO₄·12H₂O, 100 μM acetosyringone) and centrifuged again. Pellet was resuspended again in 10 mL of infiltration medium and incubated at room temperature for 2 h. Agrobacterium suspensions (OD600=0.3 for each strain) were infiltrated into the underside of N. benthamiana leaves with a needleless 1 mL syringe. Plants were grown 4-5 weeks under a 16 h light cycle prior to infiltration. Leaves were harvested 4 days post-infiltration, frozen in liquid nitrogen and stored at −80° C. for later processing. Biological replicates consisted of several leaves all from different tobacco plants.

Expression of SOAP Genes in S. cerevisiae WAT 11 and Metabolite Extraction

pESC constructs were transformed into Saccharomyces cerevisiae WAT11 using Yeastmaker™ yeast transformation system (Clontech). Yeast cells were transformed with various combinations of pESC vectors allowing expression of one (SOAP5), two (SOAP1-2), four (SOAP1-4), five (SOAP1-5 or SOAP1-4+SoUGD1) and 6 genes (SOAP1-5+SoUGD1). Transformed yeast were grown on SD minimal media supplemented with appropriate amino acids and 2% glucose. Colonies were screened and presence of transgene confirmed by colony PCR. For induction of gene expression transformed cells were transferred to minimal medium with 2% galactose and grown for 24h at 30° C. Cultures were centrifuged for 10 min. at 700×g, pellet was resuspended in 1 mL of H₂O, transferred to 2 mL Eppendorf tube and centrifuged again at 8,000×g for a minute. Cell pellet was weighed, equal amount of water and double amount of glass beads (diameter 500 μm) was added and vortexed 5 times for one minute, each time keeping the cells on ice for one minute between vortexings. Lysed cells were mixed with 500 μL of methanol and centrifuged at 14′000 rpm for 5 minutes, clear supernatant was collected and dried in the SpeedVac O/N. Dry residues were dissolved in 150 μL of 80% methanol, filtered through 0.22 μm filter and analyzed on LC-MS.

### Subcellular Localization and Confocal Microscopy Analysis

Plasmids allowing expression of studied proteins in fusion with fluorescent protein (FP) were prepared using GoldenBraid system (M. Vazquez-Vilar et al., (2017) ibid). SOAP1:GFP, SOAPs 2-4 in fusion with -GFP or -RFP and SOAP5:RFP were used. As cell compartment markers plasmids obtained from ABRC (ER-gk CD3-955 and G-gk CD3-963) were used (B. K. Nelson, X. Cai, A. Nebenführ, A multicolored set of in vivo organelle markers for co-localization studies in Arabidopsis and other plants. Plant J. 51, 1126-36 (2007)).

For subcellular localization studies of spinach cellulose synthase like G, SOAP5:RFP was transiently expressed together with ER or Golgi marker in N. benthamiana epidermal cells. A bacterial absorbance A600 nm of 0.15 was used for infiltrating each Agrobacterium strain. After 72h post infiltration, leaf discs (˜0.4 cm diameter) were collected and analyzed for fluorescence with confocal microscopy using the following parameters: Fluorescence was observed by a Nikon eclipse Al microscope with laser at 488 nm for excitation and images were acquired for GFP (ER or Golgi marker) and 561 nm for red fluorescent protein (RFP) signals. To increase signal-to-noise ratio, each scan pixel was sampled four times and averaged. The same parameters were used for checking colocalization of SOAP genes.

### Fluorescence Resonance Energy Transfer (FRET) Analysis

Mean FRET index (i.e., the mean of acceptor, mRFP-fused protein, intensity due to FRET in each pixel after threshold application to remove background noise) were calculated using the FRET Analyzer plugin (M. Hachet-Haas et al., FRET and colocalization analyzer a method to validate measurements of sensitized emission FRET acquired by confocal microscopy and available as an ImageJ Plug-in. Microsc Res Tech. 69, 941-56 (2006)) FIJI/ImageJ. The mean FRET index was calculated for three independent images for each protein combination being tested for proximity/interaction. Cells expressing GFP and RFP only were used to calculate donor and acceptor bleed through.

### Phylogenetic Analysis

Homology searches were performed with various query sequences at the non-redundant protein database of NCBI (http://blast.ncbi.nlm.nih.gov/Blast.cgi) using the Blastp option. Protein sequences were aligned using the Muscle algorithm and phylogenetic tree was inferred with RAxML rapid bootstrapping and subsequent ML search (A. Stamatakis, RAxML version 8: a tool for phylogenetic analysis and post-analysis of large phylogenies. Bioinformatics. 30, 1312-3 (2014)). Likelihood of final tree was evaluated and optimized under GAMMA model of rate heterogeneity across sites. GAMMA model parameters were estimated up to an accuracy of 0.1000000000 Log Likelihood units. One thousand bootstrap replicates using the fast bootstrap option of RAxML were performed. Used substitution matrix was DAYHOFF. Phylogenetic tree was visualized with iTOL (I. Letunic, P. Bork, Interactive tree of life (iTOL) v3: an online tool for the display and annotation of phylogenetic and other trees. Nucleic Acids Res. 44, W242-5 (2016)).

Silencing of MsCSL in M. sativa Hairy Roots

Vector pDBG3α2 with antisense RNA targeting MsCSL under 35S promoter and with 35S:KanR cassette was generated using GoldenBraid system. Transformation of M. sativa and generation of transgenic hairy roots was performed as described previously (A. Boisson-Dernier, M. Chabaud, F. Garcia, G. Bëcard, C. Rosenberg, D. G. Barker, Agrobacterium rhizogenes-transformed roots of Medicago truncatula for the study of nitrogen-fixing and endomycorrhizal symbiotic associations. Mol Plant Microbe Interact. 14, 695-700 (2001)). Instead of Arqual, ATCC15834 strain of A. rhizogenes was used. Transgenic roots were collected, ground and metabolites extracted as described above.

### Template-Based Protein Structure Modeling

A homology model of SOAP5 was generated with RaptorX (M. Källberg et al., Template-based protein structure modeling using the RaptorX web server. Nature Protocols. 7, 1511-1522 (2012)) using the crystal structure of Rhodobacter sphaeroides cellulose synthase in complex with cyclic-di-GMP and UDP as a template (PDB entry: 4P00) (J. L. Morgan, J. T. McNamara, J. Zimmer, Mechanism of activation of bacterial cellulose synthase by cyclic di-GMP. Nat Struct Mol Biol. 21, 489-96 (2014)). Best model was used as a template for MOLE 2.5, a universal toolkit for automated location and characterization of channels, tunnels and pores (L. Pravda et al., MOLEonline: a web-based tool for analyzing channels, tunnels and pores (2018 update). Nucleic Acids Res. 46, W368-W373 (2018)). Scheme representing transmembrane topology of SOAP5 was generated with Protter (U. Omasits, C. H. Ahrens, S. Müller, B. Wollscheid, Protter: interactive protein feature visualization and integration with experimental proteomic data. Bioinformatics. 30, 884-6 (2014)).

### Example 15: Triterpenoid Saponins Biosynthesis Pathway in Spinach

Objective: To discover the triterpenoid saponins biosynthesis pathway in spinach (Spinacia oleracea), member of the Caryophyllales, particularly enzymes involved in the glycosylation of triterpenoid saponins.

Methods: Liquid chromatography-Mass spectroscopy methods were used in the analysis of saponins in spinach leaves. See Materials and Methods above.

### Results:

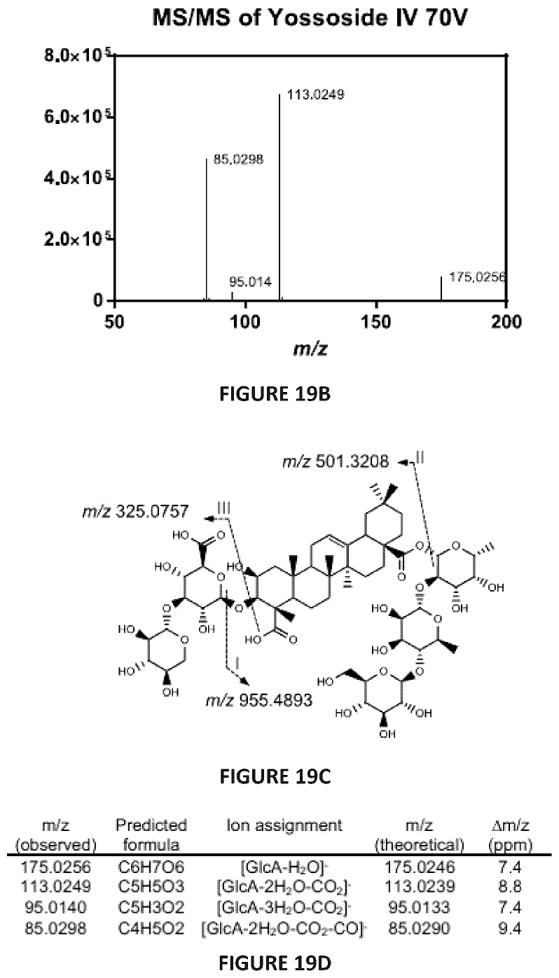

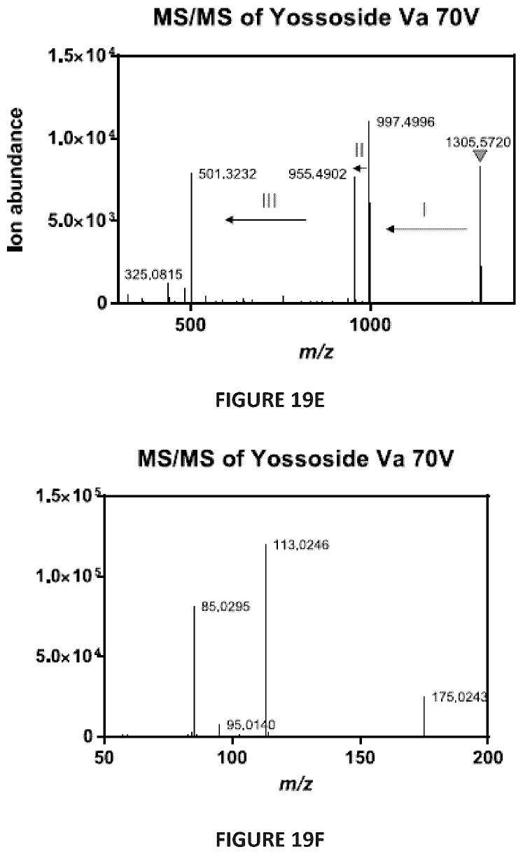

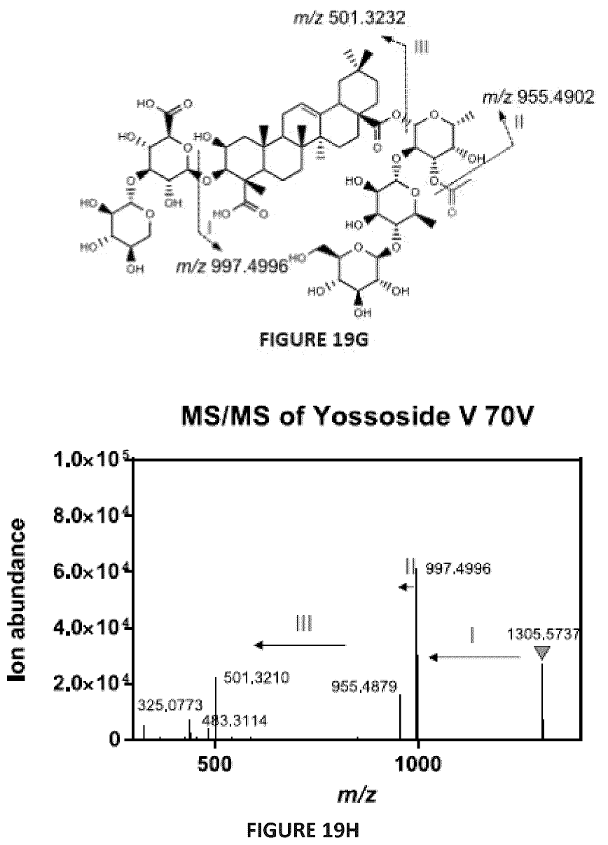

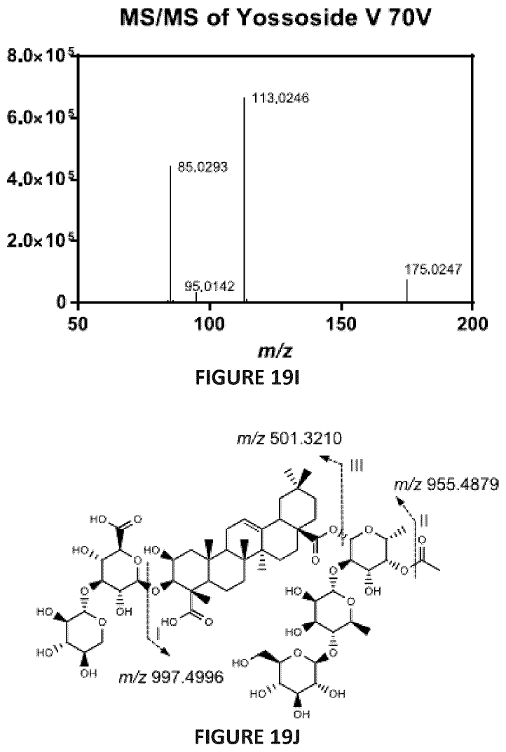

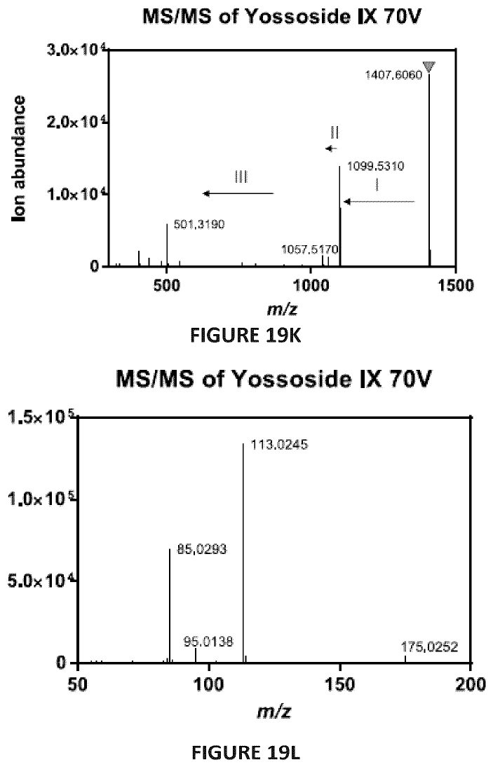

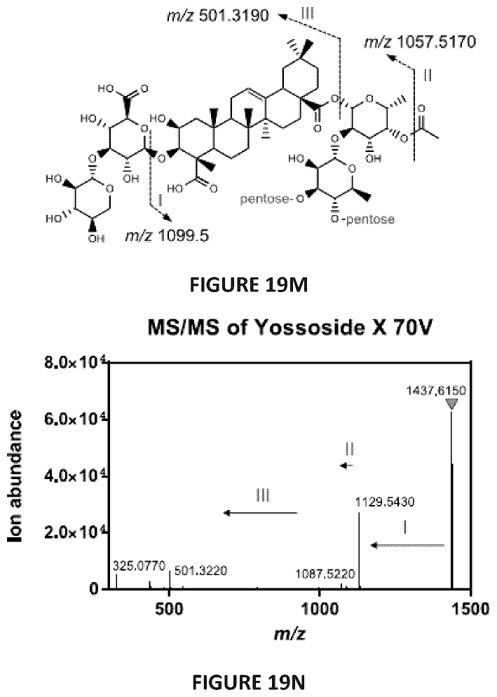

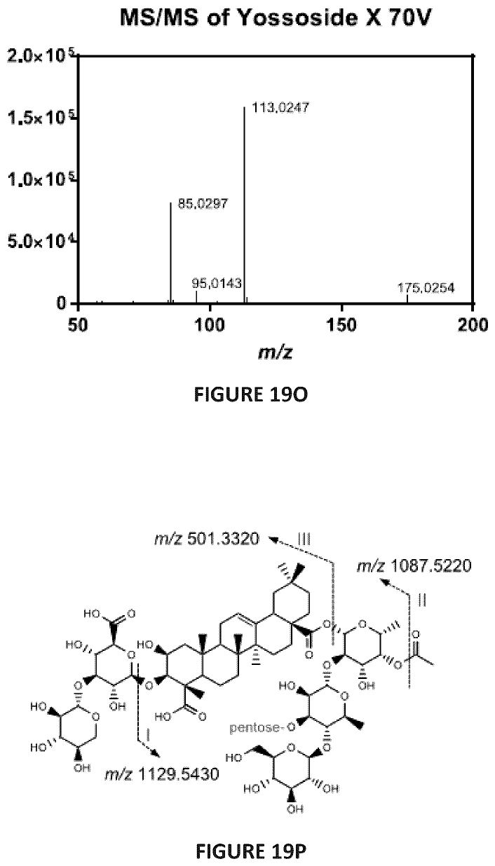

Analysis of spinach leaves (using LC-MS) revealed a complex triterpenoid saponin mixture (FIG. 18, FIGS. 19A-19P, Table 12) comprising more than 20 molecules with medicagenic acid as an aglycone and approximately half of them were acetylated. Table 13 below provides the details for each compound identified by number in the lower scan of FIG. 18. Table 12 below uses the following terms: Sample—type of experiment/genotype that sample was derived from; Ret. Time—Retention time, in minutes; Putative Name—putative metabolite identification; Mol. Formula—molecular formula of the metabolite or its FA adduct; Theor. m/z—theoretical monoisotopic mass calculated for the ion [M−H]-, [M+H]+; Found m/z—mass found; m/z error (ppm)—difference between theoretical and found m/z values in ppm; MS/MS fragments—fragments, obtained from the ion [M−H]−, [M+H]+; MS/MS CE (eV)—collision energy used for fragmentation; UV/Vis—UV/Vis absorbance maxima. The UV/Vis spectra (200-600 nm) were acquired on a UPLC (Waters, Acquity) instrument equipped with an Acquity 2996 PDA detector under LC conditions as described in the Materials and Methods. (S)—identification confirmed by the standard compound; (NMR)—identification confirmed by NMR; MA—medicagenic acid; AA—augustic acid (2-hydroxy oleanolic acid); B—bayogenin; PG—polygalagenin; OA—oleanolic acid; G—gypsogenin; GA—gypsogenic acid; H—hederagenin; GhA—glycyrrhetinic acid; hexA—hexuronic acid; hex—hexose; dhex—deoxyhexose; pent—pentose; GlcA—glucuronic acid; Ac—acetyl.

TABLE 12
Mass Spectroscopy based identification of triterpenoid saponins in studied plants.
Section A

| | | | C | | | | |
|---|---|---|---|---|---|---|---|
| | | | combination | | | | |
| | | | of genes | D | | | G |
| | | B | expressed in | Ret. | E | F | Mol |
| 1 | A | Sample | N. benthamiana | Time | Putative Name | Aglycone + sugars | formula |
| 2 | Spinach | WT | — | 13.7 | | MA + dhex + dhex + | C53 H82 |
| | | | | | | pent + hexA | O24 |
| 3 | Spinach | | — | 12.5 | Yossoside VI | MA + dhex + dhex + | C54 H84 |
| | | | | | | hex + hexA | O25 |
| 4 | Spinach | | — | 19.4 | | MA + dhex + dhex + | C55 H84 |
| | | | | | | Ac + pent + hexA | O25 |
| 5 | Spinach | | — | 14.7 | Yossoside XII | MA + dhex + dhex + | C58 H90 |
| | | | | | | pent + pent + hexA | O28 |
| 6 | Spinach | | — | 12.8 | Yossoside IV | MA + dhex + dhex + | C59 H92 |
| | | | | | | hex + pent + hexA | O29 |
| 7 | Spinach | | — | 20.84 | Yossoside VII | MA + dhex + dhex + | C60 H92 |
| | | | | | | Ac + pent + pent + | O29 |
| | | | | | | hexA | |
| 8 | Spinach | | — | 15.8 | Yossoside Va | MA + dhex + dhex + | C61 H94 |
| | | | | | | Ac + hex + pent + | O30 |
| | | | | | | hexA | |
| 9 | Spinach | | — | 18.6 | Yossoside V (NMR) | MA + dhex + dhex + | C61 H94 |
| | | | | | | Ac + hex + pent + | O30 |
| | | | | | | hexA | |
| 10 | Spinach | | — | 14.2 | Yossoside VIII | MA + dhex + dhex + | C63 H98 |
| | | | | | | pent + pent + pent | O32 |
| | | | | | | + hexA | |
| 11 | Spinach | | — | 12.4 | Yossoside XI | MA + dhex + dhex + | C64 H100 |
| | | | | | | hex + pent + pent + | O33 |
| | | | | | | hexA | |
| 12 | Spinach | | — | 18.9 | Yossoside IX | MA + dhex + dhex + | C65 H100 |
| | | | | | | Ac + pent + pent + | O33 |
| | | | | | | pent + hexA | |
| 13 | Spinach | | — | 16.7 | Yossoside X | MA + dhex + dhex + | C66 H102 |
| | | | | | | Ac + hex + pent + | O34 |
| | | | | | | pent + hexA | |
| 14 | | | | | | | |
| 15 | Spinach | VIGS -CYPs | — | 16.6 | | AA + hex + hexA | C42 H66 |
| | | | | | | | O15 |
| 16 | Spinach | | — | 10.3 | | B + hex + hexA | C42 H66 |
| | | | | | | | O16 |
| 17 | Spinach | | — | 15.4 | | AA + hex + pent + | C47 H74 |
| | | | | | | hexA | O19 |
| 18 | Spinach | | — | 15.4 | | AA + hex + hexA + | C47 H70 |
| | | | | | | 3-oxopyruvic acid | O21 |
| | | | | | | and glycolic acid | |
| | | | | | | isomer 1 | |
| 19 | Spinach | | — | 16.2 | | AA + hex + hexA + | C47 H70 |
| | | | | | | 3-oxopyruvic acid | O21 |
| | | | | | | and glycolic acid | |
| | | | | | | isomer 2 | |
| 20 | Spinach | | — | 13.1 | | AA + hex + hex + | C48 H76 |
| | | | | | | hexA | O20 |
| 21 | Spinach | | — | 14.4 | | AA + hex + pent + | C52 H82 |
| | | | | | | pent + hexA | O23 |
| 22 | Spinach | | — | 11.6 | | PG + hex + pent + | C52 H80 |
| | | | | | | pent + hexA | O24 |
| 23 | Spinach | | — | 18.7 | Yossoside VIIa | MA + dhex + dhex + | C60 H92 |
| | | | | | | Ac + pent + pent + | O29 |
| | | | | | | hexA | |
| 24 | Spinach | | — | 17.2 | | OA + hex + hexA + | C47 H70 |
| | | | | | | 3-oxopyruvic acid | O20 |
| | | | | | | and glycolic acid | |
| | | | | | | (Betavulgaroside I) | |
| | | | | | | isomer 1 | |
| 25 | Spinach | | — | 18.4 | | OA + hex + hexA + | C47 H70 |
| | | | | | | 3-oxopyruvic acid | O20 |
| | | | | | | and glycolic acid | |
| | | | | | | (Betavulgaroside I) | |
| | | | | | | isomer 2 | |
| 26 | Spinach | | — | 14.6 | | G + hex + hexA + | C47 H68 |
| | | | | | | 3-oxopyruvic acid | O21 |
| | | | | | | and glycolic acid | |
| | | | | | | (Basellasaponin B) | |
| 27 | Spinach | | — | 16.1 | | GA + hex + pent + | C59 H92 |
| | | | | | | dhex + dhex + hexA | O28 |
| 28 | Spinach | | — | 21.2 | | GA + hex + pent + | C61 H94 |
| | | | | | | dhex + dhex + Ac + | O29 |
| | | | | | | hexA | |
| 29 | | | | | | | |
| 30 | Spinach | VIGS − | — | 10.74 | | MA + hex + hexA + | C47 H68 |
| | | GTS + CSL | | | | 3-oxopyruvic acid | O23 |
| | | | | | | and glycolic acid | |
| | | | | | | isomer 1 | |
| 31 | Spinach | | — | 11.90 | | MA + hex + hexA + | C47 H68 |
| | | | | | | 3-oxopyruvic acid | O23 |
| | | | | | | and glycolic acid | |
| | | | | | | isomer 2 | |
| 32 | Spinach | | — | 15.30 | | MA + dhex + hexA + | C47 H68 |
| | | | | | | 3-oxopyruvic acid | O22 |
| | | | | | | and glycolic acid | |
| 33 | Spinach | | — | 15.31 | | MA + hexA + dhex + | C47 H72 |
| | | | | | | pent (Betavulgaroside | O20 |
| | | | | | | III) | |
| 34 | Spinach | | — | 11.00 | | MA + pent + hex + | C47 H72 |
| | | | | | | hexA | O21 |
| 35 | Spinach | | — | 13.40 | | MA + dhex + dhex + | C48 H74 |
| | | | | | | hexA | O20 |
| 36 | Spinach | | — | 17.10 | | OA + hexA + hex + | C47 H74 |
| | | | | | | pent | O18 |
| 37 | Spinach | | — | 12.50 | | AA/H + hexA + hex | C42 H66 |
| | | | | | | | O15 |
| 38 | Spinach | | — | 14.62 | | G + hexA + hex | C42 H64 |
| | | | | | | | O15 |
| 39 | Spinach | | — | 12.90 | | GA/PG + hex + hexA | C42 H64 |
| | | | | | | | O16 |
| 40 | Spinach | | — | 10.00 | | MA + dhex + pent + | C58 H90 |
| | | | | | | pent + hex + hexA | O29 |
| 41 | Spinach | | — | 8.50 | | MA + dhex + hex + | C54 H84 |
| | | | | | | hex + hexA | O26 |
| 42 | Spinach | | — | 9.80 | | MA + dhex + pent + | C53 H82 |
| | | | | | | hex + hexA | O25 |
| 43 | Spinach | | — | 11.00 | | MA + hex + hexA | C42 H64 |
| | | | | | | | O17 |
| 44 | Spinach | | — | 21.10 | | MA + pent + hexA | C41 H62 |
| | | | | | | | O16 |
| 45 | Spinach | | — | 15.25 | | PG/GA + hex + hexA | C42 H64 |
| | | | | | | | O16 |
| 46 | Spinach | | — | 13.20 | | MA + dhex + dhex + | C58 H90 |
| | | | | | | pent + pent + hexA | O28 |
| 47 | Spinach | | — | 17.60 | | MA + dhex + dhex + | C60 H92 |
| | | | | | | Ac + pent + pent + | O29 |
| | | | | | | hexA | |
| 48 | Spinach | | — | 11.15 | | MA + hex + hex | C42 H66 |
| | | | | | | | O16 |
| 49 | Spinach | | — | 22.73 | Medicagenic | MA | C30 H46 |
| | | | | | acid (S) | | O6 |
| 50 | | | | | | | |
| 51 | Medicago | Differential | — | 21.61 | Saponin 1 | B + hex + hex + | C48 H76 |
| | sativa | signals form | | | | hexA | O21 |
| 52 | Medicago | M sativa | — | 25.63 | Saponin 2 | B + hex + hexA | C42 H66 |
| | sativa | hairy roots | | | | | O16 |
| 53 | Medicago | with silenced | — | 23.04 | Saponin 3 | Soyasapogenol A + | C48 H78 |
| | sativa | MsCSL | | | | dhex + hex + hexA | O19 |
| 54 | Medicago | | — | 24.11 | Saponin 4 | GA/PG + hex + | C45 H66 |
| | sativa | | | | | hexA + malonyl | O19 |
| 55 | Medicago | | — | 25.23 | Saponin 5 | B + dhex + hex + | C48 H76 |
| | sativa | | | | | hexA | O20 |
| 56 | Medicago | | — | 22.84 | Saponin 6 | H + hex + hex + | C54 H86 |
| | sativa | | | | | dhex + hexA | O24 |
| 57 | Medicago | | — | 22.92 | Saponin 7 | GA/PG + hex + | C42 H64 |
| | sativa | | | | | hexA | O16 |
| 58 | Medicago | | — | 28.14 | Saponin 8 | Soyasapogenol B + | C54 H88 |
| | sativa | | | | | dhex + hex + hex + | O23 |
| | | | | | | hexA | |
| 59 | | | | | | | |
| 60 | Beta | Differential | | 14.95 | BvSaponin 1 | OA + hexA + hex + | C53 H82 |
| | vulgaris | signals from | | | (betavulgaroside V) | hex + Act | O25 |
| 61 | Beta | B. vulgaris | | 15.24 | BvSaponin 2 | OA + hexA + hex + | C53 H84 |
| | vulgaris | with silenced | | | | hex + pent | O23 |
| 62 | Beta | BvCSL | | 15.67 | BvSaponin 3 | OA + hexA + hex + | C52 H80 |
| | vulgaris | | | | (betavulgaroside IX) | pent + Act | O24 |
| 63 | Beta | | | 16.95 | BvSaponin 4 | OA + hex + pent + | C47 H74 |
| | vulgaris | | | | | hexA | O18 |
| 64 | Beta | | | 17.54 | BvSaponin 5 | OA + hexA + hex + | C47 H72 |
| | vulgaris | | | | (betavulgaroside III) | Act | O20 |
| 65 | | | | | | | |
| 66 | Expression in | genes from | SOAP1-4 + | 20.98 | medicagenic | MA + GlcA | C36 H54 |
| | N. benthamiana | spinach | SOAP5 | | acid 3-O- | | O12 |
| | | | | | glucuronide (NMR) | | |
| 67 | Expression in | | SOAP1-5 + | 14.96 | Yossoside I | MA + GlcA + Fuc | C42 H64 |
| | N. benthamiana | | SOAP6 | | | | O16 |
| 68 | Expression in | | SOAP1-6 + | 13.21 | Yossoside II | MA + GlcA + Fuc + | C48 H74 |
| | N. benthamiana | | SOAP7 | | | Rha | O20 |
| 69 | Expression in | | SOAP1-7 + | 12.08 | Yossoside III | MA + GlcA + Fuc + | C54 H84 |
| | N. benthamiana | | SOAP8 | | | Rha + Glc | O25 |
| 70 | Expression in | | SOAP1-8 + | 12.37 | Yossoside IV | MA + GlcA + Fuc + | C59 H92 |
| | N. benthamiana | | SOAP9 | | | Rha + Glc + Xyl | O29 |
| 71 | Expression in | | SOAP1-9 + | 18.18 | Yossoside V | MA + GlcA + Fuc + | C61 H94 |
| | N. benthamiana | | SOAP10 | | | Ac + Rha + Glc + | O30 |
| | | | | | | Xyl | |
| 72 | | | | | | | |
| 73 | Expression in | genes from | bAS + CYP72A154 + | 17.2 | glycyrrhetinic | GhA + GlcA | C36 H54 |
| | N. benthamiana | G. uralensis | CYP88D6 + GuCSL | | acid 3-O- | | O10 |
| | | | | | glucuronide | | |
| 74 | Expression in | | bAS + CYP72A154 + | 19.0 | glycyrrhetinic | GhA + GlcA | C36 H54 |
| | N. benthamiana | | CYP88D6 + GuCSL | | acid 3-O- | | O10 |
| | | | | | glucuronide | | |
| 75 | | | | | | | |
| 76 | Expression in | | bAS + CYP72A154 + | 17.2 | glycyrrhetinic | GhA + GlcA | C36 H54 |
| | N. benthamiana | | CYP88D6 + SOAP5 | | acid 3-O- | | O10 |
| | | | | | glucuronide | | |
| 77 | Expression in | | bAS + CYP72A154 + | 19.0 | glycyrrhetinic | GhA + GlcA | C36 H54 |
| | N. benthamiana | | CYP88D6 + SOAP5 | | acid 3-O- | | O10 |
| | | | | | glucuronide | | |
| 78 | | | | | | | |
| 79 | Expression in | | bAS + CYP72A154 + | 11.8 | glycyrrhizin | GhA + GlcA + GlcA | C42 H62 |
| | N. benthamiana | | CYP88D6 + GuCSL + | | isomer 1 (S) | | O16 |
| | | | UGT73P12 | | | | |
| 80 | Expression in | | bAS + CYP72A154 + | 13.4 | glycyrrhizin | GhA + GlcA + GlcA | C42 H62 |
| | N. benthamiana | | CYP88D6 + GuCSL + | | isomer 2 (S) | | O16 |
| | | | UGT73P12 | | | | |
| 81 | | | | | | | |
| 82 | Expression in | | SOAP1-4 + GuCSL | 14.2 | medicagenic | MA + GlcA | C36 H54 |
| | N. benthamiana | | | | acid 3-O- | | O12 |
| | | | | | glucuronide (S) | | |

Section A

| | H | I | J | K | L | M | N | O |
|---|---|---|---|---|---|---|---|---|
| | ES(+) | ES(+) | m/z | ES(−) | ES(−) | m/z | MS/MS | MS/MS |
| | Theor m/z | Found m/z | error | Theor m/z | Found m/z | error | ES(+) | ES(+) CE |
| 1 | [M + H]⁺ | [M + H]⁺ | (ppm) | [M − H]⁻ | [M − H]⁻ | (ppm) | fragments | (eV) |
| 2 | — | — | — | 1101.5118 | 1101.5116 | −0.2 | — | — |
| 3 | — | — | — | 1131.5223 | 1131.5261 | −1.9 | — | — |
| 4 | — | — | — | 1143.5223 | 1143.5265 | 3.7 | — | — |
| 5 | — | — | — | 1233.5599 | 1233.5554 | 1.1 | — | — |
| 6 | — | — | — | 1263.5646 | 1263.5713 | 0.6 | — | — |
| 7 | — | — | — | 1275.5646 | 1275.5640 | −0.5 | — | — |
| 8 | — | — | — | 1305.5752 | 1305.5773 | 1.6 | — | — |
| 9 | — | — | — | 1305.5752 | 1305.5781 | 2.2 | — | — |
| 10 | — | — | — | 1365.5963 | 1365.5981 | 1.3 | — | — |
| 11 | — | — | — | 1395.6069 | 1395.6068 | −0.1 | — | — |
| 12 | — | — | — | 1407.6069 | 1407.6107 | 2.7 | — | — |
| 13 | — | — | — | 1437.6174 | 1437.6160 | 0.6 | — | — |
| 14 | | | | | | | | |
| 15 | — | — | — | 809.4323 | 809.4337 | 1.7 | — | — |
| 16 | — | — | — | 825.4273 | 825.4278 | 0.6 | — | — |
| 17 | — | — | — | 941.4746 | 941.4737 | −1.0 | — | — |
| 18 | — | — | — | 969.4331 | 969.4326 | −0.5 | — | — |
| 19 | — | — | — | 969.4331 | 969.4327 | −0.4 | — | — |
| 20 | — | — | — | 971.4852 | 971.4866 | 1.4 | — | — |
| 21 | — | — | — | 1073.5169 | 1073.5216 | 4.4 | — | — |
| 22 | — | — | — | 1087.4961 | 1087.4990 | 2.7 | — | — |
| 23 | — | — | — | 1275.5646 | 1275.5675 | 2.3 | — | — |
| 24 | — | — | — | 953.4382 | 953.4366 | −1.7 | — | — |
| 25 | — | — | — | 953.4382 | 953.4369 | −1.4 | — | — |
| 26 | — | — | — | 967.4175 | 967.4183 | 0.8 | — | — |
| 27 | — | — | — | 1247.5697 | 1247.5695 | −0.2 | — | — |
| 28 | — | — | — | 1289.5803 | 1289.5781 | −1.7 | — | — |
| 29 | | | | | | | | |
| 30 | — | — | — | 999.4073 | 999.4097 | 2.4 | — | — |
| 31 | — | — | — | 999.4081 | 999.4097 | 1.6 | — | — |
| 32 | — | — | — | 983.4124 | 983.4155 | 3.2 | — | — |
| 33 | — | — | — | 955.4539 | 955.4543 | 0.4 | — | — |
| 34 | — | — | — | 971.4488 | 971.4506 | 1.9 | — | — |
| 35 | — | — | — | 969.4695 | 969.4661 | −3.5 | — | — |
| 36 | — | — | — | 925.4797 | 925.4789 | −0.9 | — | — |
| 37 | — | — | — | 809.4323 | 809.4335 | 1.5 | — | — |
| 38 | — | — | — | 807.4167 | 807.4156 | −1.4 | — | — |
| 39 | — | — | — | 823.4116 | 823.4129 | 1.6 | — | — |
| 40 | — | — | — | 1249.5490 | 1249.5537 | 3.8 | — | — |
| 41 | — | — | — | 1147.5173 | 1147.5226 | 4.6 | — | — |
| 42 | — | — | — | 1117.5067 | 1117.5078 | 1.0 | — | — |
| 43 | — | — | — | 839.4065 | 839.4077 | 1.4 | — | — |
| 44 | — | — | — | 809.3960 | 809.3938 | −2.7 | — | — |
| 45 | — | — | — | 823.4116 | 823.4132 | 1.9 | — | — |
| 46 | — | — | — | 1233.5540 | 1233.5499 | −3.3 | — | — |
| 47 | — | — | — | 1275.5646 | 1275.5627 | −1.5 | — | — |
| 48 | — | — | — | 825.4273 | 825.4283 | 1.2 | — | — |
| 49 | 503.3373 | 503.3365 | −1.6 | 501.3216 | 501.3217 | 0.2 | see Fig. S19 | 25 |
| 50 | | | | | | | | |
| 51 | — | — | — | 987.4801 | 987.4788 | −1.3 | — | — |
| 52 | — | — | — | 825.4273 | 825.4273 | 0.0 | — | — |
| 53 | — | — | — | 957.5059 | 957.5059 | −0.2 | — | — |
| 54 | — | — | — | 909.4120 | 909.4120 | 0.0 | — | — |
| 55 | — | — | — | 971.4852 | 971.4849 | −0.3 | — | — |
| 56 | — | — | — | 1117.5431 | 1117.5420 | −1.0 | — | — |
| 57 | — | — | — | 823.4116 | 823.4078 | −4.6 | — | — |
| 58 | — | — | — | 1103.5638 | 1103.5635 | −0.3 | — | — |
| 59 | | | | | | | | |
| 60 | — | — | — | 1117.5067 | 1117.5060 | −0.6 | — | — |
| 61 | — | — | — | 1087.5325 | 1087.5316 | −0.8 | — | — |
| 62 | — | — | — | 1087.4961 | 1087.4978 | 1.6 | — | — |
| 63 | — | — | — | 925.4773 | 925.4782 | −1.6 | — | — |
| 64 | — | — | — | 955.4539 | 955.4536 | −0.3 | — | — |
| 65 | | | | | | | | |
| 66 | — | — | — | 677.3543 | 677.3522 | −2.2 | — | — |
| 67 | — | — | — | 823.4116 | 823.4104 | −1.5 | — | — |
| 68 | — | — | — | 969.4695 | 969.4693 | −0.2 | — | — |
| 69 | — | — | — | 1131.5200 | 1131.5199 | −0.1 | — | — |
| 70 | — | — | — | 1263.5646 | 1263.5649 | 0.2 | — | — |
| 71 | — | — | — | 1305.5752 | 1305.5736 | −1.2 | — | — |
| 72 | | | | | | | | |
| 73 | — | — | — | 645.3644 | 645.3644 | 0.1 | — | — |
| 74 | — | — | — | 645.3644 | 645.3636 | 1.2 | — | — |
| 75 | | | | | | | | |
| 76 | — | — | — | 645.3644 | 645.3648 | −0.6 | — | — |
| 77 | — | — | — | 645.3644 | 645.3659 | −2.3 | — | — |
| 78 | | | | | | | | |
| 79 | — | — | — | 821.3965 | 821.3963 | 0.3 | — | — |
| 80 | — | — | — | 821.3965 | 821.3960 | 0.6 | — | — |
| 81 | | | | | | | | |
| 82 | — | — | — | 677.3543 | 677.3535 | 1.0 | — | — |

Section B

| | | B | P |
|---|---|---|---|
| 1 | A | Sample | MS/MS ES(−) fragments |
| 2 | Spinach | WT | 969.4370 [M-pent-H]-; 955.4099 [M-dhex-H]-; 793.4330 [M-hexA-pent-H]-; |
| | | | 501.3204 [M-hexA-pent-dhex-dhex-H]- |
| 3 | Spinach | | 955.4599 [M-hexA-H]-; 823.4120 [M-hex-dhex-H]-; 677.3536 [M-hex-dhex-dhex- |
| | | | H]-; 501.3264 [M-hex-dhex-dhex-hexA-H]- |
| 4 | Spinach | | 1011.4806 [M-pent-H]-; 835.4438 [M-pent-hexA-H]-; |
| 5 | Spinach | | 925.4760 [M-hexA-pent-H]-; 501.3236 [M-hexA-pent-pent-dhex-dhex-H]- |
| 6 | Spinach | | 955.4893 [M-hexA-pent-H]-; 809.3924 [M-hexA-pent-dhex-H]-; 501.3208 [M-hexA- |
| | | | pent-hex-dhex-dhex-H]- |
| 7 | Spinach | | 1143.5160 [M-pent-]-; 1011.4427 [M-pent-pent-H]-; 967.4788 [M-pent-hexA-H]-; |
| | | | 823.4001 [M-pent-pent-dhex-Ac-H]-; 677.3595 [M-pent-pent-dhex-Ac-dhex-H]-; 501.3212 |
| | | | [M-pent-pent-dhex-Ac-dhex-hexA-H]- |
| 8 | Spinach | | 997.4996 [M-hexA-pent-H]-; 955.4902 [M-hexA-pent-Ac-H]-; 501.3232 [M-hexA-pent- |
| | | | dhex-dhex-Ac-hex-H]- |
| 9 | Spinach | | 997.4996 [M-hexA-pent-H]-; 955.4879 [M-hexA-pent-Ac-H]-; 501.3210 [M-hexA-pent- |
| | | | dhex-dhex-Ac-hex-H]- |
| 10 | Spinach | | 1057.5228 [M-hexA-pent-H]-; 925.4678 [M-hexA-pent-pent-H]-; 647.3792 [M-hexA- |
| | | | pent-pent-pent-dhex-H]-; 501.3193 [M-hexA-pent-pent-pent-dhex-dhex-H]- |
| 11 | Spinach | | 1263.5773 [M-pent-H]-; 1087.5323 [M-hexA-pent-H]-; 955.4877 [M-hexA-pent-pent- |
| | | | H]-; 809.3958 [M-hexA-pent-pent-dhex-H]-; 501.3225 [M-hexA-pent-pent-dhex-dhex- |
| | | | hex-H]- |
| 12 | Spinach | | 1099.5310 [M-hexA-pent-H]-; 1057.5170 [M-hexA-pent-Ac-H]-; 501.3190 [M-hexA- |
| | | | pent-pent-pent-dhex-Ac-dhex-H]- |
| 13 | Spinach | | 1129.5430 [M-pent-hexA-H]-; 1087.5220 [M-pent-hexA-Ac-H]-; 501.3220 [M-hexA- |
| | | | pent-pent-hex-dhex-Ac-dhex-H]- |
| 14 | | | |
| 15 | Spinach | VIGS - | 647.3809 [M-hex-H]-; 603.3903 [M-hex-CO2—H]-; 585.3805 [M-hex-CO2—H2O—H]-; |
| | | CYPs | 471.3473 [M-hex-hexA-H]- |
| 16 | Spinach | | 663.3741 [M-hex-H]-; 619.3843 [M-hex-CO2—H]-; 601.3750 [M-hex-CO2—H2O—H]-; |
| | | | 487.3431 [M-hex-hexA-H]- |
| 17 | Spinach | | 809.4302 [M-pent-H]-; 647.3781 [M-pent-hex-H]-; 603.3877 [M-pent-hex-CO2—H]-; |
| | | | 585.3781 [M-pent-hex-CO2—H2O—H]-; 471.3474 [M-pent-hex-hexA-H]- |
| 18 | Spinach | | 809.4344 [M-oxopyruvic and glycolic acid-H]-; 647.3788 [M-hex-oxopyruvic and glycolic acid- |
| | | | H]-; 585.3795 [M-hex-oxopyruvic and glycolic acid-CO2—H2O—H]-; 471.3475 [M-hex- |
| | | | oxopyruvic and glycolic acid-hexA-H]- |
| 19 | Spinach | | 809.4325 [M-oxopyruvic and glycolic acid-H]-; 647.3788 [M-hex-oxopyruvic and glycolic acid- |
| | | | H]-; 585.3802 [M-hex-oxopyruvic and glycolic acid-CO2—H2O—H]-; 471.3477 [M-hex- |
| | | | oxopyruvic and glycolic acid-hexA-H]- |
| 20 | Spinach | | 809.4267 [M-hex-H]-; 417.3479 [M-hex-hex-hexA-H]- |
| 21 | Spinach | | 911.4585 [M-hex-H]-; 647.3829 [M-hex-pent-pent-H]-; 471.3446 [M-hex-pent-pent-hexA-H]- |
| 22 | Spinach | | 955.4252 [M-pent-H]-; 925.4381 [M-hex-H]-; 617.3693 [M-hex-pent-hexA-H]-; 485.3267 |
| | | | [M-hex-pent-pent-hexA-H]- |
| 23 | Spinach | | 1143.5077 [M-pent-]-; 1011.4468 [M-pent-pent-H]-; 677.3535 [M-pent-pent-dhex-Ac-dhex- |
| | | | H]-; 501.3269 [M-pent-pent-dhex-Ac-dhex-hexA-H]- |
| 24 | Spinach | | 793.4354 [M-oxopyruvic and glycolic acid-H]-; 631.3831 [M-oxopyruvic and glycolic acid- |
| | | | hex-H]-; 569.3835 [M-oxopyruvic and glycolic acid-hex-CO2—H2O—H]-; 455.3514 [M- |
| | | | oxopyruvic and glycolic acid-hex-hexA-H]- |
| 25 | Spinach | | 793.4374 [M-oxopyruvic and glycolic acid-H]-; 631.3844 [M-oxopyruvic and glycolic acid-hex- |
| | | | H]-; 569.3845 [M-oxopyruvic and glycolic acid-hex-CO2—H2O—H]-; 455.3517 [M- |
| | | | oxopyruvic and glycolic acid-hex-hexA-H]- |
| 26 | Spinach | | 807.4161 [M-oxopyruvic and glycolic acid-H]-; 645.3607 [M-oxopyruvic and glycolic acid- |
| | | | hex-H]-; 583.3608 [M-oxopyruvic and glycolic acid-hex-CO2—H2O—H]-; 469.3307 |
| | | | [M-oxopyruvic and glycolic acid-hex-hexA-H]- |
| 27 | Spinach | | 1115.5173 [M-pent-H]-; 969.4326 [M-dhex-pent-H]-; 939.4197 [M-hex-dhex-H]-; 823.4352 |
| | | | [M-dhex-pent-dhex-H]-; 485.3267 [M-pent-hex-dhex-dhex-hexA-H]- |
| 28 | Spinach | | 969.4322 [M-dhex-pent-Ac-H]-; 939.4189 [M-hex-dhex-Ac-H]-; 823.4349 [M-dhex-pent- |
| | | | dhex-Ac-H]-; 485.3265 [M-pent-hex-dhex-dhex-hexA-Ac-H]- |
| 29 | | | |
| 30 | Spinach | VIGS − | 955.4204 [M-CO2—H]-; 897.4148 [M-CO2—C2H2O2—H]-; 839.4088 [M- |
| | | GTs+ CSL | CO2—C2H2O2—C2H2O2—H]-; 677.3586 [M-oxopyruvic and glycolic acid-hex-H]-; 663.3751 |
| | | | [M-oxopyruvic and glycolic acid-hexA-H]-; 501.3274 [M-oxopyruvic and glycolic acid-hex-hexA-H]- |
| 31 | Spinach | | 955.4201 [M-CO2—H]-; 897.4130 [M-CO2—C2H2O2—H]-; 839.4070 [M- |
| | | | CO2—C2H2O2—C2H2O2—H]-; 677.3503 [M-oxopyruvic and glycolic acid-hex-H]-; 663.3727 |
| | | | [M-oxopyruvic and glycolic acid-hexA-H]-; 501.3252 [M-oxopyruvic and glycolic acid-hex-hexA-H]- |
| 32 | Spinach | | 939.4240 [M-CO2—H]-; 881.4178 [M-CO2—C2H2O2—H]-; 823.4127 [M- |
| | | | CO2—C2H2O2—C2H2O2—H]-; 677.3565 [M-oxopyruvic and glycolic acid-dhex-H]-; 647.3713 |
| | | | [M-oxopyruvic and glycolic acid-hexA-H]-; 501.3176 [M-oxopyruvic and glycolic acid-hex-hexA-H]- |
| 33 | Spinach | | 647.3743 [M-hexA-pent-H]-; 501.3240 [M-hexA-pent-dhex-H] |
| 34 | Spinach | | 663.3754 [M-hexA-pent-H]-; 501.3202 [M-hexA-pent-hex] |
| 35 | Spinach | | 823.4380 [M-dhex-H]-; 677.3533 [M-dhex-dhex-H]-; 501.3225 [M-dhex-dhex-hexA-H]- |
| 36 | Spinach | | 793.4344 [M-pent-H]-; 631.3828 [M-pent-hex-H]-; 569.3834 [M-pent-hex-CO2—H2O—H]-; |
| | | | 455.3518 [M-pent-hex-hexA-H]- |
| 37 | Spinach | | 647.3774 [M-hex-H]-; 471.3475 [M-hex-hexA-H]- |
| 38 | Spinach | | 645.3638 [M-hex-H]-; 469.3323 [M-hex-hexA-H]- |
| 39 | Spinach | | 661.3593 [M-hex-H]-; 647.3801 [M-hexA-H]-; 617.3719 [M-hex-CO2—H]-; 599.3574 [M-hex- |
| | | | CO2—H2O—H]-; 485.3271 [M-hex-hexA-H]- |
| 40 | Spinach | | 1117.5071 [M-pent-H]-, 985.4347 [M-pent-pent-H]-; 809.4266 [M-pent-hex-dhex-H]-; 501.3194 |
| | | | [M-hexA-hex-pent-pent-dhex-H]- |
| 41 | Spinach | | 985.4608 [M-hex-H]-; 823.3881 [M-hex-hex-H]-; 677.3605 [M-hex-hex-dhex-H]-; 501.3212 |
| | | | [M-hex-hex-dhex-hexA-H]- |
| 42 | Spinach | | 985.4552 [M-pent-H]-; 955.4471 [M-hex-H]-; 677.3557 [M-pent-hex-dhex-H]; 501.3250 [M-pent- |
| | | | hex-dhex-hexA-H]- |
| 43 | Spinach | | 677.3486 [M-hex-H]-, 501.3224 [M-hex-hexA-H]- |
| 44 | Spinach | | 501.3207 [M-hexA-pent-H]-; 325.0773 [hexA + pent-H2O—H]- |
| 45 | Spinach | | 661.3582 [M-hex-H]-; 647.3805 [M-hexA-H]-; 617.3690 [M-hex-CO2—H]-; 599.3580 |
| | | | [M-hex-CO2—H2O—H]-; 485.3252 [M-hex-hexA-H]- |
| 46 | Spinach | | 925.4778 [M-hexA-pent-H]-; 501.3233 [M-hexA-pent-dhex-dhex-pent-H]- |
| 47 | Spinach | | 1143.51 16 [M-pent-H]-; 1101.5002 [M-pent-Ac-H]-; 955.4487 [M-pent-Ac-dhex-H]-; 967.4825 |
| | | | [M-pent-hexA-H]-; 793.4345 [M-pent-hexA-pent-Ac-H]-; 501.3212 [M-dhex-dhex-Ac-pent-pent-hexA-H]- |
| 48 | Spinach | | 663.3739 [M-hex-H]-; 501.3222 [M-hex-hex-H]- |
| 49 | Spinach | | |
| 50 | | | |
| 51 | Medicago | Differential | 825.4302 [M-hex-H]-; 663.3773 [M-hex-hex-H]-; 487.3424 [M0hex-hex-hexA-H]- |
| | sativa | signals form | |
| 52 | Medicago | M sativa | 663.3765 [M-hex-H]-; 487.3432 [M-hex-hexA-H]- |
| | sativa | hairy roots | |
| 53 | Medicago | with silenced | 939.4989 [M-H2O—H]-; 895.5098 [M-CO2—H2O—H]-; 811.4492 [M-dhex-H]-; 767.4695 [M-dhex- |
| | sativa | MsCSL | CO2—H]-; 749.4526 [M-dhex-CO2—H2O—H]-; 631.3887 [M-dhex-hex-H2O—H]-; 613.3731 |
| | | | [M-dhex-hex-H2O—H2O—H]-; 473.3685 [M-hex-dhex-hexA-H]- |
| 54 | Medicago | | 865.4245 [M-CO2—H]-; 823.4179 [M-malonyl-H]-; 805.3989 [M-malonyl-H2O—H]-; 703.3777 [M- |
| | sativa | | hex-CO2—H]-; 643.3525 [M-malonyl-hex-H2O—H]-; 599.3606 [M-malonyl-hex-CO2—H2O—H]-; |
| | | | 485.3273 [M-malonyl-hex-hexA-H]- |
| 55 | Medicago | | 953.4714 [M-H2O—H]-; 909.4915 [M-CO2—H2O—H]-; 825.4289 [M-dhex-H]-; 763.4302 [M-dhex- |
| | sativa | | CO2—H2O—H]-; 645.3669 [M-dhex-hex-H2O—H]-; 601.3768 [M-dhex-hex-H2O—H2O—CO2—H]-; |
| | | | 555.3728 [M-dhex-hex-108-H]-; 487.3447 [M-dhex-hex-hexA-H]- |
| 56 | Medicago | | 1099.5315 [M-H2O—H]-; 955.4952 [M-hex-H]-; 893.5005 [M-hex-CO2—H2O—H]-; 791.4280 |
| | sativa | | [M-hex-dhex-H2O—H]-; 747.4368 [M-hex-dhex-CO2—H2O—H]-; 729.4260 [M-hex-dhex- |
| | | | H2O—H2O—C2O—H]-; 629.3726 [M-hex-dhex-hex-H]-; 585.3829 [M-hex-dhex-hex-CO2—H]-; |
| | | | 539.3792 [M-hex-dhex-hex-108-H]-; 471.3504 [M-hex-dhex-hex-hexA-H]- |
| 57 | Medicago | | 661.3595 [M-hex-H]-; 617.3698 [M-hex-CO2—H]-; 599.3587 [M-hex-CO2—H2O—H]-; 485.3268 |
| | sativa | | [M-hex-hexA-H]- |
| 58 | Medicago | | 1085.5536 [M-H2O—H]-; 1041.5638 [M-H2O—C2O—H]-; 957.5061 [M-dhex-H]-; 895.5071 [M-dhex- |
| | sativa | | H2O—C2O—H]-; 795.4548 [M-dhex-hex-H]-; 777.4427 [M-dhex-hex-H2O—H]-; 759.4297 [M-dhex-hex- |
| | | | H2O—H2O—H]-; 457.3681 [M-hex-hex-dhex-hexA-H]- |
| 59 | | | |
| 60 | Beta | Differential | 997.5342 [M-C3H4O5—H]-; 955.5225 [M-Act-H]-; 835.4744 [M-C3H4O5—Hex-H]-; 793.4632 [M-Act- |
| | vulgaris | signals from | hex-H]-; 731.4594 [M-Act-hex-CO2—H2O—H]-; 631.4028 [M-Act-hex-hex-H]-; 613.3919 [M-Act- |
| | | B. vulgaris | hex-hex-H2O—H]-; 455.3650 [M-Act-hex-hex-hexA-H]- |
| 61 | Beta | with silenced | 955.5191 [M-pent-H]-; 925.5099 [M-hex-H]-; 793.4655 [M-hex-pent-H]-; 731.4594 [M-hex-CO2-pent- |
| | vulgaris | BvCSL | H2O—H]-; 613.3902 [M-hex-pent-hex-H2O—H]-; 569.4006 [M-hex-pent-hex-H2O—C2O—H]-; |
| | | | 551.3894 [M-hex-pent-hex-H2O—C2O—H2O—H]-; 455.3650 [M-hex-pent-hex-hexA-H]- |
| 62 | Beta | | 967.5213 [M-C3H4O5—H]-; 925.5092 [M-Act-H]-; 805.4618[M-C3H4O5-hex-H]-; 763.4507 [M-hex- |
| | vulgaris | | Act-H]-; 743.4590 [M-C3H4O5-hex-H2O—C2O—H]-; 593.3999 [M-C3H4O5-hex-H2O—CO2-pent-H2O—H]-; |
| | | | 455.3639 [M-Act-hex-pent-hexA-H]- |
| 63 | Beta | | 763.4516 [M-hex-H]-; 701.4487 [M-hex-CO2—H2O—H]-; 631.4028 [M-hex-pent-H]-; 569.4016 [M-hex- |
| | vulgaris | | CO2—H2O-pent-H]-; 551.3904 [M-hex-CO2—H2O-pent-H2O—H]-; 455.3654 [M-hex-pent-hexA-H]- |
| 64 | Beta | | 835.4753 [M-C3H4O5—H]-; 793.4630 [M-Act-H]-; 673.4167 [M-C3H4O5-hex-H]-; 631.4045 [M-Act-hex- |
| | vulgaris | | H]-; 569.4015 [M-Act-hex--CO2—H2O—H]-; 455.3655 [M-hex-hexA-Act-H]- |
| 65 | | | |
| 66 | Expression in | genes from | 501.3206 [M-GlcA-H]-; 483.3096 [M-GlcA-H2O—H]-; 439.3184 [M-GlcA-H2O—C2O—H]-; 193.0346 |
| | N. benthamiana | spinach | [GlcA-H]-; 175.0238 [GlcA-H2O—H]- |
| 67 | Expression in | | 647.3776 [M-hexA-H]-; 501.3237 [M-hexA-dhex-H]- |
| | N. benthamiana | | |
| 68 | Expression in | | 793.4233 [M-hexA-H]-; 501.3219 [M-hexA-dhex-dhex-H]-; 439.3192 [M-hexA-dhex-dhex-CO2—H2O—H]- |
| | N. benthamiana | | |
| 69 | Expression in | | 955.4902 [M-hexA-H]-; 677.3536 [M-hex-dhex-dhex-H]-; 501.3195 [M-hexA-hex-dhex-dhex-H]- |
| | N. benthamiana | | |
| 70 | Expression in | | 955.4648 [M-hexA-pent-H]-; 793.4109 [M-hexA-pent-hex-H]-; 501.3018 [M-hexA-pent-hex-dhex-dhex-H]-; |
| | N. benthamiana | | 483.3283 [M-hexA-pent-hex-dhex-dhex-H2O—H]- |
| 71 | Expression in | | 997.4995 [M-hexA-pent-H]-; 955.4883 [M-hexA-pent-Ac-H]-; 501.3227 [M-hexA-pent-hex-dhex-dhex-Ac-H]-; |
| | N. benthamiana | | 439.3179 [M-hexA-pent-hex-dhex-dhex-Ac-CO2—H2O—H]- |
| 72 | | | |
| 73 | Expression in | genes from | 583.3629 [M-CO2—H2O—H]-; 569.3469 [M-C2O3H4—H; ^{0, 4}X]-; 523.3415 [M-C2O3H6—H2O—H; |
| | N. benthamiana | G. uralensis | ^{0, 3}X-H2O]-; 469.3315 [M-GlcA-H]-; 425.3419 [M-GlcA-CO2—H]- |
| 74 | Expression in | | 583.3635 [M-CO2—H2O—H]-; 569.3470 [M-C2O3H4—H; 0^{0, 4}X]-; 523.3423 [M-C3O4H6—H2O—H; |
| | N. benthamiana | | ^{0, 3}X-H2O]-; 469.3318 [M-GlcA-H]-; 425.3419 [M-GlcA-CO2—H]- |
| 75 | | | |
| 76 | Expression in | | 583.3631 [M-CO2—H2O—H]-; 569.3468 [M-C2O3H4—H; ^{0, 4}X]-; 523.3418 [M-C3O4H6—H2O—H; |
| | N. benthamiana | | ^{0, 3}X-H2O]-; 469.3317 [M-GlcA-H]-; 425.3422 [M-GlcA-CO2—H]- |
| 77 | Expression in | | 583.3633 [M-CO2—H2O—H]-; 569.3470 [M-C2O3H4—H; ^{0, 4}X]-; 523.3421 [M-C3O4H6—H2O—H; |
| | N. benthamiana | | ^{0, 3}X-H2O]-; 469.3320 [M-GlcA-H]-; 425.3415 [M-GlcA-CO2—H]- |
| 78 | | | |
| 79 | Expression in | | 645.3636 [M-GlcA-H]-; 469.3303 [M-GlcA-GlcA-H]-; 351.0574 [GlcA + GlcA-H]-; 193.0357 [GlcA-H]-; |
| | N. benthamiana | | 175.0258 [GlcA-H2O—H]- |
| 80 | Expression in | | 645.3615 [M-GlcA-H]-; 469.3323 [M-GlcA-GlcA-H]-; 351.0577 [GlcA + GlcA-H]-; 193.0354 [GlcA-H]-; |
| | N. benthamiana | | 175.0250 [GlcA-H2O—H]- |
| 81 | | | |
| 82 | Expression in | | 501.3212 [M-GlcA-H]-; 483.3084 [M-GlcA-H2O—H]-; 439.3176 [M-GlcA-H2O—C2O—H]-; 193.0334 |
| | N. benthamiana | | [GlcA-H]-; 175.0342 [GlcA-H2O—H]- |

Section B

| | Q | R | S | T |
|---|---|---|---|---|
| 1 | MS/MS ES(−) CE (eV) | UV/Vis | Detected in other species | References |
| 2 | ramp 15-50 eV | — | — | — |
| 3 | ramp 15-50 eV | — | — | — |
| 4 | ramp 15-50 eV | — | — | — |
| 5 | 70 | — | — | — |
| 6 | 70 | — | — | — |
| 7 | ramp 15-50 eV | — | — | — |
| 8 | 70 | — | — | — |
| 9 | 70 | — | — | — |
| 10 | 70 | — | — | — |
| 11 | 70 | — | — | — |
| 12 | 70 | — | — | — |
| 13 | 70 | — | — | — |
| 14 | | | | |
| 15 | 60 | — | — | — |
| 16 | ramp 15-50 eV | — | — | — |
| 17 | ramp 15-50 eV | — | — | — |
| 18 | 60 | — | — | — |
| 19 | ramp 15-50 eV | — | — | — |
| 20 | ramp 15-50 eV | — | — | — |
| 21 | ramp 15-50 eV | — | — | — |
| 22 | ramp 15-50 eV | — | — | — |
| 23 | ramp 15-50 eV | — | — | — |
| 24 | 60 | — | Beta vulgaris/Basella rubra | (54) |
| 25 | ramp 15-50 eV | — | Beta vulgaris/Basella rubra | (54) |
| 26 | 60 | — | Basella rubra | (55) |
| 27 | ramp 15-50 eV | — | — | — |
| 28 | ramp 15-50 eV | — | — | — |
| 29 | | | | |
| 30 | ramp 15-50 eV | — | — | — |
| 31 | ramp 15-50 eV | — | — | — |
| 32 | ramp 15-50 eV | — | — | — |
| 33 | 50 | — | Beta vulgaris | (56) |
| 34 | 50 | — | — | — |
| 35 | ramp 15-50 eV | — | — | — |
| 36 | ramp 15-50 eV | — | — | — |
| 37 | ramp 15-50 eV | — | — | — |
| 38 | ramp 15-50 eV | — | — | — |
| 39 | ramp 15-50 eV | — | — | — |
| 40 | ramp 15-50 eV | — | — | — |
| 41 | ramp 15-50 eV | — | — | — |
| 42 | ramp 15-50 eV | — | — | — |
| 43 | ramp 15-50 eV | — | — | — |
| 44 | ramp 15-50 eV | — | — | — |
| 45 | 50 | — | — | — |
| 46 | 70 | — | — | — |
| 47 | ramp 15-50 eV | — | — | — |
| 48 | ramp 15-50 eV | — | — | — |
| 49 | | — | — | — |
| 50 | | | | |
| 51 | 45 | — | Medicago truncatula | (Main text reference 14) |
| 52 | 45 | — | Medicago truncatula | (Main text reference 14) |
| 53 | 45 | — | Medicago truncatula | (Main text reference 14) |
| 54 | 45 | — | Medicago truncatula | (Main text reference 14) |
| 55 | 60 | — | Medicago truncatula | (Main text reference 14) |
| 56 | 60 | — | Medicago truncatula | (Main text reference 14) |
| 57 | 45 | — | Medicago truncatula | (Main text reference 14) |
| 58 | 45 | — | Medicago truncatula | (Main text reference 14) |
| 59 | | | | |
| 60 | 60 | — | Beta vulgaris | (57) |
| 61 | 60 | — | Beta vulgaris | (57) |
| 62 | 60 | — | Beta vulgaris | (57) |
| 63 | 60 | — | Beta vulgaris | (57) |
| 64 | 60 | — | Beta vulgaris | (57) |
| 65 | | | | |
| 66 | 45 | — | — | — |
| 67 | 45 | — | — | — |
| 68 | 50 | — | — | — |
| 69 | 55 | — | — | — |
| 70 | 70 | — | — | — |
| 71 | 70 | — | — | — |
| 72 | | | | |
| 73 | 55 | — | Glycyrrhiza uralensis | (58) |
| 74 | 55 | — | Glycyrrhiza uralensis | (58) |
| 75 | | | | |
| 76 | 55 | — | Glycyrrhiza uralensis | (58) |
| 77 | 55 | — | Glycyrrhiza uralensis | (58) |
| 78 | | | | |
| 79 | 65 | — | Glycyrrhiza uralensis | (58) |
| 80 | 65 | — | Glycyrrhiza uralensis | (58) |
| 81 | | | | |
| 82 | 45 | — | — | — |

TABLE 13
Summary of Saponin Composition in Spinach

| | | Molecular | m/z measured | m/z calculated | Δm/z |
|---|---|---|---|---|---|
| No. | Name | Formula | [M-H] | [M-H] | ppm |
| 1 | Yossoside XI | C64H100O33 | 1395.6068 | 1395.6069 | −0.1 |
| 2 | Yossoside VI | C47H88O30 | 1131.5261 | 1131.5282 | −1.9 |
| 3 | Yossoside IV | C52H96O34 | 1263.5713 | 1263.5705 | 0.6 |
| 4 | Yossoside VIII | C63H98O32 | 1365.5981 | 1365.5963 | 1.3 |
| 5 | Yossoside XII | C58H90O28 | 1233.5554 | 1233.5540 | 1.1 |
| 6 | Yossoside Va | C61H94O30 | 1306.5773 | 1305.5752 | 1.6 |
| 7 | Yossoside X | C66H102O34 | 1437.6160 | 1437.6161 | 0.6 |
| 8 | Yossoside VII | C60H92O29 | 1275.5675 | 1275.5646 | 2.3 |
| 9 | Yossoside V | C61H94O30 | 1305.5781 | 1305.5752 | 2.2 |
| 10 | Yossoside IX | C65H100O33 | 1407.6107 | 1407.6069 | 2.7 |
| 11 | Yossoside VIIa | C60H92O29 | 1275.5640 | 1275.5646 | −0.5 |

Mass spectrometry fragmentation analysis and a series of ID and 2D-NMR experiments established the structure of the most abundant spinach saponin (termed here Yossoside V). It comprises medicagenic acid (the aglycone) with glucuronic acid and xylose attached to the hydroxyl at the C-3 position and acetyl-fucose, rhamnose and glucose linked to the carboxyl at C-28 (FIGS. 20A-20C, FIG. 21).

Summary: Eleven triterpenoid saponins were identified in spinach, wherein Yossiside V was the most abundant.

### Example 16: Identification of Saponin Biosynthesis Genes Using Spinach Transcriptome Data

Objective: To identify triterpenoid aglycone biosynthesis genes in the triterpenoid saponin biosynthetic pathway.

Methods: See Materials and Methods above.

Results: Transcriptome data was generated from five spinach tissues exhibiting varying content of saponins, and gene candidates selected based on their differential expression between samples with high and low saponin content, as well as their homology to known triterpenoid aglycone biosynthesis genes (E. Biazzi et al., CYP72A67 Catalyzes a Key Oxidative Step in Medicago truncatula Hemolytic Saponin Biosynthesis. Molecular Plant. 8, 1493-1506 (2015)).

Inspecting the genomic location of three of the candidates, including: saponin β-amyrin synthase (SobAS; termed SOAP1; Sp_107620_kpnh; SEQ ID NO: 45 [gene sequence] SEQ ID NO: 48 [polypeptide sequence]), cytochrome P450 CYP716A268 (SOAP2; Sp_107660_kiqg; SEQ ID NO: 46 [gene sequence], SEQ ID NO: 49 [polypeptide sequence]), and CYP716A268v2 (SOAP2-like; Sp_107670_ptqx; SEQ ID NO: 47 [gene sequence] SEQ ID NO: 6 [polypeptide sequence]) revealed their close physical genomic location in the form of a metabolic gene cluster (FIGS. 22A and 22B).

Virus Induced Gene Silencing (VIGS) assays of SOAP1 and SOAP2 in spinach resulted in reduced saponin content (FIGS. 23A-23D), while their over-expression in N. benthamiana resulted the production of β-amyrin and oleanolic acid (FIGS. 24A and 24B).

Summary: These results provided strong evidence for the involvement of these genes in spinach saponin biosynthesis.

### Example 17: Co-Expression Analysis for Identification of Additional Saponin Biosynthesis Genes & Characterization of Enzymes Encoded

Objective: To identify additional triterpenoid aglycone biosynthesis genes in the triterpenoid saponin biosynthetic pathway.

Methods: See Materials and Methods above.

Results: SOAP1, SOAP2 and SOAP2-like nucleotides (nucleotide sequences SEQ ID NOs: 45-47 were expressed were subsequently used as baits in co-expression analysis (r >0.9; Pearson correlation coefficient—PCC) and an additional five genes encoding p450 cytochromes, eight genes encoding glycosyltransferases, and five genes encoding acyltransferases were identified for functional characterization using VIGS (FIGS. 20A and 20B; Table 14). [SOAP1, Soap2, and SOAP2-like nucleotide encode polypeptide sequences: SEQ ID NO: 48, SEQ ID NO: 49, and SEQ ID NO: 50, respectively) These experiments revealed that CYP72A655 (SOAP3; Sp_085340_meek; SEQ ID NO: 51 [nucleotide], SEQ ID NO: 52 [polypeptide]) and CYP72A654 (SOAP4; Sp_040350_wdny; SEQ ID NO: 53 [nucleotide], SEQ ID NO: 54 [polypeptide]) participate in the biosynthesis of the Yossoside aglycone (FIG. 25). In Table 14 below, terms used include: coexpressed gene—gene with coexpression correlation coefficient greater than 0.9; annotation—gene functional annotation based on blast analysis against Arabidopsis proteome (TAIR10); and aa seq—amino acid sequence of coexpressed gene Table 14: Coexpression analysis of spinach genes with three baits SOAP1, SOAP2 and SOAP2-like.

Lengthy table referenced here
US20240315192A1-20240926-T00001
Please refer to the end of the specification for access instructions.

Down regulation of SOAP3 and SOAP4 expression led to reduced accumulation of medicagenic acid derived saponins and caused accumulation of glycosylated intermediates (augustic acid, bayogenin and hederagenin; (FIG. 25, FIGS. 26A-26L, FIG. 27, FIGS. 28A-28C).

The structure of accrued pathway intermediates demonstrated that SOAP3 (SEQ ID NO: 52) is a C-2 hydroxylase and SOAP4 (SEQ ID NO: 54) is a C-23 oxidase (FIG. 20A). CYP2 (CYP72A656; Sp_148230_dgra) (SEQ ID NO: 72 [gene] and SEQ ID NO: 73 [polypeptide]), which was also identified, displays high homology to SOAP4 (almost 92% at the amino acid level), likely exhibits the same activity in the saponin biosynthesis pathway. Additionally, reduced saponin content was observed in plants with silenced: SOAP6 (UGT74BB2; Sp_170930_hjgq; SEQ ID NO: 55 [gene] and SEQ ID NO: 56 [polypeptide]), SOAP7 (UGT79K1; Sp_020820_yeau; SEQ ID NO: 57 [gene] and SEQ ID NO: 58 [polypeptide]), SOAP8 (UGT79L2; Sp_113700_suxh SEQ ID NO: 59 [gene] and SEQ ID NO: 60 [polypeptide]) and SOAP9 (UGT73BS1; Sp_170320_dmqi SEQ ID NO: 61 [gene] and SEQ ID NO: 62 [polypeptide]) glycosyltransferases (FIG. 29).

The same plants accumulated pathway intermediates lacking certain sugars: deoxyhexoses (SOAP6 (SEQ ID NO: 56) and SOAP7 (SEQ ID NO: 58)), hexose (SOAP8 (SEQ ID NO: 60)) and pentose (SOAP9 (SEQ ID NO: 62)) (FIGS. 30A-30C). Moreover, VIGS of one of the five co-expressed acyltransferases (AT) (i.e., SOAP10; Sp_125800_kzws; SEQ ID NO: 63 [gene], SEQ ID NO: 64 [polypeptide]) resulted in an increased ratio of desacetyl to acetylated saponins (from 20 to 80%) (FIGS. 31A-31C). The acetyltransferase activity of recombinant SOAP10 expressed in E. coli was subsequently confirmed in vitro, as it acetylated Yossoside IV and other Yossosides (e.g., Yossoside VI and Yossoside XII) with two deoxyhexoses attached at the C-28 position (FIGS. 14A-14C).

### Example 18: Validation of SOAP Genes

Objective: To validate the function of all nine genes [i.e., β-amyrin synthase (bAS; SOAP1; SEQ ID NO: 45); cytochrome P450 (CYP450s; SOAP2, 3, 4; SEQ ID NO: 46, 51, 53, respectively); UDP-glycosyltransferases (UGTs; SOAP 6, 7, 8, 9; SEQ ID NO: 55, 57, 59, 61 respectively) and acyltransferase (AT; SOAP10; SEQ ID NO: 63)]

Methods: See Materials and Methods above.

Results: The nine genes of interest (SOAP 1-4 and 6-10) were transiently expressed in N. benthamiana leaves. Expression of the first four biosynthetic genes (SOAP1 to SOAP4) resulted in the formation of medicagenic acid (FIG. 20A). Curiously, co-expression of all nine genes did not lead to production of any saponin that had previously been detected in spinach leaves. Nevertheless, the accumulation of medicagenic acid and glycosylated derivatives [MA+hex; MA+2×hex, MA+3×hex) that are normally not present in spinach leaves were detected (FIGS. 32A-32D).

Summary: While reconstitution of medicagenic acid was achieved, expression of the nine genes SOAP1-4 and 6-10 did not result in the production of any saponins.

### Example 19: Analysis of Cellulose Synthase Like G Spinach Homolog as a Component of the Saponin Biosynthetic Pathway

Objective: To identify the missing gene or genes necessary for reconstitution of the biosynthetic pathway of saponins.

Methods: See Materials and Methods above.

Results: At this point, it was realized that at least one enzyme was absent in order to reconstitute the saponin biosynthetic pathway. In an effort to identify the missing enzyme or enzymes the list of genes obtained by co-expression analysis was revisited. The only gene co-expressed with all baits in the co-expression set that appeared related to sugar metabolism was a Cellulose Synthase Like G spinach homolog (SoCSLG) (T. Richmond, Higher plant cellulose synthases, Genome Biology. 1, 3001.1-3001.6 (2000).). Although its contribution to saponin biosynthesis seemed unlikely based on this functional annotation, its function within this pathway was studied.

Notably, SoCSLG silencing resulted in high accumulation of medicagenic acid in spinach leaves (FIGS. 33A-33F; FIGS. 34A and 34B) suggesting its role in triterpenoid saponin biosynthesis. Next, SoCSLG (SEQ ID NOS: 65 OR 93) was transiently expressed together with SOAP1-4 (SEQ ID NO: 45, SEQ ID NO: 46, SEQ ID NO: 51, SEQ ID NO: 53, respectively), in N. benthamiana leaves. The combined activity of all five enzymes resulted in the formation of medicagenic acid 3-O-glucuronide (MA-3-GlcA); the first glycosylated intermediate in the spinach triterpenoid saponin biosynthetic pathway (FIGS. 20A-20C and FIGS. 32A-32F; FIGS. 35A-35C).

Furthermore, combinatorial expression of CSLG (SOAP5, Sp_076690_ejcm (SEQ ID NOS: 65 or 93 [gene], SEQ ID NO: 66 [polypeptide]) with bAS (SOAP1; SEQ ID NO: 45) and CYP450s (SOAP2 [SEQ ID NO: 46], SOAP3 [SEQ ID NO: 51] and SOAP4 [SEQ ID NO: 53]) demonstrated that apart from medicagenic acid, SOAP5 [SEQ ID NO: 66] could glucuronidate other triterpenoid aglycones including oleanolic acid, augustic acid, hederagenin, gypsogenin and gypsogenic acid, though they were never detected in significant amounts in spinach (FIGS. 36A-36F). SOAP5 (SoCSLG) was identified as the spinach saponin glucuronic acid transferase. Conversely, inhibition of expression of SOAP5 (SEQ ID NOS: 65 or 93) could in some embodiments, results in a decrease or inhibition of glucuronidating triterpenoid aglycones including but not limited to medicagenic acid, oleanolic acid, augustic acid, hederagenin, gypsogenin, and gypsogenic acid.

To examine if SOAP5 was indeed a missing component in the spinach saponin pathway a combination of all ten SOAP genes (See Table 16) were transiently expressed in N. benthamiana leaves. Metabolite analysis showed the presence of the most abundant spinach saponin Yossoside V signifying the identification of all required steps and enzymes in the native pathway (FIGS. 33A-33F).

Summary: Enzymes for all required steps in the native pathway of spinach saponin Yossoside V (Compound 11) have been identified and demonstrated to be functionally active in a heterologous plant system.

### Example 20: Characteristics of the Triterpenoid Biosynthetic Pathway

Objective: To elucidate further details of the spinach saponin metabolic pathway.

Methods: See Materials and Methods above.

Results: The identification of SOAP5 (SEQ ID NO: 66) as the spinach saponin glucuronic acid transferase enabled deciphering the pathway further. It was discovered that SOAP6 (SEQ ID NO: 56) works directly on MA-3-GlcA linking a fucose to the carboxyl group at position C-28 of the aglycone, giving rise to Yossoside I (Compound 7). In fact, this makes SOAP6 the first characterized fucosyltransferase involved in triterpenoid metabolism (T. Louveau et al., Analysis of two new arabinosyltransferases belonging to the carbohydrate-active enzyme (CAZY) glycosyl transferase family 1 provides insights into disease resistance and sugar donor specificity. The Pant Cell. 30, 3038-3057 (2018).) (FIGS. 37A-37E, FIGS. 38A-38E).

Expression of SOAP7 (SEQ ID NO: 57) generated the Yossoside II product only when expressed together with the other six SOAP1-6 genes (See Table 16). Based on the LC-MS/MS analysis, SOAP7 (SEQ ID NO: 58) is a glycosyltransferase transferring rhamnose onto the fucose of Yossoside I (FIGS. 37A-37E, FIGS. 38A-38E). Yossoside II is further converted into Yossoside III by SOAP8 (SEQ ID NO: 60), a glucosyltransferase that accepts as a substrate only an aglycone having a fucose and rhamnose attached to the C-28 position (FIG. 20A; FIGS. 37A-37E, FIGS. 38A-38E). Expression of SOAP9 (SEQ ID NO: 61 [gene], SEQ ID NO: 62 [polypeptide]) showed that it is a xylosyltransferase attaching a pentose to glucuronic acid linked to the MA at C-3. In case of SOAP9, the sugar moieties decorating the triterpenoid backbone at C-28 are not crucial for its activity (FIGS. 39A-39C). The in planta and in vitro studies showed that SOAP10 takes part in last step of saponin biosynthesis in spinach transferring acetyl group to the fucose moiety (FIGS. 31A-31F). The nucleotide and amino acid sequences of the acetyl transferases used for comparison are presented below in Table 15.

TABLE 15
Nucleotide and Amino Acid Sequences of Acetyl Transferase Enzymes

| | SEQ | |
|---|---|---|
| | ID | |
| NAME | NO: | AA SEQ/NT SEQ |
| sp_074630_ | 109 | ATGGCCAAATCTGAGCAAGAAACAATGGCCAAATCTGAGCAAGAAACATCCAT |
| ygho_ | | CAAACTTGTTTCAGAATGCTTTGTAAAACCAAAATATGAGATTAAATCCGCTAA |
| Spo04549_ | | GCAACCTTACCACTTAGGTCCCATGGATCTAGTTATGTTAACTATCGATCCTAT |
| AT1 | | ACAAAAAGGTCTTGTCTTTACAATAAAGAATTCCCCACTCTTTTTGTCATCCGA |
| gene | | ATCCCATGATAATATTGAAATTATTCGAACCAAAGTTGTGTCACGTTTATTAGA |
| | | AAAGCTTAAACACTCACTTTCTATAGCTCTAGTCCACTTCTACCCGTTAGCAGG |
| | | TCGTTTCACTACACAAAAACAACCCGAGCATAACACGAGCTTGGTCTTTATTGA |
| | | TTGCAACAAAGGTCCCGGAGCGCGGTTCATCCACGCTACTTCCCTTGACTTTAC |
| | | TATCTCCGATATACTTTCACCGGTTGATGTTTCCATCGTTCATTCTTTCTTTGATC |
| | | TCGGTGAAAAGCATGTAAACTACGATTGTCATACTAAGGCGTTGCTATCGATCC |
| | | AGGTAACAGAACTTTTAGATGGGGTGTTTATTGGGTTTAGCATGAGTCATAGTG |
| | | TGGTTGATGGTACCTCTTTTATTCATTTTGTCAATACCTTGTCTGAAATTTTTAA |
| | | ATCTGATGATTTTACCACTATTTCACGTGCCCCAATACTTAATTATAGGCCTTGT |
| | | GATATTCCGATCCTTAAATTTCCGTTTCTTGATGTGGAGGGGTTTATATGTCGTG |
| | | CGTATAACCCTGGGCCGTTAAGGGAAAGAATCTTCCACTTTTCACTAAATTCGA |
| | | TGCTGAGACTCAAGGCCATGGCTAACCAAGAATGTGGTACCCAAAATGTTTTA |
| | | TCATCTTTCCAAGCTTTGACTGCGGTTGTATGGAGGTCCATCACCCGAGTTCGG |
| | | AACTTACCAAAGGATGAGCAAACCACGTGTTTTATGGCTATGGGTTCTCGAACT |
| | | AGGCTCAACCCGCCTCTTTCGGATGACTATTTTGGGAATTTTATGATTAGTACC |
| | | AAATTTGCTTGCAAGGCAGAGGAATTATTGGGTAACAGTTTAGGTTGGGTAGC |
| | | AATGAATTTACGTAAAATCATTATGTCCACTGACGAGAAATCGATACTTGCTAC |
| | | GTACAAAGCATTGGCTGATTCCCCAATAGTGATTCCGCGTGAAACGATCCCCG |
| | | GTCCTCATGGGATGACCAGAGTAATAATTGGAGGATCTTCAAGGTTCGATATG |
| | | TATGGGCCTGAATTTGGATTGGGTCGAGCTTTGGCCGCTCGCATGGGTTATGGG |
| | | AATAAGGATGATGGGAAAATAACAGCAAATCCTGGGTGTGAAGGAGGTGGAA |
| | | GTGTTGATTTGGAAATTTGCCTTAGGCCTCATATTATGGCCTCTCTTGAAGTTGA |
| | | TCAAGAGTTTATGGGTTTTGTGTCCTAG |
| sp_123780_ | 110 | ATGACTCCAAATCTGCAAATAGTAACCAACGGAGGCAAACCGGAAAATGATG |
| pgiy_ | | AAGCAGAACCCGTATCACCTACCGGACAATACTTCAACAGCAAAGTGTTGTCT |
| Spo21561_ | | GTTTGTGTCCTTGCCATTCTAGAAATTGATGTTCCTATAGATGACTCGTGTGTAA |
| AT2 | | TTCCACAACTCCGTGATGTCTTCCTACCCATGAACCCCAGATTTTCATCTATCAT |
| gene | | GATATCTGACAATAAAGATGTAAAACAATGGAAAAGAGTGGAAGTGAACCTTC |
| | | AAGATCATGTTGTCGTCCCTAGCGTCCCAGATGGCTTATCGGTTGAATCATACG |
| | | ACAAGTACTTTGATGAATATCTGACAAAAATAACAGTGGATCCATTACCACAG |
| | | GATAGGCCTTTATGGGAACTTCATGTTATAAAATACCCAACAAGCAAAGCAGC |
| | | GGGTCATTTCATCTGGAAGCTTCACCATGCACTTGGTGACGGCTACACTCTAAT |
| | | GGGAGTACTTCTGTCCGGCGTGAACAGAGCAGATGATCCTTCCCTTCCGTTAAC |
| | | TTTCCCTTCAACACGATCAAGCTCACTAGTTACAAACAACAAGATGAATATTAT |
| | | CAGCTGGGTGCCAAGAACTTTTTCAGCAATCTACAACGGTGTTTATAATTTTGG |
| | | ATGGAGTTTTCTAAAAAGCACTTGCAAGGCAGATGATAAGACACCTATCAGAT |
| | | CCGGAAATGAAGGTCTGGGTTTCCACCCAATGAAGATCTCGACAATAGAACTA |
| | | TCCCTAGACCAAATCAAATTTATCAAAACAAAACTCGGCGCAACGGTAAATGA |
| | | CATTCTTGCAGGCATAATTTTCCTCGGGGTTCGAAAATACATGCAAGCAACTGA |
| | | TACAGAATCTGGAAACTCAGAATCAACGGCATTGGTGCTGTTTAACACTAGGA |
| | | ACATTGGAGGTTATATGACCGCTGAGCAAATGAAGAAAGCACAAATGAAAAT |
| | | ATGGGGGAACCAATTTGCATTTTTGCATATAGCAATACCTCAATTAATCAATGA |
| | | CAAATGCTCGAACCCCCTTGACTATGTCTATGAAGCACGAAAACAGATCTCTA |
| | | GGTTCAAAAGCTCACCATCAGTCTATCTAACAGCTCAGTGCCTAGAGCTGCTAG |
| | | GAAATGCAAAGGACCTGAGGCAGCAGCTGAATTTATCCAGAGTACAACGAATA |
| | | AAGCAAGCATATTAA |
| sp_149180_ | 111 | ATGTTAGAGCTAGCAGAAGACGAGGTGAAGAACTTCTTCAAGGTATGGGCAAT |
| nwmy_ | | AGTTTTTGCATCTTTAAGCTATTGTTATTACATAGGCAAGCTAATTAATCCAAA |
| Sp015788_ | | AGGTTATACAAGATTAGTAGCAATAATCCCAATTATTACTCTCTTTTTAGCACT |
| AT3 | | TCCTTTAAATCTCACATCTTTTCATCTTGGTGGTATGACTTGTTTCTTTATTGCTT |
| gene | | GGCTTGCTAATTTCAAACTCTTGCTTTTTGCTTTTGATAAGGGCCCACTTTGCGC |
| | | TAATTCTTCAATCTCATTCGCCAAATTTCTTGCACTTTCTTGCTTACCCATCAAA |
| | | ATCCAACACCCACCTCATAAAAAGTCATTAAAATCACACCCATCTATTTATAAT |
| | | TACATCATTAAAGGGATACTTTTATGTCTAATAATTAAAATCTATGATTATGGT |
| | | GATTACATTCATCCAAAAATCATATGGCTAATCTTTTTCTTCCACTCCTATTTTA |
| | | CCATAGAGTTAGTCTTTGCATTCCTAGCAACATCGACTAATATTTTGTTAGGGC |
| | | TCGAACTGGAGCCACAGTTCAATGAACCCTTAATATCAACCTCATTGCAAGACT |
| | | TTTGGGGTAAGAGATGGAATATCATGGTGACAAGGATACTTAGGCCTACGGTG |
| | | TACCTTCCCACACTAGAGTACTCCACTAAGGTCGTTGGACGCACGTGGGCCAC |
| | | ACTTCCGGCGGTGATGTCCACGTTCTTTGTGTCAGCCATTATGCACGAGCTCAT |
| | | CTTCTACTACTTGGGGCGCAACTGGCCCACATTCGAGGTGACGTGGTTCTTTCT |
| | | CCTGCATGGATTATGTCTTTGTGTTGAGATTGTCGCTAAGAAGTTAGTTGGTGG |
| | | GAAATGGAGGATCCCACGGTGGATTTCCGGCCCTGCCACGGTGTTGTTTGTGGT |
| | | GGGTACTGGGTTTTGGCTGTTCTTGCCGCCGTTGTTGAAGGCTGGGTTGGATAC |
| | | TAGACCGTTTCAAGAGTTTGCGGCCGTTGCCAAGTTTGTAAGGAGTTTGAAGGC |
| | | AGCTCTCACATTTTG |
| sp_198340_ | 112 | ATGGCTCCTCCTTCTTCTTCTTCAACTACGGGTTCTGGTAATGGTTCTAGTTTTG |
| focw_ | | CAGTCAATATAATGGCGTCGTTCTACATTTCCCCACAACAACCTTCCACCACAA |
| Spo13090_ | | ATTCACATTCTATCCCTCTCACTTTCTTTGACATTCCTTGGCTTCAATATCCTCC |
| AT4 | | GCTCCAACCTCTCTTCTTCTTTCAACTTCCATCTACACCCCAATCTTCTTCTTCTT |
| gene | | CTTCTTCTTCTTCTTCTTCTTCTTTCGACCACAACTTGTACTTGGAGTTTAGCTCC |
| | | ACCATCCTCCCTAGGCTCAAACACTCCCTCGCTTCTGCCTTGCAATATTACTTTC |
| | | CCTTTTCTGGAAAACTCACCACTACTACCCATACTATCCCGAATAACCTAGTTT |
| | | TCTCGACAGACTCATCAGATTCTGTTGAGTTGACTGTTTCTCTGTGTGATGCTGA |
| | | TTTTAATGGTCTATGCAGCTTTCTACCCAGGTCTACTCATCTCTTCCAACAATTG |
| | | GTTCCCTCCTTGCCAAATATTGAATCCTCCAACCTCACTACATTCCCTGCACCTT |
| | | TATTAGCTATTCAGATCACATTCTTTCCCACCTCTTCTCCTGGTTTCTCTATTGG |
| | | CTTTGCTTCTCATCCTGTGCTTTCTGATCAGAGGACCTTCAGTAACTTCCTTTAC |
| | | TCTTGGGCCTCTTTCTCCAAGTTTGATAATCTAAACATTTCACTTGCCCCTTCCT |
| | | TCCCTGTCTCTGACAGGTCTGTCATTCTCGACCCTGATAGACTTGAGCCCCTTCT |
| | | GTTGGAGCAGTGGTTGGGATTGGAGTCCAAACCAACCATGTCAACAAAGATGA |
| | | AGCTACGTCCTCCTCCTGCTTATGTCCGTGGCTCGCTCCGGTCCACATTCGTCAT |
| | | GGGCCCATCTGATATTGCTAATGCTACACAATGGTTACAAACCCAGTGTGAGA |
| | | AGCTCAACAGATCATATCCTGTTCTCTTGTCACCCTACGTCGTCACTTGTGCCTT |
| | | TATATGGACCTGTTTTCTGAGAGCCCGAGTCCAGAACAGTGCTGTTACTAAAGC |
| | | CAAAGCAAAAGGCACCATGTACTTTGGATTTATTGCTGGTGGTATTACCCGTTT |
| | | ACCCTATCGGGTACCTGCTAAGTATCTTGGCAACTGTGTCGGGTTTGGACGGGC |
| | | AGCAGCGCAGAGGGAGGAGCTACTGAAGGAAGGTGAGGGGATGTTGGCAGCT |
| | | GCTGATGCAATTGGGCTAACCATTAAAAAGTTGGATAAAGATGTTTTAGGAGG |
| | | AGCTGAGAAATGGATATATGAATGGCAGACATTAATGGAATCCGAAGATCATA |
| | | TTCATGTGGTTGGGTCGCCCAAGGTGAACCTTTATGAGACGGATTTTTGGTGGG |
| | | GGAAACCGAAGAAGATAGAGGAAATTTCAACTGATGTTACCAGAGCCATCTCT |
| | | CTTACACAGAGCAGGGACATGAAAAGGGGAATTGAAATTGGCCTCACTTTACC |
| | | AAACTCCATTATGGATGACTTCTCCTCTATCTTCACTCAAGGCCTCCTTGTTTTT |
| | | CAAAATTAG |
| sp_074630_ | 113 | MAKSEQETMAKSEQETSIKLVSECFVKPKYEIKSAKQPYHLGPMDLVMLTIDPIQK |
| ygho_ | | GLVFTIKNSPLFLSSESHDNIEIIRTKVVSRLLEKLKHSLSIALVHFYPLAGRFTTQKQ |
| Spo04549_ | | PEHNTSLVFIDCNKGPGARFIHATSLDFTISDILSPVDVSIVHSFFDLGEKHVNYDCH |
| AT1 | | TKALLSIQVTELLDGVFIGFSMSHSVVDGTSFIHFVNTLSEIFKSDDFTTISRAPILNY |
| poly- | | RPCDIPILKFPFLDVEGFICRAYNPGPLRERIFHFSLNSMLRLKAMANQECGTQNVL |
| peptide | | SSFQALTAVVWRSITRVRNLPKDEQTTCFMAMGSRTRLNPPLSDDYFGNFMISTKF |
| | | ACKAEELLGNSLGWVAMNLRKIIMSTDEKSILATYKALADSPIVIPRETIPGPHGMT |
| | | RVIIGGSSRFDMYGPEFGLGRALAARMGYGNKDDGKITANPGCEGGGSVDLEICLR |
| | | PHIMASLEVDQEFMGFVS |
| sp_123780_ | 114 | MTPNLQIVTNGGKPENDEAEPVSPTGQYFNSKVLSVCVLAILEIDVPIDDSCVIPQLR |
| pgiy_ | | DVFLPMNPRFSSIMISDNKDVKQWKRVEVNLQDHVVVPSVPDGLSVESYDKYFDE |
| Spo21561_ | | YLTKITVDPLPQDRPLWELHVIKYPTSKAAGHFIWKLHHALGDGYTLMGVLLSGV |
| AT2 | | NRADDPSLPLTFPSTRSSSLVTNNKMNIISWVPRTFSAIYNGVYNFGWSFLKSTCKA |
| poly- | | DDKTPIRSGNEGLGFHPMKISTIELSLDQIKFIKTKLGATVNDILAGIIFLGVRKYMQ |
| peptide | | ATDTESGNSESTALVLFNTRNIGGYMTAEQMKKAQMKIWGNQFAFLHIAIPQLIND |
| | | KCSNPLDYVYEARKQISRFKSSPSVYLTAQCLELLGNAKDLRQQLNLSRVQRIKQA |
| | | Y |
| sp_149180_ | 115 | MLELAEDEVKNFFKVWAIVFASLSYCYYIGKLINPKGYTRLVAIIPIITLFLALPLNL |
| nwmy_ | | TSFHLGGMTCFFIAWLANFKLLLFAFDKGPLCANSSISFAKFLALSCLPIKIQHPPHK |
| Sp015788_ | | KSLKSHPSIYNYIIKGILLCLIIKIYDYGDYIHPKIIWLIFFFHSYFTIELVFAFLATSTNI |
| AT3 | | LLGLELEPQFNEPLISTSLQDFWGKRWNIMVTRILRPTVYLPTLEYSTKVVGRTWA |
| poly- | | TLPAVMSTFFVSAIMHELIFYYLGRNWPTFEVTWFFLLHGLCLCVEIVAKKLVGGK |
| peptide | | WRIPRWISGPATVLFVVGTGFWLFLPPLLKAGLDTRPFQEFAAVAKFVRSLKAALT |
| | | F |
| sp_198340_ | 116 | MAPPSSSSTTGSGNGSSFAVNIMASFYISPQQPSTTNSHSIPLTFFDIPWLQYPPLQPL |
| focw_ | | FFFQLPSTPQSSSSSSSSSSSSFDHNLYLEFSSTILPRLKHSLASALQYYFPFSGKLTTT |
| Spo13090_ | | THTIPNNLVFSTDSSDSVELTVSLCDADFNGLCSFLPRSTHLFQQLVPSLPNIESSNLT |
| AT4 | | TFPAPLLAIQITFFPTSSPGFSIGFASHPVLSDQRTFSNFLYSWASFSKFDNLNISLAPS |
| poly- | | FPVSDRSVILDPDRLEPLLLEQWLGLESKPTMSTKMKLRPPPAYVRGSLRSTFVMG |
| peptide | | PSDIANATQWLQTQCEKLNRSYPVLLSPYVVTCAFIWTCFLRARVQNSAVTKAKA |
| | | KGTMYFGFIAGGITRLPYRVPAKYLGNCVGFGRAAAQREELLKEGEGMLAAADAI |
| | | GLTIKKLDKDVLGGAEKWIYEWQTLMESEDHIHVVGSPKVNLYETDFWWGKPKK |
| | | IEEISTDVTRAISLTQSRDMKRGIEIGLTLPNSIMDDFSSIFTQGLLVFQN |

To our knowledge, SOAP10 (SEQ TD NO: 64) is the first member of the benzylalcohol acetyl-, anthocyanin-O-hydroxy-cinnamoyl-, anthranilate-N-hydroxy-cinnamoyl/benzoyl-, deacetylvindoline acetyltransferase (BAHD) superfamily of acyltransferases reported to be involved in triterpenoid saponin biosynthesis.

Table 16 below presents the amino acid and nucleotide sequences of the 10 enzymes, and genes encoding these enzymes, that comprise the biosynthetic triterpenoid saponin biosynthetic pathway producing Compound 11 (Yossoside V) in spinach.

TABLE 16
Nucleotide and Amino Acid Sequences for Triterpenoid Biosynthetic Pathway
Enzyme in Spinach

| | | SEQ | |
|---|---|---|---|
| | ENZYME | ID | |
| NAME | ACTIVITY | NO: | AA SEQ/NT SEQ |
| SOAP1 | saponin | 45 | atgtggaggttgaaggttggagaaggggctaatgacccatacttatatagcactaataactttgttg |
| | β-amyrin | | ggcgtcaaacttgggagtttgatcctaactatggcacccctgaggatattcaagaggtcgaagat |
| | synthase | | gctcgccgcgatttttacaataatcggtttaaagtgaagccttgtaacgatctcttatggcgttttcag |
| | | | ttcttaagagagaaaaacttcaagcaaaccatacctcaagtgaaggtgggtgacggggaggag |
| | | | atcacatatgagaccgcctcgacgacattaaagagagcggtgaatattttcacagccttgcagtct |
| | | | gaacatggccattggccggctgaaattgctggccctcagttcttccttcctcctttggtattttgctta |
| | | | tacattacaggagatcttaactctgttttcggaccagaacatcgtagagaaattcttcgcagcatcta |
| | | | ctatcaccagaacgaagatggaggttggggattacatattgaaggacatagcaccatgttctgta |
| | | | ccgcactgaattacatatgtttacgaatgcttggaataggacctgatgaaggtgatgacaacgcgt |
| | | | gccctagagcgcgtaaatggattctcgaccatggtagcgttacacatatcccttcttggggtaaaa |
| | | | cttggttatctatactgggcctgttcgattggtctggaagtaacccaatgccacctgagttctggatc |
| | | | cttcctacttttctccctatgcatccagcaaaaatgtggtgctactgtcgaatggtgtatatgccaatg |
| | | | tcatacttgtatgggaagagattcgtaggtccaatcacacctctcattaaacaacttagggaagaa |
| | | | ctctacaacgaaccctttgaacaaattagttggaagaaaatgcgacatttgtgtgcaccggaggat |
| | | | ctctactatcctcatccattgattcaagacttgatgtgggacgctctttacctttttacggaacctctcc |
| | | | tgacccgttggcctttcaacaagttgatacgaaagaaagcattagaggttacaatggaacacata |
| | | | cattatgaagatgagaacagtcgttacataacaattggatgtgtcgagaaggttttatgtatgttagc |
| | | | ctgttgggtggaagaccctaaaggggatcattacaagaaacatcttgcaagagtacaagattaca |
| | | | tttggattgctgaagatggattgaaaatgcagagttttggaagtcaacaatgggattgtgggttttca |
| | | | gtacaggcattattagcttctaatcttagtctcgacgaaattggacctgctcttaagaaaggccattt |
| | | | cttcattaaggagtcacaggtgaaggacaatccatccggcgacttcaaagctatgcatcgccata |
| | | | tctcaaagggatcgtggactttctccgaccaagatcatggttggcaagtctccgattgcactgccg |
| | | | aaggccttaagtgttgtctaatcttatcaacaatgcccccggaaattgttggagaaaagatggacc |
| | | | ctgaacgcctttatgattctgtcaatgtcttgctttctctacagagtaataaaggagggctagctgcc |
| | | | tgggaaccagcaggggctcaagaatggttggaggtcctaaacccaacagaattctttgaagaca |
| | | | ttgtgattgaacatgagtatgtagagtgtacggcttcagcaattcaagctttaataatgttcaagaag |
| | | | ttatacccaggacacaggaaaaaagagattgaaaattttgtagtaaacgcagtcaagtaccttgaa |
| | | | aacacccaatatcctagtggaggatggtatggaaattgggggatttgtttcatatatggaacatggt |
| | | | ttgcactaggagggctagcagcaggtgggaagacatactataattgtgctgctgttaggaagggt |
| | | | gttgattttttgcttactacacaaaaggaggatggtggttggggtgaaagttatatttcttgtcccaat |
| | | | aaggaatttgtgccaatagagggaaagtccaatttggttcagactggttgggctttgatgggtcta |
| | | | cttcatgctggacaggcggagagggatccaactcctctgcatcgtgcagcaaagcttttgattaat |
| | | | tcacaactcgaaaatggcgatttccctcaacaggaaataacaggagtcttcatgaagaattgcatg |
| | | | ttacattatccgatgtacagaagcatttatccactgtgggcaattgcagaatacagaaagcgtgttt |
| | | | cattaccttctatcaactctacttga |
| SOAP2 | cytochrome | 46 | atggaactcttctttatgtgtgggctagtccttttcctctccctatctctagcctccttcttccttttctata |
| | P450 | | accaccatagaacccgggggtacaagctacccccgggcaagatggggtggccggtggtggg |
| | | | cgagtcatttgaattttttcaaaccgggtggaaaggttacccggaaaagttcatatttgatagactg |
| | | | aacaagtacaccccaagccaagtgttcaagacttccatcgtaggagaaaaggttgcggttttatgt |
| | | | ggcgcggcgggtaacaagttcttgtactcaaacgagaacaagttagtacaagcttggtggccta |
| | | | gctctgttgataagatctttccttcttctacccaaacttcctccaaagaagaggctaagaagatgcg |
| | | | gaaactcctccctaacttcctcaagcccgaggctttacataggtacatacccatcatggatagcatt |
| | | | gccatccggcacatggagtccgggtgggagggaaaggacaaggtagaagtcttccctttggct |
| | | | aagaattacaccttctggctggcttgccgactcttcttaagcgtcgaggacccggctcatgtagcc |
| | | | aagttctccgaaccattcaacgacatagccgcagggatcatctcgatgccaatcgacctccccg |
| | | | gaacacccttcaaccgagggatcaagtcgtctaacgtcgtaaggaaagagttgagggccatcat |
| | | | aaagcagaggaaacttgacttagcagatggcaaggcttcacctacacaagatattctgtctcatat |
| | | | gttgttgacttgtactgaagatggcaagtttatgagtgaaatggatattgctgataagattctgggac |
| | | | ttcttattggtggacatgatactgctagtgcttcttgtacttttgttgttaagtttcttgctgagcttcctc |
| | | | acatatatgaaggtgtctacaaag agcaaatggagatagcaaattcaaaaaaagcaggagaact |
| | | | tctaaattgggaggacatacaaaaaatgaaatactcatggaatgtagcttgtgaagttatgcgtttg |
| | | | gctcctccacttcaaggtggtttcagggaagccctttctgatttcatgtataacggattccaaatccc |
| | | | caagggctggaagttatattggagtgcaaattcaacacatatgaacccggaatgcttcccggagc |
| | | | ccaagacgttcgacccatcgaggttcgacggtacgggaccagcaccatacacatacgtcccctt |
| | | | cggaggaggaccgagaatgtgcccgggcaaggagtatgcaaggctagagatattagtgttcat |
| | | | gcacaacgttgtcaagaggtttaaatgggaaaaaatgcttcctgatgagaaggttattgtcaatcc |
| | | | catgcctatcccagaacatggccttcctgtccgccttttccctcatcctogaactgtagctgcttaa |
| SOAP2 | | 47 | atggagttcttctttctgtgtggtctagtcttttacttctccatatctctagcctccttcttccttttctataa |
| -Like | | | ccaccatacaacccgggtttacccgctacccgcgggcgagatggggtggccggtggtgggcg |
| | | | actcgtttgaattttttcaaaccgggtggaacggttacccggaaaatttcatctttgatagactcaac |
| | | | aaatacaccccaagccaagtgttcaagactttcatcctaagagaaaaggttgtgttttatttgagaa |
| | | | ggttgtag |
| SOAP1 | saponin | 48 | MELFFMCGLVLFLSLSLASFFLFYNHHRTRGYKLPPGKMGW |
| | β-amyrin | | PVVGESFEFFQTGWKGYPEKFIFDRLNKYTPSQVFKTSIVGEK |
| | synthase | | VAVLCGAAGNKFLYSNENKLVQAWWPSSVDKIFPSSTQTSS |
| | | | KEEAKKMRKLLPNFLKPEALHRYIPIMDSIAIRHMESGWEGK |
| | | | DKVEVFPLAKNYTFWLACRLFLSVEDPAHVAKFSEPFNDIAA |
| | | | GIISMPIDLPGTPFNRGIKSSNVVRKELRAIIKQRKLDLADGKA |
| | | | SPTQDILSHMLLTCTEDGKFMSEMDIADKILGLLIGGHDTAS |
| | | | ASCTFVVKFLAELPHIYEGVYKEQMEIANSKKAGELLNWEDI |
| | | | QKMKYSWNVACEVMRLAPPLQGGFREALSDFMYNGFQIPK |
| | | | GWKLYWSANSTHMNPECFPEPKTFDPSRFDGTGPAPYTYVP |
| | | | FGGGPRMCPGKEYARLEILVFMHNVVKRFKWEKMLPDEKVI |
| | | | VNPMPIPEHGLPVRLFPHPRTVAA |
| SOAP2 | cytochrome | 49 | MWRLKVGEGANDPYLYSTNNFVGRQTWEFDPNYGTPEDIQ |
| | P450 | | EVEDARRDFYNNRFKVKPCNDLLWRFQFLREKNFKQTIPQV |
| | | | KVGDGEEITYETASTTLKRAVNIFTALQSEHGHWPAEIAGPQ |
| | | | FFLPPLVFCLYITGDLNSVFGPEHRREILRSIYYHQNEDGGWG |
| | | | LHIEGHSTMFCTALNYICLRMLGIGPDEGDDNACPRARKWIL |
| | | | DHGSVTHIPSWGKTWLSILGLFDWSGSNPMPPEFWILPTFLP |
| | | | MHPAKMWCYCRMVYMPMSYLYGKRFVGPITPLIKQLREEL |
| | | | YNEPFEQISWKKMRHLCAPEDLYYPHPLIQDLMWDALYLFT |
| | | | EPLLTRWPFNKLIRKKALEVTMEHIHYEDENSRYITIGCVEKV |
| | | | LCMLACWVEDPKGDHYKKHLARVQDYIWIAEDGLKMQSFG |
| | | | SQQWDCGFSVQALLASNLSLDEIGPALKKGHFFIKESQVKDN |
| | | | PSGDFKAMHRHISKGSWTFSDQDHGWQVSDCTAEGLKCCLI |
| | | | LSTMPPEIVGEKMDPERLYDSVNVLLSLQSNKGGLAAWEPA |
| | | | GAQEWLEVLNPTEFFEDIVIEHEYVECTASAIQALIMFKKLYP |
| | | | GHRKKEIENFVVNAVKYLENTQYPSGGWYGNWGICFIYGT |
| | | | WFALGGLAAGGKTYYNCAAVRKGVDFLLTTQKEDGGWGE |
| | | | SYISCPNKEFVPIEGKSNLVQTGWALMGLLHAGQAERDPTPL |
| | | | HRAAKLLINSQLENGDFPQQEITGVFMKNCMLHYPMYRSIYP |
| | | | LWAIAEYRKRVSLPSINST |
| SOAP2 | | 50 | MEFFFLCGLVFYFSISLASFFLFYNHHTTRVYPLPAGEMGWP |
| -Like | | | VVGDSFEFFQTGWNGYPENFIFDRLNKYTPSQVFKTFILREK |
| | | | VVFYLRRL |
| SOAP3 | cytochrome | 51 | atgatagaaatcgggtatattgtaaaatgggtaatttgtttagtgattgttagatgggtatggaagatt |
| | P450 | | gtgaattgggtttggtttacaccaaaaaggcttgagaagtttctaagaaaacaaggtttagatgga |
| | (C-2 | | aattcatacagatttttgttgggtgatctcaaagatatgtctaaaatgcgtaaagaagctagacaaa |
| | hydroxylase) | | aacctattccttttactcatgacttctttcatcgtatcttgcctttccacaatcaccatttcaataaatacg |
| | | | gggaaagcttcttttcatggatggggcctataccagttgtgaatgttgcagaacaagagcaagtaa |
| | | | agggtgtgttcactaggataaaagagtttcagaaggccaaattaaacccacttgttgcattgcttgt |
| | | | ccctggacttgtgagcgctgaaggtgataaatgggtcaagcacaggaagctcatcaacccggct |
| | | | tttcatatggaaaagcttaagcttatgcatccagcatttggcgccagtgttttggatatggtgaacaa |
| | | | gtgggagaagatagtatctaaaacaggttcctctgaagtggatgtgtggccgtttgtttccagcct |
| | | | gactgcagatgctatctctcgtgctgcttttggcagtagctatgatgaaggaagaaagatatttgag |
| | | | ttggttcttgaacaaactgaaatcaccctacgccttctgcaatcagtttatatccctggatggatgtat |
| | | | gtgccaacaaagaccaacaggaggatgaaaacagtaaactctgaaatacaaaatttattaaccg |
| | | | ggataatcgttaagagaaagaagg caatggaggccggcgaagctgccaaggatgatttgttgg |
| | | | ggatattgttggagtccaactacaaagatactgaaaatgttctcagtaataagaaaaaactaagca |
| | | | tgactttccaggaattgattgatgagtgcaaactgttctacttagcagggcaagagtcgacctcgg |
| | | | tgttgctagcatggacaatgattctgttgggaaagcacacagagtggcaagcacgagcacgaga |
| | | | agaagtagttgcaacgtttggtaaaaacgaacctgattttgaaggcttaaaccatttgaagatagtg |
| | | | acaatgatactgaatgaggtgttgaggttgtaccctccagtgtgtacaatcacccgtaagaatttca |
| | | | accacgacgtacagcttggaaatctgacagtccctcgtggtgctatggttacgatgtcagcatatc |
| | | | gtattcaaagagatcctaaaatatggggtgatgatgcaaaagagtttaacccacagagattttcag |
| | | | aaggggttgcaaaggctacaaaggggaatattgcattctttccgtttggttgggggccgcgaattt |
| | | | gcatcggacagaactttgcacttattgaagctaaaatggcagtgtccatggttttacaacgcttttct |
| | | | tttgagctatcaccgtcttatactcatgctcctaccactatcctcactcttcaaccccaacaaggtgct |
| | | | catctcattatacataagctcagggactaa |
| SOAP3 | cytochrome | 52 | MIEIGYIVKWVICLVIVRWVWKIVNWVWFTPKRLEKFLRKQ |
| | P450 | | GLDGNSYRFLLGDLKDMSKMRKEARQKPIPFTHDFFHRILPF |
| | (C-2 | | HNHHFNKYGESFFSWMGPIPVVNVAEQEQVKGVFTRIKEFQ |
| | hydroxylase) | | KAKLNPLVALLVPGLVSAEGDKWVKHRKLINPAFHMEKLK |
| | | | LMHPAFGASVLDMVNKWEKIVSKTGSSEVDVWPFVSSLTAD |
| | | | AISRAAFGSSYDEGRKIFELVLEQTEITLRLLQSVYIPGWMYV |
| | | | PTKTNRRMKTVNSEIQNLLTGIIVKRKKAMEAGEAAKDDLL |
| | | | GILLESNYKDTENVLSNKKKLSMTFQELIDECKLFYLAGQES |
| | | | TSVLLAWTMILLGKHTEWQARAREEVVATFGKNEPDFEGLN |
| | | | HLKIVTMILNEVLRLYPPVCTITRKNFNHDVQLGNLTVPRGA |
| | | | MVTMSAYRIQRDPKIWGDDAKEFNPQRFSEGVAKATKGNIA |
| | | | FFPFGWGPRICIGQNFALIEAKMAVSMVLQRFSFELSPSYTHA |
| | | | PTTILTLQPQQGAHLIIHKLRD |
| SOAP4 | cytochrome | 53 | atgatttcaaagagcgcgagtggtgttgcaattgcttcattgggttgcattatcatactgtattggatg |
| | P450 | | tggaaattactaaaagggctatggttaacaccaaaaaagctagagaaatgtctaaaacaacaag |
| | (C-23 | | gtcttgttggtaattcctacaaatttttgattggagatatgaaagaaagctcaaaattgcgtaacgaa |
| | oxidase) | | gctttacaaaaacccattcctttcactcatgattattacaaccgtattcagcctttcatccatcagattc |
| | | | tcaacaattctggtgcaggcaaaaatatctacacatggttgggaccagtgccaacaatactaatta |
| | | | cacaacctgagttaataaaggatgctttcaataggatgaacaattttcagaaaccaagattaaatcc |
| | | | atatactcaaatgctttcaactggacttccgaactatgagggtcagaaatgggctaaacacagga |
| | | | agcttctcaaccctgcttttcaacttgataagctcaagcttatgatccatacttttgaaacctgtgttac |
| | | | ggatacactgaataagtgggagaagctagtttgtaaaacaggttcttcagaggttgatatatggcc |
| | | | atatttgacaactttaacgggagatggtattgctagagctgcatttggaagtagctttgaagatgga |
| | | | agaagaatattcgagcttctcacactgcagaaggatattgttattagtcttctcaaatattcttatattc |
| | | | caggatttaaatatatgccaataaagggtaaccggaagatgaaagaagcggacaatgaaataaa |
| | | | acctctgttgacgaatataattaatagaaggaggaaagcgatggaggccggagaagctcccaa |
| | | | agacgacttgttagggatgctacttgaatccaatgcaaacgaggctcgacaagttaatgaaaatg |
| | | | aaagtggtagtagcaagcgaaaatctgatctaacgatgagcttccctgagatgatcgatgcttgc |
| | | | aagcagttcttcttggctggtcaagagaccacctcagtggccctaacatggacaatgcttttgttag |
| | | | ccaagcaccaagattggcaaacacgagctcgacaagaagtacttgctacatttggaatgaatac |
| | | | cccagactttgatggcatacataatcgtcttaagattgtgacaatgatactctacgaggtgttaaggt |
| | | | tgtatccgccagtccctgcaacatcgcgaagggttcatgatcgtgaaacaaagctaggagatttg |
| | | | gtaataccacaaggggtaggagtttcattttccatacttcatgcacacttgaaccctgaaatttggg |
| | | | gtgatgatgccaaagaattcaagcctgatagatttgcagaagggattgcaaaagcaacaaaagg |
| | | | gaataactcttacttcccctttggttggggacctaggatttgcattggccaaaacttcgcactagttg |
| | | | aggcgaaaatggcattgtgtatgattttgcagcgtttctctttcgatctctcgccttcatacatccatg |
| | | | ctccgactagtctcatatcccttcaacctcagcatggtgcccacattattttacatcgattttaa |
| SOAP4 | cytochrome | 54 | MISKSASGVAIASLGCIIILYWMWKLLKGLWLTPKKLEKCLK |
| | P450 | | QQGLVGNSYKFLIGDMKESSKLRNEALQKPIPFTHDYYNRIQ |
| | (C-23 | | PFIHQILNNSGAGKNIYTWLGPVPTILITQPELIKDAFNRMNNF |
| | oxidase) | | QKPRLNPYTQMLSTGLPNYEGQKWAKHRKLLNPAFQLDKL |
| | | | KLMIHTFETCVTDTLNKWEKLVCKTGSSEVDIWPYLTTLTG |
| | | | DGIARAAFGSSFEDGRRIFELLTLQKDIVISLLKYSYIPGFKYM |
| | | | PIKGNRKMKEADNEIKPLLTNIINRRRKAMEAGEAPKDDLLG |
| | | | MLLESNANEARQVNENESGSSKRKSDLTMSFPEMIDACKQF |
| | | | FLAGQETTSVALTWTMLLLAKHQDWQTRARQEVLATFGMN |
| | | | TPDFDGIHNRLKIVTMILYEVLRLYPPVPATSRRVHDRETKLG |
| | | | DL VIPQGVGVSFSILHAHLNPEIWGDDAKEFKPDRFAEGIAK |
| | | | ATKGNNSYFPFGWGPRICIGONFALVEAKMALCMILQRFSFD |
| | | | LSPSYIHAPTSLISLQPQHGAHIILHRF |
| SOAP6 | Glycosyl | 55 | atgacgggaaaaggaagaacgatggaggtgatcatgatgccatttcaccaccaaggtcacttaa |
| | transferases | | ccccgatgctccaattcgcgaagcgcttcgcttggaaaggtgctggctcgatccggatcaccctc |
| | (UDP- | | gccaccaccctctccacegcccaaaatatgaccaattccaaaaacaacaacaacaataacgatt |
| | glycosyl- | | acgatttcctgacggtcgaaagcatctacgacgataccgatgattctcagctcaaattcatgggtc |
| | transferase; | | gtatgggaaagttcaagtccgaagcctcactccaacttggccgtctaatcactactaagagcatc |
| | Fucosyl | | gacaataataaatgtatgctcgtttatgatgcgtatctgccttgggcactggatgtgggcaaggac |
| | transferase) | | cataacatacaggctgcggctttcttcgtccaggcttgtgcgtatatggcatccttttaccctatgtttt |
| | | | tagaggaatttgggtcggatgatcaacatcctgttgttgcggctgctaaggctgaatctgttcctag |
| | | | tttgtcggttgagctgccgtcgcgggaggaaatggaacgatacgcgccgaaatgtgcacaatcc |
| | | | ccgagttctgatgataaacccaatactgttaagaaatcgcttcaccctgtctaccggatggtggttt |
| | | | catcaattacaacccttcatcttgctgatttcgtgctcatcaactcctttgatcaccttgaacatcagct |
| | | | ggatgtcttagcacatgaagcagtagggtgtttcataacccattgcggttggaactcgataattga |
| | | | ggcgaccaactttggggttccgatgttggggatgccacagttcatggaccagtttttggatgctca |
| | | | ttttatggagaaggtttggggtgttggaattagggctaaggctgatgagaaaaactttgttacttgtg |
| | | | acgaaatcaagtgcggtgtcaatgaaattatgtacggagataaggcaaatatgatcaaggagaat |
| | | | gcagcaaagtggaaagacttggctaaggaggcagttggtgaaggaggcagttcagataagaat |
| | | | atcgacgagatcattaactggcttgcgtcctcctaa |
| SOAP6 | Glycosyl | 56 | MTGKGRTMEVIMMPFHHQGHLTPMLQFAKRFAWKGAGSIR |
| | transferases | | ITLATTLSTAQNMTNSKNNNNNNDYDFLTVESIYDDTDDSQL |
| | (UDP- | | KFMGRMGKFKSEASLQLGRLITTKSIDNNKCMLVYDAYLPW |
| | glycosyl- | | ALDVGKDHNIQAAAFFVQACAYMASFYPMFLEEFGSDDQHP |
| | transferase; | | VVAAAKAESVPSLSVELPSREEMERYAPKCAQSPSSDDKPNT |
| | Fucosyl | | VKKSLHPVYRMVVSSITTLHLADFVLINSFDHLEHQLDVLAH |
| | transferase | | EAVGCFITHCGWNSIIEATNFGVPMLGMPQFMDQFLDAHFM |
| | | | EKVWGVGIRAKADEKNFVTCDEIKCGVNEIMYGDKANMIK |
| | | | ENAAKWKDLAKEAVGEGGSSDKNIDEIINWLASS |
| SOAP7 | Glycosyl | 57 | atgggtaaaacagtagcagcttcagagcagttacacatagtaatgatcccatggtttgcttatgga |
| | transferases | | cacattcttccctattttgagctttcaaacaaacttgctgaaaaaggccataaaatcaccttggtagtt |
| | (UDP- | | ccaaacaaagtcaaacttgatttagaaccaaagatccgtcatccttccttaatcagcttacatgcatt |
| | glycosyl- | | cactgtcccacacattgaacccttacctccggggactgagacatgttcagacgtccccattgaact |
| | transferase) | | tcagcaccaccttgctgttgccatggacagggcccggcctgaggtggagtccatcatatcagcc |
| | | | attgacgatccgaagccggatctgttgttctacgataacgcttactgggtgcctgagatagccaca |
| | | | aagctggggatgaagtctgtgttttaccagattgcatgtgctttaagtatcacccgcattaagcaaa |
| | | | ccccgagtgcgagtgcgagtgcgagtgcgagtgccaagctcttcactttacccaagtgggtgct |
| | | | gacgcctaaggtgttaggcgacgcaagagccaattatggagaagggatcacctattaccagag |
| | | | agtgaagaaggctctaagttcctgtgatgctatcgccttacgcacatgccgggagattgaagggg |
| | | | aatcctctgatatcctggctgcacaatataacaagccagtcttcttaacaggtccggtcctacccga |
| | | | ggttgaattcctcccccctttggacaattcctgggctgagtggctagccaagtttgggcctaagtc |
| | | | cgtggttttatgttgcttcggaagccagtacgtccctgacaaggctcaactacaggagatggccct |
| | | | tgcccttgaggatactggtcttcccttcttgatgtccgttaagccacccacagagtgcgccaccata |
| | | | gaggaggcgttgccagaagggttctcagagagagtcaaggaacgcggggtggttcatggtgg |
| | | | atgggtgcaacagctacaaatactagctcacccatcagtgggttgctttatttgtcattgtgggtac |
| | | | gggtcaatgtgggaggggttgttgagtgataaccagctagtcttattaccacagcttcctgaccag |
| | | | ttaatgatggctcaaatgttggcagaaaagctcaaggtgggtgtgatggtggacagagaagaag |
| | | | atgatggggggtttccaggaagaacttgtgccaagcagtcaagtctgtcatggatcctcattccg |
| | | | agtttgcagctttactcaagaacaaccatgctaacttcagagacaagttgctaaccaacggttttat |
| | | | ggctaattaccttgaagtttttgaccaggatttgaaacgctttcttactgcaaattaa |
| SOAP7 | Glycosyl | 58 | MGKTVAASEQLHIVMIPWFAYGHILPYFELSNKLAEKGHKIT |
| | transferases | | LVVPNKVKLDLEPKIRHPSLISLHAFTVPHIEPLPPGTETCSDV |
| | (UDP- | | PIELQHHLAVAMDRARPEVESIISAIDDPKPDLLFYDNAYWV |
| | glycosyl- | | PEIATKLGMKSVFYQIACALSITRIKQTPSASASASASAKLFTL |
| | transferase) | | PKWVLTPKVLGDARANYGEGITYYQRVKKALSSCDAIALRT |
| | | | CREIEGESSDILAAQYNKPVFLTGPVLPEVEFLPPLDNSWAE |
| | | | WLAKFGPKSVVLCCFGSQYVPDKAQLQEMALALEDTGLPFL |
| | | | MSVKPPTECATIEEALPEGFSERVKERGVVHGGWVQQLQILA |
| | | | HPSVGCFICHCGYGSMWEGLLSDNQLVLLPQLPDQLMMAQ |
| | | | MLAEKLKVGVMVDREEDDGWVSRKNLCQAVKSVMDPHSE |
| | | | FAALLKNNHANFRDKLLTNGFMANYLEVFDQDLKRFLTAN |
| SOAP8 | Glycosyl | 59 | atgggtggagagaaagagttgcggatagtgatgttcccatggcttgcctttggacattttatcccat |
| | transferases | | accttcacctttcaaacaaacttgctgaaaaaggccacaaaatcaccttgttgcttcccaacaaag |
| | (UDP- | | ctaggcttcagttggagtcacttaaccttcatccttctctcataactttccattcaattactgtcccacc |
| | glycosyl- | | cctcgaaactctcccttatggcactgaaacaactgcggatatctccctcgaccaacatggtgaact |
| | transferase) | | ctcgatttccatggaccgcactcggcccgaggtggagtctttcctatcaacccataagcccgacc |
| | | | tcgtcctctacgacatggcccattgggtacccgagattgctgctaaggtcgggattaagtcagttt |
| | | | catacaacgttgtatgtgctattgctgtatctcatgttagacctagcctccctcttccaaaaggaacg |
| | | | gcagcacatgtacccctgccattgtcgtctgtccctaagtggagtcttaatcag cacggttcatcaa |
| | | | caccatattttggggaagggataacgttacttgaacggtctgtaatctccctctcgtctgcggatgc |
| | | | aatagccatccgcacgtgcagggagattgaaggggtatattgtgaccgtgttgctgccacattca |
| | | | acaagcctgtccttgtcaccagccacgccttgcctgatcttgaactegaactctctccgttggaga |
| | | | ctcgctgggccgagtggctagctaggttcgagccagggtcagtgatcttttgctgccttggtagtc |
| | | | agcatgtcttagacgcaccccaactgcaagagttggccctggggttggaaatgacaggactacc |
| | | | cttcttgatggctgtaaaaccccctgtggggtgtacctccttggaggaggtgcttccagaagggtt |
| | | | taatgatcgggttagcgggcgaggggtggttcacggtgggtgggtgcagcagcagcagataat |
| | | | ggcgcacccatcgttagggtgctttgtgaccctttgtgggtcttcgtcgatgtgggaggggttagt |
| | | | gagtgaaagtcagttggtattactcccacaactggcagaccaaactctgtatgccaagttaatggc |
| | | | agatgagctcaaggtgggtgtgaaggtggagagagaagagaacgggtggatgacgaagcga |
| | | | agtctatgtgaagctatcaagagtgtgatggatgaagatagtgatataagtcatgtagttaggaaa |
| | | | aatcatgctaaatatagaagtatgttgattagccctggctttattagtggctacattgacaacttcatc |
| | | | aaggatttacaagcccttgttccttag |
| SOAP8 | Glycosyl | 60 | MGGEKELRIVMFPWLAFGHFIPYLHLSNKLAEKGHKITLLLP |
| | transferases | | NKARLQLESLNLHPSLITFHSITVPPLETLPYGTETTADISLDQ |
| | (UDP- | | HGELSISMDRTRPEVESFLSTHKPDLVLYDMAHWVPEIAAKV |
| | glycosyl- | | GIKSVSYNVVCAIAVSHVRPSLPLPKGTAAHVPLPLSSVPKW |
| | transferase) | | SLNQHGSSTPYFGEGITLLERSVISLSSADAIAIRTCREIEGVYC |
| | | | DRVAATFNKPVLVTSHALPDLELELSPLETRWAEWLARFEP |
| | | | GSVIFCCLGSQHVLDAPQLQELALGLEMTGLPFLMA VKPPV |
| | | | GCTSLEEVLPEGFNDRVSGRGVVHGGWVQQQQIMAHPSLG |
| | | | CFVTLCGSSSMWEGLVSESQLVLLPQLADQTLYAKLMADEL |
| | | | KVGVKVEREENGWMTKRSLCEAIKSVMDEDSDISHVVRKN |
| | | | HAKYRSMLISPGFISGYIDNFIKDLQALVP |
| SOAP9 | Glycosyl | 61 | atggagctttcaaaccctaccacaacccctaccttaaacgcaacccaacccttacgaggctatttc |
| | transferases | | attccattaatcacagttccaagccacatttccaatcttgttgacattgctaaactcttctcatcacgg |
| | (UDP- | | ggagtacatgtgactatcctcaccacccaccacacctccctccgcttcaaacaatccatacatgat |
| | glycosyl- | | tggggcttcaaaatcgacctccacatcgtcgacttcccgttcagggaagtcggcttaccggaagg |
| | transferase; | | agtggaaaattacagtgatgccacccctgagcaagcaagccagcttttccaggcctttatgatgct |
| | Xylosyl | | tcagaagcctatggaggatgccattcgggctgctaagcccgacttcategtttccgataggtattat |
| | transferase) | | cattggtctactgatcttgcacgtgagcttgctattccacggctcatctttcatgtcagatgttattttg |
| | | | cattgtgtgctgctgaggttgttgccaagtttgcccctcatgagaaggttgaatctgacactgacct |
| | | | ctttttccttcctgacctccctgataccatccacatgacccgtttgcagcttcccgaatggattcaga |
| | | | cccgaaacatgttcactgttctcaatgagagaatggacgaggccgatagggagtgctacggtgtt |
| | | | attgtaaacagctgctacgagttggagagagcttatgctgacttctaccgcagcaacttgggtcga |
| | | | cgtgcttggtgtatcggtccctatccggtacactgcgacaaggttggtaaaaagaaaggagatga |
| | | | cagcaaaaaacattcatgttttgaatggcttgataaaatgggagaaggagaagttatatacgtgag |
| | | | ttttggcactctgtcgtgtttcagccctgctcaaatctcagagctggctactgcactcgaaatgtctg |
| | | | gtcacccgtttatctgggtagtaaggaatggtgagaaattgttacctgatggatttgaagaaagaat |
| | | | tacagagcaggacaaaggggtgttaataaaagactgggcgccacaagtgaaaatacttgagca |
| | | | cccagctgtaggcggatttctgactcattgtggatggaactcaactgtagaaagtttagcagcagg |
| | | | tgtgccaatggtcacatggccgcttggtgccgagcaattcttcaatgaaaagttgattagtggagtt |
| | | | ttgaaggtgggggtcgaggtcgggtctgagaagtggagtaggggtatagtaccgaatactgata |
| | | | tgattgagaaggataaaatagaaagggcgattaaagagctgatgagtaaagaacccgaggccg |
| | | | aggaaaggaggcagaaggttaaggagttgagtaaggctccaagaaatgcggttgaagaaggt |
| | | | ggttcgtctcgtaacaatttaagtgatttgattgaaaaattacaacgtttaaaggcgaatgaaatatc |
| | | | agtgtccacaaactcagaataa |
| SOAP9 | Glycosyl | 62 | MELSNPTTTPTLNATQPLRGYFIPLITVPSHISNLVDIAKLFSSR |
| | transferases | | GVHVTILTTHHTSLRFKQSIHDWGFKIDLHIVDFPFREVGLPE |
| | (UDP- | | GVENYSDATPEQASQLFQAFMMLQKPMEDAIRAAKPDFIVS |
| | glycosyl- | | DRYYHWSTDLARELAIPRLIFHVRCYFALCAAEVVAKFAPHE |
| | transferase; | | KVESDTDLFFLPDLPDTIHMTRLQLPEWIQTRNMFTVLNERM |
| | Xylosyl | | DEADRECYGVIVNSCYELERAYADFYRSNLGRRAWCIGPYP |
| | transferase) | | VHCDKVGKKKGDDSKKHSCFEWLDKMGEGEVIYVSFGTLS |
| | | | CFSPAQISELATALEMSGHPFIWVVRNGEKLLPDGFEERITEQ |
| | | | DKGVLIKDWAPQVKILEHPAVGGFLTHCGWNSTVESLAAGV |
| | | | PMVTWPLGAEQFFNEKLISGVLKVGVEVGSEKWSRGIVPNT |
| | | | DMIEKDKIERAIKELMSKEPEAEERRQKVKELSKAPRNAVEE |
| | | | GGSSRNNLSDLIEKLQRLKANEISVSTNSE |
| SOAP | Acyl | 63 | atgggggaagtcaaccatgaagaagtagaaattgaaataatatcaatagaaaccataaaaccatc |
| 10 | transferase | | atcactacttccaccaaaaactcctccaaaaaccatcacactttctcacctcgatcaagctgcccct |
| | (BAHD | | ttgtactactatcctttacttttatactacactaacactactactactaccccaacatcacaaattcgag |
| | acyl | | ttgacataacaagtaccctaaaaacttcacttagcaaaacacttgacaaattccaccctattgcagg |
| | transferase) | | tcgatgtgtggacgactctacaatttgttgcaaccaccaaggaataccattcattgaaaccaaagtt |
| | | | gactccaatatcttggatgtcatgaactcgcctgagaaaatgaagttgcttatcaagtttctccctca |
| | | | tgcagagtttcaagatgtgactcgaccagtctcggatttaaaccatttggcgtttcaagtcaatgtttt |
| | | | ccggtgtggtggggtgatcattggctcctatgtgctccacaagctccttgatggaatctctcttgga |
| | | | actttctttaaaaattggtcaaccattgctaatgatgagcgagttaaggacgacgacctagtacaac |
| | | | ctgactttgaagccactattaaggcgttccctccgcgtacagcaactccaatgcttcctcgtaatca |
| | | | acaacttccaaaggcggctgaaaaaccaaataataatccagtcaaagttcttgtgacaaagagct |
| | | | tcgtatttgacattgtttctttaaagaagatgatgttcatggctaagagtgaattggttcctaaaccca |
| | | | ccaaatttgagaccgtgacagggtttatttgggaacaaaccttatcaacattgcgtaattctggagtt |
| | | | gaagttgaacatacatcgcttataatacctgtaaacatccgcccaaggatgagtccgccactccc |
| | | | aagaggatccatgggtaacttgctcaagaatgcaaaggcacaggccaacaccagcagcagca |
| | | | atgggcttcaagaccttgttaaagaaatccattcatctttgtctcaaacaacccagaaaattaatact |
| | | | cctcctcctcctcctcctcctcctcctactactactgctacaacaatccattcatctttgtctcaaacaa |
| | | | cccagaaaattaatactcctcctcctactactacaacaatccattcatctttgtctcaaacaacccag |
| | | | aaaattaatactactactactacagcagaggttattttgactaaacggaaagttgacaatccagtta |
| | | | cacagaatcgagaaggaaactacctcttcaccagttggtgcaagattgggttggatgaggctga |
| | | | cttcgggttcggaaagcccgtttgggtaattcccaacgatgggagaccccctaaggtcaggaat |
| | | | atgattttccttactgattataggcatcccgaaacaggcgttgaaggaattgcagcatggattacgt |
| | | | tggaagagaaacaaatgcaatgtttaaagtcaaacccagaattccttgcttttgctactcctaattag |
| SOAP | Acyl | 64 | MGEVNHEEVEIEIISIETIKPSSLLPPKTPPKTITLSHLDQAAPL |
| 10 | transferase | | YYYPLLLYYTNTTTTTPTSQIRVDITSTLKTSLSKTLDKFHPIA |
| | (BAHD | | GRCVDDSTICCNHQGIPFIETKVDSNILDVMNSPEKMKLLIKF |
| | acyl | | LPHAEFQDVTRPVSDLNHLAFQVNVFRCGGVIIGSYVLHKLL |
| | transferase) | | DGISLGTFFKNWSTIANDERVKDDDLVQPDFEATIKAFPPRT |
| | | | ATPMLPRNQQLPKAAEKPNNNPVKVLVTKSFVFDIVSLKKM |
| | | | MFMAKSELVPKPTKFETVTGFIWEQTLSTLRNSGVEVEHTSL |
| | | | IIPVNIRPRMSPPLPRGSMGNLLKNAKAQANTSSSNGLQDLV |
| | | | KEIHSSLSQTTQKINTPPPPPPPPPTTTATTIHSSLSQTTQKINTP |
| | | | PPTTTTIHSSLSQTTQKINTTTTTAEVILTKRKVDNPVTQNRE |
| | | | GNYLFTSWCKIGLDEADFGFGKPVWVIPNDGRPPKVRNMIFL |
| | | | TDYRHPETGVEGIAAWITLEEKQMQCLKSNPEFLAFATPN |
| SOAP5 | Cellulose | 65 | atggcaacttctcacattcgcaatgtccaattaaccagagccattgttaaccgtctccacatcttcct |
| | synthase | | ccattccgtagccatcttctcgctcttctactaccgtttcacttccttcttcaactccgacatctccata |
| | Like G | | cttgcttactccttactcaccaccgccgaactcttcttaacctttctatgggcttttactcaggctttcc |
| | (glucuronic | | ggtggcgtcccgtaatgagggaagtctccgggtacgaatccatcaaacccgaacaactaccgg |
| | acid | | gtttggatgtcttcattgtcactgctgacccgacaaaggagccagttctggaggtgatgaactccg |
| | transferase) | | tgatatcatccatggctttggattatccggttgatagactggcggtttacttgtcggatgacggtggt |
| | | | tctccgttgtcgaaggaggcgattaagaaggcttatgagtttgctaagctttggattcctttttgtaat |
| | | | aagtataatgttaagacaaggtgtcctcaggctttcttctcgcctcttgctgatggggaaaggcttg |
| | | | attggaattctgagtttatggctgatcaattggaactccagaccaaatatgaagcttttagaaactat |
| | | | gtggagaaagaaagtggagataacaccaaatgtactgcagttcatgatcgacctccttgcgttga |
| | | | gattatacatgacaacaaacagaacggagaaagtgatgtgaagatgccccttctggtttatgtagc |
| | | | cagggaaaagagacctggtcgtcctcatcgtttcaaagctggagcccttaatgctcttcttcgagt |
| | | | atccagtttaatgagcaatgcaccttacttattggtgttggattgtgatatgtactgccatgatccaac |
| | | | ttctgctcgtcaatctatgtgcttccatcttgacacaaacatggcttcctctcttgcatatgtgcaatac |
| | | | cctcaaattttctataatgttagcaaaaatgacatctatgatggccaagccagatcagctcatatga |
| | | | cgaaatggaaaggcatggatggactcagaggcccggtcttgaatggaactgggtattatttgaa |
| | | | gcgaaaagcattatttggaaagcctaataacgaagatgaatacctcaacagtcaaccagaaaag |
| | | | gcctttggctcctccacaaaattaattgctgcactaagagagaactccaagcaaaatcttgccata |
| | | | aaggaattgacagaagatgagttgtaccaagaggctagaaatttggctacttgcacatatgaagc |
| | | | aaacacactatggggcagtgaggtaggatattcgtatgagtgcttgttggagagtacattcactgg |
| | | | atatatgttacattgcagaggatggaaatctgtgtatctttacccaaaaagaccatgcttcttgggat |
| | | | gcacaacgattgatatgaaggatgctacggttcaactaataaaatggacctcctcattacttggaat |
| | | | tgccctgtcgaagtctagccctctaactttggccatgtccagtatgtcaatcctgcaaagcatgtgtt |
| | | | acgcgtacatcacatttacaggcctttttgcagctccattggttatatatggtgttgtccttccaataa |
| | | | gcctattgaagggctttcctattttccctaaggtatcggatccatggattttgccatttgtgttgatattt |
| | | | gtatcctcccatcttcaacatctatatgaggtcctggaaagtgacaaatcagcaacacaatggtgg |
| | | | aatgaggtgagaatttggatgatgaaatcagtgacagcctgtttgtttgggttgacggaagcgata |
| | | | atgaagaagattggagtacaaactgcaacattcagattaacaaataaggtagttgagaaggaaaa |
| | | | gatggataaatacgagaaggagaggtttgatttctcaggagcagctatgcttatggttcctcttaat |
| | | | attttggtggtactaaatatggtgtcattcattggtggactcatgagggtcataatcaacaacagttat |
| | | | gatcaaatgtttgcacaacttttcctctccttttttgtcctacttcttagctaccctgttgttaagggatg |
| | | | gttataa |
| SOAP5 | Cellulose | 66 | MATSHIRNVQLTRAIVNRLHIFLHSVAIFSLFYYRFTSFFNSDI |
| | synthase | | SILAYSLLTTAELFLTFLWAFTQAFRWRPVMREVSGYESIKPE |
| | Like G | | QLPGLDVFIVTADPTKEPVLEVMNSVISSMALDYPVDRLAVY |
| | (glucuronic | | LSDDGGSPLSKEAIKKAYEFAKLWIPFCNKYNVKTRCPQAFF |
| | acid | | SPLADGERLDWNSEFMADQLELQTKYEAFRNYVEKESGDNT |
| | transferase) | | KCTAVHDRPPCVEIIHDNKQNGESDVKMPLLVYVAREKRPG |
| | | | RPHRFKAGALNALLRVSSLMSNAPYLLVLDCDMYCHDPTSA |
| | | | RQSMCFHLDTNMASSLAYVQYPQIFYNVSKNDIYDGQARSA |
| | | | HMTKWKGMDGLRGPVLNGTGYYLKRKALFGKPNNEDEYL |
| | | | NSQPEKAFGSSTKLIAALRENSKQNLAIKELTEDELYQEARN |
| | | | LATCTYEANTLWGSEVGYSYECLLESTFTGYMLHCRGWKS |
| | | | VYLYPKRPCFLGCTTIDMKDATVQLIKWTSSLLGIALSKSSPL |
| | | | TLAMSSMSILQSMCYAYITFTGLFAAPLVIYGVVLPISLLKGF |
| | | | PIFPKVSDPWILPFVLIFVSSHLQHLYEVLESDKSATQWWNEV |
| | | | RIWMMKSVTACLFGLTEAIMKKIGVQTATFRLTNKVVEKEK |
| | | | MDKYEKERFDFSGAAMLMVPLNILVVLNMVSFIGGLMRVII |
| | | | NNSYDQMFAQLFLSFFVLLLSYPVVKGWL |
| SOAP5 | Cellulose | 93 | ATGGCAACTTCTCACATTCGCAATGTCCAATTAACCAGAG |
| | synthase | | CCATTGTTAACCGTCTCCACATCTTCCTCCATTCCGTAGCC |
| | Like G | | ATCTTCTCGCTCTTCTACTACCGTTTCACTTCCTTCTTCAAC |
| | (glucuronic | | TCCGACATCTCCATACTTGCTTACTCCTTACTCACCACCGC |
| | acid | | CGAACTCTTCTTAACCTTTCTATGGGCTTTTACTCAGGCTT |
| | transferase) | | TCCGGTGGCGTCCCGTAATGAGGGAAGTCTCCGGGTACGA |
| | | | ATCCATCAAACCCGAACAACTACCGGGTTTGGATGTCTTC |
| | | | ATTGTCACTGCTGACCCGACAAAGGAGCCAGTTCTGGAGG |
| | | | TGATGAACTCCGTGATATCATCCATGGCGTTGGATTATCC |
| | | | GGTTGATAGACTGGCGGTTTACTTGTCGGATGACGGTGGT |
| | | | TCTCCGTTGTCGAAGGAGGCGATTAAGAAGGCTTATGAGT |
| | | | TTGCTAAGCTTTGGATTCCTTTTTGTAATAAGTATAATGTT |
| | | | AAGACAAGGTGTCCTCAGGCTTTCTTCTCGCCTCTTGCTGA |
| | | | TGGGGAAAGGCTTGATTGGAATTCTGAGTTTATGGCTGAT |
| | | | CAATTGGAACTCCAGACCAAATATGAAGCTTTTAGAAACT |
| | | | ATGTGGAGAAAGAAAGTGGAGATAACACCAAATGTACTG |
| | | | CAGTTCATGATCGACCTCCTTGCGTTGAGATTATACATGA |
| | | | CAACAAACAGAACGGAGAAAGTGATGTGAAGATGCCCCT |
| | | | TCTGGTTTATGTAGCCAGGGAAAAGAGACCTGGTCGTCCT |
| | | | CATCGTTTCAAAGCTGGAGCCCTTAATGCTCTTCTTCGAGT |
| | | | ATCCAGTTTAATGAGCAATGCACCTTACTTATTGGTGTTGG |
| | | | ATTGTGATATGTACTGCCATGATCCAACTTCTGCTCGTCAA |
| | | | TCTATGTGCTTCCATCTTGACACAAACATGGCTTCCTCTCT |
| | | | TGCATATGTGCAATACCCTCAAATTTTCTATAATGTTAGCA |
| | | | AAAATGACATCTATGATGGCCAAGCCAGATCAGCTCATAT |
| | | | GACGAAATGGAAAGGCATGGATGGACTCAGAGGCCCGGT |
| | | | CTTGAATGGAACTGGGTATTATTTGAAGCGAAAAGCATTA |
| | | | TTTGGAAAGCCTAATAACGAAGATGAATACCTCAACAGTC |
| | | | AACCAGAAAAGGCCTTTGGCTCCTCCACAAAATTAATTGC |
| | | | TGCACTAAGAGAGAACTCCAAGCAAAATCTTGCCATAAA |
| | | | GGAATTGACAGAAGATGAGTTGTACCAAGAGGCTAGAAA |
| | | | TTTGGCTACTTGCACATATGAAGCAAACACACTATGGGGC |
| | | | AGTGAGGTAGGATATTCGTATGAGTGCTTGTTGGAGAGTA |
| | | | CATTCACTGGATATATGTTACATTGCAGAGGATGGAAATC |
| | | | TGTGTATCTTTACCCAAAAAGACCATGCTTCTTGGGATGC |
| | | | ACAACGATTGATATGAAGGATGCTACGGTTCAACTAATAA |
| | | | AATGGACCTCCTCATTACTTGGAATTGCCCTGTCGAAGTCT |
| | | | AGCCCTCTAACTTTGGCCATGTCCAGTATGTCAATCCTGCA |
| | | | AAGCATGTGTTACGCGTACATCACATTTACAGGCCTTTTTG |
| | | | CAGCTCCATTGGTTATATATGGTGTTGTCCTTCCAATAAGC |
| | | | CTATTGAAGGGCTTTCCTATTTTCCCTAAGGTATCGGATCC |
| | | | ATGGATTTTGCCATTTGTGTTGATATTTGTATCCTCCCATC |
| | | | TTCAACATCTATATGAGGTCCTGGAAAGTGACAAATCAGC |
| | | | AACACAATGGTGGAATGAGGTGAGAATTTGGATGATGAA |
| | | | ATCAGTGACAGCCTGTTTGTTTGGGTTGACGGAAGCGATA |
| | | | ATGAAGAAGATTGGAGTACAAACTGCAACATTCAGATTA |
| | | | ACAAATAAGGTAGTTGAGAAGGAAAAGATGGATAAATAC |
| | | | GAGAAGGAGAGGTTTGATTTCTCAGGAGCAGCTATGCTTA |
| | | | TGGTTCCTCTTAATATTTTGGTGGTACTAAATATGGTGTCA |
| | | | TTCATTGGTGGACTCATGAGGGTCATAATCAACAACAGTT |
| | | | ATGATCAAATGTTTGCACAACTTTTCCTCTCCTTTTTTGTC |
| | | | CTACTTCTTAGCTACCCTGTTGTTAAGGGATGGTTA |

Summary: This study uncovered numerous surprising details. For example, SOAP6 may serve as a template in the homology-based search for fucosyltransferase decorating quillaic acid in the biosynthetic pathway of QS-21, a potent vaccine adjuvant form Quillaja saponaria (D. J. Marciani, Is fucose the answer to the immunomodulatory paradox of Quillaja saponins? Int Immunopharmacol. 29, 908-913 (2015).). Also, SOAP10, a member of the BAHD superfamily of acyltransferases is involved in triterpenoid saponin biosynthesis.

### Example 21: Production of Triterpenoid Intermediates in Yeast

Objective: To produce of triterpenoid intermediates in a further heterologous system, in the case the yeast Saccharomyces cerevisiae.

Methods: See Materials and Methods above.

Results: To rule out the involvement of additional plant enzymes in the formation of MA-3-GlcA we expressed SOAP5 in Saccharomyces cerevisiae (FIG. 33D). Since yeast do not produce amyrin-type triterpenoids nor UDP-glucuronate, expression of enzymes that generate these precursors was required in order to provide the substrates essential for SOAP5 activity (P. Arendt et al., An endoplasmic reticulum-engineered yeast platform for overproduction of triterpenoids. Metabolic Engineering. 40, 165-175 (2017); T. Oka, Y. Jigami, Reconstruction of de novopathway for synthesis of UDP-glucuronic acid and UDP-xylose from intrinsic UDP-glucose in Saccharomyces cerevisiae. FEBS Journal. 273, 2645-2657 (2006)).

Expression of SOAPs1, 2, 3, and 4 in yeast resulted in formation of medicagenic acid (FIGS. 40A-40B). However, adding SOAP5 was not enough to produce medicagenic acid 3-O-glucuronide (FIGS. 33A-33F). To provide activated sugar for SOAP5 the UDP-glucose 6-dehydrogenase 1 from spinach (SoUGD1, Sp_189830_psca) catalyzing the conversion of UDP-glucose to UDP-glucuronate, was expressed (SEQ ID NO: 74 [gene] and SEQ ID NO: 75 [polypeptide]).

(SEQ ID NO: 74)
ATGGTGAAGATTTGCTGCATTGGGGCTGGTTATGTAGGAGGCCCTACTATGGC
TGTTATAGCACTCAAGTGCCCAAAGATTGAAGTTGTAGTGGTTGATATATCTGTGTCT
CGGATCACTGCATGGAACAGCGAGCAGCTTCCAATCTATGAGCCGGGTCTAGATGAT
GTGGTTAAGGAATGCCGTGGAAGGAACCTTTTCTTCAGCACTGATGTAGAAAAGCAT
GTTGCTGAGGCTGATATTGTTTTTGTCTCTGTGAATACCCCTACCAAAACCACAGGTC
TTGGAGCAGGCAAAGCTGCTGATTTGACCTACTGGGAGAGTGCTGCCCGTATGATTG
CTGATGTTTCAAAGTCTGACAAAATCGTTGTTGAGAAATCAACTGTGCCAGTGAAAA
CTGCTGAGGCAATCGAAAAGATTCTGACGCACAACAGCAAGGGAATCAACTACCAGA
TCCTTTCAAATCCGGAGTTCCTTGCTGAAGGTACTGCTATTCAGGACCTTTTTCACCCT
GACAGGGTTCTCATCGGTGGCCGGGAAACCCCAGCAGGCCTCAAGGCAGTCCAAGCA
TTGAAGGATGTGTATGCTCAATGGGTTCCTGATGAACGGATCTTAACCACCAATCTTT
GGTCTGCTGAGCTCTCAAAGCTTGCTGCCAACGCCTTCTTAGCACAGAGGATTTCATC
TGTCAATGCAATGTCAGCTCTTTGTGAGGCTACTGGAGCAGATGTTACCCAAGTCGCA
TATGCTGTTGGTAAGGACAGTAGGATTGGGCAAAAGTTTTTGAACGCTAGTGTTGGTT
TTGGAGGGTCTTGCTTCCAGAAAGACATTCTGAACTTGGTTTACATTTGTGAGTGCAA
CGGTCTCCCTGAGGTGGCCGAGTATTGGAAACAGGTAATCAAGGTGAATGATTATCA
GAAGAATCGTTTTGTGAATAGGGTTGTGGCCTCCATGTTCAACACTGTATCGAACAAG
AAGATTGCTGTTCTTGGATTTGCATTCAAAAAGGATACAGGGGATACTAGGGAGACA
CCCGCCATAGATGTGTGCAAGGGTTTGTTGGGAGACAAGGCAAGGTTGAGCATCTAC
GATCCACAAGTCACTGAGGATCAGATTCAGCGAGATCTCACCATGAACAAGTTTGAC
TGGGACCACCCAATTCACCTCCAGCCCACAAGTCCCACAACTGTTAAGCAAGTGAGT
GTTGTTTGGGACGCTTATGAGGCCACCAAAGATGCTCATGCTGTGTGTATTCTGACTG
AGTGGGATGAATTTAAGAAACTTGATTACAAAAGGATTTTTGACAACATGCAGAAGC
CAGCTTTTGTGTTTGATGGAAGGAACATTGTGAATGCAGATGAGCTGAGGCAGATTG
GGTTCATTGTGTACTCAATTGGTAAACCTTTGGATTCATGGCTCAAGGACATGCCTGC
TGTGGCTTAA
(SEQ ID NO: 75)
MVKICCIGAGYVGGPTMAVIALKCPKIEVVVVDISVSRITAWNSEQLPIYEPGLDD
VVKECRGRNLFFSTDVEKHVAEADIVFVSVNTPTKTTGLGAGKAADLTYWESAARMIAD
VSKSDKIVVEKSTVPVKTAEAIEKILTHNSKGINYQILSNPEFLAEGTAIQDLFHPDRVLIGG
RETPAGLKAVQALKDVYAQWVPDERILTTNLWSAELSKLAANAFLAQRISSVNAMSALC
EATGADVTQVAYAVGKDSRIGQKFLNASVGFGGSCFQKDILNLVYICECNGLPEVAEYW
KQVIKVNDYQKNRFVNRVVASMFNTVSNKKIAVLGFAFKKDTGDTRETPAIDVCKGLLG
DKARLSIYDPQVTEDQIQRDLTMNKFDWDHPIHLQPTSPTTVKQVSVVWDAYEATKDAH
AVCILTEWDEFKKLDYKRIFDNMQKPAFVFDGRNIVNADELRQIGFIVYSIGKPLDSWLK
DMPAVA

Consequently, only yeast cells expressing SOAP5 together with all other five genes were able to produce MA-3-GlcA (FIGS. 33A-33F; FIGS. 40A-40B). Similar to N. benthamiana assays, SOAP5 was capable of glucuronidation of triterpenoid intermediates (oleanolic acid, gypsogenic acid and bayogenin) in yeast cells (FIGS. 41A-41F).

Summary: Demonstrated that it was possible to produce and glucuronidate triterpenoid intermediates in a system heterologous to the source enzymes.

### Example 22: Examination of Cellulose Synthase Like Enzymes in Additional Plant Species

Objective: To identify and analyze functional Cellulose Synthase Like G (CSLG) enzymes in non-spinach plant species.

Methods: See Materials and Methods above.

Results: Glucuronic acid attached to a saponin aglycone via the hydroxyl at position C-3 is a common feature for many saponins found in plants, especially in the Caryophyllidae, primitive Rosidae and Asteridae (M. Henry, Saponins and Phylogeny: Example of the “Gypsogenin group” Saponins. Phytochem Rev. 4, 89-94 (2005)). To examine if conjugation of glucuronic acid to the triterpenoid backbone by a CSLG enzyme is not limited to spinach and its family, phylogenetic analysis of CSLG proteins was performed in more than 70 plant species including mosses, gymnosperms, and flowering plants. It appeared that spinach SOAP5 is related to CSLG proteins from the Caryophyllales, Malvales, Apiales, and Fabales orders (FIGS. 42A-42E).

Saponin profiling of selected species, as well as published data, demonstrated the presence of glucuronide-oleanane-type saponins in soy (Glycine max), alfalfa (Medicago sativa), Lotus japonicus and licorice (Glycyrrhiza uralensis) as well as in the Caryophyllales species like beetroot (Beta vulgaris) and quinoa (Chenopodium quinoa) (FIGS. 43A-43C).

The closest homologues of the spinach SOAP5 were then cloned from all of the aforementioned species and expressed transiently in N. benthamiana together with SOAP1-4. MA-3-GlcA formation was observed for all tested CSLG enzymes (FIGS. 42A-42E). Table 17 below provides the nucleic acid and amino acid sequences of these SOAP5 homologs.

TABLE 17
SOAP5 Cellulose synthase Like G homologs
(having glucuronic acid transferase activity)

| | SEQ | |
|---|---|---|
| | ID | |
| Name | NO: | Nucleic Acid/Amino Acid Sequence |
| BvCSLG | 94 | MSSLHICKVQTTRAILSRFHILFHSLAILALFYYRFTSFSTTKSGILPWTLLTTAEV |
| polypeptide | | VLGFVWALTQAFRWRPVLRDVAGWDSIKEEQLPGVDVFICTADPIKEPVLEVM |
| from red | | NTVLSAMALDYPAEKLGVYLSDDGGSPLTREAIKEASKFAKVWLPFCSKYGIKT |
| beet_ | | RCPQAFFSSFCDGERLDWNQDFKADELVLKSKYEAFKNYVEKASEDESKCTMA |
| (XM_ | | HDRSPCVEIIHDNKQNGEGEVKMPLLVYVSREKRPNRPHRFKAGALNALLRVS |
| 010673823.2_) | | GVLSNGPYLLVLDCDMYCNDPTSARQSMCFHLDPKLAPSLAFVQYPQIFYNTSK |
| | | NDIYDGQARSAYKTKWQGMDGIRGPVLTGTGYYLKRKALYGQPHNEDEFLIN |
| | | QPEKAFGSSTKFIASVSSNSKQNMALKEMTRDDLLEEAKNLATCAYESNTEWG |
| | | NKIGYSYECLLESTFTGYLLHCKGWISVYLYPKRPCFLGCTTIDMKDAMVQLM |
| | | KWTSGLLGVGISKFSPLTYAFSRMSILQSMCYGYFTFSALFGVSFLIYGIVLPVCL |
| | | LKGVPVFPKVSDPWIGVFVVVFASSLLQHLYEVLSSDDSIKTWWNEIRIWIIKSV |
| | | TASLFGTMDAIMKKIGIQKASFRLTNKVVDKEKLEKYEKGKFDFQGAAVFMVP |
| | | LIILVVLNMVSFVGGLRRAIINKNCDEMFGQLFLSFFLLVLSYPVLEGIVTKVRKG |
| | | RD |
| BvCSLG_ | 95 | CATCACAGCCACATGGGAAACAAAAAGTCTTAACCTCGGCCATCTCGTGTG |
| gene from | | CCTCGTGCACATTCAACATTGATTTTAATTGTGTGCTTTCTCCAAAATGTACC |
| red beet | | ATACTATATCATCTTGTCAGAATTCCAAACTCCAACTATAACCACCATTGAA |
| (XM_ | | GTCATCTACACACAAACACACACACTCTTTCTCCCTAAAAATGTCTTCTCTC |
| 010673823.2) | | CACATTTGCAAAGTCCAAACAACAAGAGCAATACTTAGCCGTTTCCACATA |
| | | CTCTTCCACTCCTTAGCCATCCTTGCTTTATTCTACTACCGTTTTACATCGTTC |
| | | TCTACCACCAAATCAGGCATACTTCCATGGACCTTACTAACCACAGCAGAG |
| | | GTGGTTCTAGGCTTTGTATGGGCGTTAACACAGGCCTTTCGATGGCGGCCTG |
| | | TGTTGCGAGATGTAGCTGGATGGGATTCCATCAAGGAGGAACAACTGCCAG |
| | | GGGTGGACGTGTTCATATGCACAGCTGATCCAATAAAGGAGCCGGTGTTAG |
| | | AGGTGATGAACACGGTGCTTTCGGCGATGGCATTGGATTACCCGGCAGAGA |
| | | AGTTGGGTGTTTATCTTTCGGATGATGGAGGTTCTCCCTTGACTAGGGAGGC |
| | | TATTAAGGAGGCTTCTAAGTTTGCTAAGGTTTGGCTTCCTTTTTGTAGTAAGT |
| | | ATGGTATCAAGACTAGGTGTCCTCAGGCTTTCTTCTCTTCTTTTTGTGATGGG |
| | | GAAAGACTTGATTGGAATCAGGACTTTAAGGCTGATGAATTGGTGCTCAAG |
| | | TCAAAATATGAAGCTTTTAAGAATTATGTGGAGAAAGCAAGTGAAGATGAA |
| | | AGCAAATGCACCATGGCACATGATCGTTCCCCTTGCGTTGAGATTATACATG |
| | | ACAACAAGCAAAATGGAGAAGGCGAAGTGAAAATGCCCCTTTTGGTCTACG |
| | | TATCCAGGGAAAAGAGACCAAATCGTCCTCATCGTTTCAAAGCCGGAGCTC |
| | | TTAATGCTCTTCTCAGAGTATCAGGTGTATTGAGCAACGGGCCTTACTTATT |
| | | GGTGTTGGACTGTGATATGTACTGCAATGATCCAACTTCTGCTCGTCAATCT |
| | | ATGTGCTTTCATCTTGACCCAAAATTGGCTCCTTCACTTGCATTTGTGCAATA |
| | | CCCACAAATTTTCTACAACACCAGTAAAAATGATATCTATGATGGCCAAGC |
| | | TAGATCCGCGTACAAGACAAAATGGCAAGGAATGGATGGTATTAGAGGAC |
| | | CAGTCTTGACAGGAACAGGGTATTACTTGAAGAGGAAAGCATTGTATGGAC |
| | | AACCTCATAACGAAGATGAATTTCTCATTAATCAACCAGAGAAGGCCTTCG |
| | | GCTCCTCCACAAAATTCATTGCGTCAGTTAGTTCAAACTCCAAGCAAAATAT |
| | | GGCCTTGAAGGAAATGACAAGAGACGACTTGTTAGAAGAGGCTAAAAATTT |
| | | GGCTACTTGTGCATATGAATCAAACACTGAATGGGGTAACAAGATTGGATA |
| | | TTCGTATGAGTGTTTGTTGGAGAGTACATTTACCGGATATCTCTTACATTGC |
| | | AAAGGATGGATTTCCGTGTATCTTTACCCAAAAAGACCCTGCTTCTTAGGAT |
| | | GCACGACGATTGACATGAAAGATGCCATGGTTCAACTAATGAAATGGACCT |
| | | CTGGATTACTAGGAGTTGGCATATCAAAGTTTAGCCCTCTAACTTATGCCTT |
| | | TTCGAGGATGTCTATATTACAAAGCATGTGCTACGGTTACTTCACATTTTCA |
| | | GCCCTTTTCGGAGTTTCGTTCTTAATATATGGCATCGTCCTTCCAGTATGCCT |
| | | ATTGAAGGGTGTTCCTGTTTTTCCTAAGGTATCGGATCCATGGATTGGAGTT |
| | | TTCGTGGTAGTATTTGCATCCTCCCTCCTTCAACATTTATACGAGGTTCTCTC |
| | | AAGTGACGATTCCATTAAAACATGGTGGAACGAGATCAGAATTTGGATCAT |
| | | CAAATCGGTAACAGCTTCCTTATTTGGAACAATGGATGCAATAATGAAAAA |
| | | GATCGGCATACAAAAGGCTAGTTTCCGATTAACTAACAAGGTTGTGGACAA |
| | | GGAAAAGCTCGAAAAATATGAGAAGGGCAAGTTTGATTTCCAAGGAGCAG |
| | | CTGTGTTCATGGTTCCTCTTATCATTTTAGTGGTACTAAATATGGTGTCATTT |
| | | GTTGGCGGATTAAGAAGGGCAATAATCAACAAGAATTGTGATGAAATGTTT |
| | | GGGCAACTTTTCCTCTCATTCTTTCTCTTAGTTCTTAGCTACCCCGTTTTAGA |
| | | AGGGATAGTAACAAAAGTAAGAAAAGGACGTGATTGAGATGAATTTGCATT |
| | | GTTTGGTAAAAGATCCAAACTTAGAGAAAGAGATTGCGTAGGAGATCAAAG |
| | | GAAACAATGTGAGAGATTTACAGGCTTCATGAGGCTTAAGACCTCATTAAT |
| | | TTTTGTGACAATTTACAAATTCTGTCTCTATATTTTGGTCAAGACGTATCATT |
| | | TGAAAATTTCCATGGTTAGGTAGTTAGATTTCAATGTTCCACGTTTGTAAAT |
| | | AAGAAGATAAAATAAGGAAATTTGTGATTTTAGCTTTACATTTCTATGAGAT |
| | | AGTCCTTTGTTGTGATGAAAGTTGTGTTCTTAGTGAAAATAATAAAACGTGA |
| | | CATCAAAATTTTGAGTATAT |
| CqCSLG | 96 | MAATHICKVQTKRVIINRIHILFHSLAILALFYYRFSSFSNPHISLFPWVLL |
| Protein | | TIADLVFTFIWAMTQAFRWRPVLHDVSGYESINPRDLPKIDIFICTADPTK |
| from | | EPVLEVMNSVISSMALDYPPEKMAVYLSDDGGSPLTREAIKKAVEFAKV |
| quinoa_ | | WIPFCNMYGIKTRCPDAFFSALGNDERLHRDQDFNAHESLLKSKYEAFK |
| (XM_ | | KYVEKESGDINKCTVVHDREPCIEIIHDSKQDGEAEVKMPLVVYVAREK |
| 021866098.1)_ | | RPGHPHRFKAGALNALLRVSGLLSNAPYLLVLDCDMYCHDPTSARQSM |
| | | CFHLDPNMSPSLAFVQYPQIFYNTSKNDIYDGQARSAHTTKWQGMDGL |
| | | RGPVLNGTGYYLKKKAIYGRPHNEDEYLINEPEKAFGSSTKFIASLKENS |
| | | NQDLVLKEFTNDLLQEARNLATCTYEANSLWGVEVGFSYDCLLESSYT |
| | | GYLLHCKGWRSVYLYPKRPCFLGCTTIDMKDAIVQLIKWTSGLLGVAM |
| | | SKFSPLTYAMSRMSILQSMCYAYITCSGLLAVPLFIYGVVLPFCLLKGVP |
| | | VFPKVSDPWMLGFVFVFVSSHVQHLFEVLASDHSVQQWWNEVRIWIM |
| | | KAITACLFGSTEAIMKKIGIQKTTFRLTNKVVEKEKLDKYEKGKFDFSGA |
| | | AMLMVPLIILTILNLVSFVGGLVRVINHNNYDDMFGQLFLSFYLLLLSYP |
| | | TFEGIVTKVTDKLRKKE |
| CqCSLG | 97 | AAAAGTGAAACTGGTGACTTAGTCTTGTGTCACCCGGGTTCCTCGAGCACAT |
| Gene from | | ATTCTTCAAGATTTTGGTTTTTTGTACGGAGTATTTATATACACAAAAATTA |
| quinoa_ | | GGAACCAAATAGGAAGACTCATATCATTTCAAAATGGCGGCAACACACATT |
| (XM_ | | TGCAAAGTCCAAACCAAAAGAGTCATTATCAACCGTATTCATATCCTCTTTC |
| 021866098.1) | | ACTCTTTAGCCATTCTTGCTCTCTTCTACTACCGTTTCTCGTCTTTCTCCAACC |
| | | CTCATATCTCCCTTTTTCCATGGGTATTATTGACTATCGCCGACCTCGTTTTC |
| | | ACCTTCATTTGGGCCATGACTCAGGCCTTCCGTTGGCGCCCCGTCTTGCACG |
| | | ATGTGTCTGGCTATGAGTCCATCAATCCACGCGATCTTCCAAAGATCGATAT |
| | | TTTTATATGCACCGCTGATCCCACCAAGGAGCCTGTGTTGGAAGTGATGAAC |
| | | TCGGTGATATCATCCATGGCGCTCGATTATCCGCCTGAAAAAATGGCGGTGT |
| | | ATTTGTCGGATGATGGTGGTTCTCCTTTGACTAGAGAGGCTATTAAGAAGGC |
| | | TGTTGAATTTGCTAAGGTTTGGATTCCTTTTTGTAATATGTATGGTATTAAGA |
| | | CTAGGTGTCCTGATGCTTTCTTCTCCGCTTTGGGTAATGATGAAAGACTTCA |
| | | TCGTGATCAAGACTTTAACGCTCATGAATCACTCCTCAAGTCGAAATACGA |
| | | AGCTTTTAAGAAATATGTGGAGAAAGAAAGCGGTGATATTAATAAATGCAC |
| | | CGTTGTGCATGATCGTGAACCTTGCATTGAGATTATACATGACAGTAAACA |
| | | GGATGGAGAAGCTGAAGTGAAAATGCCCCTTGTAGTTTATGTAGCCAGGGA |
| | | AAAGAGACCAGGTCATCCTCATCGTTTCAAAGCTGGAGCCCTTAACGCTCTT |
| | | CTCCGAGTATCAGGACTATTGAGCAATGCGCCTTACTTATTGGTGTTAGACT |
| | | GTGATATGTACTGTCATGATCCAACCTCTGCTCGTCAATCTATGTGCTTCCAT |
| | | CTTGACCCGAACATGTCTCCCTCTCTTGCCTTTGTTCAATACCCTCAAATTTT |
| | | CTACAACACTAGTAAAAATGATATCTATGATGGTCAAGCCAGATCAGCTCA |
| | | TACGACGAAATGGCAAGGCATGGATGGACTCAGAGGACCGGTCTTGAATGG |
| | | AACTGGGTATTATCTGAAGAAGAAGGCGATATATGGAAGGCCCCATAATGA |
| | | AGATGAATACCTCATCAATGAACCAGAAAAGGCTTTTGGTTCTTCCACAAA |
| | | ATTCATTGCTTCACTTAAAGAAAACTCGAACCAGGATCTTGTCTTGAAGGAA |
| | | TTCACAAACGATTTGTTACAAGAGGCTAGAAATTTGGCTACTTGCACTTATG |
| | | AAGCAAACTCGCTATGGGGTGTTGAGGTAGGGTTTTCGTATGATTGCCTGTT |
| | | GGAGAGTTCATACACTGGATATCTCTTACATTGTAAAGGATGGAGATCTGT |
| | | GTATCTTTATCCCAAAAGACCGTGCTTCTTGGGATGCACGACAATTGACATG |
| | | AAGGATGCTATTGTTCAATTAATAAAATGGACTTCCGGATTACTTGGAGTTG |
| | | CCATGTCAAAGTTTAGCCCTCTTACTTATGCCATGTCCAGAATGTCTATATT |
| | | GCAAAGCATGTGTTACGCGTACATCACGTGTTCAGGTCTTCTAGCAGTTCCA |
| | | CTCTTTATATATGGTGTTGTTCTACCATTCTGCCTACTTAAGGGCGTTCCTGT |
| | | TTTTCCTAAGGTATCGGATCCATGGATGTTGGGTTTCGTGTTTGTATTTGTAT |
| | | CCTCCCATGTTCAACATCTATTCGAAGTGCTAGCAAGTGATCATTCAGTGCA |
| | | ACAGTGGTGGAATGAGGTGAGAATCTGGATCATGAAAGCGATAACAGCCTG |
| | | CTTGTTTGGATCAACTGAAGCAATAATGAAGAAGATTGGGATACAGAAAAC |
| | | AACATTCAGATTAACAAATAAGGTTGTGGAGAAAGAGAAGTTGGATAAATA |
| | | CGAGAAGGGAAAGTTCGATTTCTCAGGAGCAGCAATGCTAATGGTTCCTCT |
| | | CATCATTTTGACTATACTAAATTTGGTGTCGTTCGTTGGGGGACTTGTAAGG |
| | | GTGATCAACCACAACAACTATGATGATATGTTCGGGCAACTTTTCCTGTCAT |
| | | TTTATCTCCTACTTCTTAGCTACCCTACTTTCGAAGGGATTGTTACAAAAGTT |
| | | ACAGACAAACTTAGAAAGAAAGAATAAGGAGTGATTGAGTAACTGCCTAG |
| | | TACAGTTTTCACTTCACTTCTCTAGATTAGTCCTTGTTTTTGTTTATGTTTATT |
| | | AAGATCAGCAACACTTGTAGACGGTTGCAATAATGAGTTCAATACCGTTTG |
| | | TTCTGTCCCTCTTGCAAGAACAAGTATATAAATACTTTTCATTAGCCGGTTG |
| | | CATTGTTGGATTCATATAGATGAATATTTCAAATATTTCATCTTTTTGAACTT |
| | | ACACACTAATGATTTATATCTGGAATTTTGAAA |
| MsCSLG | 98 | MATFTFHKETVQPLLPLRRAYIIFHFTCVLFLFYYRISNLFISYPWFLMTIAEIILSF |
| Protein | | LWFFNQAFRWRLVNRSVMTEKLPPEEKLPGLDIFVCTIDPEKEPTVDVMNTVIS |
| from | | AIAMDYPSNKLSIYLSDDGGSPITLFGIKEAFEFAKVWVPFCKKYDVKSRCPKFF |
| Alfalfa_ | | FTALGENERLHRPREFEEVRDQIKKRLNRVDPNSSQVENSKEHMPTKAKYEKM |
| (MSAD_ | | QKNIEKFGSNLKNLCMVTDRPSRIEIINDQKEMPLVVYVSREKRPSVPHRFKGG |
| 299835) | | ALNTLLRVSGLISNGPYVLVVDCDMNCNDASSAKQSMCFFLDPETSKDVAFVQ |
| | | FPQMFHNLSKKDIYDSQTRTAFTTKWKGMDGLRGPGLTGSGNYISRSALLFGSP |
| | | NQKGDYLLDALYNFGKSNMYVESLKALRGQQTKKQNISRDVILQEACEVASCS |
| | | YERNTNWGNEVGFSYAIKLESTVTGYLLHCRGWRSTYLYPKRPCFLGCAPTDM |
| | | KEGLIQPIKWSSELLLLAISKYSPFTYGLSRLPTIHCLTFCYLVSTTQFATAYILYG |
| | | FVPQICFLKGIPVYPKVTDPWFIVFTVLYLSSQIHHYIEVISTGGSSMIWWNEQRS |
| | | GIVKSIGCVFAIIETAKKKFGLNKAKFTLSDKAIDKDKLKKYEQGKFNFDGAAL |
| | | LMAPVIVLLTINIVCFFGGLWRLLNVRDFDEMFGQLFLIIYILALSHPIVEGIISMK |
| | | RKSG |
| MsCSLG | 99 | ATGGCAACCTTCACATTTCACAAAGAAACAGTTCAACCATTGTTACCTCTAA |
| Gene | | GAAGAGCTTACATAATCTTCCACTTCACATGTGTCTTGTTTCTCTTTTACTAC |
| from | | CGTATCAGCAATTTGTTTATTTCATATCCATGGTTTCTAATGACAATAGCTG |
| Alfalfa_ | | AGATTATTCTATCATTTCTATGGTTTTTCAACCAAGCATTCCGTTGGAGGCTG |
| (MSAD_ | | GTGAATCGTTCAGTTATGACCGAGAAATTACCGCCGGAGGAGAAGTTGCCG |
| 299835) | | GGACTCGACATATTTGTGTGTACCATTGATCCTGAAAAAGAACCAACGGTT |
| | | GATGTTATGAACACTGTTATTTCTGCTATTGCAATGGATTACCCTTCTAATA |
| | | AACTTTCTATTTATCTTTCTGATGATGGAGGTTCTCCTATTACTCTTTTTGGG |
| | | ATCAAAGAGGCTTTTGAATTTGCTAAAGTTTGGGTTCCTTTTTGTAAAAAAT |
| | | ATGATGTTAAGTCAAGGTGTCCTAAGTTTTTCTTCACTGCTTTGGGTGAGAA |
| | | TGAACGACTTCATCGACCTCGTGAATTTGAAGAAGTGAGGGACCAGATTAA |
| | | GAAGAGATTAAATAGAGTGGATCCTAACTCATCACAAGTTGAAAACTCCAA |
| | | GGAACATATGCCCACCAAAGCCAAATACGAGAAAATGCAGAAAAATATTG |
| | | AGAAATTCGGAAGCAACCTAAAGAATCTTTGTATGGTGACCGATAGACCTT |
| | | CTCGGATCGAGATCATTAATGACCAAAAAGAAATGCCACTAGTTGTTTATG |
| | | TATCTCGTGAAAAAAGACCATCTGTTCCTCACAGATTCAAAGGAGGAGCTC |
| | | TCAATACATTGCTTAGGGTGTCAGGGCTAATCAGCAATGGACCTTATGTACT |
| | | TGTCGTAGATTGTGATATGAATTGTAATGATGCATCATCAGCCAAACAATCC |
| | | ATGTGCTTTTTTCTTGATCCTGAAACCTCTAAAGATGTTGCTTTTGTTCAATT |
| | | CCCTCAAATGTTTCACAACCTTAGCAAGAAAGACATATATGATAGTCAGAC |
| | | TAGGACTGCTTTTACGACAAAGTGGAAGGGAATGGATGGATTAAGAGGTCC |
| | | AGGTCTAACTGGCAGTGGAAATTATATAAGTAGAAGTGCATTACTCTTTGG |
| | | AAGTCCAAACCAAAAAGGGGACTATCTACTTGATGCTCTATACAACTTTGG |
| | | CAAGTCTAACATGTATGTAGAATCACTAAAAGCGTTACGTGGTCAACAAAC |
| | | TAAGAAGCAGAATATTTCAAGAGATGTAATTTTACAAGAAGCATGTGAAGT |
| | | GGCTTCTTGTTCCTATGAGAGAAACACAAATTGGGGTAATGAGGTGGGATT |
| | | CTCGTATGCTATAAAACTTGAGAGTACCGTTACTGGCTATCTCCTCCATTGT |
| | | AGAGGATGGAGATCAACTTATCTTTACCCTAAAAGACCATGTTTCTTAGGAT |
| | | GTGCTCCAACTGACATGAAAGAGGGATTGATTCAACCGATAAAGTGGTCAT |
| | | CTGAACTTTTGTTGCTTGCAATCTCTAAATATAGCCCATTCACTTATGGCCTT |
| | | TCAAGATTGCCCACTATTCATTGTTTAACTTTTTGTTACTTGGTAAGCACAAC |
| | | CCAATTTGCAACAGCCTACATCTTATATGGATTCGTTCCTCAGATTTGCTTCT |
| | | TGAAGGGAATACCTGTATATCCAAAGGTTACAGATCCTTGGTTTATAGTGTT |
| | | TACAGTATTGTATCTATCCAGTCAAATTCATCATTATATTGAGGTAATTTCA |
| | | ACTGGTGGCTCCTCGATGATTTGGTGGAATGAACAAAGAAGTGGGATTGTA |
| | | AAATCAATTGGGTGCGTTTTCGCAATTATAGAAACAGCGAAAAAAAAGTTT |
| | | GGGTTGAACAAGGCAAAATTCACTTTATCGGACAAAGCAATTGACAAAGAT |
| | | AAGCTAAAGAAATATGAGCAGGGTAAGTTTAATTTTGATGGTGCAGCATTG |
| | | CTCATGGCACCAGTGATTGTGTTACTCACAATAAATATTGTTTGCTTCTTTGG |
| | | TGGTTTATGGAGACTACTCAATGTGAGGGATTTTGATGAAATGTTTGGTCAA |
| | | CTTTTCCTCATTATCTATATACTTGCTCTAAGTCATCCTATTGTGGAGGGGAT |
| | | TATATCTATGAAGCGGAAGAGTGGGTAG |
| GmCSLG | 100 | MATFHTETVQSGLALSRLHILFHSVALLFLYYYRISHILLEPSFVWIFMTI |
| Protein | | AELIFGELWLFKQAFRWRPVSRAVMPEKLPSDGKLPALDIFVCTVDPEK |
| from | | EPTVQVMDTVISAIAMDYPSNKLAVYLSDDGGCPVTLYGIREASRFAKE |
| Soybean_ | | WVPFCRKYGINSRCPKAFFSPMGEDERELLLLRNHEFLAEQEQLKAKYN |
| (NM_ | | IMQKNIDEFGRDPKNRSIVFDRPARIEIINEQSEIPLVVYVSRERRPNVPHT |
| 001365113.1_ | | YKGGALNTLLRVSGLFSNGPYVLVVDCDMYCNDPSSAKQAMCFFLDPE |
| soja) | | TSKDIAFVQFPQMFHNLSMKDIYDSQHRHAFTTMWQGMDGLRGPGLS |
| | | GSGNYLSRSALIFPSPYEKDGYEHNAQNKFGNSTMYIESLKAIQGQQTY |
| | | KTSISRNVILQEAQAVASCSYEIDTNWGNEVGFSYVILLESTVTGYLLHC |
| | | RGWRSTYLYPKRPCFLGCAPTDFMEGMLQLVKWSSELFLLGISKYSPFT |
| | | YGISRIPILHNFTFCYFTSTCQYIVALIVYGIIPQVCFLKGTPVFPKVTEPW |
| | | FVVFAILYVSSQSQHLIEVLYGGGSLGTWWDEQRIWIVKSIVGGIFGSILA |
| | | IKKRFGLNKAKFILSNKVVAKEKFEKYEQGKFEFEDAALFMSPLVGLLIV |
| | | NILCFFGGLWRLFNVKDFEKMSGQLFLLGYLAALSYPIFEGIITMKSKVQ |
| GmCSLG | 101 | TATATGCATGTTGACCGGTAAACATGGCGACCTTCCACACAGAAACC |
| gene from | | GTGCAATCAGGGTTGGCCTTGAGCAGACTCCACATCCTATTCCACTC |
| Soybean_ | | GGTGGCACTCTTGTTTCTCTATTACTACCGCATAAGCCACATCTTACT |
| (NM_ | | GGAACCAAGCTTTGTATGGATTTTCATGACCATAGCGGAGCTTATCT |
| 001365113.1_ | | TCGGCGAGCTCTGGCTCTTCAAACAGGCGTTCCGGTGGCGGCCCGTG |
| soja) | | TCGAGGGCCGTCATGCCGGAGAAGCTGCCGAGCGACGGCAAGCTTC |
| | | CGGCGCTCGACATCTTCGTCTGCACGGTTGACCCCGAAAAGGAGCCG |
| | | ACGGTGCAGGTGATGGACACCGTCATCTCCGCCATTGCCATGGACTA |
| | | CCCCTCCAACAAGCTCGCCGTGTACCTTTCCGACGATGGCGGGTGTC |
| | | CGGTGACTCTGTATGGGATCAGAGAGGCTTCTCGGTTCGCAAAGGAG |
| | | TGGGTTCCGTTCTGCAGAAAGTATGGGATCAATTCACGGTGCCCCAA |
| | | GGCCTTCTTCTCTCCCATGGGGGAGGATGAACGTGAACTGCTTCTTCT |
| | | TCGCAACCATGAATTCTTGGCAGAGCAAGAACAACTCAAGGCTAAA |
| | | TACAATATAATGCAAAAAAATATTGACGAATTTGGAAGAGACCCTA |
| | | AAAATCGTTCCATTGTGTTTGATAGACCAGCTCGCATTGAGATTATA |
| | | AATGAGCAATCCGAAATACCACTGGTTGTTTATGTGTCTCGTGAAAG |
| | | AAGGCCAAATGTTCCTCATACATACAAAGGGGGAGCCCTCAACACA |
| | | TTGCTCAGAGTCTCAGGGCTATTCAGTAACGGGCCCTATGTACTTGT |
| | | AGTTGATTGTGATATGTATTGCAATGATCCATCATCAGCTAAACAAG |
| | | CCATGTGCTTTTTTCTTGATCCTGAAACCTCCAAAGATATTGCTTTTG |
| | | TCCAATTCCCTCAAATGTTTCACAACCTTAGCATGAAAGACATCTAC |
| | | GATAGTCAACATAGGCATGCTTITACAACAATGTGGCAAGGAATGG |
| | | ATGGACTAAGAGGTCCAGGTCTTTCTGGTAGTGGCAATTACTTAAGT |
| | | AGAAGTGCATTAATCTTTCCAAGCCCATATGAAAAAGACGGCTATGA |
| | | ACATAATGCCCAAAACAAATTTGGCAACTCTACCATGTACATTGAAT |
| | | CATTAAAGGCCATTCAAGGACAACAAACTTATAAAACGAGCATTTCA |
| | | AGAAATGTGATTTTACAGGAAGCACAAGCAGTGGCCTCTTGTTCCTA |
| | | TGAAATAGACACAAATTGGGGTAATGAGGTAGGATTCTCATATGTTA |
| | | TATTACTGGAGAGTACAGTTACTGGCTATCTTCTTCACTGTAGAGGA |
| | | TGGAGATCAACTTACCTTTACCCCAAAAGACCTTGTTTCTTGGGATGT |
| | | GCCCCCACTGACTTCATGGAAGGCATGCTTCAGTTGGTGAAATGGAG |
| | | TTCTGAACTTTTCTTGCTAGGAATATCCAAATACAGCCCTTTCACTTA |
| | | TGGGATTTCAAGAATTCCTATTCTGCACAACTTTACCTTTTGCTACTT |
| | | CACATCTACATGTCAATATATTGTTGCCTTAATAGTATATGGCATCAT |
| | | TCCTCAAGTATGCTTCTTGAAAGGAACTCCTGTGTTTCCTAAGGTTAC |
| | | AGAACCATGGTTTGTAGTTTTTGCAATATTATATGTATCCTCTCAAAG |
| | | TCAACATTTGATTGAAGTCCTTTATGGTGGTGGCTCTTTGGGAACATG |
| | | GTGGGATGAACAAAGAATATGGATTGTAAAGTCAATTGTTGGAGGC |
| | | ATATTTGGATCTATACTAGCAATCAAGAAACGTTTTGGGTTAAACAA |
| | | AGCAAAATTCATTITATCAAATAAAGTTGTTGCCAAAGAGAAGTTTG |
| | | AGAAATATGAACAAGGTAAGTTCGAGTTCGAAGATGCAGCTTTGTTC |
| | | ATGTCTCCATTGGTTGGATTACTCATAGTGAATATTCTTTGCTTCTTT |
| | | GGTGGTTTATGGAGACTATTTAATGTGAAAGATTTTGAAAAGATGTC |
| | | TGGCCAACTTTTTCTACTTGGCTATCTGGCGGCGCTCAGTTATCCCAT |
| | | TTTTGAGGGGATAATAACCATGAAAAGCAAGGTGCAATAGTAGTTTG |
| | | TCAATGATTAGGCTAATTTAGGTATTTGAACTTTGTTCACAAATAATT |
| | | TGCTTCATATGAAAATCTAAAGTGCATGCTAAATGTTTGTATCTTAAT |
| | | ATGTAATTAGCGTGCTTTTATTTCATGCATGAGAATATGGCTATCGAT |
| | | TTTAATTAGGAGCAAAATGTATGTTCTTACTCCATTTTTAATGCAATT |
| | | TCTTTATTTTCTTGCCAATTAAA |
| LjCSLG | 104 | MANFTLHTETVQAWLPLSRLHILIHSVFVILLLYYRTTRLIHAPTAPWIL |
| Protein | | MTVAEALLAVLWLFNQAFRWRPVSRSVKTEKLPRDENLPGLDIFVCTID |
| from Lotus | | PEKEPTAGVMDTVVSAVAMDYPPDKLSVYLSDDGGCAVTEYGIREACE |
| japonicus_ | | FAKVWVPFCRKYGIKSRCPKVFFSPMGEDEEILRTDEFRAEQEKIKAQYE |
| (Lj3g3v1981230.1)_ | | TMQKNIEKFGSDPKNCRIVTDRPSKIEVINEQSEIPRVVYVSRERRPSLPH |
| | | KFKGGALNTLVRVSGLISNGPYVLAVDCDMYCNDPSSAKQAMCFFLDP |
| | | ETSKYIAFVQFPQMFHNLSKKDIYDNQSRTAFKAMWQGMDGLSGPGLS |
| | | GSGNYLSRSALLFGSPNQKGDYLLDAQNYFGESPLYIESLKAIRGQQTTK |
| | | KNISRDESLLEAKVVASASYETNTEWGSEVGFSYGILLESTITGYLLHCR |
| | | GWKSAYLYPKTPCFLGCAPTDIKEGMLQLVKWLSELCLFAVSKYSPFTY |
| | | GFSRLPIMPTFTYCFLAASSLYAIVFILYGIVPQVCFLKGIPVFPKATDPWF |
| | | AVFAVLYVATQIQHLIEVLSGNGSVSMWWDEQRIWILKSVTSVFAMIEG |
| | | IKKWLGLNKKKFNLSNKAVDKEKVKKYEQGRFDFQGAALYMSPMVVL |
| | | LLVNIVCFFGGLWRLFKEKDFADMFGQLFLLSYVMALSYPILEGIVTMK |
| | | MKSG |
| LjCSLG | 105 | ATGGCCAATTTCACTCTCCACACAGAAACCGTTCAAGCATGGCTCCC |
| Gene from | | TCTAAGCAGACTCCACATTCTTATACACTCAGTGTTCGTCATCCTTCT |
| Lotus | | CCTCTACTACCGCACAACGCGTCTCATCCACGCGCCGACCGCGCCGT |
| japonicus_ | | GGATCCTGATGACCGTTGCGGAGGCTCTCCTCGCCGTGCTTTGGCTCT |
| (Lj3g3v1981230.1)_ | | TCAACCAGGCCTTCCGGTGGCGACCGGTGAGCCGCTCCGTGAAGACA |
| | | GAGAAGCTGCCGCGCGACGAGAATCTCCCCGGGCTGGACATATTTGT |
| | | GTGCACGATTGATCCTGAGAAGGAGCCAACGGCAGGGGTGATGGAC |
| | | ACGGTTGTTTCCGCCGTGGCGATGGATTACCCGCCGGATAAGCTATC |
| | | CGTGTATCTTTCTGATGATGGTGGTTGCGCCGTGACGGAGTATGGGA |
| | | TTAGAGAGGCTTGTGAGTTTGCCAAGGTGTGGGTTCCTTTTTGTAGA |
| | | AAGTATGGGATCAAGTCGAGGTGTCCAAAAGTTTTCTTCTCTCCGAT |
| | | GGGGGAAGATGAAGAGATTCTAAGGACAGATGAGTTCAGAGCAGAG |
| | | CAAGAGAAGATCAAGGCCCAATACGAGACTATGCAGAAAAACATCG |
| | | AGAAATTTGGTTCAGACCCCAAAAATTGTCGTATTGTGACTGACAGA |
| | | CCCTCTAAGATCGAGGTTATAAATGAGCAATCAGAAATCCCACGTGT |
| | | TGTGTACGTCTCTCGTGAAAGAAGGCCATCACTTCCTCACAAGTTCA |
| | | AAGGAGGAGCTCTCAACACATTGGTCAGAGTGTCAGGTCTAATCAGC |
| | | AATGGACCTTATGTGCTTGCAGTGGATTGTGATATGTATTGCAATGA |
| | | TCCATCCTCTGCCAAGCAAGCAATGTGCTTCTTCCTTGATCCAGAAA |
| | | CATCTAAATACATTGCATTTGTCCAATTCCCTCAAATGTTTCACAACC |
| | | TTAGTAAGAAAGACATCTATGATAATCAATCTAGGACTGCTTTTAAG |
| | | GCAATGTGGCAAGGCATGGATGGACTCAGTGGTCCAGGTCTTTCTGG |
| | | CAGTGGTAACTACTTGAGTAGAAGTGCATTGCTATTTGGAAGTCCAA |
| | | ACCAAAAAGGTGACTATCTGCTTGATGCTCAAAACTACTTTGGCGAG |
| | | TCTCCCTTGTACATTGAATCATTGAAGGCCATCCGTGGACAACAAAC |
| | | TACCAAAAAGAATATCTCAAGAGACGAAAGTTTACTAGAAGCTAAA |
| | | GTGGTGGCCTCTGCTTCCTACGAGACAAACACAGAATGGGGCTCAGA |
| | | GGTTGGATTCTCATATGGCATCTTACTGGAGAGTACTATTACTGGTTA |
| | | CCTTTTGCACTGCAGAGGATGGAAATCAGCTTATCTTTACCCAAAAA |
| | | CACCATGTTTCTTAGGGTGTGCCCCCACTGACATTAAAGAAGGCATG |
| | | CTTCAGTTGGTGAAGTGGTTGTCTGAGCTTTGCTTGTTTGCTGTCTCT |
| | | AAGTACAGCCCTTTTACATATGGGTTTTCAAGATTGCCCATTATGCCT |
| | | ACCTTCACTTATTGTTTCCTGGCAGCTTCATCCCTATATGCTATTGTCT |
| | | TCATCCTTTATGGCATTGTACCTCAAGTGTGCTTCTTGAAAGGAATCC |
| | | CTGTGTTTCCAAAGGCCACAGACCCTTGGTTTGCAGTGTTTGCAGTAT |
| | | TGTATGTAGCCACCCAGATTCAACATTTGATTGAAGTCCTTTCTGGCA |
| | | ATGGCTCGGTCTCGATGTGGTGGGATGAACAAAGAATTTGGATTCTG |
| | | AAGTCAGTTACTAGCGTATTTGCAATGATAGAGGGAATCAAGAAAT |
| | | GGTTAGGATTGAACAAGAAAAAATTCAACCTGTCAAACAAAGCGGT |
| | | TGACAAGGAGAAGGTCAAGAAATATGAGCAAGGTAGGTTTGATTTC |
| | | CAAGGAGCAGCTCTGTACATGTCTCCAATGGTTGTGTTGCTCCTAGT |
| | | GAACATTGTTTGCTTCTTTGGCGGTTTATGGAGACTGTTTAAGGAGA |
| | | AAGATTTTGCAGATATGTTTGGTCAACTTTTCCTACTCAGCTATGTGA |
| | | TGGCTCTCAGTTATCCCATTCTTGAGGGGATAGTAACTATGAAAATG |
| | | AAGAGTGGGTAG |

Silencing the beetroot CSLG (BvSOAP5), as in the case of spinach SOAP5, resulted in decreased levels of saponins and elevated accumulation of oleanolic acid (FIGS. 44A-44C). Similarly, suppressed expression of CLSG in alfalfa hairyroot resulted in decreased accumulation of saponins with attached glucuronic acid (FIGS. 45A and 45B).

Summary: These results demonstrate heterologous functionality of enzymes within the saponin biosynthetic pathway.

### Example 23: Heterologous Production of Glycyrrhizin Using Cellulose Synthase Like G from Licorice

Objective: The discovery of CSLG glucuronosyltransferase activity fills a large knowledge gap in the biosynthetic pathways of numerous glucuronide-type triterpenoid saponins. One such pathway only partly deciphered to date generates glycyrrhizin; a triterpenoid saponin produced in licorice consisting of glycyrrhetinic acid decorated with two glucuronic acid moieties at the C-3 position. Glycyrrhizin (i.e. GL) and glycyrrhetinic acid monoglucuronide (i.e. GAMG), the single glucuronidated derivative, are important natural products long used in traditional Chinese and Japanese medicine and nowadays in the food, cosmetics and pharmaceutical industry. These molecules are low-calorie sweeteners with no glycemic index; GAMG is 941-fold sweeter than sucrose and 5-fold more than that of GL (K. Mizutani, T. Kuramoto, Y. Tamura, N. Ohtake, S. Doi, M. Nakaura, O. Tanaka, Sweetness of glycyrrhetic acid 3-O-beta-D-monoglucuronide and the related glycosides. Biosci Biotechnol Biochem. 58, 554-5 (1994)). Commercial production of GL and GAMG depends on the currently limited availability of wild G. uralensis and inefficient industrial processing. Despite its importance, sustainable production of GL and GAMG in heterologous systems is currently impossible since the enzyme catalyzing attachment of the first glucuronic acid to the aglycone was not identified (Y. Nomura et al., Functional specialization of UDP-glycosyltransferase 73P12 in licorice to produce a sweet triterpenoid saponin, glycyrrhizin. Plant J. doi: 10.1111/tpj.14409 (2019)). The goal here was to produce glycyrrhizin in a heterologous system

Methods: See Materials and Methods above.

Results: Some of the saponins, especially those produced by plants from Fabales, are of high importance due to their unique properties. Glycyrrhizin, triterpenoid saponin consisting of glycyrrhetinic acid decorated with two glucuronic acid moieties at position C-3 is produced by G. uralensis and widely used as calorie-free sweetener and a drug in many Asian countries. Four out of five enzymes involved in glycyrrhizin production were already characterized (bAS, CYP88D6 and CYP72A154, UGT73P12) but the enzyme responsible for attaching the first glucuronic acid to the aglycone was missing (Y. Nomura et al., Functional specialization of UDP-glycosyltransferase 73P12 in licorice to produce a sweet triterpenoid saponin, glycyrrhizin. Plant J. doi: 10.1111/tpj.14409 (2019).). The question asked here was if cellulose synthase like G from licorice (GuCSL) was able to perform this reaction.

The cellulose synthase like G gene from G. uralensis was identified and used for expression in a heterologous system. The nucleotide and amino acid sequences of the expressed genes and the encoded polypeptides is provided below in Table 18.

TABLE 18
Components of the triterpenoid Glycyrrhizin
Biosynthetic Pathway in G. uraliensis

| | | SEQ | |
|---|---|---|---|
| | Enzyme | ID | |
| Name | Activity | NO: | Nucleotide Sequence/Amino Acid Sequence |
| GuCYP88D6_ | β-amyrin | 76 | ATGGAAGTACATTGGGTTTGCATGTCCGCTGCCACTTTGTT |
| GLYUR000561S00023451.1 | 11-oxidase | | GGTATGCTACATTTTTGGAAGCAAGTTTGTGAGGAATTTGA |
| | | | ATGGGTGGTATTATGATGTAAAACTAAGAAGGAAAGAACA |
| | | | CCCACTACCCCCAGGTGACATGGGATGGCCTCTTATCGGCG |
| | | | ATCTATTGTCCTTCATCAAAGATTTCTCATCGGGTCACCCTG |
| | | | ATTCATTCATCAACAACCTTGTTCTCAAATATGGACGAAGT |
| | | | GGTATCTACAAGACTCACTTGTTTGGGAATCCAAGCATCAT |
| | | | TGTTTGYGAGCCTCAGATGTGTAGGCGAGTTCTCACTGATG |
| | | | ATGTGAACTTTAAGCTTGGTTATCCAAAATCTATCAAAGAG |
| | | | TTGGCACGATGTAGACCCATGATTGATGTCT |
| | | | CTAATGCGGAACATAGGCTTTTTCGACGCCTCATTACTTCC |
| | | | CCAATCGTGGGTCACAAGGCGCTAGCAATGTACCTAGAGC |
| | | | GTCTTGAGGAAATTGTGATCAATTCGTTGGAAGAATTGTCC |
| | | | AGCATGAAGCACCCCGTTGAGCTCTTGAAAGAGATGAAGA |
| | | | AGGTTTCCTTTAAAGCCATTGTCCACGTYTTCATGGGCTCTT |
| | | | CCAATCAGGACATCATTAAAAAAATTGGAAGTTCGTTTACT |
| | | | GATTTGTACAATGGCATGTTCTCTATCCCCATTAACGTACCT |
| | | | GGTTTTACATTCCACAAAGCACTCGAGGCACGTAAGAAGCT |
| | | | AGCCAAAATAGTTCAACCCGTTGTGGATGAAAGGCGGTTG |
| | | | ATGATAGAAAATGGTCCACAAGAAGGGAGCCA |
| | | | AAGAAAAGATCTTATTGATATTCTTTTGGAAGTCAAAGATG |
| | | | AGAATGGACGAAAATTGGAGGACGAGGATATTAGCGATTT |
| | | | ATTAATAGGGCTTTTGTTTGCTGGCCATGAAAGTACAGCAA |
| | | | CCAGTTTAATGTGGTCAATTACATATCTTACACAGCATCCC |
| | | | CATATCTTGAAAAAGGCTAAGGAAGAGCAGGAAGAAATAA |
| | | | CGAGGACAAGATTTTCCTCGCAGAAACAATTAAGTCTTAAG |
| | | | GAAATTAAGCAAATGGTTTATCTTTCTCAGGTAATTGATGA |
| | | | AACTTTACGATGTGCCAATATTGCCTTTGCAACTTTTCGAG |
| | | | AGGCAACTGCTGATGTGAACATCAATGGTTATATCATACCA |
| | | | AAGGGATGGAGAGTGCTAATTTGGGCAAGAGCC |
| | | | ATTCATATGGATTCTGAATATTACCCAAATCCAGAAGAATT |
| | | | TAATCCATCGAGATGGGATGATTACAATGCCAAAGCAGGA |
| | | | ACCTTCCTTCCTTTTGGAGCAGGAAGTAGACTTTGTCCTGG |
| | | | AGCCGACTTGGCGAAACTTGAAATTTCCATATTTCTTCATT |
| | | | ATTTCCTCCTTAATTACAGGTTGGAGAGAATAAATCCAGAA |
| | | | TGTCACGTTACCAGCTTACCAGTATCTAARCCCACAGACAA |
| | | | TTGTCTCGCTAAGGTGATAAAGGTCTCATGTGCTTAG |
| GuCYP88D6_ | β-amyrin | 77 | MEVHWVCMSAATLLVCYIFGSKFVRNLNGWYYDVKLRRKE |
| GLYUR000561S00023451.1 | 11-oxidase | | HPLPPGDMGWPLIGDLLSFIKDFSSGHPDSFINNLVLKYGRSGI |
| | | | YKTHLFGNPSIIVXEPQMCRRVLTDDVNFKLGYPKSIKELARC |
| | | | RPMIDVSNAEHRLFRRLITSPIVGHKALAMYLERLEEIVINSLE |
| | | | ELSSMKHPVELLKEMKKVSFKAIVHVFMGSSNQDIIKKIGSSF |
| | | | TDLYNGMFSIPINVPGFTFHKALEARKKLAKIVQPVVDERRLM |
| | | | IENGPQEGSQRKDLIDILLEVKDENGRKLEDEDISDLLIGLLFA |
| | | | GHESTATSLMWSITYLTQHPHILKKAKEEQEEITRTRFSSQKQL |
| | | | SLKEIKQMVYLSQVIDETLRCANIAFATFREATADVNINGYIIP |
| | | | KGWRVLIWARAIHMDSEYYPNPEEFNPSRWDDYNAKAGTFL |
| | | | PFGAGSRLCPGADLAKLEISIFLHYFLLNYRLERINPECHVTSLP |
| | | | VSXPTDNCLAKVIKVSCA |
| GuCYP72A154_ | 11-oxo- | 78 | ATGGATGCATCTTCCACACCAGGGGCTATCTGGGTTGTTCT |
| GLYUR000890S00019071.1 | β-amyrin | | GACAGTGATACTAGCTGCGATTCCCATATGGGCATGCCATA |
| | 30-oxidase | | TGGTGAACACGCTGTGGCTGAGGCCAAAGAGGTTGGAAAG |
| | | | GCATCTCAGAGCTCAAGGTCTTCATGGTGACCCTTACAAGC |
| | | | TCTCACTTGACAACTCCAAGCAAACCTATATGCTCAAGTTG |
| | | | CAACAAGAAGCACAATCAAAATCCATTGGTCTCTCCAAAG |
| | | | ATGATGCTGCACCACGAATCTTCTCCCTTGCCCATCAAACT |
| | | | GTACACAAATATGGAAAGAACTCCTTTGCATGGGAAGGGA |
| | | | CAGCACCAAAGGTGATCATCACAGACCCAGAGCAAATTAA |
| | | | GGAAGTCTTTAACAAGATTCAGGACTTCCCCAAACCAAAAT |
| | | | TAAATCCCATCGCCAAGTATATTAGCATCGGTCTAATACAG |
| | | | TATGAGGGTGACAAATGGGCCAAACATCGAAAGATTATCA |
| | | | ATCCGGCATTCCACTTAGAAAAATTGAAAGGTATGCTGCCA |
| | | | GCATTTTCTCATAGCTGCCATGAAATGATTAGCAAATGGAA |
| | | | GGGGTTATTGTCATCAGATGGAACATGTGAGGTTGATGTTT |
| | | | GGCCCTTCCTTCAAAATCTCACTTGTGATGTAATTTCTAGG |
| | | | ACGGCATTCGGAAGCAGCTATGCAGAAGGAGCAAAAATAT |
| | | | TTGAACTTTTGAAAAGGCAGGGATATGCTTTGATGACAGCA |
| | | | CGATACGCACGCATTCCATTATGGTGGCTTCTACCATCAAC |
| | | | TACCAAAAGGAGGATGAAGGAAATTGAAAGAGGCATACGT |
| | | | GATTCACTTGAAGGTATCATTAGAAAACGAGAAAAAGCAT |
| | | | TGAAGAGTGGCAAAAGCACCGATGACGACTTATTAGGCAT |
| | | | ACTTTTGCAATCAAATCACATTGAAAATAAAGGAGATGAA |
| | | | AACAGTAAGAGTGCTGGAATGACCACCCAAGAAGTAATGG |
| | | | AGGAATGCAAACTTTTTTACCTGGCAGGGCAAGAGNTTGA |
| | | | AAATAAAGGAGATGAAAACAGTAAGAGTGCTGGAATGACC |
| | | | ACCCAAGAAGTAATGGAGGAATGCAAACTTTTTTACCTGGC |
| | | | AGGGCAAGAGACCACCGCAGCTTTGCTGGCCTGGACAATG |
| | | | GTGTTATTAGGCAAGCATCCTGAATGGCAAGCACGCGCAA |
| | | | GGCAGGAAGTTTTGCAAGTAACCATGATTTTATATGAGGTA |
| | | | CTCAGGCTGTACCCACCTGGGATTTACCTCACCCGAGCTCT |
| | | | TCGAAAGGATTTGAAACTTGGAAACCTTTTGCTACCTGCTG |
| | | | GAGTACAGGTTTCCGTACCAATACTTTTGATTCACCATGAT |
| | | | GAAGGTATATGGGGCAATGATGCAAAGGAGTTCAATCCTG |
| | | | AAAGGTTTGCTGAAGGAATTGCAAAGGCAACAAAAGGCCA |
| | | | AGTTTGCTATTTCCCTTTTGGATGGGGTCCTAGAATATGTGT |
| | | | TGGGCAAAACTTTGCCTTATTAGAAGCCAAGATTGTATTGT |
| | | | CATTGCTGCTGCAGAATTTCTCATTTGAGCTATCTCCGACTT |
| | | | ATGCACATGTTCCTACCACGGTGCTTACTTTGCAGCCAAAA |
| | | | CATGGGGCACCCATCATTCTGCATAAACTGTAA |
| GuCYP72A154_ | 11-oxo- | 79 | MDASSTPGAIWVVLTVILAAIPIWACHMVNTLWLRPKRLERH |
| GLYUR000890S00019071.1 | β-amyrin | | LRAQGLHGDPYKLSLDNSKQTYMLKLQQEAQSKSIGLSKDDA |
| | 30- | | APRIFSLAHQTVHKYGKNSFAWEGTAPKVIITDPEQIKEVENKI |
| | oxidase | | QDFPKPKLNPIAKYISIGLIQYEGDKWAKHRKIINPAFHLEKLK |
| | | | GMLPAFSHSCHEMISKWKGLLSSDGTCEVDVWPFLQNLTCDV |
| | | | ISRTAFGSSYAEGAKIFELLKRQGYALMTARYARIPLWWLLPS |
| | | | TTKRRMKEIERGIRDSLEGIIRKREKALKSGKSTDDDLLGILLQ |
| | | | SNHIENKGDENSKSAGMTTQEVMEECKLFYLAGQEXENKGD |
| | | | ENSKSAGMTTQEVMEECKLFYLAGQETTAALLAWTMVLLGK |
| | | | HPEWQARARQEVLQVTMILYEVLRLYPPGIYLTRALRKDLKL |
| | | | GNLLLPAGVQVSVPILLIHHDEGIWGNDAKEFNPERFAEGIAK |
| | | | ATKGQVCYFPFGWGPRICVGQNFALLEAKIVLSLLLQNFSFEL |
| | | | SPTYAHVPTTVLTLQPKHGAPIILHKL |
| GuCSL_ | Cellulose | 80 | ATGGCAAGCTTCACCCTTCACACAGAAACCGTTCAGTCATG |
| GLYUR003152S00037491.1 | Synthase | | GCTACTCCTCAGCAGACTTCACATACTGCTGCACCTCGCAG |
| (SOAP5 homolog; | Like G | | TTGTACTGCTCCTCTTATACTACCGCATCACACGTTTCCCCT |
| Cellulose Synthase | (Glucuronic | | TCCATGCTCCSACTCTACCGTGGACTCTGATGACCGTAGGT |
| Like G) | acid | | GAGGCTATTATGGCAGTGCTGTGGTTCTTCAACCAGGCCTT |
| | transferase) | | CCGGTGGCGGCCGGTGAGCCGCTCGGTGATGACGGAGAAG |
| | | | CTGCCCAGCGACGCGAAGCTGCCGGGGCTTGACATATTCGT |
| | | | GTGCACGCTTGACCCCGAGAAGGAGCCCACCGTGGAGGTG |
| | | | ATGAACACTCTGGTCTCTGCCCTTGCCATGGACTACCCCCC |
| | | | TGACAAGCTCTCCGTTTACCTCTCCGACGATGGCGCCGCCC |
| | | | CGGTCACTCTTTACGGCGTGAGAGAGGCTTCTGAGTTCGCG |
| | | | AGGGTGTGGGTCCCTTTCTGCAAAAAGTATGGGATCAAGTC |
| | | | AAGGTGTCCCAAGGTTTTCTTCTCTCCCAGTGCTGAGGATG |
| | | | AACACCTTCTTCGCACCGACGAGTTCAGGTCAGAGCGAGA |
| | | | CCTCATCAAGGCTAAATACGAGAAAATGCAGAAAAATATT |
| | | | GAGAAATTTGGTTCGGATGCCAAAAATTGTCGTATGGTGAC |
| | | | TGACAGACCTCCTCGGATCGAGATATTGATTGACCAACCAG |
| | | | ACATGCCACGTGTTGTTTACGTGTCTCGGGAAAGAAGGCCA |
| | | | TCACTCCCTCACAAGTTCAAAGGAGGAGCCCTCAATACATT |
| | | | GCTCAGAGTCTCAGGTCTAATCAGCAATGGGCCTTATGTAC |
| | | | TTGTAGTGGACTGTGATATGTATTGCAATGACCCATCCTCA |
| | | | GCCAAACAAGCCATGTGTTTCTTTCTTGATCCTGAAACCTC |
| | | | TAAATYTATTGCATTTGTCCAATTCCCTCAAATGTTTCACAA |
| | | | CCTTGGCAAAAAAGACATCTATGACAATCAATCTAGGACT |
| | | | GCTTTTAAGACAATGTGGCAAGGGATGGATGGACTAAGAG |
| | | | GTCCTGGTCTTTCTGGCAGCGGTAATTACTTGAATAGAAGT |
| | | | GCATTACTATTTGGAAGTCCAAATCAAAAAGATGACTATCT |
| | | | GGATGATGCCCAAAACTACTTRGGCAAGTCTACCATGTACA |
| | | | TAGAATCACTAAAGGCCATTCGTGGACAAAAAACTATGAA |
| | | | AAAGAATATTTCAAGAGATGAAATTTTACGAGAAGCTCAA |
| | | | GTATTAGCCTCTTGTTCCTATGAGACAAACACAGAATGGGG |
| | | | AGCAGAGGTAGGATTCTCATATGGCATCTTACTGGAGAGTT |
| | | | CAATCACTGGCTATCTTTTYCACTGCAGAGGATGGAAATCA |
| | | | GCATATCTTTACCCAAAGACACCATGTTTCTTAGGGTGTGC |
| | | | CCCAACTGACATCAAGGAAGGAATGCTCCAATTGGTGAAG |
| | | | TGGTTGTCTGAATACTGCTTGCTRGGATTCTCTAAATACAG |
| | | | CCCTTTCACTTATGGCTTTTCAAGAATGCCCATTATGCCTAC |
| | | | CTTAGTCTATTGCTTCTTGACAACWACAACCCTTTATTCCA |
| | | | TTGTCTTCATCCTTTATGGCATTGTCCCCCAAGTTTGCTTCT |
| | | | TAAAAGGAATACCCGTGTTTCCAAAGGTCACAGACCCTTGG |
| | | | TTTGCAGTGTTTGCAACACTGTATATATCCACCCAGATTCA |
| | | | ACATTTGATAGAGGTCCTTTCTGGTGATGGCTCTGTGGCAA |
| | | | TGTGGTGGGATGAACAGKGAATCTGGATTCTGAAGTCAGT |
| | | | CACTAGCGTGTTCGCAATCATAGAGGCAGCTAAGAAAGGG |
| | | | TTAGGATTGAACAAGAAGAAATTCATGTTGTCAAACAAAG |
| | | | CAATTGACAAGGAGAAGCTCAAGAAGTATGAGCAAGGTAG |
| | | | GTTTGATTTCCAAGGTGCAGCTCTGTTCATGTCCCCAATGG |
| | | | TTGTGTTGCTCATAGTGAACGTTGTTTCCTTCATTGGTGGCA |
| | | | TATGGAGACTATTCAATGCAAAGGATATTGAAGATATGTTT |
| | | | GGTCAGCTTTTCCTAGTTAGTTATGTAATGGCCCTTAGTTAT |
| | | | CCCATTTTTGAAGGGATAATAACCATGAAAAGCAAGAGTG |
| | | | GATAG |
| GuCSLG_ | Cellulose | 81 | MASFTLHTETVQSWLLLSRLHILLHLAVVLLLLYYRITR |
| GLYUR003152S00037491.1 | Synthase | | FPFHAPTLPWTLMTVGEAIMAVLWFFNQAFRWRPVSRS |
| (SOAP5 homolog; | Like G | | VMTEKLPSDAKLPGLDIFVCTLDPEKEPTVEVMNTLVSA |
| Cellulose Synthase | (Glucuronic | | LAMDYPPDKLSVYLSDDGAAPVTLYGVREASEFARVW |
| Like G) | acid | | VPFCKKYGIKSRCPKVFFSPSAEDEHLLRTDEFRSERDLI |
| | transferase) | | KAKYEKMQKNIEKFGSDAKNCRMVTDRPPRIEILIDQPD |
| | | | MPRVVYVSRERRPSLPHKFKGGALNTLLRVSGLISNGPY |
| | | | VLVVDCDMYCNDPSSAKQAMCFFLDPETSKXIAFVQFP |
| | | | QMFHNLGKKDIYDNQSRTAFKTMWQGMDGLRGPGLS |
| | | | GSGNYLNRSALLFGSPNQKDDYLDDAQNYXGKSTMYIE |
| | | | SLKAIRGQKTMKKNISRDEILREAQVLASCSYETNTEWG |
| | | | AEVGFSYGILLESSITGYLXHCRGWKSAYLYPKTPCFLG |
| | | | CAPTDIKEGMLQLVKWLSEYCLLGFSKYSPFTYGFSRMP |
| | | | IMPTLVYCFLTTTTLYSIVFILYGIVPQVCFLKGIPVFPKV |
| | | | TDPWFAVFATLYISTQIQHLIEVLSGDGSVAMWWDEQX |
| | | | IWILKSVTSVFAIIEAAKKGLGLNKKKFMLSNKAIDKEK |
| | | | LKKYEQGRFDFQGAALFMSPMVVLLIVNVVSFIGGIWR |
| | | | LFNAKDIEDMFGQLFLVSYVMALSYPIFEGIITMKSKSG |
| GuUGAT_ | Glycyrrhetinic | 82 | ATGACCATGGGTAACGAGAATCGGGAGCTGCACATAATCT |
| KT759000.1 | acid | | TCTTCCCCTTTCTGGCGAACGGCCACATCATCCCCTGCGTG |
| | glucuronosyl- | | GACTTGGCCAGAGTCTTCGCCGCAAGAGGAATCAGAGCCA |
| | transferase | | CCATAGTCACCACCCACCTCAACGTTCCCTACATTTCCAGA |
| | | | ACCATCGGAAAAGCCAACATCAACATCAGAACCATCAAGT |
| | | | TCCCTTCCACCGAAGACTCTGGCCTTCCCGAAGGCTGCGAG |
| | | | AATACCGAGTCAGCACTCGCCCCTGACAAGTTCATCAAGTT |
| | | | CATGAAGGCCACCCTGCTCCTGAGGGACCCACTTGAACAC |
| | | | GTGTTACAGGAAGAGCAACCACACTCTTGGTCGCCGACAT |
| | | | GTTCTTCCCTTGGGCCACCGACTCCGCCGCAAAATTCGGCA |
| | | | TCCCTAGGATCGTGTTCCACGGCCTCGGTTACTTCCCACTCT |
| | | | GCGTTCTTGCATGCACGAGACAGTACAAGCCTCAGGACAA |
| | | | GGTTTCATCTTACACGGAACCCTTCGTGGTTCCGAATCTCC |
| | | | CGGGTGAAATAACACTGACGAAGATGCAGCTGCCGCAGTT |
| | | | GCCTCAGCACGACAAGGTCTTCACCCAGTTGTTGGAAGAGT |
| | | | CAAACGAATCGGAGTTGAAGAGCTTCGGTGTGATTGTAAA |
| | | | CAGCTTCTACGAACTTGAACCGGTTTACGCGGATCATTACA |
| | | | GGAACGAGCTTGGGAGAAGAGCTTGGCATTTGGGTCCGGT |
| | | | TTCATTATGCAGTAGGGACACGGAGGAAAAATCGCGGAGG |
| | | | GGAAGGGAAGCTGCAATTGATGAGAACGAGTGCTTGAAGT |
| | | | GGCTTCAATCAAAGGAACCCAATTCGGTTGTTTATGTTTGT |
| | | | TTCGGTAGCATGATGGTTTTCAGTGACGCTCAGCTAAAAGA |
| | | | GATTGCGATGGGTCTTGAGGCTTCAGGGAAGCCATTCATAT |
| | | | GGGTGGTGAAGAAAGGAGGGGCTAAAAGTGAAGGTGAGA |
| | | | AATTGGAGTGGCTTCCAGAAGGGTTTGAGGAGAGAATGGG |
| | | | GGAAAGTAATAAGGGACTAATCATAAGGGGTTGGGCACCA |
| | | | CAGGTGATGATTTTGGACCATGGAGCGGTTGGAGGGTTTGT |
| | | | GACACATTGTGGGTGGAATTCAACGCTGGAAGGAGTGTGT |
| | | | GCAGGGGTGCCAATGGTGACTTGGCCCATGTATGGGGAAC |
| | | | AATTTTACAACGCCAAGTTTCTGACGGACATAGTGAAAATT |
| | | | GGGGTGGGTGTTGGGGTTCAAACGTGGATTGGGATGGGAG |
| | | | GAGGAGAGCCTGTGAAGAAGGAAGTGATAGAGCAGGCAG |
| | | | TGAGAAGGATAATGGTGGGGCAGGAAGCAGAGGAAATGA |
| | | | GAAACAGAGCCAAGGAACTGAGCCAGATGGCAAAGCGTGC |
| | | | TGTGGAGGAAGGAGGATCGTCTCACAACGATTTTAACTCTT |
| | | | TAATTGAGGATTTGAGGTCGCGTGCCCATTAA |
| GuUGAT_ | Glycyrrhetinic | 83 | MTMGNENRELHIIFFPFLANGHIIPCVDLARVFAARGIRATIVT |
| KT759000.1 | acid | | THLNVPYISRTIGKANINIRTIKFPSTEDSGLPEGCENTESALAP |
| | glucuronosyl- | | DKFIKFMKATLLLRDPLEHVLQEEQPHCLVADMFFPWATDSA |
| | transferase | | AKFGIPRIVFHGLGYFPLCVLACTRQYKPQDKVSSYTEPFVVP |
| | | | NLPGEITLTKMQLPQLPQHDKVFTQLLEESNESELKSFGVIVNS |
| | | | FYELEPVYADHYRNELGRRAWHLGPVSLCSRDTEEKSRRGRE |
| | | | AAIDENECLKWLQSKEPNSVVYVCFGSMMVFSDAQLKEIAM |
| | | | GLEASGKPFIWVVKKGGAKSEGEKLEWLPEGFEERMGESNK |
| | | | GLIIRGWAPQVMILDHGAVGGFVTHCGWNSTLEGVCAGVPM |
| | | | VTWPMYGEQFYNAKFLTDIVKIGVGVGVQTWIGMGGGEPVK |
| | | | KEVIEQAVRRIMVGQEAEEMRNRAKELSQMAKRAVEEGGSS |
| | | | HNDFNSLIEDLRSRAH |
| GuUGT73P12_ | Glycyrrhetinic | 84 | ATGGACTCCTTTGGGGTTGAAGGTGATCACCAAGCCGACAC |
| SCAFFOLD00629 | acid 3-O- | | CACAGTGCTGAAGGCGGTTTTTCTTCCCTTCATCTCAAAAA |
| (LC314779) | monoglucuronide | | GTCATCTCATCCGTGAGGTGGACAAAGCAAGGATCTTCGCC |
| | glucuronosyl- | | ATGCACGGCGTGGATGTCACCATCATCACCACGCCGGCCA |
| | transferase | | ACGCTGCCACTTTCCAAACCTCCATTGACCGCGACTCCAGC |
| | | | CGCGGCCGCTCCATCAGAACGCACATCGTTCCGTTCCCCCA |
| | | | AGTCCCCGGTCTACCACAGGGACTCGAGAGACTCGACGCC |
| | | | GACACTCCTCAACACTTGCTCTCCAAGATCTACCATGGACT |
| | | | ATCCATTCTGCAAGAGCAGTTCCAACAACTGTTCCGTGAAA |
| | | | TGAGGCCAGATTTCATAGTCACTGACATGTACTACCCTTGG |
| | | | AGCGTCGATGCCGCCGCCGAGTTGGGGATTCCGAGGTTGGT |
| | | | TTGTAACGGTGGAAGCTACTTCGCTCAGTCAGCTGTTAACT |
| | | | CCGTTGAGCTATTTTCACCACAAGCCAAGGTTGATTCAAAT |
| | | | ACCGAGACTTTTCTGCTTCCTGGGTTACCCCATGAGGTTGA |
| | | | GATGACACGTTTGCAACTACCGGATTGGCTTAGAGGAGCA |
| | | | CCGAATGAGTACACCTATTTGATGAAGATGATCAAGGATTC |
| | | | AGAGAGGAAGAGTTATGGGTCATTGTTCAATAGCTTTTATG |
| | | | AGCTTGAAGGGACTTATGAGGAACATTACAAGAAAGCCAT |
| | | | GGGAACCAAGAGTTGGAGTGTGGGGCCAGTTTCTTTGTGG |
| | | | GTGAACCAAGATGCTTCTGATAAGGCTTGTAGGGGGGATG |
| | | | TTAAAGAAGGAAAAGGAGATGGGGTGGTGCTTACTTGGCT |
| | | | GGATTCTAAAACAGAGGACTCTGTTTTGTATGTGAGTTTTG |
| | | | GGAGCATGAACAAGTTCCCTAAAACTCAGCTTGTTGAGATA |
| | | | GCTCATGCCCTCGAAGATTCTGGCCATGATTTCATTTGGGT |
| | | | CGTTGGCAAAATTGAAGAAGGTGAAGGTGGTGCTGATTTTT |
| | | | TGAGGGAATTTGAGAAGAAAGTGAAAGAAAAAAACAGAG |
| | | | GTTATCTGATATGGGGTTGGGCACCACAGCTTCTGATTCTG |
| | | | GAGCATCCTGCGGTTGGAGCAGTGGTGACTCATTGTGGGTG |
| | | | GAACACCGTTATGGAAAGTGTGAATGCAAGTTTGCCATTGG |
| | | | CAACTTGGCCATTGTTTGCGGAGCAGTTCTTCAATGAGAAG |
| | | | CTAGTGGTTGATGTGGTGAAGATTGGTGTGCCAGTTGGGGT |
| | | | TAAGGAATGGAGAAATTGGAATGAGTTTGGGGATGAGGTT |
| | | | GTGAAGAGGGAGGACATAGGAAAGGCCATTGCTTTTTTGA |
| | | | TGGGTGGTGGGGATGAATCCTTGGAAATGAGGAAGAGGGT |
| | | | CAAGGTGCTCAGTGGTGCTACAAAGAAAGCTATTCAGGTT |
| | | | GGTGGGTCTTCTCACACCAAGTTGAAAGAACTCATAGAAG |
| | | | AGCTCAAGTCAATCAAGCTACAAAAGGTCAACAACAAATT |
| | | | AATGGAGGCAGTGGCTTAA |
| GuUGT73P12_ | Glycyrrhetinic | 85 | MDSFGVEGDHQADTTVLKAVFLPFISKSHLIREVDKARI |
| SCAFFOLD00629 | acid 3-O- | | FAMHGVDVTIITTPANAATFQTSIDRDSSRGRSIRTHIVPF |
| (LC314779) | monoglucuronide | | PQVPGLPQGLERLDADTPQHLLSKIYHGLSILQEQFQQLF |
| | glucuronosyl- | | REMRPDFIVTDMYYPWSVDAAAELGIPRLVCNGGSYFA |
| | transferase | | QSAVNSVELFSPQAKVDSNTETFLLPGLPHEVEMTRLQL |
| | | | PDWLRGAPNEYTYLMKMIKDSERKSYGSLFNSFYELEG |
| | | | TYEEHYKKAMGTKSWSVGPVSLWVNQDASDKACRGD |
| | | | VKEGKGDGVVLTWLDSKTEDSVLYVSFGSMNKFPKTQ |
| | | | LVEIAHALEDSGHDFIWVVGKIEEGEGGADFLREFEKKV |
| | | | KEKNRGYLIWGWAPQLLILEHPAVGAVVTHCGWNTVM |
| | | | ESVNASLPLATWPLFAEQFFNEKLVVDVVKIGVPVGVK |
| | | | EWRNWNEFGDEVVKREDIGKAIAFLMGGGDESLEMRK |
| | | | RVKVLSGATKKAIQVGGSSHTKLKELIEELKSIKLQKVN |
| | | | NKLMEAVA |
| GuCSLG | Cellulose | 102 | MASFTLHTETVQSWLLLSRLHILLHLAVVLLLLYYRITR |
| Protein from | Synthase | | FPFHAPTLPWTLMTVGEAIMAVLWFFNQAFRWRPVSRS |
| Chinese | Like G | | VMTEKLPSDAKLPGLDIFVCTLDPEKEPTVEVMNTLVSA |
| liquorice_ | (Glucuronic | | LAMDYPPDKLSVYLSDDGAAPVTLYGVREASEFARVW |
| (Glyur003152s00037491.1 | acid | | VPFCKKYGIKSRCPKVFFSPSAEDEHLLRTDEFRSERDLI |
| Glycyrrhiza | transferase) | | KAKYEKMQKNIEKFGSDAKNCRMVTDRPPRIEILIDQPD |
| uralensis) | | | MPRVVYVSRERRPSLPHKFKGGALNTLLRVSGLISNGPY |
| (SOAP5 homolog; | | | VLVVDCDMYCNDPSSAKQAMCFFLDPETSKSIAFVQFP |
| Cellulose Synthase | | | QMFHNLGKKDIYDNQSRTAFKTMWQGMDGLRGPGLS |
| Like G) | | | GSGNYLNRSALLFGSPNQKDDYLDDAQNYLGKSTMYIE |
| | | | SLKAIRGQKTMKKNISRDEILREAQVLASCSYETNTEWG |
| | | | AEVGFSYGILLESSITGYLFHCRGWKSAYLYPKTPCFLG |
| | | | CAPTDIKEGMLQLVKWLSEYCLLGFSKYSPFTYGFSRMP |
| | | | IMPTLVYCFLTTTTLYSIVFILYGIVPQVCFLKGIPVFPKV |
| | | | TDPWFAVFATLYISTQIQHLIEVLSGDGSVAMWWDEQG |
| | | | IWILKSVTSVFAIIEAAKKGLGLNKKKFMLSNKAIDKEK |
| | | | LKKYEQGRFDFQGAALFMSPMVVLLIVNVVSFIGGIWR |
| | | | LFNAKDIEDMFGQLFLVSYVMALSYPIFEGIITMKSKSG |
| GuCSLG | Cellulose | 103 | ATGGCAAGCTTCACCCTTCACACAGAAACCGTTCAGT |
| Gene from | Synthase | | CATGGCTACTCCTCAGCAGACTTCACATACTGCTGCA |
| Chinese | Like G | | CCTCGCAGTTGTACTGCTCCTCTTATACTACCGCATCA |
| liquorice_ | (Glucuronic | | CACGTTTCCCCTTCCATGCTCCGACTCTACCGTGGACT |
| (Glyur003152s00037491.1_ | acid | | CTGATGACCGTAGGTGAGGCTATTATGGCAGTGCTGT |
| Glycyrrhiza | transferase) | | GGTTCTTCAACCAGGCCTTCCGGTGGCGGCCGGTGAG |
| uralensis) | | | CCGCTCGGTGATGACGGAGAAGCTGCCCAGCGACGC |
| (SOAP5 homolog; | | | GAAGCTGCCGGGGCTTGACATATTCGTGTGCACGCTT |
| Cellulose Synthase | | | GACCCCGAGAAGGAGCCCACCGTGGAGGTGATGAAC |
| Like G) | | | ACTCTGGTCTCTGCCCTTGCCATGGACTACCCCCCTGA |
| | | | CAAGCTCTCCGTTTACCTCTCCGACGATGGCGCCGCC |
| | | | CCGGTCACTCTTTACGGCGTGAGAGAGGCTTCTGAGT |
| | | | TCGCGAGGGTGTGGGTCCCTTTCTGCAAAAAGTATGG |
| | | | GATCAAGTCAAGGTGTCCCAAGGTTTTCTTCTCTCCCA |
| | | | GTGCTGAGGATGAACACCTTCTTCGCACCGACGAGTT |
| | | | CAGGTCAGAGCGAGACCTCATCAAGGCTAAATACGA |
| | | | GAAAATGCAGAAAAATATTGAGAAATTTGGTTCGGAT |
| | | | GCCAAAAATTGTCGTATGGTGACTGACAGACCTCCTC |
| | | | GGATCGAGATATTGATTGACCAACCAGACATGCCACG |
| | | | TGTTGTTTACGTGTCTCGGGAAAGAAGGCCATCACTC |
| | | | CCTCACAAGTTCAAAGGAGGAGCCCTCAATACATTGC |
| | | | TCAGAGTCTCAGGTCTAATCAGCAATGGGCCTTATGT |
| | | | ACTTGTAGTGGACTGTGATATGTATTGCAATGACCCA |
| | | | TCCTCAGCCAAACAAGCCATGTGTTTCTTTCTTGATCC |
| | | | TGAAACCTCTAAATCTATTGCATTTGTCCAATTCCCTC |
| | | | AAATGTTTCACAACCTTGGCAAAAAAGACATCTATGA |
| | | | CAATCAATCTAGGACTGCTTTTAAGACAATGTGGCAA |
| | | | GGGATGGATGGACTAAGAGGTCCTGGTCTTTCTGGCA |
| | | | GCGGTAATTACTTGAATAGAAGTGCATTACTATTTGG |
| | | | AAGTCCAAATCAAAAAGATGACTATCTGGATGATGCC |
| | | | CAAAACTACTTAGGCAAGTCTACCATGTACATAGAAT |
| | | | CACTAAAGGCCATTCGTGGACAAAAAACTATGAAAA |
| | | | AGAATATTTCAAGAGATGAAATTTTACGAGAAGCTCA |
| | | | AGTATTAGCCTCTTGTTCCTATGAGACAAACACAGAA |
| | | | TGGGGAGCAGAGGTAGGATTCTCATATGGCATCTTAC |
| | | | TGGAGAGTTCAATCACTGGCTATCTTTTCCACTGCAG |
| | | | AGGATGGAAATCAGCATATCTTTACCCAAAGACACCA |
| | | | TGTTTCTTAGGGTGTGCCCCAACTGACATCAAGGAAG |
| | | | GAATGCTCCAATTGGTGAAGTGGTTGTCTGAATACTG |
| | | | CTTGCTAGGATTCTCTAAATACAGCCCTTTCACTTATG |
| | | | GCTTTTCAAGAATGCCCATTATGCCTACCTTAGTCTAT |
| | | | TGCTTCTTGACAACAACAACCCTTTATTCCATTGTCTT |
| | | | CATCCTTTATGGCATTGTCCCCCAAGTTTGCTTCTTAA |
| | | | AAGGAATACCCGTGTTTCCAAAGGTCACAGACCCTTG |
| | | | GTTTGCAGTGTTTGCAACACTGTATATATCCACCCAG |
| | | | ATTCAACATTTGATAGAGGTCCTTTCTGGTGATGGCTC |
| | | | TGTGGCAATGTGGTGGGATGAACAGGGAATCTGGATT |
| | | | CTGAAGTCAGTCACTAGCGTGTTCGCAATCATAGAGG |
| | | | CAGCTAAGAAAGGGTTAGGATTGAACAAGAAGAAAT |
| | | | TCATGTTGTCAAACAAAGCAATTGACAAGGAGAAGCT |
| | | | CAAGAAGTATGAGCAAGGTAGGTTTGATTTCCAAGGT |
| | | | GCAGCTCTGTTCATGTCCCCAATGGTTGTGTTGCTCAT |
| | | | AGTGAACGTTGTTTCCTTCATTGGTGGCATATGGAGA |
| | | | CTATTCAATGCAAAGGATATTGAAGATATGTTTGGTC |
| | | | AGCTTTTCCTAGTTAGTTATGTAATGGCCCTTAGTTAT |
| | | | CCCATTTTTGAAGGGATAATAACCATGAAAAGCAAGA |
| | | | GTGGATAG |

Expression of spinach bAS (SoSOAP1; SEQ ID NO: 45), GuCYP88D6 (SEQ ID NO: 76), GuCYP72A154 (SEQ TD NO: 78), and GuCSL (SEQ TD NO: 80) in N. benthamiana resulted in formation of glycyrrhetinic acid 3-O-monoglucuronide (GA-3-GlcA) (FIGS. 46A-46E). On the other hand, previously reported GuUGAT (SEQ TD NO: 82) believed to catalyze the continuous two-step glucuronidation of glycyrrhetinic acid to yield glycyrrhizin failed to attach GlcA to the aglycone in the assays performed here (G. Xu, W. Cai, W. Gao, C Liu, A novel glucuronosyltransferase has an unprecedented ability to catalyse continuous two-step glucuronosylation of glycyrrhetinic acid to yield glycyrrhizin. New Phytol. 212, 123-35 (2016).).

Transient expression of GuUGT73P12 (SEQ ID NO: 84), which was previously shown to attach the second GlcA, together with GuCSL and other enzymes gave glycyrrhizin as a final product in N. benthamiana (FIGS. 47A-47F). The assays also showed that GuCSL (SEQ ID NO: 81) can perform addition of the second glucuronic acid moiety to GA-3-GlcA, but with very low efficiency.

Furthermore, it will be shown in a heterologous system (N. benthamiana) that the first glycosylation step in the biosynthesis of QS-21 from Q. saponaria may be performed by a CSLG which attaches glucuronic acid to the quillaic acid at position C-3.

Summary: The discovery of a new type of biosynthetic reaction performed by cellulose synthase like G enzymes provides a valuable strategy for engineering pathways of important specialized metabolites and their sustainable production in heterologous systems (plant or fungal).

### Example 24: Structural Analysis of Cellulose Synthase Like G

Objective: To analyze the structure of Cellulose Synthase Like G (CSLG) and compare it with the classical Cellulose Synthase A (CESA).

Methods: See Materials and Methods above.

Results: Phylogenetic analysis showed that CSLGs are evolutionarily distinct from the classical Cellulose Synthase A (CESA) enzymes that mediate cellulose biosynthesis (approximately 30% amino acid sequence similarity). However, these two protein groups share several structural features. A predicted 3D model obtained by template-based tertiary structure modelling of the spinach SOAP5 CSLG protein showed that it consists of two domains traversing a lipid bilayer (TMD) at the amino-termini and an additional four TMDs at the carboxy terminal end (M. Källberg et al., Template-based protein structure modeling using the RaptorX web server. Nature Protocols. 7, 1511-1522 (2012).). A 445 amino acid long loop protruding from the lipid bilayer is located between the transmembrane domains (FIG. 48; FIG. 49A). The amino acid sequences of the spinach SOAP5 enzyme, the cellulose synthase like G enzyme from Arabidopsis, and the cellulose synthase A enzyme subunits from Arabidopsis and their orthologs in spinach are presented below in Table 19. In addition, other cellulose synthase like genes are presented from the A, B, and E families. The current understanding is that cellulose synthase like genes from A, B, and E families each have completely different activities. Until the work presented here, only few CSLs were known, and none from CSLG family, were characterized. Those that were analyzed are involved in sugar metabolism, biosynthesis of beta-glucans and hemicelluloses. It was never shown that any CSL (despite family it belongs) can work on triterpenoids. Thus, the results presented throughout the examples provided surprising and unexpected activities for a cellulose like synthase.

TABLE 19
Amino Acid and Nucleotide Sequences of
Arabidopsis CSLG and CESA enzymes

| | | SEQ | |
|---|---|---|---|
| | ENZYME | ID | |
| NAME | ACTIVITY | NO: | AA SEQ |
| Arabidopsis | Cellulose | 67 | MEASAGLVAGSYRRNELVRIRHESDGGTKPLKNMN |
| CESA1 | Synthase A | | GQICQICGDDVGLAETGDVFVACNECAFPVCRPCYE |
| | (subunit 1) | | YERKDGTQCCPQCKTRFRRHRGSPRVEGDEDEDDV |
| | | | DDIENEFNYAQGANKARHQRHGEEFSSSSRHESQPIP |
| | | | LLTHGHTVSGEIRTPDTQSVRTTSGPLGPSDRNAISSP |
| | | | YIDPRQPVPVRIVDPSKDLNSYGLGNVDWKERVEGW |
| | | | KLKQEKNMLQMTGKYHEGKGGEIEGTGSNGEELQM |
| | | | ADDTRLPMSRVVPIPSSRLTPYRVVIILRLIILCFFLQY |
| | | | RTTHPVKNAYPLWLTSVICEIWFAFSWLLDQFPKWY |
| | | | PINRETYLDRLAIRYDRDGEPSQLVPVDVFVSTVDPL |
| | | | KEPPLVTANTVLSILSVDYPVDKVACYVSDDGSAML |
| | | | TFESLSETAEFAKKWVPFCKKFNIEPRAPEFYFAQKID |
| | | | YLKDKIQPSFVKERRAMKREYEEFKVRINALVAKAQ |
| | | | KIPEEGWTMQDGTPWPGNNTRDHPGMIQVFLGHSG |
| | | | GLDTDGNELPRLIYVSREKRPGFQHHKKAGAMNALI |
| | | | RVSAVLTNGAYLLNVDCDHYFNNSKAIKEAMCFMM |
| | | | DPAIGKKCCYVQFPQRFDGIDLHDRYANRNIVFFDIN |
| | | | MKGLDGIQGPVYVGTGCCFNRQALYGYDPVLTEED |
| | | | LEPNIIVKSCCGSRKKGKSSKKYNYEKRRGINRSDSN |
| | | | APLFNMEDIDEGFEGYDDERSILMSQRSVEKRFGQSP |
| | | | VFIAATFMEQGGIPPTTNPATLLKEAIHVISCGYEDKT |
| | | | EWGKEIGWIYGSVTEDILTGFKMHARGWISIYCNPPR |
| | | | PAFKGSAPINLSDRLNQVLRWALGSIEILLSRHCPIWY |
| | | | GYHGRLRLLERIAYINTIVYPITSIPLIAYCILPAFCLIT |
| | | | DRFIIPEISNYASIWFILLFISIAVTGILELRWSGVSIED |
| | | | WWRNEQFWVIGGTSAHLFAVFQGLLKVLAGIDTNFT |
| | | | VTSKATDEDGDFAELYIFKWTALLIPPTTVLLVNLIGI |
| | | | VAGVSYAVNSGYQSWGPLFGKLFFALWVIAHLYPFL |
| | | | KGLLGRQNRTPTIVIVWSVLLASIFSLLWVRINPFVDA |
| | | | NPNANNFNGKGGVF |
| Arabidopsis | Cellulose | 68 | MESEGETAGKPMKNIVPQTCQICSDNVGKTVDGDRF |
| CESA3 | Synthase A | | VACDICSFPVCRPCYEYERKDGNQSCPQCKTRYKRL |
| | (subunit 3) | | KGSPAIPGDKDEDGLADEGTVEFNYPQKEKISERML |
| | | | GWHLTRGKGEEMGEPQYDKEVSHNHLPRLTSRQDT |
| | | | SGEFSAASPERLSVSSTIAGGKRLPYSSDVNQSPNRRI |
| | | | VDPVGLGNVAWKERVDGWKMKQEKNTGPVSTQAA |
| | | | SERGGVDIDASTDILADEALLNDEARQPLSRKVSIPSS |
| | | | RINPYRMVIMLRLVILCLFLHYRITNPVPNAFALWLV |
| | | | SVICEIWFALSWILDQFPKWFPVNRETYLDRLALRYD |
| | | | REGEPSQLAAVDIFVSTVDPLKEPPLVTANTVLSILAV |
| | | | DYPVDKVSCYVFDDGAAMLSFESLAETSEFARKWVP |
| | | | FCKKYSIEPRAPEWYFAAKIDYLKDKVQTSFVKDRR |
| | | | AMKREYEEFKIRINALVSKALKCPEEGWVMQDGTP |
| | | | WPGNNTGDHPGMIQVFLGQNGGLDAEGNELPRLVY |
| | | | VSREKRPGFQHHKKAGAMNALVRVSAVLTNGPFILN |
| | | | LDCDHYINNSKALREAMCFLMDPNLGKQVCYVQFP |
| | | | QRFDGIDKNDRYANRNTVFFDINLRGLDGIQGPVYV |
| | | | GTGCVFNRTALYGYEPPIKVKHKKPSLLSKLCGGSR |
| | | | KKNSKAKKESDKKKSGRHTDSTVPVFNLDDIEEGVE |
| | | | GAGFDDEKALLMSQMSLEKRFGQSAVFVASTLMEN |
| | | | GGVPPSATPENLLKEAIHVISCGYEDKSDWGMEIGWI |
| | | | YGSVTEDILTGFKMHARGWRSIYCMPKLPAFKGSAPI |
| | | | NLSDRLNQVLRWALGSVEILFSRHCPIWYGYNGRLK |
| | | | FLERFAYVNTTIYPITSIPLLMYCTLLAVCLFTNQFIIP |
| | | | QISNIASIWFLSLFLSIFATGILEMRWSGVGIDEWWRN |
| | | | EQFWVIGGVSAHLFAVFQGILKVLAGIDTNFTVTSKA |
| | | | SDEDGDFAELYLFKWTTLLIPPTTLLIVNLVGVVAGV |
| | | | SYAINSGYQSWGPLFGKLFFAFWVIVHLYPFLKGLM |
| | | | GRQNRTPTIVVVWSVLLASIFSLLWVRIDPFTSRVTGP |
| | | | DILECGINC |
| Arabidopsis | Cellulose | 69 | METHRKNSVVGNILHTCHPCRRTIPYRIYAIFHTCGII |
| CSLG1 | Synthase | | ALMYHHVHSLVTANNTLITCLLLLSDIVLAFMWATT |
| | Like G | | TSLRLNPVHRTECPEKYAAKPEDFPKLDVFICTADPY |
| | | | KEPPMMVVNTALSVMAYEYPSDKISVYVSDDGGSSL |
| | | | TFFALIEAAKFSKQWLPFCKKNNVQDRSPEVYFSSES |
| | | | HSRSDEAENLKMMYEDMKSRVEHVVESGKVETAFI |
| | | | TCDQFRGVFDLWTDKFSRHDHPTIIQVLQNSETDMD |
| | | | NTRKYIMPNLIYVSREKSKVSPHHFKAGALNTLLRVS |
| | | | GVMTNSPIILTLDCDMYSNDPATLVRALCYLTDPEIK |
| | | | SGLGYVQFPQKFLGISKNDIYACENKRLFIINMVGFD |
| | | | GLMGPTHVGTGCFFNRRAFYGPPYMLILPEINELKPY |
| | | | RIADKSIKAQDVLSLAHNVAGCIYEYNTNWGSKIGFR |
| | | | YGSLVEDYYTGFMLHCEGWRSVFCNPKKAAFYGDS |
| | | | PKCLVDLVGQQIRWAVGLFEMSFSKYSPITYGIKSLD |
| | | | LLMGLGYCNSPFKPFWSIPLTVYGLLPQLALISGVSV |
| | | | FPKASDPWFWLYIILFFGAYAQDLSDFLLEGGTYRK |
| | | | WWNDQRMLMIKGLSSFFFGFIEFILKTLNLSTPKFNV |
| | | | TSKANDDDEQRKRYEQEIFDFGTSSSMFLPLTTVAIV |
| | | | NLLAFVWGLYGILFCGGELYLELMLVSFAVVNCLPI |
| | | | YGAMVLRKDDGKLSKRTCFLAGNLHVGSYCVKLLR |
| | | | PQVTSPLRLIHNNNTSGWFKRKKHNMNESV |
| Spinach | cellulose | 70 | MEATGGMVAGSYKRNELVRIRHDSTDSGSKSLKNL |
| CESA1 | synthase A | | DGQICQICGDTVGVTSNGGVFVACNECAFPVCRPCY |
| | catalytic | | EYERKDGNQCCPQCKTRYKRQKGSLRVEGDDEEED |
| | subunit 1 | | VDDLDNEFNYERGTSKARHQWQGEDVDLSSSSRHG |
| | [UDP- | | SQPIPLLINGQVVSGEIPSATPDNQSVRSTSGPIGPEKR |
| | forming] | | GNHSLPYIDPCLPVPVRIVDPSKDLNSYGLGSVDWKE |
| | | | RVESWKLKQEKNMTHTGNRYSEGKGGDVEGSGSNG |
| | | | EELQLADDVRQPMSRIVPIPSSHLTPYRAVIIFRLIILVF |
| | | | FLQFRITHPVEDAYPLWLTSVICEIWFAMSWILDQFP |
| | | | KWYPINRETYLDRLAFRHDREGEPSQLAPIDVFVSTV |
| | | | DPLKEPPIITANTVLSILAVDYPVDKVSCYVSDDGSA |
| | | | MLTFEGLSETAEFARKWVPFCKKFSIEPRAPEFYFQQ |
| | | | KIDYLKDKIQPSFVKERRAMKREYEEFKVRINALVA |
| | | | KAQKVPEEGWTMQDGTAWPGNNPRDHPGMIQVFL |
| | | | GHSGGLDMDGNELPRLVYVSREKRPGFQHHKKAGA |
| | | | MNALIRVSAVLTNGAYILNVDCDHYFNNSKCLKEA |
| | | | MCFMMDPALGKKVCYVQFPQRFDGIDLHDRYANRN |
| | | | IVFFDINMKGQDGIQGPVYVGTGCCFNRQALYGYDP |
| | | | VLTEEDFEPNFIIKNCFGSRKKGKSGNKKYMDKKRG |
| | | | PKRSESSIPIFNMEDIEEGVEGYEDEKSLLMSQKRLEK |
| | | | RFGQSPVFIAATFMEMGGIPPTTNPATLLKEAIHVISC |
| | | | GYEDKSEWGKEIGWIYGSVTEDILTGFKMHARGWM |
| | | | SIYCMPPRPAFKGSAPLNLSDRLNQVLRWALGSIEIM |
| | | | LSRHCPIWYGYKGRLRFLERLAYINTVVYPLTSIPLIA |
| | | | YCILPAICLLTNKFIIPTLSNFASILFIMLFMSIAATGILE |
| | | | LRWSGVSIEDWWRNEQFWVIGGTSAHLFAVFQGLL |
| | | | KVLAGIDTNFTVTSKAADEDGDFAELYIFKWTALLIP |
| | | | PTTVLIVNLVGVVAGVSYAINSGYQSWGPLFGKLFFS |
| | | | FWVIAHLYPFLKGLLGRQNRTPTIVIVWSVLLASIFSL |
| | | | LWVRINPFTTDAEKAAAGNQCGINC |
| Spinach | cellulose | 71 | MMEDSQSGVKPTKQANEQVCQICSDNIGTTVDGEPF |
| CESA3 | synthase A | | VACDVCSFPVCRACYEYERKDGTQSCPQCKTRYKR |
| | catalytic | | QKGSPAIHGEKVEDSDVEDVVSDVNEPLGSSILKEKP |
| | subunit 3 | | QERMLGWHMNHGQSGELGPPTYDKEAPISHIPRLAT |
| | [UDP- | | GRTVSGDLSAASPGRFSMPSPGASTGANIRVSREFAS |
| | forming] | | PGFGNVAWKERIDGWKMKQEKSTGPPSVSHAPSEG |
| | | | RFANDIDASTEIAMDDPLLNDETRQPLSRKVPIPSSRI |
| | | | NPYRMVIVLRLAVLGIFLHYRVTNPVPNAYALWLIS |
| | | | VICEIWFAFSWILDQFPKWLPINRETYLDRLALRYDR |
| | | | EGEPSQLAAVDIFVSTVDPLKEPPLVTANTVLSILAVD |
| | | | YPVDKVSCYVSDDGAAMLTFEALSETSEFARKWVPF |
| | | | TKKYNIEPRAPEWYFSQKIDYLKDKVQTTFVKDRRA |
| | | | MKREYEEFKIRINGLVAKATKVPEEGWVMQDGTPW |
| | | | PGNNTRDHPGMIQVFLGQSGGLDTDGNELPRLVYVS |
| | | | REKRPGFTHHKKAGAMNSLVRVSAVLTNGPFMLNL |
| | | | DCDHYINNSKALREAMCFMMDPNLGKYCCYVQFPQ |
| | | | RFDGIDRNDRYANRNTVFFDINLRGLDGIQGPVYVG |
| | | | TGCVFNRTALYGYEPPIKPKPKKKGILSSCFGGSRKK |
| | | | SSKKDSKKKSKHADPTVPIFNLEDIEEGVEGTGFDDE |
| | | | KSLLMSQISLEKRFGKSEVFVASTLMENGGVPQSATP |
| | | | DTLLKEAIHVISCGYEDKTDWGAEIGWIYGSVTEDIL |
| | | | TGFKMHARGWRSIYCMPKLAAFKGSAPINLSDRLNQ |
| | | | VLRWALGSVEILFSRHCPLWYGYGGRLKWLERFAYI |
| | | | NTTIYPLTSIPLLAYCTLPAVCLLTGKFIIPQISNLASV |
| | | | WFLSLFLSIFATGILEMRWSGVGIDEWWRNEQFWVI |
| | | | GGISAHLFAVFQGLLKVLAGIDTNFTVTSKASDEDGD |
| | | | FTELYLFKWTTLLIPPTTILIVNLVAVVAGISYAINSGY |
| | | | QSWGPLFGKLFFAFWVIVHLYPFLKGLMGRQNRTPT |
| | | | IVVVWSILLASIFSLLWVRVDPFTTRVTGPDVHICGIN |
| | | | C |
| ATCSLA1_ | | 86 | ATGTCTCTATTTCTGAAGCCCTTCCTCTTCCTATAC |
| At4g16590 | | | GACACCACTCTTAGTCTTCTCTTACTTCTGTTCAAT |
| | | | GGATGGAGTCTTGAGGATACAGCAGCAGCCCAAA |
| | | | AGAGGCGTGAAGCAGACAAAAATGCTGCAGAAAC |
| | | | TGAATGGATCCAACTCCAATACTTGTGGACCAAAA |
| | | | CAAGGAGTGTTGTACTACTTCCCGTTTTCAAGGGT |
| | | | TTGGTGGTTATGTGTTTGGTTCTATCCATTATAGTG |
| | | | TTCTTCGAGAGTTTTTACATGAACTTTGTGATACTC |
| | | | TTCGTCAAGTTATTTAAACGTAAACCCCATAAAGT |
| | | | GTACAAATGGGAGGCCATGCAAGAAGATGTTGAG |
| | | | GTTGGACCCGATAACTACCCAATGGTTCTTATCCA |
| | | | AATACCAATGTACAATGAAAAAGAGGAAGGTGTG |
| | | | GACGTAGAGATTGCAAAATGGCAAAGCCAAGGCA |
| | | | TAAACATAAGGTGTGAAAGGAGAGATAACAGGAA |
| | | | CGGCTACAAAGCCGGAGCTATGAAAGAAGCTCTT |
| | | | ACGCAGAGCTACGTCAAGCAATGCGACTTCGTAGC |
| | | | AGTCTTCGATGCTGATTTCCAACCCGAGCCCGATT |
| | | | ATCTCATCCGCGCTGTCCCTTTCCTTGTCCACAACC |
| | | | CTGACGTTGCTCTAGTTCAAGCCCGATGGATATTT |
| | | | GTTAACGCGAACAAATGCTTGATGACGAGGATGC |
| | | | AAGAGATGTCTCTCAACTATCATTTCAAAGTGGAA |
| | | | CAAGAATCAGGGTCGACTAGACATGCTTTCTTCGG |
| | | | GTTTAATGGAACCGCGGGTGTATGGAGAATATCGG |
| | | | CAATGGAAGCAGCAGGAGGATGGAAATCAAGGAC |
| | | | CACAGTAGAGGACATGGACTTGGCTGTTCGTGTTG |
| | | | GTCTTCATGGCTGGAAATTTGTCTACCTTAACGAC |
| | | | CTCACGGTGAGAAACGAGCTTCCAAGCAAATTTAA |
| | | | GGCCTACAGATTCCAGCAACATAGGTGGTCCTGTG |
| | | | GACCGGCGAATCTATTTAGAAAAATGACGATGGA |
| | | | GATCATTTTCAATAAGAGAGTATCAATTTGGAAGA |
| | | | AGTTTTATGTGATCTACAGCTTTTTCTTCGTAAGGA |
| | | | AAGTGGCGGTACACTTCTTGACATTCTTCTTCTACT |
| | | | GTATAATTGTGCCAACAAGTGTCTTCTTCCCTGAA |
| | | | ATCCACATCCCATCTTGGTCTACCATTTACGTTCCC |
| | | | TCTTTGATCAGTATCTTCCACACCCTGGCAACTCCA |
| | | | AGATCCTTCTACCTCGTGATATTTTGGGTCTTGTTC |
| | | | GAGAATGTAATGGCTATGCATCGAACCAAAGGTA |
| | | | CGTGCATTGGCCTACTTGAAGGAGGAAGAGTAAA |
| | | | CGAATGGGTTGTGACCGAAAAACTAGGAGATGCTT |
| | | | TGAAGAGTAAGCTACTCTCTCGGGTAGTCCAAAGA |
| | | | AAATCTTGTTATCAAAGAGTGAATTCCAAGGAAGT |
| | | | GATGGTGGGGGTATACATATTAGGATGTGCACTCT |
| | | | ATGGCCTGATCTATGGGCACACATGGTTACATTTC |
| | | | TATCTTTTTCTTCAGGCCACAGCCTTTTTCGTCTCC |
| | | | GGTTTTGGTTTTGTCGGAACCTAA |
| ATCSLA1_ | | 87 | MSLFLKPFLFLYDTTLSLLLLLFNGWSLEDTAAAQKR |
| At4g16590 | | | READKNAAETEWIQLQYLWTKTRSVVLLPVFKGLV |
| | | | VMCLVLSIIVFFESFYMNFVILFVKLFKRKPHKVYKW |
| | | | EAMQEDVEVGPDNYPMVLIQIPMYNEKEEGVDVEIA |
| | | | KWQSQGINIRCERRDNRNGYKAGAMKEALTQSYVK |
| | | | QCDFVAVFDADFQPEPDYLIRAVPFLVHNPDVALVQ |
| | | | ARWIFVNANKCLMTRMQEMSLNYHFKVEQESGSTR |
| | | | HAFFGFNGTAGVWRISAMEAAGGWKSRTTVEDMDL |
| | | | AVRVGLHGWKFVYLNDLTVRNELPSKFKAYRFQQH |
| | | | RWSCGPANLFRKMTMEIIFNKRVSIWKKFYVIYSFFF |
| | | | VRKVAVHFLTFFFYCIIVPTSVFFPEIHIPSWSTIYVPSL |
| | | | ISIFHTLATPRSFYLVIFWVLFENVMAMHRTKGTCIGL |
| | | | LEGGRVNEWVVTEKLGDALKSKLLSRVVQRKSCYQ |
| | | | RVNSKEVMVGVYILGCALYGLIYGHTWLHFYLFLQA |
| | | | TAFFVSGFGFVGT |
| ATCSLB1_ | | 88 | ATGGCGGATTCAAGCTTTTCTCTTCCTCCTCTTTGT |
| AT2G32610 | | | GAAAGGATCTCATACACGAACTATTTTCTAAGAGC |
| | | | TGTATATCTCACGGTTCTAGGCCTTTTCTTTTCTCTT |
| | | | CTCTTGCACCGAATCCGACATACGAGCGAATACGA |
| | | | CAACGTTTGGCTCGTGGCTTTCTTTTGTGAATCTTG |
| | | | TTTCTTCTTGGTATGTCTGCTTATTACTTGCCTAAA |
| | | | ATGGAGTCCTGCTGATACTAAACCCTTTCCTGATA |
| | | | GACTTGATGAAAGGGTTCATGACCTTCCTTCGGTG |
| | | | GATATGTTCGTGCCCACAGCAGATCCGGTTCGAGA |
| | | | GCCACCGATTATGGTTGTGGACACCGTGCTTTCGC |
| | | | TGTTAGCTGTAAATTATCCGGCAAATAAACTAGCT |
| | | | TGTTATGTGTCGGACGATGGATGCTCACCTCTCAC |
| | | | TTATTTCTCTCTCAAGGAAGCTTCTAAGTTCGCCAA |
| | | | GATTTGGGTACCGTTCTGCAAAAAGTACAACACTA |
| | | | GAGTTAGAGCTCCTTCTAGATATTTTCTGAAACCT |
| | | | ATAAGCGTCGCAACAGAGGATTATGAATTCAATAG |
| | | | AGACTGGGAAAAGACGAAGAGGGAGTACGAGAA |
| | | | GTTGAGGCGGAAAGTGGAAGATGCCACCGGAGAT |
| | | | TCTCATATGTTGGATGTAGAAGATGATTTTGAAGC |
| | | | ATTCTCAAACACAAAACCAAATGATCATTCAACTC |
| | | | TAGTTAAGGTGGTATGGGAGAACAAGGGAGGTGT |
| | | | AGGAGACGAGAAAGAGATCCCTCATATCATATAC |
| | | | ATATCAAGAGAGAAAAGACCAAATTATGTTCATA |
| | | | ATCAAAAATGTGGAGCCATGAACTTTCTGGCAAGA |
| | | | GTGTCAGGGTTGATGACAAACGCACCATACATCTT |
| | | | GAACGTGGATTGCGACATGTATGCCAATGATGCAG |
| | | | ATGTAGTCCGACAAGCAATGTGTATACTTCTGCAA |
| | | | GAATCATTAAATATGAAACATTGTGCTTTTGTTCA |
| | | | ATTCCGTCAAGAATTCTATGATTCAAGCACCGAGC |
| | | | TAATAGTCGTCCTACAATCACATTTGGGACGAGGA |
| | | | ATCGCGGGAATCCAAGGACCGATATATATAGGATC |
| | | | AGGATGCGTCCACACGAGAAGAGTTATGTATGGTT |
| | | | TATCTCCAGACGATTTCGAAGTTGATGGAAGTCTT |
| | | | TCTTCAGTTGCTACAAGGGAGTTTTTGGTTAAGGA |
| | | | TAGTTTAGCGAGAAGATTTGGTAATTCTAAAGAGA |
| | | | TGATGAAATCAGTGGTTGATGCAATACAAAGAAAT |
| | | | CCAAATCCACAAAATATACTTACAAACTCCATAGA |
| | | | AGCGGCTCGAGAAGTGGGACATTGTCAGTACGAG |
| | | | TACCAAACCAGCTGGGGAAACACCATCGGCTGGTT |
| | | | ATATGATTCAGTGGCGGAAGATTTAAACACGAGTA |
| | | | TCGGAATACATTCGAGAGGTTGGACTAGCTCATAC |
| | | | ATTTCTCCGGATACACCTGCATTTCTTGGATCTATG |
| | | | CCGGCAGGAGTACCCGAGGCGTTACTCCAGCAGC |
| | | | GTCGATGGGCGACAGGATGGATCGAAATCCTTTTC |
| | | | AACAAGCAAAGTCCGTTGCGAGGATTGTTTAGCAA |
| | | | GAAAATAAGATTCCGACAACGATTAGCTTATCTTT |
| | | | GCATTATCACCTGTCTAAGGTCAATCCCTGAGCTT |
| | | | ATTTATTGTCTCCTTCCTGCTTATTGCCTACTCCAC |
| | | | AACTCTACCTTATTCCCCAAGGGACTTTATTTAGGC |
| | | | ATAACTGTCACACTTGTTGGGATACATTGTCTCTAT |
| | | | ACTCTATGGGAATTTATGAGCCTTGGTTATTCCGTA |
| | | | CAATCGTGGCTAGTCTCCCAATCAGTTTGGAGAAT |
| | | | AGTAGCCACTAGTAGTTGGTTATTTAGCATCTTTG |
| | | | ATATCACACTCAAGCTTCTTGGCATCTCGGAAACG |
| | | | GTGTTCATAATCACTAAAAAGACTGTGGCTGGGAC |
| | | | CAAGTCAGCATTAGGGTCTGGACCCTCTCAAGGAG |
| | | | AAGACGTTGGTCCAAACTCAGACTTGTTTAAATTT |
| | | | GAATTTGATGGCTCACTTTGTTTCTTGCCTGGCACA |
| | | | TTTATTGTGTTGGTGAATATAGCCGCTCTAGCTGTT |
| | | | TTTTCTGTGGGTCTACAACGGTCGAGTTACAGCCA |
| | | | TGAAGGAGGTGGTTCGGGTCTGGCAGAGGCTTGCG |
| | | | GATGTGTTTTGGTAATGATGTTGTTCCTTCCATTTC |
| | | | TAATGGGTTTGTTTAAGAAAGGAAAATATGGAACC |
| | | | CCATTGTCTACTCTCTCTATAGCTGGCTTTTTAGCA |
| | | | GTTTTATTTGTTGTTTTCTCTGTTTGA |
| ATCSLB1_ | | 89 | MADSSFSLPPLCERISYTNYFLRAVYLTVLGLFFSLLL |
| AT2G32610 | | | HRIRHTSEYDNVWLVAFFCESCFFLVCLLITCLKWSP |
| | | | ADTKPFPDRLDERVHDLPSVDMFVPTADPVREPPIM |
| | | | VVDTVLSLLAVNYPANKLACYVSDDGCSPLTYFSLK |
| | | | EASKFAKIWVPFCKKYNTRVRAPSRYFLKPISVATED |
| | | | YEFNRDWEKTKREYEKLRRKVEDATGDSHMLDVED |
| | | | DFEAFSNTKPNDHSTLVKVVWENKGGVGDEKEIPHII |
| | | | YISREKRPNYVHNQKCGAMNFLARVSGLMTNAPYIL |
| | | | NVDCDMYANDADVVRQAMCILLQESLNMKHCAFV |
| | | | QFRQEFYDSSTELIVVLQSHLGRGIAGIQGPIYIGSGC |
| | | | VHTRRVMYGLSPDDFEVDGSLSSVATREFLVKDSLA |
| | | | RRFGNSKEMMKSVVDAIQRNPNPQNILTNSIEAAREV |
| | | | GHCQYEYQTSWGNTIGWLYDSVAEDLNTSIGIHSRG |
| | | | WTSSYISPDTPAFLGSMPAGVPEALLQQRRWATGWI |
| | | | EILFNKQSPLRGLFSKKIRFRQRLAYLCIITCLRSIPELI |
| | | | YCLLPAYCLLHNSTLFPKGLYLGITVTLVGIHCLYTL |
| | | | WEFMSLGYSVQSWLVSQSVWRIVATSSWLFSIFDITL |
| | | | KLLGISETVFIITKKTVAGTKSALGSGPSQGEDVGPNS |
| | | | DLFKFEFDGSLCFLPGTFIVLVNIAALAVFSVGLQRSS |
| | | | YSHEGGGSGLAEACGCVLVMMLFLPFLMGLFKKGK |
| | | | YGTPLSTLSIAGFLAVLFVVFSV |
| ATCSLE1_ | | 90 | ATGGTAAACAAAGACGACCGGATTAGACCGGTTC |
| AT1G55850 | | | ATGAAGCCGACGGTGAACCGCTTTTTGAGACTAGG |
| | | | AGAAGAACCGGTAGAGTGATTGCGTACCGGTTTTT |
| | | | CTCAGCCTCGGTTTTCGTGTGTATCTGTTTGATTTG |
| | | | GTTCTACAGAATTGGTGAGATTGGTGATAACCGTA |
| | | | CCGTTTTAGATCGATTAATCTGGTTTGTTATGTTTA |
| | | | TTGTGGAGATTTGGTTCGGTTTATATTGGGTAGTCA |
| | | | CACAATCTTCCCGGTGGAATCCGGTTTGGCGATTT |
| | | | CCCTTCTCCGATAGACTCTCTCGGAGATACGGAAG |
| | | | CGACCTTCCGAGGCTCGACGTCTTCGTTTGCACGG |
| | | | CGGATCCGGTGATTGAGCCGCCGTTGTTGGTGGTA |
| | | | AACACAGTCTTATCTGTGACGGCTCTTGACTACCC |
| | | | ACCGGAGAAACTCGCCGTTTATCTCTCAGATGACG |
| | | | GTGGTTCTGAGCTGACGTTCTATGCTCTCACGGAG |
| | | | GCAGCTGAGTTTGCTAAAACTTGGGTTCCCTTCTG |
| | | | CAAGAAGTTCAACGTTGAGCCAACATCTCCCGCTG |
| | | | CTTACTTGTCTTCCAAGGCAAACTGTCTTGATTCTG |
| | | | CGGCTGAGGAGGTGGCTAAGCTGTATAGAGAAAT |
| | | | GGCGGCGAGGATTGAAACGGCGGCGAGACTGGGA |
| | | | CGAATACCGGAGGAGGCGCGGGTGAAGTACGGTG |
| | | | ACGGGTTTTCACAGTGGGATGCTGACGCTACTCGA |
| | | | AGAAACCATGGAACCATTCTTCAAGTTTTGGTAGA |
| | | | TGGAAGAGAAGGGAATACAATAGCAATACCAACG |
| | | | TTGGTGTATTTATCAAGAGAAAAGAGACCTCAACA |
| | | | TCATCATAACTTCAAGGCTGGAGCAATGAACGCAT |
| | | | TGCTGAGGGTTTCTTCGAAAATTACTTGTGGGAAA |
| | | | ATCATACTAAACTTGGACTGTGATATGTACGCAAA |
| | | | CAACTCAAAGTCAACACGCGACGCGCTCTGCATCC |
| | | | TCCTCGATGAGAAAGAGGGAAAAGAGATTGCTTTC |
| | | | GTGCAGTTTCCGCAGTGTTTTGACAATGTTACAAG |
| | | | AAATGATTTGTATGGAAGCATGATGCGAGTAGGA |
| | | | ATTGATGTGGAATTTCTTGGATTGGATGGAAATGG |
| | | | TGGTCCGTTATACATTGGAACTGGATGCTTTCACA |
| | | | GAAGAGATGTGATCTGTGGAAGAAAGTATGGAGA |
| | | | GGAAGAAGAAGAAGAAGAATCTGAGAGAATTCAC |
| | | | GAAAATTTAGAGCCTGAGATGATTAAGGCTCTCGC |
| | | | GAGCTGCACTTATGAGGAAAACACTCAATGGGGA |
| | | | AAGGAGATGGGTGTGAAATATGGTTGCCCGGTAG |
| | | | AGGATGTAATAACTGGTTTGACGATTCAGTGTCGC |
| | | | GGATGGAAATCAGCCTACCTGAACCCGGAAAAGC |
| | | | AAGCATTTCTCGGGGTAGCGCCGACCAATTTGCAT |
| | | | CAAATGCTAGTGCAGCAGAGGAGATGGTCAGAGG |
| | | | GAGACTTTCAGATTATGCTTTCGAAGTATAGTCCG |
| | | | GTTTGGTATGGAAAAGGAAAGATCAGTTTAGGACT |
| | | | GATACTTGGTTACTGTTGCTATTGTCTTTGGGCTCC |
| | | | ATCTTCACTACCTGTGCTCATTTACTCTGTTTTGAC |
| | | | TTCTCTCTGTCTCTTCAAAGGCATTCCTCTGTTTCC |
| | | | AAAGGTCTCGAGCTCGTGGTTTATTCCGTTTGGAT |
| | | | ACGTCACTGTTGCAGCTACCGCATATAGCCTAGCC |
| | | | GAGTTCTTGTGGTGCGGAGGGACGTTCCGTGGATG |
| | | | GTGGAACGAGCAAAGGATGTGGCTTTATAGAAGA |
| | | | ACAAGCTCGTTTCTTTTCGGATTTATGGACACGATT |
| | | | AAGAAGCTACTTGGAGTTTCTGAGTCTGCGTTTGT |
| | | | GATCACAGCAAAAGTAGCAGAAGAAGAAGCAGCA |
| | | | GAGAGATACAAGGAAGAGGTAATGGAGTTTGGAG |
| | | | TGGAGTCTCCCATGTTTCTCGTCCTCGGAACACTCG |
| | | | GTATGCTCAATCTCTTCTGCTTCGCCGCAGCGGTTG |
| | | | CGAGACTTGTTTCCGGAGACGGTGGAGATTTGAAA |
| | | | ACAATGGGGATGCAATTTGTGATAACAGGAGTACT |
| | | | AGTTGTCATAAACTGGCCTCTGTATAAAGGTATGT |
| | | | TGTTGAGGCAAGACAAAGGAAAGATGCCAATGAG |
| | | | CGTTACAGTTAAATCAGTTGTTTTAGCTTTATCTGC |
| | | | CTGTACCTGTTTAGCGTTTTTGTAA |
| ATCSLE1_ | | 91 | MVNKDDRIRPVHEADGEPLFETRRRTGRVIAYRFFSA |
| AT1G55850 | | | SVFVCICLIWFYRIGEIGDNRTVLDRLIWFVMFIVEIW |
| | | | FGLYWVVTQSSRWNPVWRFPFSDRLSRRYGSDLPRL |
| | | | DVFVCTADPVIEPPLLVVNTVLSVTALDYPPEKLAVY |
| | | | LSDDGGSELTFYALTEAAEFAKTWVPFCKKFNVEPT |
| | | | SPAAYLSSKANCLDSAAEEVAKLYREMAARIETAAR |
| | | | LGRIPEEARVKYGDGFSQWDADATRRNHGTILQVLV |
| | | | DGREGNTIAIPTLVYLSREKRPQHHHNFKAGAMNAL |
| | | | LRVSSKITCGKIILNLDCDMYANNSKSTRDALCILLDE |
| | | | KEGKEIAFVQFPQCFDNVTRNDLYGSMMRVGIDVEF |
| | | | LGLDGNGGPLYIGTGCFHRRDVICGRKYGEEEEEEES |
| | | | ERIHENLEPEMIKALASCTYEENTQWGKEMGVKYGC |
| | | | PVEDVITGLTIQCRGWKSAYLNPEKQAFLGVAPTNL |
| | | | HQMLVQQRRWSEGDFQIMLSKYSPVWYGKGKISLG |
| | | | LILGYCCYCLWAPSSLPVLIYSVLTSLCLFKGIPLFPK |
| | | | VSSSWFIPFGYVTVAATAYSLAEFLWCGGTFRGWW |
| | | | NEQRMWLYRRTSSFLFGFMDTIKKLLGVSESAFVITA |
| | | | KVAEEEAAERYKEEVMEFGVESPMFLVLGTLGMLN |
| | | | LFCFAAAVARLVSGDGGDLKTMGMQFVITGVLVVIN |
| | | | WPLYKGMLLRQDKGKMPMSVTVKSVVLALSACTCL |
| | | | AFL |
| ATCSLG1_ | | 92 | ATGGAGACTCATAGAAAGAACTCGGTCGTCGGCA |
| AT4G24010 | | | ACATCCTCCACACGTGTCATCCTTGCCGGCGCACC |
| | | | ATTCCATATAGAATCTACGCCATATTTCACACGTG |
| | | | TGGCATCATAGCTCTCATGTATCACCATGTACACT |
| | | | CACTTGTCACAGCAAACAACACTCTTATAACATGT |
| | | | CTTCTTCTCCTCTCCGATATTGTTCTCGCCTTCATGT |
| | | | GGGCAACCACAACTTCCCTCCGCTTAAACCCGGTT |
| | | | CATCGGACCGAGTGCCCGGAGAAATATGCAGCTA |
| | | | AACCGGAGGACTTTCCAAAGCTGGACGTGTTTATA |
| | | | TGCACGGCTGATCCGTACAAGGAGCCTCCAATGAT |
| | | | GGTTGTTAACACCGCTTTATCGGTGATGGCTTACG |
| | | | AGTATCCGTCAGATAAGATCTCGGTGTATGTATCG |
| | | | GACGATGGAGGATCGTCGTTGACTTTCTTTGCTCTT |
| | | | ATTGAAGCTGCTAAGTTCTCTAAGCAGTGGTTGCC |
| | | | CTTTTGCAAGAAGAATAATGTTCAAGATCGGTCTC |
| | | | CTGAAGTTTATTTCTCTTCAGAGTCACATTCTCGAA |
| | | | GTGATGAAGCTGAAAACCTTAAGATGATGTACGA |
| | | | AGACATGAAGAGTAGAGTAGAACATGTGGTGGAG |
| | | | AGTGGAAAAGTTGAAACTGCGTTTATCACATGCGA |
| | | | CCAATTTCGTGGGGTATTCGATTTGTGGACCGACA |
| | | | AATTCAGTCGTCATGACCATCCCACAATTATTCAG |
| | | | GTGTTGCAAAATAGCGAGACAGATATGGACAATA |
| | | | CCAGAAAATATATAATGCCAAACCTAATCTATGTT |
| | | | TCAAGAGAGAAGAGTAAAGTTTCACCACATCATTT |
| | | | CAAAGCTGGTGCTCTTAATACTTTGCTACGAGTAT |
| | | | CAGGGGTGATGACAAATTCACCGATCATTCTAACA |
| | | | CTAGACTGTGATATGTATTCGAACGACCCGGCAAC |
| | | | ACTGGTTCGTGCTTTGTGCTATTTAACAGATCCTGA |
| | | | AATCAAATCCGGTTTAGGATATGTGCAGTTTCCTC |
| | | | AGAAATTTCTAGGAATAAGCAAAAATGATATATAT |
| | | | GCTTGTGAAAACAAACGCCTCTTCATTATTAATAT |
| | | | GGTTGGGTTTGATGGTCTAATGGGTCCAACTCATG |
| | | | TGGGAACTGGTTGTTTCTTTAATCGACGAGCTTTCT |
| | | | ATGGACCTCCATATATGTTGATTTTACCGGAGATA |
| | | | AATGAACTAAAGCCTTATCGGATTGCGGATAAGTC |
| | | | TATCAAAGCCCAAGATGTTTTGTCATTAGCACACA |
| | | | ATGTAGCAGGATGTATCTATGAGTACAATACCAAT |
| | | | TGGGGATCCAAGATTGGATTCAGATATGGGTCATT |
| | | | AGTAGAAGACTACTACACAGGGTTTATGCTCCATT |
| | | | GTGAAGGATGGAGATCAGTATTTTGCAACCCAAAA |
| | | | AAAGCTGCATTTTATGGAGATTCCCCAAAGTGCCT |
| | | | AGTTGATCTTGTGGGTCAACAAATCCGTTGGGCAG |
| | | | TTGGGCTTTTCGAAATGTCCTTTTCAAAGTATAGCC |
| | | | CAATTACCTATGGAATCAAGTCACTGGACCTTTTA |
| | | | ATGGGTTTAGGTTATTGCAACTCTCCGTTTAAGCC |
| | | | ATTTTGGTCAATTCCTCTGACCGTCTATGGACTTTT |
| | | | ACCACAGCTTGCACTCATTTCTGGAGTTAGTGTCTT |
| | | | CCCCAAGGCATCTGATCCGTGGTTTTGGCTTTACAT |
| | | | CATTTTATTCTTTGGGGCTTATGCCCAAGATCTATC |
| | | | AGACTTTTTATTGGAAGGAGGAACTTATCGGAAAT |
| | | | GGTGGAACGATCAAAGAATGTTGATGATAAAAGG |
| | | | ACTCTCTTCATTCTTCTTTGGTTTTATAGAGTTCATT |
| | | | CTCAAAACCCTAAACCTCTCCACACCTAAGTTCAA |
| | | | CGTCACCAGTAAAGCCAATGATGATGACGAACAG |
| | | | AGGAAGCGGTACGAGCAAGAAATCTTTGATTTCGG |
| | | | AACCTCTTCGTCCATGTTCTTGCCCTTGACCACGGT |
| | | | TGCCATAGTGAATCTGCTTGCTTTTGTCTGGGGGCT |
| | | | TTATGGTATTCTCTTCTGCGGAGGAGAACTCTACCT |
| | | | TGAGCTGATGCTGGTGAGCTTCGCGGTGGTGAATT |
| | | | GCTTACCGATCTACGGGGCTATGGTGTTGAGGAAA |
| | | | GATGATGGAAAATTATCAAAAAGAACTTGTTTCTT |
| | | | AGCTGGGAACCTCCACGTTGGTTCTTATTGTGTCA |
| | | | AGTTACTTCGTCCTCAAGTAACTTCACCCCTTAGGT |
| | | | TAATTCACAACAATAATACGTCTGGCTGGTTCAAG |
| | | | CGGAAGAAACACAATATGAATGAATCTGTGTAA |

Three channels were also detected that could possibly form the passage of the triterpenoid aglycone into the enzyme active site and a channel that could be the exit path for the glucuronidated product (FIGS. 49A-49C). In cellulose synthase A (CESA) proteins, transmembrane domains (TMDs) form a pore that allows passing of polysaccharide chains through the membrane to the extracellular space. The out-of-membrane domain in CESAs holds the enzyme active site, as well as four motifs that are conserved among all such proteins and predicted to be involved in substrate and/or acceptor binding, i.e., DD, DCD, ED and QVLRW (L. Sethaphong et al., Tertiary model of a plant cellulose synthase, Proc. Natl. Acad. Sci. U.S.A. 18, 7512-7 (2013).) (FIGS. 48—red boxes). Alignment of amino acid sequences of CESA and SOAP5 from spinach showed that ED (in SOAP5: ES) and QVLRW (in SOAP5: QLIKW) motifs are not conserved in SOAP5 (FIG. 48).

To check if the presence of CESA type amino acid motifs can affect SOAP5 activity, three mutated version of SOAP5 were created, as follows: (i) serine 442 substituted by aspartic acid (S442D), (ii) lysine 483 swapped with arginine (K483R) and (iii) both substitutions (S442D/K483R). Expression of mutated proteins together with SOAP1-4 in N. benthamiana demonstrated that modification of these motifs in SOAP5 alters enzyme performance. Wild type SOAP5 converts the entire medicagenic acid pool to MA-3-GlcA (MA-3-GlcA:MA:ratio 0:1), SOAP5-S442D or SOAP5-K483R could still effectively glucuronidate MA (MA-3-GlcA: MA ratio 0.03:1 and 0.09:1, respectively), while SOAP5-S442D/K483R was only able to partially process MA (MA-3-GlcA: MA ratio 0.90:1) (FIGS. 50A and 50B). Our observations indicate that change of the neutral amino acid to a negatively charged one causes steric hindrance in the enzyme active site in addition to affecting binding of the anionic substrate (UDP-GlcA), thus simultaneously reducing the enzymes efficiency. Changes of the amino acids (S442D and K483R) in the active site during the enzymes evolution could have been crucial for accepting UDP-GlcA instead of UDP-Glc and resulted in neofunctionalization of CESA proteins.

Summary: It appears that differences in key amino acid residues of CESA result in neofunctionalization of the enzyme to a CSLG enzyme activity. Another feature of CSLG that is crucial for its new triterpenoid related function was localization to endoplasmic reticulum (ER).

### Example 25: Subcellular Localization of the Triterpenoid Saponin Biosynthetic Pathway

Objective: To investigate the subcellular localization of the triterpenoid saponin biosynthetic pathway.

Methods: See Materials and Methods above.

Results: The subcellular localization of the triterpenoid saponin biosynthetic pathway was studies. The ER membrane system is a pertinent environment for the production of specialized metabolites such as steroids and triterpenoids. Many enzymes involved in triterpenoid biosynthesis, including squalene synthase, squalene epoxidase, oxidosqualene cyclases and most of the known CYP450s are membrane bound and operate within the ER compartment (C. A. Hasemann et al., Structure and function of cytochromes P450: a comparative analysis of three crystal structures. Structure. 1, 41-62 (1995); K. B. Linscott, T. D. Niehaus, X. Zhuang, S. A. Bell, J. Chappell, Mapping a kingdom-specific functional domain of squalene synthase. BBA—Molecular and Cell Biology of Lipids. 1861, 1049-1057 (2016); M. Christen et al., Structural insights on cholesterol endosynthesis: Binding of squalene and 2,3-oxidosqualene to supernatant protein factor. Journal of Structural Biology. 190, 261-270 (2015); R. Thoma et al., Insight into steroid scaffold formation from the structure of human oxidosqualene cyclase, Nature. 7013, 118-22 (2004).).

Transient expression of SOAP5 fused to a fluorescent reporter (SOAP5:mRFP) together with a cellular compartment marker (ER marker in fusion with GFP) demonstrated its localization to the ER network (FIGS. 51A-51F). Previously it was shown that the microsomal fraction from germinating soybean seeds contains a protein able to glucuronidate soyasapogenol B, supporting transmembrane localization of SOAP5 (Y. Kurosawa, H. Takahara, M Shiraiwa, UDP-glucuronic acid:soyasapogenol glucuronosyltransferase involved in saponin biosynthesis in germinating soybean seeds. Planta. 215, 620-9 (2002)). Next, fluorescently tagged versions of SOAP1, 2, 3, 4 and 5 were expressed in various combinations and ER co-localization of all proteins examined was observed (FIGS. 35A-35F, FIG. 52A-52F). Detection of high quantities of MA-3-GlcA in leaves simultaneously expressing all fusion proteins demonstrated that the fluorescent signals observed originated from functional, and hence properly folded and subcellularly localized proteins (FIG. 53). Also observed using fluorescence resonance energy transfer (FRET) was that SOAP1-5 proteins are located in proximity to each other and therefore possibly interact (FIGS. 54A-54B). Accurate colocalization of studied proteins could explain the high efficiency of performed reactions by decreasing the diffusion of the intermediates.

Summary: These studies showed that at least enzymes SOAP1-5 are localized in the ER in close proximity to each other.

General Conclusions from the Examples

These studies on steroidal alkaloid, steroidal saponin, and triterpenoid saponin biosynthesis in tomato, potato, eggplant, and spinach resulted in discovery and characterization of genes that complete the biosynthetic pathway of complex triterpenoid saponins and elucidate the biosynthetic pathways of complex steroidal alkaloids and steroidal saponins. Discovery of the unprecedented function of cellulose synthase like G proteins as glucuronosyltransferases of triterpenoid aglycones in seven plant species belonging to two distinct orders, proves that glycosylation of specialized metabolites is not exclusively performed by GTs belonging to 1 family Carbohydrate-Active enZYmes Database (CAZY). This finding will most likely trigger the discovery of additional functions of CSL enzymes related to other specialized metabolites (SMs). Additionally, the discovery of unique SOAP6 fucosyltransferase activity was demonstrated, as well as characterization of first BAHD acyltransferase (SOAP10) capable of acetylating sugar moieties of triterpenoid saponins. Similarly, GAME15 may in certain embodiments glycosylate steroidal alkaloids and steroidal saponins. In some embodiments, GAME15 glycosylation comprises transfer of a glucuronic acid (glucuronosyltransferase activity). Moreover, decreased expression of steroidal alkaloids and steroidal saponins provides a new approach to the increase in content of plant cholesterols, phytocholesterols, cholestenols, phytocholestenols, and phytosterols, which are useful as nutritional, cosmetic, and pharmaceutical agents,

The unexpected finding of CSLG proteins activity as triterpenoid glucuronosyltransferases shows that glycosylation of SMs is not exclusively executed by family 1 type UGTs. Hence, this report is likely to trigger the discovery of CSL enzymes functioning in modification of other classes of terpenoids and SMs, as well as in other classes of steroidal alkaloids and/or steroidal saponins. It moreover provides a fundamental example of how enzyme activity in one of the few principal plant processes, i.e., the cellulose synthesis machinery constructing cell walls, is ‘hijacked’ in order to produce a set of unrelated, defense specialized metabolites. Co-localization of SOAP5 at the spinach ER membrane with most other pathway proteins rather than the cytosol (in which UGTs localize) was part of evolving its new function. It suggests the importance of physical proximity between these enzymes for efficient triterpenoids production. This physical proximity was also reflected at the gene level as well, with respect to the GAME genes as a gene cluster in tomato. Apart from glucuronic acid, a yet undisclosed fucosyltransferase was identified acting on metabolites (i.e. SOAP6), as all plant fucosyltransferases to date were associated with fucosylation of cell wall polysaccharides and glycoproteins. While BAHD type acyltransferases are frequently associated with modification of SMs they were on no occasion reported to acylate triterpenoids as is demonstrated here for SOAP10 acetylating the C-28 fucose moiety of spinach triterpenoid saponins. With similarity to glucuronidation, the acylated fucose domain in triterpenoid saponins is important for their efficacy as therapeutic agents, e.g., adjuvanticity of the renowned QS-21, a potent saponin vaccine adjuvant from Quillaja saponaria.

This work delivers unparalleled strategy for heterologous engineering plants or yeast or other eukaryotic cell systems for sustainable production of high-value metabolites (e.g. glycyrrhizin, soyasaponins, QS-21 adjuvant) for food and pharmaceutical industries by filling a gap in biosynthetic pathways of many glucuronide-type triterpenoid saponins. In addition, it supports engineering endogenous genes within a plant triterpenoid saponin biosynthetic pathway to either increase beneficial triterpenoid saponins, for example but not limited to glycyrrhizin in Chinese licorice, or decrease bitter or unwanted triterpenoid saponins, for example but not limited to their reduction in quinoa. Likewise, toxins and bitter tasting compounds are decreased by the reduction of unwanted steroidal alkaloids and/or steroidal saponins.

While certain features of genetically modified cells and uses thereof for producing steroidal alkaloids, steroidal saponins, and triterpenoid saponins have been illustrated and described herein, many modifications, substitutions, changes, and equivalents will now occur to those of ordinary skill in the art. The foregoing description of the specific embodiments will so fully reveal the general nature of the invention that others can, by applying current knowledge, readily modify and/or adapt for various applications such specific embodiments without undue experimentation and without departing from the generic concept, and, therefore, such adaptations and modifications should and are intended to be comprehended within the meaning and range of equivalents of the disclosed embodiments. It is to be understood that the phraseology or terminology employed herein is for the purpose of description and not of limitation. The means, materials, and steps for carrying out various disclosed functions may take a variety of alternative forms without departing from the invention. It is, therefore, to be understood that the appended claims are intended to cover all such modifications and changes as fall within the true spirit of the disclosure herein.

### Example 26: Reducing the Expression Level of Yellow Pea GAME 15 (Cellulose SYnmthase Like (CSYL)) Using CRISPR CAS9 For Reducing Pea Saponins

Objective: To generate a plant with reduced expression of the Yellow Pea GAME 15 (CSyL) gene and reduced saponins, guides were designed against the 1^{st} gene exon of the target gene as shown in FIG. 55. The guide RNAs were cloned together with the CAS9, and the resulting vector is shown in FIG. 56.

### Methods:

Yellow Pea (Pisum sativum) plants were edited through transformation of embryonic cotyledons with a disarmed Agrobacterium tumefaciens strain, EHA105 according to the method of Schroeder H. E. et al. (1993) “Transformation and Regeneration of Two Cultivars of Pea (Pisum sativum L.)” Plant Physiol. 1993, 101(3), pp. 751-757, with some minor modifications. Bacteria was harboring a plasmid with a T-DNA fragment that consists of the elements as detailed in Table 20. The expression cassettes, compiled from these elements, are represented in FIG. 56.

TABLE 20
DNA elements present in T-DNA used to generate the edited Pea plants.

| # | Element | Function | Source of sequence |
|---|---|---|---|
| 1 | Left Border | Marks the left border of T-DNA region | Agrobacterium |
| | (LB) | which will transfer to plant genome | tumefaciens |
| 2 | [NOS | To drive the expression of the selection | Agrobacterium |
| | promoter] | marker, for kanamycin resistance | tumefaciens |
| 3 | [BlpR] | Phosphinotrycin (a.k.a Bialaphos or PPT); | Streptomyces |
| | | Herbicide resistance to select transformed | hygroscopicus |
| | | plants in the first generation | |
| 4 | [NOS | To terminate transcription of selection and | Agrobacterium |
| | terminator] | reporter marker genes | Tumefaciens |
| 5 | [35S | To drive the expression of a reporter gene | [Cauliflower mosaic virus] |
| | promoter] | | |
| 6 | [DsRed2] | Fluorescent marker to identify transgenic | [Discosoma coral] |
| | | regenerating plant | |
| 7 | [Ubiquitin | To drive the expression of the [CAS9] | [Petroselinum crispum] |
| | promoter | gene | |
| | (PcUbq)] | | |
| 8 | [AtCAS9] | To facilitate gene editing in the cell | [Codon-optimized for |
| | | | Arabidopsis from |
| | | | Streptococcus pyogenes |
| | | | sequence] |
| 9 | [rbcS-3a | To terminate transcription of [AtCAS9] | [Pisum sativum] |
| | terminator] | gene | |
| 10 | [AtU6-26 | To drive the expression of the single- | [Arabidopsis thaliana] |
| | promoter] | guided RNA (sgRNA) | |
| 11 | [gRNA | Portion of sgRNA which helps in making | [Streptococcus pyogenes] |
| | scaffold] | its secondary structure | |
| 12 | [CSLG | Portion of sgRNA that drives the gene | [Pisum Sativum (from |
| | gRNA A; | editing specifically to the [CSLG] locus | CSLG gene)] |
| | CSLG gRNA | | |
| | B; CSLG | | |
| | gRNA C] | | |
| 13 | [Poly A | To terminate transcription of sgRNAs | Synthetic |
| | (7XA)] | | |
| 14 | Right Border | Marks the left border of T-DNA region | Agrobacterium |
| | (RB) | which will transfer to plant genome | tumefaciens |

To generate plants with reduced expression of the Yellow Pea CSLG (Psat1g004960.1; PulseDB—https://www.pulsedb.org/bio_data/1034700) and consequently reduced saponins, the CRISPR/CAS9 method of genome editing was used. Three (3) guide RNAs were designed to target the 1st gene exon (see the target gene scheme in FIG. 55). The guide RNAs were cloned together with the CAS9, and the other vector elements as shown in FIG. 56 and described in detail in Table 20. The targeted DNA-breaks, in the CSLG locus, carried out by CAS9 activity and the sgRNA, are repaired through the NHEJ DNA-repair mechanism, which is error-prone. The repair errors result in mutated versions of the CSLG, usually reducing its expression in all plant tissues, including seeds.

### Results:

Editing Results (1^{st} Generation). Pea plant #169-2 was cut into three parts (named a-c), all rooted on Kanamycin-containing media. A single tendril from each plant was sampled and used for genomic DNA extraction, followed by PCR to amplify the targeted region of the CSyL gene. The PCR showed two comparable intensity bands: An expected size band of 525 bp and a shorter band of approx. 70 bp shorter (see gel image in FIG. 57, compared to WT Pea CSyl PCR product).

The mixture of PCR products from plant #169-2a was sent to Sanger sequencing. The sequence output was aligned to wild type sequence of CSLG (upper sequence) using “SnapGene” software. FIG. 58 shows the “editing window” in the CSLG Exon 1, i.e., the area in which the 3 guide RNAs were designed to target. The Exon 1 fragment is in light grey. The 3 CSLG guides are in dark grey. The sequence alignment shows clear editing of the targeted area in Exon 1 at two separate places marked in light grey with internal dashes: a 6 bp deletion in the first (Guide A) target and a larger 63 bp deletion in the second (Guide B) target (see FIG. 58).

Editing Results (2^{nd} Generation). T2 seeds of Pea plant #169-2A were germinated. The target gene was amplified and sequenced in selected T2 plants. The PCR products from T2 plants #169-2a-18, #169-2a-20, #169-2a-28 were sent to Sanger sequencing. The sequences output were aligned to wild type sequence of CSLG (upper sequence) using “SnapGene” software. FIG. 59 shows the “editing window” in CSLG Exon 1, meaning—the area in which the 3 guides were designed to target. The Exon 1 fragment is in light grey. The 3 CSLG RNA guides are in dark grey. In all three samples checked, precise inheritance of the mutations from T1 mother plant was detected: −6 bp and −63 bp denoted by light grey with internal dashes. All checked progenies were homozygous for those mutations (see checked progeny #18, 20, and 28 in FIG. 59).

Saponion Level Characterization in Pea Line with Reduced GAME 15 (CSLG) Expression

Pea grains derived from the homozygous pea mutant and non-edited (WT) underwent metabolic analysis for the two major pea saponins (Saponin Bb (soyasaponin 1; Compound 36) and Saponin βg; soyasaponin VI; Compound 35). Their chemical structures are shown in FIGS. 60A and 60B. Their identification in non-edited plant (WT) is shown in FIGS. 61A-61B (Saponin βg) and FIGS. 61C-61D (Saponin Bb), and their reduction by 98-99% is shown in FIG. 62 compared with an un-modified cell.

The coding region for pea GAME15 (CSLG) is set forth in SEQ ID NO: 125 (Pisum sativum cellulose synthase-like protein G1 (LOC127135727), mRNA):

(SEQ ID NO: 125)
ATGGCAACCTTCACACTTCACAAAGAAACAGTTCAATCATGGTTACTT
CTAAGTAGAACTCACATATTATTCCACTTCATATGTGTCTTGTTTCTC
TTTTATTATCGTATCAAAAATTTCATTGTTTCATATCCATGGATTTTA
ATGACACTAGCTGAAACTATTCTATCAGTTATGTGGTTTTTCAACCAA
GCGTACCGGTGGCGGCCGGTGAGTCGTTCGGTTATGACCGAGAAACTC
CCGGCCGACCAGAATTTACCGGGACTCGACATATTTGTGTGTACGATC
GATCCTGAGAAAGAACCAACCGTCGAAGTTATGAACACGGTTGTTTCG
GCTGTTGCTATGGATTACCCTAGTGATAAACTTTGTGTTTATCTTTCT
GATGATGGTGGTTCTCCTGTCACTTTATATGGAATTAAAGAGGCTTCT
CAATTTGCTAAAGCTTGGGTTCCTTTTTGTAAAAAATATGGTGTTAAA
TCAAGGTGCCCTAAGGTTTTTTTCTCTCCTTTGGGTGAGGATGAACAT
GTTCTTAGGTCACATGAATTTGAAGTAGAAAGAGACCAGATTAAGGTG
AAGTATGAGAAAATGCAGAAAAATATTGAGAAATTCAGTTCTGATCCA
AAGAATCTTTGTATGGTTAATGACAGAGCTTCTCGGATTGAGATTATA
AATGAGGAATCAGAAATTCCACGTGTGATTTATGTGTCTCGTGAAAGA
AGACCATCACTCCCTCACAAGTTCAAAGGAGGTGCCCTCAATACATTG
CTTAGAGTGTCAGGTTTGATAAGTAATGGGCCTTATGTACTTGCAGTG
GACTGTGATATGTATTGCAATGATCCATCATCAGCCAAACAAGCAATG
TGTTTCTTTCTTGATCCAGAAACCTCTAAATATATTGCTTTTGTTCAA
TTCCCTCAAATGTTTCACAATCTTAGCAAAAAGGACATCTATGATAAC
CAATCCAGAACTGCTTTTAAGACAATGTGGCAAGGGATGGATGGATTG
AGAGGTCCAGGTCTTTCTGGAAGTGGTAATTACTTGAATAGAAGTGCA
TTATTATTTGGAAGTCCAAATCAAAAAGATGAGTATCTTCATGATGCC
AAAAACTACTTTGGAGGATCAACCACATACATAGAATCACTCAAGGCC
ATTCGCGGACAACAAGTTATCAAAAAGAATATTTCCAAAGAAGAAATT
TTGCAAGAAGCTCAAGTAGTTTCATCTTGCTCCTATGAATCAAACACA
AAATGGGGCACAGAAATAGGATTCTCATACGGAATTTTACTAGAGAGT
ACTATCACTGGCTATCTTTTACATTGTAGAGGATGGAAATCAGCATAC
CTTTATCCAAAAACACCTTGTTTCTTAGGGTGTGCACCAACTGATATC
AAAGAAGGAATGCTTCAATTAGTGAAATGGTTGTCTGAACTTTGCTTG
CTTGCTATCTCTAAATATAGTCCTTTTACTTATGGATTTTCAAGAATG
TCAACTATTCATAACTTCACTTATTGCTTCATGTCAATTTCATCTATA
TATGCTATTGGGTTCATCCTTTATGGTATTGTGCCTCAAGTTTGCTTC
TTCAAAGGAATTCCTGTATTTCCAAAGGTCACAGATCCTTGGTTTGTA
GTGTTTGCAATACTATACATAGCCACACAAATTCAACATTTAATAGAG
GTAATTTCTGGCGATGGCTCAGTTTCAATGTGGTGGGACGAACAAAGA
ATCTGGATTCTAAAATCAGTTACAAGTTTATTTGCAATGATAGAGGCA
GTTAAGAAATGGTTAGGATTGAACAAGAAAAAATTCAACTTGTCAAAC
AAAGCAATTGATACAGACAAAGAAAAAATCAAGAAATATGAACAAGGT
AGGTTTGATTTTCAAGGTGCGGCTTTGTATATGTCTCCAATGATTGTG
TTGCTCATTGTCAACACTATTTGTTTCTTTGGTGGTTTATGGAGGCTT
TTCAAAATAAGAGATTTTGAAGATATGTTTGGCCAACTTTTCTTAGTA
AGTTACGTCATGGCTCTTAGTTATCCAATTTTTGAAGGGATTTTAACT
ATGAAAAGTAAGAGTGGATAG

The amino acid sequence of cellulose synthase-like protein G1 ([Pisum sativum] Sequence ID: XP_050918530.1Length: 694) is set forth in SEQ ID NO: 126.

(SEQ ID NO: 126)
MATFTLHKETVQSWLLLSRTHILFHFICVLFLFYYRIKNFIVSYPWIL
MTLAETILSVMWFFNQAYRWRPVSRSVMTEKLPADQNLPGLDIFVCTI
DPEKEPTVEVMNTVVSAVAMDYPSDKLCVYLSDDGGSPVTLYGIKEAS
QFAKAWVPFCKKYGVKSRCPKVFFSPLGEDEHVLRSHEFEVERDQIKV
KYEKMQKNIEKFSSDPKNLCMVNDRASRIEIINEESEIPRVIYVSRER
RPSLPHKFKGGALNTLLRVSGLISNGPYVLAVDCDMYCNDPSSAKQAM
CFFLDPETSKYIAFVQFPQMFHNLSKKDIYDNQSRTAFKTMWQGMDGL
RGPGLSGSGNYLNRSALLFGSPNQKDEYLHDAKNYFGGSTTYIESLKA
IRGQQVIKKNISKEEILQEAQVVSSCSYESNTKWGTEIGFSYGILLES
TITGYLLHCRGWKSAYLYPKTPCFLGCAPTDIKEGMLQLVKWLSELCL
LAISKYSPFTYGFSRMSTIHNFTYCFMSISSIYAIGFILYGIVPQVCF
FKGIPVFPKVTDPWFVVFAILYIATQIQHLIEVISGDGSVSMWWDEQR
IWILKSVTSLFAMIEAVKKWLGLNKKKFNLSNKAIDTDKEKIKKYEQG
RFDFQGAALYMSPMIVLLIVNTICFFGGLWRLFKIRDFEDMFGQLFLV
SYVMALSYPIFEGILTMKSKSG*

The genomic region for the gene sequence for Pisum sativum cellulose synthase-like protein G1 is set forth in SEQ ID NO: 127.

(SEQ ID NO: 127)
caaataaattaggataataatataatttcttattaaacgcgtggacat
atatatatatataggaatagacatatattagtaaagaaactaactaat
tcctataaaaaagaaaagaaaaaacctaattattttattaatctaaac
acaacaattatctcaacaacaaaaaaaaatttaaacataattatgagg
atttcattttaagatacaaatacttatgcactttgaattcttgaagaa
gttccactatccactcttacttttcatagttaaaatcccttcaaaaat
tggataactaagagccatgacgtaacttactaagaaaagttggccaaa
catatcttcaaaatctcttattttgaaaagcctccataaaccaccaaa
gaaacaaatagtgttgacaatgagcaacacaatcattggagacatata
caaagccgcaccttgaaaatcaaacctaccttgttcatatttcttgat
tttttctttgtctgtatcaattgctttgtttgacaagttgaatttttt
cttgttcaatcctaaccatttcttaactgcctctatcattgcaaataa
acttgtaactgattttagaatccagattctttgttcgtcccaccacat
tgaaactgagccatcgccagaaattacctctattaaatgttgaatttg
tgtggctatgtatagtattgcaaacactacaaaccaaggatctgtgac
ctgaagtatcaatcattgaaaatatgttaaattattataaaatatatt
acaaaaatagaattagtgttaaagaaatgtggttgatcctaactcatc
tatgcaaaataggtttgtaaggtgagaatctctcctacttataaacat
gttcaaaatatatattattcgatgtgagactcttaaaatatctatcat
gacaaagactgaatatatggaaggtggaaataaatggttggtggttcg
atagtagaaacctaatagtaggtggctcaacaaatcttaaatgttgtt
agacttaactcatccctacgaaactagctagtagagttaggatcgtgc
cacttataagtacatgttcaaacatgtaacacttttaacaactagtaa
aaagaattgagttggagagctttactacctttggaaatacaggaattc
ctttgaagaagcaaacttgaggcacaataccataaaggatgaacccaa
tagcatatatagatgaaattgacatgaagcaataagtgaagttatgaa
tagttgacattcttgaaaatccataagtaaaaggactatatttagaga
tagcaagcaagcaaagttcagacaaccatttcactaattgaagcattc
cttctttgatatcagttggtgcacaccctaagaaacaaggtgtttttg
gataaaggtatgctgatttccatcctctacaatgtaaaagatagccag
tgatagtactctctagtaaaattccgtatgagaatcctatttctgtgc
cccattttgtgtttgattcataggagcaagatgaaactacttgagctt
cttgcaaaatttcttctttggaaatattctttttgataacttgttgtc
cgcgaatggccttgagtgattctatgtatgtggttgatcctccaaagt
agtttttggcatcatgaagatactcatctacaatcatgaacagaagaa
atcataagaaaaaatctccaataaaaacctttttgtaatacataatga
ggcaatacgtacctttttgatttggacttccaaataataatgcacttc
tattcaagtaattaccacttccagaaagacctggacctctcaatccat
ccatcccttgccacattgtctaaaataaaatttgcaatatgtcatcat
tgcataataaacaaaatcggatttcataccgtcaatctttgttgtatt
cacgctgtcattgttttattaaaagttcgatcttaattggacgatcat
gatcaaatgacaaaaacactatataaggactaactgcaaattgcagtc
ccttgaaaaaaatgaaaaatctaaatgaagaacttcattattattttt
tatttaccttaaaagcagttctggattggttatcatagatgtcctttt
tgctaagattgtgaaacatttgagggaattgaacaaaagcaatatatt
tagaggtttctggatcaagaaagaaacacattgcttgtttggctgatg
atggatcattgcaatacatatcacagtccactgcaagtacataaggcc
cattacttatcaaacctgacactctaagctgcaaacacaaacaaaaat
atgttatattttattaactgcatcacaaattaaattttaatatgatat
acacaaaatacacaccaatgtattgagggcacctcctttgaacttgtg
agggagtgatggtcttctttcacgagacacataaatcacacgtggaat
ttctgattcctcatttataatctaaaataataaatataaagtaatatt
aaaaaaatatttttattttattttttactgttaataaataataaattt
tgattttttaattatttaaaaaataacttcttcaacacactcaaataa
acaccaaaatataacaactaaataattgtagaaatatgaaatacactt
tatttatttatttatttatttgttgttaatttattattgtattcgatc
taaaacattttattatatatactatatatatatatagtatatatatta
tatatatatatataatatatatatatatatatatttatatattattct
accaaattagattgtagtttttgaatttgtggtttaatataattaagt
gacaaataactattgcacgacagtgtcaaaataaatgcatcacaatta
aaccttataaataatttaaattaattttaaaatagatatgacttattt
aaataagtctgatccaacttaaatattttaacttaatttatattaatc
ataacatatttgtttgtttttctgtgtataaattgagtattatgttct
atatttttaacaattttttatttttttattttatcaaataattatact
atatatatatataatatatatatatatatatatatattatatatatat
atatatatatataatatatatatatatgataatatatatataatttat
ctaaaaaattaaaatttaattataatataaaaaagagagagtattctc
ttaaaatataggaatgcaatgttttcaagttgaaattgaatcaaaact
tttgtcaaactatttaaatataaatttggtgaattgaaaaatatttgt
taaaagattgagtttgaaaacttaaaacctttaattaatatatttgat
aatatttatcttgttttttctattagtgaaatatatttagactgcttt
tatacctaattaactagtgaaaaaaacaaataaaccttactaactgta
aatatttacacataaaaacatttatttattttccgttaactagttaac
tagtaattattccatcaaattgattattgatcataatccaactttagt
ttttattagtcatacctcaatccgagaagctctgtcattaaccataca
aagattctttggatcagaactgaatttctcaatatttttctgcatttt
ctcatacttcacctgtcaaattgttaaaacatggtatcaatacaaatt
atgttaactaattttatatatatatcagctatataaaccatataaaat
accttaatctggtctctttctacttcaaattcatgtgacctaagaaca
tgttcatcctcacccaaaggagagaaaaaaaccttagggcaccttgat
ttaacaccatattttttacaaaaaggaacccaagctttagcaaattga
gaagcctctttaattccatataaagtgacaggagaaccaccatcatca
gaaagataaacacaaagtttatcactagggtaatccatagcaacagcc
gaaacaaccgtgttcataacttcgacggttggttctttctcaggatcg
atcgtacacacaaatatgtcgagtcccggtaaattctggtcggccggg
agtttctcggtcataaccgaacgactcaccggccgccaccggtacgct
tggttgaaaaaccacataactgatagaatagtttcagctagtgtcatt
aaaatccatggatatgaaacaatgaaatttttgatacgataataaaag
agaaacaagacacatatgaagtggaataatatgtgagttctacttaga
agtaaccatgattgaactgtttctttgtgaagtgtgaaggttgccata
acaagtcaatacttgaatatttttcacaacaaaattaagttgcgtatg
gtttataataggtgaagtttcatttatatataatgtatctatcacttg
ttagttgttgaagcaaaataaataaataaattctatagtcattagaaa
tgaaaatcaaatactaattttaaagtaactctcgtggaactcgc

The sequences for the guide sequences against Exon 1 are set forth in Guide A, Guide B, and Guide C (FIG. 56). Sequence for Guide A: ccggccgccaccggtacgctTGG (SEQ ID NO:128); Sequence for Guide B: gagtcccggtaaattctggtCGG (SEQ ID NO: 129); Sequence for Guide C: taccgtcgaagttatgaacaCGG (SEQ ID NO: 130)

LENGTHY TABLES
The patent application contains a lengthy table section. A copy of the table is available in electronic form from the USPTO web site (https://seqdata.uspto.gov/?pageRequest=docDetail&DocID=US20240315192A1). An electronic copy of the table will also be available from the USPTO upon request and payment of the fee set forth in 37 CFR 1.19(b)(3).

What is claimed is:

1. A genetically modified plant cell having decreased expression of a cellulose synthase like G (CSLG) gene encoding a CSLG enzyme compared to a corresponding unmodified cell, wherein when compared to a corresponding unmodified cell, said genetically modified cell comprises a decreased content of (i) at least one steroidal alkaloid, a derivative thereof, a metabolite thereof, or a biosynthetic intermediate thereof; or (ii) at least one steroidal saponin, a derivative thereof, a metabolite thereof, or a biosynthetic intermediate thereof; or (iii) at least one triterpenoid saponin, a derivative thereof, a metabolite thereof, or a biosynthetic intermediate thereof, wherein said CSLG enzyme has the amino acid sequence set forth in any one of SEQ ID NOs:126, 31, 33, 35, 37, 39, 41, 66, 81, 94, 96, 98, 100, 102, or 104; or a homolog thereof having at least 90% identity to and at least 90% coverage of the amino acid sequence set forth in any one of SEQ ID NOs:126, 31, 33, 35, 37, 39, 41, 66, 81, 94, 96, 98, 100, 102, or 104.

2. The genetically modified plant cell according to claim 1, wherein said CSLG gene has the nucleic acid sequence set forth in any one of SEQ ID NOs:125, 127, 30, 32, 34, 36, 38, 40, 65, 80, 93, 95, 97, 99, 101, 103, or 105; or a homolog thereof having at least 90% identity to and at least 90% coverage of the nucleic acid sequence set forth in any one of SEQ ID NOs:125, 127, 30, 32, 34, 36, 38, 40, 65, 80, 93, 95, 97, 99, 101, 103, or 105.

3. The genetically modified plant cell according claim 1, wherein said triterpenoid saponin or said derivative, metabolite, or biosynthetic intermediate thereof, is a bitter tasting compound, a toxic compound, or a compound having hormone mimicking properties, or any combination thereof.

4. The genetically modified plant cell according to claim 1,
(a) wherein said steroidal glycoalkaloid is selected from an esculeoside, a dehydroesculeoside, alpha-tomatine, dehydrotomatine, alpha-chaconine, alpha-solanine, alpha-solasonine, and alpha-solmargine, or any combination thereof, or
(b) wherein said steroidal saponin is an uttroside B, a tomatoside, or any combination thereof, or
(c) wherein said triterpenoid saponin is selected from medicagenic acid 3-O-glucuronide (MA-3-GlcA), 3-O-β-D-glucuronopyranosyl-28-O-β-D-fucopyranosyl-medicagenic acid, 3-O-[β-D-glucuronopyranosyl]-28-O-[α-L-rhamnopyranosyl-(1->2)-ρ-D-fucopyranosyl]-medicagenic acid, 3-O-[β-D-glucuronopyranosyl]-28-O-[β-D-glucopyranosyl-(1->4)-α-L-rhamnopyranosyl-(1->2)-β-D-fucopyranosyl]-medicagenic acid, 3-O-[β-D-xylopyranosyl-(1->3)-β-D-glucuronopyranosyl]-28-O-[β-D-glucopyranosyl-(1->4)-a-L-rhamnopyranosyl-(1->2)-β-D-fucopyranosyl]-medicagenic acid, 3-O-[β-D-xylopyranosyl-(1->3)-β-D-glucuronopyranosyl]-28-O-[β-D-glucopyranosyl-(1->4)-α-L-rhamnopyranosyl-(1->2)-4-acetyl-β-D-fucopyranosyl]-medicagenic acid, glycyrrhetinic acid 3-O-monoglucuronide, glycyrrhizin, hederagenin-3GlcA, gypsogenin-3GlcA, gypsogenic acid-3GlcA, oleanolic acid-3GlcA, or augustic acid-3GlcA, soyasaponin I, soyasaponin VI, and a QS-21 adjuvant or any combination thereof; or
(d) a combination thereof.

5. The genetically modified plant cell according to claim 1, wherein when compared to a corresponding unmodified cell, said genetically modified cell further comprises an increased content of (i) at least one steroidal alkaloid, a derivative thereof, a metabolite thereof, or a biosynthetic intermediate thereof; or (ii) at least one steroidal saponin, a derivative thereof, a metabolite thereof, or a biosynthetic intermediate thereof; or (iii) at least one triterpenoid saponin, a derivative thereof, a metabolite thereof, or a biosynthetic intermediate thereof.

6. The genetically modified plant cell according to claim 5 wherein said
(e) biosynthetic intermediate of a steroidal alkaloid is cholesterol; or
(f) biosynthetic intermediate of said triterpenoid saponin is medicagenic acid, oleanolic acid, augustic acid, glucuronic acid, glycyrrhetinic acid, or any combination thereof, or
(g) any combination thereof.

7. The genetically modified plant cell according to claim 1, wherein said plant cell is a cell from a plant in the Poales order, the Caryophyllales order, the Solanales order, the Fabales order, the Malvales order, the Apiales order, the Brassicales order, the Asparagales order, the Dioscoreales order, or the Liliales order.

8. The genetically modified plant cell according to claim 7, wherein:
(a) when said plant cell is a cell from a plant in the Poales order, said plant is of the Oryza genus, the Hordeum genus, the Avena genus, or the Triticum genus;
(b) when said plant cell is a cell from a plant in the Caryophyllales order, said plant is of the Spinacia genus, the Chenopodium genus, the Beta genus, the Rheum genus, the Vaccaria genus, the Saponaria genus, or the Gypsophila genus;
(c) when said plant cell is a cell from a plant in the Solanales order, said plant is of the Solanum genus, the Capsicum genus, the Nicotiana genus, the Hyoscyamus genus, the Datura genus, or the Atropa genus;
(d) when said plant cell is a cell from a plant in the Fabales order, said plant is of the Glycyrrhiza genus, the Medicago genus, the Glycine genus, the Lotus genus, the Cicer genus, the Phaseolus genus, the Pisum genus, the Arachis genus, the Lupinus genus, or the Acacia genus;
(e) when said plant cell is a cell from a plant in the Malvales order, said plant is from the Theobroma genus;
(f) when said plant cell is a cell from a plant in the Apiales order, said plant is of the Daucus genus, the Apium genus, the Petroselinum genus, the Panax genus, the Bupleurum genus, the Hedera genus, or the Centella genus; or
(g) when said plant cell is a cell from a plant in the Brassicales order, said plant is of the Arabidopsis genus, the Brassica genus, the Capparis genus, or the Carica genus.

9. The genetically modified plant cell according to claim 8, wherein:
(a) when said plant cell is a cell from a plant in the Caryophyllales order, said plant is spinach, beetroot, or quinoa;
(b) when said plant cell is a cell from a plant in the Solanales order, said plant is tomato, wild tomato, potato, wild potato, eggplant, pepper, bell pepper, cayenne pepper, chili pepper, pimiento, tabasco pepper, ground cherry, tobacco, or bittersweet; or
(c) when said plant cell is a cell from a plant in the Fabales order, said plant is pea, alfalfa, soy, Lotus japonicus, or licorice.

10. The genetically modified plant cell according to claim 9, wherein the plant cell is from a tomato plant, said tomato cell comprises
(a) a decreased amount of alpha-tomatine or a derivative thereof, a metabolite thereof, or a biosynthetic intermediate thereof;
(b) a decreased amount of dehydrotomatine or a derivative thereof, a metabolite thereof, or a biosynthetic intermediate thereof;
(c) a decreased amount of an esculeoside and or a dehydroesculeoside or a derivative thereof, a metabolite thereof, or a biosynthetic intermediate thereof; or
(d) a decreased amount of lycoperoside; or
(e) a combination thereof.

11. The genetically modified plant cell according to claim 10, wherein said esculeoside comprises esculeoside A.

12. The genetically modified plant cell according to claim 9, wherein the plant cell is from a potato plant, said potato plant cell comprises
(a) a decreased amount of alpha-chaconine or a derivative thereof, a metabolite thereof, or a biosynthetic intermediate thereof;
(b) a decreased amount of alpha-solanine or a derivative thereof, a metabolite thereof, or a biosynthetic intermediate thereof; or
(c) a combination thereof.

13. The genetically modified plant cell according to claim 9, wherein the plant cell is from an eggplant plant, said eggplant plant cell comprises
(a) a decreased amount of alpha-solasonine or a derivative thereof, a metabolite thereof, or a biosynthetic intermediate thereof;
(b) a decreased amount of alpha-solamargine or a derivative thereof, a metabolite thereof, or a biosynthetic intermediate thereof; or
(c) a combination thereof.

14. The genetically modified plant cell according to claim 9, wherein the plant cell is from a quinoa plant, said quinoa plant cell comprises a decreased amount of a triterpenoid saponin or a derivative thereof, a metabolite thereof, or a biosynthetic intermediate thereof.

15. The genetically modified plant cell according to claim 9, wherein the plant cell is from a pea plant, said pea plant cell comprises a decreased amount of soyasaponin I or soyasaponin VI or a combination thereof.

16. The genetically modified plant cell according to claim 1, wherein said plant cell comprises a leaf cell, a petiole cell, a plant stem or stalk cell, a root cell, a bud cell, a tuber cell, a bean cell, a grain or kernel cell, a fruit cell, a nut cell, a legume cell, a seed or seed cell, a callus cell, a bract cell, or a flower cell.

17. The genetically modified plant cell of claim 1, wherein said CSLG gene comprises a mutation and said mutation comprising at least one or more point mutations, or an insertion, or a deletion, or any combination thereof, and wherein said CSLG enzyme expressed from said gene has decreased stability or decreased activity or both.

18. The genetically modified plant cell of claim 1, wherein said CSLG gene is selectively silenced or repressed.

19. The genetically modified plant of claim 17, wherein said cell further comprises at least one silencing molecule targeted to a polynucleotide of said CSLG gene, wherein the silencing molecule is an RNA interference molecule or an antisense molecule, or wherein the silencing molecule is a component of a viral induced gene silencing system.

20. The genetically modified plant cell according to claim 18, wherein:
(a) when said CSLG gene has the nucleic acid sequence of SEQ ID NO: 30, the silencing molecule comprises the sequence of SEQ ID NO: 42 or a complementary sequence thereof, or a homolog thereof having at least 90% identity to the nucleic acid sequence set forth in SEQ ID NO: 42 or a complementary sequence thereof;
(b) when said CSLG gene has the nucleic acid sequence of SEQ ID NO: 34, the silencing molecule comprises the sequence of SEQ ID NO: 43 or a complementary sequence thereof, or a homolog thereof having at least 90% identity to the nucleic acid sequence set forth in SEQ ID NO: 43 or a complementary sequence thereof;
(c) when said CSLG gene has the nucleic acid sequence of SEQ ID NO: 38, the silencing molecule comprises the sequence of SEQ ID NO: 44 or a complementary sequence thereof, or a homolog thereof having at least 90% identity to the nucleic acid sequence set forth in SEQ ID NO: 44 or a complementary sequence thereof,
(d) when said CSLG gene has the nucleic acid sequence of SEQ ID NO: 65 or SEQ ID NO: 93, the silencing molecule comprises the sequence of SEQ ID NO: 106 or a complementary sequence thereof, or a homolog thereof having at least 90% identity to the nucleic acid sequence set forth in SEQ ID NO: 106 or a complementary sequence thereof; or
(e) when said CSLG gene has the nucleic acid sequence of SEQ ID NO: 95, the silencing molecule comprises the sequence of SEQ ID NO: 107 or a complementary sequence thereof, or a homolog thereof having at least 90% identity to the nucleic acid sequence set forth in SEQ ID NO: 107 or a complementary sequence thereof.


SEQUENCE LISTING (130 sequences)

SEQ ID NO: 1 - DNA, 35 bp, synthetic construct
  misc_feature 1..35: note = Syn. Polynucl.
  source 1..35: mol_type = other DNA
aaaaagaattccggatcttctctcgaactggtcaa

SEQ ID NO: 2 - DNA, 39 bp, synthetic construct
  misc_feature 1..39: note = Syn. Polynucl.
  source 1..39: mol_type = other DNA
aaaaagaattccactttcattgcttcatccattagatct

SEQ ID NO: 3 - DNA, 37 bp, synthetic construct
  misc_feature 1..37: note = Syn. Polynucl.
  source 1..37: mol_type = other DNA
aaaaagaattccttagcttatggccacatcacacctt

SEQ ID NO: 4 - DNA, 37 bp, synthetic construct
  misc_feature 1..37: note = Syn. Polynucl.
  source 1..37: mol_type = other DNA
aaaaagaattcactcaagatttggtgaagctgtggtt

SEQ ID NO: 5 - DNA, 36 bp, synthetic construct
  misc_feature 1..36: note = Syn. Polynucl.
  source 1..36: mol_type = other DNA
aaaaaggcgcgccaatcatagagaagaaagaagacg

SEQ ID NO: 6 - DNA, 38 bp, synthetic construct
  misc_feature 1..38: note = Syn. Polynucl.
  source 1..38: mol_type = other DNA
aaaaagcggccgcactcctgcaggaattgtcatttctc

SEQ ID NO: 7 - DNA, 43 bp, synthetic construct
  misc_feature 1..43: note = Syn. Polynucl.
  source 1..43: mol_type = other DNA
aaaaagcggccgcatgagtattgtaattgatgatgatgaaatc

SEQ ID NO: 8 - DNA, 33 bp, synthetic construct
  misc_feature 1..33: note = Syn. Polynucl.
  source 1..33: mol_type = other DNA
aaaaggcgcgcccacacgccacagatggttctt

SEQ ID NO: 9 - DNA, 59 bp, synthetic construct
  misc_feature 1..59: note = Syn. Polynucl.
  source 1..59: mol_type = other DNA
ggggacaagtttgtacaaaaaagcaggctatgagtattgtaattgatgatgatgaaatc

SEQ ID NO: 10 - DNA, 54 bp, synthetic construct
  misc_feature 1..54: note = Syn. Polynucl.
  source 1..54: mol_type = other DNA
ggggaccactttgtacaagaaagctgggttcatactaccttctgtcctaagcct

SEQ ID NO: 11 - DNA, 55 bp, synthetic construct
  misc_feature 1..55: note = Syn. Polynucl.
  source 1..55: mol_type = other DNA
ggggacaagtttgtacaaaaaagcaggctatgaatattgcaattgatgatgatga

SEQ ID NO: 12 - DNA, 59 bp, synthetic construct
  misc_feature 1..59: note = Syn. Polynucl.
  source 1..59: mol_type = other DNA
ggggaccactttgtacaagaaagctgggttcatttgtatcaacatttgtaaattcacac

SEQ ID NO: 13 - protein, 219 aa, Solanum lycopersicum
MSIVIDDDEIFSLPSLDELESITHLLYDDDSDFFETLSPMSLDVTTLLPNIPTSNSIESPVTPEETKEPSVACEDAPQDWRRFIGVRRRQWGTFSAEIRDPNRRGARLWLGTYESPRDAALAYDQAAYKIRGTKVRLNFPDLIGSDVPMPPRVTARRRTRSRSRSPEPLTTSSSSSSSSSSSSSSSSENGTKKRKIDLINSIAKSKLLCGMDLQMLIQM

SEQ ID NO: 14 - protein, 222 aa, Solanum tuberosum
MNIAIDDDEIFSLPSLDELESITHLLYDDDSDFFETLSPMSLDSTTLLPNNPTPNSLESPVRPEGTKETFVAREHEESAPQDWRRFIGVRRRQWGTFSAEIRDPNRRGARLWLGTYESPQDAALAYDQAAYKIRGTKARLNFPDLIGSDVPMPPRVTARRRTRSRSRSPEPSTTSSSSSSSSSSSSSSSMENGTKKRKIDLINSIAKAKLLCGVNLQMLIQM

SEQ ID NO: 15 - DNA, 988 bp, Solanum lycopersicum
  source 1..988: mol_type = unassigned DNA
taatattacattacattcatcacatattattcatccaaaacaagaatgagtattgtaattgatgatgatgaaatcttctctttacctagccttgatgaacttgaatccatcacacatcttctttatgacgacgattccgattttttcgaaactctttccccaatgagtttagatgttacaacattattgcctaatattcctacctccaattcaattgaatcccccgtaacaccggaggaaacaaaagaaccatctgtggcgtgtgaggacgcgccacaagattggaggcggttcataggggtgaggcggaggcagtggggcacgttttcagccgaaataagagatccaaataggagaggagcgaggctgtggctcggaacttatgagtccccgagggatgcagcattagcttatgaccaagccgcttacaagattcggggaaccaaagttcggcttaattttcctgacctgattggctcggacgtacctatgccacctagagtaacggctaggcgtcgtacacgctcacgctcacgctcacccgagccattaacaacttcgtcctcgtcatcctcatcatcctcgtcctcgtcctcgtcctcgtcggaaaatggaacgaagaaaaggaaaatagatttgataaactcaatagcaaaatccaaattactttgtgggatggatttacaaatgttaatacaaatgtgagaaaagagcaaaggtttatttttcttcgtttgacaattaagtactacgtcgtataattaatagactcatcaaggtcattgtgtaaatgcacttctttcacgaccttctcctttatgagattgttatgaattttacattatttcctttatcaactatatatttatcgttttcatacgcggtggagttcatctgaatttctctttctaaggttatatatagagaaggatgttgaatttttcgtcttcttttttttattaaataaaaaatctatcttctacatcag

SEQ ID NO: 16 - DNA, 754 bp, Solanum tuberosum
  source 1..754: mol_type = unassigned DNA
gaaaatttcattcatccaaaagaagaatgaatattgcaattgatgatgatgaaatcttctctttacctagcctcgatgaacttgaatctatcacacatcttctttatgatgatgattccgatttttttgaaactctttcaccaatgagtttagatagcacaacattattgcctaataatcctactccaaattcacttgaatcccccgtaagaccggagggaacaaaggaaacatttgtggcgcgcgaacacgaagaaagcgcgccacaagattggaggcggttcataggagtgaggcgaaggcagtggggcacgttttcagccgaaataagagatccaaataggagaggcgcgaggctgtggctaggaacttatgagtccccgcaggatgcagcattggcttatgaccaagctgcttacaagattcggggtaccaaagctcggctcaattttccggacttaattggctcggacgtgcctatgccaccaagagtaacggctaggcgtcgtactcgctcacgctcgcgctcacccgagccatcaacaacttcttcgtcctcatcctcgtcctcgtcctcatcctcgtcctcgtccatggaaaatgggacgaaaaaaaggaaaatagatttgataaactcaatagccaaagccaaattactctgtggtgtgaatttacaaatgttgatacaaatgtgagaaaagagcaaaggtttatttttttcttcgtttaacaattaagtattacgtataattaa

SEQ ID NO: 17 - protein, 224 aa, Solanum lycopersicum
MPSLGVFSILISRMACYIIVNLSSLIAISRSTPGPVEGSVEHSQIIRNGSTHEDDIVINPTLLASVQSFVEPNLTAAALYRATHDSHMAADEAIAFNMPLQPNLFENASVEPSPDAEHPSQTQSLCWPDKRDTIESEVLSYGRNDQEEVKFDGEAVGRSHAYSQRLLNIINQTLASVGVDPSLADVRVQLDISKKTSSGATTTRLSSGENYGGAPKRLRTEGSM

SEQ ID NO: 18 - DNA, 2497 bp, Solanum lycopersicum
  source 1..2497: mol_type = unassigned DNA
atgccaagtttaggggtgttttcaatactcatctctagaatggcttgctatatcattgttaatttaagttctcttattgctatttctgctgttgagtaaaaagcatagtagttccttgcatcatttgcactttcatctgtttgatgttatgctgtggattcttttcctaagtgttgactttttctctacctccctatttaggaaaaagaggcaatagtattttaagtccatctcttaaggaatggctaatgttgaaatataacaaagaacttccttctttggcaacacatggttggcttcttgtcctttagctatgtgattttctgtacttcgattttattccctcctccacttcttatttggcagtttagttgtagcattaaaatagattcattctaaccagatggcttacttatggaatcttcaactctttaataatagtaatggaaaattatgaagtcagtgcaccatgagaaagaagacattgtttgacccaagagaagtaaactgagctatataaaattggattgagcgctttaattactgcagatattaccctctgaaaagtactggatcaaagaaaaaaatgttctctgatgttacctatacctgtatgccccagttgctgtacagtaaaggtataattcagtagtcatttcgtatgcttgccaaatacagaaaaatgcagacgttagctgtatttctaggggaaactcctcgcctacagttcaaacaaaggttttacatttgcttataatttcctccctccaaagcaaagtgaccggattttgggctcttttaggaggagagttgggcacaactttaggatggaagcagtaatgcttttctggaagtaaaactaatgctcttctcttattattgacagagaagtactcctgggcccgtggaaggctctgttgaacattctcaaataatcagaaacggctctactcatgaggatgatattgtcattaacccaacattgcttgcaagtgtccagagctttgtagaaccgaacttgactgctgctgctttatatagagcaacacacgattctcatatggcagcggatgaggcaattgcctttaacatgccactgcaacctaatttatttgaaaatgcatctgttgaaccatctcctgatgctgagcacccttctcagacacaatcattatgttggccagataaacgagatacaattgagtcggaggttctgagctatggcagaaatgatcaagaagaagtgaaattcgatggtgaagcagttggaagatcacatgcatatagtcaaaggtaagatgatttatcaggagttcaatagctatgacttgatgtccttgtaaggtggaaattcaaatttatttcttctatgaccccatgacttgctaatttctgtaatgatgccaaacttgtattacacctacgaagtaggcatgtgatacagtatcactttaagtcccttggacccagtgggcctagtggcagtcacggtcttagaagaattatcctatggttgtcaagtgcatgaaatagatttagactagttaatgtttctcgtggttattagctggttggctagaatgcaaagtgtagcctttttaagccccttccagcatgagtttttttgtaaaacctgctgtaacttgtgggtttgcattttttttgtgaataaaattgccagttcaacaaagatttcagtggcttgaaggaagtcattttatatgacccggcatggtttacctgttgaaggttaataacaagcggaaccctggatttcgagatttgagtctcactttaggatttttcagacttcccattaacacaaagtcatgtataacacacatgttcgtatcattcttacttgtgcagttgtcctctgtacctttaggcacattttaatctgaactcggttgatctgaaattatattatgatgccagtaaactactgattttggattctatttatgtgacatattgggtcttggtattgagcaggttgcttaatatcataaaccagactctagcatctgtgggagtggatccgtcactggccgatgttagagtacagcttgatatcagcaaaaaaacaagcagtggagccacaactacaagattaagcagtggagagaactatggtggtgctcctaaaaggcttaggacagaaggtagtatgtgattattaatctagcatggctccactcctaatttttctgcatcttgtcatcgttttgatggggagatagttgaagtggttggtctccgtggatgaggtggtgcacaaacagcttatggttgtccagttaggtttccatttaaatatgagaagctgcattgtcattcttaagggtatttagttttgaattgagataagtcgactttgatagttctgtcagtgtgatatggttatgcctatcgatttgccatggatctgttttcgtagttgatatttaaacagggaaatttgaagttgtttcaaatgttagcatgaagaatttta

SEQ ID NO: 19 - DNA, 996 bp, Solanum lycopersicum
  source 1..996: mol_type = unassigned DNA
atgccaagtttaggggtgttttcaatactcatctctagaatggcttgctatatcattgttaatttaagttctcttattgctatttctagaagtactcctgggcccgtggaaggctctgttgaacattctcaaataatcagaaacggctctactcatgaggatgatattgtcattaacccaacattgcttgcaagtgtccagagctttgtagaaccgaacttgactgctgctgctttatatagagcaacacacgattctcatatggcagcggatgaggcaattgcctttaacatgccactgcaacctaatttatttgaaaatgcatctgttgaaccatctcctgatgctgagcacccttctcagacacaatcattatgttggccagataaacgagatacaattgagtcggaggttctgagctatggcagaaatgatcaagaagaagtgaaattcgatggtgaagcagttggaagatcacatgcatatagtcaaaggttgcttaatatcataaaccagactctagcatctgtgggagtggatccgtcactggccgatgttagagtacagcttgatatcagcaaaaaaacaagcagtggagccacaactacaagattaagcagtggagagaactatggtggtgctcctaaaaggcttaggacagaaggtagtatgtgattattaatctagcatggctccactcctaatttttctgcatcttgtcatcgttttgatggggagatagttgaagtggttggtctccgtggatgaggtggtgcacaaacagcttatggttgtccagttaggtttccatttaaatatgagaagctgcattgtcattcttaagggtatttagttttgaattgagataagtcgactttgatagttctgtcagtgtgatatggttatgcctatcgatttgccatggatctgttttcgtagttgatatttaaacagggaaatttgaagttgtttcaaatgttagcatgaagaatttta

SEQ ID NO: 20 - DNA, 3899 bp, Solanum tuberosum
  source 1..3899: mol_type = unassigned DNA
tttttttacaaaaaactttttatccaacaccaccgagtagcttgactcgcccccttaaaaaattattttaaaaataaaatattttttttttcttatcccactcctctcccctaaaaaaaaaataagttcaaaagaattctttttgggggtgagtgggtagtgaggtgggaggctagggggggtactgggtagaggatggggtgggtgataagaaaaaataaatcttcaaaaagaaaaaaaagttcttttatcttcttaaattgctaaattcttaacatttaattatttaaatgttctttaaaaaaatttctattacatatatttatgtgtacacaccattaccattttcaaaaaaaataaatatttatgtatacacaacgtcaacagaaaattctacatatatgcccatgtggcataagaagggtgtttttaaattcacttaatcaagtaaaggggtgtttttaaggctgttaatagttggaggattaaagtaataattcatgccaagtttaggggtgttttcaatacttatctctagaatggtctccctattccttgctatattgttgttaatttaagttctcttattgctattctgctatgttgagtaaaaagcacagtagttccttgcatcatttgcacttctcatctgtttgatggtatgctgtggattcttttttcaagtggtgttggacttgtttgcttggatgataatctttcatgtttactccttattgttgaactttttttctacctccctattaaggaaaaaaaggcaatagtattttcagtccatctcttaaggaatggctaatgttgaagatataatgaagaacttccttctttggcaacacatggttggcttcttgtcctctagctatgtgatattttatacttcgatttttattccctccttcacttcttgtttggcagtttagttatagcattaaaatagattcattataaccagatggcttactgaaggaatcttctactctttaataatagtgttaaattaggtcttaggcctaactcacaccccaaaagctagctcaaagggaggaggattgttcaagccttgtaaggagtccacccatctcaaagggaggaggcttgttcaagccttataaggagtccacccatctcattaaccaccgatgtgggacttttgtcattctttaacaaatagtattagaaaattatgaagtcagtgcaccatgagaaagaagacattgtttgacccaagagaagtaaactgagctatataaaatcggattgagactttaatttactgcagatattaccctctgaaaagtactggattaaagaaaaaaatgttctctgatgttaccctatacctgtgtgccccagttactgtacagtaaagtcataattcagtagttattttggatgctttccaaatacagaaaaatgcagacgttagctgtttttgtaggggaaactcctcgcctatggttcaaacaaaggctttacatttgtttttaattttctccctccaaagcaaagttaccggatttcaggctgttttaggaggagagttgggcacaactttaggatggaagcagtagtgtttttctgaaagtaaaactaatgctcttctcttattattgacagagaagcactcctgggcccgtggaaggctctgttgaacattctcaaataatcagaaacggctctactcatgaggatgatattgtcattaacgcaacattgctttcgagtgcccagagctttgtagaaccgaacttgactgctgctgctttatatagagcaacacacgattctcatatggcagcggatgaagcaattgcctttaacatgccactgcaacctaatttatttgaaaatgcatctgttgaaccatctcctgatgctgagcacccttcccagccacaatcattatgttggccaggtaaacgagatacaattgagtcggaggttctgagctatggcagaaatgatcaggaagaagtgaaatgcgatggtgaagcagttgcaagatcacatgcgtatactcaaaggtaagatgatttatcacgagttcaatagctatgacttgatgtcctggtaaggtggaaattcaaatttatttcttctacgcccccatgacttgctaatttctgtaatgatgccaaacttgtattacacctacgaaataggcatgtgatacagaatcactttaagtaccttggacccagtgggcctagtggcagtcaaggtcttagaagaattacctgtagtgttccaatttcttattgtcctgtgattgccaagtgcatgaaatagattcagactaggtaatgtttctcgtggttattagctggttggttgaaatgcagattgtagcctttctgagccccttccagtatagttttttttgtaaaacctgctgcaacttgtgggtttgcatttttttgtgaataaaattgccaattcaaaaaagatttcagtggcttgaaggaagtcatttatatgacccggcattgtttacccgagcaatcaaatatcaatcaggtttccctgcatggcttcccccaacttttctacctgacccatcaaatggaaatctaagttgaaggttaataacaagcggatctctggatttcgaggtttgagtctcacttaaggattttccagacttcccattaaaacgaagaaatgtataacaaacatgtacatatcattctgacttgagcagttgtcctctgaacctttaggcacattctgatctgatttcagttgatctgaaattatattatgatgctagtatactactgattttggattctatttatgtgacatattgagtcttggtattgagcaggttgcttaatatcataaaccagacactagcatctgtgggagtggatccttcactggccgatgttagagtacagcttgatatcagcaaaaaaccaagcagtggagccacaactacaacattaagcagtgaagagaactatgatggtgctcctaaaaggcttaggacagaaggtagtatgtgattgtcaatctagcatggttccactcctaatttttctgcatcttgtcattgtttcgatggggagatacttgaagtggttggtctctgtggatgaggtggtgcacaaacagcttatggttgtccagttaggtttccatttaaatatgagaagctgcattgtcattcttaagggtatttagatagtcgactttggaaattctgtcagtgtgatgtggttatgcctatcgatttgagatgcctccatggatctggtttcatagttgatatttaaacagggaaatttgaagttgtttcaaatgtcagcatgaagaattttatgtacattaccaaatcttttccttttcagtattttgtgattagttcacttaaacaggatgctggcttttcaattgtgttttcagaaataaaagtcagcacttgtatcattgtgaaaaactgaaaattttggtctttaagtcgaatcaacaacataatgcaagtatttactgataacggcgtttggtcagatgaatacggcagtttcacaatgattgcatatgaatatgctcatgttagtccatggtatatattgtaattttatcctaaagatatcgtaatgagaagttagatgagtttgatgcgatgaactgatgaagcattggtaatgggttattggtttagcagttttgctaattctcatttatatttgggatatccgttgtcaaatgtttgaggttcttttcttaacacattaatcgaattaataaattaaactctcggctattctactaggtgccaatatttgcttttgagcaagatgcaatatgtcgttcatttggtttgtcaccttgtttctaagtgagttttaatctataacagaatgtttgttggtaaa

SEQ ID NO: 21 - DNA, 990 bp, Solanum tuberosum
  source 1..990: mol_type = unassigned DNA
atgccaagtttaggggtgttttcaatacttatctctagaatggcttgctatattgttgttaatttaagttctcttattgctattagaagcactcctgggcccgtggaaggctctgttgaacattctcaaataatcagaaacggctctactcatgaggatgatattgtcattaacgcaacattgctttcgagtgcccagagctttgtagaaccgaacttgactgctgctgctttatatagagcaacacacgattctcatatggcagcggatgaagcaattgcctttaacatgccactgcaacctaatttatttgaaaatgcatctgttgaaccatctcctgatgctgagcacccttcccagccacaatcattatgttggccaggtaaacgagatacaattgagtcggaggttctgagctatggcagaaatgatcaggaagaagtgaaatgcgatggtgaagcagttgcaagatcacatgcgtatactcaaaggtaggttgcttaatatcataaaccagacactagcatctgtgggagtggatccttcactggccgatgttagagtacagcttgatatcagcaaaaaaccaagcagtggagccacaactacaacattaagcagtgaagagaactatgatggtgctcctaaaaggcttaggacagaaggtagtatgtgattgtcaatctagcatggttccactcctaatttttctgcatcttgtcattgtttcgatggggagatacttgaagtggttggtctctgtggatgaggtggtgcacaaacagcttatggttgtccagttaggtttccatttaaatatgagaagctgcattgtcattcttaagggtatttagagtcgactttggaaattctgtcagtgtgatgtggttatgcctatcgatttgagatgcctccatggatctggtttcatagttgatatttaaacagggaaatttgaagttgtttcaaatgtcagcatgaagaatttta

SEQ ID NO: 22 - protein, 342 aa, Solanum lycopersicum
MADLLSNWSSTLEAVPKSHCIPEHERPSDPVEIGDSIPVIDLGKANGEERSVVVKDLLKAFEEYGFFQIINHGVPVDLMDEAMKVYKEFFSLPAEEKENYAKDAANNTNRGAATLYSSSAKHYDSEEHRYWRDVLEHSCNLDGEDKKTWPDNPPRYREVIGAYGDELRRVSKVILGMLSEGLGLEAGFFDKELGQRMLVNHYPACPNPSLTLGVGGHCDPNLITIIQQEVYGLQILKDDKWIGVQPIRNAFVVNSGLPITVYSNGKLTSVAHRVVTNTTESRTSIGTFICPHEIVEPAKALVGPENPPQFKPFHWGIDFMPHYLSKKSVYHASLEPFKTEAN

SEQ ID NO: 23 - protein, 341 aa, Solanum tuberosum
MADLLSNWSSTLEAVPPSHCIPVHERPSDPVEIVDNIPVIDLGKANGEERSVVVKELLKAFEEYGFFQIINHGVPVDLMDEAMKVYKEFFSLPAAEKAEYAKDAANDTNRGAATLYSSSAKHYDSEEHRYWRDVLEHSCNLDGKDKKTWPSNPPRYREVIGAYGDELRRVSKVILGLLAEGLGLEAGFFDTELGQRMLVNHYPACPDPSLTLGVGGHCDPNLITIIQQEVYGLQILKDDKWIGVQPIRNAFVVNSGLPITVVSNGKLTSVAHRVVTNTTHSRTSIGTFICPHDIVEPAKALVGPENPPQFKSFNWGIDFMPHYLSKKSVYHASLEPFKIDA

SEQ ID NO: 24 - DNA, 1726 bp, Solanum lycopersicum
  source 1..1726: mol_type = unassigned DNA
aaaaaatatttgtttttaaaatgtgtaatttttagtggcatgctctaaaaaaaaataaaattatctgagcatcagttttggttatgcaatttccttccctataaatggccctccatatctcaaatgagatatcaaacaatttgcagaagtagtagtattaacatttagaagataactttgtttcccaggtaaataaatcaataaatcctccttttctttgtttgtttgtttatttgttgagatatttatgatttttggttttggtttagattgattgtcaatggcggatcttctctcgaactggtcaagcacattagaagcagttcctaaaagtcattgcatcccagagcatgaaagaccatcagatccagttgaaattggcgacagtattccagtcattgatttgggaaaagctaatggtgaagaacgaagtgttgttgttaaagatctgttgaaagcttttgaagaatatgggttttttcaggtacgcaactctgtttcttttttttttgttcccgttaatgtgaaattgaaatgatgatatatgaacaaacagataatcaatcatggagtacctgtagatctaatggatgaagcaatgaaagtgtacaaagaatttttcagtcttccagctgaagaaaaagaaaattatgcaaaagatgcagctaataataccaataggggtgcagctacactgtacagtagcagtgctaagcattatgattcagaggagcatcgttactggagagatgtgttggaacatagctgcaatcttgatggagaagacaaaaaaacttggcccgataaccctccaagatataggtacctacctatctaaactatgtatggtttagcaattaatttccctcttttcttacacatgtattttggttgtacttcagggaggttattggtgcctatggtgatgaattgagaagggtgagcaaagttatcttgggtatgttaagtgaagggctaggtttggaggcagggttctttgacaaagaacttgggcagagaatgcttgtgaatcactatccagcatgtccaaatccaagtttaactttgggagttggtggacattgtgatcctaatctcataaccattatccaacaagaagtctatggtcttcaaatattgaaggatgacaaatggattggtgtgcagcctattcgcaatgcatttgtggttaattctggtttaccaattacggtatgtatgtgtgtaggtcttctctaacaccccctttttttcttctcttataatgtttgctatgcatacaggtatatagcaatggaaagctaactagtgttgcacatcgtgtggtgacaaacacaactgagtcacgaacctccattggtacttttatttgcccacatgagattgttgaacctgcaaaagcacttgttggtcctgagaatcctccacagttcaaacccttccattggggaatcgattttatgccacattacctcagcaagaaatcagtgtaccacgcttcattggagcccttcaaaacagaagctaattaagcattaaggatatatcaaatctatgctgctgctgctactacttcttttaatttccactgaaataagagctttaattcaaaatgtctttctagtttgtattctacttacttcatgaataagaaacttccaatcctattctctactggtttcgatctacatgaatattttattatttccattgcattttcaatcag

SEQ ID NO: 25 - DNA, 1422 bp, Solanum lycopersicum
  source 1..1422: mol_type = unassigned DNA
aaaaaatatttgtttttaaaatgtgtaatttttagtggcatgctctaaaaaaaaataaaattatctgagcatcagttttggttatgcaatttccttccctataaatggccctccatatctcaaatgagatatcaaacaatttgcagaagtagtagtattaacatttagaagataactttgtttcccagattgattgtcaatggcggatcttctctcgaactggtcaagcacattagaagcagttcctaaaagtcattgcatcccagagcatgaaagaccatcagatccagttgaaattggcgacagtattccagtcattgatttgggaaaagctaatggtgaagaacgaagtgttgttgttaaagatctgttgaaagcttttgaagaatatgggttttttcagataatcaatcatggagtacctgtagatctaatggatgaagcaatgaaagtgtacaaagaatttttcagtcttccagctgaagaaaaagaaaattatgcaaaagatgcagctaataataccaataggggtgcagctacactgtacagtagcagtgctaagcattatgattcagaggagcatcgttactggagagatgtgttggaacatagctgcaatcttgatggagaagacaaaaaaacttggcccgataaccctccaagatatagggaggttattggtgcctatggtgatgaattgagaagggtgagcaaagttatcttgggtatgttaagtgaagggctaggtttggaggcagggttctttgacaaagaacttgggcagagaatgcttgtgaatcactatccagcatgtccaaatccaagtttaactttgggagttggtggacattgtgatcctaatctcataaccattatccaacaagaagtctatggtcttcaaatattgaaggatgacaaatggattggtgtgcagcctattcgcaatgcatttgtggttaattctggtttaccaattacggtatatagcaatggaaagctaactagtgttgcacatcgtgtggtgacaaacacaactgagtcacgaacctccattggtacttttatttgcccacatgagattgttgaacctgcaaaagcacttgttggtcctgagaatcctccacagttcaaacccttccattggggaatcgattttatgccacattacctcagcaagaaatcagtgtaccacgcttcattggagcccttcaaaacagaagctaattaagcattaaggatatatcaaatctatgctgctgctgctactacttcttttaatttccactgaaataagagctttaattcaaaatgtctttctagtttgtattctacttacttcatgaataagaaacttccaatcctattctctactggtttcgatctacatgaatattttattatttccattgcattttcaatcag

SEQ ID NO: 26 - DNA, 1619 bp, Solanum tuberosum
  source 1..1619: mol_type = unassigned DNA
ctttgttatgcaattttcttccctataaatggccctccatagctcaaatgagatatcagacaatttaaagaagtactattaacatttagaagatttctttctttcccaggtaaataaatcattttccctctttccttcttgctctttctttgtttatttgttcagatttttaccctttttgttttggttagattcattgacaatggcggaccttctttcaaactggtcaagcacattagaagcagttcctccaagtcattgcatcccagtgcatgaaagaccatcggatccagttgaaattgtggacaatattccagtcattgatttgggaaaagctaatggtgaagaacgaagtgttgttgttaaagaacttttgaaagcttttgaagaatatgggttttttcaggtttattatttatacaatagtacaactctgttcttttttctttttttttcttattgtatttaaaaatgatctgaaattgaaatgatgaaatagataatcaatcatggagtacccgtagatctaatggatgaagcaatgaaagtgtacaaagaatttttcagtctgccagcagcagagaaagcagaatatgcaaaggatgcagctaatgatacaaataggggtgcagctacactgtacagtagcagcgctaagcattatgattcagaggagcatcgttactggagagatgtcttggaacatagctgcaatcttgatgggaaagacaaaaaaacttggcctagtaaccctccaagatataggtacctacctaaactatgcttagcaaaattccctcttgttatttttcttacctagtatttgcttgtccttcagggaggttattggtgcatatggagatgaattgagaagggtgagcaaagttatcttgggtctgttagctgaagggctaggtttggaggcagggttctttgacacagaacttgggcagagaatgcttgtgaatcactatccagcatgcccagatccaagtttaaccttgggagttggtggacattgtgatcctaatctcataaccattatccaacaagaagtgtatggtcttcaaatattgaaggatgacaaatggattggtgtgcagcctatccgcaatgcatttgtggtcaattctggtttaccaattacggtaggtgtaacactttctcttaattttcatggtctacaagcgattctcttattgctctgttttttttgtataaatacaggtagttagcaatggaaagctaactagtgttgcacatcgtgtggtgacaaacacaactcattcacgaacctccattggtacttttatttgcccacacgatattgttgagcctgcaaaagcacttgttggtccggagaatcctccacagttcaaatcctttaattggggaattgattttatgccacattacctcagcaagaaatcagtttaccacgcatcattggagcccttcaaaatcgatgcttaagcatttgtgtgccagaaggatcaagtctatgctgctacttttaatttccactaaaataagagctttaatttacaatgtctttctagtttgtatcctacctttgttacctatttcatgaataagaatctttctttcctattctcttc

SEQ ID NO: 27 - DNA, 1292 bp, Solanum tuberosum
  source 1..1292: mol_type = unassigned DNA
ctttgttatgcaattttcttccctataaatggccctccatagctcaaatgagatatcagacaatttaaagaagtactattaacatttagaagatttctttctttcccagattcattgacaatggcggaccttctttcaaactggtcaagcacattagaagcagttcctccaagtcattgcatcccagtgcatgaaagaccatcggatccagttgaaattgtggacaatattccagtcattgatttgggaaaagctaatggtgaagaacgaagtgttgttgttaaagaacttttgaaagcttttgaagaatatgggttttttcagataatcaatcatggagtacccgtagatctaatggatgaagcaatgaaagtgtacaaagaatttttcagtctgccagcagcagagaaagcagaatatgcaaaggatgcagctaatgatacaaataggggtgcagctacactgtacagtagcagcgctaagcattatgattcagaggagcatcgttactggagagatgtcttggaacatagctgcaatcttgatgggaaagacaaaaaaacttggcctagtaaccctccaagatatagggaggttattggtgcatatggagatgaattgagaagggtgagcaaagttatcttgggtctgttagctgaagggctaggtttggaggcagggttctttgacacagaacttgggcagagaatgcttgtgaatcactatccagcatgcccagatccaagtttaaccttgggagttggtggacattgtgatcctaatctcataaccattatccaacaagaagtgtatggtcttcaaatattgaaggatgacaaatggattggtgtgcagcctatccgcaatgcatttgtggtcaattctggtttaccaattacggtagttagcaatggaaagctaactagtgttgcacatcgtgtggtgacaaacacaactcattcacgaacctccattggtacttttatttgcccacacgatattgttgagcctgcaaaagcacttgttggtccggagaatcctccacagttcaaatcctttaattggggaattgattttatgccacattacctcagcaagaaatcagtttaccacgcatcattggagcccttcaaaatcgatgcttaagcatttgtgtgccagaaggatcaagtctatgctgctacttttaatttccactaaaataagagctttaatttacaatgtctttctagtttgtatcctacctttgttacctatttcatgaataagaatctttctttcctattctcttc

SEQ ID NO: 28 - DNA, 220 bp, synthetic construct
  misc_feature 1..220: note = Syn. Polynucl.
  source 1..220: mol_type = other DNA
atgagtattgtaattgatgatgatgaaatcttctctttacctagccttgatgaacttgaatccatcacacatcttctttatgacgacgattccgattttttcgaaactctttccccaatgagtttagatgttacaacattattgcctaatattcctacctccaattcaattgaatcccccgtaacaccggaggaaacaaaagaaccatctgtggcgtgtg

SEQ ID NO: 29 - DNA, 251 bp, synthetic construct
  misc_feature 1..251: note = Syn. Polynucl.
  source 1..251: mol_type = other DNA
cggatcttctctcgaactggtcaagcacattagaagcagttcctaaaagtcattgcatcccagagcatgaaagaccatcagatccagttgaaattggcgacagtattccagtcattgatttgggaaaagctaatggtgaagaacgaagtgttgttgttaaagatctgttgaaagcttttgaagaatatgggttttttcagataatcaatcatggagtacctgtagatctaatggatgaagcaatgaaagtg

SEQ ID NO: 30 - DNA, 2142 bp, Solanum lycopersicum
  source 1..2142: mol_type = unassigned DNA
atgaaaaaaaccatggagctcaacaaaagcactgttccacaacctatcaccaccgtataccgactccacatgttcatccactcaataatcatgcttgcattaatatactaccgtgtatctaatttgtttaaattcgaaaacattctcagtttacaagcacttgcttgggcgctcatcacttttggtgaatttagtttcattctcaagtggttcttcggacaaggtactcgttggcgccccgttgaacgagatgttttccctgaaaacattacttgcaaagattccgatctaccgccaattgacgtaatggtattcactgccaatcctaagaaagagccaattgtagatgtcatgaacactgtgatatccgcaatggctcttgattatcccaccgataaattggctgtgtatctcgctgatgatggaggatgtccattgtcgttgtacgccatggaacaagcgtgtttgtttgcaaagctatggttacctttctgtagaaactatggaattaaaacgagatgcccaaaagcatttttttctccgttaggagatgatgaccgtgttcttaagaatgatgattttgctgctgaaatgaaagaaattaaattgaaatatgaagagttccagcagaaggtggaacatgctggtgaatctggaaaaatcaatggtaacgtagtgcctgatagagcttcgcttattaaggtaataaacgagagggagaacgaaaagagtgtggatgatatgacgaaaatgcccttgctagtttatgtatcccgtgaaagaagattcaaccgtcttcatcatttcaagggtggatctgcaaatgctctacttcgagtttctggaataatgagtaatgccccctatgtactggtgttagattgtgatttcttctgtcatgatccaatatcagctaggaaggcaatgtgttttcatcttgatccaaagctatcatctgatttagcctatgttcagttccctcaagtcttttacaatgtcagcaagtcagatatttatgatgtcaaaattagacaggcttacaagacaatatggcatggaatggatggtatccaaggcccagtgttatctgggactggttattttctcaagaggaaagcgttatacacaagtccaggagtaaaagaggcgtatcttagttcaccggaaaagcattttggaaggagtaaaaggtttcttgcttcattagaggagaaaaatggttatgttaaggcagataaagtcatatcagaagatatcatagaggaagctaagatgttagctacttgtgcatatgaggatggcacacattggggtcaagagattggttattcatacgattgtcatttggagagcacttttactggttatctattacactgcaaagggtggacatctacttatttgtatccagacaggccatctttcttgggttgtgccccagttgatatgcaaggtttctcatcacagctcatcaaatgggttgctgcacttacacaagctggtttatcacatctcaatcccatcacttatggtttgagtagtaggatgaggactctccaatgcatgtgctatgcctatttgatgtatttcactctttattcttggggaatggttatgtatgctagtgttccttctattggccttttgtttgacttccaagtctatcctgaggtacatgatccgtggtttgcagtgtatgtgattgctttcatatcgacaattttggagaatatgtcggagtcaattccagaagggggatcagttaaaacgtggtggatggaatacagggcattgatgatgatgggagttagcgcaatatggttaggaggattgaaagctatatatgacaagatagtcggaacacaaggagagaaattgtatttgtcggacaaggcaattgacaaggaaaagctcaagaaatacgagaagggcaaatttgatttccaaggaatagggatacttgctctgccactgatagcattttccgtgttgaacctcgtaggcttcattgttggagctaatcatgtctttattactatgaactacgcaggcgtgctgggccaactcctcgtatcatcgttcttcgtctttgttgtcgtcactgttgtcattgatgttgtatctttcttaaaggtttcttaa

SEQ ID NO: 31 - protein, 713 aa, Solanum lycopersicum
MKKTMELNKSTVPQPITTVYRLHMFIHSIIMLALIYYRVSNLFKFENILSLQALAWALITFGEFSFILKWFFGQGTRWRPVERDVFPENITCKDSDLPPIDVMVFTANPKKEPIVDVMNTVISAMALDYPTDKLAVYLADDGGCPLSLYAMEQACLFAKLWLPFCRNYGIKTRCPKAFFSPLGDDDRVLKNDDFAAEMKEIKLKYEEFQQKVEHAGESGKINGNVVPDRASLIKVINERENEKSVDDMTKMPLLVYVSRERRFNRLHHFKGGSANALLRVSGIMSNAPYVLVLDCDFFCHDPISARKAMCFHLDPKLSSDLAYVQFPQVFYNVSKSDIYDVKIRQAYKTIWHGMDGIQGPVLSGTGYFLKRKALYTSPGVKEAYLSSPEKHFGRSKRFLASLEEKNGYVKADKVISEDIIEEAKMLATCAYEDGTHWGQEIGYSYDCHLESTFTGYLLHCKGWTSTYLYPDRPSFLGCAPVDMQGFSSQLIKWVAALTQAGLSHLNPITYGLSSRMRTLQCMCYAYLMYFTLYSWGMVMYASVPSIGLLFDFQVYPEVHDPWFAVYVIAFISTILENMSESIPEGGSVKTWWMEYRALMMMGVSAIWLGGLKAIYDKIVGTQGEKLYLSDKAIDKEKLKKYEKGKFDFQGIGILALPLIAFSVLNLVGFIVGANHVFITMNYAGVLGQLLVSSFFVFVVVTVVIDVVSFLKVS

SEQ ID NO: 32 - DNA, 2142 bp, Solanum pennellii
  source 1..2142: mol_type = unassigned DNA
atgaaaaaaaccatggagctcaacaaaagcactgttccacaacctatcaccaccgtataccgactccacatgttcatccactcaataatcatgcttgcattaatatactaccgtgtatctaatttgtttaaattcgaaaacattctcagtttacaagcacttgcttggctactcatcacttttggtgaatttagtttcattctcaagtggttcttcggacaaggaactcgttggcgccccgttgaacgagatgttttccctgaaaacattacttgcaaagattccgatctaccgccaattgacgtaatggtgttcactgccaatcctaagaaagagccaattgtagatgtcatgaacactgtgatatccgcaatggctcttgattatcccaccgataaattggctgtgtatctggccgatgatggaggatgtccattgtccttgtacgccatggaacaagcatgtttgtttgcaaagctatggttacctttctgtagaaagtatggaattaaaacgagatgcccaaaagcatttttttctccgttaggagatgatgaccgtgttcttaagaatgatgattttgctgctgaaatgaaagaaattaaattgaaatatgaagagttccagcagaacgtggaacatgctggtgaatctggaaaaatcaatggcaacgtagtgcctgacagagcttcgcttattaaggtaataaacgagagggagaacgaaaagagtgtcgatgatttaacgaaaatgcccttgctagtttatgtatcccgtgaaagaagattcaaccgtcttcatcatttcaagggtggatctgcaaatgctctacttcgagtttctggaataatgagtaatgccccctatgtactggtgttagattgtgatttcttctgtcatgatccgatatcagctaggaaagcaatgtgttttcatcttgatccaaagctatcatctgatttagcctatgttcagttccctcaagtcttttacaatgtcagcaagtccgatatttatgatgtcaaaattagacaggcttacaagacaatatggcatggaatggatggtatccaaggcccagtgttatctggaactggttattttctcaagaggaaggcgttatacacaagtccaggagtaaaagaggcgtatcttagttcaccggaaaagcattttggaaggagtaaaaagttccttgcttcattagaggagaaaaatggttatgttaaggcagataaagtcatatcagaagatatcatagaggaagctaagatcttagctacttgtgcatatgaggatggcacacattggggtcaagagattggttattcatacgattgtcatttggagagcacttttactggttatctattacactgcaaagggtggacatctacttatttgtatccagacaggccatctttcttgggttgtgccccagttgatatgcaaggtttctcatcacagctcataaaatgggttgctgcacttacacaagctggtctatcacatctcaatcccatcacttatggtttgagtagtaggatgagaactctccaatgcatgtgctatgcctatttgatgtatttcactctttattcttggggaatggttatgtatgctagtgttccttctattggccttttgtttggcttccaagtctaccctgaggtacatgatccatggtttgcagtgtatgtgattgctttcatatcgacaattttggagaatatgtcggagtcaattccagaagggggatcagttaaaacgtggtggatggaatacagggcattgatgatgatgggagttagcgcaatatggttaggaggattgaaagctatatatgacaagatagtcggaacacaaggagagaaattgtatttgtcggacaaggcaattgacaaggaaaagctcaagaaatacgagaagggcaaatttgatttccaaggaatagggatacttgctctgccattgatagcattttccgtgttgaacctcgtaggcttcattgttggagctaatcatgtctttattactatgaactacgcaggcgtgctgggccaactcctcgtatcatcattcttcgtctttgttgtcgtcactgttgtcattgatgttgtatctttcttaaaggtttcttaa

SEQ ID NO: 33 - protein, 713 aa, Solanum pennellii
MKKTMELNKSTVPQPITTVYRLHMFIHSIIMLALIYYRVSNLFKFENILSLQALAWLLITFGEFSFILKWFFGQGTRWRPVERDVFPENITCKDSDLPPIDVMVFTANPKKEPIVDVMNTVISAMALDYPTDKLAVYLADDGGCPLSLYAMEQACLFAKLWLPFCRKYGIKTRCPKAFFSPLGDDDRVLKNDDFAAEMKEIKLKYEEFQQNVEHAGESGKINGNVVPDRASLIKVINERENEKSVDDLTKMPLLVYVSRERRFNRLHHFKGGSANALLRVSGIMSNAPYVLVLDCDFFCHDPISARKAMCFHLDPKLSSDLAYVQFPQVFYNVSKSDIYDVKIRQAYKTIWHGMDGIQGPVLSGTGYFLKRKALYTSPGVKEAYLSSPEKHFGRSKKFLASLEEKNGYVKADKVISEDIIEEAKILATCAYEDGTHWGQEIGYSYDCHLESTFTGYLLHCKGWTSTYLYPDRPSFLGCAPVDMQGFSSQLIKWVAALTQAGLSHLNPITYGLSSRMRTLQCMCYAYLMYFTLYSWGMVMYASVPSIGLLFGFQVYPEVHDPWFAVYVIAFISTILENMSESIPEGGSVKTWWMEYRALMMMGVSAIWLGGLKAIYDKIVGTQGEKLYLSDKAIDKEKLKKYEKGKFDFQGIGILALPLIAFSVLNLVGFIVGANHVFITMNYAGVLGQLLVSSFFVFVVVTVVIDVVSFLKVS

SEQ ID NO: 34 - DNA, 2148 bp, Solanum tuberosum
  source 1..2148: mol_type = unassigned DNA
atgaaaaaaaccatggagctcaacaaaagcactgttccacaacctatcaccaccatataccgactccacatgtttatccactctataatcatggttgcattaatatactaccgtgtatctaatttgtttaaattcgaaaacattctgagtttacaagcacttgcttgggtactcatcacttttggtgaatttagtttcattctcaagtggttcttcggacaaggaactcgttatcgccctgttgaaagagatgttttccctgaaaacataacttgcaaagattccgatctaccaccaattgacgtaatggtattcactgccaatcctaagaaagagccaattgtggatgtcatgaacactgtgatatccgcaatggctcttgattatcctacggataaattggctgtgtatctggctgatgatggaggatgtcctttgtcattgtacgccatggaagaagcatgtgtgtttgcaaagctgtggctacctttctgtaggaagtatggaattaaaactagatgccctaaagcgtttttttctcctttaggagatgatgaacgtgttcttaagaatgatgattttgatgctgaaatgaaagaaattaaattgaaatatgaagagttccagcagaatgtggaacgtgctggtgaatctggaaaaatcaatggtaacgtagtgcctgatagagcctcgtttattaaggtaataaacgacagaaaagcggagagcgaaaagagtgccgatgatttaacgaaaatgcccttgctagtttatgtatcccgtgaaagaagattcaaccgtcttcatcacttcaagggtggatctgcaaatgctcttcttcgagtttctggaataatgagtaatgccccctatatactggtgttagattgtgatttcttctgtcatgatccaatatcagctaggaaggcaatgtgttttcatcttgatccaaagctatcatctgatttagcttatgttcagttccctcaagtcttttacaatgtcagcaagtccgatatttatgatgtcaaaattagacaggcttacaagacaatatggcatggaatggatggtatccaaggcccagtgttatcaggaactggttattttctgaagaggaaggcgttatacacgagtccaggagtaaaggaggagtatcttagttcaccggaaaagcattttggaaggagtaaaaagttccttgcttcactagaggagaaaaatggttatgttaaggcagagaaagtcatatcagaagatatcgtagaggaagctaagaccttagctacttgtgcatatgaggatggcacacattggggtcaagagattggttattcatacgattgtcatttggagagcacttttactggttatctattacactgcaaagggtggagatcgacttatttgtatccagacaggccatctttcttgggttgtgccccagttgatatgcaaggtttctcctcacagctcataaaatgggttgctgcacttacacaagctggtttatcacatctcaatcccatcacttatggctttagtagcaggatgaaaactctccaatgcatgtgctatgcctatttgatatatttcactctttattcttggggaatggttctatatgctagtgttccttctattggccttttgtttggcttccaagtctatcccgatgtacatgatccatggtttgcagtgtatgtgattgctttcatatcggcaattttggagaatatgtcggagtcaattcctgatgggggatcatttaaatcttggtggatggaatacagggcactgatgatgatgggagttagtgcaatatggttaggaggattgaaagctatattagacaggataatcggaacagaaggagagaaattgtatttatcggacaaggcaattgacaaggaaaagctcaagaaatacgagaaggggaaatttgatttccaaggaatagggatacttgctgtaccattgatagcattttccttgttgaacctcgtaggcttcattgttggagctaatcatgtctttattactatgaactacgcaggtgtgcttggccaactcctcgtatcatccttcttcgtctttgtcgtggtcactgttgtcattgatgtcgtttctttcttaaaggtttcttaa

SEQ ID NO: 35 - protein, 711 aa, Solanum tuberosum
MELNKSTVPQPITTIYRLHMFIHSIIMVALIYYRVSNLFKFENILSLQALAWVLITFGEFSFILKWFFGQGTRYRPVERDVFPENITCKDSDLPPIDVMVFTANPKKEPIVDVMNTVISAMALDYPTDKLAVYLADDGGCPLSLYAMEEACVFAKLWLPFCRKYGIKTRCPKAFFSPLGDDERVLKNDDFDAEMKEIKLKYEEFQQNVERAGESGKINGNVVPDRASFIKVINDRKAESEKSADDLTKMPLLVYVSRERRFNRLHHFKGGSANALLRVSGIMSNAPYILVLDCDFFCHDPISARKAMCFHLDPKLSSDLAYVQFPQVFYNVSKSDIYDVKIRQAYKTIWHGMDGIQGPVLSGTGYFLKRKALYTSPGVKEEYLSSPEKHFGRSKKFLASLEEKNGYVKAEKVISEDIVEEAKTLATCAYEDGTHWGQEIGYSYDCHLESTFTGYLLHCKGWRSTYLYPDRPSFLGCAPVDMQGFSSQLIKWVAALTQAGLSHLNPITYGFSSRMKTLQCMCYAYLIYFTLYSWGMVLYASVPSIGLLFGFQVYPDVHDPWFAVYVIAFISAILENMSESIPDGGSFKSWWMEYRALMMMGVSAIWLGGLKAILDRIIGTEGEKLYLSDKAIDKEKLKKYEKGKFDFQGIGILAVPLIAFSLLNLVGFIVGANHVFITMNYAGVLGQLLVSSFFVFVVVTVVIDVVSFLKVS

SEQ ID NO: 36 - DNA, 2148 bp, Solanum chacoense
  source 1..2148: mol_type = unassigned DNA
atgaaaaaaaccatggagctcaacaaaagcactgttccacaacctatcaccaccatataccgactccacatgttcgtccattctataatcatggctgcattaatatactaccgtgtatctaatttgtttaaattcgaaaacattctgagtttacaagcacttgcttgggtactcatcacttttggtgaatttagtttcattctcaagtggttcttcggacaaggaactcgttggcgccctgttgaaagagatgttttccctgaaaacataacttgcaaagattccgatctaccaccaattgacgtaatggtattcactgccaatcctaagaaagagccaattgtggatgtcatgaacactgtgatatccgcaatggctctagattatcctacggataaattggctgtgtatctggctgatgatggaggatgtcctttgtcattgtacgccatggaagaagcatgtgtgtttgcaaagctgtggctacctttctgtaggaagtatggaattaaaaccagatgccctaaagcgtttttttctcctttaggagatgatgaccgtgttcttaagaatgatgattttgatgctgaaatgaaagaaattaaattgaaatatgaagagttccagcagaatgtggaacgtgctggtgaatctggaaaaatcaatggtaacgtagtgcctgatagagcctcgtttattaaggtaataaacgacagaaaaacggagagcgaaaagagtgccgatgatttaacgaaaatgcccttgctagtttatgtatcccgtgaaagaagattcaaccgtcttcatcacttcaagggtggatctgcaaatgctcttcttcgagtttctggaataatgagtaatgccccctatatactggtgttagattgtgatttcttctgtcatgatccaatatcagctaggaaggcaatgtgttttcatcttgatccaaagctatcatctgatttagcttatgttcagttccctcaagtcttttacaatgtcagcaagtccgatatttatgatgtcaaaattagacaggcttacaagacaatatggcatggaatggatggtatccaaggcccagtgttatcaggaactggttattttctgaagaggaaggcgttatacacgagtccaggagtaaaggaggagtatcttagttcaccggaaaagcattttggaaggagtaaaaagttccttgcttcactagaggagaaaaatggttatgttaaggcagagaaagtcatatcagaagatatcgtagaggaagctaagaccttagctacttgtgcatatgaggatggtacacattggggtcaagagatcggttattcatacgattgtcatttggagagcacttttactggttatctattacactgcaaagggtggacatcgacttatttgtatccagacaggccatctttcttgggttgtgctccagttgatatgcaaggtttctcctcacagctcataaaatgggttgctgcacttacacaagctggtttatcacatctcaatcccatcacttatggcttgagtagcaggatgaaaactctccaatgcatgtgctatgcctatttgatatatttcactctttattcttggggaatggttctatatgctagtattccttctattggtcttttgtttggcttccaagtctatccggaggtacatgatccatggtttgcagtgtatgtgattgctttcatatcgacaattttggagaatatgtcggagtcaattccagaagggggatcatttaaatcgtggtggatggaatacagggcactgatgatgatgggagttagtgcaatatggttaggaggattgaaagctatattagacaagataatcggaacagaaggagagaaattgtatttgtcagacaaggcaattgacaaggaaaagctcaagaaatacgagaaggggaaatttgatttccaaggaatagggatacttgctgtaccattgatagcattttccctgttgaacctggtaggcttcattgttggagctaatcatgtctttattactatgaactacgcaggtgtgcttggccaactcctcgtatcatccttcttcgtctttgtcgtggtcactgttgtcattgatgtcgtttctttcttaaaggtttcttaa

SEQ ID NO: 37 - protein, 715 aa, Solanum chacoense
MKKTMELNKSTVPQPITTIYRLHMFVHSIIMAALIYYRVSNLFKFENILSLQALAWVLITFGEFSFILKWFFGQGTRWRPVERDVFPENITCKDSDLPPIDVMVFTANPKKEPIVDVMNTVISAMALDYPTDKLAVYLADDGGCPLSLYAMEEACVFAKLWLPFCRKYGIKTRCPKAFFSPLGDDDRVLKNDDFDAEMKEIKLKYEEFQQNVERAGESGKINGNVVPDRASFIKVINDRKTESEKSADDLTKMPLLVYVSRERRFNRLHHFKGGSANALLRVSGIMSNAPYILVLDCDFFCHDPISARKAMCFHLDPKLSSDLAYVQFPQVFYNVSKSDIYDVKIRQAYKTIWHGMDGIQGPVLSGTGYFLKRKALYTSPGVKEEYLSSPEKHFGRSKKFLASLEEKNGYVKAEKVISEDIVEEAKTLATCAYEDGTHWGQEIGYSYDCHLESTFTGYLLHCKGWTSTYLYPDRPSFLGCAPVDMQGFSSQLIKWVAALTQAGLSHLNPITYGLSSRMKTLQCMCYAYLIYFTLYSWGMVLYASIPSIGLLFGFQVYPEVHDPWFAVYVIAFISTILENMSESIPEGGSFKSWWMEYRALMMMGVSAIWLGGLKAILDKIIGTEGEKLYLSDKAIDKEKLKKYEKGKFDFQGIGILAVPLIAFSLLNLVGFIVGANHVFITMNYAGVLGQLLVSSFFVFVVVTVVIDVVSFLKVS

SEQ ID NO: 38 - DNA, 2058 bp, Solanum melongena
  source 1..2058: mol_type = unassigned DNA
atgaaaaaacaaatggagctcaacagaagtgttgtaccgcaacctatcaccaccatttaccgtctccacatgtttatccatgccctaatcatgctagcactaatatactaccgtgtctctaatttggccaaattcgaaaacatcctcagtttacaagcacttgcttgggctcttatcacgttaggtgaactttgtttcatagtcaagtggttcttcggacaagggactcgttggcgtcctgttgatagggatgtcttccctgaaaacatcacttgtccagattccgagctaccccccattgatgtcatggttttcactgcaaatcctaagaaagagccaattgtggatgtcatgaacactgtcatatccgcaatggctcttgattacccgaccgacaaattggccgtttatttgtctgatgatggaggatgccccttgacgttgtacgcaatggaggaagcttgttcctttgccaagttgtggctacctttttgtaggaagtatggaatcaaaacaaggtgccctaaggcgtttttttctccattaggagaagatgaccgtgtattgaagagtgatgactttgtttctgaaatgaaagaaatgaagtcaaaatatgaagagttccagcagaacgtggaccgtgctggtgaatccggaaaaatcaaaggtgacgtagtgcctgatagacccgcgtttcttaaggtactaaatgacaggaagacggagaacgagaagagtgcagacgatttaactaaaatgcctttgctagtatacgtatcccgtgaaagaagaactcaccgtcgccatcacttcaagggtggatctgcaaatgctcttcttcgagtttctgggataatcagtaatgccccctatatactggttttagattgtgatttcttctgtcatgatccaatatcagctcggaaggcaatgtgtttccatcttgatccaaaactatcacctgacttagcttacgtgcagttccctcaagtgttttacaatgttagcaagtccgatatttacgacgtcaaaattagacaggcttacaagacaatatggcacgggatggatggtatccaaggcccagtgttatcgggaactggttattttttaaaaaagaaggcgttgtacacgagtccaggtctaaaagatgagtatcttagttcaccggaaaagcatttcggaacgagtagaaagttcattgcttcactagaggagaataattatgttaagcaagagaaagtcatatcagaagatatcatagaggaagctaagagactggctacttgtgcatacgaggatggcacacattggggtcaagaggcaaacaggccatctttcttgggttgtgccccagttgatatgcaaggtttctcctcacagctcataaaatgggttgctgcactcacacaagcaggtctatcacatctcaatcccatcacttacggcttcaagagcagaatgagaactctccaagtcttgtgttatgcctatttgatgtatttctctctttattcttggggaatggttctacatgctagtgttccttctattggccttctctctggcattaaaatctacccggaggtgtatgatccatggtttgttgtgtatgtgattgctttcatatcaacaattttggagaatatgtcggaatcaattccggaagggggatcggttaaaacgtggtggatggaatacagggcactgatgatgatgggagttagtgcaatatggctaggaggagtgaaagccatagtagacaagatcatcggaacgcaaggagagaaattgtatttgtcggacaaagcaattgacaaggaaaagctcaagaaatacgagaaggggaaatttgatttccaaggaataggaatacttgctgtaccattgataacattttctgtgttgaacctggtaggcttcttggttggaattaatcaagtgttgataacgatgaagttcgcaggcgtgctgggccaactcctcgtatcatccttcttcgtctttgtcgtcgttactgttgtcattgatgtcgtatctttcttaaaggattcttaa

SEQ ID NO: 39 - protein, 685 aa, Solanum melongena
MKKQMELNRSVVPQPITTIYRLHMFIHALIMLALIYYRVSNLAKFENILSLQALAWALITLGELCFIVKWFFGQGTRWRPVDRDVFPENITCPDSELPPIDVMVFTANPKKEPIVDVMNTVISAMALDYPTDKLAVYLSDDGGCPLTLYAMEEACSFAKLWLPFCRKYGIKTRCPKAFFSPLGEDDRVLKSDDFVSEMKEMKSKYEEFQQNVDRAGESGKIKGDVVPDRPAFLKVLNDRKTENEKSADDLTKMPLLVYVSRERRTHRRHHFKGGSANALLRVSGIISNAPYILVLDCDFFCHDPISARKAMCFHLDPKLSPDLAYVQFPQVFYNVSKSDIYDVKIRQAYKTIWHGMDGIQGPVLSGTGYFLKKKALYTSPGLKDEYLSSPEKHFGTSRKFIASLEENNYVKQEKVISEDIIEEAKRLATCAYEDGTHWGQEANRPSFLGCAPVDMQGFSSQLIKWVAALTQAGLSHLNPITYGFKSRMRTLQVLCYAYLMYFSLYSWGMVLHASVPSIGLLSGIKIYPEVYDPWFVVYVIAFISTILENMSESIPEGGSVKTWWMEYRALMMMGVSAIWLGGVKAIVDKIIGTQGEKLYLSDKAIDKEKLKKYEKGKFDFQGIGILAVPLITFSVLNLVGFLVGINQVLITMKFAGVLGQLLVSSFFVFVVVTVVIDVVSFLKDS

SEQ ID NO: 40 - DNA, 2136 bp, Capsicum annuum
  source 1..2136: mol_type = unassigned DNA
atggagctcaacagatgtacggtgcagcaacctaccactgccatataccgactacacatgtttctccactctctaatcatgcttgcattagtatactatcgtttgtctaatctgttttacttcgaaaacgtcctcactttacaagcatttgcatgggggcttatcaccttaggtgaaatttgtttcattgtcaagtggttctttggtcaagggactcgttggcgccccgttgtcagggaagtgttcctggacaatattacttgccaagattccgagctgcccgcactagatgtgatggttttcactgccaatcccaagaaagagccaattgtggatgtcatgaacactgtgatatccgcaatggctcttgattacccgacggataaattggctgtgtatctggctgatgatggaggatgccccttgacgttgtacgccatggaggaggcctgttcttttgccaagttgtggctacctttctgtaggaagtatggaatcaaaacaaggtgccccaaagcgtttttttctccattaggagaagatgatcgtatccttaagaacgatgactttgtagctgaaatgaaagaaattaaattaaaatatgaggagttccagcagaatgtaaaccttgctggtgaatccggaaaaatcaaaggtgacgtagtgcctgatagagcctcgtttattaaggtaataaatgacaggaaaatggagaacaagaagagtgccgacgatataacgaaaatgcctttgctagtatacgtatcccgtgaaagaagatttaacagtcgtcatcacttcaagggtggatctgcaaatgctcttcttcgagtttcagggataatgagtaatgccccctatttactggtcttagattgtgatttcttctgtcatgatccaacatcagctcggaaggcaatgtgtttccatcttgatccaaaactatcaccttccttagcttatgtgcagttccctcaagtgttttacaatgtcagcaagtccgatatatacgatgtcaaaattagacaggcttacaagacaatatggcacggaatggatggtatccaaggcccagtgttatcgggaactgggtattttctgaagaggaaagcgttatacacgagtccaggtctaaaggatgagtatcttatttcaccggaaaagcatttcggatcaagtagaaagttcattgcttctctagaggagaacaatggttatgttaagcaagagaaactcataacagaagatattatagaggaagcgaagaccttgtctacttgtgcatacgaggatggtacacgatggggcgaagagatcggttatacctacaattgccatttggagagcacttttaccggctatcttttgcactgcaaagggtggacatcaacatatttgtatccagaaaggccatctttcttgggttgtgccccagttgatatgcaaggattctcctcacaactcacaaaatgggttgctgcactcacacaagctggtctatcacatctcaatcccatcacttacggcatgaagagcaggattaagactatccaatgcttgtgctatgcctatttgatgtatttctctctctattcttggggaatggttctgcatgctagtgttccttctattagccttttgcttggcattcaagtctaccccgaggtctatgatccatggtttgcagtgtatgtgcttgctttcatatcgacaattttggagaacatgtcagagtcaattccagaaggcggttcagttaaaacttggtggatggaatacagggcactgatgatgatgggagttagtgcaatatggttaggaggagtgaaagctatagtagaaaagatcatcggaactcaaggagagaaattatatttgtcggacaaagcaattgacaaggaaaagctcaagaaatatgagaaggggaaatttgatttccaagggatagggatacttgctgttccattgataacattctcagcgttgaatttggtaggcttcatggttggagctaatcaagtgattcttactatgaagttcgaagctttgctaggccaactccttgtgtcatccttcttcgtctttgtggtggtcaccgttgtcatagatgtcctatctttcttaaaagactcttaa

SEQ ID NO: 41 - protein, 711 aa, Capsicum annuum
MELNRCTVQQPTTAIYRLHMFLHSLIMLALVYYRLSNLFYFENVLTLQAFAWGLITLGEICFIVKWFFGQGTRWRPVVREVFLDNITCQDSELPALDVMVFTANPKKEPIVDVMNTVISAMALDYPTDKLAVYLADDGGCPLTLYAMEEACSFAKLWLPFCRKYGIKTRCPKAFFSPLGEDDRILKNDDFVAEMKEIKLKYEEFQQNVNLAGESGKIKGDVVPDRASFIKVINDRKMENKKSADDITKMPLLVYVSRERRFNSRHHFKGGSANALLRVSGIMSNAPYLLVLDCDFFCHDPTSARKAMCFHLDPKLSPSLAYVQFPQVFYNVSKSDIYDVKIRQAYKTIWHGMDGIQGPVLSGTGYFLKRKALYTSPGLKDEYLISPEKHFGSSRKFIASLEENNGYVKQEKLITEDIIEEAKTLSTCAYEDGTRWGEEIGYTYNCHLESTFTGYLLHCKGWTSTYLYPERPSFLGCAPVDMQGFSSQLTKWVAALTQAGLSHLNPITYGMKSRIKTIQCLCYAYLMYFSLYSWGMVLHASVPSISLLLGIQVYPEVYDPWFAVYVLAFISTILENMSESIPEGGSVKTWWMEYRALMMMGVSAIWLGGVKAIVEKIIGTQGEKLYLSDKAIDKEKLKKYEKGKFDFQGIGILAVPLITFSALNLVGFMVGANQVILTMKFEALLGQLLVSSFFVFVVVTVVIDVLSFLKDS

SEQ ID NO: 42 - DNA, 270 bp, synthetic construct
  misc_feature 1..270: note = Syn. Polynucl.
  source 1..270: mol_type = other DNA
ggctcttgattatcccaccgataaattggctgtgtatctcgctgatgatggaggatgtccattgtcgttgtacgccatggaacaagcgtgtttgtttgcaaagctatggttacctttctgtagaaactatggaattaaaacgagatgcccaaaagcatttttttctccgttaggagatgatgaccgtgttcttaagaatgatgattttgctgctgaaatgaaagaaattaaattgaaatatgaagagttccagcagaaggtggaacatgc

SEQ ID NO: 43 - DNA, 275 bp, synthetic construct
  misc_feature 1..275: note = Syn. Polynucl.
  source 1..275: mol_type = other DNA
ggctcttgattatcctacggataaattggctgtgtatctggctgatgatggaggatgtcctttgtcattgtacgccatggaagaagcatgtgtgtttgcaaagctgtggctacctttctgtaggaagtatggaattaaaactagatgccctaaagcgtttttttctcctttaggagatgatgaacgtgttcttaagaatgatgattttgatgctgaaatgaaagaaattaaattgaaatatgaagagttccagcagaatgtggaacgtgctggtg

SEQ ID NO: 44 - DNA, 447 bp, synthetic construct
  misc_feature 1..447: note = Syn. Polynucl.
  source 1..447: mol_type = other DNA
ggctcttgattacccgaccgacaaattggccgtttatttgtctgatgatggaggatgccccttgacgttgtacgcaatggaggaagcttgttcctttgccaagttgtggctacctttttgtaggaagtatggaatcaaaacaaggtgccctaaggcgtttttttctccattaggagaagatgaccgtgtattgaagagtgatgactttgtttctgaaatgaaagaaatgaagtcaaaatatgaagagttccagcagaacgtggaccgtgctggtgaatccggaaaaatcaaaggtgacgtagtgcctgatagacccgcgtttcttaaggtactaaatgacaggaagacggagaacgagaagagtgcagacgatttaactaaaatgcctttgctagtatacgtatcccgtgaaagaagaactcaccgtcgccatcacttcaagggtgg

SEQ ID NO: 45 - DNA, 2292 bp, Spinacia oleracea
  source 1..2292: mol_type = unassigned DNA
atgtggaggttgaaggttggagaaggggctaatgacccatacttatatagcactaataactttgttgggcgtcaaacttgggagtttgatcctaactatggcacccctgaggatattcaagaggtcgaagatgctcgccgcgatttttacaataatcggtttaaagtgaagccttgtaacgatctcttatggcgttttcagttcttaagagagaaaaacttcaagcaaaccatacctcaagtgaaggtgggtgacggggaggagatcacatatgagaccgcctcgacgacattaaagagagcggtgaatattttcacagccttgcagtctgaacatggccattggccggctgaaattgctggccctcagttcttccttcctcctttggtattttgcttatacattacaggagatcttaactctgttttcggaccagaacatcgtagagaaattcttcgcagcatctactatcaccagaacgaagatggaggttggggattacatattgaaggacatagcaccatgttctgtaccgcactgaattacatatgtttacgaatgcttggaataggacctgatgaaggtgatgacaacgcgtgccctagagcgcgtaaatggattctcgaccatggtagcgttacacatatcccttcttggggtaaaacttggttatctatactgggcctgttcgattggtctggaagtaacccaatgccacctgagttctggatccttcctacttttctccctatgcatccagcaaaaatgtggtgctactgtcgaatggtgtatatgccaatgtcatacttgtatgggaagagattcgtaggtccaatcacacctctcattaaacaacttagggaagaactctacaacgaaccctttgaacaaattagttggaagaaaatgcgacatttgtgtgcaccggaggatctctactatcctcatccattgattcaagacttgatgtgggacgctctttacctttttacggaacctctcctgacccgttggcctttcaacaagttgatacgaaagaaagcattagaggttacaatggaacacatacattatgaagatgagaacagtcgttacataacaattggatgtgtcgagaaggttttatgtatgttagcctgttgggtggaagaccctaaaggggatcattacaagaaacatcttgcaagagtacaagattacatttggattgctgaagatggattgaaaatgcagagttttggaagtcaacaatgggattgtgggttttcagtacaggcattattagcttctaatcttagtctcgacgaaattggacctgctcttaagaaaggccatttcttcattaaggagtcacaggtgaaggacaatccatccggcgacttcaaagctatgcatcgccatatctcaaagggatcgtggactttctccgaccaagatcatggttggcaagtctccgattgcactgccgaaggccttaagtgttgtctaatcttatcaacaatgcccccggaaattgttggagaaaagatggaccctgaacgcctttatgattctgtcaatgtcttgctttctctacagagtaataaaggagggctagctgcctgggaaccagcaggggctcaagaatggttggaggtcctaaacccaacagaattctttgaagacattgtgattgaacatgagtatgtagagtgtacggcttcagcaattcaagctttaataatgttcaagaagttatacccaggacacaggaaaaaagagattgaaaattttgtagtaaacgcagtcaagtaccttgaaaacacccaatatcctagtggaggatggtatggaaattgggggatttgtttcatatatggaacatggtttgcactaggagggctagcagcaggtgggaagacatactataattgtgctgctgttaggaagggtgttgattttttgcttactacacaaaaggaggatggtggttggggtgaaagttatatttcttgtcccaataaggaatttgtgccaatagagggaaagtccaatttggttcagactggttgggctttgatgggtctacttcatgctggacaggcggagagggatccaactcctctgcatcgtgcagcaaagcttttgattaattcacaactcgaaaatggcgatttccctcaacaggaaataacaggagtcttcatgaagaattgcatgttacattatccgatgtacagaagcatttatccactgtgggcaattgcagaatacagaaagcgtgtttcattaccttctatcaactctacttga

SEQ ID NO: 46 - DNA, 1452 bp, Spinacia oleracea
  source 1..1452: mol_type = unassigned DNA
atggaactcttctttatgtgtgggctagtccttttcctctccctatctctagcctccttcttccttttctataaccaccatagaacccgggggtacaagctacccccgggcaagatggggtggccggtggtgggcgagtcatttgaattttttcaaaccgggtggaaaggttacccggaaaagttcatatttgatagactgaacaagtacaccccaagccaagtgttcaagacttccatcgtaggagaaaaggttgcggttttatgtggcgcggcgggtaacaagttcttgtactcaaacgagaacaagttagtacaagcttggtggcctagctctgttgataagatctttccttcttctacccaaacttcctccaaagaagaggctaagaagatgcggaaactcctccctaacttcctcaagcccgaggctttacataggtacatacccatcatggatagcattgccatccggcacatggagtccgggtgggagggaaaggacaaggtagaagtcttccctttggctaagaattacaccttctggctggcttgccgactcttcttaagcgtcgaggacccggctcatgtagccaagttctccgaaccattcaacgacatagccgcagggatcatctcgatgccaatcgacctccccggaacacccttcaaccgagggatcaagtcgtctaacgtcgtaaggaaagagttgagggccatcataaagcagaggaaacttgacttagcagatggcaaggcttcacctacacaagatattctgtctcatatgttgttgacttgtactgaagatggcaagtttatgagtgaaatggatattgctgataagattctgggacttcttattggtggacatgatactgctagtgcttcttgtacttttgttgttaagtttcttgctgagcttcctcacatatatgaaggtgtctacaaagagcaaatggagatagcaaattcaaaaaaagcaggagaacttctaaattgggaggacatacaaaaaatgaaatactcatggaatgtagcttgtgaagttatgcgtttggctcctccacttcaaggtggtttcagggaagccctttctgatttcatgtataacggattccaaatccccaagggctggaagttatattggagtgcaaattcaacacatatgaacccggaatgcttcccggagcccaagacgttcgacccatcgaggttcgacggtacgggaccagcaccatacacatacgtccccttcggaggaggaccgagaatgtgcccgggcaaggagtatgcaaggctagagatattagtgttcatgcacaacgttgtcaagaggtttaaatgggaaaaaatgcttcctgatgagaaggttattgtcaatcccatgcctatcccagaacatggccttcctgtccgccttttccctcatcctcgaactgtagctgcttaa

SEQ ID NO: 47 - DNA, 279 bp, Spinacia oleracea
  source 1..279: mol_type = unassigned DNA
atggagttcttctttctgtgtggtctagtcttttacttctccatatctctagcctccttcttccttttctataaccaccatacaacccgggtttacccgctacccgcgggcgagatggggtggccggtggtgggcgactcgtttgaattttttcaaaccgggtggaacggttacccggaaaatttcatctttgatagactcaacaaatacaccccaagccaagtgttcaagactttcatcctaagagaaaaggttgtgttttatttgagaaggttgtag

SEQ ID NO: 48 - protein, 483 aa, Spinacia oleracea
MELFFMCGLVLFLSLSLASFFLFYNHHRTRGYKLPPGKMGWPVVGESFEFFQTGWKGYPEKFIFDRLNKYTPSQVFKTSIVGEKVAVLCGAAGNKFLYSNENKLVQAWWPSSVDKIFPSSTQTSSKEEAKKMRKLLPNFLKPEALHRYIPIMDSIAIRHMESGWEGKDKVEVFPLAKNYTFWLACRLFLSVEDPAHVAKFSEPFNDIAAGIISMPIDLPGTPFNRGIKSSNVVRKELRAIIKQRKLDLADGKASPTQDILSHMLLTCTEDGKFMSEMDIADKILGLLIGGHDTASASCTFVVKFLAELPHIYEGVYKEQMEIANSKKAGELLNWEDIQKMKYSWNVACEVMRLAPPLQGGFREALSDFMYNGFQIPKGWKLYWSANSTHMNPECFPEPKTFDPSRFDGTGPAPYTYVPFGGGPRMCPGKEYARLEILVFMHNVVKRFKWEKMLPDEKVIVNPMPIPEHGLPVRLFPHPRTVAA

SEQ ID NO: 49 - protein, 763 aa, Spinacia oleracea
MWRLKVGEGANDPYLYSTNNFVGRQTWEFDPNYGTPEDIQEVEDARRDFYNNRFKVKPCNDLLWRFQFLREKNFKQTIPQVKVGDGEEITYETASTTLKRAVNIFTALQSEHGHWPAEIAGPQFFLPPLVFCLYITGDLNSVFGPEHRREILRSIYYHQNEDGGWGLHIEGHSTMFCTALNYICLRMLGIGPDEGDDNACPRARKWILDHGSVTHIPSWGKTWLSILGLFDWSGSNPMPPEFWILPTFLPMHPAKMWCYCRMVYMPMSYLYGKRFVGPITPLIKQLREELYNEPFEQISWKKMRHLCAPEDLYYPHPLIQDLMWDALYLFTEPLLTRWPFNKLIRKKALEVTMEHIHYEDENSRYITIGCVEKVLCMLACWVEDPKGDHYKKHLARVQDYIWIAEDGLKMQSFGSQQWDCGFSVQALLASNLSLDEIGPALKKGHFFIKESQVKDNPSGDFKAMHRHISKGSWTFSDQDHGWQVSDCTAEGLKCCLILSTMPPEIVGEKMDPERLYDSVNVLLSLQSNKGGLAAWEPAGAQEWLEVLNPTEFFEDIVIEHEYVECTASAIQALIMFKKLYPGHRKKEIENFVVNAVKYLENTQYPSGGWYGNWGICFIYGTWFALGGLAAGGKTYYNCAAVRKGVDFLLTTQKEDGGWGESYISCPNKEFVPIEGKSNLVQTGWALMGLLHAGQAERDPTPLHRAAKLLINSQLENGDFPQQEITGVFMKNCMLHYPMYRSIYPLWAIAEYRKRVSLPSINST

SEQ ID NO: 50 - protein, 92 aa, Spinacia oleracea
MEFFFLCGLVFYFSISLASFFLFYNHHTTRVYPLPAGEMGWPVVGDSFEFFQTGWNGYPENFIFDRLNKYTPSQVFKTFILREKVVFYLRRL

SEQ ID NO: 51 - DNA, 1563 bp, Spinacia oleracea
  source 1..1563: mol_type = unassigned DNA
atgatagaaatcgggtatattgtaaaatgggtaatttgtttagtgattgttagatgggtatggaagattgtgaattgggtttggtttacaccaaaaaggcttgagaagtttctaagaaaacaaggtttagatggaaattcatacagatttttgttgggtgatctcaaagatatgtctaaaatgcgtaaagaagctagacaaaaacctattccttttactcatgacttctttcatcgtatcttgcctttccacaatcaccatttcaataaatacggggaaagcttcttttcatggatggggcctataccagttgtgaatgttgcagaacaagagcaagtaaagggtgtgttcactaggataaaagagtttcagaaggccaaattaaacccacttgttgcattgcttgtccctggacttgtgagcgctgaaggtgataaatgggtcaagcacaggaagctcatcaacccggcttttcatatggaaaagcttaagcttatgcatccagcatttggcgccagtgttttggatatggtgaacaagtgggagaagatagtatctaaaacaggttcctctgaagtggatgtgtggccgtttgtttccagcctgactgcagatgctatctctcgtgctgcttttggcagtagctatgatgaaggaagaaagatatttgagttggttcttgaacaaactgaaatcaccctacgccttctgcaatcagtttatatccctggatggatgtatgtgccaacaaagaccaacaggaggatgaaaacagtaaactctgaaatacaaaatttattaaccgggataatcgttaagagaaagaaggcaatggaggccggcgaagctgccaaggatgatttgttggggatattgttggagtccaactacaaagatactgaaaatgttctcagtaataagaaaaaactaagcatgactttccaggaattgattgatgagtgcaaactgttctacttagcagggcaagagtcgacctcggtgttgctagcatggacaatgattctgttgggaaagcacacagagtggcaagcacgagcacgagaagaagtagttgcaacgtttggtaaaaacgaacctgattttgaaggcttaaaccatttgaagatagtgacaatgatactgaatgaggtgttgaggttgtaccctccagtgtgtacaatcacccgtaagaatttcaaccacgacgtacagcttggaaatctgacagtccctcgtggtgctatggttacgatgtcagcatatcgtattcaaagagatcctaaaatatggggtgatgatgcaaaagagtttaacccacagagattttcagaaggggttgcaaaggctacaaaggggaatattgcattctttccgtttggttgggggccgcgaatttgcatcggacagaactttgcacttattgaagctaaaatggcagtgtccatggttttacaacgcttttcttttgagctatcaccgtcttatactcatgctcctaccactatcctcactcttcaaccccaacaaggtgctcatctcattatacataagctcagggactaa

SEQ ID NO: 52 - protein, 520 aa, Spinacia oleracea
MIEIGYIVKWVICLVIVRWVWKIVNWVWFTPKRLEKFLRKQGLDGNSYRFLLGDLKDMSKMRKEARQKPIPFTHDFFHRILPFHNHHFNKYGESFFSWMGPIPVVNVAEQEQVKGVFTRIKEFQKAKLNPLVALLVPGLVSAEGDKWVKHRKLINPAFHMEKLKLMHPAFGASVLDMVNKWEKIVSKTGSSEVDVWPFVSSLTADAISRAAFGSSYDEGRKIFELVLEQTEITLRLLQSVYIPGWMYVPTKTNRRMKTVNSEIQNLLTGIIVKRKKAMEAGEAAKDDLLGILLESNYKDTENVLSNKKKLSMTFQELIDECKLFYLAGQESTSVLLAWTMILLGKHTEWQARAREEVVATFGKNEPDFEGLNHLKIVTMILNEVLRLYPPVCTITRKNFNHDVQLGNLTVPRGAMVTMSAYRIQRDPKIWGDDAKEFNPQRFSEGVAKATKGNIAFFPFGWGPRICIGQNFALIEAKMAVSMVLQRFSFELSPSYTHAPTTILTLQPQQGAHLIIHKLRD

SEQ ID NO: 53 - DNA, 1596 bp, Spinacia oleracea
  source 1..1596: mol_type = unassigned DNA
atgatttcaaagagcgcgagtggtgttgcaattgcttcattgggttgcattatcatactgtattggatgtggaaattactaaaagggctatggttaacaccaaaaaagctagagaaatgtctaaaacaacaaggtcttgttggtaattcctacaaatttttgattggagatatgaaagaaagctcaaaattgcgtaacgaagctttacaaaaacccattcctttcactcatgattattacaaccgtattcagcctttcatccatcagattctcaacaattctggtgcaggcaaaaatatctacacatggttgggaccagtgccaacaatactaattacacaacctgagttaataaaggatgctttcaataggatgaacaattttcagaaaccaagattaaatccatatactcaaatgctttcaactggacttccgaactatgagggtcagaaatgggctaaacacaggaagcttctcaaccctgcttttcaacttgataagctcaagcttatgatccatacttttgaaacctgtgttacggatacactgaataagtgggagaagctagtttgtaaaacaggttcttcagaggttgatatatggccatatttgacaactttaacgggagatggtattgctagagctgcatttggaagtagctttgaagatggaagaagaatattcgagcttctcacactgcagaaggatattgttattagtcttctcaaatattcttatattccaggatttaaatatatgccaataaagggtaaccggaagatgaaagaagcggacaatgaaataaaacctctgttgacgaatataattaatagaaggaggaaagcgatggaggccggagaagctcccaaagacgacttgttagggatgctacttgaatccaatgcaaacgaggctcgacaagttaatgaaaatgaaagtggtagtagcaagcgaaaatctgatctaacgatgagcttccctgagatgatcgatgcttgcaagcagttcttcttggctggtcaagagaccacctcagtggccctaacatggacaatgcttttgttagccaagcaccaagattggcaaacacgagctcgacaagaagtacttgctacatttggaatgaataccccagactttgatggcatacataatcgtcttaagattgtgacaatgatactctacgaggtgttaaggttgtatccgccagtccctgcaacatcgcgaagggttcatgatcgtgaaacaaagctaggagatttggtaataccacaaggggtaggagtttcattttccatacttcatgcacacttgaaccctgaaatttggggtgatgatgccaaagaattcaagcctgatagatttgcagaagggattgcaaaagcaacaaaagggaataactcttacttcccctttggttggggacctaggatttgcattggccaaaacttcgcactagttgaggcgaaaatggcattgtgtatgattttgcagcgtttctctttcgatctctcgccttcatacatccatgctccgactagtctcatatcccttcaacctcagcatggtgcccacattattttacatcgattttaa

SEQ ID NO: 54 - protein, 531 aa, Spinacia oleracea
MISKSASGVAIASLGCIIILYWMWKLLKGLWLTPKKLEKCLKQQGLVGNSYKFLIGDMKESSKLRNEALQKPIPFTHDYYNRIQPFIHQILNNSGAGKNIYTWLGPVPTILITQPELIKDAFNRMNNFQKPRLNPYTQMLSTGLPNYEGQKWAKHRKLLNPAFQLDKLKLMIHTFETCVTDTLNKWEKLVCKTGSSEVDIWPYLTTLTGDGIARAAFGSSFEDGRRIFELLTLQKDIVISLLKYSYIPGFKYMPIKGNRKMKEADNEIKPLLTNIINRRRKAMEAGEAPKDDLLGMLLESNANEARQVNENESGSSKRKSDLTMSFPEMIDACKQFFLAGQETTSVALTWTMLLLAKHQDWQTRARQEVLATFGMNTPDFDGIHNRLKIVTMILYEVLRLYPPVPATSRRVHDRETKLGDLVIPQGVGVSFSILHAHLNPEIWGDDAKEFKPDRFAEGIAKATKGNNSYFPFGWGPRICIGQNFALVEAKMALCMILQRFSFDLSPSYIHAPTSLISLQPQHGAHIILHRF

SEQ ID NO: 55 - DNA, 1092 bp, Spinacia oleracea
  source 1..1092: mol_type = unassigned DNA
atgacgggaaaaggaagaacgatggaggtgatcatgatgccatttcaccaccaaggtcacttaaccccgatgctccaattcgcgaagcgcttcgcttggaaaggtgctggctcgatccggatcaccctcgccaccaccctctccaccgcccaaaatatgaccaattccaaaaacaacaacaacaataacgattacgatttcctgacggtcgaaagcatctacgacgataccgatgattctcagctcaaattcatgggtcgtatgggaaagttcaagtccgaagcctcactccaacttggccgtctaatcactactaagagcatcgacaataataaatgtatgctcgtttatgatgcgtatctgccttgggcactggatgtgggcaaggaccataacatacaggctgcggctttcttcgtccaggcttgtgcgtatatggcatccttttaccctatgtttttagaggaatttgggtcggatgatcaacatcctgttgttgcggctgctaaggctgaatctgttcctagtttgtcggttgagctgccgtcgcgggaggaaatggaacgatacgcgccgaaatgtgcacaatccccgagttctgatgataaacccaatactgttaagaaatcgcttcaccctgtctaccggatggtggtttcatcaattacaacccttcatcttgctgatttcgtgctcatcaactcctttgatcaccttgaacatcagctggatgtcttagcacatgaagcagtagggtgtttcataacccattgcggttggaactcgataattgaggcgaccaactttggggttccgatgttggggatgccacagttcatggaccagtttttggatgctcattttatggagaaggtttggggtgttggaattagggctaaggctgatgagaaaaactttgttacttgtgacgaaatcaagtgcggtgtcaatgaaattatgtacggagataaggcaaatatgatcaaggagaatgcagcaaagtggaaagacttggctaaggaggcagttggtgaaggaggcagttcagataagaatatcgacgagatcattaactggcttgcgtcctcctaa

SEQ ID NO: 56 - protein, 363 aa, Spinacia oleracea
MTGKGRTMEVIMMPFHHQGHLTPMLQFAKRFAWKGAGSIRITLATTLSTAQNMTNSKNNNNNNDYDFLTVESIYDDTDDSQLKFMGRMGKFKSEASLQLGRLITTKSIDNNKCMLVYDAYLPWALDVGKDHNIQAAAFFVQACAYMASFYPMFLEEFGSDDQHPVVAAAKAESVPSLSVELPSREEMERYAPKCAQSPSSDDKPNTVKKSLHPVYRMVVSSITTLHLADFVLINSFDHLEHQLDVLAHEAVGCFITHCGWNSIIEATNFGVPMLGMPQFMDQFLDAHFMEKVWGVGIRAKADEKNFVTCDEIKCGVNEIMYGDKANMIKENAAKWKDLAKEAVGEGGSSDKNIDEIINWLASS

SEQ ID NO: 57 - DNA, 1374 bp, Spinacia oleracea
  source 1..1374: mol_type = unassigned DNA
atgggtaaaacagtagcagcttcagagcagttacacatagtaatgatcccatggtttgcttatggacacattcttccctattttgagctttcaaacaaacttgctgaaaaaggccataaaatcaccttggtagttccaaacaaagtcaaacttgatttagaaccaaagatccgtcatccttccttaatcagcttacatgcattcactgtcccacacattgaacccttacctccggggactgagacatgttcagacgtccccattgaacttcagcaccaccttgctgttgccatggacagggcccggcctgaggtggagtccatcatatcagccattgacgatccgaagccggatctgttgttctacgataacgcttactgggtgcctgagatagccacaaagctggggatgaagtctgtgttttaccagattgcatgtgctttaagtatcacccgcattaagcaaaccccgagtgcgagtgcgagtgcgagtgcgagtgccaagctcttcactttacccaagtgggtgctgacgcctaaggtgttaggcgacgcaagagccaattatggagaagggatcacctattaccagagagtgaagaaggctctaagttcctgtgatgctatcgccttacgcacatgccgggagattgaaggggaatcctctgatatcctggctgcacaatataacaagccagtcttcttaacaggtccggtcctacccgaggttgaattcctcccccctttggacaattcctgggctgagtggctagccaagtttgggcctaagtccgtggttttatgttgcttcggaagccagtacgtccctgacaaggctcaactacaggagatggcccttgcccttgaggatactggtcttcccttcttgatgtccgttaagccacccacagagtgcgccaccatagaggaggcgttgccagaagggttctcagagagagtcaaggaacgcggggtggttcatggtggatgggtgcaacagctacaaatactagctcacccatcagtgggttgctttatttgtcattgtgggtacgggtcaatgtgggaggggttgttgagtgataaccagctagtcttattaccacagcttcctgaccagttaatgatggctcaaatgttggcagaaaagctcaaggtgggtgtgatggtggacagagaagaagatgatgggtgggtttccaggaagaacttgtgccaagcagtcaagtctgtcatggatcctcattccgagtttgcagctttactcaagaacaaccatgctaacttcagagacaagttgctaaccaacggttttatggctaattaccttgaagtttttgaccaggatttgaaacgctttcttactgcaaattaa

SEQ ID NO: 58 - protein, 457 aa, Spinacia oleracea
MGKTVAASEQLHIVMIPWFAYGHILPYFELSNKLAEKGHKITLVVPNKVKLDLEPKIRHPSLISLHAFTVPHIEPLPPGTETCSDVPIELQHHLAVAMDRARPEVESIISAIDDPKPDLLFYDNAYWVPEIATKLGMKSVFYQIACALSITRIKQTPSASASASASAKLFTLPKWVLTPKVLGDARANYGEGITYYQRVKKALSSCDAIALRTCREIEGESSDILAAQYNKPVFLTGPVLPEVEFLPPLDNSWAEWLAKFGPKSVVLCCFGSQYVPDKAQLQEMALALEDTGLPFLMSVKPPTECATIEEALPEGFSERVKERGVVHGGWVQQLQILAHPSVGCFICHCGYGSMWEGLLSDNQLVLLPQLPDQLMMAQMLAEKLKVGVMVDREEDDGWVSRKNLCQAVKSVMDPHSEFAALLKNNHANFRDKLLTNGFMANYLEVFDQDLKRFLTAN

SEQ ID NO: 59 - DNA, 1347 bp, Spinacia oleracea
  source 1..1347: mol_type = unassigned DNA
atgggtggagagaaagagttgcggatagtgatgttcccatggcttgcctttggacattttatcccataccttcacctttcaaacaaacttgctgaaaaaggccacaaaatcaccttgttgcttcccaacaaagctaggcttcagttggagtcacttaaccttcatccttctctcataactttccattcaattactgtcccacccctcgaaactctcccttatggcactgaaacaactgcggatatctccctcgaccaacatggtgaactctcgatttccatggaccgcactcggcccgaggtggagtctttcctatcaacccataagcccgacctcgtcctctacgacatggcccattgggtacccgagattgctgctaaggtcgggattaagtcagtttcatacaacgttgtatgtgctattgctgtatctcatgttagacctagcctccctcttccaaaaggaacggcagcacatgtacccctgccattgtcgtctgtccctaagtggagtcttaatcagcacggttcatcaacaccatattttggggaagggataacgttacttgaacggtctgtaatctccctctcgtctgcggatgcaatagccatccgcacgtgcagggagattgaaggggtatattgtgaccgtgttgctgccacattcaacaagcctgtccttgtcaccagccacgccttgcctgatcttgaactcgaactctctccgttggagactcgctgggccgagtggctagctaggttcgagccagggtcagtgatcttttgctgccttggtagtcagcatgtcttagacgcaccccaactgcaagagttggccctggggttggaaatgacaggactacccttcttgatggctgtaaaaccccctgtggggtgtacctccttggaggaggtgcttccagaagggtttaatgatcgggttagcgggcgaggggtggttcacggtgggtgggtgcagcagcagcagataatggcgcacccatcgttagggtgctttgtgaccctttgtgggtcttcgtcgatgtgggaggggttagtgagtgaaagtcagttggtattactcccacaactggcagaccaaactctgtatgccaagttaatggcagatgagctcaaggtgggtgtgaaggtggagagagaagagaacgggtggatgacgaagcgaagtctatgtgaagctatcaagagtgtgatggatgaagatagtgatataagtcatgtagttaggaaaaatcatgctaaatatagaagtatgttgattagccctggctttattagtggctacattgacaacttcatcaaggatttacaagcccttgttccttag

SEQ ID NO: 60 - protein, 448 aa, Spinacia oleracea
MGGEKELRIVMFPWLAFGHFIPYLHLSNKLAEKGHKITLLLPNKARLQLESLNLHPSLITFHSITVPPLETLPYGTETTADISLDQHGELSISMDRTRPEVESFLSTHKPDLVLYDMAHWVPEIAAKVGIKSVSYNVVCAIAVSHVRPSLPLPKGTAAHVPLPLSSVPKWSLNQHGSSTPYFGEGITLLERSVISLSSADAIAIRTCREIEGVYCDRVAATFNKPVLVTSHALPDLELELSPLETRWAEWLARFEPGSVIFCCLGSQHVLDAPQLQELALGLEMTGLPFLMAVKPPVGCTSLEEVLPEGFNDRVSGRGVVHGGWVQQQQIMAHPSLGCFVTLCGSSSMWEGLVSESQLVLLPQLADQTLYAKLMADELKVGVKVEREENGWMTKRSLCEAIKSVMDEDSDISHVVRKNHAKYRSMLISPGFISGYIDNFIKDLQALVP

SEQ ID NO: 61 - DNA, 1476 bp, Spinacia oleracea
  source 1..1476: mol_type = unassigned DNA
atggagctttcaaaccctaccacaacccctaccttaaacgcaacccaacccttacgaggctatttcattccattaatcacagttccaagccacatttccaatcttgttgacattgctaaactcttctcatcacggggagtacatgtgactatcctcaccacccaccacacctccctccgcttcaaacaatccatacatgattggggcttcaaaatcgacctccacatcgtcgacttcccgttcagggaagtcggcttaccggaaggagtggaaaattacagtgatgccacccctgagcaagcaagccagcttttccaggcctttatgatgcttcagaagcctatggaggatgccattcgggctgctaagcccgacttcatcgtttccgataggtattatcattggtctactgatcttgcacgtgagcttgctattccacggctcatctttcatgtcagatgttattttgcattgtgtgctgctgaggttgttgccaagtttgcccctcatgagaaggttgaatctgacactgacctctttttccttcctgacctccctgataccatccacatgacccgtttgcagcttcccgaatggattcagacccgaaacatgttcactgttctcaatgagagaatggacgaggccgatagggagtgctacggtgttattgtaaacagctgctacgagttggagagagcttatgctgacttctaccgcagcaacttgggtcgacgtgcttggtgtatcggtccctatccggtacactgcgacaaggttggtaaaaagaaaggagatgacagcaaaaaacattcatgttttgaatggcttgataaaatgggagaaggagaagttatatacgtgagttttggcactctgtcgtgtttcagccctgctcaaatctcagagctggctactgcactcgaaatgtctggtcacccgtttatctgggtagtaaggaatggtgagaaattgttacctgatggatttgaagaaagaattacagagcaggacaaaggggtgttaataaaagactgggcgccacaagtgaaaatacttgagcacccagctgtaggcggatttctgactcattgtggatggaactcaactgtagaaagtttagcagcaggtgtgccaatggtcacatggccgcttggtgccgagcaattcttcaatgaaaagttgattagtggagttttgaaggtgggggtcgaggtcgggtctgagaagtggagtaggggtatagtaccgaatactgatatgattgagaaggataaaatagaaagggcgattaaagagctgatgagtaaagaacccgaggccgaggaaaggaggcagaaggttaaggagttgagtaaggctccaagaaatgcggttgaagaaggtggttcgtctcgtaacaatttaagtgatttgattgaaaaattacaacgtttaaaggcgaatgaaatatcagtgtccacaaactcagaataa

SEQ ID NO: 62 - protein, 491 aa, Spinacia oleracea
MELSNPTTTPTLNATQPLRGYFIPLITVPSHISNLVDIAKLFSSRGVHVTILTTHHTSLRFKQSIHDWGFKIDLHIVDFPFREVGLPEGVENYSDATPEQASQLFQAFMMLQKPMEDAIRAAKPDFIVSDRYYHWSTDLARELAIPRLIFHVRCYFALCAAEVVAKFAPHEKVESDTDLFFLPDLPDTIHMTRLQLPEWIQTRNMFTVLNERMDEADRECYGVIVNSCYELERAYADFYRSNLGRRAWCIGPYPVHCDKVGKKKGDDSKKHSCFEWLDKMGEGEVIYVSFGTLSCFSPAQISELATALEMSGHPFIWVVRNGEKLLPDGFEERITEQDKGVLIKDWAPQVKILEHPAVGGFLTHCGWNSTVESLAAGVPMVTWPLGAEQFFNEKLISGVLKVGVEVGSEKWSRGIVPNTDMIEKDKIERAIKELMSKEPEAEERRQKVKELSKAPRNAVEEGGSSRNNLSDLIEKLQRLKANEISVSTNSE

SEQ ID NO: 63 - DNA, 1539 bp, Spinacia oleracea
  source 1..1539: mol_type = unassigned DNA
atgggggaagtcaaccatgaagaagtagaaattgaaataatatcaatagaaaccataaaaccatcatcactacttccaccaaaaactcctccaaaaaccatcacactttctcacctcgatcaagctgcccctttgtactactatcctttacttttatactacactaacactactactactaccccaacatcacaaattcgagttgacataacaagtaccctaaaaacttcacttagcaaaacacttgacaaattccaccctattgcaggtcgatgtgtggacgactctacaatttgttgcaaccaccaaggaataccattcattgaaaccaaagttgactccaatatcttggatgtcatgaactcgcctgagaaaatgaagttgcttatcaagtttctccctcatgcagagtttcaagatgtgactcgaccagtctcggatttaaaccatttggcgtttcaagtcaatgttttccggtgtggtggggtgatcattggctcctatgtgctccacaagctccttgatggaatctctcttggaactttctttaaaaattggtcaaccattgctaatgatgagcgagttaaggacgacgacctagtacaacctgactttgaagccactattaaggcgttccctccgcgtacagcaactccaatgcttcctcgtaatcaacaacttccaaaggcggctgaaaaaccaaataataatccagtcaaagttcttgtgacaaagagcttcgtatttgacattgtttctttaaagaagatgatgttcatggctaagagtgaattggttcctaaacccaccaaatttgagaccgtgacagggtttatttgggaacaaaccttatcaacattgcgtaattctggagttgaagttgaacatacatcgcttataatacctgtaaacatccgcccaaggatgagtccgccactcccaagaggatccatgggtaacttgctcaagaatgcaaaggcacaggccaacaccagcagcagcaatgggcttcaagaccttgttaaagaaatccattcatctttgtctcaaacaacccagaaaattaatactcctcctcctcctcctcctcctcctcctactactactgctacaacaatccattcatctttgtctcaaacaacccagaaaattaatactcctcctcctactactacaacaatccattcatctttgtctcaaacaacccagaaaattaatactactactactacagcagaggttattttgactaaacggaaagttgacaatccagttacacagaatcgagaaggaaactacctcttcaccagttggtgcaagattgggttggatgaggctgacttcgggttcggaaagcccgtttgggtaattcccaacgatgggagaccccctaaggtcaggaatatgattttccttactgattataggcatcccgaaacaggcgttgaaggaattgcagcatggattacgttggaagagaaacaaatgcaatgtttaaagtcaaacccagaattccttgcttttgctactcctaattag

SEQ ID NO: 64 - protein, 512 aa, Spinacia oleracea
MGEVNHEEVEIEIISIETIKPSSLLPPKTPPKTITLSHLDQAAPLYYYPLLLYYTNTTTTTPTSQIRVDITSTLKTSLSKTLDKFHPIAGRCVDDSTICCNHQGIPFIETKVDSNILDVMNSPEKMKLLIKFLPHAEFQDVTRPVSDLNHLAFQVNVFRCGGVIIGSYVLHKLLDGISLGTFFKNWSTIANDERVKDDDLVQPDFEATIKAFPPRTATPMLPRNQQLPKAAEKPNNNPVKVLVTKSFVFDIVSLKKMMFMAKSELVPKPTKFETVTGFIWEQTLSTLRNSGVEVEHTSLIIPVNIRPRMSPPLPRGSMGNLLKNAKAQANTSSSNGLQDLVKEIHSSLSQTTQKINTPPPPPPPPPTTTATTIHSSLSQTTQKINTPPPTTTTIHSSLSQTTQKINTTTTTAEVILTKRKVDNPVTQNREGNYLFTSWCKIGLDEADFGFGKPVWVIPNDGRPPKVRNMIFLTDYRHPETGVEGIAAWITLEEKQMQCLKSNPEFLAFATPN

SEQ ID NO: 65 - DNA, 2091 bp, Spinacia oleracea
  source 1..2091: mol_type = unassigned DNA
atggcaacttctcacattcgcaatgtccaattaaccagagccattgttaaccgtctccacatcttcctccattccgtagccatcttctcgctcttctactaccgtttcacttccttcttcaactccgacatctccatacttgcttactccttactcaccaccgccgaactcttcttaacctttctatgggcttttactcaggctttccggtggcgtcccgtaatgagggaagtctccgggtacgaatccatcaaacccgaacaactaccgggtttggatgtcttcattgtcactgctgacccgacaaaggagccagttctggaggtgatgaactccgtgatatcatccatggctttggattatccggttgatagactggcggtttacttgtcggatgacggtggttctccgttgtcgaaggaggcgattaagaaggcttatgagtttgctaagctttggattcctttttgtaataagtataatgttaagacaaggtgtcctcaggctttcttctcgcctcttgctgatggggaaaggcttgattggaattctgagtttatggctgatcaattggaactccagaccaaatatgaagcttttagaaactatgtggagaaagaaagtggagataacaccaaatgtactgcagttcatgatcgacctccttgcgttgagattatacatgacaacaaacagaacggagaaagtgatgtgaagatgccccttctggtttatgtagccagggaaaagagacctggtcgtcctcatcgtttcaaagctggagcccttaatgctcttcttcgagtatccagtttaatgagcaatgcaccttacttattggtgttggattgtgatatgtactgccatgatccaacttctgctcgtcaatctatgtgcttccatcttgacacaaacatggcttcctctcttgcatatgtgcaataccctcaaattttctataatgttagcaaaaatgacatctatgatggccaagccagatcagctcatatgacgaaatggaaaggcatggatggactcagaggcccggtcttgaatggaactgggtattatttgaagcgaaaagcattatttggaaagcctaataacgaagatgaatacctcaacagtcaaccagaaaaggcctttggctcctccacaaaattaattgctgcactaagagagaactccaagcaaaatcttgccataaaggaattgacagaagatgagttgtaccaagaggctagaaatttggctacttgcacatatgaagcaaacacactatggggcagtgaggtaggatattcgtatgagtgcttgttggagagtacattcactggatatatgttacattgcagaggatggaaatctgtgtatctttacccaaaaagaccatgcttcttgggatgcacaacgattgatatgaaggatgctacggttcaactaataaaatggacctcctcattacttggaattgccctgtcgaagtctagccctctaactttggccatgtccagtatgtcaatcctgcaaagcatgtgttacgcgtacatcacatttacaggcctttttgcagctccattggttatatatggtgttgtccttccaataagcctattgaagggctttcctattttccctaaggtatcggatccatggattttgccatttgtgttgatatttgtatcctcccatcttcaacatctatatgaggtcctggaaagtgacaaatcagcaacacaatggtggaatgaggtgagaatttggatgatgaaatcagtgacagcctgtttgtttgggttgacggaagcgataatgaagaagattggagtacaaactgcaacattcagattaacaaataaggtagttgagaaggaaaagatggataaatacgagaaggagaggtttgatttctcaggagcagctatgcttatggttcctcttaatattttggtggtactaaatatggtgtcattcattggtggactcatgagggtcataatcaacaacagttatgatcaaatgtttgcacaacttttcctctccttttttgtcctacttcttagctaccctgttgttaagggatggttataa

SEQ ID NO: 66 - protein, 696 aa, Spinacia oleracea
MATSHIRNVQLTRAIVNRLHIFLHSVAIFSLFYYRFTSFFNSDISILAYSLLTTAELFLTFLWAFTQAFRWRPVMREVSGYESIKPEQLPGLDVFIVTADPTKEPVLEVMNSVISSMALDYPVDRLAVYLSDDGGSPLSKEAIKKAYEFAKLWIPFCNKYNVKTRCPQAFFSPLADGERLDWNSEFMADQLELQTKYEAFRNYVEKESGDNTKCTAVHDRPPCVEIIHDNKQNGESDVKMPLLVYVAREKRPGRPHRFKAGALNALLRVSSLMSNAPYLLVLDCDMYCHDPTSARQSMCFHLDTNMASSLAYVQYPQIFYNVSKNDIYDGQARSAHMTKWKGMDGLRGPVLNGTGYYLKRKALFGKPNNEDEYLNSQPEKAFGSSTKLIAALRENSKQNLAIKELTEDELYQEARNLATCTYEANTLWGSEVGYSYECLLESTFTGYMLHCRGWKSVYLYPKRPCFLGCTTIDMKDATVQLIKWTSSLLGIALSKSSPLTLAMSSMSILQSMCYAYITFTGLFAAPLVIYGVVLPISLLKGFPIFPKVSDPWILPFVLIFVSSHLQHLYEVLESDKSATQWWNEVRIWMMKSVTACLFGLTEAIMKKIGVQTATFRLTNKVVEKEKMDKYEKERFDFSGAAMLMVPLNILVVLNMVSFIGGLMRVIINNSYDQMFAQLFLSFFVLLLSYPVVKGWL

SEQ ID NO: 67 - protein, 1081 aa, Arabidopsis thaliana
MEASAGLVAGSYRRNELVRIRHESDGGTKPLKNMNGQICQICGDDVGLAETGDVFVACNECAFPVCRPCYEYERKDGTQCCPQCKTRFRRHRGSPRVEGDEDEDDVDDIENEFNYAQGANKARHQRHGEEFSSSSRHESQPIPLLTHGHTVSGEIRTPDTQSVRTTSGPLGPSDRNAISSPYIDPRQPVPVRIVDPSKDLNSYGLGNVDWKERVEGWKLKQEKNMLQMTGKYHEGKGGEIEGTGSNGEELQMADDTRLPMSRVVPIPSSRLTPYRVVIILRLIILCFFLQYRTTHPVKNAYPLWLTSVICEIWFAFSWLLDQFPKWYPINRETYLDRLAIRYDRDGEPSQLVPVDVFVSTVDPLKEPPLVTANTVLSILSVDYPVDKVACYVSDDGSAMLTFESLSETAEFAKKWVPFCKKFNIEPRAPEFYFAQKIDYLKDKIQPSFVKERRAMKREYEEFKVRINALVAKAQKIPEEGWTMQDGTPWPGNNTRDHPGMIQVFLGHSGGLDTDGNELPRLIYVSREKRPGFQHHKKAGAMNALIRVSAVLTNGAYLLNVDCDHYFNNSKAIKEAMCFMMDPAIGKKCCYVQFPQRFDGIDLHDRYANRNIVFFDINMKGLDGIQGPVYVGTGCCFNRQALYGYDPVLTEEDLEPNIIVKSCCGSRKKGKSSKKYNYEKRRGINRSDSNAPLFNMEDIDEGFEGYDDERSILMSQRSVEKRFGQSPVFIAATFMEQGGIPPTTNPATLLKEAIHVISCGYEDKTEWGKEIGWIYGSVTEDILTGFKMHARGWISIYCNPPRPAFKGSAPINLSDRLNQVLRWALGSIEILLSRHCPIWYGYHGRLRLLERIAYINTIVYPITSIPLIAYCILPAFCLITDRFIIPEISNYASIWFILLFISIAVTGILELRWSGVSIEDWWRNEQFWVIGGTSAHLFAVFQGLLKVLAGIDTNFTVTSKATDEDGDFAELYIFKWTALLIPPTTVLLVNLIGIVAGVSYAVNSGYQSWGPLFGKLFFALWVIAHLYPFLKGLLGRQNRTPTIVIVWSVLLASIFSLLWVRINPFVDANPNANNFNGKGGVF

SEQ ID NO: 68 - protein, 1065 aa, Arabidopsis thaliana
MESEGETAGKPMKNIVPQTCQICSDNVGKTVDGDRFVACDICSFPVCRPCYEYERKDGNQSCPQCKTRYKRLKGSPAIPGDKDEDGLADEGTVEFNYPQKEKISERMLGWHLTRGKGEEMGEPQYDKEVSHNHLPRLTSRQDTSGEFSAASPERLSVSSTIAGGKRLPYSSDVNQSPNRRIVDPVGLGNVAWKERVDGWKMKQEKNTGPVSTQAASERGGVDIDASTDILADEALLNDEARQPLSRKVSIPSSRINPYRMVIMLRLVILCLFLHYRITNPVPNAFALWLVSVICEIWFALSWILDQFPKWFPVNRETYLDRLALRYDREGEPSQLAAVDIFVSTVDPLKEPPLVTANTVLSILAVDYPVDKVSCYVFDDGAAMLSFESLAETSEFARKWVPFCKKYSIEPRAPEWYFAAKIDYLKDKVQTSFVKDRRAMKREYEEFKIRINALVSKALKCPEEGWVMQDGTPWPGNNTGDHPGMIQVFLGQNGGLDAEGNELPRLVYVSREKRPGFQHHKKAGAMNALVRVSAVLTNGPFILNLDCDHYINNSKALREAMCFLMDPNLGKQVCYVQFPQRFDGIDKNDRYANRNTVFFDINLRGLDGIQGPVYVGTGCVFNRTALYGYEPPIKVKHKKPSLLSKLCGGSRKKNSKAKKESDKKKSGRHTDSTVPVFNLDDIEEGVEGAGFDDEKALLMSQMSLEKRFGQSAVFVASTLMENGGVPPSATPENLLKEAIHVISCGYEDKSDWGMEIGWIYGSVTEDILTGFKMHARGWRSIYCMPKLPAFKGSAPINLSDRLNQVLRWALGSVEILFSRHCPIWYGYNGRLKFLERFAYVNTTIYPITSIPLLMYCTLLAVCLFTNQFIIPQISNIASIWFLSLFLSIFATGILEMRWSGVGIDEWWRNEQFWVIGGVSAHLFAVFQGILKVLAGIDTNFTVTSKASDEDGDFAELYLFKWTTLLIPPTTLLIVNLVGVVAGVSYAINSGYQSWGPLFGKLFFAFWVIVHLYPFLKGLMGRQNRTPTIVVVWSVLLASIFSLLWVRIDPFTSRVTGPDILECGINC

SEQ ID NO: 69 - protein, 760 aa, Arabidopsis thaliana
METHRKNSVVGNILHTCHPCRRTIPYRIYAIFHTCGIIALMYHHVHSLVTANNTLITCLLLLSDIVLAFMWATTTSLRLNPVHRTECPEKYAAKPEDFPKLDVFICTADPYKEPPMMVVNTALSVMAYEYPSDKISVYVSDDGGSSLTFFALIEAAKFSKQWLPFCKKNNVQDRSPEVYFSSESHSRSDEAENLKMMYEDMKSRVEHVVESGKVETAFITCDQFRGVFDLWTDKFSRHDHPTIIQVLQNSETDMDNTRKYIMPNLIYVSREKSKVSPHHFKAGALNTLLRVSGVMTNSPIILTLDCDMYSNDPATLVRALCYLTDPEIKSGLGYVQFPQKFLGISKNDIYACENKRLFIINMVGFDGLMGPTHVGTGCFFNRRAFYGPPYMLILPEINELKPYRIADKSIKAQDVLSLAHNVAGCIYEYNTNWGSKIGFRYGSLVEDYYTGFMLHCEGWRSVFCNPKKAAFYGDSPKCLVDLVGQQIRWAVGLFEMSFSKYSPITYGIKSLDLLMGLGYCNSPFKPFWSIPLTVYGLLPQLALISGVSVFPKASDPWFWLYIILFFGAYAQDLSDFLLEGGTYRKWWNDQRMLMIKGLSSFFFGFIEFILKTLNLSTPKFNVTSKANDDDEQRKRYEQEIFDFGTSSSMFLPLTTVAIVNLLAFVWGLYGILFCGGELYLELMLVSFAVVNCLPIYGAMVLRKDDGKLSKRTCFLAGNLHVGSYCVKLLRPQVTSPLRLIHNNNTSGWFKRKKHNMNESV

SEQ ID NO: 70 - protein, 1086 aa, Spinacia oleracea
MEATGGMVAGSYKRNELVRIRHDSTDSGSKSLKNLDGQICQICGDTVGVTSNGGVFVACNECAFPVCRPCYEYERKDGNQCCPQCKTRYKRQKGSLRVEGDDEEEDVDDLDNEFNYERGTSKARHQWQGEDVDLSSSSRHGSQPIPLLTNGQVVSGEIPSATPDNQSVRSTSGPIGPEKRGNHSLPYIDPCLPVPVRIVDPSKDLNSYGLGSVDWKERVESWKLKQEKNMTHTGNRYSEGKGGDVEGSGSNGEELQLADDVRQPMSRIVPIPSSHLTPYRAVIIFRLIILVFFLQFRITHPVEDAYPLWLTSVICEIWFAMSWILDQFPKWYPINRETYLDRLAFRHDREGEPSQLAPIDVFVSTVDPLKEPPIITANTVLSILAVDYPVDKVSCYVSDDGSAMLTFEGLSETAEFARKWVPFCKKFSIEPRAPEFYFQQKIDYLKDKIQPSFVKERRAMKREYEEFKVRINALVAKAQKVPEEGWTMQDGTAWPGNNPRDHPGMIQVFLGHSGGLDMDGNELPRLVYVSREKRPGFQHHKKAGAMNALIRVSAVLTNGAYILNVDCDHYFNNSKCLKEAMCFMMDPALGKKVCYVQFPQRFDGIDLHDRYANRNIVFFDINMKGQDGIQGPVYVGTGCCFNRQALYGYDPVLTEEDFEPNFIIKNCFGSRKKGKSGNKKYMDKKRGPKRSESSIPIFNMEDIEEGVEGYEDEKSLLMSQKRLEKRFGQSPVFIAATFMEMGGIPPTTNPATLLKEAIHVISCGYEDKSEWGKEIGWIYGSVTEDILTGFKMHARGWMSIYCMPPRPAFKGSAPLNLSDRLNQVLRWALGSIEIMLSRHCPIWYGYKGRLRFLERLAYINTVVYPLTSIPLIAYCILPAICLLTNKFIIPTLSNFASILFIMLFMSIAATGILELRWSGVSIEDWWRNEQFWVIGGTSAHLFAVFQGLLKVLAGIDTNFTVTSKAADEDGDFAELYIFKWTALLIPPTTVLIVNLVGVVAGVSYAINSGYQSWGPLFGKLFFSFWVIAHLYPFLKGLLGRQNRTPTIVIVWSVLLASIFSLLWVRINPFTTDAEKAAAGNQCGINC

SEQ ID NO: 71 - protein, 1060 aa, Spinacia oleracea
MMEDSQSGVKPTKQANEQVCQICSDNIGTTVDGEPFVACDVCSFPVCRACYEYERKDGTQSCPQCKTRYKRQKGSPAIHGEKVEDSDVEDVVSDVNEPLGSSILKEKPQERMLGWHMNHGQSGELGPPTYDKEAPISHIPRLATGRTVSGDLSAASPGRFSMPSPGASTGANIRVSREFASPGFGNVAWKERIDGWKMKQEKSTGPPSVSHAPSEGRFANDIDASTEIAMDDPLLNDETRQPLSRKVPIPSSRINPYRMVIVLRLAVLGIFLHYRVTNPVPNAYALWLISVICEIWFAFSWILDQFPKWLPINRETYLDRLALRYDREGEPSQLAAVDIFVSTVDPLKEPPLVTANTVLSILAVDYPVDKVSCYVSDDGAAMLTFEALSETSEFARKWVPFTKKYNIEPRAPEWYFSQKIDYLKDKVQTTFVKDRRAMKREYEEFKIRINGLVAKATKVPEEGWVMQDGTPWPGNNTRDHPGMIQVFLGQSGGLDTDGNELPRLVYVSREKRPGFTHHKKAGAMNSLVRVSAVLTNGPFMLNLDCDHYINNSKALREAMCFMMDPNLGKYCCYVQFPQRFDGIDRNDRYANRNTVFFDINLRGLDGIQGPVYVGTGCVFNRTALYGYEPPIKPKPKKKGILSSCFGGSRKKSSKKDSKKKSKHADPTVPIFNLEDIEEGVEGTGFDDEKSLLMSQISLEKRFGKSEVFVASTLMENGGVPQSATPDTLLKEAIHVISCGYEDKTDWGAEIGWIYGSVTEDILTGFKMHARGWRSIYCMPKLAAFKGSAPINLSDRLNQVLRWALGSVEILFSRHCPLWYGYGGRLKWLERFAYINTTIYPLTSIPLLAYCTLPAVCLLTGKFIIPQISNLASVWFLSLFLSIFATGILEMRWSGVGIDEWWRNEQFWVIGGISAHLFAVFQGLLKVLAGIDTNFTVTSKASDEDGDFTELYLFKWTTLLIPPTTILIVNLVAVVAGISYAINSGYQSWGPLFGKLFFAFWVIVHLYPFLKGLMGRQNRTPTIVVVWSILLASIFSLLWVRVDPFTTRVTGPDVHICGINC

SEQ ID NO: 72 - DNA, 1431 bp, Spinacia oleracea
  source 1..1431: mol_type = unassigned DNA
atgaaagaaagctctactttgcgtgctcaagctttgaacaaacccattccttttactcatgattactaccaacgtattcagcctttcacccatcagattctcaacaattcttgtgcaggtgcaggtaaaaatatctacacatggttgggaccagtgccaacaatactaattacacagcctgagttaataaaggatgctttcaataggatgaacaatttccaaaaaccaagattgaatccatatactcaaatactttcaactggacttccgaactatgagggccagaaatgggctaaacacaggaagcttctcaaccctgcttttcaacttgataagctcaagcttatgatccctgcttttgaaacctgcgttactgatacactaaataagtgggagaagctagtttctaaaacaggttcttcagaggttgatgtgtggccacatttcacaactctaacgggagatggtattgctagagctgcatttggaagtagctttgaggatggaagaaggatattcgaccttcttaaagagcaaaaggatcttgttattagtcttctcaaatattcttatattccaggattcaaatatttgcccacaaaaggtaacaagatgatgaaagaaacggaaaacgaaatcaaacctttattgacgaatttgattcacaaaaggaagaaggcaatggaggctggagaagctcccaaagacgacttgttgggaatgctacttgaatccaatgcaaacgaggctcgacaagttaacgaaaatgaaagtggaagtaggaagcgacaatccgatataacaatgagcttccatgagatgattgacgcgtgcaagttgttcttcttggctggccaagagactacttctgtggcgctaacatgggcaatgattttgttggcgaagcaccaagattaccaaacacgagcacgagaagaagtacttgctacatttggaacgaaaactcctgactttgatggtgtacataatcgccttaagattgtgacaactatagtccacgaggtgctaagattgtatccgccaatccctgcaacatcacgaagggcacatgaacgcgaaacaaaactaggagatttggtaataccacaaggggtaggagtttcattttccatactacatgctcacttgaaccctgaaatttggggtgatgatgccaaagaattcaagcctgaaagattttcagaagggattgcaaaggcaaccaaaggaaataactcttacttcccctttggttggggacctaggatttgcattggtcaagccttcgcactaatccaggttaaaatggcattgtctatgattttacaacgtttctctttcgagctttcaccgtcctacatccatgctccaactagtctattagcccttcaaccccagcatggtgctcatgttatcttacatcgactttaa

SEQ ID NO: 73 - protein, 476 aa, Spinacia oleracea
MKESSTLRAQALNKPIPFTHDYYQRIQPFTHQILNNSCAGAGKNIYTWLGPVPTILITQPELIKDAFNRMNNFQKPRLNPYTQILSTGLPNYEGQKWAKHRKLLNPAFQLDKLKLMIPAFETCVTDTLNKWEKLVSKTGSSEVDVWPHFTTLTGDGIARAAFGSSFEDGRRIFDLLKEQKDLVISLLKYSYIPGFKYLPTKGNKMMKETENEIKPLLTNLIHKRKKAMEAGEAPKDDLLGMLLESNANEARQVNENESGSRKRQSDITMSFHEMIDACKLFFLAGQETTSVALTWAMILLAKHQDYQTRAREEVLATFGTKTPDFDGVHNRLKIVTTIVHEVLRLYPPIPATSRRAHERETKLGDLVIPQGVGVSFSILHAHLNPEIWGDDAKEFKPERFSEGIAKATKGNNSYFPFGWGPRICIGQAFALIQVKMALSMILQRFSFELSPSYIHAPTSLLALQPQHGAHVILHRL

SEQ ID NO: 74 - DNA, 1443 bp, Spinacia oleracea
  source 1..1443: mol_type = unassigned DNA
atggtgaagatttgctgcattggggctggttatgtaggaggccctactatggctgttatagcactcaagtgcccaaagattgaagttgtagtggttgatatatctgtgtctcggatcactgcatggaacagcgagcagcttccaatctatgagccgggtctagatgatgtggttaaggaatgccgtggaaggaaccttttcttcagcactgatgtagaaaagcatgttgctgaggctgatattgtttttgtctctgtgaatacccctaccaaaaccacaggtcttggagcaggcaaagctgctgatttgacctactgggagagtgctgcccgtatgattgctgatgtttcaaagtctgacaaaatcgttgttgagaaatcaactgtgccagtgaaaactgctgaggcaatcgaaaagattctgacgcacaacagcaagggaatcaactaccagatcctttcaaatccggagttccttgctgaaggtactgctattcaggacctttttcaccctgacagggttctcatcggtggccgggaaaccccagcaggcctcaaggcagtccaagcattgaaggatgtgtatgctcaatgggttcctgatgaacggatcttaaccaccaatctttggtctgctgagctctcaaagcttgctgccaacgccttcttagcacagaggatttcatctgtcaatgcaatgtcagctctttgtgaggctactggagcagatgttacccaagtcgcatatgctgttggtaaggacagtaggattgggcaaaagtttttgaacgctagtgttggttttggagggtcttgcttccagaaagacattctgaacttggtttacatttgtgagtgcaacggtctccctgaggtggccgagtattggaaacaggtaatcaaggtgaatgattatcagaagaatcgttttgtgaatagggttgtggcctccatgttcaacactgtatcgaacaagaagattgctgttcttggatttgcattcaaaaaggatacaggggatactagggagacacccgccatagatgtgtgcaagggtttgttgggagacaaggcaaggttgagcatctacgatccacaagtcactgaggatcagattcagcgagatctcaccatgaacaagtttgactgggaccacccaattcacctccagcccacaagtcccacaactgttaagcaagtgagtgttgtttgggacgcttatgaggccaccaaagatgctcatgctgtgtgtattctgactgagtgggatgaatttaagaaacttgattacaaaaggatttttgacaacatgcagaagccagcttttgtgtttgatggaaggaacattgtgaatgcagatgagctgaggcagattgggttcattgtgtactcaattggtaaacctttggattcatggctcaaggacatgcctgctgtggcttaa

SEQ ID NO: 75 - protein, 480 aa, Spinacia oleracea
MVKICCIGAGYVGGPTMAVIALKCPKIEVVVVDISVSRITAWNSEQLPIYEPGLDDVVKECRGRNLFFSTDVEKHVAEADIVFVSVNTPTKTTGLGAGKAADLTYWESAARMIADVSKSDKIVVEKSTVPVKTAEAIEKILTHNSKGINYQILSNPEFLAEGTAIQDLFHPDRVLIGGRETPAGLKAVQALKDVYAQWVPDERILTTNLWSAELSKLAANAFLAQRISSVNAMSALCEATGADVTQVAYAVGKDSRIGQKFLNASVGFGGSCFQKDILNLVYICECNGLPEVAEYWKQVIKVNDYQKNRFVNRVVASMFNTVSNKKIAVLGFAFKKDTGDTRETPAIDVCKGLLGDKARLSIYDPQVTEDQIQRDLTMNKFDWDHPIHLQPTSPTTVKQVSVVWDAYEATKDAHAVCILTEWDEFKKLDYKRIFDNMQKPAFVFDGRNIVNADELRQIGFIVYSIGKPLDSWLKDMPAVA

SEQ ID NO: 76 - DNA, 1482 bp, Glycyrrhiza uralensis
  source 1..1482: mol_type = unassigned DNA
atggaagtacattgggtttgcatgtccgctgccactttgttggtatgctacatttttggaagcaagtttgtgaggaatttgaatgggtggtattatgatgtaaaactaagaaggaaagaacacccactacccccaggtgacatgggatggcctcttatcggcgatctattgtccttcatcaaagatttctcatcgggtcaccctgattcattcatcaacaaccttgttctcaaatatggacgaagtggtatctacaagactcacttgtttgggaatccaagcatcattgtttgygagcctcagatgtgtaggcgagttctcactgatgatgtgaactttaagcttggttatccaaaatctatcaaagagttggcacgatgtagacccatgattgatgtctctaatgcggaacataggctttttcgacgcctcattacttccccaatcgtgggtcacaaggcgctagcaatgtacctagagcgtcttgaggaaattgtgatcaattcgttggaagaattgtccagcatgaagcaccccgttgagctcttgaaagagatgaagaaggtttcctttaaagccattgtccacgtyttcatgggctcttccaatcaggacatcattaaaaaaattggaagttcgtttactgatttgtacaatggcatgttctctatccccattaacgtacctggttttacattccacaaagcactcgaggcacgtaagaagctagccaaaatagttcaacccgttgtggatgaaaggcggttgatgatagaaaatggtccacaagaagggagccaaagaaaagatcttattgatattcttttggaagtcaaagatgagaatggacgaaaattggaggacgaggatattagcgatttattaatagggcttttgtttgctggccatgaaagtacagcaaccagtttaatgtggtcaattacatatcttacacagcatccccatatcttgaaaaaggctaaggaagagcaggaagaaataacgaggacaagattttcctcgcagaaacaattaagtcttaaggaaattaagcaaatggtttatctttctcaggtaattgatgaaactttacgatgtgccaatattgcctttgcaacttttcgagaggcaactgctgatgtgaacatcaatggttatatcataccaaagggatggagagtgctaatttgggcaagagccattcatatggattctgaatattacccaaatccagaagaatttaatccatcgagatgggatgattacaatgccaaagcaggaaccttccttccttttggagcaggaagtagactttgtcctggagccgacttggcgaaacttgaaatttccatatttcttcattatttcctccttaattacaggttggagagaataaatccagaatgtcacgttaccagcttaccagtatctaarcccacagacaattgtctcgctaaggtgataaaggtctcatgtgcttag

SEQ ID NO: 77 - protein, 493 aa, Glycyrrhiza uralensis
  VARIANT 98: note = misc_feature - Xaa can be any naturally occurring amino acid
  VARIANT 478: note = misc_feature - Xaa can be any naturally occurring amino acid
MEVHWVCMSAATLLVCYIFGSKFVRNLNGWYYDVKLRRKEHPLPPGDMGWPLIGDLLSFIKDFSSGHPDSFINNLVLKYGRSGIYKTHLFGNPSIIVXEPQMCRRVLTDDVNFKLGYPKSIKELARCRPMIDVSNAEHRLFRRLITSPIVGHKALAMYLERLEEIVINSLEELSSMKHPVELLKEMKKVSFKAIVHVFMGSSNQDIIKKIGSSFTDLYNGMFSIPINVPGFTFHKALEARKKLAKIVQPVVDERRLMIENGPQEGSQRKDLIDILLEVKDENGRKLEDEDISDLLIGLLFAGHESTATSLMWSITYLTQHPHILKKAKEEQEEITRTRFSSQKQLSLKEIKQMVYLSQVIDETLRCANIAFATFREATADVNINGYIIPKGWRVLIWARAIHMDSEYYPNPEEFNPSRWDDYNAKAGTFLPFGAGSRLCPGADLAKLEISIFLHYFLLNYRLERINPECHVTSLPVSXPTDNCLAKVIKVSCA

SEQ ID NO: 78 - DNA, 1617 bp, Glycyrrhiza uralensis
  misc_difference 1009: note = misc_feature - n is a, c, g, or t
  source 1..1617: mol_type = unassigned DNA
atggatgcatcttccacaccaggggctatctgggttgttctgacagtgatactagctgcgattcccatatgggcatgccatatggtgaacacgctgtggctgaggccaaagaggttggaaaggcatctcagagctcaaggtcttcatggtgacccttacaagctctcacttgacaactccaagcaaacctatatgctcaagttgcaacaagaagcacaatcaaaatccattggtctctccaaagatgatgctgcaccacgaatcttctcccttgcccatcaaactgtacacaaatatggaaagaactcctttgcatgggaagggacagcaccaaaggtgatcatcacagacccagagcaaattaaggaagtctttaacaagattcaggacttccccaaaccaaaattaaatcccatcgccaagtatattagcatcggtctaatacagtatgagggtgacaaatgggccaaacatcgaaagattatcaatccggcattccacttagaaaaattgaaaggtatgctgccagcattttctcatagctgccatgaaatgattagcaaatggaaggggttattgtcatcagatggaacatgtgaggttgatgtttggcccttccttcaaaatctcacttgtgatgtaatttctaggacggcattcggaagcagctatgcagaaggagcaaaaatatttgaacttttgaaaaggcagggatatgctttgatgacagcacgatacgcacgcattccattatggtggcttctaccatcaactaccaaaaggaggatgaaggaaattgaaagaggcatacgtgattcacttgaaggtatcattagaaaacgagaaaaagcattgaagagtggcaaaagcaccgatgacgacttattaggcatacttttgcaatcaaatcacattgaaaataaaggagatgaaaacagtaagagtgctggaatgaccacccaagaagtaatggaggaatgcaaacttttttacctggcagggcaagagnttgaaaataaaggagatgaaaacagtaagagtgctggaatgaccacccaagaagtaatggaggaatgcaaacttttttacctggcagggcaagagaccaccgcagctttgctggcctggacaatggtgttattaggcaagcatcctgaatggcaagcacgcgcaaggcaggaagttttgcaagtaaccatgattttatatgaggtactcaggctgtacccacctgggatttacctcacccgagctcttcgaaaggatttgaaacttggaaaccttttgctacctgctggagtacaggtttccgtaccaatacttttgattcaccatgatgaaggtatatggggcaatgatgcaaaggagttcaatcctgaaaggtttgctgaaggaattgcaaaggcaacaaaaggccaagtttgctatttcccttttggatggggtcctagaatatgtgttgggcaaaactttgccttattagaagccaagattgtattgtcattgctgctgcagaatttctcatttgagctatctccgacttatgcacatgttcctaccacggtgcttactttgcagccaaaacatggggcacccatcattctgcataaactgtaa

SEQ ID NO: 79 - protein, 538 aa, Glycyrrhiza uralensis
  VARIANT 337: note = misc_feature - Xaa can be any naturally occurring amino acid
MDASSTPGAIWVVLTVILAAIPIWACHMVNTLWLRPKRLERHLRAQGLHGDPYKLSLDNSKQTYMLKLQQEAQSKSIGLSKDDAAPRIFSLAHQTVHKYGKNSFAWEGTAPKVIITDPEQIKEVFNKIQDFPKPKLNPIAKYISIGLIQYEGDKWAKHRKIINPAFHLEKLKGMLPAFSHSCHEMISKWKGLLSSDGTCEVDVWPFLQNLTCDVISRTAFGSSYAEGAKIFELLKRQGYALMTARYARIPLWWLLPSTTKRRMKEIERGIRDSLEGIIRKREKALKSGKSTDDDLLGILLQSNHIENKGDENSKSAGMTTQEVMEECKLFYLAGQEXENKGDENSKSAGMTTQEVMEECKLFYLAGQETTAALLAWTMVLLGKHPEWQARARQEVLQVTMILYEVLRLYPPGIYLTRALRKDLKLGNLLLPAGVQVSVPILLIHHDEGIWGNDAKEFNPERFAEGIAKATKGQVCYFPFGWGPRICVGQNFALLEAKIVLSLLLQNFSFELSPTYAHVPTTVLTLQPKHGAPIILHKL

SEQ ID NO: 80 - DNA, 2088 bp, Glycyrrhiza uralensis
  source 1..2088: mol_type = unassigned DNA
atggcaagcttcacccttcacacagaaaccgttcagtcatggctactcctcagcagacttcacatactgctgcacctcgcagttgtactgctcctcttatactaccgcatcacacgtttccccttccatgctccsactctaccgtggactctgatgaccgtaggtgaggctattatggcagtgctgtggttcttcaaccaggccttccggtggcggccggtgagccgctcggtgatgacggagaagctgcccagcgacgcgaagctgccggggcttgacatattcgtgtgcacgcttgaccccgagaaggagcccaccgtggaggtgatgaacactctggtctctgcccttgccatggactacccccctgacaagctctccgtttacctctccgacgatggcgccgccccggtcactctttacggcgtgagagaggcttctgagttcgcgagggtgtgggtccctttctgcaaaaagtatgggatcaagtcaaggtgtcccaaggttttcttctctcccagtgctgaggatgaacaccttcttcgcaccgacgagttcaggtcagagcgagacctcatcaaggctaaatacgagaaaatgcagaaaaatattgagaaatttggttcggatgccaaaaattgtcgtatggtgactgacagacctcctcggatcgagatattgattgaccaaccagacatgccacgtgttgtttacgtgtctcgggaaagaaggccatcactccctcacaagttcaaaggaggagccctcaatacattgctcagagtctcaggtctaatcagcaatgggccttatgtacttgtagtggactgtgatatgtattgcaatgacccatcctcagccaaacaagccatgtgtttctttcttgatcctgaaacctctaaatytattgcatttgtccaattccctcaaatgtttcacaaccttggcaaaaaagacatctatgacaatcaatctaggactgcttttaagacaatgtggcaagggatggatggactaagaggtcctggtctttctggcagcggtaattacttgaatagaagtgcattactatttggaagtccaaatcaaaaagatgactatctggatgatgcccaaaactacttrggcaagtctaccatgtacatagaatcactaaaggccattcgtggacaaaaaactatgaaaaagaatatttcaagagatgaaattttacgagaagctcaagtattagcctcttgttcctatgagacaaacacagaatggggagcagaggtaggattctcatatggcatcttactggagagttcaatcactggctatcttttycactgcagaggatggaaatcagcatatctttacccaaagacaccatgtttcttagggtgtgccccaactgacatcaaggaaggaatgctccaattggtgaagtggttgtctgaatactgcttgctrggattctctaaatacagccctttcacttatggcttttcaagaatgcccattatgcctaccttagtctattgcttcttgacaacwacaaccctttattccattgtcttcatcctttatggcattgtcccccaagtttgcttcttaaaaggaatacccgtgtttccaaaggtcacagacccttggtttgcagtgtttgcaacactgtatatatccacccagattcaacatttgatagaggtcctttctggtgatggctctgtggcaatgtggtgggatgaacagkgaatctggattctgaagtcagtcactagcgtgttcgcaatcatagaggcagctaagaaagggttaggattgaacaagaagaaattcatgttgtcaaacaaagcaattgacaaggagaagctcaagaagtatgagcaaggtaggtttgatttccaaggtgcagctctgttcatgtccccaatggttgtgttgctcatagtgaacgttgtttccttcattggtggcatatggagactattcaatgcaaaggatattgaagatatgtttggtcagcttttcctagttagttatgtaatggcccttagttatcccatttttgaagggataataaccatgaaaagcaagagtggatag

SEQ ID NO: 81 - protein, 695 aa, Glycyrrhiza uralensis
  VARIANT 302: note = misc_feature - Xaa can be any naturally occurring amino acid
  VARIANT 375: note = misc_feature - Xaa can be any naturally occurring amino acid
  VARIANT 442: note = misc_feature - Xaa can be any naturally occurring amino acid
  VARIANT 579: note = misc_feature - Xaa can be any naturally occurring amino acid
MASFTLHTETVQSWLLLSRLHILLHLAVVLLLLYYRITRFPFHAPTLPWTLMTVGEAIMAVLWFFNQAFRWRPVSRSVMTEKLPSDAKLPGLDIFVCTLDPEKEPTVEVMNTLVSALAMDYPPDKLSVYLSDDGAAPVTLYGVREASEFARVWVPFCKKYGIKSRCPKVFFSPSAEDEHLLRTDEFRSERDLIKAKYEKMQKNIEKFGSDAKNCRMVTDRPPRIEILIDQPDMPRVVYVSRERRPSLPHKFKGGALNTLLRVSGLISNGPYVLVVDCDMYCNDPSSAKQAMCFFLDPETSKXIAFVQFPQMFHNLGKKDIYDNQSRTAFKTMWQGMDGLRGPGLSGSGNYLNRSALLFGSPNQKDDYLDDAQNYXGKSTMYIESLKAIRGQKTMKKNISRDEILREAQVLASCSYETNTEWGAEVGFSYGILLESSITGYLXHCRGWKSAYLYPKTPCFLGCAPTDIKEGMLQLVKWLSEYCLLGFSKYSPFTYGFSRMPIMPTLVYCFLTTTTLYSIVFILYGIVPQVCFLKGIPVFPKVTDPWFAVFATLYISTQIQHLIEVLSGDGSVAMWWDEQXIWILKSVTSVFAIIEAAKKGLGLNKKKFMLSNKAIDKEKLKKYEQGRFDFQGAALFMSPMVVLLIVNVVSFIGGIWRLFNAKDIEDMFGQLFLVSYVMALSYPIFEGIITMKSKSG

SEQ ID NO: 82 - DNA, 1445 bp, Glycyrrhiza uralensis
  source 1..1445: mol_type = unassigned DNA
atgaccatgggtaacgagaatcgggagctgcacataatcttcttcccctttctggcgaacggccacatcatcccctgcgtggacttggccagagtcttcgccgcaagaggaatcagagccaccatagtcaccacccacctcaacgttccctacatttccagaaccatcggaaaagccaacatcaacatcagaaccatcaagttcccttccaccgaagactctggccttcccgaaggctgcgagaataccgagtcagcactcgcccctgacaagttcatcaagttcatgaaggccaccctgctcctgagggacccacttgaacacgtgttacaggaagagcaaccacactcttggtcgccgacatgttcttcccttgggccaccgactccgccgcaaaattcggcatccctaggatcgtgttccacggcctcggttacttcccactctgcgttcttgcatgcacgagacagtacaagcctcaggacaaggtttcatcttacacggaacccttcgtggttccgaatctcccgggtgaaataacactgacgaagatgcagctgccgcagttgcctcagcacgacaaggtcttcacccagttgttggaagagtcaaacgaatcggagttgaagagcttcggtgtgattgtaaacagcttctacgaacttgaaccggtttacgcggatcattacaggaacgagcttgggagaagagcttggcatttgggtccggtttcattatgcagtagggacacggaggaaaaatcgcggaggggaagggaagctgcaattgatgagaacgagtgcttgaagtggcttcaatcaaaggaacccaattcggttgtttatgtttgtttcggtagcatgatggttttcagtgacgctcagctaaaagagattgcgatgggtcttgaggcttcagggaagccattcatatgggtggtgaagaaaggaggggctaaaagtgaaggtgagaaattggagtggcttccagaagggtttgaggagagaatgggggaaagtaataagggactaatcataaggggttgggcaccacaggtgatgattttggaccatggagcggttggagggtttgtgacacattgtgggtggaattcaacgctggaaggagtgtgtgcaggggtgccaatggtgacttggcccatgtatggggaacaattttacaacgccaagtttctgacggacatagtgaaaattggggtgggtgttggggttcaaacgtggattgggatgggaggaggagagcctgtgaagaaggaagtgatagagcaggcagtgagaaggataatggtggggcaggaagcagaggaaatgagaaacagagccaaggaactgagccagatggcaaagcgtgctgtggaggaaggaggatcgtctcacaacgattttaactctttaattgaggatttgaggtcgcgtgcccattaa

SEQ ID NO: 83 - protein, 481 aa, Glycyrrhiza uralensis
MTMGNENRELHIIFFPFLANGHIIPCVDLARVFAARGIRATIVTTHLNVPYISRTIGKANINIRTIKFPSTEDSGLPEGCENTESALAPDKFIKFMKATLLLRDPLEHVLQEEQPHCLVADMFFPWATDSAAKFGIPRIVFHGLGYFPLCVLACTRQYKPQDKVSSYTEPFVVPNLPGEITLTKMQLPQLPQHDKVFTQLLEESNESELKSFGVIVNSFYELEPVYADHYRNELGRRAWHLGPVSLCSRDTEEKSRRGREAAIDENECLKWLQSKEPNSVVYVCFGSMMVFSDAQLKEIAMGLEASGKPFIWVVKKGGAKSEGEKLEWLPEGFEERMGESNKGLIIRGWAPQVMILDHGAVGGFVTHCGWNSTLEGVCAGVPMVTWPMYGEQFYNAKFLTDIVKIGVGVGVQTWIGMGGGEPVKKEVIEQAVRRIMVGQEAEEMRNRAKELSQMAKRAVEEGGSSHNDFNSLIEDLRSRAH

SEQ ID NO: 84 - DNA, 1521 bp, Glycyrrhiza uralensis
  source 1..1521: mol_type = unassigned DNA
atggactcctttggggttgaaggtgatcaccaagccgacaccacagtgctgaaggcggtttttcttcccttcatctcaaaaagtcatctcatccgtgaggtggacaaagcaaggatcttcgccatgcacggcgtggatgtcaccatcatcaccacgccggccaacgctgccactttccaaacctccattgaccgcgactccagccgcggccgctccatcagaacgcacatcgttccgttcccccaagtccccggtctaccacagggactcgagagactcgacgccgacactcctcaacacttgctctccaagatctaccatggactatccattctgcaagagcagttccaacaactgttccgtgaaatgaggccagatttcatagtcactgacatgtactacccttggagcgtcgatgccgccgccgagttggggattccgaggttggtttgtaacggtggaagctacttcgctcagtcagctgttaactccgttgagctattttcaccacaagccaaggttgattcaaataccgagacttttctgcttcctgggttaccccatgaggttgagatgacacgtttgcaactaccggattggcttagaggagcaccgaatgagtacacctatttgatgaagatgatcaaggattcagagaggaagagttatgggtcattgttcaatagcttttatgagcttgaagggacttatgaggaacattacaagaaagccatgggaaccaagagttggagtgtggggccagtttctttgtgggtgaaccaagatgcttctgataaggcttgtaggggggatgttaaagaaggaaaaggagatggggtggtgcttacttggctggattctaaaacagaggactctgttttgtatgtgagttttgggagcatgaacaagttccctaaaactcagcttgttgagatagctcatgccctcgaagattctggccatgatttcatttgggtcgttggcaaaattgaagaaggtgaaggtggtgctgattttttgagggaatttgagaagaaagtgaaagaaaaaaacagaggttatctgatatggggttgggcaccacagcttctgattctggagcatcctgcggttggagcagtggtgactcattgtgggtggaacaccgttatggaaagtgtgaatgcaagtttgccattggcaacttggccattgtttgcggagcagttcttcaatgagaagctagtggttgatgtggtgaagattggtgtgccagttggggttaaggaatggagaaattggaatgagtttggggatgaggttgtgaagagggaggacataggaaaggccattgcttttttgatgggtggtggggatgaatccttggaaatgaggaagagggtcaaggtgctcagtggtgctacaaagaaagctattcaggttggtgggtcttctcacaccaagttgaaagaactcatagaagagctcaagtcaatcaagctacaaaaggtcaacaacaaattaatggaggcagtggcttaa

SEQ ID NO: 85 - protein, 506 aa, Glycyrrhiza uralensis
MDSFGVEGDHQADTTVLKAVFLPFISKSHLIREVDKARIFAMHGVDVTIITTPANAATFQTSIDRDSSRGRSIRTHIVPFPQVPGLPQGLERLDADTPQHLLSKIYHGLSILQEQFQQLFREMRPDFIVTDMYYPWSVDAAAELGIPRLVCNGGSYFAQSAVNSVELFSPQAKVDSNTETFLLPGLPHEVEMTRLQLPDWLRGAPNEYTYLMKMIKDSERKSYGSLFNSFYELEGTYEEHYKKAMGTKSWSVGPVSLWVNQDASDKACRGDVKEGKGDGVVLTWLDSKTEDSVLYVSFGSMNKFPKTQLVEIAHALEDSGHDFIWVVGKIEEGEGGADFLREFEKKVKEKNRGYLIWGWAPQLLILEHPAVGAVVTHCGWNTVMESVNASLPLATWPLFAEQFFNEKLVVDVVKIGVPVGVKEWRNWNEFGDEVVKREDIGKAIAFLMGGGDESLEMRKRVKVLSGATKKAIQVGGSSHTKLKELIEELKSIKLQKVNNKLMEAVA

SEQ ID NO: 86 - DNA, 1563 bp, Arabidopsis thaliana
  source 1..1563: mol_type = unassigned DNA
atgtctctatttctgaagcccttcctcttcctatacgacaccactcttagtcttctcttacttctgttcaatggatggagtcttgaggatacagcagcagcccaaaagaggcgtgaagcagacaaaaatgctgcagaaactgaatggatccaactccaatacttgtggaccaaaacaaggagtgttgtactacttcccgttttcaagggtttggtggttatgtgtttggttctatccattatagtgttcttcgagagtttttacatgaactttgtgatactcttcgtcaagttatttaaacgtaaaccccataaagtgtacaaatgggaggccatgcaagaagatgttgaggttggacccgataactacccaatggttcttatccaaataccaatgtacaatgaaaaagaggaaggtgtggacgtagagattgcaaaatggcaaagccaaggcataaacataaggtgtgaaaggagagataacaggaacggctacaaagccggagctatgaaagaagctcttacgcagagctacgtcaagcaatgcgacttcgtagcagtcttcgatgctgatttccaacccgagcccgattatctcatccgcgctgtccctttccttgtccacaaccctgacgttgctctagttcaagcccgatggatatttgttaacgcgaacaaatgcttgatgacgaggatgcaagagatgtctctcaactatcatttcaaagtggaacaagaatcagggtcgactagacatgctttcttcgggtttaatggaaccgcgggtgtatggagaatatcggcaatggaagcagcaggaggatggaaatcaaggaccacagtagaggacatggacttggctgttcgtgttggtcttcatggctggaaatttgtctaccttaacgacctcacggtgagaaacgagcttccaagcaaatttaaggcctacagattccagcaacataggtggtcctgtggaccggcgaatctatttagaaaaatgacgatggagatcattttcaataagagagtatcaatttggaagaagttttatgtgatctacagctttttcttcgtaaggaaagtggcggtacacttcttgacattcttcttctactgtataattgtgccaacaagtgtcttcttccctgaaatccacatcccatcttggtctaccatttacgttccctctttgatcagtatcttccacaccctggcaactccaagatccttctacctcgtgatattttgggtcttgttcgagaatgtaatggctatgcatcgaaccaaaggtacgtgcattggcctacttgaaggaggaagagtaaacgaatgggttgtgaccgaaaaactaggagatgctttgaagagtaagctactctctcgggtagtccaaagaaaatcttgttatcaaagagtgaattccaaggaagtgatggtgggggtatacatattaggatgtgcactctatggcctgatctatgggcacacatggttacatttctatctttttcttcaggccacagcctttttcgtctccggttttggttttgtcggaacctaa

SEQ ID NO: 87 - protein, 520 aa, Arabidopsis thaliana
MSLFLKPFLFLYDTTLSLLLLLFNGWSLEDTAAAQKRREADKNAAETEWIQLQYLWTKTRSVVLLPVFKGLVVMCLVLSIIVFFESFYMNFVILFVKLFKRKPHKVYKWEAMQEDVEVGPDNYPMVLIQIPMYNEKEEGVDVEIAKWQSQGINIRCERRDNRNGYKAGAMKEALTQSYVKQCDFVAVFDADFQPEPDYLIRAVPFLVHNPDVALVQARWIFVNANKCLMTRMQEMSLNYHFKVEQESGSTRHAFFGFNGTAGVWRISAMEAAGGWKSRTTVEDMDLAVRVGLHGWKFVYLNDLTVRNELPSKFKAYRFQQHRWSCGPANLFRKMTMEIIFNKRVSIWKKFYVIYSFFFVRKVAVHFLTFFFYCIIVPTSVFFPEIHIPSWSTIYVPSLISIFHTLATPRSFYLVIFWVLFENVMAMHRTKGTCIGLLEGGRVNEWVVTEKLGDALKSKLLSRVVQRKSCYQRVNSKEVMVGVYILGCALYGLIYGHTWLHFYLFLQATAFFVSGFGFVGT

SEQ ID NO: 88 - DNA, 2274 bp, Arabidopsis thaliana
  source 1..2274: mol_type = unassigned DNA
atggcggattcaagcttttctcttcctcctctttgtgaaaggatctcatacacgaactattttctaagagctgtatatctcacggttctaggccttttcttttctcttctcttgcaccgaatccgacatacgagcgaatacgacaacgtttggctcgtggctttcttttgtgaatcttgtttcttcttggtatgtctgcttattacttgcctaaaatggagtcctgctgatactaaaccctttcctgatagacttgatgaaagggttcatgaccttccttcggtggatatgttcgtgcccacagcagatccggttcgagagccaccgattatggttgtggacaccgtgctttcgctgttagctgtaaattatccggcaaataaactagcttgttatgtgtcggacgatggatgctcacctctcacttatttctctctcaaggaagcttctaagttcgccaagatttgggtaccgttctgcaaaaagtacaacactagagttagagctccttctagatattttctgaaacctataagcgtcgcaacagaggattatgaattcaatagagactgggaaaagacgaagagggagtacgagaagttgaggcggaaagtggaagatgccaccggagattctcatatgttggatgtagaagatgattttgaagcattctcaaacacaaaaccaaatgatcattcaactctagttaaggtggtatgggagaacaagggaggtgtaggagacgagaaagagatccctcatatcatatacatatcaagagagaaaagaccaaattatgttcataatcaaaaatgtggagccatgaactttctggcaagagtgtcagggttgatgacaaacgcaccatacatcttgaacgtggattgcgacatgtatgccaatgatgcagatgtagtccgacaagcaatgtgtatacttctgcaagaatcattaaatatgaaacattgtgcttttgttcaattccgtcaagaattctatgattcaagcaccgagctaatagtcgtcctacaatcacatttgggacgaggaatcgcgggaatccaaggaccgatatatataggatcaggatgcgtccacacgagaagagttatgtatggtttatctccagacgatttcgaagttgatggaagtctttcttcagttgctacaagggagtttttggttaaggatagtttagcgagaagatttggtaattctaaagagatgatgaaatcagtggttgatgcaatacaaagaaatccaaatccacaaaatatacttacaaactccatagaagcggctcgagaagtgggacattgtcagtacgagtaccaaaccagctggggaaacaccatcggctggttatatgattcagtggcggaagatttaaacacgagtatcggaatacattcgagaggttggactagctcatacatttctccggatacacctgcatttcttggatctatgccggcaggagtacccgaggcgttactccagcagcgtcgatgggcgacaggatggatcgaaatccttttcaacaagcaaagtccgttgcgaggattgtttagcaagaaaataagattccgacaacgattagcttatctttgcattatcacctgtctaaggtcaatccctgagcttatttattgtctccttcctgcttattgcctactccacaactctaccttattccccaagggactttatttaggcataactgtcacacttgttgggatacattgtctctatactctatgggaatttatgagccttggttattccgtacaatcgtggctagtctcccaatcagtttggagaatagtagccactagtagttggttatttagcatctttgatatcacactcaagcttcttggcatctcggaaacggtgttcataatcactaaaaagactgtggctgggaccaagtcagcattagggtctggaccctctcaaggagaagacgttggtccaaactcagacttgtttaaatttgaatttgatggctcactttgtttcttgcctggcacatttattgtgttggtgaatatagccgctctagctgttttttctgtgggtctacaacggtcgagttacagccatgaaggaggtggttcgggtctggcagaggcttgcggatgtgttttggtaatgatgttgttccttccatttctaatgggtttgtttaagaaaggaaaatatggaaccccattgtctactctctctatagctggctttttagcagttttatttgttgttttctctgtttga

SEQ ID NO: 89 - protein, 757 aa, Arabidopsis thaliana
MADSSFSLPPLCERISYTNYFLRAVYLTVLGLFFSLLLHRIRHTSEYDNVWLVAFFCESCFFLVCLLITCLKWSPADTKPFPDRLDERVHDLPSVDMFVPTADPVREPPIMVVDTVLSLLAVNYPANKLACYVSDDGCSPLTYFSLKEASKFAKIWVPFCKKYNTRVRAPSRYFLKPISVATEDYEFNRDWEKTKREYEKLRRKVEDATGDSHMLDVEDDFEAFSNTKPNDHSTLVKVVWENKGGVGDEKEIPHIIYISREKRPNYVHNQKCGAMNFLARVSGLMTNAPYILNVDCDMYANDADVVRQAMCILLQESLNMKHCAFVQFRQEFYDSSTELIVVLQSHLGRGIAGIQGPIYIGSGCVHTRRVMYGLSPDDFEVDGSLSSVATREFLVKDSLARRFGNSKEMMKSVVDAIQRNPNPQNILTNSIEAAREVGHCQYEYQTSWGNTIGWLYDSVAEDLNTSIGIHSRGWTSSYISPDTPAFLGSMPAGVPEALLQQRRWATGWIEILFNKQSPLRGLFSKKIRFRQRLAYLCIITCLRSIPELIYCLLPAYCLLHNSTLFPKGLYLGITVTLVGIHCLYTLWEFMSLGYSVQSWLVSQSVWRIVATSSWLFSIFDITLKLLGISETVFIITKKTVAGTKSALGSGPSQGEDVGPNSDLFKFEFDGSLCFLPGTFIVLVNIAALAVFSVGLQRSSYSHEGGGSGLAEACGCVLVMMLFLPFLMGLFKKGKYGTPLSTLSIAGFLAVLFVVFSV

SEQ ID NO: 90 - DNA, 2190 bp, Arabidopsis thaliana
  source 1..2190: mol_type = unassigned DNA
atggtaaacaaagacgaccggattagaccggttcatgaagccgacggtgaaccgctttttgagactaggagaagaaccggtagagtgattgcgtaccggtttttctcagcctcggttttcgtgtgtatctgtttgatttggttctacagaattggtgagattggtgataaccgtaccgttttagatcgattaatctggtttgttatgtttattgtggagatttggttcggtttatattgggtagtcacacaatcttcccggtggaatccggtttggcgatttcccttctccgatagactctctcggagatacggaagcgaccttccgaggctcgacgtcttcgtttgcacggcggatccggtgattgagccgccgttgttggtggtaaacacagtcttatctgtgacggctcttgactacccaccggagaaactcgccgtttatctctcagatgacggtggttctgagctgacgttctatgctctcacggaggcagctgagtttgctaaaacttgggttcccttctgcaagaagttcaacgttgagccaacatctcccgctgcttacttgtcttccaaggcaaactgtcttgattctgcggctgaggaggtggctaagctgtatagagaaatggcggcgaggattgaaacggcggcgagactgggacgaataccggaggaggcgcgggtgaagtacggtgacgggttttcacagtgggatgctgacgctactcgaagaaaccatggaaccattcttcaagttttggtagatggaagagaagggaatacaatagcaataccaacgttggtgtatttatcaagagaaaagagacctcaacatcatcataacttcaaggctggagcaatgaacgcattgctgagggtttcttcgaaaattacttgtgggaaaatcatactaaacttggactgtgatatgtacgcaaacaactcaaagtcaacacgcgacgcgctctgcatcctcctcgatgagaaagagggaaaagagattgctttcgtgcagtttccgcagtgttttgacaatgttacaagaaatgatttgtatggaagcatgatgcgagtaggaattgatgtggaatttcttggattggatggaaatggtggtccgttatacattggaactggatgctttcacagaagagatgtgatctgtggaagaaagtatggagaggaagaagaagaagaagaatctgagagaattcacgaaaatttagagcctgagatgattaaggctctcgcgagctgcacttatgaggaaaacactcaatggggaaaggagatgggtgtgaaatatggttgcccggtagaggatgtaataactggtttgacgattcagtgtcgcggatggaaatcagcctacctgaacccggaaaagcaagcatttctcggggtagcgccgaccaatttgcatcaaatgctagtgcagcagaggagatggtcagagggagactttcagattatgctttcgaagtatagtccggtttggtatggaaaaggaaagatcagtttaggactgatacttggttactgttgctattgtctttgggctccatcttcactacctgtgctcatttactctgttttgacttctctctgtctcttcaaaggcattcctctgtttccaaaggtctcgagctcgtggtttattccgtttggatacgtcactgttgcagctaccgcatatagcctagccgagttcttgtggtgcggagggacgttccgtggatggtggaacgagcaaaggatgtggctttatagaagaacaagctcgtttcttttcggatttatggacacgattaagaagctacttggagtttctgagtctgcgtttgtgatcacagcaaaagtagcagaagaagaagcagcagagagatacaaggaagaggtaatggagtttggagtggagtctcccatgtttctcgtcctcggaacactcggtatgctcaatctcttctgcttcgccgcagcggttgcgagacttgtttccggagacggtggagatttgaaaacaatggggatgcaatttgtgataacaggagtactagttgtcataaactggcctctgtataaaggtatgttgttgaggcaagacaaaggaaagatgccaatgagcgttacagttaaatcagttgttttagctttatctgcctgtacctgtttagcgtttttgtaa

SEQ ID NO: 91 - protein, 729 aa, Arabidopsis thaliana
MVNKDDRIRPVHEADGEPLFETRRRTGRVIAYRFFSASVFVCICLIWFYRIGEIGDNRTVLDRLIWFVMFIVEIWFGLYWVVTQSSRWNPVWRFPFSDRLSRRYGSDLPRLDVFVCTADPVIEPPLLVVNTVLSVTALDYPPEKLAVYLSDDGGSELTFYALTEAAEFAKTWVPFCKKFNVEPTSPAAYLSSKANCLDSAAEEVAKLYREMAARIETAARLGRIPEEARVKYGDGFSQWDADATRRNHGTILQVLVDGREGNTIAIPTLVYLSREKRPQHHHNFKAGAMNALLRVSSKITCGKIILNLDCDMYANNSKSTRDALCILLDEKEGKEIAFVQFPQCFDNVTRNDLYGSMMRVGIDVEFLGLDGNGGPLYIGTGCFHRRDVICGRKYGEEEEEEESERIHENLEPEMIKALASCTYEENTQWGKEMGVKYGCPVEDVITGLTIQCRGWKSAYLNPEKQAFLGVAPTNLHQMLVQQRRWSEGDFQIMLSKYSPVWYGKGKISLGLILGYCCYCLWAPSSLPVLIYSVLTSLCLFKGIPLFPKVSSSWFIPFGYVTVAATAYSLAEFLWCGGTFRGWWNEQRMWLYRRTSSFLFGFMDTIKKLLGVSESAFVITAKVAEEEAAERYKEEVMEFGVESPMFLVLGTLGMLNLFCFAAAVARLVSGDGGDLKTMGMQFVITGVLVVINWPLYKGMLLRQDKGKMPMSVTVKSVVLALSACTCLAFL

SEQ ID NO: 92 - DNA, 2283 bp, Arabidopsis thaliana
  source 1..2283: mol_type = unassigned DNA
atggagactcatagaaagaactcggtcgtcggcaacatcctccacacgtgtcatccttgccggcgcaccattccatatagaatctacgccatatttcacacgtgtggcatcatagctctcatgtatcaccatgtacactcacttgtcacagcaaacaacactcttataacatgtcttcttctcctctccgatattgttctcgccttcatgtgggcaaccacaacttccctccgcttaaacccggttcatcggaccgagtgcccggagaaatatgcagctaaaccggaggactttccaaagctggacgtgtttatatgcacggctgatccgtacaaggagcctccaatgatggttgttaacaccgctttatcggtgatggcttacgagtatccgtcagataagatctcggtgtatgtatcggacgatggaggatcgtcgttgactttctttgctcttattgaagctgctaagttctctaagcagtggttgcccttttgcaagaagaataatgttcaagatcggtctcctgaagtttatttctcttcagagtcacattctcgaagtgatgaagctgaaaaccttaagatgatgtacgaagacatgaagagtagagtagaacatgtggtggagagtggaaaagttgaaactgcgtttatcacatgcgaccaatttcgtggggtattcgatttgtggaccgacaaattcagtcgtcatgaccatcccacaattattcaggtgttgcaaaatagcgagacagatatggacaataccagaaaatatataatgccaaacctaatctatgtttcaagagagaagagtaaagtttcaccacatcatttcaaagctggtgctcttaatactttgctacgagtatcaggggtgatgacaaattcaccgatcattctaacactagactgtgatatgtattcgaacgacccggcaacactggttcgtgctttgtgctatttaacagatcctgaaatcaaatccggtttaggatatgtgcagtttcctcagaaatttctaggaataagcaaaaatgatatatatgcttgtgaaaacaaacgcctcttcattattaatatggttgggtttgatggtctaatgggtccaactcatgtgggaactggttgtttctttaatcgacgagctttctatggacctccatatatgttgattttaccggagataaatgaactaaagccttatcggattgcggataagtctatcaaagcccaagatgttttgtcattagcacacaatgtagcaggatgtatctatgagtacaataccaattggggatccaagattggattcagatatgggtcattagtagaagactactacacagggtttatgctccattgtgaaggatggagatcagtattttgcaacccaaaaaaagctgcattttatggagattccccaaagtgcctagttgatcttgtgggtcaacaaatccgttgggcagttgggcttttcgaaatgtccttttcaaagtatagcccaattacctatggaatcaagtcactggaccttttaatgggtttaggttattgcaactctccgtttaagccattttggtcaattcctctgaccgtctatggacttttaccacagcttgcactcatttctggagttagtgtcttccccaaggcatctgatccgtggttttggctttacatcattttattctttggggcttatgcccaagatctatcagactttttattggaaggaggaacttatcggaaatggtggaacgatcaaagaatgttgatgataaaaggactctcttcattcttctttggttttatagagttcattctcaaaaccctaaacctctccacacctaagttcaacgtcaccagtaaagccaatgatgatgacgaacagaggaagcggtacgagcaagaaatctttgatttcggaacctcttcgtccatgttcttgcccttgaccacggttgccatagtgaatctgcttgcttttgtctgggggctttatggtattctcttctgcggaggagaactctaccttgagctgatgctggtgagcttcgcggtggtgaattgcttaccgatctacggggctatggtgttgaggaaagatgatggaaaattatcaaaaagaacttgtttcttagctgggaacctccacgttggttcttattgtgtcaagttacttcgtcctcaagtaacttcaccccttaggttaattcacaacaataatacgtctggctggttcaagcggaagaaacacaatatgaatgaatctgtgtaa

SEQ ID NO: 93 - DNA, 2088 bp, Spinacia oleracea
  source 1..2088: mol_type = unassigned DNA
atggcaacttctcacattcgcaatgtccaattaaccagagccattgttaaccgtctccacatcttcctccattccgtagccatcttctcgctcttctactaccgtttcacttccttcttcaactccgacatctccatacttgcttactccttactcaccaccgccgaactcttcttaacctttctatgggcttttactcaggctttccggtggcgtcccgtaatgagggaagtctccgggtacgaatccatcaaacccgaacaactaccgggtttggatgtcttcattgtcactgctgacccgacaaaggagccagttctggaggtgatgaactccgtgatatcatccatggcgttggattatccggttgatagactggcggtttacttgtcggatgacggtggttctccgttgtcgaaggaggcgattaagaaggcttatgagtttgctaagctttggattcctttttgtaataagtataatgttaagacaaggtgtcctcaggctttcttctcgcctcttgctgatggggaaaggcttgattggaattctgagtttatggctgatcaattggaactccagaccaaatatgaagcttttagaaactatgtggagaaagaaagtggagataacaccaaatgtactgcagttcatgatcgacctccttgcgttgagattatacatgacaacaaacagaacggagaaagtgatgtgaagatgccccttctggtttatgtagccagggaaaagagacctggtcgtcctcatcgtttcaaagctggagcccttaatgctcttcttcgagtatccagtttaatgagcaatgcaccttacttattggtgttggattgtgatatgtactgccatgatccaacttctgctcgtcaatctatgtgcttccatcttgacacaaacatggcttcctctcttgcatatgtgcaataccctcaaattttctataatgttagcaaaaatgacatctatgatggccaagccagatcagctcatatgacgaaatggaaaggcatggatggactcagaggcccggtcttgaatggaactgggtattatttgaagcgaaaagcattatttggaaagcctaataacgaagatgaatacctcaacagtcaaccagaaaaggcctttggctcctccacaaaattaattgctgcactaagagagaactccaagcaaaatcttgccataaaggaattgacagaagatgagttgtaccaagaggctagaaatttggctacttgcacatatgaagcaaacacactatggggcagtgaggtaggatattcgtatgagtgcttgttggagagtacattcactggatatatgttacattgcagaggatggaaatctgtgtatctttacccaaaaagaccatgcttcttgggatgcacaacgattgatatgaaggatgctacggttcaactaataaaatggacctcctcattacttggaattgccctgtcgaagtctagccctctaactttggccatgtccagtatgtcaatcctgcaaagcatgtgttacgcgtacatcacatttacaggcctttttgcagctccattggttatatatggtgttgtccttccaataagcctattgaagggctttcctattttccctaaggtatcggatccatggattttgccatttgtgttgatatttgtatcctcccatcttcaacatctatatgaggtcctggaaagtgacaaatcagcaacacaatggtggaatgaggtgagaatttggatgatgaaatcagtgacagcctgtttgtttgggttgacggaagcgataatgaagaagattggagtacaaactgcaacattcagattaacaaataaggtagttgagaaggaaaagatggataaatacgagaaggagaggtttgatttctcaggagcagctatgcttatggttcctcttaatattttggtggtactaaatatggtgtcattcattggtggactcatgagggtcataatcaacaacagttatgatcaaatgtttgcacaacttttcctctccttttttgtcctacttcttagctaccctgttgttaagggatggtta

SEQ ID NO: 94 - protein, 704 aa, Beta vulgaris
MSSLHICKVQTTRAILSRFHILFHSLAILALFYYRFTSFSTTKSGILPWTLLTTAEVVLGFVWALTQAFRWRPVLRDVAGWDSIKEEQLPGVDVFICTADPIKEPVLEVMNTVLSAMALDYPAEKLGVYLSDDGGSPLTREAIKEASKFAKVWLPFCSKYGIKTRCPQAFFSSFCDGERLDWNQDFKADELVLKSKYEAFKNYVEKASEDESKCTMAHDRSPCVEIIHDNKQNGEGEVKMPLLVYVSREKRPNRPHRFKAGALNALLRVSGVLSNGPYLLVLDCDMYCNDPTSARQSMCFHLDPKLAPSLAFVQYPQIFYNTSKNDIYDGQARSAYKTKWQGMDGIRGPVLTGTGYYLKRKALYGQPHNEDEFLINQPEKAFGSSTKFIASVSSNSKQNMALKEMTRDDLLEEAKNLATCAYESNTEWGNKIGYSYECLLESTFTGYLLHCKGWISVYLYPKRPCFLGCTTIDMKDAMVQLMKWTSGLLGVGISKFSPLTYAFSRMSILQSMCYGYFTFSALFGVSFLIYGIVLPVCLLKGVPVFPKVSDPWIGVFVVVFASSLLQHLYEVLSSDDSIKTWWNEIRIWIIKSVTASLFGTMDAIMKKIGIQKASFRLTNKVVDKEKLEKYEKGKFDFQGAAVFMVPLIILVVLNMVSFVGGLRRAIINKNCDEMFGQLFLSFFLLVLSYPVLEGIVTKVRKGRD

SEQ ID NO: 95 - DNA, 2656 bp, Beta vulgaris
  source 1..2656: mol_type = unassigned DNA
catcacagccacatgggaaacaaaaagtcttaacctcggccatctcgtgtgcctcgtgcacattcaacattgattttaattgtgtgctttctccaaaatgtaccatactatatcatcttgtcagaattccaaactccaactataaccaccattgaagtcatctacacacaaacacacacactctttctccctaaaaatgtcttctctccacatttgcaaagtccaaacaacaagagcaatacttagccgtttccacatactcttccactccttagccatccttgctttattctactaccgttttacatcgttctctaccaccaaatcaggcatacttccatggaccttactaaccacagcagaggtggttctaggctttgtatgggcgttaacacaggcctttcgatggcggcctgtgttgcgagatgtagctggatgggattccatcaaggaggaacaactgccaggggtggacgtgttcatatgcacagctgatccaataaaggagccggtgttagaggtgatgaacacggtgctttcggcgatggcattggattacccggcagagaagttgggtgtttatctttcggatgatggaggttctcccttgactagggaggctattaaggaggcttctaagtttgctaaggtttggcttcctttttgtagtaagtatggtatcaagactaggtgtcctcaggctttcttctcttctttttgtgatggggaaagacttgattggaatcaggactttaaggctgatgaattggtgctcaagtcaaaatatgaagcttttaagaattatgtggagaaagcaagtgaagatgaaagcaaatgcaccatggcacatgatcgttccccttgcgttgagattatacatgacaacaagcaaaatggagaaggcgaagtgaaaatgccccttttggtctacgtatccagggaaaagagaccaaatcgtcctcatcgtttcaaagccggagctcttaatgctcttctcagagtatcaggtgtattgagcaacgggccttacttattggtgttggactgtgatatgtactgcaatgatccaacttctgctcgtcaatctatgtgctttcatcttgacccaaaattggctccttcacttgcatttgtgcaatacccacaaattttctacaacaccagtaaaaatgatatctatgatggccaagctagatccgcgtacaagacaaaatggcaaggaatggatggtattagaggaccagtcttgacaggaacagggtattacttgaagaggaaagcattgtatggacaacctcataacgaagatgaatttctcattaatcaaccagagaaggccttcggctcctccacaaaattcattgcgtcagttagttcaaactccaagcaaaatatggccttgaaggaaatgacaagagacgacttgttagaagaggctaaaaatttggctacttgtgcatatgaatcaaacactgaatggggtaacaagattggatattcgtatgagtgtttgttggagagtacatttaccggatatctcttacattgcaaaggatggatttccgtgtatctttacccaaaaagaccctgcttcttaggatgcacgacgattgacatgaaagatgccatggttcaactaatgaaatggacctctggattactaggagttggcatatcaaagtttagccctctaacttatgccttttcgaggatgtctatattacaaagcatgtgctacggttacttcacattttcagcccttttcggagtttcgttcttaatatatggcatcgtccttccagtatgcctattgaagggtgttcctgtttttcctaaggtatcggatccatggattggagttttcgtggtagtatttgcatcctccctccttcaacatttatacgaggttctctcaagtgacgattccattaaaacatggtggaacgagatcagaatttggatcatcaaatcggtaacagcttccttatttggaacaatggatgcaataatgaaaaagatcggcatacaaaaggctagtttccgattaactaacaaggttgtggacaaggaaaagctcgaaaaatatgagaagggcaagtttgatttccaaggagcagctgtgttcatggttcctcttatcattttagtggtactaaatatggtgtcatttgttggcggattaagaagggcaataatcaacaagaattgtgatgaaatgtttgggcaacttttcctctcattctttctcttagttcttagctaccccgttttagaagggatagtaacaaaagtaagaaaaggacgtgattgagatgaatttgcattgtttggtaaaagatccaaacttagagaaagagattgcgtaggagatcaaaggaaacaatgtgagagatttacaggcttcatgaggcttaagacctcattaatttttgtgacaatttacaaattctgtctctatattttggtcaagacgtatcatttgaaaatttccatggttaggtagttagatttcaatgttccacgtttgtaaataagaagataaaataaggaaatttgtgattttagctttacatttctatgagatagtcctttgttgtgatgaaagttgtgttcttagtgaaaataataaaacgtgacatcaaaattttgagtatat

SEQ ID NO: 96 - protein, 706 aa, Chenopodium quinoa
MAATHICKVQTKRVIINRIHILFHSLAILALFYYRFSSFSNPHISLFPWVLLTIADLVFTFIWAMTQAFRWRPVLHDVSGYESINPRDLPKIDIFICTADPTKEPVLEVMNSVISSMALDYPPEKMAVYLSDDGGSPLTREAIKKAVEFAKVWIPFCNMYGIKTRCPDAFFSALGNDERLHRDQDFNAHESLLKSKYEAFKKYVEKESGDINKCTVVHDREPCIEIIHDSKQDGEAEVKMPLVVYVAREKRPGHPHRFKAGALNALLRVSGLLSNAPYLLVLDCDMYCHDPTSARQSMCFHLDPNMSPSLAFVQYPQIFYNTSKNDIYDGQARSAHTTKWQGMDGLRGPVLNGTGYYLKKKAIYGRPHNEDEYLINEPEKAFGSSTKFIASLKENSNQDLVLKEFTNDLLQEARNLATCTYEANSLWGVEVGFSYDCLLESSYTGYLLHCKGWRSVYLYPKRPCFLGCTTIDMKDAIVQLIKWTSGLLGVAMSKFSPLTYAMSRMSILQSMCYAYITCSGLLAVPLFIYGVVLPFCLLKGVPVFPKVSDPWMLGFVFVFVSSHVQHLFEVLASDHSVQQWWNEVRIWIMKAITACLFGSTEAIMKKIGIQKTTFRLTNKVVEKEKLDKYEKGKFDFSGAAMLMVPLIILTILNLVSFVGGLVRVINHNNYDDMFGQLFLSFYLLLLSYPTFEGIVTKVTDKLRKKE

SEQ ID NO: 97 - DNA, 2524 bp, Chenopodium quinoa
  source 1..2524: mol_type = unassigned DNA
aaaagtgaaactggtgacttagtcttgtgtcacccgggttcctcgagcacatattcttcaagattttggttttttgtacggagtatttatatacacaaaaattaggaaccaaataggaagactcatatcatttcaaaatggcggcaacacacatttgcaaagtccaaaccaaaagagtcattatcaaccgtattcatatcctctttcactctttagccattcttgctctcttctactaccgtttctcgtctttctccaaccctcatatctccctttttccatgggtattattgactatcgccgacctcgttttcaccttcatttgggccatgactcaggccttccgttggcgccccgtcttgcacgatgtgtctggctatgagtccatcaatccacgcgatcttccaaagatcgatatttttatatgcaccgctgatcccaccaaggagcctgtgttggaagtgatgaactcggtgatatcatccatggcgctcgattatccgcctgaaaaaatggcggtgtatttgtcggatgatggtggttctcctttgactagagaggctattaagaaggctgttgaatttgctaaggtttggattcctttttgtaatatgtatggtattaagactaggtgtcctgatgctttcttctccgctttgggtaatgatgaaagacttcatcgtgatcaagactttaacgctcatgaatcactcctcaagtcgaaatacgaagcttttaagaaatatgtggagaaagaaagcggtgatattaataaatgcaccgttgtgcatgatcgtgaaccttgcattgagattatacatgacagtaaacaggatggagaagctgaagtgaaaatgccccttgtagtttatgtagccagggaaaagagaccaggtcatcctcatcgtttcaaagctggagcccttaacgctcttctccgagtatcaggactattgagcaatgcgccttacttattggtgttagactgtgatatgtactgtcatgatccaacctctgctcgtcaatctatgtgcttccatcttgacccgaacatgtctccctctcttgcctttgttcaataccctcaaattttctacaacactagtaaaaatgatatctatgatggtcaagccagatcagctcatacgacgaaatggcaaggcatggatggactcagaggaccggtcttgaatggaactgggtattatctgaagaagaaggcgatatatggaaggccccataatgaagatgaatacctcatcaatgaaccagaaaaggcttttggttcttccacaaaattcattgcttcacttaaagaaaactcgaaccaggatcttgtcttgaaggaattcacaaacgatttgttacaagaggctagaaatttggctacttgcacttatgaagcaaactcgctatggggtgttgaggtagggttttcgtatgattgcctgttggagagttcatacactggatatctcttacattgtaaaggatggagatctgtgtatctttatcccaaaagaccgtgcttcttgggatgcacgacaattgacatgaaggatgctattgttcaattaataaaatggacttccggattacttggagttgccatgtcaaagtttagccctcttacttatgccatgtccagaatgtctatattgcaaagcatgtgttacgcgtacatcacgtgttcaggtcttctagcagttccactctttatatatggtgttgttctaccattctgcctacttaagggcgttcctgtttttcctaaggtatcggatccatggatgttgggtttcgtgtttgtatttgtatcctcccatgttcaacatctattcgaagtgctagcaagtgatcattcagtgcaacagtggtggaatgaggtgagaatctggatcatgaaagcgataacagcctgcttgtttggatcaactgaagcaataatgaagaagattgggatacagaaaacaacattcagattaacaaataaggttgtggagaaagagaagttggataaatacgagaagggaaagttcgatttctcaggagcagcaatgctaatggttcctctcatcattttgactatactaaatttggtgtcgttcgttgggggacttgtaagggtgatcaaccacaacaactatgatgatatgttcgggcaacttttcctgtcattttatctcctacttcttagctaccctactttcgaagggattgttacaaaagttacagacaaacttagaaagaaagaataaggagtgattgagtaactgcctagtacagttttcacttcacttctctagattagtccttgtttttgtttatgtttattaagatcagcaacacttgtagacggttgcaataatgagttcaataccgtttgttctgtccctcttgcaagaacaagtatataaatacttttcattagccggttgcattgttggattcatatagatgaatatttcaaatatttcatctttttgaacttacacactaatgatttatatctggaattttgaaa

SEQ ID NO: 98 - protein, 715 aa, Medicago sativa
MATFTFHKETVQPLLPLRRAYIIFHFTCVLFLFYYRISNLFISYPWFLMTIAEIILSFLWFFNQAFRWRLVNRSVMTEKLPPEEKLPGLDIFVCTIDPEKEPTVDVMNTVISAIAMDYPSNKLSIYLSDDGGSPITLFGIKEAFEFAKVWVPFCKKYDVKSRCPKFFFTALGENERLHRPREFEEVRDQIKKRLNRVDPNSSQVENSKEHMPTKAKYEKMQKNIEKFGSNLKNLCMVTDRPSRIEIINDQKEMPLVVYVSREKRPSVPHRFKGGALNTLLRVSGLISNGPYVLVVDCDMNCNDASSAKQSMCFFLDPETSKDVAFVQFPQMFHNLSKKDIYDSQTRTAFTTKWKGMDGLRGPGLTGSGNYISRSALLFGSPNQKGDYLLDALYNFGKSNMYVESLKALRGQQTKKQNISRDVILQEACEVASCSYERNTNWGNEVGFSYAIKLESTVTGYLLHCRGWRSTYLYPKRPCFLGCAPTDMKEGLIQPIKWSSELLLLAISKYSPFTYGLSRLPTIHCLTFCYLVSTTQFATAYILYGFVPQICFLKGIPVYPKVTDPWFIVFTVLYLSSQIHHYIEVISTGGSSMIWWNEQRSGIVKSIGCVFAIIETAKKKFGLNKAKFTLSDKAIDKDKLKKYEQGKFNFDGAALLMAPVIVLLTINIVCFFGGLWRLLNVRDFDEMFGQLFLIIYILALSHPIVEGIISMKRKSG

SEQ ID NO: 99 - DNA, 2148 bp, Medicago sativa
  source 1..2148: mol_type = unassigned DNA
atggcaaccttcacatttcacaaagaaacagttcaaccattgttacctctaagaagagcttacataatcttccacttcacatgtgtcttgtttctcttttactaccgtatcagcaatttgtttatttcatatccatggtttctaatgacaatagctgagattattctatcatttctatggtttttcaaccaagcattccgttggaggctggtgaatcgttcagttatgaccgagaaattaccgccggaggagaagttgccgggactcgacatatttgtgtgtaccattgatcctgaaaaagaaccaacggttgatgttatgaacactgttatttctgctattgcaatggattacccttctaataaactttctatttatctttctgatgatggaggttctcctattactctttttgggatcaaagaggcttttgaatttgctaaagtttgggttcctttttgtaaaaaatatgatgttaagtcaaggtgtcctaagtttttcttcactgctttgggtgagaatgaacgacttcatcgacctcgtgaatttgaagaagtgagggaccagattaagaagagattaaatagagtggatcctaactcatcacaagttgaaaactccaaggaacatatgcccaccaaagccaaatacgagaaaatgcagaaaaatattgagaaattcggaagcaacctaaagaatctttgtatggtgaccgatagaccttctcggatcgagatcattaatgaccaaaaagaaatgccactagttgtttatgtatctcgtgaaaaaagaccatctgttcctcacagattcaaaggaggagctctcaatacattgcttagggtgtcagggctaatcagcaatggaccttatgtacttgtcgtagattgtgatatgaattgtaatgatgcatcatcagccaaacaatccatgtgcttttttcttgatcctgaaacctctaaagatgttgcttttgttcaattccctcaaatgtttcacaaccttagcaagaaagacatatatgatagtcagactaggactgcttttacgacaaagtggaagggaatggatggattaagaggtccaggtctaactggcagtggaaattatataagtagaagtgcattactctttggaagtccaaaccaaaaaggggactatctacttgatgctctatacaactttggcaagtctaacatgtatgtagaatcactaaaagcgttacgtggtcaacaaactaagaagcagaatatttcaagagatgtaattttacaagaagcatgtgaagtggcttcttgttcctatgagagaaacacaaattggggtaatgaggtgggattctcgtatgctataaaacttgagagtaccgttactggctatctcctccattgtagaggatggagatcaacttatctttaccctaaaagaccatgtttcttaggatgtgctccaactgacatgaaagagggattgattcaaccgataaagtggtcatctgaacttttgttgcttgcaatctctaaatatagcccattcacttatggcctttcaagattgcccactattcattgtttaactttttgttacttggtaagcacaacccaatttgcaacagcctacatcttatatggattcgttcctcagatttgcttcttgaagggaatacctgtatatccaaaggttacagatccttggtttatagtgtttacagtattgtatctatccagtcaaattcatcattatattgaggtaatttcaactggtggctcctcgatgatttggtggaatgaacaaagaagtgggattgtaaaatcaattgggtgcgttttcgcaattatagaaacagcgaaaaaaaagtttgggttgaacaaggcaaaattcactttatcggacaaagcaattgacaaagataagctaaagaaatatgagcagggtaagtttaattttgatggtgcagcattgctcatggcaccagtgattgtgttactcacaataaatattgtttgcttctttggtggtttatggagactactcaatgtgagggattttgatgaaatgtttggtcaacttttcctcattatctatatacttgctctaagtcatcctattgtggaggggattatatctatgaagcggaagagtgggtag

SEQ ID NO: 100 - protein, 696 aa, Glycine max
MATFHTETVQSGLALSRLHILFHSVALLFLYYYRISHILLEPSFVWIFMTIAELIFGELWLFKQAFRWRPVSRAVMPEKLPSDGKLPALDIFVCTVDPEKEPTVQVMDTVISAIAMDYPSNKLAVYLSDDGGCPVTLYGIREASRFAKEWVPFCRKYGINSRCPKAFFSPMGEDERELLLLRNHEFLAEQEQLKAKYNIMQKNIDEFGRDPKNRSIVFDRPARIEIINEQSEIPLVVYVSRERRPNVPHTYKGGALNTLLRVSGLFSNGPYVLVVDCDMYCNDPSSAKQAMCFFLDPETSKDIAFVQFPQMFHNLSMKDIYDSQHRHAFTTMWQGMDGLRGPGLSGSGNYLSRSALIFPSPYEKDGYEHNAQNKFGNSTMYIESLKAIQGQQTYKTSISRNVILQEAQAVASCSYEIDTNWGNEVGFSYVILLESTVTGYLLHCRGWRSTYLYPKRPCFLGCAPTDFMEGMLQLVKWSSELFLLGISKYSPFTYGISRIPILHNFTFCYFTSTCQYIVALIVYGIIPQVCFLKGTPVFPKVTEPWFVVFAILYVSSQSQHLIEVLYGGGSLGTWWDEQRIWIVKSIVGGIFGSILAIKKRFGLNKAKFILSNKVVAKEKFEKYEQGKFEFEDAALFMSPLVGLLIVNILCFFGGLWRLFNVKDFEKMSGQLFLLGYLAALSYPIFEGIITMKSKVQ

SEQ ID NO: 101 - DNA, 2336 bp, Glycine max
  source 1..2336: mol_type = unassigned DNA
tatatgcatgttgaccggtaaacatggcgaccttccacacagaaaccgtgcaatcagggttggccttgagcagactccacatcctattccactcggtggcactcttgtttctctattactaccgcataagccacatcttactggaaccaagctttgtatggattttcatgaccatagcggagcttatcttcggcgagctctggctcttcaaacaggcgttccggtggcggcccgtgtcgagggccgtcatgccggagaagctgccgagcgacggcaagcttccggcgctcgacatcttcgtctgcacggttgaccccgaaaaggagccgacggtgcaggtgatggacaccgtcatctccgccattgccatggactacccctccaacaagctcgccgtgtacctttccgacgatggcgggtgtccggtgactctgtatgggatcagagaggcttctcggttcgcaaaggagtgggttccgttctgcagaaagtatgggatcaattcacggtgccccaaggccttcttctctcccatgggggaggatgaacgtgaactgcttcttcttcgcaaccatgaattcttggcagagcaagaacaactcaaggctaaatacaatataatgcaaaaaaatattgacgaatttggaagagaccctaaaaatcgttccattgtgtttgatagaccagctcgcattgagattataaatgagcaatccgaaataccactggttgtttatgtgtctcgtgaaagaaggccaaatgttcctcatacatacaaagggggagccctcaacacattgctcagagtctcagggctattcagtaacgggccctatgtacttgtagttgattgtgatatgtattgcaatgatccatcatcagctaaacaagccatgtgcttttttcttgatcctgaaacctccaaagatattgcttttgtccaattccctcaaatgtttcacaaccttagcatgaaagacatctacgatagtcaacataggcatgcttttacaacaatgtggcaaggaatggatggactaagaggtccaggtctttctggtagtggcaattacttaagtagaagtgcattaatctttccaagcccatatgaaaaagacggctatgaacataatgcccaaaacaaatttggcaactctaccatgtacattgaatcattaaaggccattcaaggacaacaaacttataaaacgagcatttcaagaaatgtgattttacaggaagcacaagcagtggcctcttgttcctatgaaatagacacaaattggggtaatgaggtaggattctcatatgttatattactggagagtacagttactggctatcttcttcactgtagaggatggagatcaacttacctttaccccaaaagaccttgtttcttgggatgtgcccccactgacttcatggaaggcatgcttcagttggtgaaatggagttctgaacttttcttgctaggaatatccaaatacagccctttcacttatgggatttcaagaattcctattctgcacaactttaccttttgctacttcacatctacatgtcaatatattgttgccttaatagtatatggcatcattcctcaagtatgcttcttgaaaggaactcctgtgtttcctaaggttacagaaccatggtttgtagtttttgcaatattatatgtatcctctcaaagtcaacatttgattgaagtcctttatggtggtggctctttgggaacatggtgggatgaacaaagaatatggattgtaaagtcaattgttggaggcatatttggatctatactagcaatcaagaaacgttttgggttaaacaaagcaaaattcattttatcaaataaagttgttgccaaagagaagtttgagaaatatgaacaaggtaagttcgagttcgaagatgcagctttgttcatgtctccattggttggattactcatagtgaatattctttgcttctttggtggtttatggagactatttaatgtgaaagattttgaaaagatgtctggccaactttttctacttggctatctggcggcgctcagttatcccatttttgaggggataataaccatgaaaagcaaggtgcaatagtagtttgtcaatgattaggctaatttaggtatttgaactttgttcacaaataatttgcttcatatgaaaatctaaagtgcatgctaaatgtttgtatcttaatatgtaattagcgtgcttttatttcatgcatgagaatatggctatcgattttaattaggagcaaaatgtatgttcttactccatttttaatgcaatttctttattttcttgccaattaaa

SEQ ID NO: 102 - protein, 695 aa, Glycyrrhiza uralensis
MASFTLHTETVQSWLLLSRLHILLHLAVVLLLLYYRITRFPFHAPTLPWTLMTVGEAIMAVLWFFNQAFRWRPVSRSVMTEKLPSDAKLPGLDIFVCTLDPEKEPTVEVMNTLVSALAMDYPPDKLSVYLSDDGAAPVTLYGVREASEFARVWVPFCKKYGIKSRCPKVFFSPSAEDEHLLRTDEFRSERDLIKAKYEKMQKNIEKFGSDAKNCRMVTDRPPRIEILIDQPDMPRVVYVSRERRPSLPHKFKGGALNTLLRVSGLISNGPYVLVVDCDMYCNDPSSAKQAMCFFLDPETSKSIAFVQFPQMFHNLGKKDIYDNQSRTAFKTMWQGMDGLRGPGLSGSGNYLNRSALLFGSPNQKDDYLDDAQNYLGKSTMYIESLKAIRGQKTMKKNISRDEILREAQVLASCSYETNTEWGAEVGFSYGILLESSITGYLFHCRGWKSAYLYPKTPCFLGCAPTDIKEGMLQLVKWLSEYCLLGFSKYSPFTYGFSRMPIMPTLVYCFLTTTTLYSIVFILYGIVPQVCFLKGIPVFPKVTDPWFAVFATLYISTQIQHLIEVLSGDGSVAMWWDEQGIWILKSVTSVFAIIEAAKKGLGLNKKKFMLSNKAIDKEKLKKYEQGRFDFQGAALFMSPMVVLLIVNVVSFIGGIWRLFNAKDIEDMFGQLFLVSYVMALSYPIFEGIITMKSKSG

SEQ ID NO: 103 - DNA, 2088 bp, Glycyrrhiza uralensis
  source 1..2088: mol_type = unassigned DNA
atggcaagcttcacccttcacacagaaaccgttcagtcatggctactcctcagcagacttcacatactgctgcacctcgcagttgtactgctcctcttatactaccgcatcacacgtttccccttccatgctccgactctaccgtggactctgatgaccgtaggtgaggctattatggcagtgctgtggttcttcaaccaggccttccggtggcggccggtgagccgctcggtgatgacggagaagctgcccagcgacgcgaagctgccggggcttgacatattcgtgtgcacgcttgaccccgagaaggagcccaccgtggaggtgatgaacactctggtctctgcccttgccatggactacccccctgacaagctctccgtttacctctccgacgatggcgccgccccggtcactctttacggcgtgagagaggcttctgagttcgcgagggtgtgggtccctttctgcaaaaagtatgggatcaagtcaaggtgtcccaaggttttcttctctcccagtgctgaggatgaacaccttcttcgcaccgacgagttcaggtcagagcgagacctcatcaaggctaaatacgagaaaatgcagaaaaatattgagaaatttggttcggatgccaaaaattgtcgtatggtgactgacagacctcctcggatcgagatattgattgaccaaccagacatgccacgtgttgtttacgtgtctcgggaaagaaggccatcactccctcacaagttcaaaggaggagccctcaatacattgctcagagtctcaggtctaatcagcaatgggccttatgtacttgtagtggactgtgatatgtattgcaatgacccatcctcagccaaacaagccatgtgtttctttcttgatcctgaaacctctaaatctattgcatttgtccaattccctcaaatgtttcacaaccttggcaaaaaagacatctatgacaatcaatctaggactgcttttaagacaatgtggcaagggatggatggactaagaggtcctggtctttctggcagcggtaattacttgaatagaagtgcattactatttggaagtccaaatcaaaaagatgactatctggatgatgcccaaaactacttaggcaagtctaccatgtacatagaatcactaaaggccattcgtggacaaaaaactatgaaaaagaatatttcaagagatgaaattttacgagaagctcaagtattagcctcttgttcctatgagacaaacacagaatggggagcagaggtaggattctcatatggcatcttactggagagttcaatcactggctatcttttccactgcagaggatggaaatcagcatatctttacccaaagacaccatgtttcttagggtgtgccccaactgacatcaaggaaggaatgctccaattggtgaagtggttgtctgaatactgcttgctaggattctctaaatacagccctttcacttatggcttttcaagaatgcccattatgcctaccttagtctattgcttcttgacaacaacaaccctttattccattgtcttcatcctttatggcattgtcccccaagtttgcttcttaaaaggaatacccgtgtttccaaaggtcacagacccttggtttgcagtgtttgcaacactgtatatatccacccagattcaacatttgatagaggtcctttctggtgatggctctgtggcaatgtggtgggatgaacagggaatctggattctgaagtcagtcactagcgtgttcgcaatcatagaggcagctaagaaagggttaggattgaacaagaagaaattcatgttgtcaaacaaagcaattgacaaggagaagctcaagaagtatgagcaaggtaggtttgatttccaaggtgcagctctgttcatgtccccaatggttgtgttgctcatagtgaacgttgtttccttcattggtggcatatggagactattcaatgcaaaggatattgaagatatgtttggtcagcttttcctagttagttatgtaatggcccttagttatcccatttttgaagggataataaccatgaaaagcaagagtggatag

SEQ ID NO: 104 - protein, 694 aa, unidentified
  source 1..694: note = Lotus japonicus
MANFTLHTETVQAWLPLSRLHILIHSVFVILLLYYRTTRLIHAPTAPWILMTVAEALLAVLWLFNQAFRWRPVSRSVKTEKLPRDENLPGLDIFVCTIDPEKEPTAGVMDTVVSAVAMDYPPDKLSVYLSDDGGCAVTEYGIREACEFAKVWVPFCRKYGIKSRCPKVFFSPMGEDEEILRTDEFRAEQEKIKAQYETMQKNIEKFGSDPKNCRIVTDRPSKIEVINEQSEIPRVVYVSRERRPSLPHKFKGGALNTLVRVSGLISNGPYVLAVDCDMYCNDPSSAKQAMCFFLDPETSKYIAFVQFPQMFHNLSKKDIYDNQSRTAFKAMWQGMDGLSGPGLSGSGNYLSRSALLFGSPNQKGDYLLDAQNYFGESPLYIESLKAIRGQQTTKKNISRDESLLEAKVVASASYETNTEWGSEVGFSYGILLESTITGYLLHCRGWKSAYLYPKTPCFLGCAPTDIKEGMLQLVKWLSELCLFAVSKYSPFTYGFSRLPIMPTFTYCFLAASSLYAIVFILYGIVPQVCFLKGIPVFPKATDPWFAVFAVLYVATQIQHLIEVLSGNGSVSMWWDEQRIWILKSVTSVFAMIEGIKKWLGLNKKKFNLSNKAVDKEKVKKYEQGRFDFQGAALYMSPMVVLLLVNIVCFFGGLWRLFKEKDFADMFGQLFLLSYVMALSYPILEGIVTMKMKSG

SEQ ID NO: 105 - DNA, 2085 bp, unidentified
  source 1..2085: mol_type = unassigned DNA; note = Lotus japonicus
atggccaatttcactctccacacagaaaccgttcaagcatggctccctctaagcagactccacattcttatacactcagtgttcgtcatccttctcctctactaccgcacaacgcgtctcatccacgcgccgaccgcgccgtggatcctgatgaccgttgcggaggctctcctcgccgtgctttggctcttcaaccaggccttccggtggcgaccggtgagccgctccgtgaagacagagaagctgccgcgcgacgagaatctccccgggctggacatatttgtgtgcacgattgatcctgagaaggagccaacggcaggggtgatggacacggttgtttccgccgtggcgatggattacccgccggataagctatccgtgtatctttctgatgatggtggttgcgccgtgacggagtatgggattagagaggcttgtgagtttgccaaggtgtgggttcctttttgtagaaagtatgggatcaagtcgaggtgtccaaaagttttcttctctccgatgggggaagatgaagagattctaaggacagatgagttcagagcagagcaagagaagatcaaggcccaatacgagactatgcagaaaaacatcgagaaatttggttcagaccccaaaaattgtcgtattgtgactgacagaccctctaagatcgaggttataaatgagcaatcagaaatcccacgtgttgtgtacgtctctcgtgaaagaaggccatcacttcctcacaagttcaaaggaggagctctcaacacattggtcagagtgtcaggtctaatcagcaatggaccttatgtgcttgcagtggattgtgatatgtattgcaatgatccatcctctgccaagcaagcaatgtgcttcttccttgatccagaaacatctaaatacattgcatttgtccaattccctcaaatgtttcacaaccttagtaagaaagacatctatgataatcaatctaggactgcttttaaggcaatgtggcaaggcatggatggactcagtggtccaggtctttctggcagtggtaactacttgagtagaagtgcattgctatttggaagtccaaaccaaaaaggtgactatctgcttgatgctcaaaactactttggcgagtctcccttgtacattgaatcattgaaggccatccgtggacaacaaactaccaaaaagaatatctcaagagacgaaagtttactagaagctaaagtggtggcctctgcttcctacgagacaaacacagaatggggctcagaggttggattctcatatggcatcttactggagagtactattactggttaccttttgcactgcagaggatggaaatcagcttatctttacccaaaaacaccatgtttcttagggtgtgcccccactgacattaaagaaggcatgcttcagttggtgaagtggttgtctgagctttgcttgtttgctgtctctaagtacagcccttttacatatgggttttcaagattgcccattatgcctaccttcacttattgtttcctggcagcttcatccctatatgctattgtcttcatcctttatggcattgtacctcaagtgtgcttcttgaaaggaatccctgtgtttccaaaggccacagacccttggtttgcagtgtttgcagtattgtatgtagccacccagattcaacatttgattgaagtcctttctggcaatggctcggtctcgatgtggtgggatgaacaaagaatttggattctgaagtcagttactagcgtatttgcaatgatagagggaatcaagaaatggttaggattgaacaagaaaaaattcaacctgtcaaacaaagcggttgacaaggagaaggtcaagaaatatgagcaaggtaggtttgatttccaaggagcagctctgtacatgtctccaatggttgtgttgctcctagtgaacattgtttgcttctttggcggtttatggagactgtttaaggagaaagattttgcagatatgtttggtcaacttttcctactcagctatgtgatggctctcagttatcccattcttgaggggatagtaactatgaaaatgaagagtgggtag

SEQ ID NO: 106 - DNA, 350 bp, synthetic construct
  misc_feature 1..350: note = SOAP5_VIGS_spinach
  source 1..350: mol_type = other DNA
ttccgtagccatcttctcgctcttctactaccgtttcacttccttcttcaactccgacatctccatacttgcttactccttactcaccaccgccgaactcttcttaacctttctatgggcttttactcaggctttccggtggcgtcccgtaatgagggaagtctccgggtacgaatccatcaaacccgaacaactaccgggtttggatgtcttcattgtcactgctgacccgacaaaggagccagttctggaggtgatgaactccgtgatatcatccatggcgttggattatccggttgatagactggcggtttacttgtcggatgacggtggttctccgttgtcgaagg

SEQ ID NO: 107 - DNA, 438 bp, synthetic construct
  misc_feature 1..438: note = BvCSL_VIGS_red beet
  source 1..438: mol_type = other DNA
ccttcggctcctccacaaaattcattgcgtcagttagttcaaactccaagcaaaatatggccttgaaggaaatgacaagagacgacttgttagaagaggctaaaaatttggctacttgtgcatatgaatcaaacactgaatggggtaacaagattggatattcgtatgagtgtttgttggagagtacatttaccggatatctcttacattgcaaaggatggatttccgtgtatctttacccaaaaagaccctgcttcttaggatgcacgacgattgacatgaaagatgccatggttcaactaatgaaatggacctctggattactaggagttggcatatcaaagtttagccctctaacttatgccttttcgaggatgtctatattacaaagcatgtgctacggttacttcacattttcagcccttttcggagtttcgt

SEQ ID NO: 108 - DNA, 2148 bp, synthetic construct
  misc_feature 1..2148: note = MsCSL_asRNA_Medicago sativa
  source 1..2148: mol_type = other DNA
ctacccactcttccgcttcatagatataatcccctccacaataggatgacttagagcaagtatatagataatgaggaaaagttgaccaaacatttcatcaaaatccctcacattgagtagtctccataaaccaccaaagaagcaaacaatatttattgtgagtaacacaatcactggtgccatgagcaatgctgcaccatcaaaattaaacttaccctgctcatatttctttagcttatctttgtcaattgctttgtccgataaagtgaattttgccttgttcaacccaaacttttttttcgctgtttctataattgcgaaaacgcacccaattgattttacaatcccacttctttgttcattccaccaaatcatcgaggagccaccagttgaaattacctcaatataatgatgaatttgactggatagatacaatactgtaaacactataaaccaaggatctgtaacctttggatatacaggtattcccttcaagaagcaaatctgaggaacgaatccatataagatgtaggctgttgcaaattgggttgtgcttaccaagtaacaaaaagttaaacaatgaatagtgggcaatcttgaaaggccataagtgaatgggctatatttagagattgcaagcaacaaaagttcagatgaccactttatcggttgaatcaatccctctttcatgtcagttggagcacatcctaagaaacatggtcttttagggtaaagataagttgatctccatcctctacaatggaggagatagccagtaacggtactctcaagttttatagcatacgagaatcccacctcattaccccaatttgtgtttctctcataggaacaagaagccacttcacatgcttcttgtaaaattacatctcttgaaatattctgcttcttagtttgttgaccacgtaacgcttttagtgattctacatacatgttagacttgccaaagttgtatagagcatcaagtagatagtcccctttttggtttggacttccaaagagtaatgcacttctacttatataatttccactgccagttagacctggacctcttaatccatccattcccttccactttgtcgtaaaagcagtcctagtctgactatcatatatgtctttcttgctaaggttgtgaaacatttgagggaattgaacaaaagcaacatctttagaggtttcaggatcaagaaaaaagcacatggattgtttggctgatgatgcatcattacaattcatatcacaatctacgacaagtacataaggtccattgctgattagccctgacaccctaagcaatgtattgagagctcctcctttgaatctgtgaggaacagatggtcttttttcacgagatacataaacaactagtggcatttctttttggtcattaatgatctcgatccgagaaggtctatcggtcaccatacaaagattctttaggttgcttccgaatttctcaatatttttctgcattttctcgtatttggctttggtgggcatatgttccttggagttttcaacttgtgatgagttaggatccactctatttaatctcttcttaatctggtccctcacttcttcaaattcacgaggtcgatgaagtcgttcattctcacccaaagcagtgaagaaaaacttaggacaccttgacttaacatcatattttttacaaaaaggaacccaaactttagcaaattcaaaagcctctttgatcccaaaaagagtaataggagaacctccatcatcagaaagataaatagaaagtttattagaagggtaatccattgcaatagcagaaataacagtgttcataacatcaaccgttggttctttttcaggatcaatggtacacacaaatatgtcgagtcccggcaacttctcctccggcggtaatttctcggtcataactgaacgattcaccagcctccaacggaatgcttggttgaaaaaccatagaaatgatagaataatctcagctattgtcattagaaaccatggatatgaaataaacaaattgctgatacggtagtaaaagagaaacaagacacatgtgaagtggaagattatgtaagctcttcttagaggtaacaatggttgaactgtttctttgtgaaatgtgaaggttgccat

SEQ ID NO: 109 - DNA, 1431 bp, Spinacia oleracea
  source 1..1431: mol_type = unassigned DNA
atggccaaatctgagcaagaaacaatggccaaatctgagcaagaaacatccatcaaacttgtttcagaatgctttgtaaaaccaaaatatgagattaaatccgctaagcaaccttaccacttaggtcccatggatctagttatgttaactatcgatcctatacaaaaaggtcttgtctttacaataaagaattccccactctttttgtcatccgaatcccatgataatattgaaattattcgaaccaaagttgtgtcacgtttattagaaaagcttaaacactcactttctatagctctagtccacttctacccgttagcaggtcgtttcactacacaaaaacaacccgagcataacacgagcttggtctttattgattgcaacaaaggtcccggagcgcggttcatccacgctacttcccttgactttactatctccgatatactttcaccggttgatgtttccatcgttcattctttctttgatctcggtgaaaagcatgtaaactacgattgtcatactaaggcgttgctatcgatccaggtaacagaacttttagatggggtgtttattgggtttagcatgagtcatagtgtggttgatggtacctcttttattcattttgtcaataccttgtctgaaatttttaaatctgatgattttaccactatttcacgtgccccaatacttaattataggccttgtgatattccgatccttaaatttccgtttcttgatgtggaggggtttatatgtcgtgcgtataaccctgggccgttaagggaaagaatcttccacttttcactaaattcgatgctgagactcaaggccatggctaaccaagaatgtggtacccaaaatgttttatcatctttccaagctttgactgcggttgtatggaggtccatcacccgagttcggaacttaccaaaggatgagcaaaccacgtgttttatggctatgggttctcgaactaggctcaacccgcctctttcggatgactattttgggaattttatgattagtaccaaatttgcttgcaaggcagaggaattattgggtaacagtttaggttgggtagcaatgaatttacgtaaaatcattatgtccactgacgagaaatcgatacttgctacgtacaaagcattggctgattccccaatagtgattccgcgtgaaacgatccccggtcctcatgggatgaccagagtaataattggaggatcttcaaggttcgatatgtatgggcctgaatttggattgggtcgagctttggccgctcgcatgggttatgggaataaggatgatgggaaaataacagcaaatcctgggtgtgaaggaggtggaagtgttgatttggaaatttgccttaggcctcatattatggcctctcttgaagttgatcaagagtttatgggttttgtgtcctag

SEQ ID NO: 110 - DNA, 1191 bp, Spinacia oleracea
  source 1..1191: mol_type = unassigned DNA
atgactccaaatctgcaaatagtaaccaacggaggcaaaccggaaaatgatgaagcagaacccgtatcacctaccggacaatacttcaacagcaaagtgttgtctgtttgtgtccttgccattctagaaattgatgttcctatagatgactcgtgtgtaattccacaactccgtgatgtcttcctacccatgaaccccagattttcatctatcatgatatctgacaataaagatgtaaaacaatggaaaagagtggaagtgaaccttcaagatcatgttgtcgtccctagcgtcccagatggcttatcggttgaatcatacgacaagtactttgatgaatatctgacaaaaataacagtggatccattaccacaggataggcctttatgggaacttcatgttataaaatacccaacaagcaaagcagcgggtcatttcatctggaagcttcaccatgcacttggtgacggctacactctaatgggagtacttctgtccggcgtgaacagagcagatgatccttcccttccgttaactttcccttcaacacgatcaagctcactagttacaaacaacaagatgaatattatcagctgggtgccaagaactttttcagcaatctacaacggtgtttataattttggatggagttttctaaaaagcacttgcaaggcagatgataagacacctatcagatccggaaatgaaggtctgggtttccacccaatgaagatctcgacaatagaactatccctagaccaaatcaaatttatcaaaacaaaactcggcgcaacggtaaatgacattcttgcaggcataattttcctcggggttcgaaaatacatgcaagcaactgatacagaatctggaaactcagaatcaacggcattggtgctgtttaacactaggaacattggaggttatatgaccgctgagcaaatgaagaaagcacaaatgaaaatatgggggaaccaatttgcatttttgcatatagcaatacctcaattaatcaatgacaaatgctcgaacccccttgactatgtctatgaagcacgaaaacagatctctaggttcaaaagctcaccatcagtctatctaacagctcagtgcctagagctgctaggaaatgcaaaggacctgaggcagcagctgaatttatccagagtacaacgaataaagcaagcatattaa

SEQ ID NO: 111 - DNA, 1043 bp, Spinacia oleracea
  source 1..1043: mol_type = unassigned DNA
atgttagagctagcagaagacgaggtgaagaacttcttcaaggtatgggcaatagtttttgcatctttaagctattgttattacataggcaagctaattaatccaaaaggttatacaagattagtagcaataatcccaattattactctctttttagcacttcctttaaatctcacatcttttcatcttggtggtatgacttgtttctttattgcttggcttgctaatttcaaactcttgctttttgcttttgataagggcccactttgcgctaattcttcaatctcattcgccaaatttcttgcactttcttgcttacccatcaaaatccaacacccacctcataaaaagtcattaaaatcacacccatctatttataattacatcattaaagggatacttttatgtctaataattaaaatctatgattatggtgattacattcatccaaaaatcatatggctaatctttttcttccactcctattttaccatagagttagtctttgcattcctagcaacatcgactaatattttgttagggctcgaactggagccacagttcaatgaacccttaatatcaacctcattgcaagacttttggggtaagagatggaatatcatggtgacaaggatacttaggcctacggtgtaccttcccacactagagtactccactaaggtcgttggacgcacgtgggccacacttccggcggtgatgtccacgttctttgtgtcagccattatgcacgagctcatcttctactacttggggcgcaactggcccacattcgaggtgacgtggttctttctcctgcatggattatgtctttgtgttgagattgtcgctaagaagttagttggtgggaaatggaggatcccacggtggatttccggccctgccacggtgttgtttgtggtgggtactgggttttggctgttcttgccgccgttgttgaaggctgggttggatactagaccgtttcaagagtttgcggccgttgccaagtttgtaaggagtttgaaggcagctctcacattttg

SEQ ID NO: 112 - DNA, 1530 bp, Spinacia oleracea
  source 1..1530: mol_type = unassigned DNA
atggctcctccttcttcttcttcaactacgggttctggtaatggttctagttttgcagtcaatataatggcgtcgttctacatttccccacaacaaccttccaccacaaattcacattctatccctctcactttctttgacattccttggcttcaatatcctccgctccaacctctcttcttctttcaacttccatctacaccccaatcttcttcttcttcttcttcttcttcttcttcttctttcgaccacaacttgtacttggagtttagctccaccatcctccctaggctcaaacactccctcgcttctgccttgcaatattactttcccttttctggaaaactcaccactactacccatactatcccgaataacctagttttctcgacagactcatcagattctgttgagttgactgtttctctgtgtgatgctgattttaatggtctatgcagctttctacccaggtctactcatctcttccaacaattggttccctccttgccaaatattgaatcctccaacctcactacattccctgcacctttattagctattcagatcacattctttcccacctcttctcctggtttctctattggctttgcttctcatcctgtgctttctgatcagaggaccttcagtaacttcctttactcttgggcctctttctccaagtttgataatctaaacatttcacttgccccttccttccctgtctctgacaggtctgtcattctcgaccctgatagacttgagccccttctgttggagcagtggttgggattggagtccaaaccaaccatgtcaacaaagatgaagctacgtcctcctcctgcttatgtccgtggctcgctccggtccacattcgtcatgggcccatctgatattgctaatgctacacaatggttacaaacccagtgtgagaagctcaacagatcatatcctgttctcttgtcaccctacgtcgtcacttgtgcctttatatggacctgttttctgagagcccgagtccagaacagtgctgttactaaagccaaagcaaaaggcaccatgtactttggatttattgctggtggtattacccgtttaccctatcgggtacctgctaagtatcttggcaactgtgtcgggtttggacgggcagcagcgcagagggaggagctactgaaggaaggtgaggggatgttggcagctgctgatgcaattgggctaaccattaaaaagttggataaagatgttttaggaggagctgagaaatggatatatgaatggcagacattaatggaatccgaagatcatattcatgtggttgggtcgcccaaggtgaacctttatgagacggatttttggtgggggaaaccgaagaagatagaggaaatttcaactgatgttaccagagccatctctcttacacagagcagggacatgaaaaggggaattgaaattggcctcactttaccaaactccattatggatgacttctcctctatcttcactcaaggcctccttgtttttcaaaattag

SEQ ID NO: 113 - protein, 476 aa, Spinacia oleracea
MAKSEQETMAKSEQETSIKLVSECFVKPKYEIKSAKQPYHLGPMDLVMLTIDPIQKGLVFTIKNSPLFLSSESHDNIEIIRTKVVSRLLEKLKHSLSIALVHFYPLAGRFTTQKQPEHNTSLVFIDCNKGPGARFIHATSLDFTISDILSPVDVSIVHSFFDLGEKHVNYDCHTKALLSIQVTELLDGVFIGFSMSHSVVDGTSFIHFVNTLSEIFKSDDFTTISRAPILNYRPCDIPILKFPFLDVEGFICRAYNPGPLRERIFHFSLNSMLRLKAMANQECGTQNVLSSFQALTAVVWRSITRVRNLPKDEQTTCFMAMGSRTRLNPPLSDDYFGNFMISTKFACKAEELLGNSLGWVAMNLRKIIMSTDEKSILATYKALADSPIVIPRETIPGPHGMTRVIIGGSSRFDMYGPEFGLGRALAARMGYGNKDDGKITANPGCEGGGSVDLEICLRPHIMASLEVDQEFMGFVS

SEQ ID NO: 114 - protein, 396 aa, Spinacia oleracea
MTPNLQIVTNGGKPENDEAEPVSPTGQYFNSKVLSVCVLAILEIDVPIDDSCVIPQLRDVFLPMNPRFSSIMISDNKDVKQWKRVEVNLQDHVVVPSVPDGLSVESYDKYFDEYLTKITVDPLPQDRPLWELHVIKYPTSKAAGHFIWKLHHALGDGYTLMGVLLSGVNRADDPSLPLTFPSTRSSSLVTNNKMNIISWVPRTFSAIYNGVYNFGWSFLKSTCKADDKTPIRSGNEGLGFHPMKISTIELSLDQIKFIKTKLGATVNDILAGIIFLGVRKYMQATDTESGNSESTALVLFNTRNIGGYMTAEQMKKAQMKIWGNQFAFLHIAIPQLINDKCSNPLDYVYEARKQISRFKSSPSVYLTAQCLELLGNAKDLRQQLNLSRVQRIKQAY

SEQ ID NO: 115 - protein, 347 aa, Spinacia oleracea
MLELAEDEVKNFFKVWAIVFASLSYCYYIGKLINPKGYTRLVAIIPIITLFLALPLNLTSFHLGGMTCFFIAWLANFKLLLFAFDKGPLCANSSISFAKFLALSCLPIKIQHPPHKKSLKSHPSIYNYIIKGILLCLIIKIYDYGDYIHPKIIWLIFFFHSYFTIELVFAFLATSTNILLGLELEPQFNEPLISTSLQDFWGKRWNIMVTRILRPTVYLPTLEYSTKVVGRTWATLPAVMSTFFVSAIMHELIFYYLGRNWPTFEVTWFFLLHGLCLCVEIVAKKLVGGKWRIPRWISGPATVLFVVGTGFWLFLPPLLKAGLDTRPFQEFAAVAKFVRSLKAALTF

SEQ ID NO: 116 - protein, 509 aa, Spinacia oleracea
MAPPSSSSTTGSGNGSSFAVNIMASFYISPQQPSTTNSHSIPLTFFDIPWLQYPPLQPLFFFQLPSTPQSSSSSSSSSSSSFDHNLYLEFSSTILPRLKHSLASALQYYFPFSGKLTTTTHTIPNNLVFSTDSSDSVELTVSLCDADFNGLCSFLPRSTHLFQQLVPSLPNIESSNLTTFPAPLLAIQITFFPTSSPGFSIGFASHPVLSDQRTFSNFLYSWASFSKFDNLNISLAPSFPVSDRSVILDPDRLEPLLLEQWLGLESKPTMSTKMKLRPPPAYVRGSLRSTFVMGPSDIANATQWLQTQCEKLNRSYPVLLSPYVVTCAFIWTCFLRARVQNSAVTKAKAKGTMYFGFIAGGITRLPYRVPAKYLGNCVGFGRAAAQREELLKEGEGMLAAADAIGLTIKKLDKDVLGGAEKWIYEWQTLMESEDHIHVVGSPKVNLYETDFWWGKPKKIEEISTDVTRAISLTQSRDMKRGIEIGLTLPNSIMDDFSSIFTQGLLVFQN

SEQ ID NO: 117 - DNA, 1419 bp, Spinacia oleracea
  source 1..1419: mol_type = unassigned DNA
atggccgcctcaaacaaagagcaaagcaagctacatattgctatgtttccttggtttgcatatggtcatataaacccattcatccagctctctaacaagctttcctcccatggcatccaaatctctttcttctcaataccaggaaacattgatcgtatcaaatcctcccttaatctctcacctcccaaccagctcatccccctcactattcccccaactgaaggactctctcccaactttgacagcagctctgaagtaacacctcaaactgctcagcttctcacactagcacttgatcaaatgcagccccaagtcaaagctctattccctcacccccagccacaagttatcctctttgattttgcatatcactggcttccctcagtagcttctgaactaggcatcaaagctgttcatttcaatacattcccagctgttatcaattcatatctcactgtcccttcaagaatgactgatccaaataaaccaccgacatttgaggacttgaagaaccctcctcaaggctatcccaaaacctcaaccgcctcagtgaaaaccttcgaagctcaagattacctattccttttcaagagtttcgatggcggaccgtgccatttcgaaaagatattggcattcacaaacagctgtgatgctatactttacaggacctgcaatgaaatagaaggtccattcatagattacttcaagacccaaataaataaaccactgcttttagctggcccaagtgttcctctaccaccctctggtgaactggatgaaaaatgggagatgtggttaggtaaatttcctgaaaagtcagtcatatactgcagcttcggaagcgagacatacttgaatgatgctcagattcaggagcttacacttgggttggagctcactggtctgccctttatcttggttttgaattttggaacaagtaacagcaccgatgcccacaataagctagaagcatcattaccagaaggatttagagagagaatcaaagacaggggcgttctgcatacaggatgggtgcaacagcaaaacattttagcacacagaagcataggatgctttcttactcacgcagggttcagctctgtaatagagggtattgtgaatgactgtcaattagcatttctacctctaaaggctgaccagtttatgatcgctaagctatttagtggggatctgaaagcaggggtggaggtaaatcgaagagatgaagatgggtcttttgccaaagaagatattttcgaagcaataaagacgatcatggtggatactgataaagaaccaagtagatccatcagggagaatcatagcaattggagaaagtttttgatgaataaggagattgaagctagttatattgcaaatttagctcatgaactcaaggcattggttcaaaaagcttag

SEQ ID NO: 118 - protein, 472 aa, Spinacia oleracea
MAASNKEQSKLHIAMFPWFAYGHINPFIQLSNKLSSHGIQISFFSIPGNIDRIKSSLNLSPPNQLIPLTIPPTEGLSPNFDSSSEVTPQTAQLLTLALDQMQPQVKALFPHPQPQVILFDFAYHWLPSVASELGIKAVHFNTFPAVINSYLTVPSRMTDPNKPPTFEDLKNPPQGYPKTSTASVKTFEAQDYLFLFKSFDGGPCHFEKILAFTNSCDAILYRTCNEIEGPFIDYFKTQINKPLLLAGPSVPLPPSGELDEKWEMWLGKFPEKSVIYCSFGSETYLNDAQIQELTLGLELTGLPFILVLNFGTSNSTDAHNKLEASLPEGFRERIKDRGVLHTGWVQQQNILAHRSIGCFLTHAGFSSVIEGIVNDCQLAFLPLKADQFMIAKLFSGDLKAGVEVNRRDEDGSFAKEDIFEAIKTIMVDTDKEPSRSIRENHSNWRKFLMNKEIEASYIANLAHELKALVQKA

SEQ ID NO: 119 - DNA, 1386 bp, Spinacia oleracea
  source 1..1386: mol_type = unassigned DNA
atgtgtgacgacaaaaaatcatctgttttgagcatagcattttatccgtggtttgctcttggtcaccttacttcatttctccgattagccaacaaacttgcacaaaatggtcacaatgtgtcctattttatcccaactaatacattacctagattacttcctcacaaccattaccctggccaccttactttcatccccgtcaccgtcccacccgttgacggcctccctctcagagccgagaccaccaacgatgtcccctcctcggctatacaccttcttatgactgccatggatttgacccgtgacactatcgaggcccatttggttagtatcaaacccgatgttgttttctacgactttgcttattggattcccgatctagcccgaaaacacgggttcaagtcagtactctacattacatcctatatagcaagatgtgcttattttgcccccgatttgaagtcgggtcatcagtccactggggccgaaattattgcgccaccaccgggttttccgtctcagcatttccggatgcaagcacacgaggctgagactgtggcagacgtaggtaaagagcaagatggattacaaggtataactatttctgaaaggatgcgcattgcttttggaaaatgcgacgcaattggagtaaagagttgtaaggagatggaaaaggtgtatattgactactgtgagaagatatttggtaagtctgtactactagcaggtcctatggtccctaaaaccccatcttccaaacttgatgaatattttgatggttggcttacgggttttggtgctgctactgtgatttattgtgcatttgggagtgaatgtgttctcgaaattaaccaatttcaacaacttcttcttggactagagctcacaggaaggccatttttggtggccatgaagccgcctaagaagtatgaaacaatagagtcggccttaccagaagggtttgagaagagaacaaaaggaaggggaatcgtacatgagggttgggtgcagcaacaactgatattgcaacatccatcagtaggatgtttcataactcattgtggagttgggtctctttcggaagctatggtcagcaaatgtcaagtagtgttgatgcctcaagctgtagaccaattcatcaatgcgaggatgatgagtttagagttgaagattggggttgaggttgagaagagagaagatgatggtttgttcacaaaggaggctgtgcataaggcggtctctttggtgatggaggaagaaagtgaagtcgcaaaagagatgagggtaagtcatgataaatggagagaattcttattacaggaaggtcttgaggattcttatatcagtagcttcattcagagtctacgacagttaacgattggatga

SEQ ID NO: 120 - protein, 461 aa, Spinacia oleracea
MCDDKKSSVLSIAFYPWFALGHLTSFLRLANKLAQNGHNVSYFIPTNTLPRLLPHNHYPGHLTFIPVTVPPVDGLPLRAETTNDVPSSAIHLLMTAMDLTRDTIEAHLVSIKPDVVFYDFAYWIPDLARKHGFKSVLYITSYIARCAYFAPDLKSGHQSTGAEIIAPPPGFPSQHFRMQAHEAETVADVGKEQDGLQGITISERMRIAFGKCDAIGVKSCKEMEKVYIDYCEKIFGKSVLLAGPMVPKTPSSKLDEYFDGWLTGFGAATVIYCAFGSECVLEINQFQQLLLGLELTGRPFLVAMKPPKKYETIESALPEGFEKRTKGRGIVHEGWVQQQLILQHPSVGCFITHCGVGSLSEAMVSKCQVVLMPQAVDQFINARMMSLELKIGVEVEKREDDGLFTKEAVHKAVSLVMEEESEVAKEMRVSHDKWREFLLQEGLEDSYISSFIQSLRQLTIG

SEQ ID NO: 121 - DNA, 1338 bp, Spinacia oleracea
  source 1..1338: mol_type = unassigned DNA
atggggtggccggtggtgggcgagtcatttgaattttttcaaaccgggtggaaaggttacccggaaaagttcatatttgatagactgaacaagtacaccccaagccaagtgttcaagacttccatcgtaggagaaaaggttgcggttttatgtggcgcggcgggtaacaagttcttgtactcaaacgagaacaagttagtacaagcttggtggcctagctctgttgataagatctttccttcttctacccaaacttcctccaaagaagaggctaagaagatgcggaaactcctccctaacttcctcaagcccgaggctttacataggtacatacccatcatggatagcattgccatccggcacatggagtccgggtgggagggaaaggacaaggtagaagtcttccctttggctaagaattacaccttctggctggcttgccgactcttcttaagcgtcgaggacccggctcatgtagccaagttctccgaaccattcaacgacatagccgcagggatcatctcgatgccaatcgacctccccggaacacccttcaaccgagggatcaagtcgtctaacgtcgtaaggaaagagttgagggccatcataaagcagaggaaacttgacttagcagatggcaaggcttcacctacacaagatattctgtctcatatgttgttgacttgtactgaagatggcaagtttatgagtgaaatggatattgctgataagattctgggacttcttattggtggacatgatactgctagtgcttcttgtacttttgttgttaagtttcttgctgagcttcctcacatatatgaaggtgtctacaaagagcaaatggagatagcaaattcaaaaaaagcaggagaacttctaaattgggaggacatacaaaaaatgaaatactcatggaatgtagcttgtgaagttatgcgtttggctcctccacttcaaggtggtttcagggaagccctttctgatttcatgtataacggattccaaatccccaagggctggaagttatattggagtgcaaattcaacacatatgaacccggaatgcttcccggagcccaagacgttcgacccatcgaggttcgacggtacgggaccagcaccatacacatacgtccccttcggaggaggaccgagaatgtgcccgggcaaggagtatgcaaggctagagatattagtgttcatgcacaacgttgtcaagaggtttaaatgggaaaaaatgcttcctgatgagaaggttattgtcaatcccatgcctatcccagaacatggccttcctgtccgccttttccctcatcctcgaactgtagctgcttaa

SEQ ID NO: 122 - DNA, 1077 bp, Spinacia oleracea
  source 1..1077: mol_type = unassigned DNA
atggaccgcactcggcctgaggtggagtctatcctatcaacccagaagcccgaccttgtcttctacgacatggcccattggatacccgatattgctagcaagctcgggatcaagtcagtgtcatacaacagtgtatgtgctatttctatgtcccatgtcagacctagcgtccttccaaaagcagcagcaacatcacaagtagctttgtcatcatcgtcatatgtcggtagtgtccctcagtggagtcgcaatgatcacaactcatcagtctattttggggatgagattaccttacttgaacgctctatcatctccctctcatctgcagatgcaatagccatccgcacatgcagggagattgaaggagtatattgtgaccgtgttgctgccacattcaacaagcctgtccttgtcactagccacgccttgcctgatcttgaactccaactctctccgttggagactcgatgggctgactggctagctaggtttgggccaagatcagtgatcttttgctgccttggtagtcagcatgtcttagacgcagcccaactgcaggagttagccttggggttagaaatgacaggactacccttcttgatggctgtaaagcctcctgtaggctatgcctcattgaaggagctgctgccagaaggttataatgatcgggttagtgggagaggggtggttcatggtgggtgggtgcagcaacagcagatactggcgcacccatcattagggtgctttgtgacccattgtgggtcttcgtccatgtgggaggggttagtgagtgaaactcagctggtactactcccacagctgccagaccaaactctgaacgcaaagttgatggcagatgagctcaaggtgggtgtgaaggtggagcgacaacagaacgggtgggtgtcaaagcaaggtctatgtgaagccatcaacagagtgatggatgaagatagtgatataagtcatgtagtgagaaagaatcatgctaaatacacaagtatgttgattagccctgactttatcactggctacattgacaacttcatcaaggatttacaagcccttatttcttag

SEQ ID NO: 123 - protein, 366 aa, Spinacia oleracea
MLVYDAYFPWALDVAKDHNILAAAYFVSSCAYVASFYPMFLEEFGSDDQHPVVAAESVPGLLVELPSREEMERYAPKCAQSSSSDDKPDTGKKPLHPVYQMVVSSITTLHLADFVLFNSFDHLEHQVVKWMTNLWRVKTVGPLLPSAYLDKRIESDVNYCVKPYKPNNEACMSWLNAKQVASVVYVSFGSVAKLSVEQISEIAKALKQIPSSFLWIVREAEQEKLPNDFITETSEKGLVMSWCPQLDVLAHEAVGCFITHCGWNSVIEATSFGVPMLGMPQFMDHFLDAHFLEKVWGVGIRVKADEKNFVTCDEIKRGLEKIIYGERGNKIKENATKWKELAKEAVGEGGSSDNSTGEIIKWLASS

SEQ ID NO: 124 - protein, 358 aa, Spinacia oleracea
MDRTRPEVESILSTQKPDLVFYDMAHWIPDIASKLGIKSVSYNSVCAISMSHVRPSVLPKAAATSQVALSSSSYVGSVPQWSRNDHNSSVYFGDEITLLERSIISLSSADAIAIRTCREIEGVYCDRVAATFNKPVLVTSHALPDLELQLSPLETRWADWLARFGPRSVIFCCLGSQHVLDAAQLQELALGLEMTGLPFLMAVKPPVGYASLKELLPEGYNDRVSGRGVVHGGWVQQQQILAHPSLGCFVTHCGSSSMWEGLVSETQLVLLPQLPDQTLNAKLMADELKVGVKVERQQNGWVSKQGLCEAINRVMDEDSDISHVVRKNHAKYTSMLISPDFITGYIDNFIKDLQALIS

SEQ ID NO: 125 - DNA, 2085 bp, Pisum sativum
  source 1..2085: mol_type = genomic DNA
atggcaaccttcacacttcacaaagaaacagttcaatcatggttacttctaagtagaactcacatattattccacttcatatgtgtcttgtttctcttttattatcgtatcaaaaatttcattgtttcatatccatggattttaatgacactagctgaaactattctatcagttatgtggtttttcaaccaagcgtaccggtggcggccggtgagtcgttcggttatgaccgagaaactcccggccgaccagaatttaccgggactcgacatatttgtgtgtacgatcgatcctgagaaagaaccaaccgtcgaagttatgaacacggttgtttcggctgttgctatggattaccctagtgataaactttgtgtttatctttctgatgatggtggttctcctgtcactttatatggaattaaagaggcttctcaatttgctaaagcttgggttcctttttgtaaaaaatatggtgttaaatcaaggtgccctaaggtttttttctctcctttgggtgaggatgaacatgttcttaggtcacatgaatttgaagtagaaagagaccagattaaggtgaagtatgagaaaatgcagaaaaatattgagaaattcagttctgatccaaagaatctttgtatggttaatgacagagcttctcggattgagattataaatgaggaatcagaaattccacgtgtgatttatgtgtctcgtgaaagaagaccatcactccctcacaagttcaaaggaggtgccctcaatacattgcttagagtgtcaggtttgataagtaatgggccttatgtacttgcagtggactgtgatatgtattgcaatgatccatcatcagccaaacaagcaatgtgtttctttcttgatccagaaacctctaaatatattgcttttgttcaattccctcaaatgtttcacaatcttagcaaaaaggacatctatgataaccaatccagaactgcttttaagacaatgtggcaagggatggatggattgagaggtccaggtctttctggaagtggtaattacttgaatagaagtgcattattatttggaagtccaaatcaaaaagatgagtatcttcatgatgccaaaaactactttggaggatcaaccacatacatagaatcactcaaggccattcgcggacaacaagttatcaaaaagaatatttccaaagaagaaattttgcaagaagctcaagtagtttcatcttgctcctatgaatcaaacacaaaatggggcacagaaataggattctcatacggaattttactagagagtactatcactggctatcttttacattgtagaggatggaaatcagcatacctttatccaaaaacaccttgtttcttagggtgtgcaccaactgatatcaaagaaggaatgcttcaattagtgaaatggttgtctgaactttgcttgcttgctatctctaaatatagtccttttacttatggattttcaagaatgtcaactattcataacttcacttattgcttcatgtcaatttcatctatatatgctattgggttcatcctttatggtattgtgcctcaagtttgcttcttcaaaggaattcctgtatttccaaaggtcacagatccttggtttgtagtgtttgcaatactatacatagccacacaaattcaacatttaatagaggtaatttctggcgatggctcagtttcaatgtggtgggacgaacaaagaatctggattctaaaatcagttacaagtttatttgcaatgatagaggcagttaagaaatggttaggattgaacaagaaaaaattcaacttgtcaaacaaagcaattgatacagacaaagaaaaaatcaagaaatatgaacaaggtaggtttgattttcaaggtgcggctttgtatatgtctccaatgattgtgttgctcattgtcaacactatttgtttctttggtggtttatggaggcttttcaaaataagagattttgaagatatgtttggccaacttttcttagtaagttacgtcatggctcttagttatccaatttttgaagggattttaactatgaaaagtaagagtggatag

SEQ ID NO: 126 - protein, 694 aa, Pisum sativum
MATFTLHKETVQSWLLLSRTHILFHFICVLFLFYYRIKNFIVSYPWILMTLAETILSVMWFFNQAYRWRPVSRSVMTEKLPADQNLPGLDIFVCTIDPEKEPTVEVMNTVVSAVAMDYPSDKLCVYLSDDGGSPVTLYGIKEASQFAKAWVPFCKKYGVKSRCPKVFFSPLGEDEHVLRSHEFEVERDQIKVKYEKMQKNIEKFSSDPKNLCMVNDRASRIEIINEESEIPRVIYVSRERRPSLPHKFKGGALNTLLRVSGLISNGPYVLAVDCDMYCNDPSSAKQAMCFFLDPETSKYIAFVQFPQMFHNLSKKDIYDNQSRTAFKTMWQGMDGLRGPGLSGSGNYLNRSALLFGSPNQKDEYLHDAKNYFGGSTTYIESLKAIRGQQVIKKNISKEEILQEAQVVSSCSYESNTKWGTEIGFSYGILLESTITGYLLHCRGWKSAYLYPKTPCFLGCAPTDIKEGMLQLVKWLSELCLLAISKYSPFTYGFSRMSTIHNFTYCFMSISSIYAIGFILYGIVPQVCFFKGIPVFPKVTDPWFVVFAILYIATQIQHLIEVISGDGSVSMWWDEQRIWILKSVTSLFAMIEAVKKWLGLNKKKFNLSNKAIDTDKEKIKKYEQGRFDFQGAALYMSPMIVLLIVNTICFFGGLWRLFKIRDFEDMFGQLFLVSYVMALSYPIFEGILTMKSKSG

SEQ ID NO: 127 - DNA, 4509 bp, Pisum sativum
  source 1..4509: mol_type = genomic DNA
caaataaattaggataataatataatttcttattaaacgcgtggacatatatatatatataggaatagacatatattagtaaagaaactaactaattcctataaaaaagaaaagaaaaaacctaattattttattaatctaaacacaacaattatctcaacaacaaaacaaaaatttaaacataattatgaggatttcattttaagatacaaatacttatgcactttgaattcttgaagaagttccactatccactcttacttttcatagttaaaatcccttcaaaaattggataactaagagccatgacgtaacttactaagaaaagttggccaaacatatcttcaaaatctcttattttgaaaagcctccataaaccaccaaagaaacaaatagtgttgacaatgagcaacacaatcattggagacatatacaaagccgcaccttgaaaatcaaacctaccttgttcatatttcttgattttttctttgtctgtatcaattgctttgtttgacaagttgaattttttcttgttcaatcctaaccatttcttaactgcctctatcattgcaaataaacttgtaactgattttagaatccagattctttgttcgtcccaccacattgaaactgagccatcgccagaaattacctctattaaatgttgaatttgtgtggctatgtatagtattgcaaacactacaaaccaaggatctgtgacctgaagtatcaatcattgaaaatatgttaaattattataaaatatattacaaaaatagaattagtgttaaagaaatgtggttgatcctaactcatctatgcaaaataggtttgtaaggtgagaatctctcctacttataaacatgttcaaaatatatattattcgatgtgagactcttaaaatatctatcatgacaaagactgaatatatggaaggtggaaataaatggttggtggttcgatagtagaaacctaatagtaggtggctcaacaaatcttaaatgttgttagacttaactcatccctacgaaactagctagtagagttaggatcgtgccacttataagtacatgttcaaacatgtaacacttttaacaactagtaaaaagaattgagttggagagctttactacctttggaaatacaggaattcctttgaagaagcaaacttgaggcacaataccataaaggatgaacccaatagcatatatagatgaaattgacatgaagcaataagtgaagttatgaatagttgacattcttgaaaatccataagtaaaaggactatatttagagatagcaagcaagcaaagttcagacaaccatttcactaattgaagcattccttctttgatatcagttggtgcacaccctaagaaacaaggtgtttttggataaaggtatgctgatttccatcctctacaatgtaaaagatagccagtgatagtactctctagtaaaattccgtatgagaatcctatttctgtgccccattttgtgtttgattcataggagcaagatgaaactacttgagcttcttgcaaaatttcttctttggaaatattctttttgataacttgttgtccgcgaatggccttgagtgattctatgtatgtggttgatcctccaaagtagtttttggcatcatgaagatactcatctacaatcatgaacagaagaaatcataagaaaaaatctccaataaaaacctttttgtaatacataatgaggcaatacgtacctttttgatttggacttccaaataataatgcacttctattcaagtaattaccacttccagaaagacctggacctctcaatccatccatcccttgccacattgtctaaaataaaatttgcaatatgtcatcattgcataataaacaaaatcggatttcataccgtcaatctttgttgtattcacgctgtcattgttttattaaaagttcgatcttaattggacgatcatgatcaaatgacaaaaacactatataaggactaactgcaaattgcagtcccttgaaaaaaatgaaaaatctaaatgaagaacttcattattattttttatttaccttaaaagcagttctggattggttatcatagatgtcctttttgctaagattgtgaaacatttgagggaattgaacaaaagcaatatatttagaggtttctggatcaagaaagaaacacattgcttgtttggctgatgatggatcattgcaatacatatcacagtccactgcaagtacataaggcccattacttatcaaacctgacactctaagctgcaaacacaaacaaaaatatgttatattttattaactgcatcacaaattaaattttaatatgatatacacaaaatacacaccaatgtattgagggcacctcctttgaacttgtgagggagtgatggtcttctttcacgagacacataaatcacacgtggaatttctgattcctcatttataatctaaaataataaatataaagtaatattaaaaaaatatttttattttattttttactgttaataaataataaattttgattttttaattatttaaaaaataacttcttcaacacactcaaataaacaccaaaatataacaactaaataattgtagaaatatgaaatacactttatttatttatttatttatttgttgttaatttattattgtattcgatctaaaacattttattatatatactatatatatatatagtatatatattatatatatatatataatatatatatatatatatatttatatattattctaccaaattagattgtagtttttgaatttgtggtttaatataattaagtgacaaataactattgcacgacagtgtcaaaataaatgcatcacaattaaaccttataaataatttaaattaattttaaaatagatatgacttatttaaataagtctgatccaacttaaatattttaacttaatttatattaatcataacatatttgtttgtttttctgtgtataaattgagtattatgttctatatttttaacaattttttatttttttattttatcaaataattatactatatatatatataatatatatatatatatatatatattatatatatatatatatatatataatatatatatatatgataatatatatataatttatctaaaaaattaaaatttaattataatataaaaaagagagagtattctcttaaaatataggaatgcaatgttttcaagttgaaattgaatcaaaacttttgtcaaactatttaaatataaatttggtgaattgaaaaatatttgttaaaagattgagtttgaaaacttaaaacctttaattaatatatttgataatatttatcttgttttttctattagtgaaatatatttagactgcttttatacctaattaactagtgaaaaaaacaaataaaccttactaactgtaaatatttacacataaaaacatttatttattttccgttaactagttaactagtaattattccatcaaattgattattgatcataatccaactttagtttttattagtcatacctcaatccgagaagctctgtcattaaccatacaaagattctttggatcagaactgaatttctcaatatttttctgcattttctcatacttcacctgtcaaattgttaaaacatggtatcaatacaaattatgttaactaattttatatatatatcagctatataaaccatataaaataccttaatctggtctctttctacttcaaattcatgtgacctaagaacatgttcatcctcacccaaaggagagaaaaaaaccttagggcaccttgatttaacaccatattttttacaaaaaggaacccaagctttagcaaattgagaagcctctttaattccatataaagtgacaggagaaccaccatcatcagaaagataaacacaaagtttatcactagggtaatccatagcaacagccgaaacaaccgtgttcataacttcgacggttggttctttctcaggatcgatcgtacacacaaatatgtcgagtcccggtaaattctggtcggccgggagtttctcggtcataaccgaacgactcaccggccgccaccggtacgcttggttgaaaaaccacataactgatagaatagtttcagctagtgtcattaaaatccatggatatgaaacaatgaaatttttgatacgataataaaagagaaacaagacacatatgaagtggaataatatgtgagttctacttagaagtaaccatgattgaactgtttctttgtgaagtgtgaaggttgccataacaagtcaatacttgaatatttttcacaacaaaattaagttgcgtatggtttataataggtgaagtttcatttatatataatgtatctatcacttgttagttgttgaagcaaaataaataaataaattctatagtcattagaaatgaaaatcaaatactaattttaaagtaactctcgtggaactcgc

SEQ ID NO: 128 - DNA, 23 bp, synthetic construct
  source 1..23: mol_type = other DNA
ccggccgccaccggtacgcttgg

SEQ ID NO: 129 - DNA, 23 bp, synthetic construct
  source 1..23: mol_type = other DNA
gagtcccggtaaattctggtcgg

SEQ ID NO: 130 - DNA, 23 bp, synthetic construct
  source 1..23: mol_type = other DNA
taccgtcgaagttatgaacacgg